(19) United States
(12) Patent Application Publication
(10) Pub. No.: US 2003/0003502 A1
(43) Pub. Date: Jan. 2, 2003

(54) Three-dimensional model of a complex between a Fc epsilon receptor alpha chain and a Fc region of an IgE antibody and uses thereof

(76) Inventors: Theodore S. Jardetzky, Chicago, IL (US); Scott Clayton Garman, Rockville, MD (US); Beth A. Wurzburg, Evanston, IL (US); Jean-Pierre Kinet, Lexington, MA (US)

Correspondence Address: HESKA CORPORATION, INTELLECTUAL PROPERTY DEPT., 1613 PROSPECT PARKWAY, FORT COLLINS, CO, 80525, US

(21) Appl. No.: 09/809,715

(22) Filed: Mar. 14, 2001

Related U.S. Application Data
(60) Provisional application No. 60/189,853, filed on Mar. 15, 2000.

(51) Int. Cl. G01N 33/53; G06G 7/48; G06G 7/58; G06F 19/00

(52) U.S. Cl. 435/7.1; 702/19; 703/11

(57) ABSTRACT

The present invention includes three-dimensional models of complexes between antibody receptor proteins, such as FcεRIα proteins, and antibodies, such as Fc-Cε3/Cε4 regions of IgE antibodies, as well as methods to produce such models. The present invention also includes muteins having increased stability and/or antibody binding activity, as well as methods to produce such muteins, preferably using information derived from three-dimensional models of the present invention. Also included are nucleic acid sequences encoding muteins of the present invention and use of those sequences to produce such muteins. Also included is the use of the model to identify compounds that inhibit the binding of an antibody receptor protein to an antibody. The present invention also includes uses of such muteins and inhibitory compounds, for example, in methods to diagnose and protect animals from allergy and other abnormal immune responses.

### CROSS-REFERENCE TO RELATED APPLICATION

This Application claims priority to provisional application U.S. Serial No. 60/189,853, filed Mar. 15, 2000, “THREE-DIMENSIONAL MODEL OF A COMPLEX BETWEEN A Fe EPSILON RECEPTOR ALPHA CHAIN AND A Fe REGION OF AN IgE ANTIBODY AND USES THEREOF.”

This invention was made at least in part with government support under NIH Grant No. RO1 A138972, awarded by the National Institutes of Health to Northwestern University. The government has certain rights to this invention.

### FIELD OF THE INVENTION

The present invention relates to a crystal and a three-dimensional (3-D) model of a complex between a Fe epsilon receptor alpha chain (FcεRIα, or FceRIa) protein and a constant region of an IgE antibody that includes the Cε3 and Cε4 domains (Fc-Cε3/Cε4, or Fc-Cε3/Cε4, region). The present invention also relates to the use of that model to produce muteins and inhibitors useful in the diagnosis and treatment of allergy and the regulation of other immune responses in an animal.

### BACKGROUND OF THE INVENTION

Antibody Fc-receptors (FcRs) play an important role in the immune response by coupling the specificity of secreted antibodies to a variety of cells of the immune system. A number of cell types, including macrophages, mast cells, eosinophils, and basophils, express membrane-bound FcRs at their surfaces. The binding of antibodies to FcRs provides antigen-specificity to these cells, which upon activation release further cell-specific mediators of the immune response, such as interleukins, initiators of inflammation, leukotrienes, prostaglandins, histamines, or cytotoxic proteins. The adoptive specificity of the FcRs allows a combinatorial approach to pathogen elimination, by coupling the diversity of antibody antigen-recognition sites to the variety of cell-types expressing these receptors.

FcR-initiated mechanisms are important in normal immunity to infectious disease as well as in allergies, antibody-mediated tumor recognition, autoimmune diseases, and other diseases in which immune responses are abnormal (i.e., not regulated). Recent experiments with transgenic mice have demonstrated that the FcRs control key steps in the immune response, including antibody-directed cellular cytotoxicity and inflammatory cascades associated with the formation of immune complexes; see, for example, Ravetch et al., 1998, Annu Rev Immunolo 16, 421-432. Receptors that bind IgG (FcgRI, FcgRII, and FcgRIII, known collectively as FcgRs) mediate a variety of inflammatory reactions, regulate B-cell activation, and also trigger hypersensitivity reactions. The high affinity Fc epsilon receptor (also known as the IgE receptor or FceRI) is associated with the activation of mast cells and the triggering of allergic reactions and anaphylactic shock. Knockout mice for the FceRI alpha chain (FcεRIα) are unable to mount IgE-mediated anaphylaxis (see for example, Dombrowicz et al., 1993, Cell 75, 969-976), although FcgRs are still able to activate mast cells (see, for example, Dombrowicz et al., 1997, J. Clin. Invest. 99, 915-925; Oettgen et al., 1994, Nature 370, 367-370). FceRI has also been shown to trigger anti-parasitic reactions from platelets and eosinophils as well as deliver antigen into the MHC class II presentation pathway for the activation of T cells; see, for example, Gounni et al., 1994, Nature 367, 183-186; Joseph et al., 1997, Eur. J. Immunol. 27, 2212-2218; Maurer et al., 1998, J. Immunol. 161, 2731-2739. The beta subunit of FceRI has been associated with asthma in genetic studies; see, for example, Hill et al., 1996, Hum. Mol. Genet. 5, 959-962; Hill et al., 1995, Bmj 311, 776-779; Kim et al., 1998, Curr. Opin. Pulm. Med. 4, 46-48; Mao et al., 1998, Clin. Genet. 53, 54-56; Shirakawa et al., 1994, Nat. Genet. 7, 125-129. A significant fraction of the population (˜20%) may be affected by allergies, and this century has seen a substantial increase in asthma. Since IgE binding to FceRI is a requisite event in the reaction to different allergens, therapeutic strategies aimed at inhibiting FceRI could provide a useful treatment for these diseases. For example, monoclonal antibodies that target IgE and block receptor binding have shown therapeutic potential; see, for example, Heusser et al., 1997, Curr. Opin. Immunol. 9, 805-813.

FceRI is found as a tetrameric (abg₂) or trimeric (ag₂) membrane bound receptor on the surface of mast cells, basophils, eosinophils, langerhans cells and platelets. The alpha chain, also referred to as FcεRIα, of FceRI binds IgE molecules with high affinity (K_{D} of about 10⁻⁹ to 10⁻¹⁰ moles/liter (M)), and can be secreted as a 172-amino acid soluble, IgE-binding fragment by the introduction of a stop codon before the single C-terminal transmembrane anchor; see, for example, Blank et al., 1991, E. J. Biol. Chem. 266, 2639-2646, which describes the secretion of a soluble IgE-binding fragment of 172 amino acids. The extracellular domains of the human FcεRIα protein belong to the immunoglobulin (Ig) superfamily and contain seven N-linked glycosylation sites. Glycosylation of FcεRIα affects the secretion and stability of the receptor, but is not required for IgE-binding; see, for example, LaCroix et al., 1993, Mol. Immunol. 30, 321-330; Letourneur et al., 1995, J. Biol. Chem. 270, 8249-8256; Robertson, 1993, J. Biol. Chem. 268, 12736-12743; Scarselli et al., 1993, FEBS Lett 329, 223-226. The beta and gamma chains of FceRI are signal transduction modules.

Prior investigators have disclosed the nucleic acid sequence for human FcεRIα; see, for example, U.S. Pat. No. 4,962,035, by Leder, issued Oct. 9, 1990; U.S. Pat. No. 5,639,660, by Kinet et al., issued Jun. 17, 1997; Kochan et al., 1988, Nucleic Acids Res. 16, 3584; Shimizu et al., 1988, Proc. Natl. Acad. Sci. USA 85, 1907-1911; and Pang et al., 1993, J. Immunol. 151, 6166-6174. Nucleic acid sequences have also been reported for the human FceRI beta and gamma chains; see, respectively, Kuster et al., 1992, J. Biol. Chem. 267, 12782-12787; Kuster et al., 1990, J. Biol. Chem. 265, 6448-6452. Nucleic acid sequences have also been reported for nucleic acid molecules encoding canine FcεRIα, murine FcεRIα, rat FcεRIα, feline FcεRIα and equine FcεRIα proteins; see, respectively, GenBank™ accession number D16413; Swiss-Prot accession number P20489 (represents encoded protein sequence); GenBank accession number J03606; PCT Publication No. WO 98/27208, by Frank et al., published Jun. 25, 1998, referred to herein as WO 98/27208; and PCT Publication No. WO 99/38974, by Weber et al., published Aug. 5, 1999, referred to herein as WO 99/38974. In addition, methods to detect IgE antibodies using a FcεRIα protein have been reported in PCT Publication No. WO 98/23964, by Frank et al., published Jun. 4, 1998, referred to herein as WO 98/23964; WO 98/27208, ibid.; PCT Publication No. WO 98/45707, by Frank et al., published Oct. 15, 1998, referred to herein as WO 98/45707; and WO 99/38974, ibid. WO 98/23964, WO 98/27208, WO 98/45707 and WO 99/38974 are each incorporated by reference herein in its entirety.

There have been several reports of the use of mutagenesis and swapping techniques to attempt to identify amino acids of either FcεRIα or IgE involved in the binding of (i.e., interaction between) those respective proteins, reports attempting to model FcεRIα proteins based on homology to other Ig-superfamily members, and reports that identify compounds that apparently inhibit such binding; see, for example, Cook et al., 1997, Biochemistry 36, 15579-15588; Hulett et al., 1994, J. Biol. Chem. 269, 15287-15293; Hulett et al., 1995, J. Biol. Chem 270, 21188-21194; Mallamaci et al., 1993, J. Biol. Chem. 268, 22076-22083; Robertson, 1993, ibid.; Scarselli et al., 1993, ibid. McDonnell et al., 1997, Biochem. Soc. Trans. 25, 387-392; McDonnell et al., 1996, Nat. Struc. Biol. 3, 419-426; PCT Publication No. WO 97/40033, by Cheng et al., published Oct. 30, 1997; U.S. Pat. No. 5,180,805, by Gould et al, issued Jan. 19, 1993; U.S. Pat. No. 5,693,758, by Gould et al., issued Dec. 2, 1997; PCT Publication No. WO 96/01643, by Gould et al., published Jan. 25, 1996; PCT Publication No. WO 95/14779, by Gould et al., published Jun. 1, 1995. None of these references, however, describe isolated crystals of FcεRIα proteins or 3-D models derived from crystals.

Despite what is known about FcRs and their interaction with antibodies, there remains a need for FcRs and antibodies with improved characteristics, such as enhanced affinity for their ligands, altered substrate specificity, increased stability, and increased solubility for use in diagnosis, treatment and prevention of allergy and other abnormal immune responses. Also needed for safe and efficacious compounds to prevent or treat allergy and to regulate other immune responses in an animal.

### SUMMARY OF THE INVENTION

The present invention includes isolated crystals of a complex between the extracellular domains of antibody receptor proteins (FcRs) and constant regions (Fc regions) of antibodies, three-dimensional (3-D) models of such crystals and modifications of such models. The present invention also includes compounds that inhibit the ability of FcRs to bind to antibodies as well as FcR muteins and other modified FcRs as well as antibody muteins and other modified antibodies. Also included in the present invention are methods to produce and use such crystals, models, inhibitory compounds, muteins, and other modified proteins. As such, the present invention includes FcRs and antibodies with improved functions such as increased stability, increased affinity for an Fc domain of an antibody, altered substrate specificity, and increased solubility, including but not limited to reduced aggregation. Such proteins, also referred to as muteins, are useful to detect allergy and other immune response abnormalities as well as to protect an animal from such abnormalities. The present invention also provides safe and efficacious inhibitory compounds to protect (e.g., prevent, treat, reduce the consequences of) an animal from allergy and to regulate other immune responses in an animal.

The present invention includes a 3-D model of a complex between an extracellular domain of a human high affinity Fc epsilon receptor alpha chain (FcεRIα) protein and a human IgE Fc region comprising Cε3 and Cε4 domains, wherein the model substantially represents the atomic coordinates specified in Table 1. The present invention also includes a 3-D model comprising a modification of a model substantially representing the atomic coordinates specified in Table 1. Also included in the present invention are methods to produce such models.

The present invention also includes an isolated crystal of a complex between an extracellular domain of a human high affinity Fc epsilon receptor alpha chain protein and a human IgE Fc region comprising Cε3 and Cε4 domains.

The present invention includes a method to identify a compound that inhibits the binding between an IgE antibody and a FcεRIα protein. The method includes the step of using a 3-D model of the present invention, and particularly one substantially represents the atomic coordinates specified in Table 1. Also included in the present invention are inhibitory compounds identified using such a method. Also included are therapeutic compositions that include such inhibitory compounds and methods to use such therapeutic compositions to protect an animal from allergy or to regulate other immune responses (e.g., protect an animal from other abnormal immune responses).

The present invention also includes a mutein that binds to a Fc domain of an antibody or to a Fc binding domain of a FcR. Such a mutein has an improved function compared to a protein that includes SEQ ID NO:2 or SEQ ID NO:6, respectively. Examples of such an improved function include increased stability, increased affinity for an Fc domain of an antibody, altered substrate specificity, decreased aggregation, and increased solubility. Such a mutein is produced by a method that includes the following steps: (a) analyzing a 3-D model substantially representing the atomic coordinates specified in Table 1 to identify at least one amino acid of the protein represented by the model which if replaced by a specified amino acid would effect an improved function of the protein; and (b) replacing the identified amino acid(s) to produce the mutein having such an improved function. The present invention also includes a mutein having an improved function compared to an unmodified FcεRIα protein or IgE Fc region.

Also included are muteins that are chemically modified FcεRIα proteins or antibodies. Also included are nucleic acid molecules that encode muteins of the present invention, recombinant molecules and recombinant cells including such nucleic acid molecules and methods to produce such muteins. Also included are diagnostic reagents and diagnostic kits including such muteins, therapeutic compositions including such muteins, and methods to detect or protect an animal from allergy or other abnormal immune responses.

The present invention also includes a method to improve a function of a FcεRIα protein or IgE Fc region which includes the steps of: (a) analyzing a 3-D model substantially representing the atomic coordinates specified in Table 1 to identify at least one amino acid of the protein which if replaced by a specified amino acid improves at least one of the functions of the protein; and (b) replacing the identified amino acid(s) to produce a mutein having at least one of the improved functions.

### BRIEF DESCRIPTION OF THE DRAWINGS

FIG. 1 shows an electron density map and ribbon diagrams depicting the overall structure of the IgE-Fc:FcεRIα complex. FIG. 1 a shows a stereo diagram from a σₐ-weighted 2Fₒ-F_{c} simulated annealing omit electron density map at 3.5 angstroms. The complex is contoured at 1.25σ. FcεRIα residues 129-136 of FcεRIα and IgE-Fc loop residues 334-336 and 362-364 are shown. FIG. 1b is a side view of the IgE-Fc:FcεRIα complex depicting the two Fc chains (yellow and red ribbon, upper left of figure) and the FcεRIα chain (blue ribbon, lower right of figure). Binding sites 1 and 2 are indicated. The cell membrane would lie below the receptor. FIG. 1c is a top view of the IgE-Fc:FcεRIα complex shown in FIG. 1b.

FIG. 2 shows a surface representation of the IgE-Fc:FcεRIα complex. FIG. 2a is a side view of the IgE-Fc:FcεRIα complex highlighting how the convex surface of the receptor interacts asymmetrically with the two IgE-Fc Cε3 domains. The two Fc chains are in yellow and red while the FcεRIα chain is in blue. Carbohydrate surfaces are white, detergent surface is black. FIG. 2b is a top view of the IgE-Fc:FcεRIα complex surface representation shown in FIG. 2a. FIG. 2c is a superposition of the two IgE-Fc Cε3 domains. The twofold symmetry of the IgE-Fc domains is broken in the Cε2-Cε3 linker region (residues 328-336) by interactions with the receptor. Superposition of the Cε3 domains leads to a small displacement in the Cε4 domain, because of a 3° difference in Cε3 and Cε4 pseudo-dyad axes. FIG. 2d is a surface representation of both IgE-Fc and FcεRIα in which the IgE-Fc:FcεRIα complex has been separated to expose the surfaces involved in binding. The IgE (upper left) is oriented to give an end-on view of the Cε3 domains. Binding residues that bind FcεRIα are shown in yellow (Site 1) and red (Site 2). A top and side view of the FcεRIα is shown on the right-hand side of FIG. 2d. Residue Y131 of site 1 and the binding pocket for P426 of the IgE-Fc are labeled. Carbohydrate is shown in grey.

FIG. 3 details the interactions in the IgE-Fc:FcεRIα complex at Site 1 and Site 2. FIG. 3a is a plot showing the buried surface area of residues in the IgE-Fcε3 domains. The top half of the graph shows residues buried in the Site 1 interaction (yellow bars), while the bottom half of the graph shows residues buried in the Site 2 interaction (red bars). The IgE loops are identified above the plot. 50 Å of buried surface area of N394 is due to attached carbohydrate. FIG. 3b is a stick model diagram of residue interactions at Site 1. The IgE-Fc and FcIa chains are tan and blue, respectively. Binding loops are labeled at their termini, side chains of residues buried in the complex are shown and Y131 is labeled. FIG. 3c is a stick model diagram of the residue interactions at Site 2. The IgE-Fc and FcεRIα chains are red and blue, respectively. Side chains of residues buried in the complex are shown. FIG. 3d is a space filling model showing binding of CHAPS detergent molecule in the IgE-Fc:FcεRIα complex. Atoms less than 4A apart have dotted lines between them and the residues are labeled. No density appears for the flexible top-end of the detergent and those atoms are not labeled.

FIG. 4 illustrates the conservation of amino-acid residues and contacts at the IgE-Fc:FcεRIα interfaces in IgG receptors and antibodies. Contacting residues are defines as interatomic distances <4 Å. FIG. 4a shows the Site 1 interacting residues and their conservation in related human receptors and antibodies. Absolutely conserved residues are highlighted in bold and partially conserved residues are lightly highlighted (yellow for IgE, blue for FcεRIα). Dark lines are drawn for residues making the largest number of contacts across the interface, lighter lines for intermediate number of contacts, and dashed lines for the fewest contacts. FIG. 4b shows the Site 2 interacting residues and their conservation in human related Fc receptors and antibodies. Receptor residues are highlighted in blue, antibody residues in red. Three residues in IgG2 (PVA) that disrupt binding to FcγRI are boxed in black. FIG. 4c is a closeup of the Site 2 trp/proline interaction (FcR surface with IgE-ribbon interaction). Also shown are residues implicated in the IgG specificity between different receptor subtypes (corresponding to residues 332-334 in IgE) that interact with the FG loop. FIG. 4d is shows how FcRY131 in Site 1 interacts with a shallow pocket on the Cε3 domain that could be a source of specificity for IgG interactions (Y changes to H or R in FcγRII and FcγRIII).

FIG. 5 depicts a kinetic scheme for the binding of IgE to its receptor. The interaction of each Cε3 domain with distinct surfaces of the FcεRIα structure suggests a kinetic scheme in which transient release of one of the Cε3 domains may occur within the complex. This could lead to two distinct pathways for the association and dissociation of the complex, consistent with the experimental observation of two distinct off-rates. Transient opening of the complex may allow inhibitors to enhance the dissociation of receptor-bound IgE by preventing the re-binding of an exposed Cε3 domain within the complex.

FIG. 6 is a ribbon-model showing the superposition of the Fc portion of an intact IgG antibody (1IGY)27 and IgG Fc receptor FcγRII22 onto the IgE-Fc:FcεRIα complex. The IgE complex is shown in beige and the IgG homologues in blue. Only a minor adjustment of the other IgG domain is required to fit the IgE complex.

FIG. 7 shows a hypothetical model for an intact IgE:Fc receptor complex. The Fc chains are in red and yellow, the FcεRIα chain is in blue. Antibody Fab regions are shown in beige.

### DETAILED DESCRIPTION OF THE INVENTION

The present invention includes isolated crystals of complexes between the extracellular domains of FcRs and Fc regions of antibodies, 3-D models of such crystals and modifications of such models. The present invention also includes compounds that inhibit the ability of FcRs to bind to antibodies as well as muteins and other modified FcRs and antibodies. Also included in the present invention are methods to produce and use such crystals, models, inhibitory compounds, muteins, and other modified proteins.

The present invention includes an isolated crystal of a complex between an extracellular domain of a high affinity Fc epsilon receptor alpha chain (FcεRIα) and a Fc region comprising the Cε3 and Cε4 domains of an IgE antibody (Fc-Cε3/Cε4), a 3-D model of such a crystal and a modification of such a model. As used herein, the term “a” entity or “an” entity refers to one or more of that entity; for example, a crystal or a model refers to one or more crystals or models, respectively. As such, the terms “a” (or “an”), “one or more” and “at least one” can be used interchangeably herein. It is also to be noted that the terms “comprising”, “including”, and “having” can be used interchangeably. Furthermore, a compound “selected from the group consisting of” refers to one or more of the compounds in the list that follows, including mixtures, or combinations, of two or more of the compounds.

As used herein, an extracellular domain of a FcεRIα protein is the portion of the FceRI alpha chain that is exposed to the environment outside the cell and that binds to the Fc domain of an IgE antibody. Such an extracellular domain can be (a) a complete extracellular domain which is a domain that extends from the first amino acid of a mature FceRI alpha chain through the last amino acid prior to the start of the transmembrane region or a domain that is functionally equivalent, in that such a domain includes a D1 and D2 domain, displays a similar affinity for the IgE antibody to which such an FcεRIα protein naturally binds, and produces crystals having sufficient quality to enable structure determination, or (b) a fragment of any of the extracellular domains of (a), wherein the fragment retains its ability to bind to the Fc domain of an antibody. As used herein, the terms binding to an antibody and binding to the Fc domain (i.e., constant region) of an antibody can be used interchangeably since it is recognized that a FcR binds to the Fc domain of an antibody. A FcR (i.e., a protein that can bind to an antibody), such as a FcεRIα protein, can be a full-length FcR (e.g., a full-length FceRI alpha chain), or any fragment thereof, wherein the fragment binds to an antibody. Similarly an antibody, or an Fc region thereof, can be a full-length antibody, or full-length Fc region thereof, or any fragment thereof that binds to a FcR. In one embodiment an Fc region comprises Cε3 and Cε4 domains. Preferably a FcR binds to an antibody with an affinity (K_{A}) of at least about 10⁸ liters/mole (M⁻¹), more preferably of at least about 10⁹ M⁻¹, and even more preferably of at least about 10¹⁰ M⁻¹.

The present invention is surprising in several aspects. For example, this is the first report of an isolated crystal of a complex between an extracellular domain of a FcεRIα protein and a Fc-Cε3/Cε4 region of an IgE antibody, and in particular of an isolated crystal of sufficient quality that a crystal structure, i.e., a 3-D model, could be derived therefrom. Generation of such a crystal was very difficult and non-obvious and has been attempted by others without success. The inventors tried many approaches before discovering a preferred FcεRIα protein and a preferred Fc-Cε3/Cε4 region from which to make a useful crystal. Part of the reason for the difficulty is that the FcεRIα protein is highly glycosylated. Although crystals could be produced using a FcεRIα protein that consists of amino acids 1 through 176 of the mature human FcεRIα protein, a protein that is denoted herein as PhFcεRIα₁₋₁₇₆, or the hFcεRIα₁₋₁₇₆ protein, and has an amino acid sequence denoted herein as SEQ ID NO:2, much better crystals could be generated using a FcεRIα protein that consists of amino acids 1 through 176 of the mature human FcεRIα protein that had been mutated to replace four N-linked glycosylation sites with other amino acids at positions 74, 135, 142 and 143 of SEQ ID NO:2 to produce a protein having SEQ ID NO:4, the protein being denoted herein as PhFcεRIα₁₋₁₇₆ₘᵤₜ, or the hFcεRIα₁₋₁₇₆ₘᵤₜ protein. An example of a nucleic acid molecule encoding PhFcεRIα₁₋₁₇₆ is referred to herein as nhFcεRIα₁₋₅₂₈, the nucleic acid sequence of which is denoted herein as SEQ ID NO:1. An example of a nucleic acid molecule encoding PhFcεRIα₁₋₁₇₆ₘᵤₜ is referred to herein as nhFcεRIα₁₋₅₂₈ₘᵤₜ, the nucleic acid sequence of which is denoted herein as SEQ ID NO:3. Identification of an appropriate Fc-Cε3/Cε4 region to crystallize was also difficult. The first such region to be used successfully is referred to herein as PhFc-Cε3/Cε4₁₋₂₂₂ which is composed of the four amino acids alanine, aspartic acid, proline and cysteine at the amino terminus followed by amino acids 330 through 547 of the human IgE Fc constant region, using the numbering system of Dorrington et al, 1978, Immunol Rev 41, 3-25. PhFc-Cε3/Cε4₁₋₂₂₂ is represented herein by SEQ ID NO:6. An example of a nucleic acid molecule encoding PhFc-Cε3/Cε4₁₋₂₂₂ is referred to herein as nhFc-Cε3/Cε4₁₋₆₆₆, the nucleic acid sequence of which is referred to herein as SEQ ID NO:5. It was also discovered that better crystals are generated when PhFcεRIα₁₋₁₇₆ and PhFc-Cε3/Cε4₁₋₂₂₂ are produced in insect cells, using a method such as that described in the Examples.

Determination of the crystal structure of the complex between PhFcεRIα₁₋₁₇₆ₘᵤₜ and PhFc-Cε3/Cε4₁₋₂₂₂, each produced in Trichoplusia ni (Hi-5) cells, resulted in a 3-D model that substantially represents the atomic coordinates specified in Table 1. Amino acids are represented herein by their standard three or one letter codes; see, for example, Sambrook et al., Molecular Cloning: A Laboratory Manual, Cold Spring Harbor Labs Press, 1989, which is incorporated herein by reference in its entirety. Prior to obtaining a crystal of sufficient quality to solve its crystal structure using insect-cell produced PhFcεRIα₁₋₁₇₆ₘᵤₜ and PhFc-Cε3/Cε4₁₋₂₂₂, a number of other proteins were tried without success, as described in the Examples. , including a FcεRIα protein spanning from amino acid 1 through 171 of SEQ ID NO:2 produced in Pichia pastoris, and FcεRIα proteins spanning from amino acid 1 through 172 of SEQ ID NO:2 produced in Chinese hamster ovary cells, Trichoplusia ni cells, and Spodoptera frugiperda cells without success. Without being bound by theory, it is believed that PhFcεRIα₁₋₁₇₆ₘᵤₜ was a better candidate because it apparently represents a complete extracellular domain and it lacked carbohydrates that interfered with complex formation for structural analysis.

The 3-D model of the complex between PhFcεRIα₁₋₁₇₆ₘᵤₜ and PhFc-Cε3/Cε4₁₋₂₂₂ is also very surprising in view not only of the knowledge of the structure of proteins containing immunoglobulin domains, herein also referred to as Ig domains, but also in view of the crystal structures of FcεRIα alone, which is disclosed in U.S. patent application Ser. No. 09/434,193, filed Nov. 4, 1999, by Jardetzky et al., and in PCT Publication No. WO 00/26246, published May 11, 2000, by Jardetzky et al., and of Fc-Cε3/Cε4 alone, which is disclosed in U.S. patent application Ser. No. 60/189,403, filed Mar. 15, 2000, by Jardetzky et al. WO 00/26246, ibid., 09/434,193, ibid., and 60/189,403, ibid. are incorporated by reference herein in their entireties. Not only is the structure of FcεRIα in the complex fairly similar to the unique structure of FcεRIα alone, but, even more surprisingly, the structure of Fc-Cε3/Cε4 in the complex is very different from that of Fc-Cε3/Cε4 alone. For example, as disclosed in 60/189,403, ibid., the Fc region of IgE alone exists in a closed conformation whereas receptor-bound IgE Fc exists in an open conformation. The model also predicts that a FcεRIα protein and an IgE Fc region bind at a stoichiometry of 1:1 which is surprising since each Fc region has two Cε3 domains. Comparison of these structural similarities and differences are described in greater detail in the Examples. Analysis of the model which substantially represents the atomic coordinates specified in Table 1 indicates the necessity of such a model for proper interpretation and refinement of mutagenesis and region swapping studies that have been reported. Such a model permits differentiation, even more so than models of FcεRIα alone as disclosed in Ser. No. 09/434,193, ibid., WO 00/26246, ibid., and Garman et al., 1999, Cell 95, 951-961, between amino acids directly or indirectly influencing binding of IgE to FcεRIα and demonstrates where amino acids and amino acid segments identified in mutagenesis and swapping studies are positioned on the protein. By using a model of the present invention one can identify the interactions of FcεRIα and IgE, thereby identifying amino acids to target for mutein production or regions to target for the development of compounds to inhibit binding of IgE to its receptor. Such a model can be used alone or in conjunction with a model of FcεRIα alone (Ser. No. 09/434,193, ibid. or WO 00/26246, ibid.) or Fc-Cε3/Cε4 alone (60/189,403, ibid.).

One embodiment of the present invention is an isolated crystal of a complex between an extracellular domain of a FcεRIα protein and a Fc-Cε3/Cε4 region of an IgE antibody. As used herein, an isolated crystal is a crystal of a protein that has been produced in a laboratory; that is, an isolated crystal is produced by an individual and is not an object found in situ in nature. It is appreciated by those skilled in the art that there are a variety of techniques to produce crystals including, but not limited to, vapor diffusion using a hanging or sitting drop methodology, vapor diffusion under oil, and batch methods; see, for example, Ducruix et al., eds., 1991, Crystallization of nucleic acids and proteins; A practical approach, Oxford University Press, and Wyckoff et al., eds., 1985, Methods in Enzymology 11, 49-185; each reference is incorporated by reference herein in its entirety. It is also to be appreciated that crystallization conditions can be adjusted depending on a protein's inherent characteristics as well as on a protein's concentration in a solution and that a variety of precipitants can be added to a protein solution in order to effect crystallization; such precipitants are known to those skilled in the art. In a preferred embodiment, a crystal of a complex between an FcεRIα protein and a Fc-Cε3/Cε4 region is produced in a solution by adding a precipitant such as polyethylene glycol (PEG) or PEG monomethylether. In one embodiment, a crystal of the present invention is produced in the presence of 3-[3-(cholamidopropyl) dimethylammonio]-1-propane-sulfonate (CHAPS), or a similar detergent. It is also to be noted that a FcεRIα protein and Fc-Cε3/CCε4 region used to produce a crystal can be produced by a variety of methods, including purification of a native protein, chemical synthesis of a protein, or recombinant production of a protein. Although a number of cell types can be used to recombinantly produce such a protein, insect cells, such as, but not limited to Trichoplusia ni and Spodoptera frugiperda, are preferred, with Trichoplusia ni cells being more preferred. Additional methods to produce proteins are disclosed below.

Isolated crystals of the present invention can include heavy atom derivatives, such as, but not limited to, gold, platinum, mercury, selenium, copper, and lead. Such heavy atoms can be introduced randomly or introduced in a manner based on knowledge of 3-D models of the present invention. Additional crystals of the present invention are not derivatized. In one embodiment, an isolated crystal of the present invention is a co-crystal of a FcεRIα protein bound to a Fc domain of an IgE antibody in the presence of a compound that inhibits the binding of a FcεRIα protein to a Fc domain of an IgE antibody. Additional crystals of the present invention include crystals produced from proteins that are muteins of the present invention or other proteins that are represented by a 3-D model of the present invention.

An isolated crystal of the present invention can be the crystal of a complex between any suitable extracellular domain of a FcεRIα protein and a Fc region that binds to FcεRIα, such as a Fe comprising Cε3 domains or a Fe comprising Cε3 and Cε4 domains. Suitable FcεRIα proteins include mammalian FcεRIα proteins, with human, canine, feline, equine, rat and murine FcεRIα proteins being preferred, and human FcεRIα proteins being even more preferred. Suitable Fc-Cε3/Cε4 regions include mammalian Fc-Cε3/Cε4 regions, proteins, with human, canine, feline, equine, rat and murine Fc-Cε3/Cε4 regions being preferred, and human Fc-Cε3/Cε4 regions being even more preferred. A preferred crystal of the present invention diffracts X-rays to a resolution of about 4.5 angstroms or higher (i.e., lower number meaning higher resolution), with resolutions of about 4.0 angstroms or higher, about 3.5 angstroms or higher, about 3.25 angstroms or higher, about 3 angstroms or higher, about 2.5 angstroms or higher, about 2 angstroms or higher, about 1.5 angstroms or higher, and about 1 angstrom or higher being increasingly more preferred. It is appreciated, however, that additional crystals of lower resolutions can have utility in discerning overall topology of the structures, e.g., location of a binding site or where a molecule binds to a receptor or to an antibody. A particularly preferred isolated crystal of the present invention has the amino acid sequence SEQ ID NO:2, amino acid sequence SEQ ID NO:4, or a sequence essentially equivalent that represents an extracellular domain of another mammalian FcεRIα protein in complex with a Fc-Cε3/Cε4 region having amino acid sequence SEQ ID NO:6, or a sequence essentially equivalent that represents another mammalian Fe-Cε3/Cε4 region. Preferred are crystals that belong to spacegroup P4₁2₁2 or spacegroup R32. Particularly preferred crystals include: a crystal belonging to spacegroup P4₁2₁2 that has cell dimensions of 126 angstroms×126 angstroms×129 angstroms and that diffracts X-rays to a resolution of about 4.5 angstroms; and a crystal belonging to spacegroup R32 that has cell dimensions of 192.8 angstroms×192.8 angstroms×302 angstroms and that diffracts X-rays to a resolution of about 3.25 angstroms.

The present invention includes a 3-D model of a complex between an extracellular domain of a FcεRIα protein and a Fc-Cε3/Cε4 region that substantially represents the atomic coordinates specified in Table 1. The present invention also includes 3-D models that comprise modifications of the model substantially represented by the atomic coordinates specified in Table 1. Each such modification represents a complex between a Fc receptor protein that binds to a Fc domain of an antibody and an antibody Fc region that binds to a Fc receptor protein. A 3-D model of a complex between an extracellular domain of a FcεRIα protein and a Fc-Cε3/Cε4 region is a representation, or image, that predicts the actual structure of the corresponding complex. As such, a 3-D model is a tool that can be used to probe the relationship between the complex's structure and function at the atomic level and to design muteins (i.e., genetically and/or chemically altered FcRs or antibodies) having an improved function, such as, but not limited to: increased (i.e., enhanced) stability; increased antibody or FcR, respectively, binding activity, for example, by, increasing the affinity for an antibody or FcR, respectively, by, for example, increasing the association rate and/or decreasing the dissociation rate between a FcR and an antibody or by altering substrate specificity (e.g., enhancing the ability of a FcR of a certain species and class to bind to antibody from another species and/or another antibody class); and/or increased solubility (e.g., reduced aggregation). It is well known to those skilled in the art, however, that a 3-D model of a protein or a complex derived by analysis of protein or complex crystals is not identical to the inherent structure of the protein or complex. See, for example, Branden et al., Introduction to Protein Structure, Garland Publishing Inc., New York and London, 1991, especially on page 277, which states “not surprisingly the model never corresponds precisely to the actual crystal.” Furthermore, the model can be subjected to further refinements to more closely correspond to the actual structure of a complex between a FcR and antibody. Such a refined model, which is an example of a modification of the present invention, is a better predictor of the actual structure and mechanism of action of the complex that the model represents. A refinement of a 3-D model of the present invention refers to an improved model of a complex between an extracellular domain of a FcεRIα protein and a Fc-Cε3/Cε4 region that can be obtained in a variety of ways known to those skilled in the art. Refinements can include models determined to more preferred degrees of resolution, preferably to about 4.5 angstroms, more preferably to about 4 angstroms, more preferably to about 3.5 angstroms, more preferably to about 3.25 angstroms, more preferably to about 3 angstroms, more preferably to about 2.5 angstroms, more preferably to about 2 angstroms, more preferably to about 1.5 angstroms, and even more preferably to about 1 angstrom. Preferred refinements are obtained using the 3-D model as a basis for such improvements.

One embodiment of the present invention is a 3-D model of a complex between an extracellular domain of a FcεRIα protein and a Fc-Cε3/Cε4 region that substantially represents the atomic coordinates specified (i.e., listed) in Table 1.

TABLE 1
Atomic coordinates of com14i_deposit.pdb

| ATOM | ATOM | | | | | | | | |
|---|---|---|---|---|---|---|---|---|---|
| # | TYPE | RES | CHN | # | X | Y | Z | OCC | B |
| 1 | CB | VAL | A | 1 | −3.308 | 77.955 | 157.480 | 1.00 | 54.19 |
| 2 | CG1 | VAL | A | 1 | −2.631 | 78.371 | 156.184 | 1.00 | 159.57 |
| 3 | CG2 | VAL | A | 1 | −3.131 | 76.460 | 157.704 | 1.00 | 132.31 |
| 4 | C | VAL | A | 1 | −2.948 | 80.258 | 158.492 | 1.00 | 178.97 |
| 5 | O | VAL | A | 1 | −2.487 | 80.838 | 157.504 | 1.00 | 201.24 |
| 6 | N | VAL | A | 1 | −3.255 | 78.193 | 159.967 | 1.00 | 154.76 |
| 7 | CA | VAL | A | 1 | −2.715 | 78.740 | 158.688 | 1.00 | 168.39 |
| 8 | N | PRO | A | 2 | −3.652 | 80.926 | 159.432 | 1.00 | 162.09 |
| 9 | CD | PRO | A | 2 | −4.400 | 80.420 | 160.599 | 1.00 | 80.92 |
| 10 | CA | PRO | A | 2 | −3.883 | 82.370 | 159.264 | 1.00 | 154.66 |
| 11 | CB | PRO | A | 2 | −5.040 | 82.635 | 160.223 | 1.00 | 150.00 |
| 12 | CG | PRO | A | 2 | −4.741 | 81.687 | 161.341 | 1.00 | 74.00 |
| 13 | C | PRO | A | 2 | −2.659 | 83.238 | 159.588 | 1.00 | 157.76 |
| 14 | O | PRO | A | 2 | −1.561 | 82.723 | 159.805 | 1.00 | 153.79 |
| 15 | N | GLN | A | 3 | −2.850 | 84.557 | 159.604 | 1.00 | 167.42 |
| 16 | CA | GLN | A | 3 | −1.767 | 85.480 | 159.940 | 1.00 | 118.16 |
| 17 | CB | GLN | A | 3 | −2.084 | 86.902 | 159.460 | 1.00 | 89.25 |
| 18 | CG | GLN | A | 3 | −1.705 | 87.173 | 158.009 | 1.00 | 165.02 |
| 19 | CD | GLN | A | 3 | −2.117 | 88.561 | 157.535 | 1.00 | 182.69 |
| 20 | OE1 | GLN | A | 3 | −1.725 | 89.570 | 158.120 | 1.00 | 146.97 |
| 21 | NE2 | GLN | A | 3 | −2.908 | 88.616 | 156.462 | 1.00 | 178.25 |
| 22 | C | GLN | A | 3 | −1.604 | 85.479 | 161.457 | 1.00 | 111.63 |
| 23 | O | GLN | A | 3 | −2.582 | 85.634 | 162.192 | 1.00 | 63.56 |
| 24 | N | LYS | A | 4 | −0.370 | 85.284 | 161.916 | 1.00 | 119.89 |
| 25 | CA | LYS | A | 4 | −0.062 | 85.264 | 163.344 | 1.00 | 60.75 |
| 26 | CB | LYS | A | 4 | 1.263 | 84.535 | 163.607 | 1.00 | 99.96 |
| 27 | CG | LYS | A | 4 | 1.320 | 83.103 | 163.084 | 1.00 | 192.75 |
| 28 | CD | LYS | A | 4 | 2.645 | 82.417 | 163.434 | 1.00 | 187.23 |
| 29 | CE | LYS | A | 4 | 2.670 | 80.974 | 162.925 | 1.00 | 187.05 |
| 30 | NZ | LYS | A | 4 | 3.932 | 80.256 | 163.268 | 1.00 | 160.49 |
| 31 | C | LYS | A | 4 | 0.069 | 86.705 | 163.805 | 1.00 | 68.75 |
| 32 | O | LYS | A | 4 | 0.179 | 87.615 | 162.990 | 1.00 | 95.41 |
| 33 | N | PRO | A | 5 | 0.051 | 86.938 | 165.121 | 1.00 | 28.76 |
| 34 | CD | PRO | A | 5 | −0.398 | 86.034 | 166.189 | 1.00 | 61.76 |
| 35 | CA | PRO | A | 5 | 0.176 | 88.304 | 165.632 | 1.00 | 48.28 |
| 36 | CB | PRO | A | 5 | −0.576 | 88.231 | 166.949 | 1.00 | 43.06 |
| 37 | CG | PRO | A | 5 | −0.226 | 86.882 | 167.417 | 1.00 | 11.05 |
| 38 | C | PRO | A | 5 | 1.638 | 88.734 | 165.804 | 1.00 | 63.30 |
| 39 | O | PRO | A | 5 | 2.469 | 87.967 | 166.293 | 1.00 | 54.45 |
| 40 | N | LYS | A | 6 | 1.944 | 89.961 | 165.388 | 1.00 | 63.56 |
| 41 | CA | LYS | A | 6 | 3.304 | 90.497 | 165.470 | 1.00 | 95.51 |
| 42 | CB | LYS | A | 6 | 3.647 | 91.212 | 164.151 | 1.00 | 110.28 |
| 43 | CG | LYS | A | 6 | 5.083 | 91.745 | 164.040 | 1.00 | 190.76 |
| 44 | CD | LYS | A | 6 | 6.120 | 90.634 | 163.856 | 1.00 | 206.32 |
| 45 | CE | LYS | A | 6 | 7.533 | 91.195 | 163.662 | 1.00 | 184.83 |
| 46 | NZ | LYS | A | 6 | 7.695 | 91.956 | 162.385 | 1.00 | 173.73 |
| 47 | C | LYS | A | 6 | 3.467 | 91.461 | 166.658 | 1.00 | 86.34 |
| 48 | O | LYS | A | 6 | 2.486 | 92.000 | 167.156 | 1.00 | 52.28 |
| 49 | N | VAL | A | 7 | 4.706 | 91.655 | 167.118 | 1.00 | 90.20 |
| 50 | CA | VAL | A | 7 | 5.010 | 92.561 | 168.234 | 1.00 | 22.49 |
| 51 | CB | VAL | A | 7 | 5.718 | 91.859 | 169.380 | 1.00 | 13.79 |
| 52 | CG1 | VAL | A | 7 | 4.920 | 92.017 | 170.651 | 1.00 | 31.43 |
| 53 | CG2 | VAL | A | 7 | 5.937 | 90.401 | 169.033 | 1.00 | 92.97 |
| 54 | C | VAL | A | 7 | 5.970 | 93.614 | 167.743 | 1.00 | 46.84 |
| 55 | O | VAL | A | 7 | 6.960 | 93.293 | 167.084 | 1.00 | 64.82 |
| 56 | N | SER | A | 8 | 5.680 | 94.867 | 168.065 | 1.00 | 36.46 |
| 57 | CA | SER | A | 8 | 6.527 | 95.968 | 167.652 | 1.00 | 64.14 |
| 58 | CB | SER | A | 8 | 5.721 | 96.962 | 166.816 | 1.00 | 31.16 |
| 59 | OG | SER | A | 8 | 4.731 | 97.609 | 167.600 | 1.00 | 112.57 |
| 60 | C | SER | A | 8 | 7.100 | 96.651 | 168.898 | 1.00 | 70.84 |
| 61 | O | SER | A | 8 | 6.467 | 96.688 | 169.957 | 1.00 | 65.38 |
| 62 | N | LEU | A | 9 | 8.311 | 97.173 | 168.772 | 1.00 | 34.20 |
| 63 | CA | LEU | A | 9 | 8.962 | 97.844 | 169.877 | 1.00 | 17.68 |
| 64 | CB | LEU | A | 9 | 10.335 | 97.254 | 170.082 | 1.00 | 35.73 |
| 65 | CG | LEU | A | 9 | 10.470 | 95.929 | 170.799 | 1.00 | 5.42 |
| 66 | CD1 | LEU | A | 9 | 11.720 | 95.218 | 170.359 | 1.00 | 46.16 |
| 67 | CD2 | LEU | A | 9 | 10.552 | 96.200 | 172.244 | 1.00 | 38.43 |
| 68 | C | LEU | A | 9 | 9.127 | 99.312 | 169.564 | 1.00 | 46.19 |
| 69 | O | LEU | A | 9 | 9.420 | 99.680 | 168.438 | 1.00 | 59.24 |
| 70 | N | ASN | A | 10 | 8.948 | 100.161 | 170.558 | 1.00 | 40.05 |
| 71 | CA | ASN | A | 10 | 9.130 | 101.576 | 170.325 | 1.00 | 38.01 |
| 72 | CB | ASN | A | 10 | 7.815 | 102.221 | 169.923 | 1.00 | 60.50 |
| 73 | CG | ASN | A | 10 | 7.972 | 103.675 | 169.566 | 1.00 | 65.72 |
| 74 | OD1 | ASN | A | 10 | 7.555 | 104.551 | 170.319 | 1.00 | 78.78 |
| 75 | ND2 | ASN | A | 10 | 8.588 | 103.946 | 168.419 | 1.00 | 85.11 |
| 76 | C | ASN | A | 10 | 9.683 | 102.237 | 171.567 | 1.00 | 46.63 |
| 77 | O | ASN | A | 10 | 8.989 | 102.372 | 172.570 | 1.00 | 38.10 |
| 78 | N | PRO | A | 11 | 10.952 | 102.661 | 171.513 | 1.00 | 36.23 |
| 79 | CD | PRO | A | 11 | 11.474 | 103.662 | 172.446 | 1.00 | 18.87 |
| 80 | CA | PRO | A | 11 | 11.852 | 102.543 | 170.367 | 1.00 | 23.13 |
| 81 | CB | PRO | A | 11 | 12.980 | 103.476 | 170.747 | 1.00 | 49.98 |
| 82 | CG | PRO | A | 11 | 12.266 | 104.514 | 171.534 | 1.00 | 45.32 |
| 83 | C | PRO | A | 11 | 12.331 | 101.123 | 170.053 | 1.00 | 52.44 |
| 84 | O | PRO | A | 11 | 12.575 | 100.322 | 170.964 | 1.00 | 41.38 |
| 85 | N | PRO | A | 12 | 12.530 | 100.832 | 168.752 | 1.00 | 25.66 |
| 86 | CD | PRO | A | 12 | 12.912 | 101.992 | 167.931 | 1.00 | 5.42 |
| 87 | CA | PRO | A | 12 | 12.961 | 99.600 | 168.075 | 1.00 | 20.64 |
| 88 | CB | PRO | A | 12 | 13.691 | 100.118 | 166.845 | 1.00 | 15.38 |
| 89 | CG | PRO | A | 12 | 13.032 | 101.395 | 166.573 | 1.00 | 36.22 |
| 90 | C | PRO | A | 12 | 13.858 | 98.688 | 168.875 | 1.00 | 26.86 |
| 91 | O | PRO | A | 12 | 13.653 | 97.480 | 168.946 | 1.00 | 75.83 |
| 92 | N | TRP | A | 13 | 14.881 | 99.297 | 169.445 | 1.00 | 51.05 |
| 93 | CA | TRP | A | 13 | 15.898 | 98.623 | 170.226 | 1.00 | 46.59 |
| 94 | CB | TRP | A | 13 | 16.675 | 99.690 | 170.966 | 1.00 | 5.42 |
| 95 | CG | TRP | A | 13 | 16.638 | 100.993 | 170.234 | 1.00 | 17.98 |
| 96 | CD2 | TRP | A | 13 | 16.993 | 101.221 | 168.870 | 1.00 | 11.54 |
| 97 | CE2 | TRP | A | 13 | 16.869 | 102.602 | 168.631 | 1.00 | 36.13 |
| 98 | CE3 | TRP | A | 13 | 17.413 | 100.392 | 167.826 | 1.00 | 16.34 |
| 99 | CD1 | TRP | A | 13 | 16.310 | 102.211 | 170.749 | 1.00 | 79.53 |
| 100 | NE1 | TRP | A | 13 | 16.446 | 103.183 | 169.797 | 1.00 | 12.94 |
| 101 | CZ2 | TRP | A | 13 | 17.158 | 103.178 | 167.391 | 1.00 | 5.42 |
| 102 | CZ3 | TRP | A | 13 | 17.698 | 100.965 | 166.596 | 1.00 | 58.81 |
| 103 | CH2 | TRP | A | 13 | 17.572 | 102.346 | 166.393 | 1.00 | 14.55 |
| 104 | C | TRP | A | 13 | 15.357 | 97.588 | 171.200 | 1.00 | 53.67 |
| 105 | O | TRP | A | 13 | 14.615 | 97.929 | 172.116 | 1.00 | 40.75 |
| 106 | N | ASN | A | 14 | 15.726 | 96.326 | 171.003 | 1.00 | 40.23 |
| 107 | CA | ASN | A | 14 | 15.272 | 95.289 | 171.905 | 1.00 | 39.68 |
| 108 | CB | ASN | A | 14 | 14.910 | 94.012 | 171.148 | 1.00 | 55.19 |
| 109 | CG | ASN | A | 14 | 15.994 | 93.556 | 170.225 | 1.00 | 52.50 |
| 110 | OD1 | ASN | A | 14 | 17.116 | 93.309 | 170.653 | 1.00 | 34.32 |
| 111 | ND2 | ASN | A | 14 | 15.668 | 93.437 | 168.940 | 1.00 | 84.53 |
| 112 | C | ASN | A | 14 | 16.328 | 95.017 | 172.956 | 1.00 | 39.79 |
| 113 | O | ASN | A | 14 | 16.232 | 94.049 | 173.704 | 1.00 | 68.24 |
| 114 | N | ARG | A | 15 | 17.344 | 95.876 | 172.992 | 1.00 | 42.06 |
| 115 | CA | ARG | A | 15 | 18.420 | 95.815 | 173.989 | 1.00 | 51.52 |
| 116 | CB | ARG | A | 15 | 19.738 | 95.306 | 173.392 | 1.00 | 16.41 |
| 117 | CG | ARG | A | 15 | 20.077 | 95.790 | 172.006 | 1.00 | 43.90 |
| 118 | CD | ARG | A | 15 | 21.494 | 95.348 | 171.646 | 1.00 | 69.87 |
| 119 | NE | ARG | A | 15 | 21.733 | 93.931 | 171.916 | 1.00 | 49.77 |
| 120 | CZ | ARG | A | 15 | 22.922 | 93.426 | 172.230 | 1.00 | 53.72 |
| 121 | NH1 | ARG | A | 15 | 23.985 | 94.212 | 172.321 | 1.00 | 38.62 |
| 122 | NH2 | ARG | A | 15 | 23.050 | 92.129 | 172.455 | 1.00 | 109.67 |
| 123 | C | ARG | A | 15 | 18.581 | 97.235 | 174.499 | 1.00 | 19.10 |
| 124 | O | ARG | A | 15 | 18.822 | 98.147 | 173.725 | 1.00 | 10.96 |
| 125 | N | ILE | A | 16 | 18.440 | 97.420 | 175.802 | 1.00 | 22.57 |
| 126 | CA | ILE | A | 16 | 18.502 | 98.752 | 176.385 | 1.00 | 22.94 |
| 127 | CB | ILE | A | 16 | 17.101 | 99.298 | 176.601 | 1.00 | 32.81 |
| 128 | CG2 | ILE | A | 16 | 16.463 | 99.668 | 175.286 | 1.00 | 35.27 |
| 129 | CG1 | ILE | A | 16 | 16.283 | 98.250 | 177.349 | 1.00 | 10.08 |
| 130 | CD1 | ILE | A | 16 | 14.931 | 98.711 | 177.716 | 1.00 | 45.93 |
| 131 | C | ILE | A | 16 | 19.170 | 98.826 | 177.745 | 1.00 | 47.61 |
| 132 | O | ILE | A | 16 | 19.175 | 97.854 | 178.495 | 1.00 | 52.58 |
| 133 | N | PHE | A | 17 | 19.693 | 100.003 | 178.070 | 1.00 | 19.08 |
| 134 | CA | PHE | A | 17 | 20.332 | 100.224 | 179.361 | 1.00 | 30.02 |
| 135 | CB | PHE | A | 17 | 20.977 | 101.603 | 179.405 | 1.00 | 34.47 |
| 136 | CG | PHE | A | 17 | 22.216 | 101.709 | 178.604 | 1.00 | 34.70 |
| 137 | CD1 | PHE | A | 17 | 22.493 | 102.868 | 177.889 | 1.00 | 48.32 |
| 138 | CD2 | PHE | A | 17 | 23.105 | 100.649 | 178.544 | 1.00 | 11.47 |
| 139 | CE1 | PHE | A | 17 | 23.633 | 102.972 | 177.121 | 1.00 | 16.41 |
| 140 | CE2 | PHE | A | 17 | 24.246 | 100.739 | 177.782 | 1.00 | 40.21 |
| 141 | CZ | PHE | A | 17 | 24.513 | 101.904 | 177.065 | 1.00 | 105.94 |
| 142 | C | PHE | A | 17 | 19.282 | 100.153 | 180.456 | 1.00 | 37.78 |
| 143 | O | PHE | A | 17 | 18.146 | 100.564 | 180.256 | 1.00 | 17.05 |
| 144 | N | LYS | A | 18 | 19.661 | 99.662 | 181.624 | 1.00 | 5.42 |
| 145 | CA | LYS | A | 18 | 18.702 | 99.583 | 182.696 | 1.00 | 38.72 |
| 146 | CB | LYS | A | 18 | 19.318 | 98.921 | 183.931 | 1.00 | 15.58 |
| 147 | CG | LYS | A | 18 | 19.768 | 99.862 | 185.000 | 1.00 | 22.71 |
| 148 | CD | LYS | A | 18 | 20.290 | 99.109 | 186.226 | 1.00 | 39.40 |
| 149 | CE | LYS | A | 18 | 19.181 | 98.710 | 187.181 | 1.00 | 57.95 |
| 150 | NZ | LYS | A | 18 | 19.692 | 98.586 | 188.585 | 1.00 | 58.03 |
| 151 | C | LYS | A | 18 | 18.213 | 100.972 | 183.034 | 1.00 | 9.14 |
| 152 | O | LYS | A | 18 | 18.976 | 101.919 | 183.006 | 1.00 | 14.56 |
| 153 | N | GLY | A | 19 | 16.928 | 101.071 | 183.353 | 1.00 | 48.65 |
| 154 | CA | GLY | A | 19 | 16.338 | 102.342 | 183.702 | 1.00 | 37.66 |
| 155 | C | GLY | A | 19 | 15.760 | 103.020 | 182.487 | 1.00 | 11.50 |
| 156 | O | GLY | A | 19 | 15.196 | 104.106 | 182.580 | 1.00 | 82.09 |
| 157 | N | GLU | A | 20 | 15.916 | 102.389 | 181.332 | 1.00 | 51.25 |
| 158 | CA | GLU | A | 20 | 15.390 | 102.959 | 180.101 | 1.00 | 39.98 |
| 159 | CB | GLU | A | 20 | 16.245 | 102.547 | 178.901 | 1.00 | 61.38 |
| 160 | CG | GLU | A | 20 | 17.645 | 103.141 | 178.937 | 1.00 | 107.42 |
| 161 | CD | GLU | A | 20 | 18.374 | 103.073 | 177.608 | 1.00 | 75.17 |
| 162 | OE1 | GLU | A | 20 | 19.490 | 103.620 | 177.537 | 1.00 | 70.97 |
| 163 | OE2 | GLU | A | 20 | 17.847 | 102.483 | 176.639 | 1.00 | 72.76 |
| 164 | C | GLU | A | 20 | 13.950 | 102.532 | 179.893 | 1.00 | 47.68 |
| 165 | O | GLU | A | 20 | 13.449 | 101.624 | 180.565 | 1.00 | 16.47 |
| 166 | N | ASN | A | 21 | 13.280 | 103.200 | 178.964 | 1.00 | 51.83 |
| 167 | CA | ASN | A | 21 | 11.885 | 102.910 | 178.692 | 1.00 | 39.65 |
| 168 | CB | ASN | A | 21 | 11.057 | 104.195 | 178.786 | 1.00 | 36.44 |
| 169 | CG | ASN | A | 21 | 11.008 | 104.762 | 180.191 | 1.00 | 24.19 |
| 170 | OD1 | ASN | A | 21 | 10.954 | 104.009 | 181.164 | 1.00 | 25.70 |
| 171 | ND2 | ASN | A | 21 | 11.002 | 106.089 | 180.298 | 1.00 | 72.05 |
| 172 | C | ASN | A | 21 | 11.653 | 102.271 | 177.340 | 1.00 | 14.38 |
| 173 | O | ASN | A | 21 | 12.362 | 102.554 | 176.384 | 1.00 | 96.93 |
| 174 | N | VAL | A | 22 | 10.651 | 101.405 | 177.270 | 1.00 | 55.87 |
| 175 | CA | VAL | A | 22 | 10.305 | 100.748 | 176.023 | 1.00 | 39.97 |
| 176 | CB | VAL | A | 22 | 11.168 | 99.525 | 175.769 | 1.00 | 23.77 |
| 177 | CG1 | VAL | A | 22 | 10.880 | 98.461 | 176.789 | 1.00 | 24.69 |
| 178 | CG2 | VAL | A | 22 | 10.896 | 99.013 | 174.395 | 1.00 | 10.67 |
| 179 | C | VAL | A | 22 | 8.861 | 100.308 | 176.057 | 1.00 | 39.52 |
| 180 | O | VAL | A | 22 | 8.299 | 100.143 | 177.134 | 1.00 | 50.04 |
| 181 | N | THR | A | 23 | 8.273 | 100.106 | 174.879 | 1.00 | 35.08 |
| 182 | CA | THR | A | 23 | 6.876 | 99.689 | 174.758 | 1.00 | 50.38 |
| 183 | CB | THR | A | 23 | 5.982 | 100.883 | 174.356 | 1.00 | 15.46 |
| 184 | OG1 | THR | A | 23 | 5.325 | 101.397 | 175.515 | 1.00 | 51.09 |
| 185 | CG2 | THR | A | 23 | 4.944 | 100.470 | 173.342 | 1.00 | 53.00 |
| 186 | C | THR | A | 23 | 6.638 | 98.564 | 173.758 | 1.00 | 24.12 |
| 187 | O | THR | A | 23 | 7.121 | 98.601 | 172.629 | 1.00 | 33.75 |
| 188 | N | LEU | A | 24 | 5.869 | 97.567 | 174.170 | 1.00 | 32.66 |
| 189 | CA | LEU | A | 24 | 5.565 | 96.463 | 173.278 | 1.00 | 45.63 |
| 190 | CB | LEU | A | 24 | 5.754 | 95.119 | 173.987 | 1.00 | 23.83 |
| 191 | CG | LEU | A | 24 | 7.072 | 94.939 | 174.739 | 1.00 | 28.78 |
| 192 | CD1 | LEU | A | 24 | 7.381 | 93.488 | 174.933 | 1.00 | 19.89 |
| 193 | CD2 | LEU | A | 24 | 8.159 | 95.572 | 173.969 | 1.00 | 5.42 |
| 194 | C | LEU | A | 24 | 4.128 | 96.607 | 172.822 | 1.00 | 44.86 |
| 195 | O | LEU | A | 24 | 3.248 | 96.866 | 173.635 | 1.00 | 52.63 |
| 196 | N | THR | A | 25 | 3.895 | 96.444 | 171.523 | 1.00 | 52.39 |
| 197 | CA | THR | A | 25 | 2.554 | 96.550 | 170.965 | 1.00 | 46.08 |
| 198 | CB | THR | A | 25 | 2.454 | 97.761 | 170.049 | 1.00 | 24.50 |
| 199 | OG1 | THR | A | 25 | 3.088 | 98.884 | 170.673 | 1.00 | 62.13 |
| 200 | CG2 | THR | A | 25 | 1.016 | 98.098 | 169.807 | 1.00 | 77.17 |
| 201 | C | THR | A | 25 | 2.233 | 95.282 | 170.174 | 1.00 | 59.55 |
| 202 | O | THR | A | 25 | 3.120 | 94.707 | 169.542 | 1.00 | 36.44 |
| 203 | N | CYS | A | 26 | 0.970 | 94.852 | 170.215 | 1.00 | 59.40 |
| 204 | CA | CYS | A | 26 | 0.520 | 93.642 | 169.525 | 1.00 | 38.53 |
| 205 | C | CYS | A | 26 | −0.343 | 94.009 | 168.318 | 1.00 | 42.63 |
| 206 | O | CYS | A | 26 | −1.322 | 94.734 | 168.447 | 1.00 | 77.67 |
| 207 | CB | CYS | A | 26 | −0.256 | 92.757 | 170.514 | 1.00 | 31.91 |
| 208 | SG | CYS | A | 26 | −0.296 | 90.939 | 170.208 | 1.00 | 112.14 |
| 209 | N | ASN | A | 27 | 0.083 | 93.514 | 167.154 | 1.00 | 114.86 |
| 210 | CA | ASN | A | 27 | −0.506 | 93.677 | 165.805 | 1.00 | 124.26 |
| 211 | CB | ASN | A | 27 | −0.765 | 92.286 | 165.217 | 1.00 | 138.85 |
| 212 | CG | ASN | A | 27 | −0.588 | 92.249 | 163.708 | 1.00 | 187.97 |
| 213 | OD1 | ASN | A | 27 | −0.071 | 93.196 | 163.106 | 1.00 | 180.59 |
| 214 | ND2 | ASN | A | 27 | −0.999 | 91.145 | 163.090 | 1.00 | 211.12 |
| 215 | C | ASN | A | 27 | −1.717 | 94.563 | 165.469 | 1.00 | 69.87 |
| 216 | O | ASN | A | 27 | −2.604 | 94.788 | 166.278 | 1.00 | 99.87 |
| 217 | N | GLY | A | 28 | −1.737 | 95.043 | 164.228 | 1.00 | 93.93 |
| 218 | CA | GLY | A | 28 | −2.818 | 95.887 | 163.752 | 1.00 | 33.38 |
| 219 | C | GLY | A | 28 | −3.811 | 95.070 | 162.949 | 1.00 | 71.27 |
| 220 | O | GLY | A | 28 | −4.658 | 95.611 | 162.243 | 1.00 | 61.63 |
| 221 | N | ASN | A | 29 | −3.686 | 93.752 | 163.064 | 1.00 | 116.52 |
| 222 | CA | ASN | A | 29 | −4.550 | 92.783 | 162.388 | 1.00 | 70.71 |
| 223 | CB | ASN | A | 29 | −3.729 | 91.533 | 162.062 | 1.00 | 121.95 |
| 224 | CG | ASN | A | 29 | −4.164 | 90.852 | 160.783 | 1.00 | 150.49 |
| 225 | OD1 | ASN | A | 29 | −4.247 | 91.480 | 159.727 | 1.00 | 172.84 |
| 226 | ND2 | ASN | A | 29 | −4.428 | 89.550 | 160.868 | 1.00 | 174.68 |
| 227 | C | ASN | A | 29 | −5.658 | 92.466 | 163.405 | 1.00 | 97.53 |
| 228 | O | ASN | A | 29 | −6.252 | 91.389 | 163.421 | 1.00 | 106.16 |
| 229 | N | ASN | A | 30 | −5.886 | 93.448 | 164.265 | 1.00 | 53.59 |
| 230 | CA | ASN | A | 30 | −6.878 | 93.432 | 165.332 | 1.00 | 80.30 |
| 231 | CB | ASN | A | 30 | −6.317 | 92.689 | 166.558 | 1.00 | 85.88 |
| 232 | CG | ASN | A | 30 | −7.040 | 93.041 | 167.875 | 1.00 | 109.70 |
| 233 | OD1 | ASN | A | 30 | −8.256 | 92.883 | 168.011 | 1.00 | 70.68 |
| 234 | ND2 | ASN | A | 30 | −6.272 | 93.507 | 168.851 | 1.00 | 35.68 |
| 235 | C | ASN | A | 30 | −7.041 | 94.917 | 165.623 | 1.00 | 78.22 |
| 236 | O | ASN | A | 30 | −6.772 | 95.363 | 166.729 | 1.00 | 51.75 |
| 237 | N | PHE | A | 31 | −7.493 | 95.667 | 164.617 | 1.00 | 89.72 |
| 238 | CA | PHE | A | 31 | −7.629 | 97.125 | 164.709 | 1.00 | 96.61 |
| 239 | CB | PHE | A | 31 | −7.900 | 97.716 | 163.320 | 1.00 | 115.42 |
| 240 | CG | PHE | A | 31 | −7.680 | 99.211 | 163.242 | 1.00 | 120.39 |
| 241 | CD1 | PHE | A | 31 | −6.403 | 99.745 | 163.398 | 1.00 | 112.19 |
| 242 | CD2 | PHE | A | 31 | −8.746 | 100.083 | 163.023 | 1.00 | 119.97 |
| 243 | CE1 | PHE | A | 31 | −6.190 | 101.121 | 163.339 | 1.00 | 72.84 |
| 244 | CE2 | PHE | A | 31 | −8.544 | 101.460 | 162.962 | 1.00 | 60.99 |
| 245 | CZ | PHE | A | 31 | −7.262 | 101.978 | 163.122 | 1.00 | 105.12 |
| 246 | C | PHE | A | 31 | −8.585 | 97.783 | 165.707 | 1.00 | 94.85 |
| 247 | O | PHE | A | 31 | −8.131 | 98.494 | 166.601 | 1.00 | 127.53 |
| 248 | N | PHE | A | 32 | −9.894 | 97.596 | 165.564 | 1.00 | 91.11 |
| 249 | CA | PHE | A | 32 | −10.818 | 98.248 | 166.500 | 1.00 | 92.26 |
| 250 | CB | PHE | A | 32 | −12.272 | 98.103 | 166.052 | 1.00 | 93.61 |
| 251 | CG | PHE | A | 32 | −12.504 | 98.467 | 164.625 | 1.00 | 103.12 |
| 252 | CD1 | PHE | A | 32 | −12.136 | 97.591 | 163.600 | 1.00 | 99.09 |
| 253 | CD2 | PHE | A | 32 | −13.064 | 99.693 | 164.298 | 1.00 | 32.51 |
| 254 | CE1 | PHE | A | 32 | −12.320 | 97.933 | 162.268 | 1.00 | 63.07 |
| 255 | CE2 | PHE | A | 32 | −13.251 | 100.044 | 162.974 | 1.00 | 108.41 |
| 256 | CZ | PHE | A | 32 | −12.877 | 99.160 | 161.953 | 1.00 | 118.33 |
| 257 | C | PHE | A | 32 | −10.673 | 97.618 | 167.867 | 1.00 | 102.23 |
| 258 | O | PHE | A | 32 | −11.305 | 98.050 | 168.838 | 1.00 | 76.95 |
| 259 | N | GLU | A | 33 | −9.827 | 96.593 | 167.918 | 1.00 | 90.59 |
| 260 | CA | GLU | A | 33 | −9.567 | 95.837 | 169.127 | 1.00 | 58.80 |
| 261 | CB | GLU | A | 33 | −9.193 | 96.766 | 170.287 | 1.00 | 34.16 |
| 262 | CG | GLU | A | 33 | −7.709 | 97.116 | 170.319 | 1.00 | 83.37 |
| 263 | CD | GLU | A | 33 | −7.302 | 97.846 | 171.583 | 1.00 | 138.46 |
| 264 | OE1 | GLU | A | 33 | −7.822 | 97.498 | 172.666 | 1.00 | 156.92 |
| 265 | OE2 | GLU | A | 33 | −6.450 | 98.757 | 171.494 | 1.00 | 143.48 |
| 266 | C | GLU | A | 33 | −10.807 | 95.038 | 169.458 | 1.00 | 51.35 |
| 267 | O | GLU | A | 33 | −11.670 | 95.489 | 170.207 | 1.00 | 76.71 |
| 268 | N | VAL | A | 34 | −10.889 | 93.849 | 168.874 | 1.00 | 40.24 |
| 269 | CA | VAL | A | 34 | −12.018 | 92.963 | 169.092 | 1.00 | 63.51 |
| 270 | CB | VAL | A | 34 | −12.369 | 92.212 | 167.815 | 1.00 | 12.87 |
| 271 | CG1 | VAL | A | 34 | −12.724 | 93.199 | 166.743 | 1.00 | 47.66 |
| 272 | CG2 | VAL | A | 34 | −11.194 | 91.350 | 167.382 | 1.00 | 80.18 |
| 273 | C | VAL | A | 34 | −11.691 | 91.960 | 170.185 | 1.00 | 48.93 |
| 274 | O | VAL | A | 34 | −12.584 | 91.300 | 170.719 | 1.00 | 66.41 |
| 275 | N | SER | A | 35 | −10.411 | 91.840 | 170.518 | 1.00 | 40.51 |
| 276 | CA | SER | A | 35 | −10.027 | 90.913 | 171.568 | 1.00 | 79.37 |
| 277 | CB | SER | A | 35 | −9.460 | 89.642 | 170.974 | 1.00 | 33.93 |
| 278 | OG | SER | A | 35 | −8.107 | 89.851 | 170.650 | 1.00 | 44.80 |
| 279 | C | SER | A | 35 | −8.991 | 91.484 | 172.530 | 1.00 | 83.70 |
| 280 | O | SER | A | 35 | −8.097 | 92.242 | 172.139 | 1.00 | 52.41 |
| 281 | N | SER | A | 36 | −9.127 | 91.112 | 173.798 | 1.00 | 57.27 |
| 282 | CA | SER | A | 36 | −8.195 | 91.539 | 174.819 | 1.00 | 23.32 |
| 283 | CB | SER | A | 36 | −8.600 | 90.956 | 176.156 | 1.00 | 119.43 |
| 284 | OG | SER | A | 36 | −8.593 | 89.547 | 176.089 | 1.00 | 43.01 |
| 285 | C | SER | A | 36 | −6.879 | 90.929 | 174.408 | 1.00 | 49.51 |
| 286 | O | SER | A | 36 | −6.857 | 89.930 | 173.702 | 1.00 | 34.96 |
| 287 | N | THR | A | 37 | −5.780 | 91.517 | 174.854 | 1.00 | 52.38 |
| 288 | CA | THR | A | 37 | −4.466 | 90.996 | 174.516 | 1.00 | 34.52 |
| 289 | CB | THR | A | 37 | −3.501 | 92.113 | 174.181 | 1.00 | 28.99 |
| 290 | OG1 | THR | A | 37 | −4.074 | 92.965 | 173.182 | 1.00 | 95.55 |
| 291 | CG2 | THR | A | 37 | −2.211 | 91.537 | 173.682 | 1.00 | 68.24 |
| 292 | C | THR | A | 37 | −3.890 | 90.283 | 175.710 | 1.00 | 57.63 |
| 293 | O | THR | A | 37 | −4.159 | 90.662 | 176.848 | 1.00 | 34.77 |
| 294 | N | LYS | A | 38 | −3.109 | 89.241 | 175.469 | 1.00 | 28.90 |
| 295 | CA | LYS | A | 38 | −2.491 | 88.551 | 176.588 | 1.00 | 65.81 |
| 296 | CB | LYS | A | 38 | −3.111 | 87.179 | 176.799 | 1.00 | 34.97 |
| 297 | CG | LYS | A | 38 | −3.818 | 86.631 | 175.615 | 1.00 | 36.39 |
| 298 | CD | LYS | A | 38 | −5.098 | 85.965 | 176.076 | 1.00 | 85.18 |
| 299 | CE | LYS | A | 38 | −6.234 | 86.958 | 176.203 | 1.00 | 26.12 |
| 300 | NZ | LYS | A | 38 | −7.146 | 86.806 | 175.029 | 1.00 | 20.00 |
| 301 | C | LYS | A | 38 | −0.990 | 88.454 | 176.381 | 1.00 | 79.44 |
| 302 | O | LYS | A | 38 | −0.523 | 87.805 | 175.444 | 1.00 | 44.12 |
| 303 | N | TRP | A | 39 | −0.256 | 89.130 | 177.268 | 1.00 | 74.45 |
| 304 | CA | TRP | A | 39 | 1.198 | 89.204 | 177.230 | 1.00 | 12.82 |
| 305 | CB | TRP | A | 39 | 1.656 | 90.574 | 177.704 | 1.00 | 55.75 |
| 306 | CG | TRP | A | 39 | 1.180 | 91.696 | 176.875 | 1.00 | 17.73 |
| 307 | CD2 | TRP | A | 39 | 1.763 | 92.144 | 175.661 | 1.00 | 20.84 |
| 308 | CE2 | TRP | A | 39 | 0.957 | 93.195 | 175.175 | 1.00 | 24.82 |
| 309 | CE3 | TRP | A | 39 | 2.889 | 91.757 | 174.930 | 1.00 | 15.24 |
| 310 | CD1 | TRP | A | 39 | 0.069 | 92.473 | 177.082 | 1.00 | 45.58 |
| 311 | NE1 | TRP | A | 39 | −0.072 | 93.374 | 176.060 | 1.00 | 18.25 |
| 312 | CZ2 | TRP | A | 39 | 1.247 | 93.864 | 173.990 | 1.00 | 26.70 |
| 313 | CZ3 | TRP | A | 39 | 3.177 | 92.419 | 173.751 | 1.00 | 13.49 |
| 314 | CH2 | TRP | A | 39 | 2.360 | 93.463 | 173.293 | 1.00 | 70.81 |
| 315 | C | TRP | A | 39 | 1.847 | 88.166 | 178.109 | 1.00 | 55.49 |
| 316 | O | TRP | A | 39 | 1.417 | 87.945 | 179.236 | 1.00 | 33.26 |
| 317 | N | PHE | A | 40 | 2.916 | 87.566 | 177.604 | 1.00 | 28.08 |
| 318 | CA | PHE | A | 40 | 3.640 | 86.538 | 178.331 | 1.00 | 19.83 |
| 319 | CB | PHE | A | 40 | 3.527 | 85.229 | 177.574 | 1.00 | 69.82 |
| 320 | CG | PHE | A | 40 | 2.137 | 84.682 | 177.528 | 1.00 | 65.09 |
| 321 | CD1 | PHE | A | 40 | 1.608 | 84.205 | 176.338 | 1.00 | 50.58 |
| 322 | CD2 | PHE | A | 40 | 1.367 | 84.614 | 178.680 | 1.00 | 53.63 |
| 323 | CE1 | PHE | A | 40 | 0.341 | 83.673 | 176.300 | 1.00 | 68.98 |
| 324 | CE2 | PHE | A | 40 | 0.105 | 84.083 | 178.646 | 1.00 | 26.70 |
| 325 | CZ | PHE | A | 40 | −0.414 | 83.611 | 177.459 | 1.00 | 85.38 |
| 326 | C | PHE | A | 40 | 5.112 | 86.889 | 178.522 | 1.00 | 83.34 |
| 327 | O | PHE | A | 40 | 5.835 | 87.108 | 177.546 | 1.00 | 65.61 |
| 328 | N | HIS | A | 41 | 5.549 | 86.924 | 179.780 | 1.00 | 49.52 |
| 329 | CA | HIS | A | 41 | 6.929 | 87.243 | 180.121 | 1.00 | 41.84 |
| 330 | CB | HIS | A | 41 | 6.950 | 88.359 | 181.166 | 1.00 | 82.94 |
| 331 | CG | HIS | A | 41 | 8.325 | 88.832 | 181.529 | 1.00 | 109.26 |
| 332 | CD2 | HIS | A | 41 | 8.822 | 89.302 | 182.698 | 1.00 | 54.39 |
| 333 | ND1 | HIS | A | 41 | 9.361 | 88.901 | 180.618 | 1.00 | 50.58 |
| 334 | CE1 | HIS | A | 41 | 10.433 | 89.389 | 181.213 | 1.00 | 51.90 |
| 335 | NE2 | HIS | A | 41 | 10.133 | 89.642 | 182.475 | 1.00 | 80.09 |
| 336 | C | HIS | A | 41 | 7.671 | 86.006 | 180.630 | 1.00 | 66.77 |
| 337 | O | HIS | A | 41 | 7.413 | 85.511 | 181.729 | 1.00 | 49.22 |
| 338 | N | ASN | A | 42 | 8.594 | 85.513 | 179.806 | 1.00 | 91.49 |
| 339 | CA | ASN | A | 42 | 9.383 | 84.329 | 180.122 | 1.00 | 83.79 |
| 340 | CB | ASN | A | 42 | 10.313 | 84.601 | 181.315 | 1.00 | 71.40 |
| 341 | CG | ASN | A | 42 | 11.573 | 85.360 | 180.915 | 1.00 | 72.11 |
| 342 | OD1 | ASN | A | 42 | 11.498 | 86.390 | 180.243 | 1.00 | 85.40 |
| 343 | ND2 | ASN | A | 42 | 12.732 | 84.857 | 181.333 | 1.00 | 104.80 |
| 344 | C | ASN | A | 42 | 8.464 | 83.156 | 180.421 | 1.00 | 77.24 |
| 345 | O | ASN | A | 42 | 8.923 | 82.037 | 180.657 | 1.00 | 110.53 |
| 346 | N | GLY | A | 43 | 7.162 | 83.418 | 180.389 | 1.00 | 47.35 |
| 347 | CA | GLY | A | 43 | 6.201 | 82.378 | 180.662 | 1.00 | 34.83 |
| 348 | C | GLY | A | 43 | 4.909 | 82.844 | 181.300 | 1.00 | 49.78 |
| 349 | O | GLY | A | 43 | 3.855 | 82.683 | 180.707 | 1.00 | 48.66 |
| 350 | N | SER | A | 44 | 4.971 | 83.412 | 182.499 | 1.00 | 38.80 |
| 351 | CA | SER | A | 44 | 3.760 | 83.860 | 183.181 | 1.00 | 51.26 |
| 352 | CB | SER | A | 44 | 4.090 | 84.455 | 184.553 | 1.00 | 115.68 |
| 353 | OG | SER | A | 44 | 4.024 | 83.466 | 185.572 | 1.00 | 158.60 |
| 354 | C | SER | A | 44 | 2.933 | 84.859 | 182.407 | 1.00 | 57.42 |
| 355 | O | SER | A | 44 | 3.443 | 85.594 | 181.569 | 1.00 | 48.28 |
| 356 | N | LEU | A | 45 | 1.639 | 84.871 | 182.708 | 1.00 | 78.92 |
| 357 | CA | LEU | A | 45 | 0.698 | 85.769 | 182.062 | 1.00 | 43.70 |
| 358 | CB | LEU | A | 45 | −0.728 | 85.215 | 182.177 | 1.00 | 33.26 |
| 359 | CG | LEU | A | 45 | −1.810 | 86.048 | 181.475 | 1.00 | 38.67 |
| 360 | CD1 | LEU | A | 45 | −1.934 | 85.532 | 180.084 | 1.00 | 20.12 |
| 361 | CD2 | LEU | A | 45 | −3.171 | 85.962 | 182.155 | 1.00 | 16.35 |
| 362 | C | LEU | A | 45 | 0.755 | 87.134 | 182.731 | 1.00 | 45.32 |
| 363 | O | LEU | A | 45 | 0.531 | 87.243 | 183.928 | 1.00 | 46.93 |
| 364 | N | SER | A | 46 | 1.053 | 88.176 | 181.964 | 1.00 | 43.39 |
| 365 | CA | SER | A | 46 | 1.100 | 89.513 | 182.530 | 1.00 | 61.73 |
| 366 | CB | SER | A | 46 | 1.808 | 90.469 | 181.584 | 1.00 | 36.13 |
| 367 | OG | SER | A | 46 | 1.827 | 91.769 | 182.137 | 1.00 | 89.66 |
| 368 | C | SER | A | 46 | −0.316 | 90.006 | 182.778 | 1.00 | 33.11 |
| 369 | O | SER | A | 46 | −1.245 | 89.564 | 182.105 | 1.00 | 74.17 |
| 370 | N | GLU | A | 47 | −0.475 | 90.927 | 183.727 | 1.00 | 51.16 |
| 371 | CA | GLU | A | 47 | −1.794 | 91.467 | 184.059 | 1.00 | 59.94 |
| 372 | CB | GLU | A | 47 | −1.876 | 91.906 | 185.536 | 1.00 | 102.95 |
| 373 | CG | GLU | A | 47 | −1.109 | 93.176 | 185.915 | 1.00 | 167.20 |
| 374 | CD | GLU | A | 47 | −1.380 | 93.622 | 187.356 | 1.00 | 181.03 |
| 375 | OE1 | GLU | A | 47 | −2.558 | 93.869 | 187.696 | 1.00 | 179.85 |
| 376 | OE2 | GLU | A | 47 | −0.420 | 93.729 | 188.151 | 1.00 | 185.11 |
| 377 | C | GLU | A | 47 | −2.257 | 92.613 | 183.169 | 1.00 | 72.12 |
| 378 | O | GLU | A | 47 | −3.330 | 93.173 | 183.399 | 1.00 | 64.78 |
| 379 | N | GLU | A | 48 | −1.459 | 92.977 | 182.168 | 1.00 | 30.89 |
| 380 | CA | GLU | A | 48 | −1.875 | 94.033 | 181.255 | 1.00 | 56.44 |
| 381 | CB | GLU | A | 48 | −0.689 | 94.737 | 180.606 | 1.00 | 83.36 |
| 382 | CG | GLU | A | 48 | −1.099 | 95.797 | 179.581 | 1.00 | 51.54 |
| 383 | CD | GLU | A | 48 | −1.832 | 96.978 | 180.201 | 1.00 | 104.67 |
| 384 | OE1 | GLU | A | 48 | −2.168 | 96.919 | 181.403 | 1.00 | 138.97 |
| 385 | OE2 | GLU | A | 48 | −2.077 | 97.968 | 179.481 | 1.00 | 142.55 |
| 386 | C | GLU | A | 48 | −2.664 | 93.332 | 180.178 | 1.00 | 67.47 |
| 387 | O | GLU | A | 48 | −2.224 | 92.303 | 179.658 | 1.00 | 72.45 |
| 388 | N | THR | A | 49 | −3.827 | 93.874 | 179.841 | 1.00 | 44.94 |
| 389 | CA | THR | A | 49 | −4.650 | 93.249 | 178.824 | 1.00 | 53.65 |
| 390 | CB | THR | A | 49 | −6.057 | 92.937 | 179.361 | 1.00 | 38.32 |
| 391 | OG1 | THR | A | 49 | −6.717 | 94.152 | 179.731 | 1.00 | 71.69 |
| 392 | CG2 | THR | A | 49 | −5.957 | 92.031 | 180.574 | 1.00 | 68.17 |
| 393 | C | THR | A | 49 | −4.773 | 94.090 | 177.570 | 1.00 | 42.57 |
| 394 | O | THR | A | 49 | −5.323 | 93.620 | 176.572 | 1.00 | 55.30 |
| 395 | N | ASN | A | 50 | −4.244 | 95.316 | 177.618 | 1.00 | 39.30 |
| 396 | CA | ASN | A | 50 | −4.308 | 96.238 | 176.481 | 1.00 | 35.89 |
| 397 | CB | ASN | A | 50 | −4.131 | 97.680 | 176.959 | 1.00 | 40.10 |
| 398 | CG | ASN | A | 50 | −5.248 | 98.123 | 177.888 | 1.00 | 104.98 |
| 399 | OD1 | ASN | A | 50 | −6.425 | 97.966 | 177.566 | 1.00 | 104.75 |
| 400 | ND2 | ASN | A | 50 | −4.888 | 98.684 | 179.044 | 1.00 | 117.05 |
| 401 | C | ASN | A | 50 | −3.331 | 95.925 | 175.343 | 1.00 | 40.37 |
| 402 | O | ASN | A | 50 | −2.419 | 95.117 | 175.484 | 1.00 | 38.49 |
| 403 | N | SER | A | 51 | −3.545 | 96.576 | 174.208 | 1.00 | 50.20 |
| 404 | CA | SER | A | 51 | −2.751 | 96.358 | 173.012 | 1.00 | 23.03 |
| 405 | CB | SER | A | 51 | −3.377 | 97.148 | 171.867 | 1.00 | 91.07 |
| 406 | OG | SER | A | 51 | −2.997 | 96.611 | 170.612 | 1.00 | 171.49 |
| 407 | C | SER | A | 51 | −1.259 | 96.682 | 173.121 | 1.00 | 69.60 |
| 408 | O | SER | A | 51 | −0.437 | 96.164 | 172.360 | 1.00 | 46.29 |
| 409 | N | SER | A | 52 | −0.896 | 97.547 | 174.055 | 1.00 | 45.59 |
| 410 | CA | SER | A | 52 | 0.504 | 97.893 | 174.211 | 1.00 | 54.59 |
| 411 | CB | SER | A | 52 | 0.768 | 99.315 | 173.715 | 1.00 | 8.18 |
| 412 | OG | SER | A | 52 | −0.152 | 100.219 | 174.304 | 1.00 | 136.82 |
| 413 | C | SER | A | 52 | 0.905 | 97.753 | 175.661 | 1.00 | 27.90 |
| 414 | O | SER | A | 52 | 0.190 | 98.183 | 176.556 | 1.00 | 44.58 |
| 415 | N | LEU | A | 53 | 2.053 | 97.118 | 175.870 | 1.00 | 63.94 |
| 416 | CA | LEU | A | 53 | 2.613 | 96.882 | 177.188 | 1.00 | 46.87 |
| 417 | CB | LEU | A | 53 | 3.060 | 95.430 | 177.306 | 1.00 | 46.27 |
| 418 | CG | LEU | A | 53 | 3.830 | 95.066 | 178.569 | 1.00 | 30.42 |
| 419 | CD1 | LEU | A | 53 | 2.999 | 95.372 | 179.782 | 1.00 | 91.21 |
| 420 | CD2 | LEU | A | 53 | 4.172 | 93.618 | 178.534 | 1.00 | 12.37 |
| 421 | C | LEU | A | 53 | 3.811 | 97.789 | 177.394 | 1.00 | 56.10 |
| 422 | O | LEU | A | 53 | 4.693 | 97.864 | 176.539 | 1.00 | 60.76 |
| 423 | N | ASN | A | 54 | 3.841 | 98.474 | 178.529 | 1.00 | 47.36 |
| 424 | CA | ASN | A | 54 | 4.937 | 99.378 | 178.841 | 1.00 | 46.29 |
| 425 | CB | ASN | A | 54 | 4.403 | 100.640 | 179.506 | 1.00 | 42.78 |
| 426 | CG | ASN | A | 54 | 3.436 | 101.383 | 178.631 | 1.00 | 41.09 |
| 427 | OD1 | ASN | A | 54 | 3.786 | 101.807 | 177.538 | 1.00 | 49.17 |
| 428 | ND2 | ASN | A | 54 | 2.207 | 101.545 | 179.104 | 1.00 | 84.48 |
| 429 | C | ASN | A | 54 | 5.976 | 98.749 | 179.753 | 1.00 | 33.11 |
| 430 | O | ASN | A | 54 | 5.771 | 97.691 | 180.331 | 1.00 | 62.93 |
| 431 | N | ILE | A | 55 | 7.106 | 99.422 | 179.867 | 1.00 | 61.10 |
| 432 | CA | ILE | A | 55 | 8.189 | 98.981 | 180.719 | 1.00 | 47.90 |
| 433 | CB | ILE | A | 55 | 9.217 | 98.157 | 179.946 | 1.00 | 13.22 |
| 434 | CG2 | ILE | A | 55 | 10.449 | 97.922 | 180.795 | 1.00 | 78.62 |
| 435 | CG1 | ILE | A | 55 | 8.619 | 96.807 | 179.586 | 1.00 | 27.07 |
| 436 | CD1 | ILE | A | 55 | 9.649 | 95.826 | 179.074 | 1.00 | 30.03 |
| 437 | C | ILE | A | 55 | 8.829 | 100.267 | 181.189 | 1.00 | 83.99 |
| 438 | O | ILE | A | 55 | 9.222 | 101.101 | 180.371 | 1.00 | 75.43 |
| 439 | N | VAL | A | 56 | 8.923 | 100.443 | 182.501 | 1.00 | 33.41 |
| 440 | CA | VAL | A | 56 | 9.505 | 101.660 | 183.024 | 1.00 | 49.06 |
| 441 | CB | VAL | A | 56 | 8.444 | 102.453 | 183.758 | 1.00 | 15.18 |
| 442 | CG1 | VAL | A | 56 | 9.046 | 103.714 | 184.329 | 1.00 | 108.22 |
| 443 | CG2 | VAL | A | 56 | 7.328 | 102.792 | 182.789 | 1.00 | 29.46 |
| 444 | C | VAL | A | 56 | 10.706 | 101.421 | 183.923 | 1.00 | 69.57 |
| 445 | O | VAL | A | 56 | 10.757 | 100.422 | 184.648 | 1.00 | 49.84 |
| 446 | N | ASN | A | 57 | 11.664 | 102.348 | 183.872 | 1.00 | 28.08 |
| 447 | CA | ASN | A | 57 | 12.887 | 102.233 | 184.656 | 1.00 | 76.91 |
| 448 | CB | ASN | A | 57 | 12.673 | 102.732 | 186.090 | 1.00 | 41.01 |
| 449 | CG | ASN | A | 57 | 12.080 | 104.137 | 186.133 | 1.00 | 126.97 |
| 450 | OD1 | ASN | A | 57 | 12.275 | 104.939 | 185.212 | 1.00 | 85.74 |
| 451 | ND2 | ASN | A | 57 | 11.359 | 104.444 | 187.211 | 1.00 | 113.52 |
| 452 | C | ASN | A | 57 | 13.219 | 100.756 | 184.636 | 1.00 | 51.64 |
| 453 | O | ASN | A | 57 | 13.382 | 100.110 | 185.669 | 1.00 | 51.28 |
| 454 | N | ALA | A | 58 | 13.294 | 100.237 | 183.419 | 1.00 | 5.42 |
| 455 | CA | ALA | A | 58 | 13.562 | 98.838 | 183.185 | 1.00 | 28.33 |
| 456 | CB | ALA | A | 58 | 13.971 | 98.636 | 181.763 | 1.00 | 26.01 |
| 457 | C | ALA | A | 58 | 14.604 | 98.236 | 184.083 | 1.00 | 26.51 |
| 458 | O | ALA | A | 58 | 15.769 | 98.571 | 183.983 | 1.00 | 45.62 |
| 459 | N | LYS | A | 59 | 14.182 | 97.336 | 184.962 | 1.00 | 54.38 |
| 460 | CA | LYS | A | 59 | 15.114 | 96.654 | 185.838 | 1.00 | 54.01 |
| 461 | CB | LYS | A | 59 | 14.388 | 96.159 | 187.080 | 1.00 | 49.09 |
| 462 | CG | LYS | A | 59 | 15.301 | 95.437 | 188.061 | 1.00 | 140.01 |
| 463 | CD | LYS | A | 59 | 16.432 | 96.316 | 188.603 | 1.00 | 80.75 |
| 464 | CE | LYS | A | 59 | 17.313 | 95.516 | 189.541 | 1.00 | 71.72 |
| 465 | NZ | LYS | A | 59 | 17.864 | 94.316 | 188.836 | 1.00 | 63.79 |
| 466 | C | LYS | A | 59 | 15.681 | 95.481 | 185.031 | 1.00 | 38.80 |
| 467 | O | LYS | A | 59 | 15.234 | 95.229 | 183.920 | 1.00 | 40.61 |
| 468 | N | PHE | A | 60 | 16.673 | 94.771 | 185.549 | 1.00 | 36.75 |
| 469 | CA | PHE | A | 60 | 17.207 | 93.658 | 184.776 | 1.00 | 26.24 |
| 470 | CB | PHE | A | 60 | 18.416 | 93.052 | 185.445 | 1.00 | 43.58 |
| 471 | CG | PHE | A | 60 | 19.579 | 93.957 | 185.491 | 1.00 | 76.01 |
| 472 | CD1 | PHE | A | 60 | 19.616 | 95.002 | 186.399 | 1.00 | 61.74 |
| 473 | CD2 | PHE | A | 60 | 20.634 | 93.787 | 184.606 | 1.00 | 45.10 |
| 474 | CE1 | PHE | A | 60 | 20.691 | 95.867 | 186.419 | 1.00 | 50.78 |
| 475 | CE2 | PHE | A | 60 | 21.712 | 94.651 | 184.621 | 1.00 | 21.53 |
| 476 | CZ | PHE | A | 60 | 21.741 | 95.689 | 185.525 | 1.00 | 37.49 |
| 477 | C | PHE | A | 60 | 16.169 | 92.580 | 184.653 | 1.00 | 69.55 |
| 478 | O | PHE | A | 60 | 16.062 | 91.924 | 183.617 | 1.00 | 29.04 |
| 479 | N | GLU | A | 61 | 15.429 | 92.395 | 185.742 | 1.00 | 35.52 |
| 480 | CA | GLU | A | 61 | 14.371 | 91.406 | 185.823 | 1.00 | 48.73 |
| 481 | CB | GLU | A | 61 | 13.552 | 91.627 | 187.104 | 1.00 | 104.40 |
| 482 | CG | GLU | A | 61 | 14.214 | 91.078 | 188.378 | 1.00 | 176.16 |
| 483 | CD | GLU | A | 61 | 15.427 | 91.882 | 188.853 | 1.00 | 198.10 |
| 484 | OE1 | GLU | A | 61 | 15.236 | 93.016 | 189.341 | 1.00 | 190.74 |
| 485 | OE2 | GLU | A | 61 | 16.573 | 91.381 | 188.747 | 1.00 | 185.84 |
| 486 | C | GLU | A | 61 | 13.468 | 91.481 | 184.602 | 1.00 | 72.09 |
| 487 | O | GLU | A | 61 | 12.846 | 90.496 | 184.214 | 1.00 | 44.44 |
| 488 | N | ASP | A | 62 | 13.418 | 92.657 | 183.987 | 1.00 | 43.54 |
| 489 | CA | ASP | A | 62 | 12.589 | 92.874 | 182.816 | 1.00 | 19.32 |
| 490 | CB | ASP | A | 62 | 12.312 | 94.371 | 182.659 | 1.00 | 5.54 |
| 491 | CG | ASP | A | 62 | 11.524 | 94.956 | 183.850 | 1.00 | 95.59 |
| 492 | OD1 | ASP | A | 62 | 10.790 | 94.200 | 184.540 | 1.00 | 64.35 |
| 493 | OD2 | ASP | A | 62 | 11.618 | 96.182 | 184.091 | 1.00 | 64.15 |
| 494 | C | ASP | A | 62 | 13.156 | 92.283 | 181.519 | 1.00 | 28.80 |
| 495 | O | ASP | A | 62 | 12.492 | 92.276 | 180.483 | 1.00 | 23.08 |
| 496 | N | SER | A | 63 | 14.378 | 91.772 | 181.558 | 1.00 | 39.71 |
| 497 | CA | SER | A | 63 | 14.940 | 91.174 | 180.353 | 1.00 | 51.88 |
| 498 | CB | SER | A | 63 | 16.395 | 90.770 | 180.579 | 1.00 | 43.74 |
| 499 | OG | SER | A | 63 | 17.214 | 91.898 | 180.813 | 1.00 | 80.04 |
| 500 | C | SER | A | 63 | 14.108 | 89.938 | 180.092 | 1.00 | 33.07 |
| 501 | O | SER | A | 63 | 13.290 | 89.573 | 180.919 | 1.00 | 42.64 |
| 502 | N | GLY | A | 64 | 14.295 | 89.293 | 178.949 | 1.00 | 33.31 |
| 503 | CA | GLY | A | 64 | 13.529 | 88.085 | 178.701 | 1.00 | 47.82 |
| 504 | C | GLY | A | 64 | 12.703 | 87.912 | 177.437 | 1.00 | 78.77 |
| 505 | O | GLY | A | 64 | 12.738 | 88.718 | 176.503 | 1.00 | 41.30 |
| 506 | N | GLU | A | 65 | 11.947 | 86.819 | 177.426 | 1.00 | 38.56 |
| 507 | CA | GLU | A | 65 | 11.096 | 86.471 | 176.303 | 1.00 | 61.82 |
| 508 | CB | GLU | A | 65 | 11.022 | 84.950 | 176.157 | 1.00 | 56.45 |
| 509 | CG | GLU | A | 65 | 9.864 | 84.452 | 175.314 | 1.00 | 60.09 |
| 510 | CD | GLU | A | 65 | 9.860 | 82.944 | 175.145 | 1.00 | 97.74 |
| 511 | OE1 | GLU | A | 65 | 9.816 | 82.221 | 176.165 | 1.00 | 145.16 |
| 512 | OE2 | GLU | A | 65 | 9.898 | 82.480 | 173.987 | 1.00 | 99.49 |
| 513 | C | GLU | A | 65 | 9.693 | 87.030 | 176.447 | 1.00 | 47.26 |
| 514 | O | GLU | A | 65 | 9.000 | 86.742 | 177.415 | 1.00 | 50.30 |
| 515 | N | TYR | A | 66 | 9.282 | 87.829 | 175.468 | 1.00 | 55.07 |
| 516 | CA | TYR | A | 66 | 7.951 | 88.414 | 175.462 | 1.00 | 29.51 |
| 517 | CB | TYR | A | 66 | 8.037 | 89.931 | 175.342 | 1.00 | 33.26 |
| 518 | CG | TYR | A | 66 | 8.495 | 90.627 | 176.599 | 1.00 | 36.26 |
| 519 | CD1 | TYR | A | 66 | 9.844 | 90.671 | 176.946 | 1.00 | 54.70 |
| 520 | CE1 | TYR | A | 66 | 10.264 | 91.287 | 178.118 | 1.00 | 20.05 |
| 521 | CD2 | TYR | A | 66 | 7.574 | 91.217 | 177.456 | 1.00 | 14.39 |
| 522 | CE2 | TYR | A | 66 | 7.978 | 91.827 | 178.623 | 1.00 | 52.79 |
| 523 | CZ | TYR | A | 66 | 9.323 | 91.862 | 178.952 | 1.00 | 76.87 |
| 524 | OH | TYR | A | 66 | 9.709 | 92.485 | 180.115 | 1.00 | 49.97 |
| 525 | C | TYR | A | 66 | 7.135 | 87.859 | 174.296 | 1.00 | 58.69 |
| 526 | O | TYR | A | 66 | 7.653 | 87.704 | 173.190 | 1.00 | 49.90 |
| 527 | N | LYS | A | 67 | 5.866 | 87.548 | 174.560 | 1.00 | 53.66 |
| 528 | CA | LYS | A | 67 | 4.946 | 87.023 | 173.550 | 1.00 | 38.25 |
| 529 | CB | LYS | A | 67 | 4.957 | 85.504 | 173.534 | 1.00 | 5.42 |
| 530 | CG | LYS | A | 67 | 6.054 | 84.856 | 172.724 | 1.00 | 46.81 |
| 531 | CD | LYS | A | 67 | 5.918 | 83.324 | 172.794 | 1.00 | 100.90 |
| 532 | CE | LYS | A | 67 | 5.849 | 82.829 | 174.249 | 1.00 | 91.92 |
| 533 | NZ | LYS | A | 67 | 5.762 | 81.347 | 174.372 | 1.00 | 45.56 |
| 534 | C | LYS | A | 67 | 3.530 | 87.460 | 173.867 | 1.00 | 67.55 |
| 535 | O | LYS | A | 67 | 3.164 | 87.560 | 175.038 | 1.00 | 52.62 |
| 536 | N | CYS | A | 68 | 2.741 | 87.730 | 172.830 | 1.00 | 27.93 |
| 537 | CA | CYS | A | 68 | 1.346 | 88.113 | 173.023 | 1.00 | 62.04 |
| 538 | C | CYS | A | 68 | 0.522 | 87.234 | 172.111 | 1.00 | 74.23 |
| 539 | O | CYS | A | 68 | 0.992 | 86.816 | 171.060 | 1.00 | 45.57 |
| 540 | CB | CYS | A | 68 | 1.092 | 89.597 | 172.678 | 1.00 | 29.72 |
| 541 | SG | CYS | A | 68 | 1.419 | 90.098 | 170.952 | 1.00 | 99.54 |
| 542 | N | GLN | A | 69 | −0.695 | 86.920 | 172.525 | 1.00 | 33.27 |
| 543 | CA | GLN | A | 69 | −1.553 | 86.119 | 171.681 | 1.00 | 50.08 |
| 544 | CB | GLN | A | 69 | −1.489 | 84.636 | 172.081 | 1.00 | 48.05 |
| 545 | CG | GLN | A | 69 | −2.425 | 84.215 | 173.203 | 1.00 | 61.10 |
| 546 | CD | GLN | A | 69 | −2.526 | 82.705 | 173.318 | 1.00 | 71.23 |
| 547 | OE1 | GLN | A | 69 | −2.813 | 82.023 | 172.336 | 1.00 | 91.00 |
| 548 | NE2 | GLN | A | 69 | −2.294 | 82.174 | 174.516 | 1.00 | 82.52 |
| 549 | C | GLN | A | 69 | −2.951 | 86.698 | 171.837 | 1.00 | 82.00 |
| 550 | O | GLN | A | 69 | −3.259 | 87.275 | 172.881 | 1.00 | 33.84 |
| 551 | N | HIS | A | 70 | −3.780 | 86.569 | 170.798 | 1.00 | 41.19 |
| 552 | CA | HIS | A | 70 | −5.135 | 87.114 | 170.836 | 1.00 | 43.14 |
| 553 | CB | HIS | A | 70 | −5.503 | 87.695 | 169.484 | 1.00 | 74.38 |
| 554 | CG | HIS | A | 70 | −4.758 | 88.949 | 169.156 | 1.00 | 88.05 |
| 555 | CD2 | HIS | A | 70 | −4.093 | 89.321 | 168.038 | 1.00 | 38.05 |
| 556 | ND1 | HIS | A | 70 | −4.693 | 90.022 | 170.019 | 1.00 | 28.06 |
| 557 | CE1 | HIS | A | 70 | −4.025 | 91.004 | 169.442 | 1.00 | 56.78 |
| 558 | NE2 | HIS | A | 70 | −3.651 | 90.603 | 168.240 | 1.00 | 58.51 |
| 559 | C | HIS | A | 70 | −6.193 | 86.125 | 171.279 | 1.00 | 67.91 |
| 560 | O | HIS | A | 70 | −6.224 | 85.738 | 172.448 | 1.00 | 57.73 |
| 561 | N | GLN | A | 71 | −7.103 | 85.741 | 170.393 | 1.00 | 57.13 |
| 562 | CA | GLN | A | 71 | −8.074 | 84.755 | 170.836 | 1.00 | 92.00 |
| 563 | CB | GLN | A | 71 | −9.521 | 85.291 | 170.854 | 1.00 | 30.07 |
| 564 | CG | GLN | A | 71 | −9.993 | 86.007 | 169.632 | 1.00 | 41.74 |
| 565 | CD | GLN | A | 71 | −11.225 | 86.849 | 169.901 | 1.00 | 87.95 |
| 566 | OE1 | GLN | A | 71 | −11.837 | 86.748 | 170.965 | 1.00 | 35.65 |
| 567 | NE2 | GLN | A | 71 | −11.597 | 87.690 | 168.927 | 1.00 | 53.82 |
| 568 | C | GLN | A | 71 | −7.944 | 83.483 | 170.037 | 1.00 | 34.54 |
| 569 | O | GLN | A | 71 | −8.800 | 82.610 | 170.102 | 1.00 | 84.17 |
| 570 | N | GLN | A | 72 | −6.840 | 83.376 | 169.302 | 1.00 | 51.65 |
| 571 | CA | GLN | A | 72 | −6.556 | 82.167 | 168.543 | 1.00 | 53.66 |
| 572 | CB | GLN | A | 72 | −6.029 | 82.497 | 167.153 | 1.00 | 40.22 |
| 573 | CG | GLN | A | 72 | −7.084 | 82.254 | 166.099 | 1.00 | 83.83 |
| 574 | CD | GLN | A | 72 | −6.726 | 82.827 | 164.759 | 1.00 | 29.94 |
| 575 | OE1 | GLN | A | 72 | −5.765 | 82.397 | 164.129 | 1.00 | 114.76 |
| 576 | NE2 | GLN | A | 72 | −7.500 | 83.813 | 164.310 | 1.00 | 98.66 |
| 577 | C | GLN | A | 72 | −5.573 | 81.267 | 169.288 | 1.00 | 43.67 |
| 578 | O | GLN | A | 72 | −5.373 | 81.404 | 170.490 | 1.00 | 58.65 |
| 579 | N | VAL | A | 73 | −4.958 | 80.337 | 168.583 | 1.00 | 56.83 |
| 580 | CA | VAL | A | 73 | −4.054 | 79.418 | 169.252 | 1.00 | 51.40 |
| 581 | CB | VAL | A | 73 | −4.188 | 77.998 | 168.669 | 1.00 | 106.92 |
| 582 | CG1 | VAL | A | 73 | −3.580 | 77.944 | 167.252 | 1.00 | 46.73 |
| 583 | CG2 | VAL | A | 73 | −3.536 | 76.996 | 169.604 | 1.00 | 22.40 |
| 584 | C | VAL | A | 73 | −2.622 | 79.862 | 169.121 | 1.00 | 64.50 |
| 585 | O | VAL | A | 73 | −1.851 | 79.805 | 170.077 | 1.00 | 72.42 |
| 586 | N | ALA | A | 74 | −2.270 | 80.292 | 167.919 | 1.00 | 78.31 |
| 587 | CA | ALA | A | 74 | −0.924 | 80.744 | 167.649 | 1.00 | 78.26 |
| 588 | CB | ALA | A | 74 | −0.740 | 80.981 | 166.166 | 1.00 | 125.54 |
| 589 | C | ALA | A | 74 | −0.649 | 82.019 | 168.418 | 1.00 | 66.03 |
| 590 | O | ALA | A | 74 | −1.553 | 82.817 | 168.681 | 1.00 | 85.24 |
| 591 | N | GLU | A | 75 | 0.616 | 82.194 | 168.768 | 1.00 | 67.79 |
| 592 | CA | GLU | A | 75 | 1.082 | 83.347 | 169.516 | 1.00 | 53.61 |
| 593 | CB | GLU | A | 75 | 1.465 | 82.908 | 170.935 | 1.00 | 18.85 |
| 594 | CG | GLU | A | 75 | 2.031 | 81.485 | 170.991 | 1.00 | 113.19 |
| 595 | CD | GLU | A | 75 | 2.349 | 81.011 | 172.398 | 1.00 | 127.80 |
| 596 | OE1 | GLU | A | 75 | 1.510 | 81.209 | 173.305 | 1.00 | 114.51 |
| 597 | OE2 | GLU | A | 75 | 3.435 | 80.426 | 172.593 | 1.00 | 173.92 |
| 598 | C | GLU | A | 75 | 2.274 | 83.945 | 168.773 | 1.00 | 48.86 |
| 599 | O | GLU | A | 75 | 3.007 | 83.243 | 168.075 | 1.00 | 57.67 |
| 600 | N | SER | A | 76 | 2.445 | 85.251 | 168.916 | 1.00 | 46.16 |
| 601 | CA | SER | A | 76 | 3.520 | 85.981 | 168.260 | 1.00 | 64.43 |
| 602 | CB | SER | A | 76 | 3.619 | 87.383 | 168.864 | 1.00 | 108.46 |
| 603 | OG | SER | A | 76 | 3.634 | 87.336 | 170.287 | 1.00 | 78.81 |
| 604 | C | SER | A | 76 | 4.865 | 85.291 | 168.375 | 1.00 | 57.59 |
| 605 | O | SER | A | 76 | 5.108 | 84.573 | 169.339 | 1.00 | 53.17 |
| 606 | N | GLU | A | 77 | 5.728 | 85.486 | 167.379 | 1.00 | 95.85 |
| 607 | CA | GLU | A | 77 | 7.064 | 84.906 | 167.442 | 1.00 | 46.97 |
| 608 | CB | GLU | A | 77 | 7.893 | 85.273 | 166.211 | 1.00 | 62.29 |
| 609 | CG | GLU | A | 77 | 7.364 | 84.714 | 164.896 | 1.00 | 130.97 |
| 610 | CD | GLU | A | 77 | 7.571 | 83.215 | 164.760 | 1.00 | 165.99 |
| 611 | OE1 | GLU | A | 77 | 8.743 | 82.773 | 164.758 | 1.00 | 180.77 |
| 612 | OE2 | GLU | A | 77 | 6.566 | 82.477 | 164.650 | 1.00 | 160.47 |
| 613 | C | GLU | A | 77 | 7.579 | 85.645 | 168.662 | 1.00 | 80.10 |
| 614 | O | GLU | A | 77 | 7.229 | 86.804 | 168.880 | 1.00 | 80.13 |
| 615 | N | PRO | A | 78 | 8.410 | 84.999 | 169.476 | 1.00 | 60.47 |
| 616 | CD | PRO | A | 78 | 9.153 | 83.748 | 169.255 | 1.00 | 68.85 |
| 617 | CA | PRO | A | 78 | 8.902 | 85.692 | 170.661 | 1.00 | 58.42 |
| 618 | CB | PRO | A | 78 | 9.606 | 84.583 | 171.413 | 1.00 | 55.27 |
| 619 | CG | PRO | A | 78 | 10.270 | 83.853 | 170.283 | 1.00 | 88.80 |
| 620 | C | PRO | A | 78 | 9.836 | 86.843 | 170.322 | 1.00 | 68.90 |
| 621 | O | PRO | A | 78 | 10.461 | 86.855 | 169.263 | 1.00 | 56.81 |
| 622 | N | VAL | A | 79 | 9.914 | 87.809 | 171.231 | 1.00 | 55.88 |
| 623 | CA | VAL | A | 79 | 10.785 | 88.963 | 171.075 | 1.00 | 53.98 |
| 624 | CB | VAL | A | 79 | 9.980 | 90.251 | 170.992 | 1.00 | 34.06 |
| 625 | CG1 | VAL | A | 79 | 10.910 | 91.426 | 170.825 | 1.00 | 85.73 |
| 626 | CG2 | VAL | A | 79 | 9.028 | 90.171 | 169.833 | 1.00 | 97.73 |
| 627 | C | VAL | A | 79 | 11.649 | 89.009 | 172.321 | 1.00 | 87.70 |
| 628 | O | VAL | A | 79 | 11.119 | 89.113 | 173.433 | 1.00 | 48.94 |
| 629 | N | TYR | A | 80 | 12.968 | 88.928 | 172.142 | 1.00 | 53.61 |
| 630 | CA | TYR | A | 80 | 13.878 | 88.944 | 173.278 | 1.00 | 34.81 |
| 631 | CB | TYR | A | 80 | 15.023 | 87.972 | 173.020 | 1.00 | 65.29 |
| 632 | CG | TYR | A | 80 | 14.516 | 86.557 | 172.856 | 1.00 | 94.10 |
| 633 | CD1 | TYR | A | 80 | 14.499 | 85.938 | 171.605 | 1.00 | 67.48 |
| 634 | CE1 | TYR | A | 80 | 13.965 | 84.657 | 171.442 | 1.00 | 77.57 |
| 635 | CD2 | TYR | A | 80 | 13.989 | 85.856 | 173.947 | 1.00 | 68.38 |
| 636 | CE2 | TYR | A | 80 | 13.451 | 84.578 | 173.797 | 1.00 | 65.53 |
| 637 | CZ | TYR | A | 80 | 13.440 | 83.985 | 172.542 | 1.00 | 93.69 |
| 638 | OH | TYR | A | 80 | 12.885 | 82.733 | 172.385 | 1.00 | 86.31 |
| 639 | C | TYR | A | 80 | 14.390 | 90.337 | 173.627 | 1.00 | 56.78 |
| 640 | O | TYR | A | 80 | 14.892 | 91.069 | 172.777 | 1.00 | 63.63 |
| 641 | N | LEU | A | 81 | 14.244 | 90.689 | 174.899 | 1.00 | 10.90 |
| 642 | CA | LEU | A | 81 | 14.628 | 91.993 | 175.411 | 1.00 | 19.84 |
| 643 | CB | LEU | A | 81 | 13.412 | 92.635 | 176.068 | 1.00 | 16.84 |
| 644 | CG | LEU | A | 81 | 13.534 | 94.104 | 176.437 | 1.00 | 35.65 |
| 645 | CD1 | LEU | A | 81 | 13.214 | 94.911 | 175.209 | 1.00 | 77.33 |
| 646 | CD2 | LEU | A | 81 | 12.585 | 94.468 | 177.544 | 1.00 | 33.43 |
| 647 | C | LEU | A | 81 | 15.748 | 91.903 | 176.441 | 1.00 | 46.78 |
| 648 | O | LEU | A | 81 | 15.522 | 91.419 | 177.549 | 1.00 | 42.71 |
| 649 | N | GLU | A | 82 | 16.939 | 92.393 | 176.098 | 1.00 | 20.80 |
| 650 | CA | GLU | A | 82 | 18.070 | 92.348 | 177.024 | 1.00 | 45.93 |
| 651 | CB | GLU | A | 82 | 19.318 | 91.828 | 176.307 | 1.00 | 24.73 |
| 652 | CG | GLU | A | 82 | 19.115 | 90.473 | 175.638 | 1.00 | 154.76 |
| 653 | CD | GLU | A | 82 | 20.294 | 90.041 | 174.785 | 1.00 | 173.29 |
| 654 | OE1 | GLU | A | 82 | 21.395 | 89.842 | 175.353 | 1.00 | 124.38 |
| 655 | OE2 | GLU | A | 82 | 20.112 | 89.903 | 173.550 | 1.00 | 145.47 |
| 656 | C | GLU | A | 82 | 18.361 | 93.713 | 177.634 | 1.00 | 51.76 |
| 657 | O | GLU | A | 82 | 18.443 | 94.705 | 176.919 | 1.00 | 33.44 |
| 658 | N | VAL | A | 83 | 18.514 | 93.756 | 178.957 | 1.00 | 5.42 |
| 659 | CA | VAL | A | 83 | 18.800 | 95.000 | 179.663 | 1.00 | 45.28 |
| 660 | CB | VAL | A | 83 | 17.904 | 95.144 | 180.900 | 1.00 | 55.93 |
| 661 | CG1 | VAL | A | 83 | 18.181 | 96.457 | 181.587 | 1.00 | 84.34 |
| 662 | CG2 | VAL | A | 83 | 16.457 | 95.065 | 180.499 | 1.00 | 47.82 |
| 663 | C | VAL | A | 83 | 20.263 | 95.031 | 180.108 | 1.00 | 52.84 |
| 664 | O | VAL | A | 83 | 20.694 | 94.195 | 180.901 | 1.00 | 69.13 |
| 665 | N | PHE | A | 84 | 21.018 | 96.007 | 179.610 | 1.00 | 62.21 |
| 666 | CA | PHE | A | 84 | 22.439 | 96.126 | 179.926 | 1.00 | 41.74 |
| 667 | CB | PHE | A | 84 | 23.249 | 96.308 | 178.653 | 1.00 | 33.99 |
| 668 | CG | PHE | A | 84 | 23.117 | 95.196 | 177.675 | 1.00 | 27.72 |
| 669 | CD1 | PHE | A | 84 | 21.904 | 94.910 | 177.096 | 1.00 | 35.75 |
| 670 | CD2 | PHE | A | 84 | 24.230 | 94.459 | 177.298 | 1.00 | 63.18 |
| 671 | CE1 | PHE | A | 84 | 21.797 | 93.908 | 176.149 | 1.00 | 106.48 |
| 672 | CE2 | PHE | A | 84 | 24.135 | 93.457 | 176.353 | 1.00 | 72.91 |
| 673 | CZ | PHE | A | 84 | 22.917 | 93.179 | 175.776 | 1.00 | 70.31 |
| 674 | C | PHE | A | 84 | 22.807 | 97.288 | 180.825 | 1.00 | 50.31 |
| 675 | O | PHE | A | 84 | 21.989 | 98.152 | 181.116 | 1.00 | 51.85 |
| 676 | N | SER | A | 85 | 24.075 | 97.293 | 181.228 | 1.00 | 58.67 |
| 677 | CA | SER | A | 85 | 24.676 | 98.342 | 182.054 | 1.00 | 62.35 |
| 678 | CB | SER | A | 85 | 24.515 | 98.072 | 183.537 | 1.00 | 63.27 |
| 679 | OG | SER | A | 85 | 25.416 | 98.903 | 184.249 | 1.00 | 42.99 |
| 680 | C | SER | A | 85 | 26.162 | 98.412 | 181.760 | 1.00 | 20.41 |
| 681 | O | SER | A | 85 | 26.969 | 97.836 | 182.480 | 1.00 | 71.62 |
| 682 | N | ASP | A | 86 | 26.503 | 99.124 | 180.694 | 1.00 | 57.01 |
| 683 | CA | ASP | A | 86 | 27.873 | 99.290 | 180.252 | 1.00 | 18.49 |
| 684 | CB | ASP | A | 86 | 28.179 | 98.274 | 179.158 | 1.00 | 54.07 |
| 685 | CG | ASP | A | 86 | 29.651 | 98.139 | 178.883 | 1.00 | 94.47 |
| 686 | OD1 | ASP | A | 86 | 30.251 | 99.061 | 178.284 | 1.00 | 107.01 |
| 687 | OD2 | ASP | A | 86 | 30.210 | 97.095 | 179.277 | 1.00 | 121.47 |
| 688 | C | ASP | A | 86 | 28.036 | 100.719 | 179.739 | 1.00 | 38.14 |
| 689 | O | ASP | A | 86 | 27.162 | 101.559 | 179.934 | 1.00 | 28.17 |
| 690 | N | TRP | A | 87 | 29.143 | 101.007 | 179.080 | 1.00 | 27.21 |
| 691 | CA | TRP | A | 87 | 29.360 | 102.359 | 178.621 | 1.00 | 5.42 |
| 692 | CB | TRP | A | 87 | 30.850 | 102.677 | 178.615 | 1.00 | 66.21 |
| 693 | CG | TRP | A | 87 | 31.410 | 102.892 | 179.971 | 1.00 | 5.42 |
| 694 | CD2 | TRP | A | 87 | 31.589 | 104.143 | 180.619 | 1.00 | 22.62 |
| 695 | CE2 | TRP | A | 87 | 32.119 | 103.885 | 181.897 | 1.00 | 32.39 |
| 696 | CE3 | TRP | A | 87 | 31.356 | 105.467 | 180.243 | 1.00 | 27.34 |
| 697 | CD1 | TRP | A | 87 | 31.831 | 101.934 | 180.863 | 1.00 | 80.10 |
| 698 | NE1 | TRP | A | 87 | 32.259 | 102.528 | 182.026 | 1.00 | 27.00 |
| 699 | CZ2 | TRP | A | 87 | 32.415 | 104.901 | 182.795 | 1.00 | 66.07 |
| 700 | CZ3 | TRP | A | 87 | 31.650 | 106.465 | 181.127 | 1.00 | 5.42 |
| 701 | CH2 | TRP | A | 87 | 32.174 | 106.185 | 182.389 | 1.00 | 51.52 |
| 702 | C | TRP | A | 87 | 28.756 | 102.597 | 177.260 | 1.00 | 55.21 |
| 703 | O | TRP | A | 87 | 28.120 | 103.633 | 177.043 | 1.00 | 35.93 |
| 704 | N | LEU | A | 88 | 28.962 | 101.657 | 176.340 | 1.00 | 11.34 |
| 705 | CA | LEU | A | 88 | 28.380 | 101.771 | 175.005 | 1.00 | 32.20 |
| 706 | CB | LEU | A | 88 | 29.448 | 101.985 | 173.934 | 1.00 | 5.42 |
| 707 | CG | LEU | A | 88 | 30.149 | 103.334 | 174.042 | 1.00 | 25.97 |
| 708 | CD1 | LEU | A | 88 | 30.908 | 103.647 | 172.772 | 1.00 | 8.11 |
| 709 | CD2 | LEU | A | 88 | 29.107 | 104.407 | 174.305 | 1.00 | 18.42 |
| 710 | C | LEU | A | 88 | 27.579 | 100.530 | 174.686 | 1.00 | 40.80 |
| 711 | O | LEU | A | 88 | 27.996 | 99.411 | 174.969 | 1.00 | 71.11 |
| 712 | N | LEU | A | 89 | 26.413 | 100.743 | 174.097 | 1.00 | 29.92 |
| 713 | CA | LEU | A | 89 | 25.514 | 99.660 | 173.727 | 1.00 | 40.51 |
| 714 | CB | LEU | A | 89 | 24.232 | 99.746 | 174.553 | 1.00 | 29.28 |
| 715 | CG | LEU | A | 89 | 23.183 | 98.661 | 174.363 | 1.00 | 41.41 |
| 716 | CD1 | LEU | A | 89 | 23.364 | 97.607 | 175.406 | 1.00 | 8.36 |
| 717 | CD2 | LEU | A | 89 | 21.807 | 99.251 | 174.519 | 1.00 | 110.85 |
| 718 | C | LEU | A | 89 | 25.186 | 99.855 | 172.258 | 1.00 | 55.63 |
| 719 | O | LEU | A | 89 | 24.869 | 100.964 | 171.825 | 1.00 | 37.39 |
| 720 | N | LEU | A | 90 | 25.293 | 98.792 | 171.477 | 1.00 | 38.50 |
| 721 | CA | LEU | A | 90 | 24.983 | 98.911 | 170.063 | 1.00 | 17.59 |
| 722 | CB | LEU | A | 90 | 25.917 | 98.066 | 169.229 | 1.00 | 8.86 |
| 723 | CG | LEU | A | 90 | 25.566 | 98.059 | 167.755 | 1.00 | 23.44 |
| 724 | CD1 | LEU | A | 90 | 26.146 | 99.279 | 167.081 | 1.00 | 27.66 |
| 725 | CD2 | LEU | A | 90 | 26.117 | 96.794 | 167.137 | 1.00 | 25.45 |
| 726 | C | LEU | A | 90 | 23.587 | 98.383 | 169.924 | 1.00 | 38.66 |
| 727 | O | LEU | A | 90 | 23.330 | 97.213 | 170.195 | 1.00 | 48.96 |
| 728 | N | GLN | A | 91 | 22.681 | 99.251 | 169.505 | 1.00 | 36.75 |
| 729 | CA | GLN | A | 91 | 21.295 | 98.871 | 169.354 | 1.00 | 8.29 |
| 730 | CB | GLN | A | 91 | 20.401 | 99.929 | 169.966 | 1.00 | 39.59 |
| 731 | CG | GLN | A | 91 | 20.488 | 100.046 | 171.453 | 1.00 | 5.42 |
| 732 | CD | GLN | A | 91 | 19.685 | 101.217 | 171.942 | 1.00 | 54.77 |
| 733 | OE1 | GLN | A | 91 | 19.702 | 102.288 | 171.325 | 1.00 | 27.61 |
| 734 | NE2 | GLN | A | 91 | 18.983 | 101.036 | 173.053 | 1.00 | 32.79 |
| 735 | C | GLN | A | 91 | 20.927 | 98.713 | 167.903 | 1.00 | 57.80 |
| 736 | O | GLN | A | 91 | 21.387 | 99.472 | 167.049 | 1.00 | 61.80 |
| 737 | N | ALA | A | 92 | 20.083 | 97.727 | 167.624 | 1.00 | 41.69 |
| 738 | CA | ALA | A | 92 | 19.652 | 97.509 | 166.262 | 1.00 | 33.39 |
| 739 | CB | ALA | A | 92 | 20.287 | 96.263 | 165.698 | 1.00 | 53.81 |
| 740 | C | ALA | A | 92 | 18.147 | 97.400 | 166.213 | 1.00 | 66.17 |
| 741 | O | ALA | A | 92 | 17.518 | 96.880 | 167.141 | 1.00 | 43.51 |
| 742 | N | SER | A | 93 | 17.592 | 97.919 | 165.121 | 1.00 | 31.27 |
| 743 | CA | SER | A | 93 | 16.163 | 97.913 | 164.863 | 1.00 | 58.56 |
| 744 | CB | SER | A | 93 | 15.890 | 98.616 | 163.541 | 1.00 | 37.57 |
| 745 | OG | SER | A | 93 | 16.522 | 97.916 | 162.484 | 1.00 | 75.71 |
| 746 | C | SER | A | 93 | 15.629 | 96.478 | 164.810 | 1.00 | 81.43 |
| 747 | O | SER | A | 93 | 14.493 | 96.216 | 165.208 | 1.00 | 48.00 |
| 748 | N | ALA | A | 94 | 16.454 | 95.560 | 164.317 | 1.00 | 14.94 |
| 749 | CA | ALA | A | 94 | 16.084 | 94.157 | 164.218 | 1.00 | 54.65 |
| 750 | CB | ALA | A | 94 | 15.137 | 93.958 | 163.058 | 1.00 | 117.54 |
| 751 | C | ALA | A | 94 | 17.323 | 93.302 | 164.028 | 1.00 | 55.96 |
| 752 | O | ALA | A | 94 | 18.162 | 93.613 | 163.198 | 1.00 | 62.00 |
| 753 | N | GLU | A | 95 | 17.433 | 92.214 | 164.780 | 1.00 | 54.57 |
| 754 | CA | GLU | A | 95 | 18.605 | 91.356 | 164.667 | 1.00 | 61.37 |
| 755 | CB | GLU | A | 95 | 18.722 | 90.493 | 165.917 | 1.00 | 82.00 |
| 756 | CG | GLU | A | 95 | 18.730 | 91.321 | 167.184 | 1.00 | 46.60 |
| 757 | CD | GLU | A | 95 | 18.734 | 90.472 | 168.427 | 1.00 | 114.77 |
| 758 | OE1 | GLU | A | 95 | 17.813 | 89.639 | 168.582 | 1.00 | 140.88 |
| 759 | OE2 | GLU | A | 95 | 19.659 | 90.640 | 169.247 | 1.00 | 105.98 |
| 760 | C | GLU | A | 95 | 18.598 | 90.494 | 163.408 | 1.00 | 47.61 |
| 761 | O | GLU | A | 95 | 19.650 | 90.135 | 162.888 | 1.00 | 68.07 |
| 762 | N | VAL | A | 96 | 17.407 | 90.163 | 162.926 | 1.00 | 79.01 |
| 763 | CA | VAL | A | 96 | 17.246 | 89.377 | 161.703 | 1.00 | 53.09 |
| 764 | CB | VAL | A | 96 | 16.572 | 88.026 | 161.974 | 1.00 | 60.34 |
| 765 | CG1 | VAL | A | 96 | 16.439 | 87.269 | 160.687 | 1.00 | 66.62 |
| 766 | CG2 | VAL | A | 96 | 17.384 | 87.219 | 162.972 | 1.00 | 75.27 |
| 767 | C | VAL | A | 96 | 16.348 | 90.214 | 160.801 | 1.00 | 37.17 |
| 768 | O | VAL | A | 96 | 15.182 | 90.453 | 161.107 | 1.00 | 67.51 |
| 769 | N | VAL | A | 97 | 16.900 | 90.673 | 159.692 | 1.00 | 31.53 |
| 770 | CA | VAL | A | 97 | 16.164 | 91.532 | 158.786 | 1.00 | 61.27 |
| 771 | CB | VAL | A | 97 | 16.945 | 92.851 | 158.561 | 1.00 | 37.63 |
| 772 | CG1 | VAL | A | 97 | 16.712 | 93.382 | 157.165 | 1.00 | 101.90 |
| 773 | CG2 | VAL | A | 97 | 16.501 | 93.882 | 159.579 | 1.00 | 109.18 |
| 774 | C | VAL | A | 97 | 15.894 | 90.875 | 157.450 | 1.00 | 74.73 |
| 775 | O | VAL | A | 97 | 16.828 | 90.509 | 156.745 | 1.00 | 91.83 |
| 776 | N | MET | A | 98 | 14.615 | 90.734 | 157.103 | 1.00 | 107.16 |
| 777 | CA | MET | A | 98 | 14.229 | 90.126 | 155.835 | 1.00 | 85.06 |
| 778 | CB | MET | A | 98 | 12.701 | 90.028 | 155.717 | 1.00 | 152.68 |
| 779 | CG | MET | A | 98 | 12.042 | 89.134 | 156.758 | 1.00 | 194.20 |
| 780 | SD | MET | A | 98 | 10.239 | 89.149 | 156.658 | 1.00 | 216.44 |
| 781 | CE | MET | A | 98 | 9.857 | 90.576 | 157.704 | 1.00 | 214.75 |
| 782 | C | MET | A | 98 | 14.778 | 90.978 | 154.699 | 1.00 | 101.32 |
| 783 | O | MET | A | 98 | 14.724 | 92.203 | 154.747 | 1.00 | 54.34 |
| 784 | N | GLU | A | 99 | 15.315 | 90.312 | 153.684 | 1.00 | 129.05 |
| 785 | CA | GLU | A | 99 | 15.895 | 90.956 | 152.506 | 1.00 | 108.86 |
| 786 | CB | GLU | A | 99 | 15.975 | 89.918 | 151.373 | 1.00 | 189.93 |
| 787 | CG | GLU | A | 99 | 16.759 | 90.309 | 150.123 | 1.00 | 203.35 |
| 788 | CD | GLU | A | 99 | 16.928 | 89.132 | 149.164 | 1.00 | 206.77 |
| 789 | OE1 | GLU | A | 99 | 15.908 | 88.496 | 148.810 | 1.00 | 194.67 |
| 790 | OE2 | GLU | A | 99 | 18.079 | 88.844 | 148.765 | 1.00 | 186.48 |
| 791 | C | GLU | A | 99 | 15.083 | 92.177 | 152.061 | 1.00 | 72.06 |
| 792 | O | GLU | A | 99 | 13.856 | 92.132 | 152.005 | 1.00 | 110.93 |
| 793 | N | GLY | A | 100 | 15.770 | 93.271 | 151.758 | 1.00 | 62.36 |
| 794 | CA | GLY | A | 100 | 15.080 | 94.470 | 151.312 | 1.00 | 77.49 |
| 795 | C | GLY | A | 100 | 14.648 | 95.464 | 152.380 | 1.00 | 106.99 |
| 796 | O | GLY | A | 100 | 14.773 | 96.675 | 152.180 | 1.00 | 142.90 |
| 797 | N | GLN | A | 101 | 14.134 | 94.967 | 153.505 | 1.00 | 76.10 |
| 798 | CA | GLN | A | 101 | 13.691 | 95.830 | 154.606 | 1.00 | 99.54 |
| 799 | CB | GLN | A | 101 | 13.125 | 94.970 | 155.749 | 1.00 | 118.17 |
| 800 | CG | GLN | A | 101 | 11.942 | 94.075 | 155.371 | 1.00 | 127.37 |
| 801 | CD | GLN | A | 101 | 10.670 | 94.847 | 155.045 | 1.00 | 143.15 |
| 802 | OE1 | GLN | A | 101 | 10.628 | 96.072 | 155.148 | 1.00 | 128.68 |
| 803 | NE2 | GLN | A | 101 | 9.622 | 94.125 | 154.657 | 1.00 | 171.66 |
| 804 | C | GLN | A | 101 | 14.821 | 96.737 | 155.149 | 1.00 | 80.68 |
| 805 | O | GLN | A | 101 | 16.007 | 96.446 | 154.978 | 1.00 | 81.82 |
| 806 | N | PRO | A | 102 | 14.461 | 97.854 | 155.803 | 1.00 | 39.03 |
| 807 | CD | PRO | A | 102 | 13.105 | 98.404 | 155.959 | 1.00 | 80.04 |
| 808 | CA | PRO | A | 102 | 15.446 | 98.779 | 156.359 | 1.00 | 46.08 |
| 809 | CB | PRO | A | 102 | 14.633 | 100.039 | 156.586 | 1.00 | 68.48 |
| 810 | CG | PRO | A | 102 | 13.316 | 99.493 | 156.982 | 1.00 | 39.46 |
| 811 | C | PRO | A | 102 | 16.106 | 98.283 | 157.645 | 1.00 | 68.21 |
| 812 | O | PRO | A | 102 | 15.560 | 97.432 | 158.365 | 1.00 | 33.76 |
| 813 | N | LEU | A | 103 | 17.280 | 98.847 | 157.924 | 1.00 | 47.86 |
| 814 | CA | LEU | A | 103 | 18.070 | 98.496 | 159.093 | 1.00 | 42.26 |
| 815 | CB | LEU | A | 103 | 19.201 | 97.587 | 158.673 | 1.00 | 5.42 |
| 816 | CG | LEU | A | 103 | 20.077 | 97.146 | 159.825 | 1.00 | 53.71 |
| 817 | CD1 | LEU | A | 103 | 19.245 | 96.340 | 160.809 | 1.00 | 30.09 |
| 818 | CD2 | LEU | A | 103 | 21.239 | 96.342 | 159.271 | 1.00 | 40.52 |
| 819 | C | LEU | A | 103 | 18.656 | 99.713 | 159.791 | 1.00 | 39.88 |
| 820 | O | LEU | A | 103 | 19.091 | 100.667 | 159.149 | 1.00 | 48.87 |
| 821 | N | PHE | A | 104 | 18.690 | 99.675 | 161.113 | 1.00 | 16.90 |
| 822 | CA | PHE | A | 104 | 19.229 | 100.796 | 161.853 | 1.00 | 47.53 |
| 823 | CB | PHE | A | 104 | 18.107 | 101.655 | 162.406 | 1.00 | 17.73 |
| 824 | CG | PHE | A | 104 | 17.195 | 102.171 | 161.361 | 1.00 | 55.90 |
| 825 | CD1 | PHE | A | 104 | 15.965 | 101.566 | 161.139 | 1.00 | 15.89 |
| 826 | CD2 | PHE | A | 104 | 17.569 | 103.257 | 160.577 | 1.00 | 76.37 |
| 827 | CE1 | PHE | A | 104 | 15.105 | 102.040 | 160.146 | 1.00 | 68.24 |
| 828 | CE2 | PHE | A | 104 | 16.719 | 103.743 | 159.579 | 1.00 | 94.38 |
| 829 | CZ | PHE | A | 104 | 15.481 | 103.133 | 159.363 | 1.00 | 50.60 |
| 830 | C | PHE | A | 104 | 20.150 | 100.419 | 162.978 | 1.00 | 42.37 |
| 831 | O | PHE | A | 104 | 19.834 | 99.593 | 163.832 | 1.00 | 47.93 |
| 832 | N | LEU | A | 105 | 21.306 | 101.049 | 162.970 | 1.00 | 26.80 |
| 833 | CA | LEU | A | 105 | 22.277 | 100.811 | 163.995 | 1.00 | 33.05 |
| 834 | CB | LEU | A | 105 | 23.609 | 100.437 | 163.362 | 1.00 | 47.90 |
| 835 | CG | LEU | A | 105 | 23.596 | 99.200 | 162.464 | 1.00 | 47.60 |
| 836 | CD1 | LEU | A | 105 | 24.941 | 99.044 | 161.777 | 1.00 | 83.01 |
| 837 | CD2 | LEU | A | 105 | 23.285 | 97.976 | 163.291 | 1.00 | 13.75 |
| 838 | C | LEU | A | 105 | 22.391 | 102.129 | 164.720 | 1.00 | 52.31 |
| 839 | O | LEU | A | 105 | 22.230 | 103.192 | 164.123 | 1.00 | 53.94 |
| 840 | N | ARG | A | 106 | 22.676 | 102.055 | 166.010 | 1.00 | 21.83 |
| 841 | CA | ARG | A | 106 | 22.819 | 103.239 | 166.837 | 1.00 | 41.63 |
| 842 | CB | ARG | A | 106 | 21.456 | 103.568 | 167.438 | 1.00 | 23.32 |
| 843 | CG | ARG | A | 106 | 21.448 | 104.739 | 168.369 | 1.00 | 51.86 |
| 844 | CD | ARG | A | 106 | 20.098 | 104.857 | 169.030 | 1.00 | 30.81 |
| 845 | NE | ARG | A | 106 | 20.162 | 105.533 | 170.318 | 1.00 | 31.36 |
| 846 | CZ | ARG | A | 106 | 19.100 | 105.803 | 171.065 | 1.00 | 52.13 |
| 847 | NH1 | ARG | A | 106 | 17.892 | 105.459 | 170.642 | 1.00 | 93.33 |
| 848 | NH2 | ARG | A | 106 | 19.240 | 106.401 | 172.241 | 1.00 | 34.31 |
| 849 | C | ARG | A | 106 | 23.838 | 102.977 | 167.956 | 1.00 | 57.42 |
| 850 | O | ARG | A | 106 | 23.716 | 101.991 | 168.685 | 1.00 | 30.30 |
| 851 | N | CYS | A | 107 | 24.866 | 103.815 | 168.077 | 1.00 | 24.37 |
| 852 | CA | CYS | A | 107 | 25.807 | 103.616 | 169.176 | 1.00 | 42.73 |
| 853 | C | CYS | A | 107 | 25.176 | 104.464 | 170.251 | 1.00 | 44.66 |
| 854 | O | CYS | A | 107 | 25.065 | 105.682 | 170.102 | 1.00 | 52.70 |
| 855 | CB | CYS | A | 107 | 27.216 | 104.119 | 168.858 | 1.00 | 5.42 |
| 856 | SG | CYS | A | 107 | 28.525 | 103.331 | 169.872 | 1.00 | 82.53 |
| 857 | N | HIS | A | 108 | 24.717 | 103.804 | 171.311 | 1.00 | 11.14 |
| 858 | CA | HIS | A | 108 | 24.045 | 104.487 | 172.398 | 1.00 | 21.95 |
| 859 | CB | HIS | A | 108 | 22.770 | 103.755 | 172.765 | 1.00 | 13.74 |
| 860 | CG | HIS | A | 108 | 21.954 | 104.478 | 173.783 | 1.00 | 50.61 |
| 861 | CD2 | HIS | A | 108 | 21.270 | 104.037 | 174.864 | 1.00 | 13.51 |
| 862 | ND1 | HIS | A | 108 | 21.783 | 105.845 | 173.750 | 1.00 | 39.22 |
| 863 | CE1 | HIS | A | 108 | 21.030 | 106.213 | 174.769 | 1.00 | 67.80 |
| 864 | NE2 | HIS | A | 108 | 20.706 | 105.135 | 175.460 | 1.00 | 46.62 |
| 865 | C | HIS | A | 108 | 24.898 | 104.633 | 173.634 | 1.00 | 52.09 |
| 866 | O | HIS | A | 108 | 25.330 | 103.648 | 174.220 | 1.00 | 15.69 |
| 867 | N | GLY | A | 109 | 25.122 | 105.874 | 174.047 | 1.00 | 37.30 |
| 868 | CA | GLY | A | 109 | 25.950 | 106.085 | 175.212 | 1.00 | 36.86 |
| 869 | C | GLY | A | 109 | 25.165 | 106.142 | 176.498 | 1.00 | 46.50 |
| 870 | O | GLY | A | 109 | 24.159 | 106.838 | 176.579 | 1.00 | 42.12 |
| 871 | N | TRP | A | 110 | 25.641 | 105.412 | 177.501 | 1.00 | 7.94 |
| 872 | CA | TRP | A | 110 | 25.033 | 105.365 | 178.824 | 1.00 | 30.83 |
| 873 | CB | TRP | A | 110 | 26.075 | 104.901 | 179.816 | 1.00 | 5.42 |
| 874 | CG | TRP | A | 110 | 25.536 | 104.902 | 181.182 | 1.00 | 67.67 |
| 875 | CD2 | TRP | A | 110 | 24.735 | 103.879 | 181.772 | 1.00 | 56.37 |
| 876 | CE2 | TRP | A | 110 | 24.444 | 104.283 | 183.085 | 1.00 | 18.12 |
| 877 | CE3 | TRP | A | 110 | 24.239 | 102.654 | 181.316 | 1.00 | 81.28 |
| 878 | CD1 | TRP | A | 110 | 25.692 | 105.866 | 182.129 | 1.00 | 37.15 |
| 879 | NE1 | TRP | A | 110 | 25.039 | 105.500 | 183.283 | 1.00 | 90.09 |
| 880 | CZ2 | TRP | A | 110 | 23.685 | 103.510 | 183.944 | 1.00 | 53.89 |
| 881 | CZ3 | TRP | A | 110 | 23.484 | 101.886 | 182.176 | 1.00 | 86.79 |
| 882 | CH2 | TRP | A | 110 | 23.216 | 102.317 | 183.476 | 1.00 | 35.12 |
| 883 | C | TRP | A | 110 | 24.430 | 106.697 | 179.304 | 1.00 | 25.46 |
| 884 | O | TRP | A | 110 | 24.984 | 107.758 | 179.042 | 1.00 | 34.32 |
| 885 | N | ARG | A | 111 | 23.324 | 106.639 | 180.045 | 1.00 | 9.65 |
| 886 | CA | ARG | A | 111 | 22.640 | 107.855 | 180.509 | 1.00 | 64.56 |
| 887 | CB | ARG | A | 111 | 23.299 | 108.434 | 181.778 | 1.00 | 5.57 |
| 888 | CG | ARG | A | 111 | 23.045 | 107.558 | 183.026 | 1.00 | 122.90 |
| 889 | CD | ARG | A | 111 | 23.274 | 108.250 | 184.384 | 1.00 | 92.09 |
| 890 | NE | ARG | A | 111 | 22.026 | 108.704 | 185.001 | 1.00 | 66.00 |
| 891 | CZ | ARG | A | 111 | 21.283 | 109.706 | 184.540 | 1.00 | 111.74 |
| 892 | NH1 | ARG | A | 111 | 21.659 | 110.370 | 183.454 | 1.00 | 85.41 |
| 893 | NH2 | ARG | A | 111 | 20.159 | 110.045 | 185.159 | 1.00 | 106.66 |
| 894 | C | ARG | A | 111 | 22.590 | 108.889 | 179.383 | 1.00 | 25.39 |
| 895 | O | ARG | A | 111 | 22.455 | 110.096 | 179.584 | 1.00 | 35.74 |
| 896 | N | ASN | A | 112 | 22.668 | 108.367 | 178.176 | 1.00 | 22.55 |
| 897 | CA | ASN | A | 112 | 22.638 | 109.160 | 176.979 | 1.00 | 55.36 |
| 898 | CB | ASN | A | 112 | 21.259 | 109.811 | 176.809 | 1.00 | 23.18 |
| 899 | CG | ASN | A | 112 | 20.903 | 110.048 | 175.338 | 1.00 | 99.46 |
| 900 | OD1 | ASN | A | 112 | 21.105 | 109.180 | 174.484 | 1.00 | 76.98 |
| 901 | ND2 | ASN | A | 112 | 20.363 | 111.225 | 175.042 | 1.00 | 120.92 |
| 902 | C | ASN | A | 112 | 23.759 | 110.186 | 177.006 | 1.00 | 30.60 |
| 903 | O | ASN | A | 112 | 23.623 | 111.301 | 176.503 | 1.00 | 78.43 |
| 904 | N | TRP | A | 113 | 24.884 | 109.793 | 177.590 | 1.00 | 28.24 |
| 905 | CA | TRP | A | 113 | 26.034 | 110.674 | 177.632 | 1.00 | 46.75 |
| 906 | CB | TRP | A | 113 | 27.127 | 110.128 | 178.532 | 1.00 | 19.32 |
| 907 | CG | TRP | A | 113 | 26.882 | 110.334 | 179.969 | 1.00 | 11.92 |
| 908 | CD2 | TRP | A | 113 | 27.516 | 109.646 | 181.050 | 1.00 | 26.29 |
| 909 | CE2 | TRP | A | 113 | 27.000 | 110.174 | 182.242 | 1.00 | 5.42 |
| 910 | CE3 | TRP | A | 113 | 28.477 | 108.636 | 181.124 | 1.00 | 22.82 |
| 911 | CD1 | TRP | A | 113 | 26.037 | 111.226 | 180.528 | 1.00 | 35.81 |
| 912 | NE1 | TRP | A | 113 | 26.095 | 111.138 | 181.899 | 1.00 | 39.35 |
| 913 | CZ2 | TRP | A | 113 | 27.411 | 109.724 | 183.501 | 1.00 | 42.09 |
| 914 | CZ3 | TRP | A | 113 | 28.886 | 108.191 | 182.375 | 1.00 | 13.91 |
| 915 | CH2 | TRP | A | 113 | 28.355 | 108.733 | 183.542 | 1.00 | 5.42 |
| 916 | C | TRP | A | 113 | 26.585 | 110.818 | 176.232 | 1.00 | 39.58 |
| 917 | O | TRP | A | 113 | 26.479 | 109.921 | 175.397 | 1.00 | 56.66 |
| 918 | N | ASP | A | 114 | 27.174 | 111.970 | 175.983 | 1.00 | 40.70 |
| 919 | CA | ASP | A | 114 | 27.749 | 112.254 | 174.696 | 1.00 | 44.29 |
| 920 | CB | ASP | A | 114 | 28.223 | 113.698 | 174.678 | 1.00 | 49.96 |
| 921 | CG | ASP | A | 114 | 27.094 | 114.663 | 174.860 | 1.00 | 31.77 |
| 922 | OD1 | ASP | A | 114 | 26.224 | 114.722 | 173.964 | 1.00 | 75.41 |
| 923 | OD2 | ASP | A | 114 | 27.072 | 115.349 | 175.897 | 1.00 | 54.49 |
| 924 | C | ASP | A | 114 | 28.910 | 111.324 | 174.393 | 1.00 | 58.03 |
| 925 | O | ASP | A | 114 | 29.718 | 111.023 | 175.268 | 1.00 | 34.68 |
| 926 | N | VAL | A | 115 | 28.967 | 110.858 | 173.148 | 1.00 | 30.06 |
| 927 | CA | VAL | A | 115 | 30.048 | 109.990 | 172.687 | 1.00 | 31.75 |
| 928 | CB | VAL | A | 115 | 29.579 | 108.551 | 172.316 | 1.00 | 9.73 |
| 929 | CG1 | VAL | A | 115 | 30.611 | 107.900 | 171.442 | 1.00 | 60.47 |
| 930 | CG2 | VAL | A | 115 | 29.447 | 107.693 | 173.535 | 1.00 | 5.42 |
| 931 | C | VAL | A | 115 | 30.623 | 110.612 | 171.426 | 1.00 | 24.47 |
| 932 | O | VAL | A | 115 | 29.931 | 110.735 | 170.423 | 1.00 | 47.65 |
| 933 | N | TYR | A | 116 | 31.886 | 111.012 | 171.465 | 1.00 | 30.10 |
| 934 | CA | TYR | A | 116 | 32.479 | 111.602 | 170.279 | 1.00 | 17.95 |
| 935 | CB | TYR | A | 116 | 33.256 | 112.865 | 170.637 | 1.00 | 61.65 |
| 936 | CG | TYR | A | 116 | 32.427 | 113.950 | 171.286 | 1.00 | 16.29 |
| 937 | CD1 | TYR | A | 116 | 32.051 | 113.862 | 172.611 | 1.00 | 20.60 |
| 938 | CE1 | TYR | A | 116 | 31.273 | 114.839 | 173.205 | 1.00 | 49.07 |
| 939 | CD2 | TYR | A | 116 | 32.004 | 115.049 | 170.560 | 1.00 | 22.42 |
| 940 | CE2 | TYR | A | 116 | 31.227 | 116.031 | 171.138 | 1.00 | 73.73 |
| 941 | CZ | TYR | A | 116 | 30.864 | 115.920 | 172.460 | 1.00 | 14.21 |
| 942 | OH | TYR | A | 116 | 30.091 | 116.898 | 173.033 | 1.00 | 94.60 |
| 943 | C | TYR | A | 116 | 33.391 | 110.599 | 169.588 | 1.00 | 45.63 |
| 944 | O | TYR | A | 116 | 33.850 | 109.628 | 170.205 | 1.00 | 28.02 |
| 945 | N | LYS | A | 117 | 33.636 | 110.842 | 168.302 | 1.00 | 6.49 |
| 946 | CA | LYS | A | 117 | 34.479 | 109.975 | 167.479 | 1.00 | 35.81 |
| 947 | CB | LYS | A | 117 | 35.937 | 110.034 | 167.974 | 1.00 | 21.69 |
| 948 | CG | LYS | A | 117 | 36.715 | 111.214 | 167.404 | 1.00 | 9.02 |
| 949 | CD | LYS | A | 117 | 37.800 | 111.699 | 168.323 | 1.00 | 36.98 |
| 950 | CE | LYS | A | 117 | 38.449 | 112.977 | 167.779 | 1.00 | 25.36 |
| 951 | NZ | LYS | A | 117 | 39.653 | 113.406 | 168.569 | 1.00 | 25.77 |
| 952 | C | LYS | A | 117 | 33.962 | 108.543 | 167.472 | 1.00 | 7.50 |
| 953 | O | LYS | A | 117 | 34.687 | 107.595 | 167.749 | 1.00 | 52.96 |
| 954 | N | VAL | A | 118 | 32.693 | 108.398 | 167.136 | 1.00 | 46.87 |
| 955 | CA | VAL | A | 118 | 32.045 | 107.102 | 167.109 | 1.00 | 14.75 |
| 956 | CB | VAL | A | 118 | 30.540 | 107.259 | 167.103 | 1.00 | 8.67 |
| 957 | CG1 | VAL | A | 118 | 29.897 | 105.947 | 167.298 | 1.00 | 27.58 |
| 958 | CG2 | VAL | A | 118 | 30.122 | 108.227 | 168.165 | 1.00 | 69.02 |
| 959 | C | VAL | A | 118 | 32.404 | 106.398 | 165.829 | 1.00 | 30.61 |
| 960 | O | VAL | A | 118 | 32.314 | 106.981 | 164.755 | 1.00 | 19.62 |
| 961 | N | ILE | A | 119 | 32.821 | 105.148 | 165.939 | 1.00 | 26.47 |
| 962 | CA | ILE | A | 119 | 33.130 | 104.369 | 164.753 | 1.00 | 43.90 |
| 963 | CB | ILE | A | 119 | 34.604 | 104.042 | 164.634 | 1.00 | 35.19 |
| 964 | CG2 | ILE | A | 119 | 34.809 | 103.093 | 163.482 | 1.00 | 23.28 |
| 965 | CG1 | ILE | A | 119 | 35.406 | 105.305 | 164.379 | 1.00 | 48.21 |
| 966 | CD1 | ILE | A | 119 | 36.855 | 105.042 | 164.194 | 1.00 | 13.69 |
| 967 | C | ILE | A | 119 | 32.391 | 103.054 | 164.815 | 1.00 | 59.19 |
| 968 | O | ILE | A | 119 | 32.403 | 102.371 | 165.840 | 1.00 | 67.21 |
| 969 | N | TYR | A | 120 | 31.743 | 102.698 | 163.720 | 1.00 | 17.08 |
| 970 | CA | TYR | A | 120 | 31.023 | 101.445 | 163.679 | 1.00 | 30.07 |
| 971 | CB | TYR | A | 120 | 29.692 | 101.622 | 162.976 | 1.00 | 13.53 |
| 972 | CG | TYR | A | 120 | 28.673 | 102.393 | 163.755 | 1.00 | 11.12 |
| 973 | CD1 | TYR | A | 120 | 28.517 | 103.760 | 163.585 | 1.00 | 17.26 |
| 974 | CE1 | TYR | A | 120 | 27.509 | 104.438 | 164.227 | 1.00 | 5.42 |
| 975 | CD2 | TYR | A | 120 | 27.806 | 101.737 | 164.599 | 1.00 | 25.25 |
| 976 | CE2 | TYR | A | 120 | 26.803 | 102.398 | 165.243 | 1.00 | 15.18 |
| 977 | CZ | TYR | A | 120 | 26.647 | 103.740 | 165.056 | 1.00 | 46.64 |
| 978 | OH | TYR | A | 120 | 25.595 | 104.357 | 165.687 | 1.00 | 52.40 |
| 979 | C | TYR | A | 120 | 31.864 | 100.435 | 162.917 | 1.00 | 68.41 |
| 980 | O | TYR | A | 120 | 32.395 | 100.743 | 161.850 | 1.00 | 28.49 |
| 981 | N | TYR | A | 121 | 31.991 | 99.229 | 163.466 | 1.00 | 51.87 |
| 982 | CA | TYR | A | 121 | 32.769 | 98.187 | 162.811 | 1.00 | 25.77 |
| 983 | CB | TYR | A | 121 | 33.826 | 97.598 | 163.747 | 1.00 | 54.55 |
| 984 | CG | TYR | A | 121 | 34.978 | 98.529 | 164.015 | 1.00 | 8.44 |
| 985 | CD1 | TYR | A | 121 | 34.991 | 99.343 | 165.133 | 1.00 | 59.81 |
| 986 | CE1 | TYR | A | 121 | 36.010 | 100.254 | 165.343 | 1.00 | 105.65 |
| 987 | CD2 | TYR | A | 121 | 36.022 | 98.645 | 163.112 | 1.00 | 79.41 |
| 988 | CE2 | TYR | A | 121 | 37.047 | 99.558 | 163.313 | 1.00 | 65.81 |
| 989 | CZ | TYR | A | 121 | 37.031 | 100.355 | 164.426 | 1.00 | 13.26 |
| 990 | OH | TYR | A | 121 | 38.023 | 101.270 | 164.627 | 1.00 | 74.37 |
| 991 | C | TYR | A | 121 | 31.882 | 97.078 | 162.315 | 1.00 | 71.36 |
| 992 | O | TYR | A | 121 | 30.877 | 96.730 | 162.942 | 1.00 | 47.92 |
| 993 | N | LYS | A | 122 | 32.271 | 96.533 | 161.171 | 1.00 | 48.07 |
| 994 | CA | LYS | A | 122 | 31.550 | 95.452 | 160.537 | 1.00 | 62.27 |
| 995 | CB | LYS | A | 122 | 30.826 | 95.951 | 159.290 | 1.00 | 82.65 |
| 996 | CG | LYS | A | 122 | 30.100 | 94.878 | 158.498 | 1.00 | 56.59 |
| 997 | CD | LYS | A | 122 | 29.471 | 95.512 | 157.272 | 1.00 | 114.33 |
| 998 | CE | LYS | A | 122 | 28.714 | 94.519 | 156.423 | 1.00 | 90.73 |
| 999 | NZ | LYS | A | 122 | 28.074 | 95.227 | 155.275 | 1.00 | 102.70 |
| 1000 | C | LYS | A | 122 | 32.575 | 94.415 | 160.149 | 1.00 | 51.78 |
| 1001 | O | LYS | A | 122 | 33.377 | 94.622 | 159.236 | 1.00 | 58.80 |
| 1002 | N | ASP | A | 123 | 32.544 | 93.296 | 160.855 | 1.00 | 69.12 |
| 1003 | CA | ASP | A | 123 | 33.464 | 92.205 | 160.595 | 1.00 | 94.48 |
| 1004 | CB | ASP | A | 123 | 33.175 | 91.559 | 159.230 | 1.00 | 102.02 |
| 1005 | CG | ASP | A | 123 | 31.808 | 90.890 | 159.170 | 1.00 | 106.91 |
| 1006 | OD1 | ASP | A | 123 | 31.492 | 90.097 | 160.087 | 1.00 | 80.34 |
| 1007 | OD2 | ASP | A | 123 | 31.056 | 91.152 | 158.201 | 1.00 | 91.23 |
| 1008 | C | ASP | A | 123 | 34.887 | 92.725 | 160.633 | 1.00 | 42.69 |
| 1009 | O | ASP | A | 123 | 35.642 | 92.572 | 159.682 | 1.00 | 80.39 |
| 1010 | N | GLY | A | 124 | 35.240 | 93.353 | 161.741 | 1.00 | 42.82 |
| 1011 | CA | GLY | A | 124 | 36.585 | 93.862 | 161.892 | 1.00 | 58.87 |
| 1012 | C | GLY | A | 124 | 36.987 | 94.991 | 160.970 | 1.00 | 46.57 |
| 1013 | O | GLY | A | 124 | 38.117 | 95.452 | 161.037 | 1.00 | 81.66 |
| 1014 | N | GLU | A | 125 | 36.092 | 95.440 | 160.102 | 1.00 | 57.50 |
| 1015 | CA | GLU | A | 125 | 36.434 | 96.544 | 159.211 | 1.00 | 57.91 |
| 1016 | CB | GLU | A | 125 | 35.933 | 96.298 | 157.791 | 1.00 | 140.86 |
| 1017 | CG | GLU | A | 125 | 36.385 | 95.024 | 157.122 | 1.00 | 176.84 |
| 1018 | CD | GLU | A | 125 | 35.928 | 94.972 | 155.677 | 1.00 | 189.28 |
| 1019 | OE1 | GLU | A | 125 | 34.704 | 95.097 | 155.438 | 1.00 | 165.09 |
| 1020 | OE2 | GLU | A | 125 | 36.794 | 94.814 | 154.785 | 1.00 | 176.42 |
| 1021 | C | GLU | A | 125 | 35.779 | 97.823 | 159.701 | 1.00 | 49.34 |
| 1022 | O | GLU | A | 125 | 34.723 | 97.791 | 160.328 | 1.00 | 70.32 |
| 1023 | N | ALA | A | 126 | 36.400 | 98.953 | 159.400 | 1.00 | 54.71 |
| 1024 | CA | ALA | A | 126 | 35.850 | 100.237 | 159.793 | 1.00 | 36.67 |
| 1025 | CB | ALA | A | 126 | 36.928 | 101.281 | 159.793 | 1.00 | 40.73 |
| 1026 | C | ALA | A | 126 | 34.775 | 100.614 | 158.799 | 1.00 | 28.44 |
| 1027 | O | ALA | A | 126 | 35.074 | 100.962 | 157.660 | 1.00 | 49.69 |
| 1028 | N | LEU | A | 127 | 33.523 | 100.557 | 159.235 | 1.00 | 38.11 |
| 1029 | CA | LEU | A | 127 | 32.412 | 100.888 | 158.365 | 1.00 | 35.41 |
| 1030 | CB | LEU | A | 127 | 31.141 | 100.219 | 158.880 | 1.00 | 24.22 |
| 1031 | CG | LEU | A | 127 | 29.869 | 100.320 | 158.045 | 1.00 | 33.49 |
| 1032 | CD1 | LEU | A | 127 | 30.194 | 100.562 | 156.587 | 1.00 | 56.43 |
| 1033 | CD2 | LEU | A | 127 | 29.077 | 99.041 | 158.237 | 1.00 | 47.01 |
| 1034 | C | LEU | A | 127 | 32.226 | 102.393 | 158.225 | 1.00 | 30.36 |
| 1035 | O | LEU | A | 127 | 32.289 | 102.902 | 157.118 | 1.00 | 60.67 |
| 1036 | N | LYS | A | 128 | 32.014 | 103.108 | 159.331 | 1.00 | 28.99 |
| 1037 | CA | LYS | A | 128 | 31.836 | 104.566 | 159.273 | 1.00 | 33.91 |
| 1038 | CB | LYS | A | 128 | 30.366 | 104.916 | 159.041 | 1.00 | 38.64 |
| 1039 | CG | LYS | A | 128 | 30.131 | 106.337 | 158.560 | 1.00 | 15.25 |
| 1040 | CD | LYS | A | 128 | 28.691 | 106.479 | 158.073 | 1.00 | 113.62 |
| 1041 | CE | LYS | A | 128 | 28.493 | 107.706 | 157.188 | 1.00 | 123.73 |
| 1042 | NZ | LYS | A | 128 | 27.202 | 107.663 | 156.419 | 1.00 | 121.55 |
| 1043 | C | LYS | A | 128 | 32.317 | 105.281 | 160.530 | 1.00 | 52.08 |
| 1044 | O | LYS | A | 128 | 32.077 | 104.819 | 161.647 | 1.00 | 65.58 |
| 1045 | N | TYR | A | 129 | 33.011 | 106.401 | 160.343 | 1.00 | 31.59 |
| 1046 | CA | TYR | A | 129 | 33.509 | 107.213 | 161.459 | 1.00 | 36.12 |
| 1047 | CB | TYR | A | 129 | 35.037 | 107.317 | 161.450 | 1.00 | 18.41 |
| 1048 | CG | TYR | A | 129 | 35.608 | 108.593 | 162.076 | 1.00 | 5.42 |
| 1049 | CD1 | TYR | A | 129 | 36.136 | 108.584 | 163.348 | 1.00 | 44.66 |
| 1050 | CE1 | TYR | A | 129 | 36.655 | 109.734 | 163.923 | 1.00 | 33.53 |
| 1051 | CD2 | TYR | A | 129 | 35.619 | 109.804 | 161.386 | 1.00 | 23.01 |
| 1052 | CE2 | TYR | A | 129 | 36.141 | 110.962 | 161.959 | 1.00 | 5.42 |
| 1053 | CZ | TYR | A | 129 | 36.653 | 110.910 | 163.229 | 1.00 | 39.44 |
| 1054 | OH | TYR | A | 129 | 37.173 | 112.024 | 163.828 | 1.00 | 26.10 |
| 1055 | C | TYR | A | 129 | 32.942 | 108.600 | 161.285 | 1.00 | 49.76 |
| 1056 | O | TYR | A | 129 | 32.749 | 109.063 | 160.160 | 1.00 | 59.27 |
| 1057 | N | TRP | A | 130 | 32.696 | 109.263 | 162.401 | 1.00 | 24.05 |
| 1058 | CA | TRP | A | 130 | 32.177 | 110.612 | 162.378 | 1.00 | 29.33 |
| 1059 | CB | TRP | A | 130 | 30.653 | 110.586 | 162.363 | 1.00 | 41.56 |
| 1060 | CG | TRP | A | 130 | 30.067 | 111.918 | 162.139 | 1.00 | 34.89 |
| 1061 | CD2 | TRP | A | 130 | 30.311 | 112.772 | 161.028 | 1.00 | 20.74 |
| 1062 | CE2 | TRP | A | 130 | 29.535 | 113.931 | 161.210 | 1.00 | 30.33 |
| 1063 | CE3 | TRP | A | 130 | 31.109 | 112.673 | 159.893 | 1.00 | 40.31 |
| 1064 | CD1 | TRP | A | 130 | 29.177 | 112.571 | 162.937 | 1.00 | 110.45 |
| 1065 | NE1 | TRP | A | 130 | 28.849 | 113.786 | 162.385 | 1.00 | 102.78 |
| 1066 | CZ2 | TRP | A | 130 | 29.540 | 114.979 | 160.298 | 1.00 | 108.00 |
| 1067 | CZ3 | TRP | A | 130 | 31.112 | 113.715 | 158.989 | 1.00 | 14.76 |
| 1068 | CH2 | TRP | A | 130 | 30.337 | 114.847 | 159.193 | 1.00 | 47.04 |
| 1069 | C | TRP | A | 130 | 32.700 | 111.316 | 163.628 | 1.00 | 59.29 |
| 1070 | O | TRP | A | 130 | 32.949 | 110.683 | 164.672 | 1.00 | 22.78 |
| 1071 | N | TYR | A | 131 | 32.875 | 112.625 | 163.529 | 1.00 | 5.42 |
| 1072 | CA | TYR | A | 131 | 33.406 | 113.361 | 164.664 | 1.00 | 43.44 |
| 1073 | CB | TYR | A | 131 | 33.533 | 114.856 | 164.336 | 1.00 | 20.56 |
| 1074 | CG | TYR | A | 131 | 34.068 | 115.149 | 162.945 | 1.00 | 6.92 |
| 1075 | CD1 | TYR | A | 131 | 33.219 | 115.167 | 161.850 | 1.00 | 40.67 |
| 1076 | CE1 | TYR | A | 131 | 33.693 | 115.445 | 160.573 | 1.00 | 38.77 |
| 1077 | CD2 | TYR | A | 131 | 35.422 | 115.414 | 162.728 | 1.00 | 31.32 |
| 1078 | CE2 | TYR | A | 131 | 35.908 | 115.689 | 161.455 | 1.00 | 16.93 |
| 1079 | CZ | TYR | A | 131 | 35.034 | 115.708 | 160.384 | 1.00 | 49.10 |
| 1080 | OH | TYR | A | 131 | 35.486 | 116.022 | 159.126 | 1.00 | 47.13 |
| 1081 | C | TYR | A | 131 | 32.535 | 113.175 | 165.887 | 1.00 | 28.76 |
| 1082 | O | TYR | A | 131 | 33.030 | 113.124 | 167.015 | 1.00 | 36.69 |
| 1083 | N | GLU | A | 132 | 31.232 | 113.060 | 165.646 | 1.00 | 21.77 |
| 1084 | CA | GLU | A | 132 | 30.249 | 112.914 | 166.710 | 1.00 | 35.07 |
| 1085 | CB | GLU | A | 132 | 29.315 | 114.113 | 166.691 | 1.00 | 5.42 |
| 1086 | CG | GLU | A | 132 | 29.974 | 115.382 | 167.187 | 1.00 | 32.08 |
| 1087 | CD | GLU | A | 132 | 30.190 | 116.404 | 166.099 | 1.00 | 77.18 |
| 1088 | OE1 | GLU | A | 132 | 29.797 | 116.117 | 164.946 | 1.00 | 30.77 |
| 1089 | OE2 | GLU | A | 132 | 30.748 | 117.487 | 166.408 | 1.00 | 46.31 |
| 1090 | C | GLU | A | 132 | 29.443 | 111.640 | 166.590 | 1.00 | 26.10 |
| 1091 | O | GLU | A | 132 | 29.647 | 110.866 | 165.680 | 1.00 | 44.94 |
| 1092 | N | ASN | A | 133 | 28.529 | 111.413 | 167.518 | 1.00 | 42.20 |
| 1093 | CA | ASN | A | 133 | 27.708 | 110.215 | 167.468 | 1.00 | 5.42 |
| 1094 | CB | ASN | A | 133 | 26.848 | 110.093 | 168.700 | 1.00 | 21.79 |
| 1095 | CG | ASN | A | 133 | 26.554 | 108.677 | 169.032 | 1.00 | 28.76 |
| 1096 | OD1 | ASN | A | 133 | 26.285 | 107.878 | 168.144 | 1.00 | 22.35 |
| 1097 | ND2 | ASN | A | 133 | 26.603 | 108.344 | 170.318 | 1.00 | 74.20 |
| 1098 | C | ASN | A | 133 | 26.811 | 110.371 | 166.285 | 1.00 | 11.75 |
| 1099 | O | ASN | A | 133 | 26.539 | 111.500 | 165.877 | 1.00 | 33.27 |
| 1100 | N | HIS | A | 134 | 26.338 | 109.252 | 165.744 | 1.00 | 5.42 |
| 1101 | CA | HIS | A | 134 | 25.485 | 109.278 | 164.559 | 1.00 | 30.76 |
| 1102 | CB | HIS | A | 134 | 26.317 | 109.594 | 163.321 | 1.00 | 50.57 |
| 1103 | CG | HIS | A | 134 | 27.435 | 108.627 | 163.085 | 1.00 | 21.60 |
| 1104 | CD2 | HIS | A | 134 | 27.659 | 107.753 | 162.077 | 1.00 | 76.57 |
| 1105 | ND1 | HIS | A | 134 | 28.481 | 108.468 | 163.965 | 1.00 | 61.39 |
| 1106 | CE1 | HIS | A | 134 | 29.300 | 107.539 | 163.512 | 1.00 | 26.75 |
| 1107 | NE2 | HIS | A | 134 | 28.823 | 107.088 | 162.368 | 1.00 | 47.66 |
| 1108 | C | HIS | A | 134 | 24.864 | 107.917 | 164.384 | 1.00 | 24.80 |
| 1109 | O | HIS | A | 134 | 25.441 | 106.932 | 164.821 | 1.00 | 42.22 |
| 1110 | N | ALA | A | 135 | 23.706 | 107.844 | 163.733 | 1.00 | 51.38 |
| 1111 | CA | ALA | A | 135 | 23.053 | 106.550 | 163.541 | 1.00 | 50.85 |
| 1112 | CB | ALA | A | 135 | 21.551 | 106.694 | 163.647 | 1.00 | 25.54 |
| 1113 | C | ALA | A | 135 | 23.428 | 106.009 | 162.188 | 1.00 | 30.29 |
| 1114 | O | ALA | A | 135 | 23.718 | 106.776 | 161.277 | 1.00 | 75.67 |
| 1115 | N | ILE | A | 136 | 23.448 | 104.687 | 162.064 | 1.00 | 48.62 |
| 1116 | CA | ILE | A | 136 | 23.789 | 104.039 | 160.802 | 1.00 | 34.81 |
| 1117 | CB | ILE | A | 136 | 24.593 | 102.724 | 160.998 | 1.00 | 59.15 |
| 1118 | CG2 | ILE | A | 136 | 24.856 | 102.076 | 159.664 | 1.00 | 5.42 |
| 1119 | CG1 | ILE | A | 136 | 25.942 | 103.004 | 161.664 | 1.00 | 55.26 |
| 1120 | CD1 | ILE | A | 136 | 26.878 | 103.861 | 160.822 | 1.00 | 92.37 |
| 1121 | C | ILE | A | 136 | 22.450 | 103.677 | 160.245 | 1.00 | 36.44 |
| 1122 | O | ILE | A | 136 | 21.735 | 102.876 | 160.844 | 1.00 | 40.66 |
| 1123 | N | SER | A | 137 | 22.104 | 104.274 | 159.110 | 1.00 | 71.47 |
| 1124 | CA | SER | A | 137 | 20.813 | 104.022 | 158.477 | 1.00 | 77.26 |
| 1125 | CB | SER | A | 137 | 20.064 | 105.335 | 158.248 | 1.00 | 65.71 |
| 1126 | OG | SER | A | 137 | 19.920 | 106.070 | 159.449 | 1.00 | 92.34 |
| 1127 | C | SER | A | 137 | 20.952 | 103.301 | 157.150 | 1.00 | 55.90 |
| 1128 | O | SER | A | 137 | 21.323 | 103.895 | 156.137 | 1.00 | 68.68 |
| 1129 | N | ILE | A | 138 | 20.650 | 102.012 | 157.164 | 1.00 | 62.52 |
| 1130 | CA | ILE | A | 138 | 20.717 | 101.202 | 155.961 | 1.00 | 61.92 |
| 1131 | CB | ILE | A | 138 | 21.275 | 99.827 | 156.270 | 1.00 | 45.81 |
| 1132 | CG2 | ILE | A | 138 | 21.312 | 98.999 | 155.015 | 1.00 | 82.72 |
| 1133 | CG1 | ILE | A | 138 | 22.673 | 99.965 | 156.863 | 1.00 | 76.93 |
| 1134 | CD1 | ILE | A | 138 | 23.230 | 98.666 | 157.415 | 1.00 | 63.77 |
| 1135 | C | ILE | A | 138 | 19.312 | 101.056 | 155.375 | 1.00 | 89.17 |
| 1136 | O | ILE | A | 138 | 18.525 | 100.208 | 155.807 | 1.00 | 38.61 |
| 1137 | N | THR | A | 139 | 19.023 | 101.907 | 154.393 | 1.00 | 109.71 |
| 1138 | CA | THR | A | 139 | 17.742 | 101.970 | 153.689 | 1.00 | 95.12 |
| 1139 | CB | THR | A | 139 | 17.840 | 102.956 | 152.539 | 1.00 | 101.48 |
| 1140 | OG1 | THR | A | 139 | 19.147 | 102.850 | 151.946 | 1.00 | 113.46 |
| 1141 | CG2 | THR | A | 139 | 17.596 | 104.370 | 153.036 | 1.00 | 70.63 |
| 1142 | C | THR | A | 139 | 17.208 | 100.663 | 153.123 | 1.00 | 107.81 |
| 1143 | O | THR | A | 139 | 16.044 | 100.321 | 153.342 | 1.00 | 75.17 |
| 1144 | N | ASN | A | 140 | 18.048 | 99.955 | 152.370 | 1.00 | 119.96 |
| 1145 | CA | ASN | A | 140 | 17.660 | 98.679 | 151.769 | 1.00 | 77.07 |
| 1146 | CB | ASN | A | 140 | 17.495 | 98.828 | 150.257 | 1.00 | 128.43 |
| 1147 | CG | ASN | A | 140 | 16.465 | 99.873 | 149.888 | 1.00 | 149.94 |
| 1148 | OD1 | ASN | A | 140 | 15.330 | 99.840 | 150.365 | 1.00 | 158.03 |
| 1149 | ND2 | ASN | A | 140 | 16.855 | 100.810 | 149.029 | 1.00 | 147.94 |
| 1150 | C | ASN | A | 140 | 18.691 | 97.603 | 152.065 | 1.00 | 96.65 |
| 1151 | O | ASN | A | 140 | 19.802 | 97.615 | 151.527 | 1.00 | 86.22 |
| 1152 | N | ALA | A | 141 | 18.308 | 96.666 | 152.922 | 1.00 | 38.12 |
| 1153 | CA | ALA | A | 141 | 19.196 | 95.588 | 153.316 | 1.00 | 94.25 |
| 1154 | CB | ALA | A | 141 | 18.435 | 94.592 | 154.164 | 1.00 | 53.34 |
| 1155 | C | ALA | A | 141 | 19.844 | 94.885 | 152.126 | 1.00 | 79.54 |
| 1156 | O | ALA | A | 141 | 19.409 | 95.042 | 150.987 | 1.00 | 77.48 |
| 1157 | N | ALA | A | 142 | 20.889 | 94.112 | 152.406 | 1.00 | 90.86 |
| 1158 | CA | ALA | A | 142 | 21.617 | 93.374 | 151.383 | 1.00 | 76.61 |
| 1159 | CB | ALA | A | 142 | 22.683 | 94.261 | 150.761 | 1.00 | 121.64 |
| 1160 | C | ALA | A | 142 | 22.260 | 92.150 | 152.022 | 1.00 | 74.42 |
| 1161 | O | ALA | A | 142 | 21.987 | 91.843 | 153.176 | 1.00 | 111.57 |
| 1162 | N | VAL | A | 143 | 23.114 | 91.458 | 151.274 | 1.00 | 108.94 |
| 1163 | CA | VAL | A | 143 | 23.795 | 90.262 | 151.768 | 1.00 | 116.05 |
| 1164 | CB | VAL | A | 143 | 24.071 | 89.259 | 150.609 | 1.00 | 186.06 |
| 1165 | CG1 | VAL | A | 143 | 24.745 | 89.977 | 149.434 | 1.00 | 191.15 |
| 1166 | CG2 | VAL | A | 143 | 24.948 | 88.112 | 151.109 | 1.00 | 210.48 |
| 1167 | C | VAL | A | 143 | 25.115 | 90.625 | 152.440 | 1.00 | 117.36 |
| 1168 | O | VAL | A | 143 | 25.496 | 90.038 | 153.456 | 1.00 | 94.53 |
| 1169 | N | GLU | A | 144 | 25.813 | 91.588 | 151.853 | 1.00 | 118.63 |
| 1170 | CA | GLU | A | 144 | 27.080 | 92.049 | 152.389 | 1.00 | 89.21 |
| 1171 | CB | GLU | A | 144 | 27.662 | 93.116 | 151.474 | 1.00 | 107.26 |
| 1172 | CG | GLU | A | 144 | 26.646 | 94.190 | 151.126 | 1.00 | 114.43 |
| 1173 | CD | GLU | A | 144 | 27.276 | 95.427 | 150.535 | 1.00 | 150.22 |
| 1174 | OE1 | GLU | A | 144 | 28.027 | 95.290 | 149.546 | 1.00 | 173.95 |
| 1175 | OE2 | GLU | A | 144 | 27.015 | 96.535 | 151.056 | 1.00 | 124.46 |
| 1176 | C | GLU | A | 144 | 26.810 | 92.655 | 153.756 | 1.00 | 100.45 |
| 1177 | O | GLU | A | 144 | 27.642 | 92.564 | 154.661 | 1.00 | 113.43 |
| 1178 | N | ASP | A | 145 | 25.635 | 93.269 | 153.891 | 1.00 | 69.60 |
| 1179 | CA | ASP | A | 145 | 25.231 | 93.912 | 155.135 | 1.00 | 62.50 |
| 1180 | CB | ASP | A | 145 | 23.927 | 94.699 | 154.945 | 1.00 | 35.86 |
| 1181 | CG | ASP | A | 145 | 24.153 | 96.053 | 154.284 | 1.00 | 114.94 |
| 1182 | OD1 | ASP | A | 145 | 25.187 | 96.694 | 154.585 | 1.00 | 110.06 |
| 1183 | OD2 | ASP | A | 145 | 23.295 | 96.481 | 153.477 | 1.00 | 95.06 |
| 1184 | C | ASP | A | 145 | 25.102 | 93.000 | 156.350 | 1.00 | 52.48 |
| 1185 | O | ASP | A | 145 | 24.774 | 93.466 | 157.435 | 1.00 | 45.00 |
| 1186 | N | SER | A | 146 | 25.339 | 91.706 | 156.189 | 1.00 | 41.79 |
| 1187 | CA | SER | A | 146 | 25.277 | 90.834 | 157.347 | 1.00 | 56.05 |
| 1188 | CB | SER | A | 146 | 24.848 | 89.426 | 156.965 | 1.00 | 71.16 |
| 1189 | OG | SER | A | 146 | 23.440 | 89.341 | 156.889 | 1.00 | 94.99 |
| 1190 | C | SER | A | 146 | 26.658 | 90.785 | 157.969 | 1.00 | 69.92 |
| 1191 | O | SER | A | 146 | 27.594 | 91.438 | 157.498 | 1.00 | 69.56 |
| 1192 | N | GLY | A | 147 | 26.787 | 90.000 | 159.026 | 1.00 | 47.55 |
| 1193 | CA | GLY | A | 147 | 28.067 | 89.894 | 159.693 | 1.00 | 98.06 |
| 1194 | C | GLY | A | 147 | 27.921 | 90.473 | 161.076 | 1.00 | 50.55 |
| 1195 | O | GLY | A | 147 | 26.837 | 90.915 | 161.439 | 1.00 | 81.86 |
| 1196 | N | THR | A | 148 | 28.996 | 90.477 | 161.852 | 1.00 | 54.75 |
| 1197 | CA | THR | A | 148 | 28.923 | 91.009 | 163.199 | 1.00 | 47.04 |
| 1198 | CB | THR | A | 148 | 29.784 | 90.196 | 164.160 | 1.00 | 5.42 |
| 1199 | OG1 | THR | A | 148 | 31.016 | 90.882 | 164.383 | 1.00 | 70.34 |
| 1200 | CG2 | THR | A | 148 | 30.080 | 88.841 | 163.572 | 1.00 | 60.94 |
| 1201 | C | THR | A | 148 | 29.380 | 92.461 | 163.255 | 1.00 | 42.31 |
| 1202 | O | THR | A | 148 | 30.388 | 92.827 | 162.656 | 1.00 | 52.98 |
| 1203 | N | TYR | A | 149 | 28.623 | 93.285 | 163.976 | 1.00 | 52.62 |
| 1204 | CA | TYR | A | 149 | 28.950 | 94.695 | 164.128 | 1.00 | 25.73 |
| 1205 | CB | TYR | A | 149 | 27.786 | 95.584 | 163.725 | 1.00 | 15.90 |
| 1206 | CG | TYR | A | 149 | 27.380 | 95.588 | 162.288 | 1.00 | 26.25 |
| 1207 | CD1 | TYR | A | 149 | 26.653 | 94.538 | 161.750 | 1.00 | 26.15 |
| 1208 | CE1 | TYR | A | 149 | 26.201 | 94.583 | 160.445 | 1.00 | 48.47 |
| 1209 | CD2 | TYR | A | 149 | 27.651 | 96.686 | 161.482 | 1.00 | 25.17 |
| 1210 | CE2 | TYR | A | 149 | 27.206 | 96.742 | 160.182 | 1.00 | 41.99 |
| 1211 | CZ | TYR | A | 149 | 26.483 | 95.688 | 159.666 | 1.00 | 45.90 |
| 1212 | OH | TYR | A | 149 | 26.063 | 95.738 | 158.359 | 1.00 | 54.85 |
| 1213 | C | TYR | A | 149 | 29.266 | 95.050 | 165.577 | 1.00 | 59.07 |
| 1214 | O | TYR | A | 149 | 29.000 | 94.275 | 166.503 | 1.00 | 29.97 |
| 1215 | N | TYR | A | 150 | 29.820 | 96.244 | 165.757 | 1.00 | 27.86 |
| 1216 | CA | TYR | A | 150 | 30.127 | 96.766 | 167.078 | 1.00 | 46.90 |
| 1217 | CB | TYR | A | 150 | 31.126 | 95.863 | 167.799 | 1.00 | 32.05 |
| 1218 | CG | TYR | A | 150 | 32.540 | 95.971 | 167.323 | 1.00 | 33.00 |
| 1219 | CD1 | TYR | A | 150 | 33.391 | 96.933 | 167.837 | 1.00 | 32.03 |
| 1220 | CE1 | TYR | A | 150 | 34.704 | 97.018 | 167.419 | 1.00 | 59.03 |
| 1221 | CD2 | TYR | A | 150 | 33.038 | 95.093 | 166.369 | 1.00 | 75.94 |
| 1222 | CE2 | TYR | A | 150 | 34.349 | 95.169 | 165.943 | 1.00 | 69.80 |
| 1223 | CZ | TYR | A | 150 | 35.177 | 96.135 | 166.473 | 1.00 | 46.28 |
| 1224 | OH | TYR | A | 150 | 36.476 | 96.223 | 166.049 | 1.00 | 71.42 |
| 1225 | C | TYR | A | 150 | 30.667 | 98.182 | 166.902 | 1.00 | 52.29 |
| 1226 | O | TYR | A | 150 | 31.175 | 98.516 | 165.836 | 1.00 | 49.86 |
| 1227 | N | CYS | A | 151 | 30.517 | 99.025 | 167.923 | 1.00 | 31.32 |
| 1228 | CA | CYS | A | 151 | 30.989 | 100.393 | 167.832 | 1.00 | 7.95 |
| 1229 | C | CYS | A | 151 | 31.968 | 100.769 | 168.914 | 1.00 | 40.57 |
| 1230 | O | CYS | A | 151 | 32.057 | 100.120 | 169.946 | 1.00 | 51.30 |
| 1231 | CB | CYS | A | 151 | 29.813 | 101.372 | 167.856 | 1.00 | 53.04 |
| 1232 | SG | CYS | A | 151 | 28.694 | 101.368 | 169.294 | 1.00 | 69.58 |
| 1233 | N | THR | A | 152 | 32.708 | 101.835 | 168.663 | 1.00 | 26.38 |
| 1234 | CA | THR | A | 152 | 33.671 | 102.341 | 169.617 | 1.00 | 14.07 |
| 1235 | CB | THR | A | 152 | 35.085 | 102.028 | 169.185 | 1.00 | 22.97 |
| 1236 | OG1 | THR | A | 152 | 35.335 | 102.632 | 167.916 | 1.00 | 43.42 |
| 1237 | CG2 | THR | A | 152 | 35.276 | 100.549 | 169.055 | 1.00 | 54.46 |
| 1238 | C | THR | A | 152 | 33.489 | 103.840 | 169.620 | 1.00 | 59.80 |
| 1239 | O | THR | A | 152 | 32.993 | 104.417 | 168.645 | 1.00 | 26.00 |
| 1240 | N | GLY | A | 153 | 33.890 | 104.471 | 170.715 | 1.00 | 24.03 |
| 1241 | CA | GLY | A | 153 | 33.751 | 105.909 | 170.812 | 1.00 | 38.00 |
| 1242 | C | GLY | A | 153 | 34.368 | 106.444 | 172.082 | 1.00 | 50.23 |
| 1243 | O | GLY | A | 153 | 34.734 | 105.680 | 172.984 | 1.00 | 10.68 |
| 1244 | N | LYS | A | 154 | 34.497 | 107.762 | 172.155 | 1.00 | 20.76 |
| 1245 | CA | LYS | A | 154 | 35.071 | 108.364 | 173.336 | 1.00 | 22.57 |
| 1246 | CB | LYS | A | 154 | 36.150 | 109.375 | 172.947 | 1.00 | 18.43 |
| 1247 | CG | LYS | A | 154 | 37.401 | 108.744 | 172.357 | 1.00 | 68.32 |
| 1248 | CD | LYS | A | 154 | 38.459 | 109.762 | 171.955 | 1.00 | 79.74 |
| 1249 | CE | LYS | A | 154 | 39.759 | 109.070 | 171.557 | 1.00 | 96.73 |
| 1250 | NZ | LYS | A | 154 | 40.839 | 110.040 | 171.221 | 1.00 | 103.77 |
| 1251 | C | LYS | A | 154 | 33.937 | 109.023 | 174.095 | 1.00 | 49.37 |
| 1252 | O | LYS | A | 154 | 33.075 | 109.656 | 173.498 | 1.00 | 37.93 |
| 1253 | N | VAL | A | 155 | 33.941 | 108.831 | 175.411 | 1.00 | 29.99 |
| 1254 | CA | VAL | A | 155 | 32.935 | 109.355 | 176.314 | 1.00 | 9.34 |
| 1255 | CB | VAL | A | 155 | 32.099 | 108.223 | 176.890 | 1.00 | 21.29 |
| 1256 | CG1 | VAL | A | 155 | 31.048 | 108.748 | 177.826 | 1.00 | 23.90 |
| 1257 | CG2 | VAL | A | 155 | 31.476 | 107.478 | 175.791 | 1.00 | 37.70 |
| 1258 | C | VAL | A | 155 | 33.687 | 109.997 | 177.457 | 1.00 | 27.74 |
| 1259 | O | VAL | A | 155 | 34.312 | 109.306 | 178.262 | 1.00 | 13.56 |
| 1260 | N | TRP | A | 156 | 33.627 | 111.314 | 177.557 | 1.00 | 38.73 |
| 1261 | CA | TRP | A | 156 | 34.351 | 111.977 | 178.633 | 1.00 | 69.64 |
| 1262 | CB | TRP | A | 156 | 33.966 | 111.410 | 179.999 | 1.00 | 28.52 |
| 1263 | CG | TRP | A | 156 | 32.519 | 111.540 | 180.386 | 1.00 | 64.30 |
| 1264 | CD2 | TRP | A | 156 | 31.672 | 112.685 | 180.244 | 1.00 | 16.66 |
| 1265 | CE2 | TRP | A | 156 | 30.417 | 112.342 | 180.786 | 1.00 | 65.75 |
| 1266 | CE3 | TRP | A | 156 | 31.847 | 113.967 | 179.715 | 1.00 | 72.95 |
| 1267 | CD1 | TRP | A | 156 | 31.759 | 110.583 | 180.991 | 1.00 | 80.53 |
| 1268 | NE1 | TRP | A | 156 | 30.500 | 111.052 | 181.237 | 1.00 | 20.03 |
| 1269 | CZ2 | TRP | A | 156 | 29.348 | 113.232 | 180.808 | 1.00 | 14.24 |
| 1270 | CZ3 | TRP | A | 156 | 30.775 | 114.850 | 179.744 | 1.00 | 11.55 |
| 1271 | CH2 | TRP | A | 156 | 29.551 | 114.477 | 180.284 | 1.00 | 40.65 |
| 1272 | C | TRP | A | 156 | 35.834 | 111.721 | 178.407 | 1.00 | 58.02 |
| 1273 | O | TRP | A | 156 | 36.565 | 111.406 | 179.334 | 1.00 | 32.03 |
| 1274 | N | GLN | A | 157 | 36.254 | 111.828 | 177.154 | 1.00 | 29.52 |
| 1275 | CA | GLN | A | 157 | 37.647 | 111.654 | 176.770 | 1.00 | 71.13 |
| 1276 | CB | GLN | A | 157 | 38.515 | 112.648 | 177.532 | 1.00 | 21.16 |
| 1277 | CG | GLN | A | 157 | 38.719 | 113.952 | 176.766 | 1.00 | 47.59 |
| 1278 | CD | GLN | A | 157 | 38.945 | 115.134 | 177.676 | 1.00 | 106.91 |
| 1279 | OE1 | GLN | A | 157 | 39.737 | 115.068 | 178.626 | 1.00 | 62.17 |
| 1280 | NE2 | GLN | A | 157 | 38.254 | 116.237 | 177.390 | 1.00 | 132.54 |
| 1281 | C | GLN | A | 157 | 38.273 | 110.271 | 176.820 | 1.00 | 20.80 |
| 1282 | O | GLN | A | 157 | 39.389 | 110.086 | 176.338 | 1.00 | 50.51 |
| 1283 | N | LEU | A | 158 | 37.569 | 109.299 | 177.380 | 1.00 | 41.89 |
| 1284 | CA | LEU | A | 158 | 38.096 | 107.940 | 177.410 | 1.00 | 24.44 |
| 1285 | CB | LEU | A | 158 | 37.826 | 107.303 | 178.761 | 1.00 | 29.61 |
| 1286 | CG | LEU | A | 158 | 38.450 | 108.159 | 179.850 | 1.00 | 5.42 |
| 1287 | CD1 | LEU | A | 158 | 38.603 | 107.360 | 181.127 | 1.00 | 45.69 |
| 1288 | CD2 | LEU | A | 158 | 39.806 | 108.640 | 179.378 | 1.00 | 68.22 |
| 1289 | C | LEU | A | 158 | 37.525 | 107.073 | 176.281 | 1.00 | 69.39 |
| 1290 | O | LEU | A | 158 | 36.521 | 107.420 | 175.653 | 1.00 | 33.83 |
| 1291 | N | ASP | A | 159 | 38.163 | 105.938 | 176.024 | 1.00 | 33.70 |
| 1292 | CA | ASP | A | 159 | 37.723 | 105.088 | 174.933 | 1.00 | 15.27 |
| 1293 | CB | ASP | A | 159 | 38.940 | 104.625 | 174.126 | 1.00 | 72.08 |
| 1294 | CG | ASP | A | 159 | 39.602 | 105.757 | 173.350 | 1.00 | 44.73 |
| 1295 | OD1 | ASP | A | 159 | 38.896 | 106.450 | 172.601 | 1.00 | 81.88 |
| 1296 | OD2 | ASP | A | 159 | 40.830 | 105.951 | 173.470 | 1.00 | 105.33 |
| 1297 | C | ASP | A | 159 | 36.897 | 103.886 | 175.355 | 1.00 | 50.78 |
| 1298 | O | ASP | A | 159 | 37.192 | 103.224 | 176.356 | 1.00 | 42.36 |
| 1299 | N | TYR | A | 160 | 35.859 | 103.602 | 174.577 | 1.00 | 14.41 |
| 1300 | CA | TYR | A | 160 | 35.006 | 102.466 | 174.875 | 1.00 | 19.18 |
| 1301 | CB | TYR | A | 160 | 33.779 | 102.923 | 175.654 | 1.00 | 30.10 |
| 1302 | CG | TYR | A | 160 | 34.112 | 103.527 | 177.002 | 1.00 | 39.60 |
| 1303 | CD1 | TYR | A | 160 | 34.388 | 104.884 | 177.140 | 1.00 | 9.47 |
| 1304 | CE1 | TYR | A | 160 | 34.750 | 105.418 | 178.371 | 1.00 | 50.45 |
| 1305 | CD2 | TYR | A | 160 | 34.198 | 102.725 | 178.131 | 1.00 | 36.31 |
| 1306 | CE2 | TYR | A | 160 | 34.551 | 103.247 | 179.361 | 1.00 | 31.00 |
| 1307 | CZ | TYR | A | 160 | 34.830 | 104.587 | 179.480 | 1.00 | 32.81 |
| 1308 | OH | TYR | A | 160 | 35.206 | 105.063 | 180.718 | 1.00 | 38.13 |
| 1309 | C | TYR | A | 160 | 34.583 | 101.703 | 173.629 | 1.00 | 62.53 |
| 1310 | O | TYR | A | 160 | 34.549 | 102.246 | 172.520 | 1.00 | 45.60 |
| 1311 | N | GLU | A | 161 | 34.249 | 100.435 | 173.824 | 1.00 | 46.69 |
| 1312 | CA | GLU | A | 161 | 33.852 | 99.563 | 172.727 | 1.00 | 47.10 |
| 1313 | CB | GLU | A | 161 | 35.018 | 98.620 | 172.417 | 1.00 | 44.91 |
| 1314 | CG | GLU | A | 161 | 34.882 | 97.745 | 171.191 | 1.00 | 102.74 |
| 1315 | CD | GLU | A | 161 | 36.108 | 96.855 | 170.973 | 1.00 | 98.25 |
| 1316 | OE1 | GLU | A | 161 | 36.103 | 96.032 | 170.030 | 1.00 | 115.20 |
| 1317 | OE2 | GLU | A | 161 | 37.079 | 96.980 | 171.749 | 1.00 | 83.84 |
| 1318 | C | GLU | A | 161 | 32.617 | 98.782 | 173.181 | 1.00 | 57.73 |
| 1319 | O | GLU | A | 161 | 32.586 | 98.245 | 174.284 | 1.00 | 75.56 |
| 1320 | N | SER | A | 162 | 31.594 | 98.741 | 172.337 | 1.00 | 36.86 |
| 1321 | CA | SER | A | 162 | 30.355 | 98.038 | 172.646 | 1.00 | 24.23 |
| 1322 | CB | SER | A | 162 | 29.258 | 98.474 | 171.679 | 1.00 | 85.17 |
| 1323 | OG | SER | A | 162 | 29.691 | 98.382 | 170.325 | 1.00 | 33.98 |
| 1324 | C | SER | A | 162 | 30.546 | 96.549 | 172.518 | 1.00 | 41.42 |
| 1325 | O | SER | A | 162 | 31.379 | 96.108 | 171.739 | 1.00 | 40.06 |
| 1326 | N | GLU | A | 163 | 29.828 | 95.793 | 173.363 | 1.00 | 47.31 |
| 1327 | CA | GLU | A | 163 | 29.945 | 94.371 | 173.069 | 1.00 | 32.63 |
| 1328 | CB | GLU | A | 163 | 29.169 | 93.547 | 174.097 | 1.00 | 99.44 |
| 1329 | CG | GLU | A | 163 | 29.649 | 93.729 | 175.527 | 1.00 | 204.45 |
| 1330 | CD | GLU | A | 163 | 28.850 | 92.906 | 176.519 | 1.00 | 213.27 |
| 1331 | OE1 | GLU | A | 163 | 27.925 | 92.187 | 176.086 | 1.00 | 189.41 |
| 1332 | OE2 | GLU | A | 163 | 29.148 | 92.980 | 177.729 | 1.00 | 180.03 |
| 1333 | C | GLU | A | 163 | 29.462 | 94.059 | 171.657 | 1.00 | 38.80 |
| 1334 | O | GLU | A | 163 | 28.601 | 94.902 | 171.253 | 1.00 | 51.57 |
| 1335 | N | PRO | A | 164 | 30.006 | 93.173 | 170.942 | 1.00 | 52.67 |
| 1336 | CD | PRO | A | 164 | 30.968 | 92.184 | 171.457 | 1.00 | 85.33 |
| 1337 | CA | PRO | A | 164 | 29.650 | 92.869 | 169.551 | 1.00 | 18.17 |
| 1338 | CB | PRO | A | 164 | 30.698 | 91.847 | 169.156 | 1.00 | 48.35 |
| 1339 | CG | PRO | A | 164 | 30.886 | 91.089 | 170.416 | 1.00 | 83.53 |
| 1340 | C | PRO | A | 164 | 28.238 | 92.378 | 169.300 | 1.00 | 33.91 |
| 1341 | O | PRO | A | 164 | 27.605 | 91.799 | 170.180 | 1.00 | 41.04 |
| 1342 | N | LEU | A | 165 | 27.760 | 92.592 | 168.077 | 1.00 | 50.63 |
| 1343 | CA | LEU | A | 165 | 26.402 | 92.200 | 167.706 | 1.00 | 22.85 |
| 1344 | CB | LEU | A | 165 | 25.483 | 93.404 | 167.777 | 1.00 | 65.50 |
| 1345 | CG | LEU | A | 165 | 24.027 | 93.057 | 167.530 | 1.00 | 5.42 |
| 1346 | CD1 | LEU | A | 165 | 23.591 | 92.064 | 168.578 | 1.00 | 85.48 |
| 1347 | CD2 | LEU | A | 165 | 23.182 | 94.310 | 167.584 | 1.00 | 73.11 |
| 1348 | C | LEU | A | 165 | 26.254 | 91.591 | 166.323 | 1.00 | 58.98 |
| 1349 | O | LEU | A | 165 | 26.696 | 92.171 | 165.326 | 1.00 | 45.85 |
| 1350 | N | ASN | A | 166 | 25.586 | 90.441 | 166.269 | 1.00 | 60.58 |
| 1351 | CA | ASN | A | 166 | 25.362 | 89.731 | 165.018 | 1.00 | 45.14 |
| 1352 | CB | ASN | A | 166 | 25.362 | 88.226 | 165.258 | 1.00 | 79.77 |
| 1353 | CG | ASN | A | 166 | 26.748 | 87.681 | 165.468 | 1.00 | 84.12 |
| 1354 | OD1 | ASN | A | 166 | 27.672 | 88.071 | 164.756 | 1.00 | 37.27 |
| 1355 | ND2 | ASN | A | 166 | 26.900 | 86.775 | 166.428 | 1.00 | 83.10 |
| 1356 | C | ASN | A | 166 | 24.079 | 90.124 | 164.312 | 1.00 | 53.27 |
| 1357 | O | ASN | A | 166 | 23.018 | 90.241 | 164.919 | 1.00 | 65.38 |
| 1358 | N | ILE | A | 167 | 24.195 | 90.314 | 163.008 | 1.00 | 52.39 |
| 1359 | CA | ILE | A | 167 | 23.071 | 90.689 | 162.171 | 1.00 | 51.60 |
| 1360 | CB | ILE | A | 167 | 23.197 | 92.152 | 161.735 | 1.00 | 54.96 |
| 1361 | CG2 | ILE | A | 167 | 22.259 | 92.454 | 160.598 | 1.00 | 34.47 |
| 1362 | CG1 | ILE | A | 167 | 22.875 | 93.055 | 162.912 | 1.00 | 29.93 |
| 1363 | CD1 | ILE | A | 167 | 23.061 | 94.512 | 162.595 | 1.00 | 110.11 |
| 1364 | C | ILE | A | 167 | 23.023 | 89.784 | 160.944 | 1.00 | 77.50 |
| 1365 | O | ILE | A | 167 | 24.024 | 89.605 | 160.245 | 1.00 | 94.63 |
| 1366 | N | THR | A | 168 | 21.844 | 89.226 | 160.691 | 1.00 | 79.37 |
| 1367 | CA | THR | A | 168 | 21.635 | 88.318 | 159.573 | 1.00 | 66.36 |
| 1368 | CB | THR | A | 168 | 21.250 | 86.945 | 160.109 | 1.00 | 55.12 |
| 1369 | OG1 | THR | A | 168 | 20.149 | 87.094 | 161.014 | 1.00 | 103.78 |
| 1370 | CG2 | THR | A | 168 | 22.416 | 86.313 | 160.856 | 1.00 | 86.09 |
| 1371 | C | THR | A | 168 | 20.545 | 88.797 | 158.607 | 1.00 | 74.59 |
| 1372 | O | THR | A | 168 | 19.565 | 89.412 | 159.024 | 1.00 | 64.58 |
| 1373 | N | VAL | A | 169 | 20.718 | 88.502 | 157.320 | 1.00 | 76.13 |
| 1374 | CA | VAL | A | 169 | 19.752 | 88.894 | 156.291 | 1.00 | 70.39 |
| 1375 | CB | VAL | A | 169 | 20.320 | 90.056 | 155.456 | 1.00 | 62.07 |
| 1376 | CG1 | VAL | A | 169 | 19.381 | 90.419 | 154.335 | 1.00 | 100.15 |
| 1377 | CG2 | VAL | A | 169 | 20.529 | 91.251 | 156.341 | 1.00 | 62.65 |
| 1378 | C | VAL | A | 169 | 19.382 | 87.703 | 155.386 | 1.00 | 94.90 |
| 1379 | O | VAL | A | 169 | 20.259 | 86.968 | 154.931 | 1.00 | 115.53 |
| 1380 | N | ILE | A | 170 | 18.081 | 87.537 | 155.123 | 1.00 | 88.61 |
| 1381 | CA | ILE | A | 170 | 17.538 | 86.422 | 154.329 | 1.00 | 131.29 |
| 1382 | CB | ILE | A | 170 | 16.224 | 85.933 | 154.969 | 1.00 | 132.04 |
| 1383 | CG2 | ILE | A | 170 | 15.908 | 84.511 | 154.513 | 1.00 | 178.52 |
| 1384 | CG1 | ILE | A | 170 | 16.360 | 85.966 | 156.492 | 1.00 | 77.40 |
| 1385 | CD1 | ILE | A | 170 | 15.077 | 85.661 | 157.225 | 1.00 | 74.21 |
| 1386 | C | ILE | A | 170 | 17.264 | 86.673 | 152.835 | 1.00 | 163.13 |
| 1387 | O | ILE | A | 170 | 17.487 | 87.768 | 152.330 | 1.00 | 191.77 |
| 1388 | N | LYS | A | 171 | 16.781 | 85.637 | 152.142 | 1.00 | 174.37 |
| 1389 | CA | LYS | A | 171 | 16.446 | 85.699 | 150.713 | 1.00 | 184.81 |
| 1390 | CB | LYS | A | 171 | 17.572 | 85.098 | 149.861 | 1.00 | 169.16 |
| 1391 | CG | LYS | A | 171 | 18.821 | 85.950 | 149.771 | 1.00 | 171.69 |
| 1392 | CD | LYS | A | 171 | 19.841 | 85.352 | 148.814 | 1.00 | 165.73 |
| 1393 | CE | LYS | A | 171 | 21.059 | 86.259 | 148.692 | 1.00 | 166.52 |
| 1394 | NZ | LYS | A | 171 | 22.110 | 85.721 | 147.784 | 1.00 | 163.94 |
| 1395 | C | LYS | A | 171 | 15.152 | 84.928 | 150.430 | 1.00 | 199.02 |
| 1396 | O | LYS | A | 171 | 14.841 | 83.961 | 151.124 | 1.00 | 217.89 |
| 1397 | N | ALA | A | 172 | 14.405 | 85.349 | 149.410 | 1.00 | 194.74 |
| 1398 | CA | ALA | A | 172 | 13.152 | 84.681 | 149.047 | 1.00 | 171.94 |
| 1399 | CB | ALA | A | 172 | 11.960 | 85.501 | 149.535 | 1.00 | 113.16 |
| 1400 | C | ALA | A | 172 | 13.055 | 84.474 | 147.535 | 1.00 | 174.40 |
| 1401 | O | ALA | A | 172 | 12.810 | 85.420 | 146.788 | 1.00 | 182.27 |
| 1402 | N | PRO | A | 173 | 13.241 | 83.227 | 147.067 | 1.00 | 195.13 |
| 1403 | CD | PRO | A | 173 | 13.548 | 82.010 | 147.842 | 1.00 | 150.42 |
| 1404 | CA | PRO | A | 173 | 13.170 | 82.925 | 145.632 | 1.00 | 196.30 |
| 1405 | CB | PRO | A | 173 | 13.702 | 81.495 | 145.558 | 1.00 | 174.85 |
| 1406 | CG | PRO | A | 173 | 13.235 | 80.907 | 146.852 | 1.00 | 159.72 |
| 1407 | C | PRO | A | 173 | 11.765 | 83.064 | 145.034 | 1.00 | 184.97 |
| 1408 | O | PRO | A | 173 | 11.238 | 82.054 | 144.522 | 1.00 | 174.19 |
| 1409 | OXT | PRO | A | 173 | 11.213 | 84.183 | 145.089 | 1.00 | 91.11 |
| 1410 | C1 | NAG | A | 221 | 11.009 | 106.713 | 181.607 | 1.00 | 78.57 |
| 1411 | C2 | NAG | A | 221 | 11.997 | 107.878 | 181.655 | 1.00 | 100.89 |
| 1412 | N2 | NAG | A | 221 | 13.311 | 107.471 | 181.201 | 1.00 | 76.21 |
| 1413 | C7 | NAG | A | 221 | 13.976 | 108.256 | 180.361 | 1.00 | 117.38 |
| 1414 | O7 | NAG | A | 221 | 13.803 | 108.231 | 179.142 | 1.00 | 148.10 |
| 1415 | C8 | NAG | A | 221 | 14.971 | 109.233 | 180.966 | 1.00 | 135.41 |
| 1416 | C3 | NAG | A | 221 | 12.062 | 108.405 | 183.087 | 1.00 | 127.09 |
| 1417 | O3 | NAG | A | 221 | 12.916 | 109.541 | 183.151 | 1.00 | 152.40 |
| 1418 | C4 | NAG | A | 221 | 10.653 | 108.784 | 183.562 | 1.00 | 126.14 |
| 1419 | O4 | NAG | A | 221 | 10.688 | 109.080 | 184.970 | 1.00 | 148.94 |
| 1420 | C5 | NAG | A | 221 | 9.653 | 107.645 | 183.321 | 1.00 | 133.04 |
| 1421 | O5 | NAG | A | 221 | 9.707 | 107.189 | 181.953 | 1.00 | 70.49 |
| 1422 | C6 | NAG | A | 221 | 8.220 | 108.040 | 183.621 | 1.00 | 149.52 |
| 1423 | O6 | NAG | A | 221 | 7.694 | 108.868 | 182.567 | 1.00 | 151.09 |
| 1424 | C1 | NAG | A | 222 | 10.235 | 110.337 | 185.310 | 1.00 | 150.76 |
| 1425 | C2 | NAG | A | 222 | 9.719 | 110.337 | 186.759 | 1.00 | 157.88 |
| 1426 | N2 | NAG | A | 222 | 8.580 | 109.445 | 186.884 | 1.00 | 152.33 |
| 1427 | C7 | NAG | A | 222 | 8.427 | 108.704 | 187.977 | 1.00 | 149.05 |
| 1428 | O7 | NAG | A | 222 | 9.078 | 107.677 | 188.190 | 1.00 | 108.16 |
| 1429 | C8 | NAG | A | 222 | 7.395 | 109.174 | 188.991 | 1.00 | 148.72 |
| 1430 | C3 | NAG | A | 222 | 9.316 | 111.762 | 187.139 | 1.00 | 163.52 |
| 1431 | O3 | NAG | A | 222 | 8.887 | 111.804 | 188.493 | 1.00 | 159.10 |
| 1432 | C4 | NAG | A | 222 | 10.521 | 112.683 | 186.929 | 1.00 | 168.36 |
| 1433 | O4 | NAG | A | 222 | 10.184 | 114.036 | 187.276 | 1.00 | 195.32 |
| 1434 | C5 | NAG | A | 222 | 10.970 | 112.600 | 185.466 | 1.00 | 142.19 |
| 1435 | O5 | NAG | A | 222 | 11.333 | 111.244 | 185.156 | 1.00 | 131.31 |
| 1436 | C6 | NAG | A | 222 | 12.167 | 113.462 | 185.127 | 1.00 | 147.99 |
| 1437 | O6 | NAG | A | 222 | 12.730 | 113.081 | 183.879 | 1.00 | 139.76 |
| 1438 | C1 | MAN | A | 223 | 10.805 | 114.503 | 188.420 | 1.00 | 185.67 |
| 1439 | C2 | MAN | A | 223 | 10.910 | 116.025 | 188.373 | 1.00 | 183.28 |
| 1440 | O2 | MAN | A | 223 | 9.623 | 116.596 | 188.179 | 1.00 | 182.05 |
| 1441 | C3 | MAN | A | 223 | 11.524 | 116.542 | 189.677 | 1.00 | 206.08 |
| 1442 | O3 | MAN | A | 223 | 11.463 | 117.961 | 189.691 | 1.00 | 200.83 |
| 1443 | C4 | MAN | A | 223 | 10.787 | 115.976 | 190.907 | 1.00 | 228.59 |
| 1444 | O4 | MAN | A | 223 | 11.500 | 116.305 | 192.093 | 1.00 | 213.28 |
| 1445 | C5 | MAN | A | 223 | 10.646 | 114.450 | 190.805 | 1.00 | 211.66 |
| 1446 | O5 | MAN | A | 223 | 10.026 | 114.090 | 189.551 | 1.00 | 206.15 |
| 1447 | C6 | MAN | A | 223 | 9.793 | 113.860 | 191.919 | 1.00 | 191.52 |
| 1448 | O6 | MAN | A | 223 | 8.598 | 113.277 | 191.412 | 1.00 | 161.92 |
| 1449 | C1 | FUC | A | 224 | 7.359 | 110.170 | 182.978 | 1.00 | 147.50 |
| 1450 | C2 | FUC | A | 224 | 6.361 | 110.756 | 181.982 | 1.00 | 163.07 |
| 1451 | O2 | FUC | A | 224 | 6.901 | 110.689 | 180.670 | 1.00 | 142.50 |
| 1452 | C3 | FUC | A | 224 | 5.059 | 109.950 | 182.059 | 1.00 | 147.28 |
| 1453 | O3 | FUC | A | 224 | 4.101 | 110.474 | 181.149 | 1.00 | 122.66 |
| 1454 | C4 | FUC | A | 224 | 4.509 | 109.992 | 183.492 | 1.00 | 181.93 |
| 1455 | O4 | FUC | A | 224 | 4.136 | 111.323 | 183.829 | 1.00 | 194.01 |
| 1456 | C5 | FUC | A | 224 | 5.573 | 109.491 | 184.480 | 1.00 | 172.04 |
| 1457 | O5 | FUC | A | 224 | 6.810 | 110.224 | 184.301 | 1.00 | 162.84 |
| 1458 | C6 | FUC | A | 224 | 5.158 | 109.661 | 185.932 | 1.00 | 120.33 |
| 1459 | C1 | NAG | A | 242 | 13.815 | 85.747 | 181.704 | 1.00 | 57.19 |
| 1460 | C2 | NAG | A | 242 | 13.676 | 86.167 | 183.149 | 1.00 | 45.95 |
| 1461 | N2 | NAG | A | 242 | 12.332 | 86.630 | 183.415 | 1.00 | 15.33 |
| 1462 | C7 | NAG | A | 242 | 11.817 | 86.468 | 184.631 | 1.00 | 112.91 |
| 1463 | O7 | NAG | A | 242 | 11.620 | 85.356 | 185.127 | 1.00 | 100.48 |
| 1464 | C8 | NAG | A | 242 | 11.482 | 87.721 | 185.427 | 1.00 | 16.99 |
| 1465 | C3 | NAG | A | 242 | 14.703 | 87.257 | 183.411 | 1.00 | 60.32 |
| 1466 | O3 | NAG | A | 242 | 14.623 | 87.699 | 184.764 | 1.00 | 42.86 |
| 1467 | C4 | NAG | A | 242 | 16.114 | 86.722 | 183.109 | 1.00 | 61.08 |
| 1468 | O4 | NAG | A | 242 | 17.042 | 87.825 | 183.150 | 1.00 | 83.57 |
| 1469 | C5 | NAG | A | 242 | 16.181 | 86.065 | 181.715 | 1.00 | 41.47 |
| 1470 | O5 | NAG | A | 242 | 15.096 | 85.134 | 181.520 | 1.00 | 54.87 |
| 1471 | C6 | NAG | A | 242 | 17.467 | 85.288 | 181.499 | 1.00 | 119.52 |
| 1472 | O6 | NAG | A | 242 | 17.381 | 83.969 | 182.022 | 1.00 | 140.01 |
| 1473 | C1 | NAG | A | 243 | 18.183 | 87.704 | 183.928 | 1.00 | 81.48 |
| 1474 | C2 | NAG | A | 243 | 19.362 | 88.235 | 183.120 | 1.00 | 38.25 |
| 1475 | N2 | NAG | A | 243 | 19.591 | 87.363 | 181.993 | 1.00 | 69.21 |
| 1476 | C7 | NAG | A | 243 | 19.577 | 87.863 | 180.768 | 1.00 | 66.57 |
| 1477 | O7 | NAG | A | 243 | 19.393 | 89.057 | 180.540 | 1.00 | 94.86 |
| 1478 | C8 | NAG | A | 243 | 19.805 | 86.892 | 179.623 | 1.00 | 48.41 |
| 1479 | C3 | NAG | A | 243 | 20.625 | 88.312 | 183.964 | 1.00 | 87.22 |
| 1480 | O3 | NAG | A | 243 | 21.674 | 88.894 | 183.208 | 1.00 | 98.62 |
| 1481 | C4 | NAG | A | 243 | 20.364 | 89.143 | 185.212 | 1.00 | 76.30 |
| 1482 | O4 | NAG | A | 243 | 21.549 | 89.126 | 186.040 | 1.00 | 94.39 |
| 1483 | C5 | NAG | A | 243 | 19.170 | 88.509 | 185.959 | 1.00 | 102.39 |
| 1484 | O5 | NAG | A | 243 | 17.998 | 88.494 | 185.115 | 1.00 | 49.63 |
| 1485 | C6 | NAG | A | 243 | 18.782 | 89.210 | 187.244 | 1.00 | 122.15 |
| 1486 | O6 | NAG | A | 243 | 17.997 | 88.358 | 188.067 | 1.00 | 105.32 |
| 1487 | C1 | MAN | A | 244 | 22.078 | 90.350 | 186.412 | 1.00 | 64.85 |
| 1488 | C2 | MAN | A | 244 | 22.728 | 90.214 | 187.783 | 1.00 | 116.22 |
| 1489 | O2 | MAN | A | 244 | 23.684 | 89.161 | 187.744 | 1.00 | 77.96 |
| 1490 | C3 | MAN | A | 244 | 23.402 | 91.540 | 188.186 | 1.00 | 112.21 |
| 1491 | O3 | MAN | A | 244 | 24.150 | 91.370 | 189.413 | 1.00 | 152.31 |
| 1492 | C4 | MAN | A | 244 | 24.351 | 92.024 | 187.075 | 1.00 | 144.57 |
| 1493 | O4 | MAN | A | 244 | 24.813 | 93.333 | 187.385 | 1.00 | 193.98 |
| 1494 | C5 | MAN | A | 244 | 23.633 | 92.031 | 185.713 | 1.00 | 57.60 |
| 1495 | O5 | MAN | A | 244 | 23.067 | 90.728 | 185.441 | 1.00 | 80.63 |
| 1496 | C6 | MAN | A | 244 | 24.504 | 92.436 | 184.513 | 1.00 | 53.28 |
| 1497 | O6 | MAN | A | 244 | 25.641 | 91.560 | 184.352 | 1.00 | 64.48 |
| 1498 | C1 | MAN | A | 245 | 23.427 | 91.459 | 190.614 | 1.00 | 134.73 |
| 1499 | C2 | MAN | A | 245 | 24.400 | 91.435 | 191.803 | 1.00 | 145.85 |
| 1500 | O2 | MAN | A | 245 | 23.715 | 91.778 | 193.000 | 1.00 | 115.81 |
| 1501 | C3 | MAN | A | 245 | 25.063 | 90.050 | 191.951 | 1.00 | 134.74 |
| 1502 | O3 | MAN | A | 245 | 25.754 | 89.986 | 193.192 | 1.00 | 105.27 |
| 1503 | C4 | MAN | A | 245 | 24.043 | 88.898 | 191.885 | 1.00 | 133.62 |
| 1504 | O4 | MAN | A | 245 | 24.736 | 87.669 | 191.714 | 1.00 | 67.76 |
| 1505 | C5 | MAN | A | 245 | 23.061 | 89.079 | 190.719 | 1.00 | 165.40 |
| 1506 | O5 | MAN | A | 245 | 22.479 | 90.403 | 190.751 | 1.00 | 164.09 |
| 1507 | C6 | MAN | A | 245 | 21.918 | 88.081 | 190.747 | 1.00 | 136.99 |
| 1508 | O6 | MAN | A | 245 | 20.800 | 88.600 | 191.453 | 1.00 | 163.94 |
| 1509 | C1 | MAN | A | 246 | 26.745 | 92.247 | 183.813 | 1.00 | 91.80 |
| 1510 | C2 | MAN | A | 246 | 27.492 | 91.359 | 182.813 | 1.00 | 89.53 |
| 1511 | O2 | MAN | A | 246 | 28.434 | 92.147 | 182.107 | 1.00 | 75.32 |
| 1512 | C3 | MAN | A | 246 | 28.223 | 90.227 | 183.536 | 1.00 | 97.98 |
| 1513 | O3 | MAN | A | 246 | 28.995 | 89.485 | 182.603 | 1.00 | 123.68 |
| 1514 | C4 | MAN | A | 246 | 29.139 | 90.790 | 184.628 | 1.00 | 99.73 |
| 1515 | O4 | MAN | A | 246 | 29.701 | 89.712 | 185.368 | 1.00 | 70.44 |
| 1516 | C5 | MAN | A | 246 | 28.338 | 91.709 | 185.566 | 1.00 | 111.67 |
| 1517 | O5 | MAN | A | 246 | 27.651 | 92.738 | 184.808 | 1.00 | 73.91 |
| 1518 | C6 | MAN | A | 246 | 29.187 | 92.408 | 186.620 | 1.00 | 133.93 |
| 1519 | O6 | MAN | A | 246 | 30.118 | 93.314 | 186.037 | 1.00 | 157.23 |
| 1520 | C1 | NAG | A | 366 | 28.056 | 85.901 | 166.422 | 1.00 | 118.02 |
| 1521 | C2 | NAG | A | 366 | 27.711 | 84.597 | 167.109 | 1.00 | 144.13 |
| 1522 | N2 | NAG | A | 366 | 27.168 | 84.844 | 168.429 | 1.00 | 145.90 |
| 1523 | C7 | NAG | A | 366 | 26.706 | 83.827 | 169.147 | 1.00 | 187.35 |
| 1524 | O7 | NAG | A | 366 | 27.404 | 83.211 | 169.952 | 1.00 | 194.87 |
| 1525 | C8 | NAG | A | 366 | 25.255 | 83.418 | 168.931 | 1.00 | 170.13 |
| 1526 | C3 | NAG | A | 366 | 28.966 | 83.736 | 167.196 | 1.00 | 142.04 |
| 1527 | O3 | NAG | A | 366 | 28.630 | 82.485 | 167.776 | 1.00 | 194.55 |
| 1528 | C4 | NAG | A | 366 | 29.556 | 83.514 | 165.790 | 1.00 | 143.64 |
| 1529 | O4 | NAG | A | 366 | 30.849 | 82.871 | 165.890 | 1.00 | 198.08 |
| 1530 | C5 | NAG | A | 366 | 29.712 | 84.852 | 165.035 | 1.00 | 84.06 |
| 1531 | O5 | NAG | A | 366 | 28.487 | 85.621 | 165.083 | 1.00 | 133.37 |
| 1532 | C6 | NAG | A | 366 | 30.057 | 84.664 | 163.560 | 1.00 | 113.91 |
| 1533 | O6 | NAG | A | 366 | 29.035 | 83.880 | 162.905 | 1.00 | 159.48 |
| 1534 | C1 | NAG | A | 367 | 30.856 | 81.509 | 166.161 | 1.00 | 189.45 |
| 1535 | C2 | NAG | A | 367 | 32.125 | 80.858 | 165.606 | 1.00 | 164.69 |
| 1536 | N2 | NAG | A | 367 | 32.162 | 81.012 | 164.163 | 1.00 | 194.38 |
| 1537 | C7 | NAG | A | 367 | 33.110 | 81.749 | 163.590 | 1.00 | 201.09 |
| 1538 | O7 | NAG | A | 367 | 33.517 | 82.807 | 164.072 | 1.00 | 175.40 |
| 1539 | C8 | NAG | A | 367 | 33.703 | 81.229 | 162.288 | 1.00 | 186.30 |
| 1540 | C3 | NAG | A | 367 | 32.134 | 79.368 | 165.981 | 1.00 | 171.43 |
| 1541 | O3 | NAG | A | 367 | 33.372 | 78.785 | 165.603 | 1.00 | 182.11 |
| 1542 | C4 | NAG | A | 367 | 31.925 | 79.180 | 167.489 | 1.00 | 181.87 |
| 1543 | O4 | NAG | A | 367 | 31.768 | 77.799 | 167.780 | 1.00 | 183.58 |
| 1544 | C5 | NAG | A | 367 | 30.683 | 79.946 | 167.949 | 1.00 | 186.74 |
| 1545 | O5 | NAG | A | 367 | 30.802 | 81.334 | 167.581 | 1.00 | 198.91 |
| 1546 | C6 | NAG | A | 367 | 30.463 | 79.898 | 169.448 | 1.00 | 186.99 |
| 1547 | O6 | NAG | A | 367 | 29.081 | 79.771 | 169.756 | 1.00 | 172.34 |
| 1548 | C1 | FUC | A | 369 | 29.475 | 83.367 | 161.677 | 1.00 | 178.37 |
| 1549 | C2 | FUC | A | 369 | 28.873 | 81.974 | 161.447 | 1.00 | 178.90 |
| 1550 | O2 | FUC | A | 369 | 29.095 | 81.158 | 162.587 | 1.00 | 137.53 |
| 1551 | C3 | FUC | A | 369 | 27.373 | 82.084 | 161.176 | 1.00 | 178.26 |
| 1552 | O3 | FUC | A | 369 | 26.837 | 80.797 | 160.906 | 1.00 | 126.18 |
| 1553 | C4 | FUC | A | 369 | 27.145 | 83.010 | 159.982 | 1.00 | 193.60 |
| 1554 | O4 | FUC | A | 369 | 27.752 | 82.452 | 158.825 | 1.00 | 190.82 |
| 1555 | C5 | FUC | A | 369 | 27.765 | 84.381 | 160.283 | 1.00 | 182.89 |
| 1556 | O5 | FUC | A | 369 | 29.175 | 84.233 | 160.576 | 1.00 | 193.18 |
| 1557 | C6 | FUC | A | 369 | 27.641 | 85.357 | 159.126 | 1.00 | 137.51 |
| 1558 | CB | PRO | B | 328 | 44.233 | 128.245 | 175.766 | 1.00 | 170.67 |
| 1559 | CG | PRO | B | 328 | 43.202 | 128.349 | 176.889 | 1.00 | 177.05 |
| 1560 | C | PRO | B | 328 | 43.060 | 126.964 | 173.946 | 1.00 | 208.41 |
| 1561 | O | PRO | B | 328 | 43.981 | 126.261 | 173.518 | 1.00 | 173.87 |
| 1562 | N | PRO | B | 328 | 42.116 | 129.063 | 174.936 | 1.00 | 199.93 |
| 1563 | CD | PRO | B | 328 | 42.170 | 129.366 | 176.377 | 1.00 | 189.57 |
| 1564 | CA | PRO | B | 328 | 43.348 | 128.347 | 174.529 | 1.00 | 198.40 |
| 1565 | N | CYS | B | 329 | 41.785 | 126.575 | 173.931 | 1.00 | 223.49 |
| 1566 | CA | CYS | B | 329 | 41.399 | 125.277 | 173.386 | 1.00 | 213.48 |
| 1567 | C | CYS | B | 329 | 40.595 | 125.390 | 172.094 | 1.00 | 210.28 |
| 1568 | O | CYS | B | 329 | 39.925 | 124.441 | 171.686 | 1.00 | 203.81 |
| 1569 | CB | CYS | B | 329 | 40.596 | 124.462 | 174.402 | 1.00 | 202.41 |
| 1570 | SG | CYS | B | 329 | 40.352 | 122.737 | 173.856 | 1.00 | 228.51 |
| 1571 | N | ASP | B | 330 | 40.647 | 126.557 | 171.461 | 1.00 | 211.89 |
| 1572 | CA | ASP | B | 330 | 39.960 | 126.766 | 170.189 | 1.00 | 192.96 |
| 1573 | CB | ASP | B | 330 | 39.714 | 128.266 | 169.964 | 1.00 | 206.60 |
| 1574 | CG | ASP | B | 330 | 38.919 | 128.560 | 168.691 | 1.00 | 207.78 |
| 1575 | OD1 | ASP | B | 330 | 39.428 | 128.310 | 167.577 | 1.00 | 198.18 |
| 1576 | OD2 | ASP | B | 330 | 37.778 | 129.052 | 168.806 | 1.00 | 197.14 |
| 1577 | C | ASP | B | 330 | 40.978 | 126.228 | 169.184 | 1.00 | 177.46 |
| 1578 | O | ASP | B | 330 | 41.198 | 126.802 | 168.117 | 1.00 | 190.87 |
| 1579 | N | SER | B | 331 | 41.601 | 125.110 | 169.550 | 1.00 | 151.57 |
| 1580 | CA | SER | B | 331 | 42.636 | 124.492 | 168.731 | 1.00 | 109.39 |
| 1581 | CB | SER | B | 331 | 43.959 | 124.493 | 169.509 | 1.00 | 109.57 |
| 1582 | OG | SER | B | 331 | 43.867 | 123.728 | 170.706 | 1.00 | 75.56 |
| 1583 | C | SER | B | 331 | 42.353 | 123.073 | 168.245 | 1.00 | 79.09 |
| 1584 | O | SER | B | 331 | 43.245 | 122.411 | 167.724 | 1.00 | 130.44 |
| 1585 | N | ASN | B | 332 | 41.129 | 122.596 | 168.411 | 1.00 | 76.72 |
| 1586 | CA | ASN | B | 332 | 40.796 | 121.246 | 167.977 | 1.00 | 39.06 |
| 1587 | CB | ASN | B | 332 | 40.842 | 120.285 | 169.162 | 1.00 | 93.86 |
| 1588 | CG | ASN | B | 332 | 42.247 | 119.821 | 169.484 | 1.00 | 76.70 |
| 1589 | OD1 | ASN | B | 332 | 43.185 | 120.617 | 169.523 | 1.00 | 81.95 |
| 1590 | ND2 | ASN | B | 332 | 42.398 | 118.525 | 169.735 | 1.00 | 60.32 |
| 1591 | C | ASN | B | 332 | 39.424 | 121.189 | 167.336 | 1.00 | 50.77 |
| 1592 | O | ASN | B | 332 | 38.588 | 120.357 | 167.696 | 1.00 | 48.39 |
| 1593 | N | PRO | B | 333 | 39.173 | 122.079 | 166.373 | 1.00 | 10.22 |
| 1594 | CD | PRO | B | 333 | 40.098 | 123.063 | 165.794 | 1.00 | 26.58 |
| 1595 | CA | PRO | B | 333 | 37.889 | 122.113 | 165.689 | 1.00 | 43.44 |
| 1596 | CB | PRO | B | 333 | 38.232 | 122.764 | 164.362 | 1.00 | 11.09 |
| 1597 | CG | PRO | B | 333 | 39.219 | 123.787 | 164.780 | 1.00 | 73.63 |
| 1598 | C | PRO | B | 333 | 37.271 | 120.738 | 165.530 | 1.00 | 11.84 |
| 1599 | O | PRO | B | 333 | 37.932 | 119.767 | 165.206 | 1.00 | 62.32 |
| 1600 | N | ARG | B | 334 | 35.984 | 120.677 | 165.788 | 1.00 | 33.64 |
| 1601 | CA | ARG | B | 334 | 35.235 | 119.460 | 165.677 | 1.00 | 33.31 |
| 1602 | CB | ARG | B | 334 | 34.754 | 119.322 | 164.242 | 1.00 | 10.21 |
| 1603 | CG | ARG | B | 334 | 33.904 | 118.112 | 164.022 | 1.00 | 52.28 |
| 1604 | CD | ARG | B | 334 | 32.439 | 118.403 | 163.975 | 1.00 | 10.85 |
| 1605 | NE | ARG | B | 334 | 31.983 | 118.450 | 162.597 | 1.00 | 39.50 |
| 1606 | CZ | ARG | B | 334 | 30.763 | 118.103 | 162.210 | 1.00 | 75.73 |
| 1607 | NH1 | ARG | B | 334 | 29.885 | 117.676 | 163.099 | 1.00 | 59.45 |
| 1608 | NH2 | ARG | B | 334 | 30.416 | 118.202 | 160.936 | 1.00 | 61.90 |
| 1609 | C | ARG | B | 334 | 36.002 | 118.225 | 166.156 | 1.00 | 35.54 |
| 1610 | O | ARG | B | 334 | 35.615 | 117.090 | 165.891 | 1.00 | 57.63 |
| 1611 | N | GLY | B | 335 | 37.081 | 118.441 | 166.896 | 1.00 | 21.32 |
| 1612 | CA | GLY | B | 335 | 37.832 | 117.308 | 167.413 | 1.00 | 60.66 |
| 1613 | C | GLY | B | 335 | 39.060 | 116.972 | 166.600 | 1.00 | 46.23 |
| 1614 | O | GLY | B | 335 | 39.953 | 116.256 | 167.053 | 1.00 | 28.43 |
| 1615 | N | VAL | B | 336 | 39.087 | 117.491 | 165.381 | 1.00 | 51.37 |
| 1616 | CA | VAL | B | 336 | 40.196 | 117.306 | 164.454 | 1.00 | 36.83 |
| 1617 | CB | VAL | B | 336 | 39.836 | 117.952 | 163.124 | 1.00 | 62.63 |
| 1618 | CG1 | VAL | B | 336 | 41.025 | 117.972 | 162.204 | 1.00 | 75.33 |
| 1619 | CG2 | VAL | B | 336 | 38.674 | 117.213 | 162.512 | 1.00 | 96.26 |
| 1620 | C | VAL | B | 336 | 41.485 | 117.947 | 164.969 | 1.00 | 35.62 |
| 1621 | O | VAL | B | 336 | 41.596 | 119.164 | 164.967 | 1.00 | 59.08 |
| 1622 | N | SER | B | 337 | 42.456 | 117.148 | 165.404 | 1.00 | 44.91 |
| 1623 | CA | SER | B | 337 | 43.714 | 117.712 | 165.900 | 1.00 | 35.01 |
| 1624 | CB | SER | B | 337 | 44.232 | 116.895 | 167.081 | 1.00 | 70.32 |
| 1625 | OG | SER | B | 337 | 44.222 | 115.512 | 166.795 | 1.00 | 72.41 |
| 1626 | C | SER | B | 337 | 44.791 | 117.806 | 164.809 | 1.00 | 48.70 |
| 1627 | O | SER | B | 337 | 44.673 | 117.177 | 163.760 | 1.00 | 25.48 |
| 1628 | N | ALA | B | 338 | 45.827 | 118.612 | 165.058 | 1.00 | 48.51 |
| 1629 | CA | ALA | B | 338 | 46.939 | 118.821 | 164.107 | 1.00 | 82.57 |
| 1630 | CB | ALA | B | 338 | 46.705 | 120.063 | 163.245 | 1.00 | 7.76 |
| 1631 | C | ALA | B | 338 | 48.260 | 118.980 | 164.842 | 1.00 | 29.51 |
| 1632 | O | ALA | B | 338 | 48.301 | 119.477 | 165.968 | 1.00 | 84.58 |
| 1633 | N | TYR | B | 339 | 49.340 | 118.555 | 164.206 | 1.00 | 63.43 |
| 1634 | CA | TYR | B | 339 | 50.643 | 118.655 | 164.823 | 1.00 | 60.91 |
| 1635 | CB | TYR | B | 339 | 51.122 | 117.269 | 165.235 | 1.00 | 46.03 |
| 1636 | CG | TYR | B | 339 | 50.150 | 116.534 | 166.132 | 1.00 | 43.69 |
| 1637 | CD1 | TYR | B | 339 | 49.014 | 115.921 | 165.614 | 1.00 | 74.02 |
| 1638 | CE1 | TYR | B | 339 | 48.098 | 115.288 | 166.440 | 1.00 | 48.18 |
| 1639 | CD2 | TYR | B | 339 | 50.347 | 116.492 | 167.503 | 1.00 | 70.23 |
| 1640 | CE2 | TYR | B | 339 | 49.435 | 115.867 | 168.340 | 1.00 | 97.07 |
| 1641 | CZ | TYR | B | 339 | 48.310 | 115.270 | 167.804 | 1.00 | 86.05 |
| 1642 | OH | TYR | B | 339 | 47.383 | 114.696 | 168.648 | 1.00 | 102.25 |
| 1643 | C | TYR | B | 339 | 51.610 | 119.290 | 163.841 | 1.00 | 88.66 |
| 1644 | O | TYR | B | 339 | 51.530 | 119.048 | 162.640 | 1.00 | 48.34 |
| 1645 | N | LEU | B | 340 | 52.515 | 120.118 | 164.348 | 1.00 | 69.25 |
| 1646 | CA | LEU | B | 340 | 53.476 | 120.776 | 163.479 | 1.00 | 62.80 |
| 1647 | CB | LEU | B | 340 | 53.186 | 122.263 | 163.438 | 1.00 | 27.87 |
| 1648 | CG | LEU | B | 340 | 54.027 | 123.160 | 162.544 | 1.00 | 34.78 |
| 1649 | CD1 | LEU | B | 340 | 53.820 | 122.802 | 161.089 | 1.00 | 68.67 |
| 1650 | CD2 | LEU | B | 340 | 53.615 | 124.594 | 162.770 | 1.00 | 83.17 |
| 1651 | C | LEU | B | 340 | 54.881 | 120.531 | 163.988 | 1.00 | 79.88 |
| 1652 | O | LEU | B | 340 | 55.294 | 121.133 | 164.972 | 1.00 | 77.02 |
| 1653 | N | SER | B | 341 | 55.612 | 119.651 | 163.305 | 1.00 | 82.92 |
| 1654 | CA | SER | B | 341 | 56.964 | 119.290 | 163.712 | 1.00 | 91.09 |
| 1655 | CB | SER | B | 341 | 57.333 | 117.925 | 163.139 | 1.00 | 105.90 |
| 1656 | OG | SER | B | 341 | 58.517 | 117.431 | 163.744 | 1.00 | 156.83 |
| 1657 | C | SER | B | 341 | 58.023 | 120.306 | 163.321 | 1.00 | 86.74 |
| 1658 | O | SER | B | 341 | 57.918 | 120.956 | 162.286 | 1.00 | 74.60 |
| 1659 | N | ARG | B | 342 | 59.045 | 120.419 | 164.168 | 1.00 | 108.27 |
| 1660 | CA | ARG | B | 342 | 60.165 | 121.343 | 163.976 | 1.00 | 58.14 |
| 1661 | CB | ARG | B | 342 | 60.602 | 121.890 | 165.343 | 1.00 | 98.53 |
| 1662 | CG | ARG | B | 342 | 60.649 | 120.808 | 166.429 | 1.00 | 141.70 |
| 1663 | CD | ARG | B | 342 | 61.354 | 121.239 | 167.726 | 1.00 | 145.30 |
| 1664 | NE | ARG | B | 342 | 60.568 | 122.139 | 168.567 | 1.00 | 104.32 |
| 1665 | CZ | ARG | B | 342 | 60.430 | 123.442 | 168.351 | 1.00 | 124.08 |
| 1666 | NH1 | ARG | B | 342 | 61.028 | 124.015 | 167.315 | 1.00 | 83.45 |
| 1667 | NH2 | ARG | B | 342 | 59.691 | 124.174 | 169.173 | 1.00 | 139.46 |
| 1668 | C | ARG | B | 342 | 61.352 | 120.635 | 163.287 | 1.00 | 98.70 |
| 1669 | O | ARG | B | 342 | 61.582 | 119.441 | 163.500 | 1.00 | 94.69 |
| 1670 | N | PRO | B | 343 | 62.120 | 121.368 | 162.457 | 1.00 | 62.71 |
| 1671 | CD | PRO | B | 343 | 62.016 | 122.823 | 162.278 | 1.00 | 69.22 |
| 1672 | CA | PRO | B | 343 | 63.281 | 120.850 | 161.728 | 1.00 | 36.63 |
| 1673 | CB | PRO | B | 343 | 63.916 | 122.104 | 161.148 | 1.00 | 63.75 |
| 1674 | CG | PRO | B | 343 | 62.776 | 123.035 | 161.003 | 1.00 | 46.63 |
| 1675 | C | PRO | B | 343 | 64.234 | 120.174 | 162.689 | 1.00 | 50.31 |
| 1676 | O | PRO | B | 343 | 64.518 | 120.713 | 163.762 | 1.00 | 73.12 |
| 1677 | N | SER | B | 344 | 64.737 | 119.002 | 162.311 | 1.00 | 83.43 |
| 1678 | CA | SER | B | 344 | 65.671 | 118.289 | 163.177 | 1.00 | 70.61 |
| 1679 | CB | SER | B | 344 | 65.778 | 116.812 | 162.781 | 1.00 | 82.96 |
| 1680 | OG | SER | B | 344 | 66.324 | 116.661 | 161.483 | 1.00 | 84.14 |
| 1681 | C | SER | B | 344 | 67.024 | 118.946 | 163.041 | 1.00 | 65.68 |
| 1682 | O | SER | B | 344 | 67.334 | 119.517 | 161.998 | 1.00 | 50.07 |
| 1683 | N | PRO | B | 345 | 67.844 | 118.894 | 164.104 | 1.00 | 74.48 |
| 1684 | CD | PRO | B | 345 | 67.611 | 118.184 | 165.375 | 1.00 | 88.87 |
| 1685 | CA | PRO | B | 345 | 69.183 | 119.494 | 164.081 | 1.00 | 69.58 |
| 1686 | CB | PRO | B | 345 | 69.862 | 118.867 | 165.296 | 1.00 | 81.66 |
| 1687 | CG | PRO | B | 345 | 68.745 | 118.694 | 166.255 | 1.00 | 59.69 |
| 1688 | C | PRO | B | 345 | 69.849 | 119.065 | 162.789 | 1.00 | 83.15 |
| 1689 | O | PRO | B | 345 | 70.233 | 119.893 | 161.960 | 1.00 | 54.26 |
| 1690 | N | PHE | B | 346 | 69.955 | 117.745 | 162.642 | 1.00 | 65.25 |
| 1691 | CA | PHE | B | 346 | 70.542 | 117.099 | 161.482 | 1.00 | 38.65 |
| 1692 | CB | PHE | B | 346 | 70.209 | 115.611 | 161.522 | 1.00 | 67.61 |
| 1693 | CG | PHE | B | 346 | 70.755 | 114.839 | 160.365 | 1.00 | 96.31 |
| 1694 | CD1 | PHE | B | 346 | 72.119 | 114.727 | 160.181 | 1.00 | 76.06 |
| 1695 | CD2 | PHE | B | 346 | 69.903 | 114.239 | 159.445 | 1.00 | 132.61 |
| 1696 | CE1 | PHE | B | 346 | 72.632 | 114.039 | 159.104 | 1.00 | 96.72 |
| 1697 | CE2 | PHE | B | 346 | 70.410 | 113.544 | 158.358 | 1.00 | 124.94 |
| 1698 | CZ | PHE | B | 346 | 71.779 | 113.445 | 158.187 | 1.00 | 134.65 |
| 1699 | C | PHE | B | 346 | 70.045 | 117.721 | 160.170 | 1.00 | 58.29 |
| 1700 | O | PHE | B | 346 | 70.796 | 118.379 | 159.474 | 1.00 | 57.14 |
| 1701 | N | ASP | B | 347 | 68.777 | 117.524 | 159.831 | 1.00 | 56.88 |
| 1702 | CA | ASP | B | 347 | 68.226 | 118.078 | 158.592 | 1.00 | 64.19 |
| 1703 | CB | ASP | B | 347 | 66.703 | 117.859 | 158.542 | 1.00 | 98.15 |
| 1704 | CG | ASP | B | 347 | 66.318 | 116.431 | 158.211 | 1.00 | 120.11 |
| 1705 | OD1 | ASP | B | 347 | 66.506 | 116.024 | 157.046 | 1.00 | 142.93 |
| 1706 | OD2 | ASP | B | 347 | 65.826 | 115.715 | 159.112 | 1.00 | 148.42 |
| 1707 | C | ASP | B | 347 | 68.509 | 119.576 | 158.466 | 1.00 | 78.62 |
| 1708 | O | ASP | B | 347 | 68.339 | 120.174 | 157.401 | 1.00 | 40.12 |
| 1709 | N | LEU | B | 348 | 68.969 | 120.178 | 159.550 | 1.00 | 43.72 |
| 1710 | CA | LEU | B | 348 | 69.184 | 121.613 | 159.553 | 1.00 | 79.44 |
| 1711 | CB | LEU | B | 348 | 68.570 | 122.183 | 160.837 | 1.00 | 64.43 |
| 1712 | CG | LEU | B | 348 | 68.601 | 123.691 | 161.101 | 1.00 | 74.91 |
| 1713 | CD1 | LEU | B | 348 | 68.208 | 124.482 | 159.860 | 1.00 | 80.89 |
| 1714 | CD2 | LEU | B | 348 | 67.656 | 123.985 | 162.246 | 1.00 | 82.45 |
| 1715 | C | LEU | B | 348 | 70.617 | 122.094 | 159.396 | 1.00 | 77.53 |
| 1716 | O | LEU | B | 348 | 70.863 | 123.165 | 158.841 | 1.00 | 66.37 |
| 1717 | N | PHE | B | 349 | 71.560 | 121.300 | 159.877 | 1.00 | 81.75 |
| 1718 | CA | PHE | B | 349 | 72.964 | 121.666 | 159.811 | 1.00 | 90.94 |
| 1719 | CB | PHE | B | 349 | 73.515 | 121.657 | 161.221 | 1.00 | 89.74 |
| 1720 | CG | PHE | B | 349 | 72.864 | 122.665 | 162.096 | 1.00 | 88.86 |
| 1721 | CD1 | PHE | B | 349 | 72.745 | 122.458 | 163.464 | 1.00 | 102.92 |
| 1722 | CD2 | PHE | B | 349 | 72.363 | 123.837 | 161.542 | 1.00 | 48.94 |
| 1723 | CE1 | PHE | B | 349 | 72.133 | 123.406 | 164.274 | 1.00 | 64.93 |
| 1724 | CE2 | PHE | B | 349 | 71.754 | 124.786 | 162.335 | 1.00 | 94.98 |
| 1725 | CZ | PHE | B | 349 | 71.638 | 124.572 | 163.708 | 1.00 | 102.89 |
| 1726 | C | PHE | B | 349 | 73.806 | 120.800 | 158.889 | 1.00 | 102.94 |
| 1727 | O | PHE | B | 349 | 74.633 | 121.304 | 158.137 | 1.00 | 120.26 |
| 1728 | N | ILE | B | 350 | 73.609 | 119.494 | 158.963 | 1.00 | 100.45 |
| 1729 | CA | ILE | B | 350 | 74.328 | 118.586 | 158.098 | 1.00 | 57.80 |
| 1730 | CB | ILE | B | 350 | 74.145 | 117.136 | 158.545 | 1.00 | 71.44 |
| 1731 | CG2 | ILE | B | 350 | 74.830 | 116.213 | 157.588 | 1.00 | 91.54 |
| 1732 | CG1 | ILE | B | 350 | 74.662 | 116.959 | 159.972 | 1.00 | 71.63 |
| 1733 | CD1 | ILE | B | 350 | 76.040 | 117.471 | 160.193 | 1.00 | 65.43 |
| 1734 | C | ILE | B | 350 | 73.672 | 118.752 | 156.738 | 1.00 | 76.53 |
| 1735 | O | ILE | B | 350 | 74.101 | 119.559 | 155.928 | 1.00 | 93.37 |
| 1736 | N | ARG | B | 351 | 72.601 | 117.998 | 156.520 | 1.00 | 76.58 |
| 1737 | CA | ARG | B | 351 | 71.852 | 118.003 | 155.261 | 1.00 | 85.35 |
| 1738 | CB | ARG | B | 351 | 70.544 | 117.223 | 155.464 | 1.00 | 94.17 |
| 1739 | CG | ARG | B | 351 | 69.978 | 116.539 | 154.229 | 1.00 | 114.26 |
| 1740 | CD | ARG | B | 351 | 69.081 | 115.375 | 154.644 | 1.00 | 134.12 |
| 1741 | NE | ARG | B | 351 | 68.530 | 114.661 | 153.497 | 1.00 | 180.07 |
| 1742 | CZ | ARG | B | 351 | 67.646 | 115.179 | 152.650 | 1.00 | 196.67 |
| 1743 | NH1 | ARG | B | 351 | 67.210 | 116.420 | 152.821 | 1.00 | 201.25 |
| 1744 | NH2 | ARG | B | 351 | 67.198 | 114.456 | 151.632 | 1.00 | 193.34 |
| 1745 | C | ARG | B | 351 | 71.563 | 119.406 | 154.722 | 1.00 | 85.97 |
| 1746 | O | ARG | B | 351 | 71.257 | 119.576 | 153.546 | 1.00 | 79.18 |
| 1747 | N | LYS | B | 352 | 71.672 | 120.401 | 155.594 | 1.00 | 70.30 |
| 1748 | CA | LYS | B | 352 | 71.417 | 121.800 | 155.249 | 1.00 | 116.58 |
| 1749 | CB | LYS | B | 352 | 72.641 | 122.404 | 154.559 | 1.00 | 144.96 |
| 1750 | CG | LYS | B | 352 | 73.881 | 122.501 | 155.448 | 1.00 | 168.84 |
| 1751 | CD | LYS | B | 352 | 74.894 | 123.491 | 154.874 | 1.00 | 186.41 |
| 1752 | CE | LYS | B | 352 | 76.217 | 123.484 | 155.633 | 1.00 | 172.60 |
| 1753 | NZ | LYS | B | 352 | 77.028 | 122.264 | 155.354 | 1.00 | 186.13 |
| 1754 | C | LYS | B | 352 | 70.162 | 122.102 | 154.416 | 1.00 | 119.94 |
| 1755 | O | LYS | B | 352 | 70.110 | 123.108 | 153.709 | 1.00 | 105.59 |
| 1756 | N | SER | B | 353 | 69.160 | 121.231 | 154.504 | 1.00 | 128.80 |
| 1757 | CA | SER | B | 353 | 67.884 | 121.411 | 153.802 | 1.00 | 102.46 |
| 1758 | CB | SER | B | 353 | 67.788 | 120.501 | 152.574 | 1.00 | 122.75 |
| 1759 | OG | SER | B | 353 | 67.784 | 119.131 | 152.936 | 1.00 | 168.70 |
| 1760 | C | SER | B | 353 | 66.811 | 121.034 | 154.818 | 1.00 | 99.64 |
| 1761 | O | SER | B | 353 | 66.337 | 119.897 | 154.860 | 1.00 | 93.09 |
| 1762 | N | PRO | B | 354 | 66.421 | 121.994 | 155.661 | 1.00 | 84.24 |
| 1763 | CD | PRO | B | 354 | 66.948 | 123.357 | 155.738 | 1.00 | 97.02 |
| 1764 | CA | PRO | B | 354 | 65.415 | 121.793 | 156.698 | 1.00 | 77.36 |
| 1765 | CB | PRO | B | 354 | 65.720 | 122.910 | 157.702 | 1.00 | 87.64 |
| 1766 | CG | PRO | B | 354 | 67.006 | 123.540 | 157.208 | 1.00 | 75.30 |
| 1767 | C | PRO | B | 354 | 63.998 | 121.894 | 156.181 | 1.00 | 95.66 |
| 1768 | O | PRO | B | 354 | 63.722 | 122.632 | 155.225 | 1.00 | 61.77 |
| 1769 | N | THR | B | 355 | 63.108 | 121.145 | 156.826 | 1.00 | 83.51 |
| 1770 | CA | THR | B | 355 | 61.696 | 121.153 | 156.478 | 1.00 | 60.49 |
| 1771 | CB | THR | B | 355 | 61.340 | 120.121 | 155.405 | 1.00 | 59.24 |
| 1772 | OG1 | THR | B | 355 | 61.685 | 118.810 | 155.876 | 1.00 | 77.07 |
| 1773 | CG2 | THR | B | 355 | 62.053 | 120.440 | 154.098 | 1.00 | 114.79 |
| 1774 | C | THR | B | 355 | 60.837 | 120.838 | 157.683 | 1.00 | 79.73 |
| 1775 | O | THR | B | 355 | 61.132 | 119.924 | 158.464 | 1.00 | 73.10 |
| 1776 | N | ILE | B | 356 | 59.765 | 121.613 | 157.815 | 1.00 | 73.22 |
| 1777 | CA | ILE | B | 356 | 58.801 | 121.449 | 158.891 | 1.00 | 76.70 |
| 1778 | CB | ILE | B | 356 | 58.351 | 122.805 | 159.441 | 1.00 | 47.99 |
| 1779 | CG2 | ILE | B | 356 | 59.496 | 123.453 | 160.194 | 1.00 | 83.94 |
| 1780 | CG1 | ILE | B | 356 | 57.861 | 123.691 | 158.294 | 1.00 | 66.86 |
| 1781 | CD1 | ILE | B | 356 | 57.478 | 125.075 | 158.729 | 1.00 | 80.08 |
| 1782 | C | ILE | B | 356 | 57.595 | 120.723 | 158.311 | 1.00 | 74.21 |
| 1783 | O | ILE | B | 356 | 57.290 | 120.849 | 157.110 | 1.00 | 45.14 |
| 1784 | N | THR | B | 357 | 56.898 | 119.978 | 159.164 | 1.00 | 59.52 |
| 1785 | CA | THR | B | 357 | 55.752 | 119.215 | 158.706 | 1.00 | 66.63 |
| 1786 | CB | THR | B | 357 | 56.095 | 117.748 | 158.697 | 1.00 | 60.44 |
| 1787 | OG1 | THR | B | 357 | 57.388 | 117.574 | 158.106 | 1.00 | 96.24 |
| 1788 | CG2 | THR | B | 357 | 55.066 | 116.981 | 157.904 | 1.00 | 78.11 |
| 1789 | C | THR | B | 357 | 54.494 | 119.395 | 159.534 | 1.00 | 65.25 |
| 1790 | O | THR | B | 357 | 54.525 | 119.290 | 160.762 | 1.00 | 62.91 |
| 1791 | N | CYS | B | 358 | 53.387 | 119.639 | 158.836 | 1.00 | 60.07 |
| 1792 | CA | CYS | B | 358 | 52.076 | 119.835 | 159.453 | 1.00 | 60.41 |
| 1793 | C | CYS | B | 358 | 51.260 | 118.568 | 159.245 | 1.00 | 47.28 |
| 1794 | O | CYS | B | 358 | 50.999 | 118.164 | 158.117 | 1.00 | 61.76 |
| 1795 | CB | CYS | B | 358 | 51.372 | 121.006 | 158.789 | 1.00 | 66.79 |
| 1796 | SG | CYS | B | 358 | 49.884 | 121.601 | 159.632 | 1.00 | 87.73 |
| 1797 | N | LEU | B | 359 | 50.862 | 117.934 | 160.334 | 1.00 | 55.07 |
| 1798 | CA | LEU | B | 359 | 50.102 | 116.700 | 160.248 | 1.00 | 25.58 |
| 1799 | CB | LEU | B | 359 | 50.884 | 115.602 | 160.956 | 1.00 | 60.22 |
| 1800 | CG | LEU | B | 359 | 50.116 | 114.353 | 161.371 | 1.00 | 17.72 |
| 1801 | CD1 | LEU | B | 359 | 49.435 | 113.786 | 160.175 | 1.00 | 49.40 |
| 1802 | CD2 | LEU | B | 359 | 51.037 | 113.345 | 162.002 | 1.00 | 86.18 |
| 1803 | C | LEU | B | 359 | 48.703 | 116.836 | 160.859 | 1.00 | 39.46 |
| 1804 | O | LEU | B | 359 | 48.538 | 117.114 | 162.044 | 1.00 | 57.72 |
| 1805 | N | VAL | B | 360 | 47.692 | 116.631 | 160.034 | 1.00 | 50.02 |
| 1806 | CA | VAL | B | 360 | 46.316 | 116.727 | 160.476 | 1.00 | 35.99 |
| 1807 | CB | VAL | B | 360 | 45.467 | 117.468 | 159.452 | 1.00 | 46.54 |
| 1808 | CG1 | VAL | B | 360 | 44.028 | 117.436 | 159.862 | 1.00 | 47.57 |
| 1809 | CG2 | VAL | B | 360 | 45.948 | 118.886 | 159.328 | 1.00 | 29.82 |
| 1810 | C | VAL | B | 360 | 45.751 | 115.334 | 160.628 | 1.00 | 34.08 |
| 1811 | O | VAL | B | 360 | 45.885 | 114.501 | 159.733 | 1.00 | 60.52 |
| 1812 | N | VAL | B | 361 | 45.102 | 115.091 | 161.759 | 1.00 | 22.24 |
| 1813 | CA | VAL | B | 361 | 44.520 | 113.790 | 162.040 | 1.00 | 37.22 |
| 1814 | CB | VAL | B | 361 | 45.305 | 113.117 | 163.163 | 1.00 | 11.28 |
| 1815 | CG1 | VAL | B | 361 | 46.626 | 113.793 | 163.312 | 1.00 | 37.36 |
| 1816 | CG2 | VAL | B | 361 | 44.563 | 113.194 | 164.430 | 1.00 | 36.29 |
| 1817 | C | VAL | B | 361 | 43.032 | 113.828 | 162.424 | 1.00 | 32.87 |
| 1818 | O | VAL | B | 361 | 42.504 | 114.859 | 162.814 | 1.00 | 47.67 |
| 1819 | N | ASP | B | 362 | 42.374 | 112.680 | 162.324 | 1.00 | 50.85 |
| 1820 | CA | ASP | B | 362 | 40.963 | 112.540 | 162.657 | 1.00 | 28.87 |
| 1821 | CB | ASP | B | 362 | 40.697 | 112.967 | 164.092 | 1.00 | 43.13 |
| 1822 | CG | ASP | B | 362 | 41.283 | 111.999 | 165.107 | 1.00 | 96.71 |
| 1823 | OD1 | ASP | B | 362 | 41.186 | 110.772 | 164.910 | 1.00 | 61.11 |
| 1824 | OD2 | ASP | B | 362 | 41.832 | 112.464 | 166.122 | 1.00 | 95.29 |
| 1825 | C | ASP | B | 362 | 39.978 | 113.227 | 161.726 | 1.00 | 39.54 |
| 1826 | O | ASP | B | 362 | 38.838 | 113.482 | 162.112 | 1.00 | 44.37 |
| 1827 | N | LEU | B | 363 | 40.410 | 113.513 | 160.500 | 1.00 | 30.82 |
| 1828 | CA | LEU | B | 363 | 39.536 | 114.118 | 159.506 | 1.00 | 21.83 |
| 1829 | CB | LEU | B | 363 | 40.328 | 114.589 | 158.298 | 1.00 | 31.47 |
| 1830 | CG | LEU | B | 363 | 41.130 | 115.877 | 158.418 | 1.00 | 45.22 |
| 1831 | CD1 | LEU | B | 363 | 42.030 | 116.037 | 157.201 | 1.00 | 61.55 |
| 1832 | CD2 | LEU | B | 363 | 40.174 | 117.029 | 158.514 | 1.00 | 33.26 |
| 1833 | C | LEU | B | 363 | 38.608 | 113.026 | 159.049 | 1.00 | 44.60 |
| 1834 | O | LEU | B | 363 | 38.922 | 111.859 | 159.163 | 1.00 | 46.41 |
| 1835 | N | ALA | B | 364 | 37.455 | 113.392 | 158.533 | 1.00 | 51.90 |
| 1836 | CA | ALA | B | 364 | 36.541 | 112.383 | 158.045 | 1.00 | 22.74 |
| 1837 | CB | ALA | B | 364 | 35.186 | 112.587 | 158.647 | 1.00 | 51.33 |
| 1838 | C | ALA | B | 364 | 36.464 | 112.500 | 156.530 | 1.00 | 60.62 |
| 1839 | O | ALA | B | 364 | 36.529 | 113.603 | 155.972 | 1.00 | 44.38 |
| 1840 | N | PRO | B | 365 | 36.339 | 111.365 | 155.837 | 1.00 | 18.39 |
| 1841 | CD | PRO | B | 365 | 35.980 | 110.033 | 156.333 | 1.00 | 51.04 |
| 1842 | CA | PRO | B | 365 | 36.255 | 111.404 | 154.379 | 1.00 | 40.07 |
| 1843 | CB | PRO | B | 365 | 35.930 | 109.963 | 154.027 | 1.00 | 109.08 |
| 1844 | CG | PRO | B | 365 | 35.117 | 109.531 | 155.214 | 1.00 | 40.87 |
| 1845 | C | PRO | B | 365 | 35.132 | 112.340 | 153.965 | 1.00 | 58.17 |
| 1846 | O | PRO | B | 365 | 34.127 | 112.428 | 154.672 | 1.00 | 50.80 |
| 1847 | N | SER | B | 366 | 35.303 | 113.028 | 152.833 | 1.00 | 35.27 |
| 1848 | CA | SER | B | 366 | 34.292 | 113.944 | 152.315 | 1.00 | 93.15 |
| 1849 | CB | SER | B | 366 | 34.314 | 115.271 | 153.076 | 1.00 | 108.06 |
| 1850 | OG | SER | B | 366 | 35.515 | 115.977 | 152.855 | 1.00 | 71.65 |
| 1851 | C | SER | B | 366 | 34.515 | 114.210 | 150.839 | 1.00 | 44.93 |
| 1852 | O | SER | B | 366 | 35.556 | 113.866 | 150.290 | 1.00 | 106.10 |
| 1853 | N | LYS | B | 367 | 33.529 | 114.827 | 150.198 | 1.00 | 130.17 |
| 1854 | CA | LYS | B | 367 | 33.600 | 115.155 | 148.779 | 1.00 | 79.40 |
| 1855 | CB | LYS | B | 367 | 32.319 | 115.878 | 148.331 | 1.00 | 125.03 |
| 1856 | CG | LYS | B | 367 | 31.050 | 115.020 | 148.296 | 1.00 | 164.89 |
| 1857 | CD | LYS | B | 367 | 29.862 | 115.815 | 147.732 | 1.00 | 160.83 |
| 1858 | CE | LYS | B | 367 | 28.612 | 114.952 | 147.548 | 1.00 | 137.20 |
| 1859 | NZ | LYS | B | 367 | 27.489 | 115.720 | 146.924 | 1.00 | 126.69 |
| 1860 | C | LYS | B | 367 | 34.806 | 116.040 | 148.477 | 1.00 | 52.10 |
| 1861 | O | LYS | B | 367 | 35.562 | 115.774 | 147.544 | 1.00 | 108.75 |
| 1862 | N | GLY | B | 368 | 34.986 | 117.089 | 149.272 | 1.00 | 66.48 |
| 1863 | CA | GLY | B | 368 | 36.093 | 117.999 | 149.043 | 1.00 | 81.03 |
| 1864 | C | GLY | B | 368 | 37.267 | 117.768 | 149.965 | 1.00 | 56.47 |
| 1865 | O | GLY | B | 368 | 37.106 | 117.247 | 151.062 | 1.00 | 92.39 |
| 1866 | N | THR | B | 369 | 38.455 | 118.154 | 149.516 | 1.00 | 59.57 |
| 1867 | CA | THR | B | 369 | 39.659 | 117.986 | 150.313 | 1.00 | 61.18 |
| 1868 | CB | THR | B | 369 | 40.891 | 117.867 | 149.416 | 1.00 | 71.21 |
| 1869 | OG1 | THR | B | 369 | 41.072 | 119.088 | 148.693 | 1.00 | 96.74 |
| 1870 | CG2 | THR | B | 369 | 40.710 | 116.731 | 148.420 | 1.00 | 106.84 |
| 1871 | C | THR | B | 369 | 39.802 | 119.215 | 151.181 | 1.00 | 47.36 |
| 1872 | O | THR | B | 369 | 39.091 | 120.193 | 150.985 | 1.00 | 60.95 |
| 1873 | N | VAL | B | 370 | 40.712 | 119.183 | 152.142 | 1.00 | 34.09 |
| 1874 | CA | VAL | B | 370 | 40.888 | 120.349 | 152.999 | 1.00 | 48.60 |
| 1875 | CB | VAL | B | 370 | 41.744 | 120.043 | 154.219 | 1.00 | 24.99 |
| 1876 | CG1 | VAL | B | 370 | 41.249 | 118.771 | 154.883 | 1.00 | 36.87 |
| 1877 | CG2 | VAL | B | 370 | 43.201 | 119.920 | 153.814 | 1.00 | 68.00 |
| 1878 | C | VAL | B | 370 | 41.552 | 121.468 | 152.225 | 1.00 | 25.41 |
| 1879 | O | VAL | B | 370 | 41.788 | 121.345 | 151.027 | 1.00 | 88.63 |
| 1880 | N | ASN | B | 371 | 41.866 | 122.554 | 152.915 | 1.00 | 53.72 |
| 1881 | CA | ASN | B | 371 | 42.477 | 123.715 | 152.290 | 1.00 | 52.65 |
| 1882 | CB | ASN | B | 371 | 41.401 | 124.794 | 152.091 | 1.00 | 74.57 |
| 1883 | CG | ASN | B | 371 | 41.906 | 126.001 | 151.353 | 1.00 | 115.17 |
| 1884 | OD1 | ASN | B | 371 | 43.097 | 126.127 | 151.082 | 1.00 | 164.46 |
| 1885 | ND2 | ASN | B | 371 | 40.996 | 126.917 | 151.034 | 1.00 | 174.36 |
| 1886 | C | ASN | B | 371 | 43.581 | 124.172 | 153.235 | 1.00 | 58.62 |
| 1887 | O | ASN | B | 371 | 43.409 | 125.079 | 154.042 | 1.00 | 79.32 |
| 1888 | N | LEU | B | 372 | 44.713 | 123.489 | 153.130 | 1.00 | 85.89 |
| 1889 | CA | LEU | B | 372 | 45.903 | 123.722 | 153.936 | 1.00 | 45.57 |
| 1890 | CB | LEU | B | 372 | 46.816 | 122.511 | 153.816 | 1.00 | 37.74 |
| 1891 | CG | LEU | B | 372 | 47.914 | 122.228 | 154.841 | 1.00 | 36.14 |
| 1892 | CD1 | LEU | B | 372 | 48.335 | 123.493 | 155.586 | 1.00 | 42.25 |
| 1893 | CD2 | LEU | B | 372 | 47.373 | 121.176 | 155.809 | 1.00 | 26.23 |
| 1894 | C | LEU | B | 372 | 46.605 | 124.937 | 153.369 | 1.00 | 69.02 |
| 1895 | O | LEU | B | 372 | 46.702 | 125.071 | 152.155 | 1.00 | 96.74 |
| 1896 | N | THR | B | 373 | 47.102 | 125.818 | 154.228 | 1.00 | 57.25 |
| 1897 | CA | THR | B | 373 | 47.760 | 127.025 | 153.748 | 1.00 | 31.14 |
| 1898 | CB | THR | B | 373 | 46.741 | 128.177 | 153.665 | 1.00 | 67.35 |
| 1899 | OG1 | THR | B | 373 | 45.708 | 127.832 | 152.735 | 1.00 | 72.32 |
| 1900 | CG2 | THR | B | 373 | 47.397 | 129.452 | 153.211 | 1.00 | 83.78 |
| 1901 | C | THR | B | 373 | 48.927 | 127.440 | 154.633 | 1.00 | 76.56 |
| 1902 | O | THR | B | 373 | 48.768 | 127.635 | 155.846 | 1.00 | 63.76 |
| 1903 | N | TRP | B | 374 | 50.099 | 127.578 | 154.010 | 1.00 | 81.63 |
| 1904 | CA | TRP | B | 374 | 51.325 | 127.962 | 154.715 | 1.00 | 63.39 |
| 1905 | CB | TRP | B | 374 | 52.533 | 127.246 | 154.128 | 1.00 | 60.06 |
| 1906 | CG | TRP | B | 374 | 52.577 | 125.801 | 154.393 | 1.00 | 38.27 |
| 1907 | CD2 | TRP | B | 374 | 53.047 | 125.173 | 155.583 | 1.00 | 17.81 |
| 1908 | CE2 | TRP | B | 374 | 52.925 | 123.781 | 155.398 | 1.00 | 26.57 |
| 1909 | CE3 | TRP | B | 374 | 53.563 | 125.650 | 156.784 | 1.00 | 45.86 |
| 1910 | CD1 | TRP | B | 374 | 52.195 | 124.796 | 153.549 | 1.00 | 58.25 |
| 1911 | NE1 | TRP | B | 374 | 52.403 | 123.575 | 154.149 | 1.00 | 41.78 |
| 1912 | CZ2 | TRP | B | 374 | 53.300 | 122.866 | 156.373 | 1.00 | 46.60 |
| 1913 | CZ3 | TRP | B | 374 | 53.940 | 124.734 | 157.759 | 1.00 | 44.09 |
| 1914 | CH2 | TRP | B | 374 | 53.804 | 123.364 | 157.548 | 1.00 | 18.32 |
| 1915 | C | TRP | B | 374 | 51.591 | 129.457 | 154.649 | 1.00 | 94.67 |
| 1916 | O | TRP | B | 374 | 51.341 | 130.096 | 153.622 | 1.00 | 70.49 |
| 1917 | N | SER | B | 375 | 52.133 | 130.003 | 155.735 | 1.00 | 79.04 |
| 1918 | CA | SER | B | 375 | 52.421 | 131.428 | 155.798 | 1.00 | 76.64 |
| 1919 | CB | SER | B | 375 | 51.136 | 132.190 | 156.128 | 1.00 | 121.29 |
| 1920 | OG | SER | B | 375 | 50.563 | 131.725 | 157.343 | 1.00 | 115.00 |
| 1921 | C | SER | B | 375 | 53.500 | 131.793 | 156.818 | 1.00 | 100.83 |
| 1922 | O | SER | B | 375 | 53.681 | 131.114 | 157.844 | 1.00 | 45.77 |
| 1923 | N | ARG | B | 376 | 54.215 | 132.876 | 156.526 | 1.00 | 84.64 |
| 1924 | CA | ARG | B | 376 | 55.263 | 133.366 | 157.418 | 1.00 | 89.30 |
| 1925 | CB | ARG | B | 376 | 56.525 | 133.715 | 156.631 | 1.00 | 108.51 |
| 1926 | CG | ARG | B | 376 | 57.394 | 132.526 | 156.294 | 1.00 | 125.68 |
| 1927 | CD | ARG | B | 376 | 58.852 | 132.940 | 156.133 | 1.00 | 127.76 |
| 1928 | NE | ARG | B | 376 | 59.165 | 133.476 | 154.812 | 1.00 | 88.17 |
| 1929 | CZ | ARG | B | 376 | 60.372 | 133.898 | 154.461 | 1.00 | 124.32 |
| 1930 | NH1 | ARG | B | 376 | 61.368 | 133.849 | 155.337 | 1.00 | 105.92 |
| 1931 | NH2 | ARG | B | 376 | 60.588 | 134.349 | 153.234 | 1.00 | 164.32 |
| 1932 | C | ARG | B | 376 | 54.795 | 134.607 | 158.167 | 1.00 | 120.27 |
| 1933 | O | ARG | B | 376 | 53.953 | 135.367 | 157.674 | 1.00 | 114.07 |
| 1934 | N | ALA | B | 377 | 55.344 | 134.817 | 159.357 | 1.00 | 101.03 |
| 1935 | CA | ALA | B | 377 | 54.973 | 135.981 | 160.151 | 1.00 | 108.48 |
| 1936 | CB | ALA | B | 377 | 55.394 | 135.784 | 161.583 | 1.00 | 88.56 |
| 1937 | C | ALA | B | 377 | 55.607 | 137.252 | 159.593 | 1.00 | 112.40 |
| 1938 | O | ALA | B | 377 | 55.071 | 138.345 | 159.751 | 1.00 | 124.37 |
| 1939 | N | SER | B | 378 | 56.753 | 137.105 | 158.943 | 1.00 | 101.18 |
| 1940 | CA | SER | B | 378 | 57.445 | 138.242 | 158.362 | 1.00 | 69.51 |
| 1941 | CB | SER | B | 378 | 58.845 | 137.831 | 157.902 | 1.00 | 111.91 |
| 1942 | OG | SER | B | 378 | 58.778 | 137.003 | 156.747 | 1.00 | 103.91 |
| 1943 | C | SER | B | 378 | 56.665 | 138.786 | 157.166 | 1.00 | 87.46 |
| 1944 | O | SER | B | 378 | 56.842 | 139.936 | 156.769 | 1.00 | 118.01 |
| 1945 | N | GLY | B | 379 | 55.807 | 137.957 | 156.588 | 1.00 | 90.70 |
| 1946 | CA | GLY | B | 379 | 55.031 | 138.392 | 155.441 | 1.00 | 104.68 |
| 1947 | C | GLY | B | 379 | 55.679 | 137.954 | 154.143 | 1.00 | 122.51 |
| 1948 | O | GLY | B | 379 | 55.045 | 137.946 | 153.084 | 1.00 | 110.08 |
| 1949 | N | LYS | B | 380 | 56.950 | 137.579 | 154.229 | 1.00 | 122.20 |
| 1950 | CA | LYS | B | 380 | 57.699 | 137.136 | 153.061 | 1.00 | 147.83 |
| 1951 | CB | LYS | B | 380 | 59.174 | 136.966 | 153.428 | 1.00 | 162.29 |
| 1952 | CG | LYS | B | 380 | 59.830 | 138.223 | 153.989 | 1.00 | 173.82 |
| 1953 | CD | LYS | B | 380 | 61.286 | 137.969 | 154.360 | 1.00 | 180.37 |
| 1954 | CE | LYS | B | 380 | 61.949 | 139.218 | 154.922 | 1.00 | 172.23 |
| 1955 | NZ | LYS | B | 380 | 63.378 | 138.976 | 155.269 | 1.00 | 155.65 |
| 1956 | C | LYS | B | 380 | 57.145 | 135.820 | 152.513 | 1.00 | 146.60 |
| 1957 | O | LYS | B | 380 | 56.856 | 134.897 | 153.270 | 1.00 | 157.16 |
| 1958 | N | PRO | B | 381 | 56.992 | 135.724 | 151.182 | 1.00 | 140.30 |
| 1959 | CD | PRO | B | 381 | 57.285 | 136.796 | 150.216 | 1.00 | 154.76 |
| 1960 | CA | PRO | B | 381 | 56.475 | 134.537 | 150.493 | 1.00 | 131.70 |
| 1961 | CB | PRO | B | 381 | 56.787 | 134.838 | 149.034 | 1.00 | 142.42 |
| 1962 | CG | PRO | B | 381 | 56.572 | 136.313 | 148.971 | 1.00 | 139.64 |
| 1963 | C | PRO | B | 381 | 57.085 | 133.216 | 150.966 | 1.00 | 119.70 |
| 1964 | O | PRO | B | 381 | 58.115 | 133.207 | 151.647 | 1.00 | 96.93 |
| 1965 | N | VAL | B | 382 | 56.443 | 132.109 | 150.589 | 1.00 | 110.70 |
| 1966 | CA | VAL | B | 382 | 56.885 | 130.765 | 150.975 | 1.00 | 88.04 |
| 1967 | CB | VAL | B | 382 | 55.908 | 130.140 | 151.964 | 1.00 | 67.75 |
| 1968 | CG1 | VAL | B | 382 | 55.938 | 130.895 | 153.273 | 1.00 | 128.08 |
| 1969 | CG2 | VAL | B | 382 | 54.511 | 130.160 | 151.360 | 1.00 | 82.52 |
| 1970 | C | VAL | B | 382 | 57.020 | 129.784 | 149.808 | 1.00 | 109.58 |
| 1971 | O | VAL | B | 382 | 56.279 | 129.858 | 148.817 | 1.00 | 109.44 |
| 1972 | N | ASN | B | 383 | 57.958 | 128.849 | 149.947 | 1.00 | 109.75 |
| 1973 | CA | ASN | B | 383 | 58.197 | 127.845 | 148.918 | 1.00 | 128.69 |
| 1974 | CB | ASN | B | 383 | 59.393 | 126.966 | 149.309 | 1.00 | 135.40 |
| 1975 | CG | ASN | B | 383 | 60.723 | 127.696 | 149.189 | 1.00 | 146.65 |
| 1976 | OD1 | ASN | B | 383 | 61.759 | 127.196 | 149.629 | 1.00 | 133.04 |
| 1977 | ND2 | ASN | B | 383 | 60.702 | 128.877 | 148.582 | 1.00 | 137.22 |
| 1978 | C | ASN | B | 383 | 56.955 | 126.981 | 148.730 | 1.00 | 91.22 |
| 1979 | O | ASN | B | 383 | 56.024 | 127.038 | 149.524 | 1.00 | 98.35 |
| 1980 | N | HIS | B | 384 | 56.937 | 126.189 | 147.668 | 1.00 | 93.62 |
| 1981 | CA | HIS | B | 384 | 55.806 | 125.312 | 147.411 | 1.00 | 67.76 |
| 1982 | CB | HIS | B | 384 | 55.861 | 124.821 | 145.971 | 1.00 | 86.21 |
| 1983 | CG | HIS | B | 384 | 55.759 | 125.923 | 144.968 | 1.00 | 106.64 |
| 1984 | CD2 | HIS | B | 384 | 56.710 | 126.691 | 144.388 | 1.00 | 118.02 |
| 1985 | ND1 | HIS | B | 384 | 54.551 | 126.390 | 144.500 | 1.00 | 70.58 |
| 1986 | CE1 | HIS | B | 384 | 54.760 | 127.398 | 143.676 | 1.00 | 107.89 |
| 1987 | NE2 | HIS | B | 384 | 56.063 | 127.602 | 143.590 | 1.00 | 141.17 |
| 1988 | C | HIS | B | 384 | 55.859 | 124.145 | 148.392 | 1.00 | 78.76 |
| 1989 | O | HIS | B | 384 | 56.936 | 123.688 | 148.786 | 1.00 | 62.48 |
| 1990 | N | SER | B | 385 | 54.694 | 123.665 | 148.801 | 1.00 | 90.92 |
| 1991 | CA | SER | B | 385 | 54.650 | 122.571 | 149.760 | 1.00 | 50.91 |
| 1992 | CB | SER | B | 385 | 53.664 | 122.908 | 150.880 | 1.00 | 71.25 |
| 1993 | OG | SER | B | 385 | 52.375 | 123.192 | 150.354 | 1.00 | 104.23 |
| 1994 | C | SER | B | 385 | 54.271 | 121.242 | 149.136 | 1.00 | 72.39 |
| 1995 | O | SER | B | 385 | 53.913 | 121.162 | 147.955 | 1.00 | 65.67 |
| 1996 | N | THR | B | 386 | 54.359 | 120.210 | 149.970 | 1.00 | 62.20 |
| 1997 | CA | THR | B | 386 | 54.036 | 118.826 | 149.632 | 1.00 | 53.65 |
| 1998 | CB | THR | B | 386 | 55.117 | 117.911 | 150.210 | 1.00 | 90.34 |
| 1999 | OG1 | THR | B | 386 | 56.193 | 117.804 | 149.269 | 1.00 | 80.69 |
| 2000 | CG2 | THR | B | 386 | 54.552 | 116.533 | 150.571 | 1.00 | 100.14 |
| 2001 | C | THR | B | 386 | 52.684 | 118.468 | 150.266 | 1.00 | 68.47 |
| 2002 | O | THR | B | 386 | 52.200 | 119.188 | 151.139 | 1.00 | 105.23 |
| 2003 | N | ARG | B | 387 | 52.063 | 117.377 | 149.837 | 1.00 | 56.81 |
| 2004 | CA | ARG | B | 387 | 50.795 | 117.008 | 150.437 | 1.00 | 54.80 |
| 2005 | CB | ARG | B | 387 | 49.720 | 118.013 | 150.006 | 1.00 | 49.15 |
| 2006 | CG | ARG | B | 387 | 48.321 | 117.671 | 150.466 | 1.00 | 66.87 |
| 2007 | CD | ARG | B | 387 | 47.403 | 118.853 | 150.339 | 1.00 | 67.37 |
| 2008 | NE | ARG | B | 387 | 46.030 | 118.490 | 150.645 | 1.00 | 77.86 |
| 2009 | CZ | ARG | B | 387 | 45.055 | 119.376 | 150.780 | 1.00 | 111.21 |
| 2010 | NH1 | ARG | B | 387 | 45.318 | 120.669 | 150.633 | 1.00 | 100.94 |
| 2011 | NH2 | ARG | B | 387 | 43.825 | 118.971 | 151.064 | 1.00 | 96.41 |
| 2012 | C | ARG | B | 387 | 50.347 | 115.582 | 150.128 | 1.00 | 56.65 |
| 2013 | O | ARG | B | 387 | 50.294 | 115.184 | 148.963 | 1.00 | 61.16 |
| 2014 | N | LYS | B | 388 | 50.035 | 114.816 | 151.177 | 1.00 | 36.92 |
| 2015 | CA | LYS | B | 388 | 49.555 | 113.441 | 151.016 | 1.00 | 59.67 |
| 2016 | CB | LYS | B | 388 | 50.560 | 112.426 | 151.556 | 1.00 | 30.37 |
| 2017 | CG | LYS | B | 388 | 52.019 | 112.796 | 151.452 | 1.00 | 148.33 |
| 2018 | CD | LYS | B | 388 | 52.864 | 111.704 | 152.106 | 1.00 | 145.55 |
| 2019 | CE | LYS | B | 388 | 54.353 | 112.005 | 152.019 | 1.00 | 171.95 |
| 2020 | NZ | LYS | B | 388 | 55.174 | 110.852 | 152.491 | 1.00 | 153.54 |
| 2021 | C | LYS | B | 388 | 48.290 | 113.253 | 151.835 | 1.00 | 56.48 |
| 2022 | O | LYS | B | 388 | 48.189 | 113.793 | 152.938 | 1.00 | 73.87 |
| 2023 | N | GLU | B | 389 | 47.339 | 112.480 | 151.324 | 1.00 | 33.78 |
| 2024 | CA | GLU | B | 389 | 46.120 | 112.216 | 152.086 | 1.00 | 52.94 |
| 2025 | CB | GLU | B | 389 | 44.889 | 112.865 | 151.423 | 1.00 | 32.75 |
| 2026 | CG | GLU | B | 389 | 44.856 | 114.388 | 151.542 | 1.00 | 138.12 |
| 2027 | CD | GLU | B | 389 | 43.709 | 115.017 | 150.776 | 1.00 | 167.38 |
| 2028 | OE1 | GLU | B | 389 | 42.548 | 114.596 | 150.983 | 1.00 | 151.81 |
| 2029 | OE2 | GLU | B | 389 | 43.977 | 115.938 | 149.971 | 1.00 | 152.33 |
| 2030 | C | GLU | B | 389 | 45.927 | 110.715 | 152.197 | 1.00 | 54.81 |
| 2031 | O | GLU | B | 389 | 45.401 | 110.088 | 151.280 | 1.00 | 91.54 |
| 2032 | N | GLU | B | 390 | 46.345 | 110.143 | 153.321 | 1.00 | 39.47 |
| 2033 | CA | GLU | B | 390 | 46.227 | 108.705 | 153.522 | 1.00 | 78.09 |
| 2034 | CB | GLU | B | 390 | 47.466 | 108.168 | 154.252 | 1.00 | 97.18 |
| 2035 | CG | GLU | B | 390 | 48.812 | 108.585 | 153.679 | 1.00 | 148.70 |
| 2036 | CD | GLU | B | 390 | 49.982 | 107.940 | 154.417 | 1.00 | 168.33 |
| 2037 | OE1 | GLU | B | 390 | 50.020 | 108.020 | 155.666 | 1.00 | 162.95 |
| 2038 | OE2 | GLU | B | 390 | 50.866 | 107.358 | 153.747 | 1.00 | 166.90 |
| 2039 | C | GLU | B | 390 | 45.000 | 108.231 | 154.301 | 1.00 | 24.57 |
| 2040 | O | GLU | B | 390 | 44.928 | 108.435 | 155.504 | 1.00 | 58.87 |
| 2041 | N | LYS | B | 391 | 44.046 | 107.581 | 153.638 | 1.00 | 39.47 |
| 2042 | CA | LYS | B | 391 | 42.892 | 107.033 | 154.357 | 1.00 | 36.12 |
| 2043 | CB | LYS | B | 391 | 41.939 | 106.319 | 153.392 | 1.00 | 30.68 |
| 2044 | CG | LYS | B | 391 | 40.901 | 105.431 | 154.103 | 1.00 | 91.41 |
| 2045 | CD | LYS | B | 391 | 40.563 | 104.141 | 153.322 | 1.00 | 153.55 |
| 2046 | CE | LYS | B | 391 | 39.785 | 104.403 | 152.029 | 1.00 | 171.40 |
| 2047 | NZ | LYS | B | 391 | 39.360 | 103.133 | 151.353 | 1.00 | 125.88 |
| 2048 | C | LYS | B | 391 | 43.471 | 106.004 | 155.342 | 1.00 | 35.98 |
| 2049 | O | LYS | B | 391 | 44.138 | 105.064 | 154.925 | 1.00 | 52.90 |
| 2050 | N | GLN | B | 392 | 43.232 | 106.173 | 156.636 | 1.00 | 47.33 |
| 2051 | CA | GLN | B | 392 | 43.778 | 105.248 | 157.617 | 1.00 | 50.54 |
| 2052 | CB | GLN | B | 392 | 44.026 | 105.955 | 158.932 | 1.00 | 52.52 |
| 2053 | CG | GLN | B | 392 | 44.910 | 107.158 | 158.819 | 1.00 | 43.19 |
| 2054 | CD | GLN | B | 392 | 46.307 | 106.799 | 158.433 | 1.00 | 52.17 |
| 2055 | OE1 | GLN | B | 392 | 46.549 | 106.342 | 157.315 | 1.00 | 140.45 |
| 2056 | NE2 | GLN | B | 392 | 47.251 | 106.987 | 159.354 | 1.00 | 104.37 |
| 2057 | C | GLN | B | 392 | 42.876 | 104.062 | 157.857 | 1.00 | 83.18 |
| 2058 | O | GLN | B | 392 | 41.730 | 104.057 | 157.421 | 1.00 | 48.10 |
| 2059 | N | ARG | B | 393 | 43.396 | 103.069 | 158.573 | 1.00 | 86.36 |
| 2060 | CA | ARG | B | 393 | 42.646 | 101.853 | 158.851 | 1.00 | 95.47 |
| 2061 | CB | ARG | B | 393 | 43.537 | 100.804 | 159.528 | 1.00 | 129.48 |
| 2062 | CG | ARG | B | 393 | 42.798 | 99.515 | 159.903 | 1.00 | 156.47 |
| 2063 | CD | ARG | B | 393 | 43.235 | 98.309 | 159.074 | 1.00 | 159.01 |
| 2064 | NE | ARG | B | 393 | 44.594 | 97.884 | 159.395 | 1.00 | 164.37 |
| 2065 | CZ | ARG | B | 393 | 45.164 | 96.782 | 158.918 | 1.00 | 170.49 |
| 2066 | NH1 | ARG | B | 393 | 44.492 | 95.988 | 158.094 | 1.00 | 168.61 |
| 2067 | NH2 | ARG | B | 393 | 46.408 | 96.474 | 159.267 | 1.00 | 175.65 |
| 2068 | C | ARG | B | 393 | 41.421 | 102.083 | 159.704 | 1.00 | 67.02 |
| 2069 | O | ARG | B | 393 | 40.379 | 101.483 | 159.461 | 1.00 | 85.39 |
| 2070 | N | ASN | B | 394 | 41.532 | 102.952 | 160.702 | 1.00 | 92.32 |
| 2071 | CA | ASN | B | 394 | 40.400 | 103.198 | 161.591 | 1.00 | 86.89 |
| 2072 | CB | ASN | B | 394 | 40.867 | 103.749 | 162.953 | 1.00 | 80.23 |
| 2073 | CG | ASN | B | 394 | 41.534 | 105.117 | 162.866 | 1.00 | 40.68 |
| 2074 | OD1 | ASN | B | 394 | 41.174 | 105.955 | 162.053 | 1.00 | 63.47 |
| 2075 | ND2 | ASN | B | 394 | 42.490 | 105.336 | 163.761 | 1.00 | 63.72 |
| 2076 | C | ASN | B | 394 | 39.286 | 104.073 | 161.044 | 1.00 | 37.93 |
| 2077 | O | ASN | B | 394 | 38.610 | 104.750 | 161.788 | 1.00 | 46.38 |
| 2078 | N | GLY | B | 395 | 39.075 | 104.048 | 159.740 | 1.00 | 70.31 |
| 2079 | CA | GLY | B | 395 | 38.004 | 104.853 | 159.177 | 1.00 | 65.82 |
| 2080 | C | GLY | B | 395 | 38.289 | 106.338 | 159.044 | 1.00 | 31.15 |
| 2081 | O | GLY | B | 395 | 37.619 | 107.040 | 158.285 | 1.00 | 51.28 |
| 2082 | N | THR | B | 396 | 39.286 | 106.822 | 159.774 | 1.00 | 53.28 |
| 2083 | CA | THR | B | 396 | 39.642 | 108.226 | 159.712 | 1.00 | 40.78 |
| 2084 | CB | THR | B | 396 | 40.600 | 108.599 | 160.829 | 1.00 | 19.53 |
| 2085 | OG1 | THR | B | 396 | 40.696 | 110.023 | 160.904 | 1.00 | 154.97 |
| 2086 | CG2 | THR | B | 396 | 41.976 | 108.064 | 160.557 | 1.00 | 109.19 |
| 2087 | C | THR | B | 396 | 40.296 | 108.573 | 158.383 | 1.00 | 40.28 |
| 2088 | O | THR | B | 396 | 40.205 | 107.818 | 157.422 | 1.00 | 73.66 |
| 2089 | N | LEU | B | 397 | 40.965 | 109.719 | 158.342 | 1.00 | 34.73 |
| 2090 | CA | LEU | B | 397 | 41.643 | 110.210 | 157.146 | 1.00 | 43.25 |
| 2091 | CB | LEU | B | 397 | 40.643 | 110.871 | 156.211 | 1.00 | 57.03 |
| 2092 | CG | LEU | B | 397 | 41.194 | 111.797 | 155.133 | 1.00 | 45.14 |
| 2093 | CD1 | LEU | B | 397 | 42.136 | 111.032 | 154.228 | 1.00 | 110.61 |
| 2094 | CD2 | LEU | B | 397 | 40.032 | 112.353 | 154.322 | 1.00 | 106.87 |
| 2095 | C | LEU | B | 397 | 42.678 | 111.231 | 157.579 | 1.00 | 52.48 |
| 2096 | O | LEU | B | 397 | 42.351 | 112.181 | 158.271 | 1.00 | 67.73 |
| 2097 | N | THR | B | 398 | 43.925 | 111.027 | 157.166 | 1.00 | 62.48 |
| 2098 | CA | THR | B | 398 | 45.033 | 111.910 | 157.522 | 1.00 | 36.96 |
| 2099 | CB | THR | B | 398 | 46.226 | 111.106 | 158.025 | 1.00 | 17.60 |
| 2100 | OG1 | THR | B | 398 | 45.893 | 110.477 | 159.258 | 1.00 | 49.11 |
| 2101 | CG2 | THR | B | 398 | 47.409 | 111.998 | 158.248 | 1.00 | 64.17 |
| 2102 | C | THR | B | 398 | 45.524 | 112.775 | 156.373 | 1.00 | 32.07 |
| 2103 | O | THR | B | 398 | 45.349 | 112.449 | 155.208 | 1.00 | 60.40 |
| 2104 | N | VAL | B | 399 | 46.156 | 113.882 | 156.725 | 1.00 | 24.42 |
| 2105 | CA | VAL | B | 399 | 46.695 | 114.800 | 155.749 | 1.00 | 35.05 |
| 2106 | CB | VAL | B | 399 | 45.788 | 116.004 | 155.569 | 1.00 | 18.51 |
| 2107 | CG1 | VAL | B | 399 | 46.501 | 117.089 | 154.798 | 1.00 | 27.22 |
| 2108 | CG2 | VAL | B | 399 | 44.534 | 115.572 | 154.853 | 1.00 | 29.70 |
| 2109 | C | VAL | B | 399 | 48.011 | 115.300 | 156.268 | 1.00 | 43.68 |
| 2110 | O | VAL | B | 399 | 48.063 | 115.809 | 157.380 | 1.00 | 42.74 |
| 2111 | N | THR | B | 400 | 49.082 | 115.139 | 155.495 | 1.00 | 53.46 |
| 2112 | CA | THR | B | 400 | 50.377 | 115.663 | 155.927 | 1.00 | 43.16 |
| 2113 | CB | THR | B | 400 | 51.450 | 114.598 | 156.085 | 1.00 | 42.86 |
| 2114 | OG1 | THR | B | 400 | 51.697 | 114.011 | 154.813 | 1.00 | 69.60 |
| 2115 | CG2 | THR | B | 400 | 51.014 | 113.532 | 157.036 | 1.00 | 42.99 |
| 2116 | C | THR | B | 400 | 50.862 | 116.598 | 154.844 | 1.00 | 51.26 |
| 2117 | O | THR | B | 400 | 50.595 | 116.382 | 153.656 | 1.00 | 66.11 |
| 2118 | N | SER | B | 401 | 51.573 | 117.637 | 155.261 | 1.00 | 47.73 |
| 2119 | CA | SER | B | 401 | 52.117 | 118.610 | 154.333 | 1.00 | 44.60 |
| 2120 | CB | SER | B | 401 | 51.199 | 119.817 | 154.209 | 1.00 | 50.71 |
| 2121 | OG | SER | B | 401 | 51.810 | 120.856 | 153.475 | 1.00 | 40.79 |
| 2122 | C | SER | B | 401 | 53.457 | 119.052 | 154.862 | 1.00 | 58.39 |
| 2123 | O | SER | B | 401 | 53.551 | 119.546 | 155.993 | 1.00 | 37.36 |
| 2124 | N | THR | B | 402 | 54.495 | 118.859 | 154.048 | 1.00 | 77.89 |
| 2125 | CA | THR | B | 402 | 55.840 | 119.241 | 154.442 | 1.00 | 61.20 |
| 2126 | CB | THR | B | 402 | 56.821 | 118.125 | 154.152 | 1.00 | 61.85 |
| 2127 | OG1 | THR | B | 402 | 56.295 | 116.895 | 154.661 | 1.00 | 81.37 |
| 2128 | CG2 | THR | B | 402 | 58.157 | 118.409 | 154.814 | 1.00 | 109.89 |
| 2129 | C | THR | B | 402 | 56.242 | 120.481 | 153.672 | 1.00 | 68.43 |
| 2130 | O | THR | B | 402 | 55.879 | 120.648 | 152.496 | 1.00 | 56.79 |
| 2131 | N | LEU | B | 403 | 56.991 | 121.354 | 154.339 | 1.00 | 62.35 |
| 2132 | CA | LEU | B | 403 | 57.413 | 122.599 | 153.719 | 1.00 | 65.76 |
| 2133 | CB | LEU | B | 403 | 56.613 | 123.752 | 154.320 | 1.00 | 66.45 |
| 2134 | CG | LEU | B | 403 | 56.909 | 125.152 | 153.790 | 1.00 | 75.07 |
| 2135 | CD1 | LEU | B | 403 | 56.506 | 125.263 | 152.324 | 1.00 | 107.93 |
| 2136 | CD2 | LEU | B | 403 | 56.167 | 126.156 | 154.629 | 1.00 | 75.06 |
| 2137 | C | LEU | B | 403 | 58.912 | 122.897 | 153.838 | 1.00 | 92.32 |
| 2138 | O | LEU | B | 403 | 59.488 | 122.821 | 154.932 | 1.00 | 34.92 |
| 2139 | N | PRO | B | 404 | 59.556 | 123.238 | 152.702 | 1.00 | 49.08 |
| 2140 | CD | PRO | B | 404 | 58.975 | 123.180 | 151.351 | 1.00 | 61.61 |
| 2141 | CA | PRO | B | 404 | 60.976 | 123.560 | 152.621 | 1.00 | 52.97 |
| 2142 | CB | PRO | B | 404 | 61.220 | 123.656 | 151.121 | 1.00 | 111.63 |
| 2143 | CG | PRO | B | 404 | 60.157 | 122.787 | 150.539 | 1.00 | 84.92 |
| 2144 | C | PRO | B | 404 | 61.217 | 124.891 | 153.310 | 1.00 | 73.53 |
| 2145 | O | PRO | B | 404 | 60.473 | 125.843 | 153.112 | 1.00 | 63.87 |
| 2146 | N | VAL | B | 405 | 62.282 | 124.975 | 154.087 | 1.00 | 86.35 |
| 2147 | CA | VAL | B | 405 | 62.543 | 126.198 | 154.812 | 1.00 | 76.26 |
| 2148 | CB | VAL | B | 405 | 62.100 | 126.000 | 156.269 | 1.00 | 36.70 |
| 2149 | CG1 | VAL | B | 405 | 63.203 | 126.416 | 157.230 | 1.00 | 127.12 |
| 2150 | CG2 | VAL | B | 405 | 60.845 | 126.775 | 156.513 | 1.00 | 88.70 |
| 2151 | C | VAL | B | 405 | 63.982 | 126.712 | 154.754 | 1.00 | 107.63 |
| 2152 | O | VAL | B | 405 | 64.940 | 125.951 | 154.939 | 1.00 | 91.98 |
| 2153 | N | GLY | B | 406 | 64.113 | 128.017 | 154.509 | 1.00 | 73.83 |
| 2154 | CA | GLY | B | 406 | 65.427 | 128.645 | 154.435 | 1.00 | 131.05 |
| 2155 | C | GLY | B | 406 | 66.225 | 128.509 | 155.736 | 1.00 | 130.39 |
| 2156 | O | GLY | B | 406 | 65.896 | 129.124 | 156.744 | 1.00 | 100.56 |
| 2157 | N | THR | B | 407 | 67.292 | 127.716 | 155.688 | 1.00 | 142.82 |
| 2158 | CA | THR | B | 407 | 68.143 | 127.461 | 156.830 | 1.00 | 100.01 |
| 2159 | CB | THR | B | 407 | 69.482 | 126.859 | 156.428 | 1.00 | 126.91 |
| 2160 | OG1 | THR | B | 407 | 69.350 | 126.170 | 155.184 | 1.00 | 151.73 |
| 2161 | CG2 | THR | B | 407 | 69.964 | 125.900 | 157.521 | 1.00 | 85.87 |
| 2162 | C | THR | B | 407 | 68.488 | 128.714 | 157.631 | 1.00 | 111.52 |
| 2163 | O | THR | B | 407 | 68.563 | 128.679 | 158.858 | 1.00 | 92.93 |
| 2164 | N | ARG | B | 408 | 68.734 | 129.816 | 156.934 | 1.00 | 135.67 |
| 2165 | CA | ARG | B | 408 | 69.098 | 131.063 | 157.590 | 1.00 | 137.76 |
| 2166 | CB | ARG | B | 408 | 69.517 | 132.097 | 156.546 | 1.00 | 153.22 |
| 2167 | CG | ARG | B | 408 | 70.749 | 131.688 | 155.715 | 1.00 | 174.72 |
| 2168 | CD | ARG | B | 408 | 70.530 | 130.402 | 154.906 | 1.00 | 170.05 |
| 2169 | NE | ARG | B | 408 | 69.575 | 130.568 | 153.813 | 1.00 | 157.68 |
| 2170 | CZ | ARG | B | 408 | 69.164 | 129.575 | 153.031 | 1.00 | 144.79 |
| 2171 | NH1 | ARG | B | 408 | 69.617 | 128.342 | 153.226 | 1.00 | 99.09 |
| 2172 | NH2 | ARG | B | 408 | 68.314 | 129.818 | 152.043 | 1.00 | 134.60 |
| 2173 | C | ARG | B | 408 | 67.905 | 131.589 | 158.374 | 1.00 | 125.74 |
| 2174 | O | ARG | B | 408 | 67.885 | 131.590 | 159.622 | 1.00 | 107.88 |
| 2175 | N | ASP | B | 409 | 66.897 | 132.018 | 157.626 | 1.00 | 114.38 |
| 2176 | CA | ASP | B | 409 | 65.665 | 132.577 | 158.171 | 1.00 | 100.02 |
| 2177 | CB | ASP | B | 409 | 64.547 | 132.393 | 157.129 | 1.00 | 106.65 |
| 2178 | CG | ASP | B | 409 | 64.925 | 132.915 | 155.743 | 1.00 | 131.77 |
| 2179 | OD1 | ASP | B | 409 | 64.961 | 134.151 | 155.545 | 1.00 | 128.42 |
| 2180 | OD2 | ASP | B | 409 | 65.187 | 132.086 | 154.841 | 1.00 | 107.93 |
| 2181 | C | ASP | B | 409 | 65.242 | 131.954 | 159.503 | 1.00 | 87.73 |
| 2182 | O | ASP | B | 409 | 64.932 | 132.667 | 160.459 | 1.00 | 125.50 |
| 2183 | N | TRP | B | 410 | 65.229 | 130.626 | 159.553 | 1.00 | 82.03 |
| 2184 | CA | TRP | B | 410 | 64.836 | 129.888 | 160.749 | 1.00 | 68.60 |
| 2185 | CB | TRP | B | 410 | 64.923 | 128.385 | 160.486 | 1.00 | 79.72 |
| 2186 | CG | TRP | B | 410 | 64.743 | 127.540 | 161.706 | 1.00 | 47.85 |
| 2187 | CD2 | TRP | B | 410 | 63.509 | 127.028 | 162.201 | 1.00 | 87.88 |
| 2188 | CE2 | TRP | B | 410 | 63.796 | 126.300 | 163.380 | 1.00 | 73.32 |
| 2189 | CE3 | TRP | B | 410 | 62.184 | 127.117 | 161.764 | 1.00 | 50.59 |
| 2190 | CD1 | TRP | B | 410 | 65.711 | 127.116 | 162.577 | 1.00 | 100.57 |
| 2191 | NE1 | TRP | B | 410 | 65.147 | 126.367 | 163.587 | 1.00 | 53.96 |
| 2192 | CZ2 | TRP | B | 410 | 62.806 | 125.668 | 164.127 | 1.00 | 90.94 |
| 2193 | CZ3 | TRP | B | 410 | 61.203 | 126.490 | 162.505 | 1.00 | 42.94 |
| 2194 | CH2 | TRP | B | 410 | 61.517 | 125.773 | 163.678 | 1.00 | 64.03 |
| 2195 | C | TRP | B | 410 | 65.675 | 130.226 | 161.960 | 1.00 | 98.97 |
| 2196 | O | TRP | B | 410 | 65.141 | 130.650 | 162.976 | 1.00 | 129.24 |
| 2197 | N | ILE | B | 411 | 66.985 | 130.031 | 161.860 | 1.00 | 106.60 |
| 2198 | CA | ILE | B | 411 | 67.850 | 130.306 | 162.998 | 1.00 | 115.95 |
| 2199 | CB | ILE | B | 411 | 69.317 | 130.062 | 162.670 | 1.00 | 103.18 |
| 2200 | CG2 | ILE | B | 411 | 70.075 | 129.721 | 163.955 | 1.00 | 112.78 |
| 2201 | CG1 | ILE | B | 411 | 69.438 | 128.905 | 161.683 | 1.00 | 127.25 |
| 2202 | CD1 | ILE | B | 411 | 70.843 | 128.694 | 161.174 | 1.00 | 162.43 |
| 2203 | C | ILE | B | 411 | 67.701 | 131.744 | 163.459 | 1.00 | 130.64 |
| 2204 | O | ILE | B | 411 | 67.892 | 132.055 | 164.637 | 1.00 | 115.79 |
| 2205 | N | GLU | B | 412 | 67.359 | 132.627 | 162.529 | 1.00 | 89.59 |
| 2206 | CA | GLU | B | 412 | 67.183 | 134.023 | 162.883 | 1.00 | 115.46 |
| 2207 | CB | GLU | B | 412 | 67.480 | 134.912 | 161.677 | 1.00 | 137.38 |
| 2208 | CG | GLU | B | 412 | 68.974 | 135.047 | 161.407 | 1.00 | 156.60 |
| 2209 | CD | GLU | B | 412 | 69.283 | 136.016 | 160.288 | 1.00 | 185.08 |
| 2210 | OE1 | GLU | B | 412 | 68.761 | 137.150 | 160.329 | 1.00 | 188.44 |
| 2211 | OE2 | GLU | B | 412 | 70.051 | 135.648 | 159.374 | 1.00 | 188.13 |
| 2212 | C | GLU | B | 412 | 65.799 | 134.316 | 163.458 | 1.00 | 140.17 |
| 2213 | O | GLU | B | 412 | 65.262 | 135.411 | 163.299 | 1.00 | 136.77 |
| 2214 | N | GLY | B | 413 | 65.228 | 133.314 | 164.121 | 1.00 | 164.88 |
| 2215 | CA | GLY | B | 413 | 63.931 | 133.461 | 164.761 | 1.00 | 159.55 |
| 2216 | C | GLY | B | 413 | 62.653 | 133.570 | 163.947 | 1.00 | 139.70 |
| 2217 | O | GLY | B | 413 | 61.592 | 133.808 | 164.523 | 1.00 | 138.95 |
| 2218 | N | GLU | B | 414 | 62.720 | 133.404 | 162.631 | 1.00 | 131.17 |
| 2219 | CA | GLU | B | 414 | 61.509 | 133.499 | 161.824 | 1.00 | 96.50 |
| 2220 | CB | GLU | B | 414 | 61.778 | 133.066 | 160.389 | 1.00 | 107.97 |
| 2221 | CG | GLU | B | 414 | 60.530 | 132.974 | 159.525 | 1.00 | 80.41 |
| 2222 | CD | GLU | B | 414 | 59.820 | 134.291 | 159.380 | 1.00 | 87.05 |
| 2223 | OE1 | GLU | B | 414 | 59.242 | 134.775 | 160.373 | 1.00 | 111.31 |
| 2224 | OE2 | GLU | B | 414 | 59.847 | 134.849 | 158.265 | 1.00 | 93.50 |
| 2225 | C | GLU | B | 414 | 60.420 | 132.622 | 162.423 | 1.00 | 101.40 |
| 2226 | O | GLU | B | 414 | 60.687 | 131.784 | 163.285 | 1.00 | 104.23 |
| 2227 | N | THR | B | 415 | 59.192 | 132.819 | 161.960 | 1.00 | 115.03 |
| 2228 | CA | THR | B | 415 | 58.056 | 132.059 | 162.458 | 1.00 | 112.22 |
| 2229 | CB | THR | B | 415 | 57.348 | 132.843 | 163.575 | 1.00 | 118.03 |
| 2230 | OG1 | THR | B | 415 | 55.994 | 132.403 | 163.687 | 1.00 | 110.64 |
| 2231 | CG2 | THR | B | 415 | 57.422 | 134.334 | 163.313 | 1.00 | 155.73 |
| 2232 | C | THR | B | 415 | 57.055 | 131.660 | 161.366 | 1.00 | 127.06 |
| 2233 | O | THR | B | 415 | 56.517 | 132.506 | 160.626 | 1.00 | 84.52 |
| 2234 | N | TYR | B | 416 | 56.818 | 130.351 | 161.282 | 1.00 | 115.33 |
| 2235 | CA | TYR | B | 416 | 55.922 | 129.765 | 160.287 | 1.00 | 76.98 |
| 2236 | CB | TYR | B | 416 | 56.626 | 128.606 | 159.569 | 1.00 | 86.80 |
| 2237 | CG | TYR | B | 416 | 57.961 | 128.956 | 158.940 | 1.00 | 94.67 |
| 2238 | CD1 | TYR | B | 416 | 59.155 | 128.835 | 159.662 | 1.00 | 110.20 |
| 2239 | CE1 | TYR | B | 416 | 60.380 | 129.198 | 159.099 | 1.00 | 47.43 |
| 2240 | CD2 | TYR | B | 416 | 58.024 | 129.446 | 157.635 | 1.00 | 61.95 |
| 2241 | CE2 | TYR | B | 416 | 59.231 | 129.815 | 157.066 | 1.00 | 64.97 |
| 2242 | CZ | TYR | B | 416 | 60.408 | 129.693 | 157.797 | 1.00 | 90.31 |
| 2243 | OH | TYR | B | 416 | 61.601 | 130.081 | 157.222 | 1.00 | 162.34 |
| 2244 | C | TYR | B | 416 | 54.623 | 129.255 | 160.897 | 1.00 | 92.23 |
| 2245 | O | TYR | B | 416 | 54.604 | 128.778 | 162.041 | 1.00 | 59.60 |
| 2246 | N | GLN | B | 417 | 53.546 | 129.322 | 160.115 | 1.00 | 62.55 |
| 2247 | CA | GLN | B | 417 | 52.236 | 128.889 | 160.592 | 1.00 | 50.69 |
| 2248 | CB | GLN | B | 417 | 51.411 | 130.114 | 160.969 | 1.00 | 129.83 |
| 2249 | CG | GLN | B | 417 | 50.127 | 129.811 | 161.708 | 1.00 | 147.33 |
| 2250 | CD | GLN | B | 417 | 49.282 | 131.051 | 161.924 | 1.00 | 140.76 |
| 2251 | OE1 | GLN | B | 417 | 48.740 | 131.624 | 160.971 | 1.00 | 120.92 |
| 2252 | NE2 | GLN | B | 417 | 49.167 | 131.477 | 163.181 | 1.00 | 129.75 |
| 2253 | C | GLN | B | 417 | 51.457 | 128.058 | 159.575 | 1.00 | 59.75 |
| 2254 | O | GLN | B | 417 | 51.437 | 128.363 | 158.381 | 1.00 | 76.03 |
| 2255 | N | CYS | B | 418 | 50.796 | 127.018 | 160.080 | 1.00 | 69.24 |
| 2256 | CA | CYS | B | 418 | 49.998 | 126.095 | 159.267 | 1.00 | 63.48 |
| 2257 | C | CYS | B | 418 | 48.521 | 126.354 | 159.532 | 1.00 | 71.26 |
| 2258 | O | CYS | B | 418 | 48.083 | 126.288 | 160.688 | 1.00 | 66.93 |
| 2259 | CB | CYS | B | 418 | 50.343 | 124.647 | 159.636 | 1.00 | 83.48 |
| 2260 | SG | CYS | B | 418 | 49.465 | 123.360 | 158.682 | 1.00 | 121.42 |
| 2261 | N | ARG | B | 419 | 47.756 | 126.640 | 158.474 | 1.00 | 40.08 |
| 2262 | CA | ARG | B | 419 | 46.327 | 126.928 | 158.637 | 1.00 | 38.15 |
| 2263 | CB | ARG | B | 419 | 46.008 | 128.369 | 158.211 | 1.00 | 106.47 |
| 2264 | CG | ARG | B | 419 | 44.562 | 128.797 | 158.483 | 1.00 | 184.87 |
| 2265 | CD | ARG | B | 419 | 44.235 | 130.164 | 157.902 | 1.00 | 224.39 |
| 2266 | NE | ARG | B | 419 | 42.880 | 130.587 | 158.248 | 1.00 | 235.46 |
| 2267 | CZ | ARG | B | 419 | 42.299 | 131.685 | 157.782 | 1.00 | 221.27 |
| 2268 | NH1 | ARG | B | 419 | 42.952 | 132.475 | 156.945 | 1.00 | 212.86 |
| 2269 | NH2 | ARG | B | 419 | 41.067 | 131.995 | 158.156 | 1.00 | 204.55 |
| 2270 | C | ARG | B | 419 | 45.434 | 125.975 | 157.868 | 1.00 | 66.29 |
| 2271 | O | ARG | B | 419 | 45.318 | 126.081 | 156.658 | 1.00 | 49.15 |
| 2272 | N | VAL | B | 420 | 44.777 | 125.056 | 158.565 | 1.00 | 41.47 |
| 2273 | CA | VAL | B | 420 | 43.891 | 124.107 | 157.877 | 1.00 | 57.57 |
| 2274 | CB | VAL | B | 420 | 44.050 | 122.684 | 158.466 | 1.00 | 53.54 |
| 2275 | CG1 | VAL | B | 420 | 45.434 | 122.539 | 159.084 | 1.00 | 58.35 |
| 2276 | CG2 | VAL | B | 420 | 42.980 | 122.416 | 159.517 | 1.00 | 71.65 |
| 2277 | C | VAL | B | 420 | 42.439 | 124.602 | 158.000 | 1.00 | 50.86 |
| 2278 | O | VAL | B | 420 | 42.085 | 125.213 | 159.020 | 1.00 | 51.31 |
| 2279 | N | THR | B | 421 | 41.636 | 124.307 | 156.966 | 1.00 | 31.63 |
| 2280 | CA | THR | B | 421 | 40.259 | 124.752 | 156.844 | 1.00 | 41.77 |
| 2281 | CB | THR | B | 421 | 40.214 | 126.119 | 156.170 | 1.00 | 12.47 |
| 2282 | OG1 | THR | B | 421 | 40.364 | 127.127 | 157.168 | 1.00 | 107.15 |
| 2283 | CG2 | THR | B | 421 | 38.907 | 126.314 | 155.393 | 1.00 | 70.23 |
| 2284 | C | THR | B | 421 | 39.294 | 123.902 | 156.029 | 1.00 | 49.50 |
| 2285 | O | THR | B | 421 | 39.438 | 123.788 | 154.818 | 1.00 | 60.59 |
| 2286 | N | HIS | B | 422 | 38.259 | 123.357 | 156.640 | 1.00 | 61.87 |
| 2287 | CA | HIS | B | 422 | 37.333 | 122.547 | 155.856 | 1.00 | 46.82 |
| 2288 | CB | HIS | B | 422 | 37.688 | 121.069 | 156.046 | 1.00 | 54.88 |
| 2289 | CG | HIS | B | 422 | 36.899 | 120.137 | 155.186 | 1.00 | 66.28 |
| 2290 | CD2 | HIS | B | 422 | 36.786 | 120.023 | 153.852 | 1.00 | 90.39 |
| 2291 | ND1 | HIS | B | 422 | 36.142 | 119.123 | 155.730 | 1.00 | 105.82 |
| 2292 | CE1 | HIS | B | 422 | 35.595 | 118.426 | 154.754 | 1.00 | 67.94 |
| 2293 | NE2 | HIS | B | 422 | 35.968 | 118.950 | 153.598 | 1.00 | 89.25 |
| 2294 | C | HIS | B | 422 | 35.883 | 122.789 | 156.278 | 1.00 | 35.50 |
| 2295 | O | HIS | B | 422 | 35.615 | 123.100 | 157.427 | 1.00 | 63.71 |
| 2296 | N | PRO | B | 423 | 34.938 | 122.661 | 155.333 | 1.00 | 33.86 |
| 2297 | CD | PRO | B | 423 | 35.231 | 122.574 | 153.889 | 1.00 | 61.95 |
| 2298 | CA | PRO | B | 423 | 33.500 | 122.844 | 155.563 | 1.00 | 74.49 |
| 2299 | CB | PRO | B | 423 | 32.892 | 122.227 | 154.320 | 1.00 | 30.75 |
| 2300 | CG | PRO | B | 423 | 33.833 | 122.723 | 153.266 | 1.00 | 92.20 |
| 2301 | C | PRO | B | 423 | 33.024 | 122.170 | 156.843 | 1.00 | 25.99 |
| 2302 | O | PRO | B | 423 | 32.254 | 122.745 | 157.604 | 1.00 | 94.74 |
| 2303 | N | HIS | B | 424 | 33.489 | 120.951 | 157.078 | 1.00 | 72.49 |
| 2304 | CA | HIS | B | 424 | 33.139 | 120.178 | 158.262 | 1.00 | 35.64 |
| 2305 | CB | HIS | B | 424 | 33.886 | 118.850 | 158.263 | 1.00 | 68.85 |
| 2306 | CG | HIS | B | 424 | 33.378 | 117.865 | 157.264 | 1.00 | 33.77 |
| 2307 | CD2 | HIS | B | 424 | 32.613 | 118.022 | 156.163 | 1.00 | 93.93 |
| 2308 | ND1 | HIS | B | 424 | 33.632 | 116.516 | 157.368 | 1.00 | 22.18 |
| 2309 | CE1 | HIS | B | 424 | 33.043 | 115.883 | 156.373 | 1.00 | 72.12 |
| 2310 | NE2 | HIS | B | 424 | 32.417 | 116.774 | 155.626 | 1.00 | 92.83 |
| 2311 | C | HIS | B | 424 | 33.562 | 120.919 | 159.503 | 1.00 | 72.43 |
| 2312 | O | HIS | B | 424 | 33.155 | 120.583 | 160.614 | 1.00 | 45.64 |
| 2313 | N | LEU | B | 425 | 34.397 | 121.925 | 159.310 | 1.00 | 30.28 |
| 2314 | CA | LEU | B | 425 | 34.907 | 122.679 | 160.429 | 1.00 | 67.62 |
| 2315 | CB | LEU | B | 425 | 36.415 | 122.774 | 160.284 | 1.00 | 51.81 |
| 2316 | CG | LEU | B | 425 | 37.061 | 121.393 | 160.189 | 1.00 | 50.51 |
| 2317 | CD1 | LEU | B | 425 | 38.563 | 121.538 | 160.155 | 1.00 | 99.40 |
| 2318 | CD2 | LEU | B | 425 | 36.651 | 120.562 | 161.377 | 1.00 | 41.28 |
| 2319 | C | LEU | B | 425 | 34.323 | 124.059 | 160.708 | 1.00 | 63.27 |
| 2320 | O | LEU | B | 425 | 34.105 | 124.859 | 159.800 | 1.00 | 102.78 |
| 2321 | N | PRO | B | 426 | 34.071 | 124.348 | 161.994 | 1.00 | 57.60 |
| 2322 | CD | PRO | B | 426 | 34.288 | 123.365 | 163.068 | 1.00 | 65.15 |
| 2323 | CA | PRO | B | 426 | 33.523 | 125.589 | 162.550 | 1.00 | 52.46 |
| 2324 | CB | PRO | B | 426 | 33.167 | 125.178 | 163.965 | 1.00 | 85.27 |
| 2325 | CG | PRO | B | 426 | 34.270 | 124.230 | 164.292 | 1.00 | 34.92 |
| 2326 | C | PRO | B | 426 | 34.576 | 126.710 | 162.531 | 1.00 | 49.56 |
| 2327 | O | PRO | B | 426 | 34.524 | 127.605 | 161.692 | 1.00 | 85.61 |
| 2328 | N | ARG | B | 427 | 35.513 | 126.657 | 163.475 | 1.00 | 56.31 |
| 2329 | CA | ARG | B | 427 | 36.606 | 127.626 | 163.550 | 1.00 | 81.37 |
| 2330 | CB | ARG | B | 427 | 37.101 | 127.788 | 165.016 | 1.00 | 37.35 |
| 2331 | CG | ARG | B | 427 | 37.494 | 126.478 | 165.701 | 1.00 | 67.67 |
| 2332 | CD | ARG | B | 427 | 37.021 | 126.334 | 167.163 | 1.00 | 106.61 |
| 2333 | NE | ARG | B | 427 | 36.476 | 124.989 | 167.420 | 1.00 | 156.12 |
| 2334 | CZ | ARG | B | 427 | 36.257 | 124.464 | 168.627 | 1.00 | 157.58 |
| 2335 | NH1 | ARG | B | 427 | 36.543 | 125.167 | 169.717 | 1.00 | 155.60 |
| 2336 | NH2 | ARG | B | 427 | 35.737 | 123.236 | 168.748 | 1.00 | 66.93 |
| 2337 | C | ARG | B | 427 | 37.709 | 127.037 | 162.664 | 1.00 | 71.40 |
| 2338 | O | ARG | B | 427 | 37.641 | 125.876 | 162.279 | 1.00 | 38.48 |
| 2339 | N | ALA | B | 428 | 38.713 | 127.824 | 162.313 | 1.00 | 105.95 |
| 2340 | CA | ALA | B | 428 | 39.793 | 127.289 | 161.495 | 1.00 | 47.56 |
| 2341 | CB | ALA | B | 428 | 40.458 | 128.403 | 160.734 | 1.00 | 123.77 |
| 2342 | C | ALA | B | 428 | 40.782 | 126.651 | 162.451 | 1.00 | 60.43 |
| 2343 | O | ALA | B | 428 | 40.721 | 126.891 | 163.651 | 1.00 | 68.94 |
| 2344 | N | LEU | B | 429 | 41.689 | 125.836 | 161.932 | 1.00 | 64.23 |
| 2345 | CA | LEU | B | 429 | 42.698 | 125.194 | 162.769 | 1.00 | 63.17 |
| 2346 | CB | LEU | B | 429 | 42.750 | 123.697 | 162.523 | 1.00 | 33.26 |
| 2347 | CG | LEU | B | 429 | 42.661 | 122.872 | 163.803 | 1.00 | 88.80 |
| 2348 | CD1 | LEU | B | 429 | 42.937 | 121.426 | 163.444 | 1.00 | 60.45 |
| 2349 | CD2 | LEU | B | 429 | 43.645 | 123.380 | 164.857 | 1.00 | 119.69 |
| 2350 | C | LEU | B | 429 | 44.041 | 125.779 | 162.418 | 1.00 | 60.60 |
| 2351 | O | LEU | B | 429 | 44.392 | 125.847 | 161.247 | 1.00 | 46.41 |
| 2352 | N | MET | B | 430 | 44.792 | 126.186 | 163.433 | 1.00 | 45.54 |
| 2353 | CA | MET | B | 430 | 46.085 | 126.789 | 163.209 | 1.00 | 42.39 |
| 2354 | CB | MET | B | 430 | 46.004 | 128.321 | 163.336 | 1.00 | 51.46 |
| 2355 | CG | MET | B | 430 | 45.156 | 129.011 | 162.277 | 1.00 | 72.95 |
| 2356 | SD | MET | B | 430 | 45.247 | 130.811 | 162.351 | 1.00 | 135.30 |
| 2357 | CE | MET | B | 430 | 44.061 | 131.159 | 163.665 | 1.00 | 161.36 |
| 2358 | C | MET | B | 430 | 47.063 | 126.286 | 164.226 | 1.00 | 61.09 |
| 2359 | O | MET | B | 430 | 46.686 | 125.890 | 165.335 | 1.00 | 52.58 |
| 2360 | N | ARG | B | 431 | 48.326 | 126.285 | 163.821 | 1.00 | 42.23 |
| 2361 | CA | ARG | B | 431 | 49.423 | 125.906 | 164.696 | 1.00 | 63.07 |
| 2362 | CB | ARG | B | 431 | 49.602 | 124.404 | 164.736 | 1.00 | 33.42 |
| 2363 | CG | ARG | B | 431 | 48.302 | 123.660 | 164.986 | 1.00 | 57.91 |
| 2364 | CD | ARG | B | 431 | 48.510 | 122.413 | 165.816 | 1.00 | 51.62 |
| 2365 | NE | ARG | B | 431 | 48.125 | 122.644 | 167.201 | 1.00 | 93.78 |
| 2366 | CZ | ARG | B | 431 | 46.876 | 122.871 | 167.585 | 1.00 | 101.45 |
| 2367 | NH1 | ARG | B | 431 | 45.907 | 122.888 | 166.681 | 1.00 | 36.13 |
| 2368 | NH2 | ARG | B | 431 | 46.601 | 123.088 | 168.865 | 1.00 | 161.49 |
| 2369 | C | ARG | B | 431 | 50.627 | 126.588 | 164.085 | 1.00 | 72.59 |
| 2370 | O | ARG | B | 431 | 50.663 | 126.820 | 162.869 | 1.00 | 53.93 |
| 2371 | N | SER | B | 432 | 51.589 | 126.941 | 164.928 | 1.00 | 70.72 |
| 2372 | CA | SER | B | 432 | 52.772 | 127.634 | 164.457 | 1.00 | 71.54 |
| 2373 | CB | SER | B | 432 | 52.625 | 129.138 | 164.671 | 1.00 | 72.40 |
| 2374 | OG | SER | B | 432 | 52.566 | 129.443 | 166.058 | 1.00 | 106.30 |
| 2375 | C | SER | B | 432 | 53.974 | 127.142 | 165.224 | 1.00 | 99.13 |
| 2376 | O | SER | B | 432 | 53.831 | 126.433 | 166.226 | 1.00 | 55.86 |
| 2377 | N | THR | B | 433 | 55.155 | 127.529 | 164.749 | 1.00 | 84.30 |
| 2378 | CA | THR | B | 433 | 56.400 | 127.130 | 165.384 | 1.00 | 88.35 |
| 2379 | CB | THR | B | 433 | 56.722 | 125.683 | 165.096 | 1.00 | 76.49 |
| 2380 | OG1 | THR | B | 433 | 57.898 | 125.303 | 165.820 | 1.00 | 105.68 |
| 2381 | CG2 | THR | B | 433 | 56.953 | 125.506 | 163.607 | 1.00 | 49.60 |
| 2382 | C | THR | B | 433 | 57.567 | 127.947 | 164.891 | 1.00 | 97.22 |
| 2383 | O | THR | B | 433 | 57.576 | 128.410 | 163.734 | 1.00 | 66.32 |
| 2384 | N | THR | B | 434 | 58.546 | 128.111 | 165.785 | 1.00 | 77.69 |
| 2385 | CA | THR | B | 434 | 59.786 | 128.838 | 165.511 | 1.00 | 65.57 |
| 2386 | CB | THR | B | 434 | 59.687 | 130.335 | 165.820 | 1.00 | 84.37 |
| 2387 | OG1 | THR | B | 434 | 59.875 | 130.555 | 167.229 | 1.00 | 140.54 |
| 2388 | CG2 | THR | B | 434 | 58.342 | 130.886 | 165.421 | 1.00 | 65.31 |
| 2389 | C | THR | B | 434 | 60.880 | 128.307 | 166.436 | 1.00 | 96.20 |
| 2390 | O | THR | B | 434 | 60.621 | 127.548 | 167.360 | 1.00 | 67.43 |
| 2391 | N | LYS | B | 435 | 62.095 | 128.750 | 166.146 | 1.00 | 102.83 |
| 2392 | CA | LYS | B | 435 | 63.296 | 128.401 | 166.859 | 1.00 | 94.54 |
| 2393 | CB | LYS | B | 435 | 64.392 | 129.385 | 166.468 | 1.00 | 102.66 |
| 2394 | CG | LYS | B | 435 | 65.736 | 129.328 | 167.181 | 1.00 | 156.34 |
| 2395 | CD | LYS | B | 435 | 66.662 | 130.374 | 166.492 | 1.00 | 155.89 |
| 2396 | CE | LYS | B | 435 | 68.100 | 130.543 | 167.040 | 1.00 | 174.55 |
| 2397 | NZ | LYS | B | 435 | 68.097 | 131.005 | 168.483 | 1.00 | 180.88 |
| 2398 | C | LYS | B | 435 | 63.073 | 128.443 | 168.369 | 1.00 | 90.68 |
| 2399 | O | LYS | B | 435 | 62.554 | 129.421 | 168.898 | 1.00 | 125.40 |
| 2400 | N | THR | B | 436 | 63.449 | 127.383 | 169.070 | 1.00 | 65.76 |
| 2401 | CA | THR | B | 436 | 63.293 | 127.351 | 170.526 | 1.00 | 80.29 |
| 2402 | CB | THR | B | 436 | 63.710 | 126.002 | 171.078 | 1.00 | 74.52 |
| 2403 | OG1 | THR | B | 436 | 63.054 | 124.968 | 170.344 | 1.00 | 133.68 |
| 2404 | CG2 | THR | B | 436 | 63.341 | 125.879 | 172.532 | 1.00 | 84.46 |
| 2405 | C | THR | B | 436 | 64.159 | 128.429 | 171.186 | 1.00 | 132.09 |
| 2406 | O | THR | B | 436 | 65.294 | 128.670 | 170.764 | 1.00 | 135.39 |
| 2407 | N | SER | B | 437 | 63.639 | 129.031 | 172.255 | 1.00 | 135.64 |
| 2408 | CA | SER | B | 437 | 64.341 | 130.112 | 172.957 | 1.00 | 154.94 |
| 2409 | CB | SER | B | 437 | 63.334 | 131.187 | 173.370 | 1.00 | 159.96 |
| 2410 | OG | SER | B | 437 | 62.326 | 130.634 | 174.204 | 1.00 | 151.19 |
| 2411 | C | SER | B | 437 | 65.157 | 129.701 | 174.176 | 1.00 | 145.96 |
| 2412 | O | SER | B | 437 | 65.253 | 128.518 | 174.505 | 1.00 | 126.88 |
| 2413 | N | GLY | B | 438 | 65.748 | 130.698 | 174.834 | 1.00 | 142.79 |
| 2414 | CA | GLY | B | 438 | 66.544 | 130.446 | 176.022 | 1.00 | 127.41 |
| 2415 | C | GLY | B | 438 | 68.046 | 130.551 | 175.823 | 1.00 | 105.05 |
| 2416 | O | GLY | B | 438 | 68.511 | 131.074 | 174.814 | 1.00 | 92.39 |
| 2417 | N | PRO | B | 439 | 68.836 | 130.081 | 176.796 | 1.00 | 96.54 |
| 2418 | CD | PRO | B | 439 | 68.372 | 129.693 | 178.137 | 1.00 | 102.85 |
| 2419 | CA | PRO | B | 439 | 70.299 | 130.103 | 176.748 | 1.00 | 119.07 |
| 2420 | CB | PRO | B | 439 | 70.684 | 130.075 | 178.218 | 1.00 | 146.55 |
| 2421 | CG | PRO | B | 439 | 69.637 | 129.177 | 178.787 | 1.00 | 118.33 |
| 2422 | C | PRO | B | 439 | 70.807 | 128.876 | 175.989 | 1.00 | 126.07 |
| 2423 | O | PRO | B | 439 | 70.310 | 127.768 | 176.199 | 1.00 | 134.45 |
| 2424 | N | ARG | B | 440 | 71.805 | 129.094 | 175.127 | 1.00 | 120.46 |
| 2425 | CA | ARG | B | 440 | 72.374 | 128.039 | 174.291 | 1.00 | 111.19 |
| 2426 | CB | ARG | B | 440 | 72.576 | 128.565 | 172.866 | 1.00 | 122.47 |
| 2427 | CG | ARG | B | 440 | 71.504 | 129.538 | 172.390 | 1.00 | 147.23 |
| 2428 | CD | ARG | B | 440 | 70.181 | 128.851 | 172.101 | 1.00 | 186.68 |
| 2429 | NE | ARG | B | 440 | 69.065 | 129.796 | 172.154 | 1.00 | 214.53 |
| 2430 | CZ | ARG | B | 440 | 67.972 | 129.726 | 171.400 | 1.00 | 221.94 |
| 2431 | NH1 | ARG | B | 440 | 67.829 | 128.750 | 170.511 | 1.00 | 219.20 |
| 2432 | NH2 | ARG | B | 440 | 67.023 | 130.642 | 171.541 | 1.00 | 205.02 |
| 2433 | C | ARG | B | 440 | 73.699 | 127.481 | 174.812 | 1.00 | 89.64 |
| 2434 | O | ARG | B | 440 | 74.753 | 128.090 | 174.628 | 1.00 | 132.22 |
| 2435 | N | ALA | B | 441 | 73.650 | 126.315 | 175.450 | 1.00 | 73.11 |
| 2436 | CA | ALA | B | 441 | 74.869 | 125.692 | 175.965 | 1.00 | 83.71 |
| 2437 | CB | ALA | B | 441 | 74.701 | 125.340 | 177.430 | 1.00 | 94.78 |
| 2438 | C | ALA | B | 441 | 75.217 | 124.441 | 175.151 | 1.00 | 85.35 |
| 2439 | O | ALA | B | 441 | 74.409 | 123.514 | 175.047 | 1.00 | 93.65 |
| 2440 | N | ALA | B | 442 | 76.430 | 124.427 | 174.595 | 1.00 | 115.97 |
| 2441 | CA | ALA | B | 442 | 76.938 | 123.321 | 173.775 | 1.00 | 86.76 |
| 2442 | CB | ALA | B | 442 | 78.341 | 123.654 | 173.274 | 1.00 | 132.70 |
| 2443 | C | ALA | B | 442 | 76.946 | 121.954 | 174.466 | 1.00 | 93.35 |
| 2444 | O | ALA | B | 442 | 76.879 | 121.858 | 175.693 | 1.00 | 88.94 |
| 2445 | N | PRO | B | 443 | 77.045 | 120.877 | 173.670 | 1.00 | 88.73 |
| 2446 | CD | PRO | B | 443 | 76.967 | 120.894 | 172.202 | 1.00 | 53.59 |
| 2447 | CA | PRO | B | 443 | 77.057 | 119.497 | 174.160 | 1.00 | 66.28 |
| 2448 | CB | PRO | B | 443 | 76.504 | 118.696 | 172.982 | 1.00 | 75.52 |
| 2449 | CG | PRO | B | 443 | 76.059 | 119.745 | 171.951 | 1.00 | 82.83 |
| 2450 | C | PRO | B | 443 | 78.413 | 118.970 | 174.561 | 1.00 | 69.00 |
| 2451 | O | PRO | B | 443 | 79.442 | 119.348 | 174.008 | 1.00 | 107.52 |
| 2452 | N | GLU | B | 444 | 78.391 | 118.073 | 175.529 | 1.00 | 79.76 |
| 2453 | CA | GLU | B | 444 | 79.594 | 117.429 | 176.022 | 1.00 | 111.19 |
| 2454 | CB | GLU | B | 444 | 79.710 | 117.589 | 177.542 | 1.00 | 148.56 |
| 2455 | CG | GLU | B | 444 | 80.142 | 118.972 | 178.015 | 1.00 | 175.46 |
| 2456 | CD | GLU | B | 444 | 79.828 | 119.211 | 179.486 | 1.00 | 159.46 |
| 2457 | OE1 | GLU | B | 444 | 79.978 | 118.265 | 180.292 | 1.00 | 128.82 |
| 2458 | OE2 | GLU | B | 444 | 79.438 | 120.349 | 179.836 | 1.00 | 148.10 |
| 2459 | C | GLU | B | 444 | 79.351 | 115.976 | 175.686 | 1.00 | 93.99 |
| 2460 | O | GLU | B | 444 | 78.357 | 115.406 | 176.121 | 1.00 | 102.87 |
| 2461 | N | VAL | B | 445 | 80.235 | 115.375 | 174.903 | 1.00 | 98.05 |
| 2462 | CA | VAL | B | 445 | 80.056 | 113.978 | 174.543 | 1.00 | 82.80 |
| 2463 | CB | VAL | B | 445 | 79.787 | 113.845 | 173.042 | 1.00 | 75.64 |
| 2464 | CG1 | VAL | B | 445 | 80.696 | 114.770 | 172.266 | 1.00 | 56.15 |
| 2465 | CG2 | VAL | B | 445 | 79.983 | 112.403 | 172.620 | 1.00 | 100.09 |
| 2466 | C | VAL | B | 445 | 81.230 | 113.081 | 174.936 | 1.00 | 52.47 |
| 2467 | O | VAL | B | 445 | 82.385 | 113.474 | 174.820 | 1.00 | 84.33 |
| 2468 | N | TYR | B | 446 | 80.917 | 111.877 | 175.406 | 1.00 | 58.93 |
| 2469 | CA | TYR | B | 446 | 81.928 | 110.913 | 175.817 | 1.00 | 94.16 |
| 2470 | CB | TYR | B | 446 | 82.051 | 110.897 | 177.344 | 1.00 | 114.12 |
| 2471 | CG | TYR | B | 446 | 82.132 | 112.276 | 177.967 | 1.00 | 123.21 |
| 2472 | CD1 | TYR | B | 446 | 81.042 | 112.826 | 178.628 | 1.00 | 107.03 |
| 2473 | CE1 | TYR | B | 446 | 81.091 | 114.111 | 179.161 | 1.00 | 140.23 |
| 2474 | CD2 | TYR | B | 446 | 83.288 | 113.048 | 177.854 | 1.00 | 175.76 |
| 2475 | CE2 | TYR | B | 446 | 83.347 | 114.338 | 178.384 | 1.00 | 166.71 |
| 2476 | CZ | TYR | B | 446 | 82.242 | 114.861 | 179.034 | 1.00 | 148.92 |
| 2477 | OH | TYR | B | 446 | 82.281 | 116.137 | 179.545 | 1.00 | 163.80 |
| 2478 | C | TYR | B | 446 | 81.515 | 109.532 | 175.325 | 1.00 | 98.27 |
| 2479 | O | TYR | B | 446 | 80.467 | 109.032 | 175.715 | 1.00 | 94.14 |
| 2480 | N | ALA | B | 447 | 82.337 | 108.917 | 174.476 | 1.00 | 102.60 |
| 2481 | CA | ALA | B | 447 | 82.035 | 107.592 | 173.925 | 1.00 | 60.72 |
| 2482 | CB | ALA | B | 447 | 82.452 | 107.548 | 172.479 | 1.00 | 85.56 |
| 2483 | C | ALA | B | 447 | 82.706 | 106.451 | 174.698 | 1.00 | 79.19 |
| 2484 | O | ALA | B | 447 | 83.836 | 106.585 | 175.166 | 1.00 | 86.10 |
| 2485 | N | PHE | B | 448 | 82.018 | 105.322 | 174.823 | 1.00 | 45.82 |
| 2486 | CA | PHE | B | 448 | 82.569 | 104.190 | 175.560 | 1.00 | 98.07 |
| 2487 | CB | PHE | B | 448 | 81.848 | 104.064 | 176.905 | 1.00 | 113.90 |
| 2488 | CG | PHE | B | 448 | 81.973 | 105.292 | 177.762 | 1.00 | 142.00 |
| 2489 | CD1 | PHE | B | 448 | 81.192 | 106.412 | 177.512 | 1.00 | 154.40 |
| 2490 | CD2 | PHE | B | 448 | 82.920 | 105.353 | 178.777 | 1.00 | 186.99 |
| 2491 | CE1 | PHE | B | 448 | 81.356 | 107.576 | 178.258 | 1.00 | 156.24 |
| 2492 | CE2 | PHE | B | 448 | 83.091 | 106.511 | 179.529 | 1.00 | 185.02 |
| 2493 | CZ | PHE | B | 448 | 82.309 | 107.625 | 179.268 | 1.00 | 183.09 |
| 2494 | C | PHE | B | 448 | 82.528 | 102.867 | 174.795 | 1.00 | 87.01 |
| 2495 | O | PHE | B | 448 | 82.229 | 102.854 | 173.608 | 1.00 | 104.18 |
| 2496 | N | ALA | B | 449 | 82.843 | 101.761 | 175.468 | 1.00 | 79.78 |
| 2497 | CA | ALA | B | 449 | 82.846 | 100.458 | 174.814 | 1.00 | 40.21 |
| 2498 | CB | ALA | B | 449 | 83.970 | 100.401 | 173.803 | 1.00 | 97.16 |
| 2499 | C | ALA | B | 449 | 82.959 | 99.284 | 175.780 | 1.00 | 54.65 |
| 2500 | O | ALA | B | 449 | 83.846 | 99.231 | 176.623 | 1.00 | 65.36 |
| 2501 | N | THR | B | 450 | 82.061 | 98.325 | 175.619 | 1.00 | 48.81 |
| 2502 | CA | THR | B | 450 | 81.993 | 97.144 | 176.467 | 1.00 | 85.23 |
| 2503 | CB | THR | B | 450 | 80.626 | 96.450 | 176.266 | 1.00 | 58.63 |
| 2504 | OG1 | THR | B | 450 | 79.578 | 97.340 | 176.657 | 1.00 | 98.59 |
| 2505 | CG2 | THR | B | 450 | 80.529 | 95.176 | 177.085 | 1.00 | 107.27 |
| 2506 | C | THR | B | 450 | 83.088 | 96.099 | 176.252 | 1.00 | 86.98 |
| 2507 | O | THR | B | 450 | 83.677 | 96.015 | 175.182 | 1.00 | 115.23 |
| 2508 | N | PRO | B | 451 | 83.398 | 95.317 | 177.298 | 1.00 | 82.22 |
| 2509 | CD | PRO | B | 451 | 83.248 | 95.823 | 178.669 | 1.00 | 78.05 |
| 2510 | CA | PRO | B | 451 | 84.401 | 94.247 | 177.274 | 1.00 | 102.30 |
| 2511 | CB | PRO | B | 451 | 84.895 | 94.197 | 178.715 | 1.00 | 127.73 |
| 2512 | CG | PRO | B | 451 | 84.606 | 95.567 | 179.233 | 1.00 | 117.16 |
| 2513 | C | PRO | B | 451 | 83.638 | 92.976 | 176.904 | 1.00 | 102.38 |
| 2514 | O | PRO | B | 451 | 82.434 | 92.893 | 177.140 | 1.00 | 107.16 |
| 2515 | N | GLU | B | 452 | 84.316 | 91.984 | 176.339 | 1.00 | 119.75 |
| 2516 | CA | GLU | B | 452 | 83.619 | 90.760 | 175.950 | 1.00 | 135.04 |
| 2517 | CB | GLU | B | 452 | 84.588 | 89.759 | 175.301 | 1.00 | 160.10 |
| 2518 | CG | GLU | B | 452 | 85.720 | 89.279 | 176.195 | 1.00 | 192.95 |
| 2519 | CD | GLU | B | 452 | 86.553 | 88.193 | 175.536 | 1.00 | 193.27 |
| 2520 | OE1 | GLU | B | 452 | 85.996 | 87.113 | 175.242 | 1.00 | 173.50 |
| 2521 | OE2 | GLU | B | 452 | 87.762 | 88.420 | 175.311 | 1.00 | 191.58 |
| 2522 | C | GLU | B | 452 | 82.901 | 90.097 | 177.120 | 1.00 | 120.59 |
| 2523 | O | GLU | B | 452 | 83.117 | 90.448 | 178.278 | 1.00 | 87.28 |
| 2524 | N | TRP | B | 453 | 82.040 | 89.138 | 176.797 | 1.00 | 120.79 |
| 2525 | CA | TRP | B | 453 | 81.274 | 88.403 | 177.793 | 1.00 | 141.89 |
| 2526 | CB | TRP | B | 453 | 79.909 | 89.074 | 177.986 | 1.00 | 165.76 |
| 2527 | CG | TRP | B | 453 | 78.970 | 88.353 | 178.913 | 1.00 | 194.36 |
| 2528 | CD2 | TRP | B | 453 | 78.780 | 88.606 | 180.312 | 1.00 | 210.98 |
| 2529 | CE2 | TRP | B | 453 | 77.810 | 87.688 | 180.773 | 1.00 | 213.28 |
| 2530 | CE3 | TRP | B | 453 | 79.338 | 89.515 | 181.221 | 1.00 | 215.87 |
| 2531 | CD1 | TRP | B | 453 | 78.130 | 87.324 | 178.595 | 1.00 | 204.35 |
| 2532 | NE1 | TRP | B | 453 | 77.429 | 86.920 | 179.705 | 1.00 | 205.41 |
| 2533 | CZ2 | TRP | B | 453 | 77.382 | 87.656 | 182.106 | 1.00 | 220.95 |
| 2534 | CZ3 | TRP | B | 453 | 78.913 | 89.483 | 182.547 | 1.00 | 216.68 |
| 2535 | CH2 | TRP | B | 453 | 77.945 | 88.557 | 182.975 | 1.00 | 222.83 |
| 2536 | C | TRP | B | 453 | 81.111 | 86.952 | 177.340 | 1.00 | 151.58 |
| 2537 | O | TRP | B | 453 | 80.894 | 86.679 | 176.161 | 1.00 | 148.39 |
| 2538 | N | PRO | B | 454 | 81.227 | 86.000 | 178.277 | 1.00 | 154.83 |
| 2539 | CD | PRO | B | 454 | 81.493 | 86.244 | 179.707 | 1.00 | 142.37 |
| 2540 | CA | PRO | B | 454 | 81.101 | 84.563 | 178.014 | 1.00 | 175.73 |
| 2541 | CB | PRO | B | 454 | 80.922 | 83.982 | 179.410 | 1.00 | 177.55 |
| 2542 | CG | PRO | B | 454 | 81.817 | 84.854 | 180.224 | 1.00 | 168.66 |
| 2543 | C | PRO | B | 454 | 79.962 | 84.171 | 177.075 | 1.00 | 189.74 |
| 2544 | O | PRO | B | 454 | 79.983 | 83.092 | 176.485 | 1.00 | 208.45 |
| 2545 | N | GLY | B | 455 | 78.971 | 85.045 | 176.945 | 1.00 | 196.85 |
| 2546 | CA | GLY | B | 455 | 77.842 | 84.759 | 176.078 | 1.00 | 187.96 |
| 2547 | C | GLY | B | 455 | 78.211 | 84.739 | 174.607 | 1.00 | 180.32 |
| 2548 | O | GLY | B | 455 | 77.762 | 83.871 | 173.859 | 1.00 | 192.44 |
| 2549 | N | SER | B | 456 | 79.029 | 85.701 | 174.192 | 1.00 | 169.42 |
| 2550 | CA | SER | B | 456 | 79.463 | 85.802 | 172.801 | 1.00 | 166.91 |
| 2551 | CB | SER | B | 456 | 78.471 | 86.631 | 171.992 | 1.00 | 158.61 |
| 2552 | OG | SER | B | 456 | 78.452 | 87.968 | 172.457 | 1.00 | 140.70 |
| 2553 | C | SER | B | 456 | 80.824 | 86.474 | 172.755 | 1.00 | 166.74 |
| 2554 | O | SER | B | 456 | 81.092 | 87.397 | 173.523 | 1.00 | 173.32 |
| 2555 | N | ARG | B | 457 | 81.677 | 86.022 | 171.844 | 1.00 | 174.22 |
| 2556 | CA | ARG | B | 457 | 83.016 | 86.582 | 171.723 | 1.00 | 176.54 |
| 2557 | CB | ARG | B | 457 | 84.057 | 85.452 | 171.727 | 1.00 | 182.95 |
| 2558 | CG | ARG | B | 457 | 83.970 | 84.538 | 172.946 | 1.00 | 199.86 |
| 2559 | CD | ARG | B | 457 | 85.036 | 83.449 | 172.934 | 1.00 | 203.45 |
| 2560 | NE | ARG | B | 457 | 84.881 | 82.538 | 174.067 | 1.00 | 215.80 |
| 2561 | CZ | ARG | B | 457 | 85.704 | 81.529 | 174.339 | 1.00 | 205.75 |
| 2562 | NH1 | ARG | B | 457 | 86.751 | 81.296 | 173.558 | 1.00 | 206.61 |
| 2563 | NH2 | ARG | B | 457 | 85.479 | 80.751 | 175.391 | 1.00 | 185.40 |
| 2564 | C | ARG | B | 457 | 83.180 | 87.431 | 170.468 | 1.00 | 160.43 |
| 2565 | O | ARG | B | 457 | 84.282 | 87.892 | 170.169 | 1.00 | 168.18 |
| 2566 | N | ASP | B | 458 | 82.090 | 87.647 | 169.737 | 1.00 | 145.98 |
| 2567 | CA | ASP | B | 458 | 82.161 | 88.438 | 168.512 | 1.00 | 164.53 |
| 2568 | CB | ASP | B | 458 | 82.048 | 87.527 | 167.289 | 1.00 | 187.11 |
| 2569 | CG | ASP | B | 458 | 83.221 | 86.575 | 167.162 | 1.00 | 204.04 |
| 2570 | OD1 | ASP | B | 458 | 84.377 | 87.051 | 167.162 | 1.00 | 205.31 |
| 2571 | OD2 | ASP | B | 458 | 82.989 | 85.352 | 167.062 | 1.00 | 200.56 |
| 2572 | C | ASP | B | 458 | 81.118 | 89.543 | 168.419 | 1.00 | 166.34 |
| 2573 | O | ASP | B | 458 | 80.827 | 90.043 | 167.329 | 1.00 | 100.86 |
| 2574 | N | LYS | B | 459 | 80.561 | 89.920 | 169.566 | 1.00 | 179.43 |
| 2575 | CA | LYS | B | 459 | 79.561 | 90.978 | 169.629 | 1.00 | 157.46 |
| 2576 | CB | LYS | B | 459 | 78.155 | 90.385 | 169.808 | 1.00 | 170.13 |
| 2577 | CG | LYS | B | 459 | 77.680 | 89.492 | 168.663 | 1.00 | 195.45 |
| 2578 | CD | LYS | B | 459 | 76.254 | 88.985 | 168.892 | 1.00 | 180.52 |
| 2579 | CE | LYS | B | 459 | 75.783 | 88.106 | 167.737 | 1.00 | 168.06 |
| 2580 | NZ | LYS | B | 459 | 74.380 | 87.632 | 167.911 | 1.00 | 140.21 |
| 2581 | C | LYS | B | 459 | 79.886 | 91.894 | 170.805 | 1.00 | 156.35 |
| 2582 | O | LYS | B | 459 | 79.922 | 91.450 | 171.953 | 1.00 | 165.29 |
| 2583 | N | ARG | B | 460 | 80.140 | 93.167 | 170.515 | 1.00 | 147.60 |
| 2584 | CA | ARG | B | 460 | 80.444 | 94.141 | 171.561 | 1.00 | 148.70 |
| 2585 | CB | ARG | B | 460 | 81.942 | 94.468 | 171.566 | 1.00 | 116.00 |
| 2586 | CG | ARG | B | 460 | 82.793 | 93.307 | 172.076 | 1.00 | 133.11 |
| 2587 | CD | ARG | B | 460 | 84.209 | 93.727 | 172.454 | 1.00 | 145.86 |
| 2588 | NE | ARG | B | 460 | 84.924 | 92.645 | 173.130 | 1.00 | 154.57 |
| 2589 | CZ | ARG | B | 460 | 86.106 | 92.776 | 173.728 | 1.00 | 176.49 |
| 2590 | NH1 | ARG | B | 460 | 86.723 | 93.949 | 173.741 | 1.00 | 156.00 |
| 2591 | NH2 | ARG | B | 460 | 86.673 | 91.733 | 174.320 | 1.00 | 195.46 |
| 2592 | C | ARG | B | 460 | 79.595 | 95.410 | 171.423 | 1.00 | 134.14 |
| 2593 | O | ARG | B | 460 | 79.384 | 95.915 | 170.320 | 1.00 | 108.53 |
| 2594 | N | THR | B | 461 | 79.124 | 95.914 | 172.562 | 1.00 | 97.76 |
| 2595 | CA | THR | B | 461 | 78.240 | 97.078 | 172.631 | 1.00 | 80.21 |
| 2596 | CB | THR | B | 461 | 77.224 | 96.860 | 173.750 | 1.00 | 88.28 |
| 2597 | OG1 | THR | B | 461 | 76.775 | 95.499 | 173.711 | 1.00 | 112.83 |
| 2598 | CG2 | THR | B | 461 | 76.037 | 97.804 | 173.597 | 1.00 | 61.89 |
| 2599 | C | THR | B | 461 | 78.845 | 98.467 | 172.849 | 1.00 | 76.50 |
| 2600 | O | THR | B | 461 | 79.691 | 98.647 | 173.719 | 1.00 | 62.71 |
| 2601 | N | LEU | B | 462 | 78.385 | 99.453 | 172.079 | 1.00 | 66.18 |
| 2602 | CA | LEU | B | 462 | 78.869 | 100.827 | 172.229 | 1.00 | 94.33 |
| 2603 | CB | LEU | B | 462 | 79.268 | 101.446 | 170.885 | 1.00 | 67.03 |
| 2604 | CG | LEU | B | 462 | 80.510 | 100.946 | 170.147 | 1.00 | 85.83 |
| 2605 | CD1 | LEU | B | 462 | 81.004 | 102.049 | 169.214 | 1.00 | 60.23 |
| 2606 | CD2 | LEU | B | 462 | 81.594 | 100.578 | 171.134 | 1.00 | 69.11 |
| 2607 | C | LEU | B | 462 | 77.811 | 101.721 | 172.867 | 1.00 | 93.33 |
| 2608 | O | LEU | B | 462 | 76.612 | 101.466 | 172.742 | 1.00 | 137.10 |
| 2609 | N | ALA | B | 463 | 78.267 | 102.777 | 173.538 | 1.00 | 84.75 |
| 2610 | CA | ALA | B | 463 | 77.385 | 103.730 | 174.207 | 1.00 | 57.78 |
| 2611 | CB | ALA | B | 463 | 77.231 | 103.369 | 175.655 | 1.00 | 67.02 |
| 2612 | C | ALA | B | 463 | 77.982 | 105.115 | 174.089 | 1.00 | 65.49 |
| 2613 | O | ALA | B | 463 | 79.197 | 105.283 | 174.138 | 1.00 | 79.68 |
| 2614 | N | CYS | B | 464 | 77.127 | 106.114 | 173.950 | 1.00 | 77.94 |
| 2615 | CA | CYS | B | 464 | 77.599 | 107.476 | 173.795 | 1.00 | 76.01 |
| 2616 | C | CYS | B | 464 | 76.707 | 108.413 | 174.573 | 1.00 | 97.04 |
| 2617 | O | CYS | B | 464 | 75.511 | 108.494 | 174.317 | 1.00 | 118.25 |
| 2618 | CB | CYS | B | 464 | 77.580 | 107.839 | 172.322 | 1.00 | 48.71 |
| 2619 | SG | CYS | B | 464 | 78.259 | 109.457 | 171.871 | 1.00 | 108.42 |
| 2620 | N | LEU | B | 465 | 77.300 | 109.120 | 175.526 | 1.00 | 104.12 |
| 2621 | CA | LEU | B | 465 | 76.568 | 110.055 | 176.368 | 1.00 | 83.50 |
| 2622 | CB | LEU | B | 465 | 76.945 | 109.819 | 177.827 | 1.00 | 57.15 |
| 2623 | CG | LEU | B | 465 | 76.704 | 110.946 | 178.820 | 1.00 | 72.22 |
| 2624 | CD1 | LEU | B | 465 | 75.332 | 111.558 | 178.626 | 1.00 | 80.58 |
| 2625 | CD2 | LEU | B | 465 | 76.872 | 110.378 | 180.214 | 1.00 | 78.64 |
| 2626 | C | LEU | B | 465 | 76.796 | 111.517 | 175.999 | 1.00 | 76.86 |
| 2627 | O | LEU | B | 465 | 77.918 | 111.999 | 175.967 | 1.00 | 62.37 |
| 2628 | N | ILE | B | 466 | 75.713 | 112.220 | 175.726 | 1.00 | 63.57 |
| 2629 | CA | ILE | B | 466 | 75.794 | 113.617 | 175.367 | 1.00 | 60.62 |
| 2630 | CB | ILE | B | 466 | 75.174 | 113.839 | 173.997 | 1.00 | 81.07 |
| 2631 | CG2 | ILE | B | 466 | 75.447 | 115.266 | 173.508 | 1.00 | 60.00 |
| 2632 | CG1 | ILE | B | 466 | 75.729 | 112.784 | 173.042 | 1.00 | 33.92 |
| 2633 | CD1 | ILE | B | 466 | 75.341 | 113.005 | 171.599 | 1.00 | 85.20 |
| 2634 | C | ILE | B | 466 | 75.029 | 114.398 | 176.420 | 1.00 | 93.44 |
| 2635 | O | ILE | B | 466 | 73.826 | 114.215 | 176.575 | 1.00 | 96.66 |
| 2636 | N | GLN | B | 467 | 75.723 | 115.271 | 177.142 | 1.00 | 102.53 |
| 2637 | CA | GLN | B | 467 | 75.072 | 116.033 | 178.195 | 1.00 | 81.78 |
| 2638 | CB | GLN | B | 467 | 75.418 | 115.422 | 179.552 | 1.00 | 45.68 |
| 2639 | CG | GLN | B | 467 | 76.895 | 115.224 | 179.783 | 1.00 | 79.30 |
| 2640 | CD | GLN | B | 467 | 77.217 | 114.845 | 181.222 | 1.00 | 113.12 |
| 2641 | OE1 | GLN | B | 467 | 76.587 | 113.958 | 181.809 | 1.00 | 73.47 |
| 2642 | NE2 | GLN | B | 467 | 78.213 | 115.514 | 181.794 | 1.00 | 129.90 |
| 2643 | C | GLN | B | 467 | 75.318 | 117.535 | 178.260 | 1.00 | 57.56 |
| 2644 | O | GLN | B | 467 | 75.964 | 118.136 | 177.400 | 1.00 | 58.32 |
| 2645 | N | ASN | B | 468 | 74.751 | 118.122 | 179.306 | 1.00 | 90.17 |
| 2646 | CA | ASN | B | 468 | 74.855 | 119.537 | 179.600 | 1.00 | 80.28 |
| 2647 | CB | ASN | B | 468 | 76.182 | 119.806 | 180.295 | 1.00 | 100.04 |
| 2648 | CG | ASN | B | 468 | 76.430 | 118.848 | 181.444 | 1.00 | 135.69 |
| 2649 | OD1 | ASN | B | 468 | 75.579 | 118.677 | 182.323 | 1.00 | 115.44 |
| 2650 | ND2 | ASN | B | 468 | 77.598 | 118.211 | 181.442 | 1.00 | 138.13 |
| 2651 | C | ASN | B | 468 | 74.695 | 120.436 | 178.395 | 1.00 | 92.36 |
| 2652 | O | ASN | B | 468 | 75.553 | 121.266 | 178.104 | 1.00 | 115.17 |
| 2653 | N | PHE | B | 469 | 73.582 | 120.273 | 177.696 | 1.00 | 53.18 |
| 2654 | CA | PHE | B | 469 | 73.314 | 121.103 | 176.537 | 1.00 | 71.01 |
| 2655 | CB | PHE | B | 469 | 73.459 | 120.295 | 175.246 | 1.00 | 56.66 |
| 2656 | CG | PHE | B | 469 | 72.531 | 119.107 | 175.155 | 1.00 | 100.03 |
| 2657 | CD1 | PHE | B | 469 | 71.424 | 119.130 | 174.306 | 1.00 | 45.65 |
| 2658 | CD2 | PHE | B | 469 | 72.781 | 117.952 | 175.895 | 1.00 | 97.64 |
| 2659 | CE1 | PHE | B | 469 | 70.593 | 118.026 | 174.192 | 1.00 | 97.74 |
| 2660 | CE2 | PHE | B | 469 | 71.946 | 116.838 | 175.785 | 1.00 | 35.93 |
| 2661 | CZ | PHE | B | 469 | 70.855 | 116.877 | 174.934 | 1.00 | 100.21 |
| 2662 | C | PHE | B | 469 | 71.917 | 121.672 | 176.618 | 1.00 | 72.62 |
| 2663 | O | PHE | B | 469 | 71.106 | 121.247 | 177.450 | 1.00 | 57.82 |
| 2664 | N | MET | B | 470 | 71.650 | 122.640 | 175.748 | 1.00 | 28.09 |
| 2665 | CA | MET | B | 470 | 70.344 | 123.287 | 175.681 | 1.00 | 59.88 |
| 2666 | CB | MET | B | 470 | 70.018 | 123.983 | 177.000 | 1.00 | 109.60 |
| 2667 | CG | MET | B | 470 | 71.139 | 124.822 | 177.586 | 1.00 | 125.93 |
| 2668 | SD | MET | B | 470 | 71.008 | 124.868 | 179.393 | 1.00 | 132.45 |
| 2669 | CE | MET | B | 470 | 69.604 | 125.930 | 179.600 | 1.00 | 165.70 |
| 2670 | C | MET | B | 470 | 70.355 | 124.265 | 174.524 | 1.00 | 62.44 |
| 2671 | O | MET | B | 470 | 71.359 | 124.924 | 174.279 | 1.00 | 61.44 |
| 2672 | N | PRO | B | 471 | 69.224 | 124.398 | 173.803 | 1.00 | 78.95 |
| 2673 | CD | PRO | B | 471 | 69.191 | 125.344 | 172.677 | 1.00 | 80.51 |
| 2674 | CA | PRO | B | 471 | 67.936 | 123.696 | 173.918 | 1.00 | 80.70 |
| 2675 | CB | PRO | B | 471 | 67.159 | 124.210 | 172.709 | 1.00 | 74.00 |
| 2676 | CG | PRO | B | 471 | 67.714 | 125.581 | 172.516 | 1.00 | 99.60 |
| 2677 | C | PRO | B | 471 | 68.028 | 122.180 | 173.923 | 1.00 | 80.76 |
| 2678 | O | PRO | B | 471 | 69.116 | 121.612 | 173.879 | 1.00 | 103.67 |
| 2679 | N | GLU | B | 472 | 66.879 | 121.518 | 173.962 | 1.00 | 88.76 |
| 2680 | CA | GLU | B | 472 | 66.873 | 120.070 | 173.975 | 1.00 | 85.51 |
| 2681 | CB | GLU | B | 472 | 65.665 | 119.551 | 174.757 | 1.00 | 112.19 |
| 2682 | CG | GLU | B | 472 | 64.326 | 120.100 | 174.287 | 1.00 | 171.67 |
| 2683 | CD | GLU | B | 472 | 63.145 | 119.321 | 174.848 | 1.00 | 191.16 |
| 2684 | OE1 | GLU | B | 472 | 63.088 | 119.123 | 176.083 | 1.00 | 177.52 |
| 2685 | OE2 | GLU | B | 472 | 62.272 | 118.908 | 174.053 | 1.00 | 195.05 |
| 2686 | C | GLU | B | 472 | 66.878 | 119.472 | 172.575 | 1.00 | 108.68 |
| 2687 | O | GLU | B | 472 | 67.002 | 118.258 | 172.429 | 1.00 | 108.87 |
| 2688 | N | ASP | B | 473 | 66.748 | 120.310 | 171.548 | 1.00 | 67.19 |
| 2689 | CA | ASP | B | 473 | 66.747 | 119.812 | 170.168 | 1.00 | 94.32 |
| 2690 | CB | ASP | B | 473 | 66.278 | 120.901 | 169.206 | 1.00 | 108.16 |
| 2691 | CG | ASP | B | 473 | 64.829 | 121.247 | 169.396 | 1.00 | 130.25 |
| 2692 | OD1 | ASP | B | 473 | 64.004 | 120.313 | 169.448 | 1.00 | 152.46 |
| 2693 | OD2 | ASP | B | 473 | 64.511 | 122.446 | 169.486 | 1.00 | 127.62 |
| 2694 | C | ASP | B | 473 | 68.118 | 119.302 | 169.725 | 1.00 | 86.57 |
| 2695 | O | ASP | B | 473 | 69.002 | 120.089 | 169.380 | 1.00 | 76.15 |
| 2696 | N | ILE | B | 474 | 68.283 | 117.982 | 169.704 | 1.00 | 86.59 |
| 2697 | CA | ILE | B | 474 | 69.559 | 117.388 | 169.332 | 1.00 | 62.99 |
| 2698 | CB | ILE | B | 474 | 70.315 | 116.903 | 170.597 | 1.00 | 70.94 |
| 2699 | CG2 | ILE | B | 474 | 69.789 | 115.557 | 171.052 | 1.00 | 49.54 |
| 2700 | CG1 | ILE | B | 474 | 71.798 | 116.740 | 170.301 | 1.00 | 79.49 |
| 2701 | CD1 | ILE | B | 474 | 72.587 | 116.274 | 171.507 | 1.00 | 78.48 |
| 2702 | C | ILE | B | 474 | 69.446 | 116.217 | 168.360 | 1.00 | 81.03 |
| 2703 | O | ILE | B | 474 | 68.513 | 115.412 | 168.436 | 1.00 | 79.23 |
| 2704 | N | SER | B | 475 | 70.410 | 116.134 | 167.448 | 1.00 | 80.57 |
| 2705 | CA | SER | B | 475 | 70.478 | 115.057 | 166.464 | 1.00 | 72.68 |
| 2706 | CB | SER | B | 475 | 70.451 | 115.624 | 165.044 | 1.00 | 48.43 |
| 2707 | OG | SER | B | 475 | 69.152 | 115.552 | 164.488 | 1.00 | 90.69 |
| 2708 | C | SER | B | 475 | 71.758 | 114.239 | 166.657 | 1.00 | 52.82 |
| 2709 | O | SER | B | 475 | 72.857 | 114.708 | 166.371 | 1.00 | 78.59 |
| 2710 | N | VAL | B | 476 | 71.611 | 113.020 | 167.154 | 1.00 | 56.94 |
| 2711 | CA | VAL | B | 476 | 72.746 | 112.127 | 167.364 | 1.00 | 46.67 |
| 2712 | CB | VAL | B | 476 | 72.522 | 111.246 | 168.605 | 1.00 | 59.60 |
| 2713 | CG1 | VAL | B | 476 | 73.590 | 110.197 | 168.732 | 1.00 | 49.38 |
| 2714 | CG2 | VAL | B | 476 | 72.520 | 112.094 | 169.816 | 1.00 | 76.03 |
| 2715 | C | VAL | B | 476 | 72.884 | 111.219 | 166.133 | 1.00 | 66.33 |
| 2716 | O | VAL | B | 476 | 71.938 | 111.062 | 165.354 | 1.00 | 113.57 |
| 2717 | N | GLN | B | 477 | 74.065 | 110.632 | 165.961 | 1.00 | 72.08 |
| 2718 | CA | GLN | B | 477 | 74.340 | 109.725 | 164.850 | 1.00 | 86.67 |
| 2719 | CB | GLN | B | 477 | 74.256 | 110.474 | 163.530 | 1.00 | 59.98 |
| 2720 | CG | GLN | B | 477 | 75.039 | 111.750 | 163.500 | 1.00 | 52.42 |
| 2721 | CD | GLN | B | 477 | 74.806 | 112.507 | 162.215 | 1.00 | 113.14 |
| 2722 | OE1 | GLN | B | 477 | 73.664 | 112.831 | 161.874 | 1.00 | 115.80 |
| 2723 | NE2 | GLN | B | 477 | 75.884 | 112.792 | 161.487 | 1.00 | 104.02 |
| 2724 | C | GLN | B | 477 | 75.712 | 109.077 | 164.987 | 1.00 | 71.96 |
| 2725 | O | GLN | B | 477 | 76.433 | 109.318 | 165.946 | 1.00 | 72.78 |
| 2726 | N | TRP | B | 478 | 76.071 | 108.231 | 164.038 | 1.00 | 74.54 |
| 2727 | CA | TRP | B | 478 | 77.371 | 107.594 | 164.114 | 1.00 | 91.24 |
| 2728 | CB | TRP | B | 478 | 77.254 | 106.181 | 164.656 | 1.00 | 30.97 |
| 2729 | CG | TRP | B | 478 | 76.687 | 106.064 | 166.023 | 1.00 | 64.60 |
| 2730 | CD2 | TRP | B | 478 | 77.409 | 105.759 | 167.220 | 1.00 | 55.53 |
| 2731 | CE2 | TRP | B | 478 | 76.461 | 105.528 | 168.231 | 1.00 | 51.61 |
| 2732 | CE3 | TRP | B | 478 | 78.767 | 105.646 | 167.531 | 1.00 | 88.58 |
| 2733 | CD1 | TRP | B | 478 | 75.371 | 106.032 | 166.355 | 1.00 | 68.44 |
| 2734 | NE1 | TRP | B | 478 | 75.222 | 105.702 | 167.676 | 1.00 | 76.50 |
| 2735 | CZ2 | TRP | B | 478 | 76.824 | 105.184 | 169.534 | 1.00 | 99.88 |
| 2736 | CZ3 | TRP | B | 478 | 79.129 | 105.302 | 168.828 | 1.00 | 73.67 |
| 2737 | CH2 | TRP | B | 478 | 78.159 | 105.074 | 169.812 | 1.00 | 68.61 |
| 2738 | C | TRP | B | 478 | 78.063 | 107.529 | 162.771 | 1.00 | 73.34 |
| 2739 | O | TRP | B | 478 | 77.417 | 107.546 | 161.726 | 1.00 | 112.30 |
| 2740 | N | LEU | B | 479 | 79.387 | 107.450 | 162.811 | 1.00 | 54.55 |
| 2741 | CA | LEU | B | 479 | 80.171 | 107.362 | 161.597 | 1.00 | 75.53 |
| 2742 | CB | LEU | B | 479 | 80.982 | 108.634 | 161.420 | 1.00 | 86.60 |
| 2743 | CG | LEU | B | 479 | 80.135 | 109.894 | 161.606 | 1.00 | 64.71 |
| 2744 | CD1 | LEU | B | 479 | 81.024 | 111.119 | 161.500 | 1.00 | 143.30 |
| 2745 | CD2 | LEU | B | 479 | 79.031 | 109.939 | 160.564 | 1.00 | 91.15 |
| 2746 | C | LEU | B | 479 | 81.081 | 106.160 | 161.743 | 1.00 | 90.65 |
| 2747 | O | LEU | B | 479 | 81.316 | 105.695 | 162.856 | 1.00 | 67.67 |
| 2748 | N | HIS | B | 480 | 81.582 | 105.652 | 160.621 | 1.00 | 130.81 |
| 2749 | CA | HIS | B | 480 | 82.460 | 104.487 | 160.635 | 1.00 | 114.42 |
| 2750 | CB | HIS | B | 480 | 81.646 | 103.225 | 160.911 | 1.00 | 109.89 |
| 2751 | CG | HIS | B | 480 | 82.423 | 101.954 | 160.772 | 1.00 | 101.08 |
| 2752 | CD2 | HIS | B | 480 | 82.120 | 100.790 | 160.152 | 1.00 | 114.03 |
| 2753 | ND1 | HIS | B | 480 | 83.644 | 101.759 | 161.379 | 1.00 | 82.41 |
| 2754 | CE1 | HIS | B | 480 | 84.057 | 100.527 | 161.144 | 1.00 | 108.15 |
| 2755 | NE2 | HIS | B | 480 | 83.151 | 99.917 | 160.402 | 1.00 | 119.50 |
| 2756 | C | HIS | B | 480 | 83.186 | 104.348 | 159.316 | 1.00 | 84.59 |
| 2757 | O | HIS | B | 480 | 82.657 | 103.811 | 158.351 | 1.00 | 86.39 |
| 2758 | N | ASN | B | 481 | 84.406 | 104.846 | 159.275 | 1.00 | 93.11 |
| 2759 | CA | ASN | B | 481 | 85.181 | 104.760 | 158.059 | 1.00 | 118.77 |
| 2760 | CB | ASN | B | 481 | 85.276 | 103.302 | 157.612 | 1.00 | 96.52 |
| 2761 | CG | ASN | B | 481 | 86.518 | 103.023 | 156.817 | 1.00 | 137.81 |
| 2762 | OD1 | ASN | B | 481 | 86.812 | 101.876 | 156.498 | 1.00 | 143.19 |
| 2763 | ND2 | ASN | B | 481 | 87.262 | 104.075 | 156.488 | 1.00 | 162.07 |
| 2764 | C | ASN | B | 481 | 84.527 | 105.608 | 156.973 | 1.00 | 70.50 |
| 2765 | O | ASN | B | 481 | 84.388 | 105.183 | 155.836 | 1.00 | 95.06 |
| 2766 | N | GLU | B | 482 | 84.126 | 106.814 | 157.345 | 1.00 | 75.74 |
| 2767 | CA | GLU | B | 482 | 83.512 | 107.746 | 156.415 | 1.00 | 123.11 |
| 2768 | CB | GLU | B | 482 | 84.424 | 107.940 | 155.198 | 1.00 | 121.91 |
| 2769 | CG | GLU | B | 482 | 85.873 | 108.306 | 155.542 | 1.00 | 142.86 |
| 2770 | CD | GLU | B | 482 | 86.012 | 109.651 | 156.248 | 1.00 | 171.49 |
| 2771 | OE1 | GLU | B | 482 | 85.641 | 110.686 | 155.650 | 1.00 | 170.24 |
| 2772 | OE2 | GLU | B | 482 | 86.497 | 109.672 | 157.401 | 1.00 | 170.99 |
| 2773 | C | GLU | B | 482 | 82.130 | 107.277 | 155.969 | 1.00 | 104.98 |
| 2774 | O | GLU | B | 482 | 81.647 | 107.661 | 154.902 | 1.00 | 66.15 |
| 2775 | N | VAL | B | 483 | 81.490 | 106.457 | 156.795 | 1.00 | 66.23 |
| 2776 | CA | VAL | B | 483 | 80.164 | 105.948 | 156.472 | 1.00 | 92.18 |
| 2777 | CB | VAL | B | 483 | 80.210 | 104.427 | 156.245 | 1.00 | 99.60 |
| 2778 | CG1 | VAL | B | 483 | 78.813 | 103.869 | 156.069 | 1.00 | 96.51 |
| 2779 | CG2 | VAL | B | 483 | 81.050 | 104.125 | 155.022 | 1.00 | 173.41 |
| 2780 | C | VAL | B | 483 | 79.140 | 106.250 | 157.562 | 1.00 | 113.67 |
| 2781 | O | VAL | B | 483 | 79.184 | 105.661 | 158.640 | 1.00 | 139.78 |
| 2782 | N | GLN | B | 484 | 78.214 | 107.164 | 157.276 | 1.00 | 105.77 |
| 2783 | CA | GLN | B | 484 | 77.165 | 107.533 | 158.231 | 1.00 | 69.76 |
| 2784 | CB | GLN | B | 484 | 76.416 | 108.768 | 157.735 | 1.00 | 107.20 |
| 2785 | CG | GLN | B | 484 | 75.352 | 109.285 | 158.681 | 1.00 | 78.17 |
| 2786 | CD | GLN | B | 484 | 74.564 | 110.423 | 158.077 | 1.00 | 122.38 |
| 2787 | OE1 | GLN | B | 484 | 75.132 | 111.338 | 157.474 | 1.00 | 88.01 |
| 2788 | NE2 | GLN | B | 484 | 73.248 | 110.378 | 158.238 | 1.00 | 132.45 |
| 2789 | C | GLN | B | 484 | 76.177 | 106.385 | 158.392 | 1.00 | 52.68 |
| 2790 | O | GLN | B | 484 | 75.234 | 106.268 | 157.616 | 1.00 | 96.63 |
| 2791 | N | LEU | B | 485 | 76.386 | 105.553 | 159.408 | 1.00 | 54.65 |
| 2792 | CA | LEU | B | 485 | 75.529 | 104.401 | 159.636 | 1.00 | 61.68 |
| 2793 | CB | LEU | B | 485 | 75.859 | 103.754 | 160.976 | 1.00 | 56.33 |
| 2794 | CG | LEU | B | 485 | 77.220 | 103.068 | 161.101 | 1.00 | 54.48 |
| 2795 | CD1 | LEU | B | 485 | 77.171 | 102.084 | 162.246 | 1.00 | 62.35 |
| 2796 | CD2 | LEU | B | 485 | 77.554 | 102.325 | 159.813 | 1.00 | 115.96 |
| 2797 | C | LEU | B | 485 | 74.025 | 104.629 | 159.534 | 1.00 | 77.38 |
| 2798 | O | LEU | B | 485 | 73.537 | 105.746 | 159.679 | 1.00 | 81.23 |
| 2799 | N | PRO | B | 486 | 73.271 | 103.551 | 159.266 | 1.00 | 73.49 |
| 2800 | CD | PRO | B | 486 | 73.794 | 102.217 | 158.934 | 1.00 | 122.28 |
| 2801 | CA | PRO | B | 486 | 71.819 | 103.570 | 159.127 | 1.00 | 82.90 |
| 2802 | CB | PRO | B | 486 | 71.492 | 102.137 | 158.712 | 1.00 | 119.65 |
| 2803 | CG | PRO | B | 486 | 72.729 | 101.692 | 158.018 | 1.00 | 136.98 |
| 2804 | C | PRO | B | 486 | 71.154 | 103.952 | 160.433 | 1.00 | 94.37 |
| 2805 | O | PRO | B | 486 | 71.429 | 103.370 | 161.481 | 1.00 | 68.92 |
| 2806 | N | ASP | B | 487 | 70.268 | 104.932 | 160.359 | 1.00 | 84.31 |
| 2807 | CA | ASP | B | 487 | 69.560 | 105.398 | 161.533 | 1.00 | 88.88 |
| 2808 | CB | ASP | B | 487 | 68.424 | 106.329 | 161.097 | 1.00 | 108.41 |
| 2809 | CG | ASP | B | 487 | 68.070 | 107.347 | 162.155 | 1.00 | 154.62 |
| 2810 | OD1 | ASP | B | 487 | 67.549 | 106.946 | 163.217 | 1.00 | 160.64 |
| 2811 | OD2 | ASP | B | 487 | 68.322 | 108.549 | 161.926 | 1.00 | 168.53 |
| 2812 | C | ASP | B | 487 | 69.014 | 104.240 | 162.374 | 1.00 | 63.64 |
| 2813 | O | ASP | B | 487 | 69.085 | 104.269 | 163.596 | 1.00 | 90.99 |
| 2814 | N | ALA | B | 488 | 68.492 | 103.210 | 161.721 | 1.00 | 64.28 |
| 2815 | CA | ALA | B | 488 | 67.910 | 102.073 | 162.432 | 1.00 | 73.64 |
| 2816 | CB | ALA | B | 488 | 67.073 | 101.232 | 161.463 | 1.00 | 116.29 |
| 2817 | C | ALA | B | 488 | 68.921 | 101.189 | 163.143 | 1.00 | 75.09 |
| 2818 | O | ALA | B | 488 | 68.563 | 100.164 | 163.722 | 1.00 | 61.17 |
| 2819 | N | ARG | B | 489 | 70.180 | 101.599 | 163.122 | 1.00 | 47.46 |
| 2820 | CA | ARG | B | 489 | 71.245 | 100.817 | 163.739 | 1.00 | 76.59 |
| 2821 | CB | ARG | B | 489 | 72.562 | 101.088 | 163.014 | 1.00 | 92.18 |
| 2822 | CG | ARG | B | 489 | 72.771 | 100.227 | 161.785 | 1.00 | 116.98 |
| 2823 | CD | ARG | B | 489 | 73.469 | 98.935 | 162.162 | 1.00 | 101.72 |
| 2824 | NE | ARG | B | 489 | 74.904 | 99.013 | 161.904 | 1.00 | 96.78 |
| 2825 | CZ | ARG | B | 489 | 75.803 | 98.212 | 162.459 | 1.00 | 108.26 |
| 2826 | NH1 | ARG | B | 489 | 75.418 | 97.273 | 163.311 | 1.00 | 104.09 |
| 2827 | NH2 | ARG | B | 489 | 77.084 | 98.345 | 162.157 | 1.00 | 106.78 |
| 2828 | C | ARG | B | 489 | 71.432 | 101.066 | 165.219 | 1.00 | 78.64 |
| 2829 | O | ARG | B | 489 | 71.773 | 100.153 | 165.972 | 1.00 | 80.15 |
| 2830 | N | HIS | B | 490 | 71.205 | 102.308 | 165.631 | 1.00 | 85.57 |
| 2831 | CA | HIS | B | 490 | 71.377 | 102.703 | 167.023 | 1.00 | 79.08 |
| 2832 | CB | HIS | B | 490 | 72.359 | 103.863 | 167.106 | 1.00 | 65.14 |
| 2833 | CG | HIS | B | 490 | 71.883 | 105.094 | 166.405 | 1.00 | 51.35 |
| 2834 | CD2 | HIS | B | 490 | 70.847 | 105.924 | 166.669 | 1.00 | 81.18 |
| 2835 | ND1 | HIS | B | 490 | 72.493 | 105.592 | 165.276 | 1.00 | 77.41 |
| 2836 | CE1 | HIS | B | 490 | 71.855 | 106.678 | 164.874 | 1.00 | 76.90 |
| 2837 | NE2 | HIS | B | 490 | 70.852 | 106.901 | 165.703 | 1.00 | 118.70 |
| 2838 | C | HIS | B | 490 | 70.086 | 103.134 | 167.690 | 1.00 | 75.63 |
| 2839 | O | HIS | B | 490 | 69.136 | 103.545 | 167.028 | 1.00 | 82.18 |
| 2840 | N | SER | B | 491 | 70.072 | 103.056 | 169.017 | 1.00 | 111.19 |
| 2841 | CA | SER | B | 491 | 68.915 | 103.461 | 169.801 | 1.00 | 98.54 |
| 2842 | CB | SER | B | 491 | 68.477 | 102.333 | 170.737 | 1.00 | 70.59 |
| 2843 | OG | SER | B | 491 | 67.305 | 102.703 | 171.444 | 1.00 | 151.07 |
| 2844 | C | SER | B | 491 | 69.295 | 104.683 | 170.624 | 1.00 | 91.58 |
| 2845 | O | SER | B | 491 | 70.209 | 104.630 | 171.439 | 1.00 | 69.75 |
| 2846 | N | THR | B | 492 | 68.600 | 105.790 | 170.402 | 1.00 | 103.48 |
| 2847 | CA | THR | B | 492 | 68.874 | 107.011 | 171.149 | 1.00 | 84.76 |
| 2848 | CB | THR | B | 492 | 69.182 | 108.171 | 170.208 | 1.00 | 96.92 |
| 2849 | OG1 | THR | B | 492 | 70.431 | 107.927 | 169.553 | 1.00 | 98.55 |
| 2850 | CG2 | THR | B | 492 | 69.260 | 109.473 | 170.978 | 1.00 | 89.03 |
| 2851 | C | THR | B | 492 | 67.666 | 107.370 | 172.002 | 1.00 | 95.54 |
| 2852 | O | THR | B | 492 | 66.532 | 107.352 | 171.523 | 1.00 | 110.69 |
| 2853 | N | THR | B | 493 | 67.907 | 107.702 | 173.266 | 1.00 | 65.05 |
| 2854 | CA | THR | B | 493 | 66.809 | 108.036 | 174.159 | 1.00 | 70.59 |
| 2855 | CB | THR | B | 493 | 67.188 | 107.882 | 175.635 | 1.00 | 74.90 |
| 2856 | OG1 | THR | B | 493 | 68.008 | 108.984 | 176.019 | 1.00 | 55.82 |
| 2857 | CG2 | THR | B | 493 | 67.929 | 106.576 | 175.874 | 1.00 | 80.05 |
| 2858 | C | THR | B | 493 | 66.315 | 109.457 | 173.978 | 1.00 | 81.61 |
| 2859 | O | THR | B | 493 | 66.765 | 110.190 | 173.102 | 1.00 | 64.79 |
| 2860 | N | GLN | B | 494 | 65.365 | 109.834 | 174.820 | 1.00 | 97.42 |
| 2861 | CA | GLN | B | 494 | 64.806 | 111.166 | 174.769 | 1.00 | 90.31 |
| 2862 | CB | GLN | B | 494 | 63.296 | 111.115 | 175.033 | 1.00 | 108.14 |
| 2863 | CG | GLN | B | 494 | 62.486 | 110.334 | 174.005 | 1.00 | 116.36 |
| 2864 | CD | GLN | B | 494 | 62.660 | 110.860 | 172.584 | 1.00 | 154.38 |
| 2865 | OE1 | GLN | B | 494 | 62.652 | 112.070 | 172.350 | 1.00 | 129.72 |
| 2866 | NE2 | GLN | B | 494 | 62.804 | 109.947 | 171.627 | 1.00 | 166.89 |
| 2867 | C | GLN | B | 494 | 65.498 | 112.012 | 175.833 | 1.00 | 107.40 |
| 2868 | O | GLN | B | 494 | 65.869 | 111.509 | 176.900 | 1.00 | 110.85 |
| 2869 | N | PRO | B | 495 | 65.697 | 113.309 | 175.547 | 1.00 | 81.82 |
| 2870 | CD | PRO | B | 495 | 65.416 | 113.972 | 174.261 | 1.00 | 69.03 |
| 2871 | CA | PRO | B | 495 | 66.342 | 114.240 | 176.470 | 1.00 | 65.90 |
| 2872 | CB | PRO | B | 495 | 66.109 | 115.583 | 175.802 | 1.00 | 63.09 |
| 2873 | CG | PRO | B | 495 | 66.235 | 115.239 | 174.363 | 1.00 | 52.88 |
| 2874 | C | PRO | B | 495 | 65.742 | 114.186 | 177.867 | 1.00 | 75.89 |
| 2875 | O | PRO | B | 495 | 64.558 | 113.914 | 178.039 | 1.00 | 107.23 |
| 2876 | N | ARG | B | 496 | 66.580 | 114.437 | 178.862 | 1.00 | 112.34 |
| 2877 | CA | ARG | B | 496 | 66.166 | 114.445 | 180.257 | 1.00 | 123.31 |
| 2878 | CB | ARG | B | 496 | 66.434 | 113.080 | 180.902 | 1.00 | 145.69 |
| 2879 | CG | ARG | B | 496 | 65.366 | 112.031 | 180.599 | 1.00 | 159.05 |
| 2880 | CD | ARG | B | 496 | 65.774 | 110.643 | 181.091 | 1.00 | 178.53 |
| 2881 | NE | ARG | B | 496 | 64.643 | 109.715 | 181.137 | 1.00 | 209.19 |
| 2882 | CZ | ARG | B | 496 | 63.715 | 109.707 | 182.092 | 1.00 | 207.25 |
| 2883 | NH1 | ARG | B | 496 | 63.779 | 110.578 | 183.092 | 1.00 | 201.73 |
| 2884 | NH2 | ARG | B | 496 | 62.717 | 108.832 | 182.044 | 1.00 | 194.79 |
| 2885 | C | ARG | B | 496 | 66.963 | 115.549 | 180.944 | 1.00 | 134.53 |
| 2886 | O | ARG | B | 496 | 68.121 | 115.798 | 180.597 | 1.00 | 112.03 |
| 2887 | N | LYS | B | 497 | 66.341 | 116.220 | 181.907 | 1.00 | 136.67 |
| 2888 | CA | LYS | B | 497 | 66.999 | 117.318 | 182.601 | 1.00 | 99.35 |
| 2889 | CB | LYS | B | 497 | 65.955 | 118.224 | 183.252 | 1.00 | 115.41 |
| 2890 | CG | LYS | B | 497 | 64.939 | 118.793 | 182.278 | 1.00 | 150.70 |
| 2891 | CD | LYS | B | 497 | 63.952 | 119.718 | 182.976 | 1.00 | 164.39 |
| 2892 | CE | LYS | B | 497 | 62.907 | 120.251 | 182.003 | 1.00 | 162.23 |
| 2893 | NZ | LYS | B | 497 | 61.957 | 121.204 | 182.648 | 1.00 | 146.92 |
| 2894 | C | LYS | B | 497 | 68.005 | 116.876 | 183.649 | 1.00 | 102.60 |
| 2895 | O | LYS | B | 497 | 67.966 | 115.743 | 184.129 | 1.00 | 95.44 |
| 2896 | N | THR | B | 498 | 68.906 | 117.792 | 183.993 | 1.00 | 121.19 |
| 2897 | CA | THR | B | 498 | 69.940 | 117.543 | 184.989 | 1.00 | 137.86 |
| 2898 | CB | THR | B | 498 | 71.330 | 117.388 | 184.326 | 1.00 | 127.01 |
| 2899 | OG1 | THR | B | 498 | 71.571 | 118.479 | 183.426 | 1.00 | 102.85 |
| 2900 | CG2 | THR | B | 498 | 71.398 | 116.094 | 183.555 | 1.00 | 140.90 |
| 2901 | C | THR | B | 498 | 69.995 | 118.681 | 186.008 | 1.00 | 162.55 |
| 2902 | O | THR | B | 498 | 69.507 | 119.788 | 185.754 | 1.00 | 135.49 |
| 2903 | N | LYS | B | 499 | 70.589 | 118.405 | 187.164 | 1.00 | 151.73 |
| 2904 | CA | LYS | B | 499 | 70.701 | 119.410 | 188.212 | 1.00 | 168.61 |
| 2905 | CB | LYS | B | 499 | 71.167 | 118.755 | 189.520 | 1.00 | 183.90 |
| 2906 | CG | LYS | B | 499 | 70.993 | 119.632 | 190.759 | 1.00 | 193.98 |
| 2907 | CD | LYS | B | 499 | 71.224 | 118.849 | 192.051 | 1.00 | 178.03 |
| 2908 | CE | LYS | B | 499 | 70.926 | 119.704 | 193.283 | 1.00 | 168.01 |
| 2909 | NZ | LYS | B | 499 | 71.031 | 118.937 | 194.562 | 1.00 | 137.47 |
| 2910 | C | LYS | B | 499 | 71.677 | 120.509 | 187.780 | 1.00 | 173.90 |
| 2911 | O | LYS | B | 499 | 72.200 | 121.257 | 188.606 | 1.00 | 189.46 |
| 2912 | N | GLY | B | 500 | 71.910 | 120.597 | 186.473 | 1.00 | 172.18 |
| 2913 | CA | GLY | B | 500 | 72.811 | 121.601 | 185.936 | 1.00 | 169.02 |
| 2914 | C | GLY | B | 500 | 72.141 | 122.406 | 184.837 | 1.00 | 176.17 |
| 2915 | O | GLY | B | 500 | 72.813 | 122.959 | 183.965 | 1.00 | 182.74 |
| 2916 | N | SER | B | 501 | 70.810 | 122.456 | 184.884 | 1.00 | 151.02 |
| 2917 | CA | SER | B | 501 | 69.989 | 123.189 | 183.919 | 1.00 | 147.04 |
| 2918 | CB | SER | B | 501 | 70.242 | 124.698 | 184.044 | 1.00 | 152.23 |
| 2919 | OG | SER | B | 501 | 71.556 | 125.048 | 183.642 | 1.00 | 182.63 |
| 2920 | C | SER | B | 501 | 70.164 | 122.762 | 182.458 | 1.00 | 151.49 |
| 2921 | O | SER | B | 501 | 69.620 | 123.395 | 181.549 | 1.00 | 133.52 |
| 2922 | N | GLY | B | 502 | 70.916 | 121.691 | 182.230 | 1.00 | 142.82 |
| 2923 | CA | GLY | B | 502 | 71.116 | 121.218 | 180.873 | 1.00 | 95.52 |
| 2924 | C | GLY | B | 502 | 70.507 | 119.842 | 180.705 | 1.00 | 118.00 |
| 2925 | O | GLY | B | 502 | 70.114 | 119.219 | 181.689 | 1.00 | 106.89 |
| 2926 | N | PHE | B | 503 | 70.421 | 119.358 | 179.469 | 1.00 | 108.44 |
| 2927 | CA | PHE | B | 503 | 69.850 | 118.035 | 179.231 | 1.00 | 93.09 |
| 2928 | CB | PHE | B | 503 | 68.868 | 118.051 | 178.071 | 1.00 | 70.27 |
| 2929 | CG | PHE | B | 503 | 67.813 | 119.092 | 178.173 | 1.00 | 78.42 |
| 2930 | CD1 | PHE | B | 503 | 68.002 | 120.346 | 177.604 | 1.00 | 94.67 |
| 2931 | CD2 | PHE | B | 503 | 66.605 | 118.803 | 178.782 | 1.00 | 58.13 |
| 2932 | CE1 | PHE | B | 503 | 66.990 | 121.300 | 177.635 | 1.00 | 98.84 |
| 2933 | CE2 | PHE | B | 503 | 65.587 | 119.749 | 178.819 | 1.00 | 113.56 |
| 2934 | CZ | PHE | B | 503 | 65.779 | 121.001 | 178.243 | 1.00 | 112.26 |
| 2935 | C | PHE | B | 503 | 70.909 | 116.995 | 178.911 | 1.00 | 91.24 |
| 2936 | O | PHE | B | 503 | 72.074 | 117.326 | 178.682 | 1.00 | 63.55 |
| 2937 | N | PHE | B | 504 | 70.474 | 115.737 | 178.867 | 1.00 | 71.28 |
| 2938 | CA | PHE | B | 504 | 71.354 | 114.618 | 178.578 | 1.00 | 66.45 |
| 2939 | CB | PHE | B | 504 | 71.965 | 114.102 | 179.879 | 1.00 | 66.76 |
| 2940 | CG | PHE | B | 504 | 71.102 | 113.121 | 180.617 | 1.00 | 55.05 |
| 2941 | CD1 | PHE | B | 504 | 71.121 | 111.776 | 180.286 | 1.00 | 75.50 |
| 2942 | CD2 | PHE | B | 504 | 70.308 | 113.532 | 181.672 | 1.00 | 92.55 |
| 2943 | CE1 | PHE | B | 504 | 70.367 | 110.853 | 181.003 | 1.00 | 87.60 |
| 2944 | CE2 | PHE | B | 504 | 69.551 | 112.614 | 182.394 | 1.00 | 119.03 |
| 2945 | CZ | PHE | B | 504 | 69.583 | 111.271 | 182.057 | 1.00 | 113.46 |
| 2946 | C | PHE | B | 504 | 70.627 | 113.477 | 177.864 | 1.00 | 71.40 |
| 2947 | O | PHE | B | 504 | 69.534 | 113.078 | 178.262 | 1.00 | 98.42 |
| 2948 | N | VAL | B | 505 | 71.240 | 112.949 | 176.809 | 1.00 | 93.14 |
| 2949 | CA | VAL | B | 505 | 70.652 | 111.844 | 176.061 | 1.00 | 85.70 |
| 2950 | CB | VAL | B | 505 | 70.169 | 112.299 | 174.672 | 1.00 | 64.09 |
| 2951 | CG1 | VAL | B | 505 | 71.345 | 112.676 | 173.807 | 1.00 | 32.54 |
| 2952 | CG2 | VAL | B | 505 | 69.357 | 111.195 | 174.029 | 1.00 | 103.96 |
| 2953 | C | VAL | B | 505 | 71.669 | 110.722 | 175.888 | 1.00 | 58.13 |
| 2954 | O | VAL | B | 505 | 72.859 | 110.973 | 175.798 | 1.00 | 86.55 |
| 2955 | N | PHE | B | 506 | 71.187 | 109.487 | 175.841 | 1.00 | 81.98 |
| 2956 | CA | PHE | B | 506 | 72.035 | 108.311 | 175.685 | 1.00 | 63.87 |
| 2957 | CB | PHE | B | 506 | 71.733 | 107.331 | 176.792 | 1.00 | 50.64 |
| 2958 | CG | PHE | B | 506 | 72.585 | 107.497 | 178.006 | 1.00 | 61.69 |
| 2959 | CD1 | PHE | B | 506 | 72.100 | 107.129 | 179.255 | 1.00 | 87.69 |
| 2960 | CD2 | PHE | B | 506 | 73.900 | 107.921 | 177.902 | 1.00 | 101.17 |
| 2961 | CE1 | PHE | B | 506 | 72.904 | 107.168 | 180.385 | 1.00 | 55.65 |
| 2962 | CE2 | PHE | B | 506 | 74.721 | 107.965 | 179.032 | 1.00 | 102.15 |
| 2963 | CZ | PHE | B | 506 | 74.218 | 107.584 | 180.275 | 1.00 | 83.88 |
| 2964 | C | PHE | B | 506 | 71.816 | 107.605 | 174.357 | 1.00 | 71.24 |
| 2965 | O | PHE | B | 506 | 70.699 | 107.567 | 173.845 | 1.00 | 133.97 |
| 2966 | N | SER | B | 507 | 72.883 | 107.036 | 173.803 | 1.00 | 84.07 |
| 2967 | CA | SER | B | 507 | 72.801 | 106.312 | 172.533 | 1.00 | 76.16 |
| 2968 | CB | SER | B | 507 | 73.454 | 107.090 | 171.405 | 1.00 | 27.10 |
| 2969 | OG | SER | B | 507 | 73.355 | 106.340 | 170.220 | 1.00 | 58.51 |
| 2970 | C | SER | B | 507 | 73.480 | 104.962 | 172.635 | 1.00 | 57.27 |
| 2971 | O | SER | B | 507 | 74.493 | 104.821 | 173.310 | 1.00 | 52.02 |
| 2972 | N | ARG | B | 508 | 72.924 | 103.967 | 171.958 | 1.00 | 51.27 |
| 2973 | CA | ARG | B | 508 | 73.476 | 102.616 | 172.002 | 1.00 | 53.55 |
| 2974 | CB | ARG | B | 508 | 72.623 | 101.735 | 172.911 | 1.00 | 44.14 |
| 2975 | CG | ARG | B | 508 | 72.985 | 100.277 | 172.923 | 1.00 | 46.22 |
| 2976 | CD | ARG | B | 508 | 72.144 | 99.540 | 173.949 | 1.00 | 70.79 |
| 2977 | NE | ARG | B | 508 | 72.340 | 98.093 | 173.925 | 1.00 | 49.24 |
| 2978 | CZ | ARG | B | 508 | 71.855 | 97.291 | 172.984 | 1.00 | 103.44 |
| 2979 | NH1 | ARG | B | 508 | 71.147 | 97.796 | 171.984 | 1.00 | 133.71 |
| 2980 | NH2 | ARG | B | 508 | 72.064 | 95.983 | 173.054 | 1.00 | 124.77 |
| 2981 | C | ARG | B | 508 | 73.541 | 102.032 | 170.601 | 1.00 | 62.86 |
| 2982 | O | ARG | B | 508 | 72.555 | 102.020 | 169.864 | 1.00 | 86.63 |
| 2983 | N | LEU | B | 509 | 74.718 | 101.531 | 170.248 | 1.00 | 101.57 |
| 2984 | CA | LEU | B | 509 | 74.962 | 100.979 | 168.925 | 1.00 | 91.96 |
| 2985 | CB | LEU | B | 509 | 75.820 | 101.966 | 168.140 | 1.00 | 57.86 |
| 2986 | CG | LEU | B | 509 | 76.358 | 101.480 | 166.804 | 1.00 | 69.57 |
| 2987 | CD1 | LEU | B | 509 | 75.215 | 100.940 | 165.972 | 1.00 | 115.82 |
| 2988 | CD2 | LEU | B | 509 | 77.045 | 102.618 | 166.093 | 1.00 | 47.22 |
| 2989 | C | LEU | B | 509 | 75.656 | 99.623 | 168.930 | 1.00 | 72.22 |
| 2990 | O | LEU | B | 509 | 76.871 | 99.577 | 168.926 | 1.00 | 65.45 |
| 2991 | N | GLU | B | 510 | 74.902 | 98.526 | 168.928 | 1.00 | 103.54 |
| 2992 | CA | GLU | B | 510 | 75.513 | 97.191 | 168.921 | 1.00 | 79.97 |
| 2993 | CB | GLU | B | 510 | 74.427 | 96.107 | 168.847 | 1.00 | 105.98 |
| 2994 | CG | GLU | B | 510 | 73.491 | 96.056 | 170.061 | 1.00 | 124.66 |
| 2995 | CD | GLU | B | 510 | 72.349 | 95.050 | 169.905 | 1.00 | 156.13 |
| 2996 | OE1 | GLU | B | 510 | 71.488 | 95.247 | 169.018 | 1.00 | 161.88 |
| 2997 | OE2 | GLU | B | 510 | 72.309 | 94.061 | 170.673 | 1.00 | 131.37 |
| 2998 | C | GLU | B | 510 | 76.445 | 97.095 | 167.708 | 1.00 | 100.86 |
| 2999 | O | GLU | B | 510 | 76.171 | 97.705 | 166.671 | 1.00 | 97.53 |
| 3000 | N | VAL | B | 511 | 77.540 | 96.339 | 167.834 | 1.00 | 86.41 |
| 3001 | CA | VAL | B | 511 | 78.512 | 96.196 | 166.741 | 1.00 | 113.22 |
| 3002 | CB | VAL | B | 511 | 79.763 | 97.091 | 166.993 | 1.00 | 29.35 |
| 3003 | CG1 | VAL | B | 511 | 80.823 | 96.827 | 165.953 | 1.00 | 119.74 |
| 3004 | CG2 | VAL | B | 511 | 79.373 | 98.544 | 166.915 | 1.00 | 108.86 |
| 3005 | C | VAL | B | 511 | 78.981 | 94.760 | 166.461 | 1.00 | 152.83 |
| 3006 | O | VAL | B | 511 | 78.941 | 93.899 | 167.343 | 1.00 | 151.08 |
| 3007 | N | THR | B | 512 | 79.428 | 94.522 | 165.225 | 1.00 | 167.98 |
| 3008 | CA | THR | B | 512 | 79.908 | 93.212 | 164.786 | 1.00 | 153.93 |
| 3009 | CB | THR | B | 512 | 79.252 | 92.806 | 163.446 | 1.00 | 164.28 |
| 3010 | OG1 | THR | B | 512 | 77.826 | 92.831 | 163.583 | 1.00 | 167.99 |
| 3011 | CG2 | THR | B | 512 | 79.691 | 91.403 | 163.034 | 1.00 | 183.03 |
| 3012 | C | THR | B | 512 | 81.427 | 93.180 | 164.604 | 1.00 | 134.07 |
| 3013 | O | THR | B | 512 | 82.018 | 94.132 | 164.093 | 1.00 | 85.13 |
| 3014 | N | ARG | B | 513 | 82.042 | 92.071 | 165.017 | 1.00 | 128.25 |
| 3015 | CA | ARG | B | 513 | 83.488 | 91.879 | 164.910 | 1.00 | 132.09 |
| 3016 | CB | ARG | B | 513 | 83.853 | 90.401 | 165.116 | 1.00 | 158.22 |
| 3017 | CG | ARG | B | 513 | 85.357 | 90.120 | 165.061 | 1.00 | 185.92 |
| 3018 | CD | ARG | B | 513 | 85.684 | 88.632 | 165.174 | 1.00 | 210.68 |
| 3019 | NE | ARG | B | 513 | 87.125 | 88.379 | 165.106 | 1.00 | 235.05 |
| 3020 | CZ | ARG | B | 513 | 87.683 | 87.170 | 165.129 | 1.00 | 237.14 |
| 3021 | NH1 | ARG | B | 513 | 86.926 | 86.085 | 165.219 | 1.00 | 231.50 |
| 3022 | NH2 | ARG | B | 513 | 89.003 | 87.045 | 165.060 | 1.00 | 226.13 |
| 3023 | C | ARG | B | 513 | 84.028 | 92.345 | 163.566 | 1.00 | 119.35 |
| 3024 | O | ARG | B | 513 | 84.945 | 93.157 | 163.504 | 1.00 | 73.78 |
| 3025 | N | ALA | B | 514 | 83.450 | 91.824 | 162.492 | 1.00 | 114.41 |
| 3026 | CA | ALA | B | 514 | 83.868 | 92.174 | 161.147 | 1.00 | 105.99 |
| 3027 | CB | ALA | B | 514 | 82.798 | 91.766 | 160.171 | 1.00 | 110.57 |
| 3028 | C | ALA | B | 514 | 84.167 | 93.657 | 160.992 | 1.00 | 106.98 |
| 3029 | O | ALA | B | 514 | 85.169 | 94.038 | 160.389 | 1.00 | 114.61 |
| 3030 | N | GLU | B | 515 | 83.303 | 94.493 | 161.550 | 1.00 | 107.80 |
| 3031 | CA | GLU | B | 515 | 83.461 | 95.936 | 161.440 | 1.00 | 100.76 |
| 3032 | CB | GLU | B | 515 | 82.162 | 96.623 | 161.855 | 1.00 | 61.09 |
| 3033 | CG | GLU | B | 515 | 81.038 | 96.378 | 160.880 | 1.00 | 142.67 |
| 3034 | CD | GLU | B | 515 | 79.733 | 96.989 | 161.327 | 1.00 | 158.22 |
| 3035 | OE1 | GLU | B | 515 | 79.234 | 96.590 | 162.401 | 1.00 | 121.42 |
| 3036 | OE2 | GLU | B | 515 | 79.210 | 97.862 | 160.600 | 1.00 | 163.79 |
| 3037 | C | GLU | B | 515 | 84.637 | 96.589 | 162.164 | 1.00 | 105.34 |
| 3038 | O | GLU | B | 515 | 85.189 | 97.567 | 161.664 | 1.00 | 78.08 |
| 3039 | N | TRP | B | 516 | 85.034 | 96.082 | 163.327 | 1.00 | 77.37 |
| 3040 | CA | TRP | B | 516 | 86.146 | 96.723 | 164.007 | 1.00 | 80.12 |
| 3041 | CB | TRP | B | 516 | 86.035 | 96.570 | 165.523 | 1.00 | 104.80 |
| 3042 | CG | TRP | B | 516 | 86.442 | 95.275 | 166.113 | 1.00 | 71.05 |
| 3043 | CD2 | TRP | B | 516 | 85.591 | 94.349 | 166.786 | 1.00 | 78.16 |
| 3044 | CE2 | TRP | B | 516 | 86.411 | 93.335 | 167.314 | 1.00 | 93.47 |
| 3045 | CE3 | TRP | B | 516 | 84.211 | 94.281 | 166.999 | 1.00 | 89.55 |
| 3046 | CD1 | TRP | B | 516 | 87.708 | 94.795 | 166.243 | 1.00 | 118.89 |
| 3047 | NE1 | TRP | B | 516 | 87.702 | 93.630 | 166.968 | 1.00 | 138.68 |
| 3048 | CZ2 | TRP | B | 516 | 85.896 | 92.262 | 168.043 | 1.00 | 130.46 |
| 3049 | CZ3 | TRP | B | 516 | 83.698 | 93.218 | 167.722 | 1.00 | 72.54 |
| 3050 | CH2 | TRP | B | 516 | 84.539 | 92.222 | 168.236 | 1.00 | 115.85 |
| 3051 | C | TRP | B | 516 | 87.493 | 96.250 | 163.505 | 1.00 | 126.19 |
| 3052 | O | TRP | B | 516 | 88.524 | 96.836 | 163.833 | 1.00 | 155.29 |
| 3053 | N | GLU | B | 517 | 87.484 | 95.191 | 162.703 | 1.00 | 127.20 |
| 3054 | CA | GLU | B | 517 | 88.715 | 94.681 | 162.120 | 1.00 | 102.94 |
| 3055 | CB | GLU | B | 517 | 88.586 | 93.193 | 161.804 | 1.00 | 124.17 |
| 3056 | CG | GLU | B | 517 | 88.437 | 92.331 | 163.045 | 1.00 | 150.03 |
| 3057 | CD | GLU | B | 517 | 88.603 | 90.854 | 162.756 | 1.00 | 194.54 |
| 3058 | OE1 | GLU | B | 517 | 87.847 | 90.322 | 161.915 | 1.00 | 209.80 |
| 3059 | OE2 | GLU | B | 517 | 89.490 | 90.226 | 163.373 | 1.00 | 178.99 |
| 3060 | C | GLU | B | 517 | 88.923 | 95.495 | 160.848 | 1.00 | 118.06 |
| 3061 | O | GLU | B | 517 | 90.047 | 95.697 | 160.396 | 1.00 | 141.82 |
| 3062 | N | GLN | B | 518 | 87.817 | 95.976 | 160.289 | 1.00 | 96.99 |
| 3063 | CA | GLN | B | 518 | 87.840 | 96.803 | 159.093 | 1.00 | 108.10 |
| 3064 | CB | GLN | B | 518 | 86.407 | 97.051 | 158.611 | 1.00 | 144.13 |
| 3065 | CG | GLN | B | 518 | 86.285 | 97.795 | 157.287 | 1.00 | 173.67 |
| 3066 | CD | GLN | B | 518 | 84.834 | 98.105 | 156.921 | 1.00 | 166.01 |
| 3067 | OE1 | GLN | B | 518 | 83.989 | 97.206 | 156.851 | 1.00 | 119.41 |
| 3068 | NE2 | GLN | B | 518 | 84.544 | 99.382 | 156.686 | 1.00 | 143.37 |
| 3069 | C | GLN | B | 518 | 88.500 | 98.116 | 159.504 | 1.00 | 120.74 |
| 3070 | O | GLN | B | 518 | 89.196 | 98.743 | 158.710 | 1.00 | 92.65 |
| 3071 | N | LYS | B | 519 | 88.260 | 98.509 | 160.758 | 1.00 | 130.00 |
| 3072 | CA | LYS | B | 519 | 88.818 | 99.719 | 161.375 | 1.00 | 130.50 |
| 3073 | CB | LYS | B | 519 | 88.367 | 100.990 | 160.643 | 1.00 | 69.93 |
| 3074 | CG | LYS | B | 519 | 89.179 | 102.223 | 161.053 | 1.00 | 87.64 |
| 3075 | CD | LYS | B | 519 | 89.180 | 103.305 | 159.981 | 1.00 | 120.11 |
| 3076 | CE | LYS | B | 519 | 90.255 | 104.358 | 160.255 | 1.00 | 120.69 |
| 3077 | NZ | LYS | B | 519 | 90.332 | 105.388 | 159.175 | 1.00 | 139.23 |
| 3078 | C | LYS | B | 519 | 88.370 | 99.789 | 162.835 | 1.00 | 113.46 |
| 3079 | O | LYS | B | 519 | 87.399 | 99.145 | 163.209 | 1.00 | 100.53 |
| 3080 | N | ASP | B | 520 | 89.086 | 100.555 | 163.656 | 1.00 | 141.80 |
| 3081 | CA | ASP | B | 520 | 88.752 | 100.712 | 165.075 | 1.00 | 91.69 |
| 3082 | CB | ASP | B | 520 | 90.013 | 100.687 | 165.939 | 1.00 | 139.47 |
| 3083 | CG | ASP | B | 520 | 90.474 | 99.285 | 166.259 | 1.00 | 176.31 |
| 3084 | OD1 | ASP | B | 520 | 89.710 | 98.566 | 166.935 | 1.00 | 155.37 |
| 3085 | OD2 | ASP | B | 520 | 91.593 | 98.907 | 165.843 | 1.00 | 179.10 |
| 3086 | C | ASP | B | 520 | 88.026 | 102.029 | 165.323 | 1.00 | 88.08 |
| 3087 | O | ASP | B | 520 | 87.179 | 102.115 | 166.201 | 1.00 | 108.19 |
| 3088 | N | GLU | B | 521 | 88.367 | 103.055 | 164.552 | 1.00 | 111.16 |
| 3089 | CA | GLU | B | 521 | 87.745 | 104.369 | 164.690 | 1.00 | 96.48 |
| 3090 | CB | GLU | B | 521 | 88.335 | 105.364 | 163.686 | 1.00 | 137.30 |
| 3091 | CG | GLU | B | 521 | 89.404 | 106.294 | 164.229 | 1.00 | 154.75 |
| 3092 | CD | GLU | B | 521 | 89.685 | 107.446 | 163.278 | 1.00 | 162.73 |
| 3093 | OE1 | GLU | B | 521 | 88.761 | 108.254 | 163.051 | 1.00 | 123.78 |
| 3094 | OE2 | GLU | B | 521 | 90.818 | 107.542 | 162.754 | 1.00 | 170.68 |
| 3095 | C | GLU | B | 521 | 86.236 | 104.373 | 164.495 | 1.00 | 94.12 |
| 3096 | O | GLU | B | 521 | 85.750 | 104.213 | 163.377 | 1.00 | 98.75 |
| 3097 | N | PHE | B | 522 | 85.506 | 104.574 | 165.588 | 1.00 | 125.98 |
| 3098 | CA | PHE | B | 522 | 84.052 | 104.669 | 165.563 | 1.00 | 73.40 |
| 3099 | CB | PHE | B | 522 | 83.424 | 103.654 | 166.495 | 1.00 | 46.93 |
| 3100 | CG | PHE | B | 522 | 83.252 | 102.307 | 165.892 | 1.00 | 88.24 |
| 3101 | CD1 | PHE | B | 522 | 84.291 | 101.701 | 165.210 | 1.00 | 107.06 |
| 3102 | CD2 | PHE | B | 522 | 82.051 | 101.625 | 166.026 | 1.00 | 126.15 |
| 3103 | CE1 | PHE | B | 522 | 84.137 | 100.430 | 164.671 | 1.00 | 144.12 |
| 3104 | CE2 | PHE | B | 522 | 81.887 | 100.353 | 165.491 | 1.00 | 86.55 |
| 3105 | CZ | PHE | B | 522 | 82.929 | 99.756 | 164.814 | 1.00 | 98.88 |
| 3106 | C | PHE | B | 522 | 83.737 | 106.065 | 166.062 | 1.00 | 85.06 |
| 3107 | O | PHE | B | 522 | 84.219 | 106.486 | 167.113 | 1.00 | 76.66 |
| 3108 | N | ILE | B | 523 | 82.937 | 106.797 | 165.313 | 1.00 | 64.62 |
| 3109 | CA | ILE | B | 523 | 82.619 | 108.140 | 165.731 | 1.00 | 93.86 |
| 3110 | CB | ILE | B | 523 | 83.006 | 109.136 | 164.638 | 1.00 | 62.16 |
| 3111 | CG2 | ILE | B | 523 | 82.726 | 110.554 | 165.093 | 1.00 | 77.13 |
| 3112 | CG1 | ILE | B | 523 | 84.483 | 108.963 | 164.309 | 1.00 | 68.68 |
| 3113 | CD1 | ILE | B | 523 | 85.004 | 109.946 | 163.268 | 1.00 | 144.70 |
| 3114 | C | ILE | B | 523 | 81.152 | 108.304 | 166.076 | 1.00 | 89.50 |
| 3115 | O | ILE | B | 523 | 80.276 | 107.800 | 165.371 | 1.00 | 97.40 |
| 3116 | N | CYS | B | 524 | 80.903 | 108.998 | 167.183 | 1.00 | 89.54 |
| 3117 | CA | CYS | B | 524 | 79.551 | 109.286 | 167.642 | 1.00 | 88.30 |
| 3118 | C | CYS | B | 524 | 79.358 | 110.792 | 167.558 | 1.00 | 53.16 |
| 3119 | O | CYS | B | 524 | 79.589 | 111.496 | 168.514 | 1.00 | 59.51 |
| 3120 | CB | CYS | B | 524 | 79.363 | 108.835 | 169.082 | 1.00 | 70.18 |
| 3121 | SG | CYS | B | 524 | 77.896 | 109.575 | 169.868 | 1.00 | 102.40 |
| 3122 | N | ARG | B | 525 | 78.939 | 111.272 | 166.397 | 1.00 | 85.33 |
| 3123 | CA | ARG | B | 525 | 78.740 | 112.694 | 166.166 | 1.00 | 42.18 |
| 3124 | CB | ARG | B | 525 | 78.613 | 112.956 | 164.664 | 1.00 | 51.31 |
| 3125 | CG | ARG | B | 525 | 78.750 | 114.395 | 164.270 | 1.00 | 55.59 |
| 3126 | CD | ARG | B | 525 | 79.294 | 114.553 | 162.852 | 1.00 | 75.11 |
| 3127 | NE | ARG | B | 525 | 78.328 | 114.264 | 161.795 | 1.00 | 77.64 |
| 3128 | CZ | ARG | B | 525 | 78.518 | 114.587 | 160.517 | 1.00 | 147.54 |
| 3129 | NH1 | ARG | B | 525 | 79.633 | 115.211 | 160.143 | 1.00 | 139.91 |
| 3130 | NH2 | ARG | B | 525 | 77.598 | 114.280 | 159.610 | 1.00 | 128.34 |
| 3131 | C | ARG | B | 525 | 77.501 | 113.197 | 166.880 | 1.00 | 81.14 |
| 3132 | O | ARG | B | 525 | 76.714 | 112.411 | 167.406 | 1.00 | 77.17 |
| 3133 | N | ALA | B | 526 | 77.344 | 114.517 | 166.897 | 1.00 | 86.44 |
| 3134 | CA | ALA | B | 526 | 76.209 | 115.177 | 167.538 | 1.00 | 64.44 |
| 3135 | CB | ALA | B | 526 | 76.469 | 115.358 | 169.025 | 1.00 | 40.12 |
| 3136 | C | ALA | B | 526 | 76.003 | 116.527 | 166.881 | 1.00 | 64.81 |
| 3137 | O | ALA | B | 526 | 76.957 | 117.256 | 166.609 | 1.00 | 75.52 |
| 3138 | N | VAL | B | 527 | 74.753 | 116.858 | 166.616 | 1.00 | 45.76 |
| 3139 | CA | VAL | B | 527 | 74.446 | 118.123 | 165.984 | 1.00 | 48.87 |
| 3140 | CB | VAL | B | 527 | 73.702 | 117.900 | 164.683 | 1.00 | 72.30 |
| 3141 | CG1 | VAL | B | 527 | 73.434 | 119.222 | 164.005 | 1.00 | 65.22 |
| 3142 | CG2 | VAL | B | 527 | 74.512 | 116.984 | 163.798 | 1.00 | 69.67 |
| 3143 | C | VAL | B | 527 | 73.583 | 118.949 | 166.912 | 1.00 | 62.63 |
| 3144 | O | VAL | B | 527 | 72.494 | 118.525 | 167.296 | 1.00 | 107.55 |
| 3145 | N | HIS | B | 528 | 74.075 | 120.123 | 167.290 | 1.00 | 88.39 |
| 3146 | CA | HIS | B | 528 | 73.324 | 121.009 | 168.177 | 1.00 | 72.81 |
| 3147 | CB | HIS | B | 528 | 73.883 | 120.954 | 169.599 | 1.00 | 43.78 |
| 3148 | CG | HIS | B | 528 | 73.000 | 121.603 | 170.619 | 1.00 | 68.69 |
| 3149 | CD2 | HIS | B | 528 | 72.544 | 122.873 | 170.730 | 1.00 | 102.41 |
| 3150 | ND1 | HIS | B | 528 | 72.530 | 120.932 | 171.727 | 1.00 | 114.24 |
| 3151 | CE1 | HIS | B | 528 | 71.828 | 121.761 | 172.479 | 1.00 | 111.59 |
| 3152 | NE2 | HIS | B | 528 | 71.822 | 122.946 | 171.896 | 1.00 | 127.17 |
| 3153 | C | HIS | B | 528 | 73.405 | 122.421 | 167.644 | 1.00 | 69.99 |
| 3154 | O | HIS | B | 528 | 74.322 | 122.745 | 166.877 | 1.00 | 80.61 |
| 3155 | N | GLU | B | 529 | 72.439 | 123.246 | 168.040 | 1.00 | 80.81 |
| 3156 | CA | GLU | B | 529 | 72.388 | 124.630 | 167.603 | 1.00 | 110.94 |
| 3157 | CB | GLU | B | 529 | 71.056 | 125.236 | 168.007 | 1.00 | 143.10 |
| 3158 | CG | GLU | B | 529 | 70.885 | 126.632 | 167.550 | 1.00 | 190.23 |
| 3159 | CD | GLU | B | 529 | 69.576 | 127.233 | 168.045 | 1.00 | 209.79 |
| 3160 | OE1 | GLU | B | 529 | 69.275 | 128.356 | 168.100 | 1.00 | 211.79 |
| 3161 | OE2 | GLU | B | 529 | 68.633 | 126.689 | 168.465 | 1.00 | 204.23 |
| 3162 | C | GLU | B | 529 | 73.552 | 125.453 | 168.173 | 1.00 | 115.08 |
| 3163 | O | GLU | B | 529 | 73.915 | 126.492 | 167.625 | 1.00 | 93.09 |
| 3164 | N | ALA | B | 530 | 74.184 | 124.942 | 169.228 | 1.00 | 104.56 |
| 3165 | CA | ALA | B | 530 | 75.301 | 125.633 | 169.882 | 1.00 | 119.10 |
| 3166 | CB | ALA | B | 530 | 75.312 | 125.312 | 171.383 | 1.00 | 102.81 |
| 3167 | C | ALA | B | 530 | 76.657 | 125.298 | 169.285 | 1.00 | 143.44 |
| 3168 | O | ALA | B | 530 | 77.105 | 125.948 | 168.341 | 1.00 | 167.11 |
| 3169 | N | ALA | B | 531 | 77.296 | 124.287 | 169.868 | 1.00 | 133.04 |
| 3170 | CA | ALA | B | 531 | 78.608 | 123.796 | 169.457 | 1.00 | 161.80 |
| 3171 | CB | ALA | B | 531 | 78.530 | 122.299 | 169.233 | 1.00 | 131.50 |
| 3172 | C | ALA | B | 531 | 79.240 | 124.461 | 168.235 | 1.00 | 183.35 |
| 3173 | O | ALA | B | 531 | 78.602 | 124.634 | 167.193 | 1.00 | 180.45 |
| 3174 | N | SER | B | 532 | 80.513 | 124.816 | 168.369 | 1.00 | 186.13 |
| 3175 | CA | SER | B | 532 | 81.245 | 125.437 | 167.279 | 1.00 | 159.85 |
| 3176 | CB | SER | B | 532 | 81.945 | 126.707 | 167.764 | 1.00 | 149.12 |
| 3177 | OG | SER | B | 532 | 81.012 | 127.751 | 167.971 | 1.00 | 131.01 |
| 3178 | C | SER | B | 532 | 82.270 | 124.459 | 166.721 | 1.00 | 143.48 |
| 3179 | O | SER | B | 532 | 82.606 | 123.460 | 167.359 | 1.00 | 131.10 |
| 3180 | N | PRO | B | 533 | 82.789 | 124.741 | 165.522 | 1.00 | 133.12 |
| 3181 | CD | PRO | B | 533 | 84.018 | 124.105 | 165.014 | 1.00 | 145.42 |
| 3182 | CA | PRO | B | 533 | 82.446 | 125.915 | 164.718 | 1.00 | 114.44 |
| 3183 | CB | PRO | B | 533 | 83.793 | 126.336 | 164.170 | 1.00 | 151.25 |
| 3184 | CG | PRO | B | 533 | 84.400 | 124.997 | 163.830 | 1.00 | 156.89 |
| 3185 | C | PRO | B | 533 | 81.477 | 125.558 | 163.605 | 1.00 | 132.46 |
| 3186 | O | PRO | B | 533 | 80.902 | 126.440 | 162.963 | 1.00 | 100.35 |
| 3187 | N | SER | B | 534 | 81.320 | 124.256 | 163.383 | 1.00 | 143.66 |
| 3188 | CA | SER | B | 534 | 80.452 | 123.734 | 162.335 | 1.00 | 132.97 |
| 3189 | CB | SER | B | 534 | 81.191 | 122.649 | 161.552 | 1.00 | 161.58 |
| 3190 | OG | SER | B | 534 | 81.699 | 121.659 | 162.433 | 1.00 | 165.58 |
| 3191 | C | SER | B | 534 | 79.150 | 123.167 | 162.880 | 1.00 | 119.62 |
| 3192 | O | SER | B | 534 | 78.478 | 122.386 | 162.208 | 1.00 | 111.21 |
| 3193 | N | GLN | B | 535 | 78.797 | 123.562 | 164.097 | 1.00 | 121.56 |
| 3194 | CA | GLN | B | 535 | 77.567 | 123.100 | 164.729 | 1.00 | 104.19 |
| 3195 | CB | GLN | B | 535 | 76.364 | 123.514 | 163.874 | 1.00 | 39.24 |
| 3196 | CG | GLN | B | 535 | 76.251 | 125.023 | 163.741 | 1.00 | 73.06 |
| 3197 | CD | GLN | B | 535 | 76.192 | 125.485 | 162.297 | 1.00 | 105.92 |
| 3198 | OE1 | GLN | B | 535 | 76.935 | 124.993 | 161.445 | 1.00 | 122.35 |
| 3199 | NE2 | GLN | B | 535 | 75.317 | 126.449 | 162.017 | 1.00 | 116.95 |
| 3200 | C | GLN | B | 535 | 77.588 | 121.590 | 164.945 | 1.00 | 81.66 |
| 3201 | O | GLN | B | 535 | 76.565 | 120.977 | 165.244 | 1.00 | 75.14 |
| 3202 | N | THR | B | 536 | 78.772 | 121.005 | 164.809 | 1.00 | 75.59 |
| 3203 | CA | THR | B | 536 | 78.960 | 119.571 | 164.983 | 1.00 | 73.02 |
| 3204 | CB | THR | B | 536 | 79.677 | 118.967 | 163.771 | 1.00 | 96.53 |
| 3205 | OG1 | THR | B | 536 | 78.861 | 119.117 | 162.604 | 1.00 | 138.70 |
| 3206 | CG2 | THR | B | 536 | 79.973 | 117.509 | 164.005 | 1.00 | 87.18 |
| 3207 | C | THR | B | 536 | 79.825 | 119.321 | 166.200 | 1.00 | 72.59 |
| 3208 | O | THR | B | 536 | 80.514 | 120.215 | 166.672 | 1.00 | 108.65 |
| 3209 | N | VAL | B | 537 | 79.798 | 118.095 | 166.694 | 1.00 | 58.96 |
| 3210 | CA | VAL | B | 537 | 80.600 | 117.708 | 167.847 | 1.00 | 83.66 |
| 3211 | CB | VAL | B | 537 | 79.969 | 118.190 | 169.146 | 1.00 | 52.09 |
| 3212 | CG1 | VAL | B | 537 | 80.474 | 117.351 | 170.311 | 1.00 | 68.77 |
| 3213 | CG2 | VAL | B | 537 | 80.317 | 119.645 | 169.366 | 1.00 | 119.61 |
| 3214 | C | VAL | B | 537 | 80.723 | 116.195 | 167.904 | 1.00 | 99.63 |
| 3215 | O | VAL | B | 537 | 79.719 | 115.495 | 167.960 | 1.00 | 109.69 |
| 3216 | N | GLN | B | 538 | 81.947 | 115.685 | 167.904 | 1.00 | 100.40 |
| 3217 | CA | GLN | B | 538 | 82.128 | 114.242 | 167.931 | 1.00 | 90.82 |
| 3218 | CB | GLN | B | 538 | 82.389 | 113.741 | 166.508 | 1.00 | 92.68 |
| 3219 | CG | GLN | B | 538 | 83.519 | 114.462 | 165.790 | 1.00 | 69.71 |
| 3220 | CD | GLN | B | 538 | 83.524 | 114.199 | 164.292 | 1.00 | 97.76 |
| 3221 | OE1 | GLN | B | 538 | 82.734 | 114.776 | 163.543 | 1.00 | 78.04 |
| 3222 | NE2 | GLN | B | 538 | 84.410 | 113.314 | 163.850 | 1.00 | 116.83 |
| 3223 | C | GLN | B | 538 | 83.238 | 113.781 | 168.859 | 1.00 | 75.38 |
| 3224 | O | GLN | B | 538 | 84.090 | 114.568 | 169.262 | 1.00 | 96.22 |
| 3225 | N | ARG | B | 539 | 83.206 | 112.500 | 169.206 | 1.00 | 51.75 |
| 3226 | CA | ARG | B | 539 | 84.215 | 111.907 | 170.074 | 1.00 | 99.09 |
| 3227 | CB | ARG | B | 539 | 83.719 | 111.803 | 171.517 | 1.00 | 100.20 |
| 3228 | CG | ARG | B | 539 | 84.820 | 111.481 | 172.533 | 1.00 | 145.36 |
| 3229 | CD | ARG | B | 539 | 85.250 | 112.726 | 173.313 | 1.00 | 159.80 |
| 3230 | NE | ARG | B | 539 | 85.363 | 113.903 | 172.451 | 1.00 | 167.91 |
| 3231 | CZ | ARG | B | 539 | 85.663 | 115.127 | 172.880 | 1.00 | 150.28 |
| 3232 | NH1 | ARG | B | 539 | 85.887 | 115.344 | 174.170 | 1.00 | 164.72 |
| 3233 | NH2 | ARG | B | 539 | 85.726 | 116.136 | 172.017 | 1.00 | 114.07 |
| 3234 | C | ARG | B | 539 | 84.493 | 110.512 | 169.547 | 1.00 | 119.86 |
| 3235 | O | ARG | B | 539 | 83.580 | 109.700 | 169.426 | 1.00 | 90.13 |
| 3236 | N | ALA | B | 540 | 85.753 | 110.236 | 169.234 | 1.00 | 128.12 |
| 3237 | CA | ALA | B | 540 | 86.136 | 108.933 | 168.712 | 1.00 | 89.16 |
| 3238 | CB | ALA | B | 540 | 87.531 | 109.015 | 168.111 | 1.00 | 124.98 |
| 3239 | C | ALA | B | 540 | 86.100 | 107.866 | 169.796 | 1.00 | 76.22 |
| 3240 | O | ALA | B | 540 | 86.009 | 108.171 | 170.980 | 1.00 | 109.84 |
| 3241 | N | VAL | B | 541 | 86.173 | 106.612 | 169.376 | 1.00 | 60.52 |
| 3242 | CA | VAL | B | 541 | 86.179 | 105.491 | 170.303 | 1.00 | 86.69 |
| 3243 | CB | VAL | B | 541 | 84.770 | 105.139 | 170.724 | 1.00 | 87.58 |
| 3244 | CG1 | VAL | B | 541 | 84.005 | 104.682 | 169.512 | 1.00 | 77.89 |
| 3245 | CG2 | VAL | B | 541 | 84.785 | 104.059 | 171.807 | 1.00 | 52.44 |
| 3246 | C | VAL | B | 541 | 86.786 | 104.283 | 169.597 | 1.00 | 102.20 |
| 3247 | O | VAL | B | 541 | 87.163 | 104.375 | 168.430 | 1.00 | 152.01 |
| 3248 | N | SER | B | 542 | 86.882 | 103.159 | 170.306 | 1.00 | 105.84 |
| 3249 | CA | SER | B | 542 | 87.426 | 101.922 | 169.748 | 1.00 | 89.13 |
| 3250 | CB | SER | B | 542 | 88.722 | 102.199 | 168.975 | 1.00 | 113.60 |
| 3251 | OG | SER | B | 542 | 89.682 | 102.852 | 169.791 | 1.00 | 119.14 |
| 3252 | C | SER | B | 542 | 87.713 | 100.893 | 170.829 | 1.00 | 69.68 |
| 3253 | O | SER | B | 542 | 87.935 | 101.248 | 171.987 | 1.00 | 134.26 |
| 3254 | N | VAL | B | 543 | 87.710 | 99.618 | 170.452 | 1.00 | 64.03 |
| 3255 | CA | VAL | B | 543 | 88.017 | 98.550 | 171.401 | 1.00 | 113.44 |
| 3256 | CB | VAL | B | 543 | 87.339 | 97.221 | 171.023 | 1.00 | 126.85 |
| 3257 | CG1 | VAL | B | 543 | 87.101 | 96.404 | 172.285 | 1.00 | 62.70 |
| 3258 | CG2 | VAL | B | 543 | 86.055 | 97.470 | 170.223 | 1.00 | 22.03 |
| 3259 | C | VAL | B | 543 | 89.530 | 98.308 | 171.372 | 1.00 | 152.87 |
| 3260 | O | VAL | B | 543 | 90.183 | 98.571 | 170.361 | 1.00 | 170.87 |
| 3261 | N | ASN | B | 544 | 90.079 | 97.799 | 172.472 | 1.00 | 147.76 |
| 3262 | CA | ASN | B | 544 | 91.511 | 97.522 | 172.561 | 1.00 | 158.75 |
| 3263 | CB | ASN | B | 544 | 91.944 | 96.576 | 171.431 | 1.00 | 162.14 |
| 3264 | CG | ASN | B | 544 | 91.163 | 95.267 | 171.423 | 1.00 | 149.81 |
| 3265 | OD1 | ASN | B | 544 | 91.128 | 94.541 | 172.417 | 1.00 | 152.63 |
| 3266 | ND2 | ASN | B | 544 | 90.540 | 94.958 | 170.289 | 1.00 | 96.95 |
| 3267 | C | ASN | B | 544 | 92.336 | 98.814 | 172.498 | 1.00 | 165.23 |
| 3268 | O | ASN | B | 544 | 93.135 | 99.041 | 173.435 | 1.00 | 169.09 |
| 3269 | OXT | ASN | B | 544 | 92.179 | 99.582 | 171.517 | 1.00 | 91.11 |
| 3270 | C1 | NAG | B | 694 | 43.351 | 106.499 | 163.692 | 1.00 | 45.89 |
| 3271 | C2 | NAG | B | 694 | 43.324 | 107.210 | 165.050 | 1.00 | 60.23 |
| 3272 | N2 | NAG | B | 694 | 42.009 | 107.764 | 165.311 | 1.00 | 56.36 |
| 3273 | C7 | NAG | B | 694 | 41.107 | 107.072 | 166.005 | 1.00 | 75.39 |
| 3274 | O7 | NAG | B | 694 | 41.224 | 105.878 | 166.283 | 1.00 | 71.41 |
| 3275 | C8 | NAG | B | 694 | 39.876 | 107.819 | 166.449 | 1.00 | 22.03 |
| 3276 | C3 | NAG | B | 694 | 44.367 | 108.326 | 165.119 | 1.00 | 58.80 |
| 3277 | O3 | NAG | B | 694 | 44.468 | 108.774 | 166.459 | 1.00 | 74.72 |
| 3278 | C4 | NAG | B | 694 | 45.745 | 107.860 | 164.653 | 1.00 | 56.10 |
| 3279 | O4 | NAG | B | 694 | 46.595 | 109.009 | 164.472 | 1.00 | 83.05 |
| 3280 | C5 | NAG | B | 694 | 45.633 | 107.121 | 163.324 | 1.00 | 25.95 |
| 3281 | O5 | NAG | B | 694 | 44.683 | 106.052 | 163.418 | 1.00 | 53.24 |
| 3282 | C6 | NAG | B | 694 | 46.944 | 106.492 | 162.896 | 1.00 | 119.03 |
| 3283 | O6 | NAG | B | 694 | 46.718 | 105.307 | 162.144 | 1.00 | 138.62 |
| 3284 | C1 | NAG | B | 695 | 47.667 | 109.149 | 165.334 | 1.00 | 105.64 |
| 3285 | C2 | NAG | B | 695 | 48.912 | 109.587 | 164.537 | 1.00 | 59.77 |
| 3286 | N2 | NAG | B | 695 | 49.357 | 108.488 | 163.700 | 1.00 | 93.15 |
| 3287 | C7 | NAG | B | 695 | 49.909 | 108.731 | 162.516 | 1.00 | 103.18 |
| 3288 | O7 | NAG | B | 695 | 51.131 | 108.754 | 162.340 | 1.00 | 136.64 |
| 3289 | C8 | NAG | B | 695 | 48.960 | 108.985 | 161.349 | 1.00 | 60.78 |
| 3290 | C3 | NAG | B | 695 | 50.062 | 110.046 | 165.456 | 1.00 | 79.33 |
| 3291 | O3 | NAG | B | 695 | 51.051 | 110.680 | 164.656 | 1.00 | 98.62 |
| 3292 | C4 | NAG | B | 695 | 49.530 | 111.028 | 166.523 | 1.00 | 100.15 |
| 3293 | O4 | NAG | B | 695 | 50.546 | 111.348 | 167.496 | 1.00 | 82.39 |
| 3294 | C5 | NAG | B | 695 | 48.357 | 110.377 | 167.236 | 1.00 | 125.01 |
| 3295 | O5 | NAG | B | 695 | 47.306 | 110.143 | 166.294 | 1.00 | 76.78 |
| 3296 | C6 | NAG | B | 695 | 47.797 | 111.249 | 168.331 | 1.00 | 149.78 |
| 3297 | O6 | NAG | B | 695 | 46.422 | 110.979 | 168.540 | 1.00 | 106.46 |
| 3298 | C1 | MAN | B | 696 | 51.371 | 112.429 | 167.246 | 1.00 | 73.26 |
| 3299 | C2 | MAN | B | 696 | 51.896 | 112.953 | 168.555 | 1.00 | 85.84 |
| 3300 | O2 | MAN | B | 696 | 52.572 | 111.906 | 169.203 | 1.00 | 84.25 |
| 3301 | C3 | MAN | B | 696 | 52.858 | 114.113 | 168.311 | 1.00 | 120.67 |
| 3302 | O3 | MAN | B | 696 | 53.494 | 114.526 | 169.543 | 1.00 | 161.15 |
| 3303 | C4 | MAN | B | 696 | 53.939 | 113.688 | 167.318 | 1.00 | 142.38 |
| 3304 | O4 | MAN | B | 696 | 54.644 | 114.828 | 166.907 | 1.00 | 158.25 |
| 3305 | C5 | MAN | B | 696 | 53.376 | 112.998 | 166.077 | 1.00 | 122.76 |
| 3306 | O5 | MAN | B | 696 | 52.478 | 111.940 | 166.480 | 1.00 | 129.27 |
| 3307 | C6 | MAN | B | 696 | 54.495 | 112.437 | 165.214 | 1.00 | 121.56 |
| 3308 | O6 | MAN | B | 696 | 54.271 | 111.063 | 164.855 | 1.00 | 154.87 |
| 3309 | C1 | MAN | B | 697 | 55.439 | 110.276 | 165.023 | 1.00 | 145.40 |
| 3310 | C2 | MAN | B | 697 | 56.117 | 110.375 | 166.417 | 1.00 | 145.77 |
| 3311 | O2 | MAN | B | 697 | 57.047 | 109.270 | 166.517 | 1.00 | 146.40 |
| 3312 | C3 | MAN | B | 697 | 56.937 | 111.658 | 166.557 | 1.00 | 141.88 |
| 3313 | O3 | MAN | B | 697 | 57.844 | 111.476 | 167.629 | 1.00 | 137.62 |
| 3314 | C4 | MAN | B | 697 | 57.782 | 111.951 | 165.323 | 1.00 | 141.83 |
| 3315 | O4 | MAN | B | 697 | 58.361 | 113.247 | 165.387 | 1.00 | 139.00 |
| 3316 | C5 | MAN | B | 697 | 57.018 | 111.797 | 164.042 | 1.00 | 143.82 |
| 3317 | O5 | MAN | B | 697 | 56.422 | 110.499 | 164.016 | 1.00 | 146.27 |
| 3318 | C6 | MAN | B | 697 | 57.919 | 111.862 | 162.860 | 1.00 | 148.17 |
| 3319 | O6 | MAN | B | 697 | 57.262 | 111.446 | 161.673 | 1.00 | 150.80 |
| 3320 | C1 | MAN | B | 698 | 52.693 | 115.215 | 170.457 | 1.00 | 166.73 |
| 3321 | C2 | MAN | B | 698 | 53.464 | 116.407 | 171.056 | 1.00 | 180.10 |
| 3322 | O2 | MAN | B | 698 | 52.557 | 117.261 | 171.748 | 1.00 | 145.07 |
| 3323 | C3 | MAN | B | 698 | 54.563 | 115.916 | 172.016 | 1.00 | 172.30 |
| 3324 | O3 | MAN | B | 698 | 55.188 | 117.024 | 172.657 | 1.00 | 137.27 |
| 3325 | C4 | MAN | B | 698 | 53.964 | 114.962 | 173.060 | 1.00 | 164.34 |
| 3326 | O4 | MAN | B | 698 | 54.992 | 114.436 | 173.885 | 1.00 | 140.37 |
| 3327 | C5 | MAN | B | 698 | 53.231 | 113.819 | 172.351 | 1.00 | 150.05 |
| 3328 | O5 | MAN | B | 698 | 52.208 | 114.363 | 171.491 | 1.00 | 142.54 |
| 3329 | C6 | MAN | B | 698 | 52.553 | 112.858 | 173.311 | 1.00 | 146.09 |
| 3330 | O6 | MAN | B | 698 | 51.158 | 112.769 | 173.056 | 1.00 | 158.71 |
| 3331 | C1 | MAN | B | 699 | 56.966 | 108.431 | 167.643 | 1.00 | 147.68 |
| 3332 | C2 | MAN | B | 699 | 58.038 | 108.848 | 168.676 | 1.00 | 149.11 |
| 3333 | O2 | MAN | B | 699 | 57.797 | 108.220 | 169.928 | 1.00 | 144.31 |
| 3334 | C3 | MAN | B | 699 | 59.464 | 108.512 | 168.172 | 1.00 | 149.80 |
| 3335 | O3 | MAN | B | 699 | 60.421 | 108.765 | 169.197 | 1.00 | 142.63 |
| 3336 | C4 | MAN | B | 699 | 59.578 | 107.043 | 167.743 | 1.00 | 151.96 |
| 3337 | O4 | MAN | B | 699 | 60.851 | 106.815 | 167.127 | 1.00 | 149.52 |
| 3338 | C5 | MAN | B | 699 | 58.427 | 106.645 | 166.780 | 1.00 | 152.87 |
| 3339 | O5 | MAN | B | 699 | 57.110 | 107.042 | 167.306 | 1.00 | 150.31 |
| 3340 | C6 | MAN | B | 699 | 58.378 | 105.138 | 166.486 | 1.00 | 152.00 |
| 3341 | O6 | MAN | B | 699 | 58.826 | 104.348 | 167.584 | 1.00 | 155.30 |
| 3342 | C | CYS | D | 329 | 40.977 | 121.748 | 178.634 | 1.00 | 210.29 |
| 3343 | O | CYS | D | 329 | 41.782 | 122.273 | 179.404 | 1.00 | 189.25 |
| 3344 | CB | CYS | D | 329 | 41.494 | 121.206 | 176.231 | 1.00 | 224.93 |
| 3345 | SG | CYS | D | 329 | 39.884 | 121.730 | 175.555 | 1.00 | 250.42 |
| 3346 | N | CYS | D | 329 | 40.558 | 119.454 | 177.703 | 1.00 | 206.24 |
| 3347 | CA | CYS | D | 329 | 41.438 | 120.654 | 177.662 | 1.00 | 208.57 |
| 3348 | N | ASP | D | 330 | 39.687 | 122.084 | 178.603 | 1.00 | 212.05 |
| 3349 | CA | ASP | D | 330 | 39.131 | 123.119 | 179.482 | 1.00 | 192.97 |
| 3350 | CB | ASP | D | 330 | 38.840 | 124.395 | 178.679 | 1.00 | 200.39 |
| 3351 | CG | ASP | D | 330 | 38.321 | 125.535 | 179.549 | 1.00 | 207.96 |
| 3352 | OD1 | ASP | D | 330 | 37.203 | 125.420 | 180.100 | 1.00 | 182.28 |
| 3353 | OD2 | ASP | D | 330 | 39.037 | 126.552 | 179.681 | 1.00 | 213.37 |
| 3354 | C | ASP | D | 330 | 37.854 | 122.657 | 180.187 | 1.00 | 188.18 |
| 3355 | O | ASP | D | 330 | 37.908 | 122.095 | 181.282 | 1.00 | 170.40 |
| 3356 | N | SER | D | 331 | 36.707 | 122.905 | 179.557 | 1.00 | 186.14 |
| 3357 | CA | SER | D | 331 | 35.419 | 122.512 | 180.123 | 1.00 | 155.83 |
| 3358 | CB | SER | D | 331 | 34.266 | 123.184 | 179.364 | 1.00 | 152.11 |
| 3359 | OG | SER | D | 331 | 34.055 | 122.582 | 178.092 | 1.00 | 95.70 |
| 3360 | C | SER | D | 331 | 35.242 | 120.995 | 180.068 | 1.00 | 163.80 |
| 3361 | O | SER | D | 331 | 34.998 | 120.366 | 181.098 | 1.00 | 161.57 |
| 3362 | N | ASN | D | 332 | 35.368 | 120.419 | 178.869 | 1.00 | 159.48 |
| 3363 | CA | ASN | D | 332 | 35.214 | 118.978 | 178.678 | 1.00 | 122.47 |
| 3364 | CB | ASN | D | 332 | 35.786 | 118.530 | 177.325 | 1.00 | 172.30 |
| 3365 | CG | ASN | D | 332 | 37.269 | 118.836 | 177.170 | 1.00 | 223.31 |
| 3366 | OD1 | ASN | D | 332 | 37.916 | 119.341 | 178.087 | 1.00 | 239.48 |
| 3367 | ND2 | ASN | D | 332 | 37.816 | 118.520 | 175.998 | 1.00 | 237.24 |
| 3368 | C | ASN | D | 332 | 35.874 | 118.205 | 179.816 | 1.00 | 105.80 |
| 3369 | O | ASN | D | 332 | 37.091 | 118.207 | 179.975 | 1.00 | 93.15 |
| 3370 | N | PRO | D | 333 | 35.064 | 117.539 | 180.644 | 1.00 | 64.26 |
| 3371 | CD | PRO | D | 333 | 33.600 | 117.604 | 180.724 | 1.00 | 41.20 |
| 3372 | CA | PRO | D | 333 | 35.595 | 116.778 | 181.771 | 1.00 | 41.52 |
| 3373 | CB | PRO | D | 333 | 34.363 | 116.548 | 182.633 | 1.00 | 35.93 |
| 3374 | CG | PRO | D | 333 | 33.402 | 117.622 | 182.204 | 1.00 | 77.83 |
| 3375 | C | PRO | D | 333 | 36.222 | 115.480 | 181.314 | 1.00 | 54.76 |
| 3376 | O | PRO | D | 333 | 35.858 | 114.951 | 180.272 | 1.00 | 34.59 |
| 3377 | N | ARG | D | 334 | 37.160 | 114.976 | 182.107 | 1.00 | 19.96 |
| 3378 | CA | ARG | D | 334 | 37.866 | 113.731 | 181.813 | 1.00 | 40.10 |
| 3379 | CB | ARG | D | 334 | 39.389 | 113.936 | 181.935 | 1.00 | 60.92 |
| 3380 | CG | ARG | D | 334 | 40.245 | 112.697 | 181.633 | 1.00 | 28.56 |
| 3381 | CD | ARG | D | 334 | 41.745 | 112.986 | 181.457 | 1.00 | 78.41 |
| 3382 | NE | ARG | D | 334 | 42.377 | 112.028 | 180.545 | 1.00 | 73.15 |
| 3383 | CZ | ARG | D | 334 | 42.757 | 112.326 | 179.306 | 1.00 | 100.07 |
| 3384 | NH1 | ARG | D | 334 | 42.575 | 113.553 | 178.841 | 1.00 | 59.54 |
| 3385 | NH2 | ARG | D | 334 | 43.299 | 111.401 | 178.525 | 1.00 | 145.40 |
| 3386 | C | ARG | D | 334 | 37.378 | 112.727 | 182.841 | 1.00 | 5.42 |
| 3387 | O | ARG | D | 334 | 37.095 | 113.087 | 183.979 | 1.00 | 48.58 |
| 3388 | N | GLY | D | 335 | 37.273 | 111.473 | 182.444 | 1.00 | 20.86 |
| 3389 | CA | GLY | D | 335 | 36.759 | 110.464 | 183.343 | 1.00 | 41.07 |
| 3390 | C | GLY | D | 335 | 37.790 | 109.753 | 184.165 | 1.00 | 29.22 |
| 3391 | O | GLY | D | 335 | 38.979 | 109.833 | 183.880 | 1.00 | 46.30 |
| 3392 | N | VAL | D | 336 | 37.326 | 109.034 | 185.179 | 1.00 | 37.28 |
| 3393 | CA | VAL | D | 336 | 38.220 | 108.307 | 186.063 | 1.00 | 5.96 |
| 3394 | CB | VAL | D | 336 | 37.470 | 107.553 | 187.153 | 1.00 | 31.42 |
| 3395 | CG1 | VAL | D | 336 | 38.326 | 106.455 | 187.693 | 1.00 | 28.30 |
| 3396 | CG2 | VAL | D | 336 | 37.136 | 108.471 | 188.264 | 1.00 | 26.00 |
| 3397 | C | VAL | D | 336 | 39.053 | 107.302 | 185.328 | 1.00 | 38.88 |
| 3398 | O | VAL | D | 336 | 38.554 | 106.584 | 184.463 | 1.00 | 39.16 |
| 3399 | N | SER | D | 337 | 40.325 | 107.252 | 185.712 | 1.00 | 43.15 |
| 3400 | CA | SER | D | 337 | 41.306 | 106.342 | 185.141 | 1.00 | 54.37 |
| 3401 | CB | SER | D | 337 | 42.393 | 107.149 | 184.445 | 1.00 | 48.14 |
| 3402 | OG | SER | D | 337 | 41.836 | 108.240 | 183.724 | 1.00 | 87.47 |
| 3403 | C | SER | D | 337 | 41.904 | 105.601 | 186.321 | 1.00 | 32.96 |
| 3404 | O | SER | D | 337 | 41.957 | 106.143 | 187.413 | 1.00 | 38.71 |
| 3405 | N | ALA | D | 338 | 42.330 | 104.361 | 186.128 | 1.00 | 43.73 |
| 3406 | CA | ALA | D | 338 | 42.942 | 103.622 | 187.234 | 1.00 | 32.03 |
| 3407 | CB | ALA | D | 338 | 42.005 | 102.619 | 187.813 | 1.00 | 13.06 |
| 3408 | C | ALA | D | 338 | 44.176 | 102.928 | 186.739 | 1.00 | 38.96 |
| 3409 | O | ALA | D | 338 | 44.243 | 102.514 | 185.591 | 1.00 | 58.49 |
| 3410 | N | TYR | D | 339 | 45.148 | 102.798 | 187.626 | 1.00 | 53.04 |
| 3411 | CA | TYR | D | 339 | 46.417 | 102.195 | 187.298 | 1.00 | 41.03 |
| 3412 | CB | TYR | D | 339 | 47.422 | 103.306 | 187.101 | 1.00 | 30.65 |
| 3413 | CG | TYR | D | 339 | 46.976 | 104.376 | 186.120 | 1.00 | 5.42 |
| 3414 | CD1 | TYR | D | 339 | 46.755 | 105.677 | 186.536 | 1.00 | 64.60 |
| 3415 | CE1 | TYR | D | 339 | 46.456 | 106.687 | 185.625 | 1.00 | 68.13 |
| 3416 | CD2 | TYR | D | 339 | 46.873 | 104.104 | 184.765 | 1.00 | 66.56 |
| 3417 | CE2 | TYR | D | 339 | 46.575 | 105.104 | 183.851 | 1.00 | 42.43 |
| 3418 | CZ | TYR | D | 339 | 46.373 | 106.396 | 184.287 | 1.00 | 52.17 |
| 3419 | OH | TYR | D | 339 | 46.134 | 107.408 | 183.379 | 1.00 | 96.24 |
| 3420 | C | TYR | D | 339 | 46.835 | 101.285 | 188.439 | 1.00 | 48.87 |
| 3421 | O | TYR | D | 339 | 46.362 | 101.445 | 189.558 | 1.00 | 76.65 |
| 3422 | N | LEU | D | 340 | 47.730 | 100.345 | 188.175 | 1.00 | 64.84 |
| 3423 | CA | LEU | D | 340 | 48.140 | 99.410 | 189.213 | 1.00 | 61.58 |
| 3424 | CB | LEU | D | 340 | 47.263 | 98.177 | 189.122 | 1.00 | 18.16 |
| 3425 | CG | LEU | D | 340 | 47.443 | 97.047 | 190.119 | 1.00 | 25.80 |
| 3426 | CD1 | LEU | D | 340 | 47.122 | 97.518 | 191.509 | 1.00 | 65.83 |
| 3427 | CD2 | LEU | D | 340 | 46.525 | 95.915 | 189.742 | 1.00 | 77.47 |
| 3428 | C | LEU | D | 340 | 49.597 | 99.023 | 189.051 | 1.00 | 81.86 |
| 3429 | O | LEU | D | 340 | 49.935 | 98.238 | 188.174 | 1.00 | 80.36 |
| 3430 | N | SER | D | 341 | 50.456 | 99.560 | 189.912 | 1.00 | 80.46 |
| 3431 | CA | SER | D | 341 | 51.886 | 99.302 | 189.822 | 1.00 | 85.77 |
| 3432 | CB | SER | D | 341 | 52.656 | 100.405 | 190.549 | 1.00 | 101.32 |
| 3433 | OG | SER | D | 341 | 54.041 | 100.346 | 190.248 | 1.00 | 155.34 |
| 3434 | C | SER | D | 341 | 52.304 | 97.947 | 190.358 | 1.00 | 81.11 |
| 3435 | O | SER | D | 341 | 51.698 | 97.429 | 191.277 | 1.00 | 77.74 |
| 3436 | N | ARG | D | 342 | 53.355 | 97.388 | 189.766 | 1.00 | 106.18 |
| 3437 | CA | ARG | D | 342 | 53.912 | 96.091 | 190.150 | 1.00 | 44.48 |
| 3438 | CB | ARG | D | 342 | 54.380 | 95.361 | 188.892 | 1.00 | 103.52 |
| 3439 | CG | ARG | D | 342 | 55.115 | 96.289 | 187.906 | 1.00 | 146.45 |
| 3440 | CD | ARG | D | 342 | 55.845 | 95.557 | 186.765 | 1.00 | 148.55 |
| 3441 | NE | ARG | D | 342 | 54.964 | 95.031 | 185.722 | 1.00 | 106.07 |
| 3442 | CZ | ARG | D | 342 | 54.265 | 93.904 | 185.823 | 1.00 | 125.40 |
| 3443 | NH1 | ARG | D | 342 | 54.337 | 93.159 | 186.925 | 1.00 | 81.34 |
| 3444 | NH2 | ARG | D | 342 | 53.479 | 93.525 | 184.823 | 1.00 | 138.83 |
| 3445 | C | ARG | D | 342 | 55.100 | 96.277 | 191.117 | 1.00 | 93.34 |
| 3446 | O | ARG | D | 342 | 55.835 | 97.267 | 191.044 | 1.00 | 84.98 |
| 3447 | N | PRO | D | 343 | 55.309 | 95.312 | 192.025 | 1.00 | 51.92 |
| 3448 | CD | PRO | D | 343 | 54.613 | 94.020 | 192.055 | 1.00 | 46.08 |
| 3449 | CA | PRO | D | 343 | 56.390 | 95.336 | 193.017 | 1.00 | 37.46 |
| 3450 | CB | PRO | D | 343 | 56.323 | 93.946 | 193.638 | 1.00 | 54.05 |
| 3451 | CG | PRO | D | 343 | 54.925 | 93.534 | 193.435 | 1.00 | 34.48 |
| 3452 | C | PRO | D | 343 | 57.728 | 95.556 | 192.357 | 1.00 | 31.46 |
| 3453 | O | PRO | D | 343 | 58.023 | 94.920 | 191.352 | 1.00 | 83.41 |
| 3454 | N | SER | D | 344 | 58.555 | 96.430 | 192.906 | 1.00 | 68.87 |
| 3455 | CA | SER | D | 344 | 59.859 | 96.648 | 192.298 | 1.00 | 74.31 |
| 3456 | CB | SER | D | 344 | 60.476 | 97.969 | 192.781 | 1.00 | 78.99 |
| 3457 | OG | SER | D | 344 | 60.734 | 97.939 | 194.173 | 1.00 | 86.16 |
| 3458 | C | SER | D | 344 | 60.764 | 95.480 | 192.681 | 1.00 | 78.78 |
| 3459 | O | SER | D | 344 | 60.571 | 94.858 | 193.730 | 1.00 | 64.79 |
| 3460 | N | PRO | D | 345 | 61.744 | 95.149 | 191.822 | 1.00 | 78.82 |
| 3461 | CD | PRO | D | 345 | 62.114 | 95.844 | 190.579 | 1.00 | 86.56 |
| 3462 | CA | PRO | D | 345 | 62.673 | 94.054 | 192.096 | 1.00 | 69.52 |
| 3463 | CB | PRO | D | 345 | 63.792 | 94.311 | 191.101 | 1.00 | 81.00 |
| 3464 | CG | PRO | D | 345 | 63.078 | 94.883 | 189.947 | 1.00 | 56.65 |
| 3465 | C | PRO | D | 345 | 63.162 | 94.234 | 193.523 | 1.00 | 87.38 |
| 3466 | O | PRO | D | 345 | 63.009 | 93.356 | 194.382 | 1.00 | 70.47 |
| 3467 | N | PHE | D | 346 | 63.745 | 95.408 | 193.750 | 1.00 | 57.09 |
| 3468 | CA | PHE | D | 346 | 64.270 | 95.788 | 195.044 | 1.00 | 47.21 |
| 3469 | CB | PHE | D | 346 | 64.603 | 97.279 | 195.036 | 1.00 | 62.17 |
| 3470 | CG | PHE | D | 346 | 65.157 | 97.779 | 196.334 | 1.00 | 92.21 |
| 3471 | CD1 | PHE | D | 346 | 66.368 | 97.309 | 196.814 | 1.00 | 77.54 |
| 3472 | CD2 | PHE | D | 346 | 64.459 | 98.712 | 197.085 | 1.00 | 130.01 |
| 3473 | CE1 | PHE | D | 346 | 66.874 | 97.759 | 198.016 | 1.00 | 101.47 |
| 3474 | CE2 | PHE | D | 346 | 64.958 | 99.170 | 198.294 | 1.00 | 121.98 |
| 3475 | CZ | PHE | D | 346 | 66.167 | 98.693 | 198.761 | 1.00 | 136.63 |
| 3476 | C | PHE | D | 346 | 63.300 | 95.463 | 196.180 | 1.00 | 61.69 |
| 3477 | O | PHE | D | 346 | 63.557 | 94.562 | 196.976 | 1.00 | 58.05 |
| 3478 | N | ASP | D | 347 | 62.186 | 96.182 | 196.250 | 1.00 | 49.53 |
| 3479 | CA | ASP | D | 347 | 61.199 | 95.958 | 197.302 | 1.00 | 60.77 |
| 3480 | CB | ASP | D | 347 | 59.939 | 96.792 | 197.044 | 1.00 | 97.33 |
| 3481 | CG | ASP | D | 347 | 60.121 | 98.260 | 197.380 | 1.00 | 120.23 |
| 3482 | OD1 | ASP | D | 347 | 60.197 | 98.590 | 198.583 | 1.00 | 142.30 |
| 3483 | OD2 | ASP | D | 347 | 60.189 | 99.084 | 196.443 | 1.00 | 147.88 |
| 3484 | C | ASP | D | 347 | 60.791 | 94.494 | 197.407 | 1.00 | 79.99 |
| 3485 | O | ASP | D | 347 | 60.144 | 94.086 | 198.373 | 1.00 | 51.27 |
| 3486 | N | LEU | D | 348 | 61.191 | 93.697 | 196.427 | 1.00 | 54.00 |
| 3487 | CA | LEU | D | 348 | 60.791 | 92.304 | 196.400 | 1.00 | 73.58 |
| 3488 | CB | LEU | D | 348 | 60.293 | 91.977 | 194.991 | 1.00 | 43.65 |
| 3489 | CG | LEU | D | 348 | 59.764 | 90.586 | 194.663 | 1.00 | 61.62 |
| 3490 | CD1 | LEU | D | 348 | 58.851 | 90.063 | 195.737 | 1.00 | 73.74 |
| 3491 | CD2 | LEU | D | 348 | 59.041 | 90.673 | 193.342 | 1.00 | 84.11 |
| 3492 | C | LEU | D | 348 | 61.823 | 91.287 | 196.838 | 1.00 | 79.18 |
| 3493 | O | LEU | D | 348 | 61.456 | 90.240 | 197.381 | 1.00 | 67.98 |
| 3494 | N | PHE | D | 349 | 63.103 | 91.596 | 196.617 | 1.00 | 89.01 |
| 3495 | CA | PHE | D | 349 | 64.195 | 90.681 | 196.959 | 1.00 | 97.99 |
| 3496 | CB | PHE | D | 349 | 64.993 | 90.401 | 195.706 | 1.00 | 91.42 |
| 3497 | CG | PHE | D | 349 | 64.191 | 89.710 | 194.656 | 1.00 | 89.32 |
| 3498 | CD1 | PHE | D | 349 | 64.403 | 89.967 | 193.315 | 1.00 | 93.99 |
| 3499 | CD2 | PHE | D | 349 | 63.194 | 88.804 | 195.021 | 1.00 | 58.86 |
| 3500 | CE1 | PHE | D | 349 | 63.633 | 89.339 | 192.352 | 1.00 | 60.78 |
| 3501 | CE2 | PHE | D | 349 | 62.420 | 88.171 | 194.061 | 1.00 | 91.28 |
| 3502 | CZ | PHE | D | 349 | 62.639 | 88.439 | 192.724 | 1.00 | 94.91 |
| 3503 | C | PHE | D | 349 | 65.098 | 91.150 | 198.076 | 1.00 | 102.56 |
| 3504 | O | PHE | D | 349 | 65.470 | 90.369 | 198.955 | 1.00 | 116.27 |
| 3505 | N | ILE | D | 350 | 65.464 | 92.422 | 198.033 | 1.00 | 96.29 |
| 3506 | CA | ILE | D | 350 | 66.276 | 92.993 | 199.088 | 1.00 | 73.68 |
| 3507 | CB | ILE | D | 350 | 66.804 | 94.381 | 198.700 | 1.00 | 73.26 |
| 3508 | CG2 | ILE | D | 350 | 67.594 | 94.970 | 199.850 | 1.00 | 90.70 |
| 3509 | CG1 | ILE | D | 350 | 67.638 | 94.282 | 197.428 | 1.00 | 62.89 |
| 3510 | CD1 | ILE | D | 350 | 68.682 | 93.207 | 197.474 | 1.00 | 77.68 |
| 3511 | C | ILE | D | 350 | 65.337 | 93.162 | 200.286 | 1.00 | 91.79 |
| 3512 | O | ILE | D | 350 | 65.229 | 92.277 | 201.143 | 1.00 | 96.73 |
| 3513 | N | ARG | D | 351 | 64.639 | 94.299 | 200.302 | 1.00 | 79.05 |
| 3514 | CA | ARG | D | 351 | 63.696 | 94.661 | 201.356 | 1.00 | 77.91 |
| 3515 | CB | ARG | D | 351 | 62.908 | 95.894 | 200.909 | 1.00 | 81.92 |
| 3516 | CG | ARG | D | 351 | 62.429 | 96.796 | 202.033 | 1.00 | 114.59 |
| 3517 | CD | ARG | D | 351 | 62.218 | 98.216 | 201.513 | 1.00 | 129.04 |
| 3518 | NE | ARG | D | 351 | 61.772 | 99.135 | 202.556 | 1.00 | 176.58 |
| 3519 | CZ | ARG | D | 351 | 60.594 | 99.057 | 203.165 | 1.00 | 194.68 |
| 3520 | NH1 | ARG | D | 351 | 59.738 | 98.100 | 202.835 | 1.00 | 199.64 |
| 3521 | NH2 | ARG | D | 351 | 60.271 | 99.937 | 204.105 | 1.00 | 191.83 |
| 3522 | C | ARG | D | 351 | 62.738 | 93.529 | 201.737 | 1.00 | 87.28 |
| 3523 | O | ARG | D | 351 | 62.129 | 93.558 | 202.807 | 1.00 | 85.89 |
| 3524 | N | LYS | D | 352 | 62.620 | 92.536 | 200.860 | 1.00 | 64.64 |
| 3525 | CA | LYS | D | 352 | 61.754 | 91.375 | 201.066 | 1.00 | 110.09 |
| 3526 | CB | LYS | D | 352 | 62.443 | 90.351 | 201.977 | 1.00 | 137.68 |
| 3527 | CG | LYS | D | 352 | 63.695 | 89.710 | 201.380 | 1.00 | 164.74 |
| 3528 | CD | LYS | D | 352 | 64.045 | 88.410 | 202.099 | 1.00 | 184.77 |
| 3529 | CE | LYS | D | 352 | 65.381 | 87.834 | 201.642 | 1.00 | 171.68 |
| 3530 | NZ | LYS | D | 352 | 66.539 | 88.623 | 202.153 | 1.00 | 185.42 |
| 3531 | C | LYS | D | 352 | 60.339 | 91.659 | 201.587 | 1.00 | 121.79 |
| 3532 | O | LYS | D | 352 | 59.714 | 90.793 | 202.198 | 1.00 | 115.18 |
| 3533 | N | SER | D | 353 | 59.841 | 92.869 | 201.344 | 1.00 | 129.12 |
| 3534 | CA | SER | D | 353 | 58.484 | 93.265 | 201.740 | 1.00 | 107.18 |
| 3535 | CB | SER | D | 353 | 58.506 | 94.174 | 202.975 | 1.00 | 122.87 |
| 3536 | OG | SER | D | 353 | 59.158 | 95.404 | 202.707 | 1.00 | 166.93 |
| 3537 | C | SER | D | 353 | 57.907 | 94.012 | 200.534 | 1.00 | 102.01 |
| 3538 | O | SER | D | 353 | 57.957 | 95.243 | 200.446 | 1.00 | 86.01 |
| 3539 | N | PRO | D | 354 | 57.350 | 93.261 | 199.578 | 1.00 | 86.76 |
| 3540 | CD | PRO | D | 354 | 57.251 | 91.797 | 199.555 | 1.00 | 96.08 |
| 3541 | CA | PRO | D | 354 | 56.770 | 93.816 | 198.361 | 1.00 | 78.36 |
| 3542 | CB | PRO | D | 354 | 56.785 | 92.630 | 197.393 | 1.00 | 84.14 |
| 3543 | CG | PRO | D | 354 | 57.526 | 91.526 | 198.127 | 1.00 | 77.25 |
| 3544 | C | PRO | D | 354 | 55.367 | 94.332 | 198.547 | 1.00 | 94.58 |
| 3545 | O | PRO | D | 354 | 54.605 | 93.797 | 199.350 | 1.00 | 69.20 |
| 3546 | N | THR | D | 355 | 55.033 | 95.366 | 197.781 | 1.00 | 81.15 |
| 3547 | CA | THR | D | 355 | 53.706 | 95.957 | 197.813 | 1.00 | 52.66 |
| 3548 | CB | THR | D | 355 | 53.598 | 97.082 | 198.827 | 1.00 | 55.40 |
| 3549 | OG1 | THR | D | 355 | 54.557 | 98.101 | 198.513 | 1.00 | 77.41 |
| 3550 | CG2 | THR | D | 355 | 53.816 | 96.551 | 200.232 | 1.00 | 118.83 |
| 3551 | C | THR | D | 355 | 53.328 | 96.554 | 196.475 | 1.00 | 73.36 |
| 3552 | O | THR | D | 355 | 54.123 | 97.269 | 195.852 | 1.00 | 60.58 |
| 3553 | N | ILE | D | 356 | 52.100 | 96.261 | 196.054 | 1.00 | 68.63 |
| 3554 | CA | ILE | D | 356 | 51.550 | 96.771 | 194.809 | 1.00 | 71.72 |
| 3555 | CB | ILE | D | 356 | 50.725 | 95.712 | 194.105 | 1.00 | 33.12 |
| 3556 | CG2 | ILE | D | 356 | 51.621 | 94.618 | 193.613 | 1.00 | 84.96 |
| 3557 | CG1 | ILE | D | 356 | 49.693 | 95.138 | 195.059 | 1.00 | 69.76 |
| 3558 | CD1 | ILE | D | 356 | 48.895 | 93.992 | 194.466 | 1.00 | 78.12 |
| 3559 | C | ILE | D | 356 | 50.660 | 97.956 | 195.140 | 1.00 | 56.65 |
| 3560 | O | ILE | D | 356 | 50.092 | 98.020 | 196.214 | 1.00 | 40.32 |
| 3561 | N | THR | D | 357 | 50.527 | 98.886 | 194.210 | 1.00 | 41.53 |
| 3562 | CA | THR | D | 357 | 49.738 | 100.075 | 194.453 | 1.00 | 56.47 |
| 3563 | CB | THR | D | 357 | 50.661 | 101.268 | 194.609 | 1.00 | 44.57 |
| 3564 | OG1 | THR | D | 357 | 51.732 | 100.908 | 195.484 | 1.00 | 86.28 |
| 3565 | CG2 | THR | D | 357 | 49.919 | 102.440 | 195.180 | 1.00 | 70.27 |
| 3566 | C | THR | D | 357 | 48.719 | 100.390 | 193.367 | 1.00 | 61.21 |
| 3567 | O | THR | D | 357 | 49.043 | 100.408 | 192.182 | 1.00 | 65.37 |
| 3568 | N | CYS | D | 358 | 47.487 | 100.656 | 193.794 | 1.00 | 57.66 |
| 3569 | CA | CYS | D | 358 | 46.388 | 100.989 | 192.898 | 1.00 | 62.45 |
| 3570 | C | CYS | D | 358 | 46.155 | 102.478 | 192.983 | 1.00 | 45.63 |
| 3571 | O | CYS | D | 358 | 45.867 | 102.993 | 194.054 | 1.00 | 74.78 |
| 3572 | CB | CYS | D | 358 | 45.135 | 100.263 | 193.341 | 1.00 | 61.30 |
| 3573 | SG | CYS | D | 358 | 43.775 | 100.299 | 192.140 | 1.00 | 68.08 |
| 3574 | N | LEU | D | 359 | 46.272 | 103.174 | 191.864 | 1.00 | 41.70 |
| 3575 | CA | LEU | D | 359 | 46.105 | 104.616 | 191.868 | 1.00 | 25.08 |
| 3576 | CB | LEU | D | 359 | 47.404 | 105.279 | 191.410 | 1.00 | 65.10 |
| 3577 | CG | LEU | D | 359 | 47.322 | 106.716 | 190.905 | 1.00 | 16.89 |
| 3578 | CD1 | LEU | D | 359 | 46.716 | 107.566 | 191.963 | 1.00 | 57.67 |
| 3579 | CD2 | LEU | D | 359 | 48.703 | 107.229 | 190.549 | 1.00 | 96.64 |
| 3580 | C | LEU | D | 359 | 44.954 | 105.063 | 190.986 | 1.00 | 44.20 |
| 3581 | O | LEU | D | 359 | 44.956 | 104.830 | 189.784 | 1.00 | 68.36 |
| 3582 | N | VAL | D | 360 | 43.971 | 105.715 | 191.591 | 1.00 | 50.01 |
| 3583 | CA | VAL | D | 360 | 42.821 | 106.193 | 190.853 | 1.00 | 29.75 |
| 3584 | CB | VAL | D | 360 | 41.539 | 105.909 | 191.602 | 1.00 | 34.70 |
| 3585 | CG1 | VAL | D | 360 | 40.388 | 106.459 | 190.821 | 1.00 | 38.83 |
| 3586 | CG2 | VAL | D | 360 | 41.365 | 104.422 | 191.810 | 1.00 | 28.01 |
| 3587 | C | VAL | D | 360 | 42.927 | 107.681 | 190.661 | 1.00 | 28.40 |
| 3588 | O | VAL | D | 360 | 43.209 | 108.404 | 191.601 | 1.00 | 60.69 |
| 3589 | N | VAL | D | 361 | 42.677 | 108.146 | 189.450 | 1.00 | 28.27 |
| 3590 | CA | VAL | D | 361 | 42.771 | 109.574 | 189.156 | 1.00 | 32.16 |
| 3591 | CB | VAL | D | 361 | 44.007 | 109.853 | 188.272 | 1.00 | 10.08 |
| 3592 | CG1 | VAL | D | 361 | 44.974 | 108.690 | 188.387 | 1.00 | 49.51 |
| 3593 | CG2 | VAL | D | 361 | 43.595 | 110.013 | 186.820 | 1.00 | 48.77 |
| 3594 | C | VAL | D | 361 | 41.525 | 110.131 | 188.458 | 1.00 | 26.83 |
| 3595 | O | VAL | D | 361 | 40.695 | 109.389 | 187.937 | 1.00 | 48.72 |
| 3596 | N | ASP | D | 362 | 41.422 | 111.452 | 188.433 | 1.00 | 44.02 |
| 3597 | CA | ASP | D | 362 | 40.308 | 112.142 | 187.804 | 1.00 | 31.26 |
| 3598 | CB | ASP | D | 362 | 40.227 | 111.794 | 186.324 | 1.00 | 57.54 |
| 3599 | CG | ASP | D | 362 | 41.377 | 112.380 | 185.527 | 1.00 | 99.45 |
| 3600 | OD1 | ASP | D | 362 | 41.751 | 113.542 | 185.804 | 1.00 | 59.82 |
| 3601 | OD2 | ASP | D | 362 | 41.893 | 111.684 | 184.620 | 1.00 | 97.18 |
| 3602 | C | ASP | D | 362 | 38.945 | 111.946 | 188.458 | 1.00 | 45.28 |
| 3603 | O | ASP | D | 362 | 37.914 | 112.146 | 187.816 | 1.00 | 58.17 |
| 3604 | N | LEU | D | 363 | 38.943 | 111.554 | 189.730 | 1.00 | 31.65 |
| 3605 | CA | LEU | D | 363 | 37.709 | 111.405 | 190.496 | 1.00 | 30.37 |
| 3606 | CB | LEU | D | 363 | 37.964 | 110.712 | 191.820 | 1.00 | 14.90 |
| 3607 | CG | LEU | D | 363 | 38.139 | 109.208 | 191.819 | 1.00 | 39.19 |
| 3608 | CD1 | LEU | D | 363 | 38.611 | 108.750 | 193.177 | 1.00 | 47.04 |
| 3609 | CD2 | LEU | D | 363 | 36.839 | 108.553 | 191.480 | 1.00 | 35.75 |
| 3610 | C | LEU | D | 363 | 37.243 | 112.814 | 190.820 | 1.00 | 68.38 |
| 3611 | O | LEU | D | 363 | 38.045 | 113.751 | 190.805 | 1.00 | 53.90 |
| 3612 | N | ALA | D | 364 | 35.962 | 112.974 | 191.142 | 1.00 | 116.09 |
| 3613 | CA | ALA | D | 364 | 35.443 | 114.297 | 191.475 | 1.00 | 94.58 |
| 3614 | CB | ALA | D | 364 | 34.731 | 114.906 | 190.280 | 1.00 | 92.82 |
| 3615 | C | ALA | D | 364 | 34.498 | 114.228 | 192.656 | 1.00 | 118.54 |
| 3616 | O | ALA | D | 364 | 33.729 | 113.273 | 192.797 | 1.00 | 129.25 |
| 3617 | N | PRO | D | 365 | 34.548 | 115.252 | 193.524 | 1.00 | 128.97 |
| 3618 | CD | PRO | D | 365 | 35.391 | 116.455 | 193.354 | 1.00 | 57.81 |
| 3619 | CA | PRO | D | 365 | 33.710 | 115.360 | 194.723 | 1.00 | 105.98 |
| 3620 | CB | PRO | D | 365 | 33.666 | 116.860 | 194.958 | 1.00 | 121.84 |
| 3621 | CG | PRO | D | 365 | 35.087 | 117.254 | 194.616 | 1.00 | 78.18 |
| 3622 | C | PRO | D | 365 | 32.323 | 114.734 | 194.549 | 1.00 | 91.22 |
| 3623 | O | PRO | D | 365 | 31.436 | 115.298 | 193.916 | 1.00 | 85.50 |
| 3624 | N | SER | D | 366 | 32.162 | 113.542 | 195.107 | 1.00 | 132.49 |
| 3625 | CA | SER | D | 366 | 30.904 | 112.816 | 195.033 | 1.00 | 136.44 |
| 3626 | CB | SER | D | 366 | 31.149 | 111.400 | 194.503 | 1.00 | 121.82 |
| 3627 | OG | SER | D | 366 | 32.105 | 110.710 | 195.302 | 1.00 | 115.17 |
| 3628 | C | SER | D | 366 | 30.313 | 112.752 | 196.438 | 1.00 | 176.44 |
| 3629 | O | SER | D | 366 | 31.031 | 112.918 | 197.428 | 1.00 | 164.25 |
| 3630 | N | LYS | D | 367 | 29.007 | 112.523 | 196.524 | 1.00 | 189.75 |
| 3631 | CA | LYS | D | 367 | 28.341 | 112.430 | 197.819 | 1.00 | 181.41 |
| 3632 | CB | LYS | D | 367 | 26.896 | 112.910 | 197.720 | 1.00 | 196.81 |
| 3633 | CG | LYS | D | 367 | 26.739 | 114.334 | 197.233 | 1.00 | 225.05 |
| 3634 | CD | LYS | D | 367 | 27.059 | 115.373 | 198.291 | 1.00 | 212.98 |
| 3635 | CE | LYS | D | 367 | 26.738 | 116.763 | 197.760 | 1.00 | 208.48 |
| 3636 | NZ | LYS | D | 367 | 26.913 | 117.823 | 198.784 | 1.00 | 202.36 |
| 3637 | C | LYS | D | 367 | 28.355 | 110.982 | 198.274 | 1.00 | 162.45 |
| 3638 | O | LYS | D | 367 | 28.021 | 110.677 | 199.416 | 1.00 | 165.32 |
| 3639 | N | GLY | D | 368 | 28.735 | 110.093 | 197.365 | 1.00 | 160.80 |
| 3640 | CA | GLY | D | 368 | 28.791 | 108.685 | 197.696 | 1.00 | 152.04 |
| 3641 | C | GLY | D | 368 | 30.217 | 108.220 | 197.912 | 1.00 | 174.77 |
| 3642 | O | GLY | D | 368 | 31.174 | 108.928 | 197.582 | 1.00 | 170.78 |
| 3643 | N | THR | D | 369 | 30.355 | 107.023 | 198.476 | 1.00 | 173.28 |
| 3644 | CA | THR | D | 369 | 31.661 | 106.424 | 198.740 | 1.00 | 158.08 |
| 3645 | CB | THR | D | 369 | 31.492 | 105.030 | 199.398 | 1.00 | 168.98 |
| 3646 | OG1 | THR | D | 369 | 30.447 | 105.089 | 200.378 | 1.00 | 140.65 |
| 3647 | CG2 | THR | D | 369 | 32.788 | 104.590 | 200.079 | 1.00 | 155.41 |
| 3648 | C | THR | D | 369 | 32.375 | 106.253 | 197.396 | 1.00 | 126.97 |
| 3649 | O | THR | D | 369 | 31.908 | 106.754 | 196.380 | 1.00 | 121.57 |
| 3650 | N | VAL | D | 370 | 33.514 | 105.566 | 197.397 | 1.00 | 124.90 |
| 3651 | CA | VAL | D | 370 | 34.254 | 105.306 | 196.166 | 1.00 | 58.92 |
| 3652 | CB | VAL | D | 370 | 35.273 | 106.414 | 195.851 | 1.00 | 55.35 |
| 3653 | CG1 | VAL | D | 370 | 35.805 | 106.246 | 194.401 | 1.00 | 24.89 |
| 3654 | CG2 | VAL | D | 370 | 34.616 | 107.793 | 196.044 | 1.00 | 62.46 |
| 3655 | C | VAL | D | 370 | 34.955 | 103.979 | 196.320 | 1.00 | 27.33 |
| 3656 | O | VAL | D | 370 | 36.068 | 103.773 | 195.862 | 1.00 | 70.43 |
| 3657 | N | ASN | D | 371 | 34.256 | 103.075 | 196.988 | 1.00 | 82.80 |
| 3658 | CA | ASN | D | 371 | 34.734 | 101.724 | 197.243 | 1.00 | 69.31 |
| 3659 | CB | ASN | D | 371 | 33.595 | 100.719 | 196.996 | 1.00 | 49.52 |
| 3660 | CG | ASN | D | 371 | 32.950 | 100.229 | 198.274 | 1.00 | 104.67 |
| 3661 | OD1 | ASN | D | 371 | 32.407 | 101.025 | 199.043 | 1.00 | 117.80 |
| 3662 | ND2 | ASN | D | 371 | 32.979 | 98.919 | 198.497 | 1.00 | 168.88 |
| 3663 | C | ASN | D | 371 | 35.958 | 101.252 | 196.469 | 1.00 | 80.11 |
| 3664 | O | ASN | D | 371 | 35.988 | 101.271 | 195.234 | 1.00 | 50.10 |
| 3665 | N | LEU | D | 372 | 36.972 | 100.847 | 197.218 | 1.00 | 87.75 |
| 3666 | CA | LEU | D | 372 | 38.168 | 100.270 | 196.635 | 1.00 | 40.25 |
| 3667 | CB | LEU | D | 372 | 39.399 | 101.036 | 197.078 | 1.00 | 51.63 |
| 3668 | CG | LEU | D | 372 | 40.698 | 100.819 | 196.321 | 1.00 | 42.05 |
| 3669 | CD1 | LEU | D | 372 | 40.673 | 99.475 | 195.610 | 1.00 | 57.36 |
| 3670 | CD2 | LEU | D | 372 | 40.854 | 101.961 | 195.344 | 1.00 | 17.22 |
| 3671 | C | LEU | D | 372 | 38.164 | 98.893 | 197.302 | 1.00 | 69.49 |
| 3672 | O | LEU | D | 372 | 37.925 | 98.774 | 198.504 | 1.00 | 99.46 |
| 3673 | N | THR | D | 373 | 38.398 | 97.852 | 196.524 | 1.00 | 50.31 |
| 3674 | CA | THR | D | 373 | 38.367 | 96.516 | 197.078 | 1.00 | 38.63 |
| 3675 | CB | THR | D | 373 | 36.958 | 95.894 | 196.859 | 1.00 | 63.65 |
| 3676 | OG1 | THR | D | 373 | 35.980 | 96.704 | 197.523 | 1.00 | 71.89 |
| 3677 | CG2 | THR | D | 373 | 36.905 | 94.478 | 197.408 | 1.00 | 89.69 |
| 3678 | C | THR | D | 373 | 39.435 | 95.613 | 196.478 | 1.00 | 74.34 |
| 3679 | O | THR | D | 373 | 39.493 | 95.440 | 195.262 | 1.00 | 67.92 |
| 3680 | N | TRP | D | 374 | 40.273 | 95.032 | 197.335 | 1.00 | 71.85 |
| 3681 | CA | TRP | D | 374 | 41.343 | 94.148 | 196.885 | 1.00 | 42.08 |
| 3682 | CB | TRP | D | 374 | 42.566 | 94.309 | 197.769 | 1.00 | 57.45 |
| 3683 | CG | TRP | D | 374 | 43.280 | 95.595 | 197.614 | 1.00 | 41.03 |
| 3684 | CD2 | TRP | D | 374 | 44.219 | 95.924 | 196.593 | 1.00 | 30.13 |
| 3685 | CE2 | TRP | D | 374 | 44.648 | 97.244 | 196.827 | 1.00 | 32.56 |
| 3686 | CE3 | TRP | D | 374 | 44.740 | 95.229 | 195.500 | 1.00 | 52.50 |
| 3687 | CD1 | TRP | D | 374 | 43.176 | 96.697 | 198.410 | 1.00 | 58.12 |
| 3688 | NE1 | TRP | D | 374 | 44.000 | 97.697 | 197.944 | 1.00 | 31.82 |
| 3689 | CZ2 | TRP | D | 374 | 45.570 | 97.881 | 196.006 | 1.00 | 53.35 |
| 3690 | CZ3 | TRP | D | 374 | 45.658 | 95.864 | 194.684 | 1.00 | 47.95 |
| 3691 | CH2 | TRP | D | 374 | 46.064 | 97.175 | 194.939 | 1.00 | 23.35 |
| 3692 | C | TRP | D | 374 | 40.954 | 92.681 | 196.910 | 1.00 | 85.67 |
| 3693 | O | TRP | D | 374 | 40.261 | 92.236 | 197.822 | 1.00 | 80.11 |
| 3694 | N | SER | D | 375 | 41.434 | 91.916 | 195.936 | 1.00 | 64.91 |
| 3695 | CA | SER | D | 375 | 41.110 | 90.496 | 195.878 | 1.00 | 80.39 |
| 3696 | CB | SER | D | 375 | 39.737 | 90.325 | 195.236 | 1.00 | 121.93 |
| 3697 | OG | SER | D | 375 | 39.703 | 90.935 | 193.956 | 1.00 | 111.06 |
| 3698 | C | SER | D | 375 | 42.135 | 89.659 | 195.114 | 1.00 | 105.31 |
| 3699 | O | SER | D | 375 | 42.802 | 90.157 | 194.202 | 1.00 | 54.37 |
| 3700 | N | ARG | D | 376 | 42.253 | 88.386 | 195.493 | 1.00 | 87.71 |
| 3701 | CA | ARG | D | 376 | 43.174 | 87.466 | 194.831 | 1.00 | 90.92 |
| 3702 | CB | ARG | D | 376 | 43.974 | 86.665 | 195.854 | 1.00 | 101.35 |
| 3703 | CG | ARG | D | 376 | 45.158 | 87.402 | 196.423 | 1.00 | 114.89 |
| 3704 | CD | ARG | D | 376 | 46.249 | 86.437 | 196.863 | 1.00 | 123.05 |
| 3705 | NE | ARG | D | 376 | 46.021 | 85.871 | 198.187 | 1.00 | 92.73 |
| 3706 | CZ | ARG | D | 376 | 46.833 | 84.992 | 198.763 | 1.00 | 124.05 |
| 3707 | NH1 | ARG | D | 376 | 47.920 | 84.580 | 198.128 | 1.00 | 110.55 |
| 3708 | NH2 | ARG | D | 376 | 46.567 | 84.534 | 199.978 | 1.00 | 162.37 |
| 3709 | C | ARG | D | 376 | 42.420 | 86.499 | 193.927 | 1.00 | 117.75 |
| 3710 | O | ARG | D | 376 | 41.260 | 86.168 | 194.186 | 1.00 | 105.90 |
| 3711 | N | ALA | D | 377 | 43.082 | 86.039 | 192.871 | 1.00 | 99.06 |
| 3712 | CA | ALA | D | 377 | 42.453 | 85.109 | 191.944 | 1.00 | 110.56 |
| 3713 | CB | ALA | D | 377 | 43.235 | 85.059 | 190.659 | 1.00 | 96.70 |
| 3714 | C | ALA | D | 377 | 42.355 | 83.715 | 192.550 | 1.00 | 113.96 |
| 3715 | O | ALA | D | 377 | 41.454 | 82.940 | 192.218 | 1.00 | 121.96 |
| 3716 | N | SER | D | 378 | 43.288 | 83.400 | 193.441 | 1.00 | 100.05 |
| 3717 | CA | SER | D | 378 | 43.299 | 82.102 | 194.096 | 1.00 | 74.96 |
| 3718 | CB | SER | D | 378 | 44.607 | 81.910 | 194.863 | 1.00 | 108.91 |
| 3719 | OG | SER | D | 378 | 44.639 | 82.730 | 196.015 | 1.00 | 102.13 |
| 3720 | C | SER | D | 378 | 42.119 | 81.973 | 195.060 | 1.00 | 87.98 |
| 3721 | O | SER | D | 378 | 41.716 | 80.868 | 195.415 | 1.00 | 117.47 |
| 3722 | N | GLY | D | 379 | 41.569 | 83.105 | 195.478 | 1.00 | 88.22 |
| 3723 | CA | GLY | D | 379 | 40.452 | 83.080 | 196.403 | 1.00 | 101.70 |
| 3724 | C | GLY | D | 379 | 40.922 | 83.261 | 197.835 | 1.00 | 122.17 |
| 3725 | O | GLY | D | 379 | 40.135 | 83.578 | 198.733 | 1.00 | 104.23 |
| 3726 | N | LYS | D | 380 | 42.218 | 83.064 | 198.047 | 1.00 | 123.72 |
| 3727 | CA | LYS | D | 380 | 42.805 | 83.202 | 199.373 | 1.00 | 148.58 |
| 3728 | CB | LYS | D | 380 | 44.260 | 82.727 | 199.348 | 1.00 | 164.15 |
| 3729 | CG | LYS | D | 380 | 44.446 | 81.293 | 198.871 | 1.00 | 172.83 |
| 3730 | CD | LYS | D | 380 | 45.922 | 80.912 | 198.838 | 1.00 | 182.01 |
| 3731 | CE | LYS | D | 380 | 46.121 | 79.480 | 198.365 | 1.00 | 174.42 |
| 3732 | NZ | LYS | D | 380 | 47.562 | 79.099 | 198.345 | 1.00 | 158.73 |
| 3733 | C | LYS | D | 380 | 42.737 | 84.654 | 199.854 | 1.00 | 146.29 |
| 3734 | O | LYS | D | 380 | 43.016 | 85.582 | 199.096 | 1.00 | 155.06 |
| 3735 | N | PRO | D | 381 | 42.360 | 84.863 | 201.126 | 1.00 | 140.56 |
| 3736 | CD | PRO | D | 381 | 41.968 | 83.800 | 202.069 | 1.00 | 154.32 |
| 3737 | CA | PRO | D | 381 | 42.240 | 86.184 | 201.757 | 1.00 | 132.74 |
| 3738 | CB | PRO | D | 381 | 42.074 | 85.834 | 203.231 | 1.00 | 144.55 |
| 3739 | CG | PRO | D | 381 | 41.264 | 84.578 | 203.163 | 1.00 | 138.88 |
| 3740 | C | PRO | D | 381 | 43.428 | 87.119 | 201.507 | 1.00 | 118.02 |
| 3741 | O | PRO | D | 381 | 44.490 | 86.675 | 201.074 | 1.00 | 99.94 |
| 3742 | N | VAL | D | 382 | 43.239 | 88.410 | 201.793 | 1.00 | 105.18 |
| 3743 | CA | VAL | D | 382 | 44.279 | 89.429 | 201.584 | 1.00 | 80.19 |
| 3744 | CB | VAL | D | 382 | 43.901 | 90.375 | 200.458 | 1.00 | 64.06 |
| 3745 | CG1 | VAL | D | 382 | 43.890 | 89.629 | 199.140 | 1.00 | 134.59 |
| 3746 | CG2 | VAL | D | 382 | 42.530 | 90.963 | 200.739 | 1.00 | 87.60 |
| 3747 | C | VAL | D | 382 | 44.553 | 90.305 | 202.791 | 1.00 | 102.12 |
| 3748 | O | VAL | D | 382 | 43.654 | 90.583 | 203.581 | 1.00 | 116.29 |
| 3749 | N | ASN | D | 383 | 45.796 | 90.764 | 202.913 | 1.00 | 100.94 |
| 3750 | CA | ASN | D | 383 | 46.189 | 91.618 | 204.033 | 1.00 | 127.59 |
| 3751 | CB | ASN | D | 383 | 47.694 | 91.910 | 203.982 | 1.00 | 137.48 |
| 3752 | CG | ASN | D | 383 | 48.543 | 90.699 | 204.346 | 1.00 | 150.77 |
| 3753 | OD1 | ASN | D | 383 | 49.767 | 90.707 | 204.178 | 1.00 | 132.07 |
| 3754 | ND2 | ASN | D | 383 | 47.897 | 89.654 | 204.857 | 1.00 | 146.01 |
| 3755 | C | ASN | D | 383 | 45.411 | 92.928 | 203.998 | 1.00 | 88.67 |
| 3756 | O | ASN | D | 383 | 44.731 | 93.223 | 203.021 | 1.00 | 96.54 |
| 3757 | N | HIS | D | 384 | 45.510 | 93.708 | 205.068 | 1.00 | 90.17 |
| 3758 | CA | HIS | D | 384 | 44.810 | 94.985 | 205.142 | 1.00 | 76.61 |
| 3759 | CB | HIS | D | 384 | 44.747 | 95.464 | 206.594 | 1.00 | 88.84 |
| 3760 | CG | HIS | D | 384 | 43.971 | 94.546 | 207.492 | 1.00 | 115.42 |
| 3761 | CD2 | HIS | D | 384 | 44.367 | 93.491 | 208.244 | 1.00 | 119.90 |
| 3762 | ND1 | HIS | D | 384 | 42.602 | 94.630 | 207.639 | 1.00 | 93.91 |
| 3763 | CE1 | HIS | D | 384 | 42.189 | 93.665 | 208.443 | 1.00 | 115.01 |
| 3764 | NE2 | HIS | D | 384 | 43.239 | 92.960 | 208.823 | 1.00 | 139.25 |
| 3765 | C | HIS | D | 384 | 45.538 | 95.996 | 204.281 | 1.00 | 71.38 |
| 3766 | O | HIS | D | 384 | 46.757 | 96.000 | 204.219 | 1.00 | 48.03 |
| 3767 | N | SER | D | 385 | 44.787 | 96.859 | 203.617 | 1.00 | 81.66 |
| 3768 | CA | SER | D | 385 | 45.396 | 97.845 | 202.748 | 1.00 | 36.20 |
| 3769 | CB | SER | D | 385 | 44.638 | 97.893 | 201.424 | 1.00 | 66.21 |
| 3770 | OG | SER | D | 385 | 43.265 | 98.161 | 201.638 | 1.00 | 99.18 |
| 3771 | C | SER | D | 385 | 45.467 | 99.247 | 203.338 | 1.00 | 61.33 |
| 3772 | O | SER | D | 385 | 44.928 | 99.529 | 204.404 | 1.00 | 72.76 |
| 3773 | N | THR | D | 386 | 46.130 | 100.119 | 202.601 | 1.00 | 38.22 |
| 3774 | CA | THR | D | 386 | 46.341 | 101.507 | 202.949 | 1.00 | 30.42 |
| 3775 | CB | THR | D | 386 | 47.810 | 101.868 | 202.631 | 1.00 | 78.59 |
| 3776 | OG1 | THR | D | 386 | 48.625 | 101.545 | 203.761 | 1.00 | 109.39 |
| 3777 | CG2 | THR | D | 386 | 47.973 | 103.333 | 202.247 | 1.00 | 82.78 |
| 3778 | C | THR | D | 386 | 45.416 | 102.332 | 202.086 | 1.00 | 55.16 |
| 3779 | O | THR | D | 386 | 44.858 | 101.819 | 201.124 | 1.00 | 85.45 |
| 3780 | N | ARG | D | 387 | 45.240 | 103.608 | 202.412 | 1.00 | 60.12 |
| 3781 | CA | ARG | D | 387 | 44.399 | 104.459 | 201.569 | 1.00 | 57.26 |
| 3782 | CB | ARG | D | 387 | 42.956 | 104.032 | 201.709 | 1.00 | 45.25 |
| 3783 | CG | ARG | D | 387 | 41.990 | 104.919 | 200.996 | 1.00 | 58.55 |
| 3784 | CD | ARG | D | 387 | 40.661 | 104.219 | 200.838 | 1.00 | 62.37 |
| 3785 | NE | ARG | D | 387 | 39.669 | 105.097 | 200.233 | 1.00 | 75.16 |
| 3786 | CZ | ARG | D | 387 | 38.472 | 104.694 | 199.827 | 1.00 | 113.22 |
| 3787 | NH1 | ARG | D | 387 | 38.125 | 103.418 | 199.965 | 1.00 | 105.83 |
| 3788 | NH2 | ARG | D | 387 | 37.627 | 105.564 | 199.282 | 1.00 | 88.31 |
| 3789 | C | ARG | D | 387 | 44.528 | 105.952 | 201.834 | 1.00 | 49.54 |
| 3790 | O | ARG | D | 387 | 44.421 | 106.373 | 202.977 | 1.00 | 74.17 |
| 3791 | N | LYS | D | 388 | 44.759 | 106.743 | 200.784 | 1.00 | 14.61 |
| 3792 | CA | LYS | D | 388 | 44.909 | 108.195 | 200.915 | 1.00 | 49.64 |
| 3793 | CB | LYS | D | 383 | 46.349 | 108.672 | 200.653 | 1.00 | 19.49 |
| 3794 | CG | LYS | D | 388 | 47.490 | 107.749 | 201.074 | 1.00 | 148.66 |
| 3795 | CD | LYS | D | 388 | 48.839 | 108.366 | 200.675 | 1.00 | 145.63 |
| 3796 | CE | LYS | D | 388 | 50.016 | 107.483 | 201.069 | 1.00 | 169.40 |
| 3797 | NZ | LYS | D | 388 | 51.321 | 108.173 | 200.844 | 1.00 | 149.37 |
| 3798 | C | LYS | D | 388 | 44.068 | 108.854 | 199.854 | 1.00 | 40.50 |
| 3799 | O | LYS | D | 388 | 44.011 | 108.358 | 198.735 | 1.00 | 60.66 |
| 3800 | N | GLU | D | 389 | 43.425 | 109.971 | 200.183 | 1.00 | 34.31 |
| 3801 | CA | GLU | D | 389 | 42.645 | 110.713 | 199.183 | 1.00 | 51.61 |
| 3802 | CB | GLU | D | 389 | 41.143 | 110.672 | 199.478 | 1.00 | 15.19 |
| 3803 | CG | GLU | D | 389 | 40.520 | 109.280 | 199.334 | 1.00 | 140.73 |
| 3804 | CD | GLU | D | 389 | 39.070 | 109.211 | 199.796 | 1.00 | 168.49 |
| 3805 | OE1 | GLU | D | 389 | 38.263 | 110.060 | 199.354 | 1.00 | 149.55 |
| 3806 | OE2 | GLU | D | 389 | 38.741 | 108.300 | 200.594 | 1.00 | 151.07 |
| 3807 | C | GLU | D | 389 | 43.118 | 112.159 | 199.147 | 1.00 | 49.41 |
| 3808 | O | GLU | D | 389 | 42.719 | 112.969 | 199.973 | 1.00 | 85.28 |
| 3809 | N | GLU | D | 390 | 43.966 | 112.478 | 198.178 | 1.00 | 36.48 |
| 3810 | CA | GLU | D | 390 | 44.525 | 113.817 | 198.052 | 1.00 | 75.79 |
| 3811 | CB | GLU | D | 390 | 46.005 | 113.748 | 197.638 | 1.00 | 105.75 |
| 3812 | CG | GLU | D | 390 | 46.901 | 112.828 | 198.462 | 1.00 | 149.81 |
| 3813 | CD | GLU | D | 390 | 48.363 | 112.896 | 198.028 | 1.00 | 166.87 |
| 3814 | OE1 | GLU | D | 390 | 48.638 | 112.760 | 196.814 | 1.00 | 167.85 |
| 3815 | OE2 | GLU | D | 390 | 49.237 | 113.080 | 198.903 | 1.00 | 158.75 |
| 3816 | C | GLU | D | 390 | 43.826 | 114.727 | 197.055 | 1.00 | 24.22 |
| 3817 | O | GLU | D | 390 | 43.939 | 114.519 | 195.855 | 1.00 | 71.23 |
| 3818 | N | LYS | D | 391 | 43.126 | 115.746 | 197.535 | 1.00 | 39.14 |
| 3819 | CA | LYS | D | 391 | 42.485 | 116.689 | 196.620 | 1.00 | 57.00 |
| 3820 | CB | LYS | D | 391 | 41.731 | 117.794 | 197.388 | 1.00 | 45.82 |
| 3821 | CG | LYS | D | 391 | 41.324 | 118.992 | 196.519 | 1.00 | 82.77 |
| 3822 | CD | LYS | D | 391 | 41.375 | 120.325 | 197.271 | 1.00 | 142.55 |
| 3823 | CE | LYS | D | 391 | 40.285 | 120.453 | 198.331 | 1.00 | 170.58 |
| 3824 | NZ | LYS | D | 391 | 40.286 | 121.807 | 198.979 | 1.00 | 121.90 |
| 3825 | C | LYS | D | 391 | 43.652 | 117.317 | 195.856 | 1.00 | 48.81 |
| 3826 | O | LYS | D | 391 | 44.550 | 117.872 | 196.476 | 1.00 | 51.69 |
| 3827 | N | GLN | D | 392 | 43.649 | 117.218 | 194.526 | 1.00 | 65.02 |
| 3828 | CA | GLN | D | 392 | 44.730 | 117.787 | 193.725 | 1.00 | 59.17 |
| 3829 | CB | GLN | D | 392 | 44.920 | 116.991 | 192.448 | 1.00 | 65.61 |
| 3830 | CG | GLN | D | 392 | 45.169 | 115.537 | 192.684 | 1.00 | 54.54 |
| 3831 | CD | GLN | D | 392 | 46.454 | 115.307 | 193.399 | 1.00 | 38.88 |
| 3832 | OE1 | GLN | D | 392 | 46.604 | 115.681 | 194.556 | 1.00 | 126.60 |
| 3833 | NE2 | GLN | D | 392 | 47.412 | 114.693 | 192.712 | 1.00 | 107.57 |
| 3834 | C | GLN | D | 392 | 44.461 | 119.237 | 193.368 | 1.00 | 88.60 |
| 3835 | O | GLN | D | 392 | 43.348 | 119.743 | 193.559 | 1.00 | 64.57 |
| 3836 | N | ARG | D | 393 | 45.487 | 119.896 | 192.835 | 1.00 | 88.23 |
| 3837 | CA | ARG | D | 393 | 45.381 | 121.299 | 192.463 | 1.00 | 103.72 |
| 3838 | CB | ARG | D | 393 | 46.747 | 121.855 | 192.045 | 1.00 | 134.66 |
| 3839 | CG | ARG | D | 393 | 46.697 | 123.320 | 191.590 | 1.00 | 159.94 |
| 3840 | CD | ARG | D | 393 | 47.397 | 124.271 | 192.559 | 1.00 | 160.67 |
| 3841 | NE | ARG | D | 393 | 48.846 | 124.077 | 192.564 | 1.00 | 168.47 |
| 3842 | CZ | ARG | D | 393 | 49.702 | 124.859 | 193.212 | 1.00 | 172.58 |
| 3843 | NH1 | ARG | D | 393 | 49.257 | 125.894 | 193.913 | 1.00 | 174.62 |
| 3844 | NH2 | ARG | D | 393 | 51.004 | 124.606 | 193.160 | 1.00 | 174.39 |
| 3845 | C | ARG | D | 393 | 44.382 | 121.561 | 191.351 | 1.00 | 83.46 |
| 3846 | O | ARG | D | 393 | 43.633 | 122.543 | 191.414 | 1.00 | 96.92 |
| 3847 | N | ASN | D | 394 | 44.357 | 120.694 | 190.338 | 1.00 | 93.61 |
| 3848 | CA | ASN | D | 394 | 43.437 | 120.897 | 189.219 | 1.00 | 95.98 |
| 3849 | CB | ASN | D | 394 | 43.924 | 120.138 | 187.969 | 1.00 | 83.40 |
| 3850 | CG | ASN | D | 394 | 43.957 | 118.632 | 188.154 | 1.00 | 48.87 |
| 3851 | OD1 | ASN | D | 394 | 43.110 | 118.070 | 188.828 | 1.00 | 66.78 |
| 3852 | ND2 | ASN | D | 394 | 44.927 | 117.982 | 187.516 | 1.00 | 75.04 |
| 3853 | C | ASN | D | 394 | 41.952 | 120.583 | 189.479 | 1.00 | 59.45 |
| 3854 | O | ASN | D | 394 | 41.227 | 120.189 | 188.572 | 1.00 | 59.33 |
| 3855 | N | GLY | D | 395 | 41.494 | 120.775 | 190.710 | 1.00 | 79.33 |
| 3856 | CA | GLY | D | 395 | 40.099 | 120.509 | 191.008 | 1.00 | 71.67 |
| 3857 | C | GLY | D | 395 | 39.724 | 119.038 | 191.108 | 1.00 | 59.87 |
| 3858 | O | GLY | D | 395 | 38.677 | 118.700 | 191.669 | 1.00 | 74.71 |
| 3859 | N | THR | D | 396 | 40.564 | 118.157 | 190.574 | 1.00 | 62.74 |
| 3860 | CA | THR | D | 396 | 40.274 | 116.730 | 190.626 | 1.00 | 61.60 |
| 3861 | CB | THR | D | 396 | 41.206 | 115.932 | 189.705 | 1.00 | 47.87 |
| 3862 | OG1 | THR | D | 396 | 40.702 | 114.600 | 189.558 | 1.00 | 156.72 |
| 3863 | CG2 | THR | D | 396 | 42.599 | 115.848 | 190.305 | 1.00 | 112.24 |
| 3864 | C | THR | D | 396 | 40.436 | 116.198 | 192.048 | 1.00 | 53.86 |
| 3865 | O | THR | D | 396 | 40.495 | 116.964 | 193.001 | 1.00 | 71.70 |
| 3866 | N | LEU | D | 397 | 40.520 | 114.878 | 192.171 | 1.00 | 45.95 |
| 3867 | CA | LEU | D | 397 | 40.655 | 114.203 | 193.454 | 1.00 | 53.38 |
| 3868 | CB | LEU | D | 397 | 39.281 | 114.097 | 194.110 | 1.00 | 59.95 |
| 3869 | CG | LEU | D | 397 | 39.121 | 113.074 | 195.221 | 1.00 | 55.86 |
| 3870 | CD1 | LEU | D | 397 | 40.080 | 113.397 | 196.345 | 1.00 | 111.38 |
| 3871 | CD2 | LEU | D | 397 | 37.685 | 113.061 | 195.710 | 1.00 | 106.37 |
| 3872 | C | LEU | D | 397 | 41.232 | 112.809 | 193.198 | 1.00 | 50.48 |
| 3873 | O | LEU | D | 397 | 40.675 | 112.039 | 192.417 | 1.00 | 62.36 |
| 3874 | N | THR | D | 398 | 42.337 | 112.493 | 193.868 | 1.00 | 40.39 |
| 3875 | CA | THR | D | 398 | 43.020 | 111.224 | 193.706 | 1.00 | 21.97 |
| 3876 | CB | THR | D | 398 | 44.512 | 111.441 | 193.543 | 1.00 | 25.83 |
| 3877 | OG1 | THR | D | 398 | 44.756 | 112.125 | 192.315 | 1.00 | 66.94 |
| 3878 | CG2 | THR | D | 398 | 45.246 | 110.136 | 193.527 | 1.00 | 67.01 |
| 3879 | C | THR | D | 398 | 42.844 | 110.303 | 194.865 | 1.00 | 24.03 |
| 3880 | O | THR | D | 398 | 42.586 | 110.737 | 195.973 | 1.00 | 59.86 |
| 3881 | N | VAL | D | 399 | 43.008 | 109.015 | 194.602 | 1.00 | 37.39 |
| 3882 | CA | VAL | D | 399 | 42.909 | 107.980 | 195.628 | 1.00 | 37.07 |
| 3883 | CB | VAL | D | 399 | 41.574 | 107.254 | 195.564 | 1.00 | 32.64 |
| 3884 | CG1 | VAL | D | 399 | 41.609 | 106.041 | 196.480 | 1.00 | 32.87 |
| 3885 | CG2 | VAL | D | 399 | 40.476 | 108.176 | 195.946 | 1.00 | 28.21 |
| 3886 | C | VAL | D | 399 | 43.973 | 106.932 | 195.385 | 1.00 | 41.18 |
| 3887 | O | VAL | D | 399 | 44.031 | 106.372 | 194.293 | 1.00 | 45.84 |
| 3888 | N | THR | D | 400 | 44.818 | 106.664 | 196.375 | 1.00 | 50.23 |
| 3889 | CA | THR | D | 400 | 45.834 | 105.625 | 196.202 | 1.00 | 38.12 |
| 3890 | CB | THR | D | 400 | 47.270 | 106.128 | 196.319 | 1.00 | 43.92 |
| 3891 | OG1 | THR | D | 400 | 47.494 | 106.558 | 197.655 | 1.00 | 69.44 |
| 3892 | CG2 | THR | D | 400 | 47.526 | 107.280 | 195.375 | 1.00 | 45.17 |
| 3893 | C | THR | D | 400 | 45.648 | 104.626 | 197.309 | 1.00 | 35.51 |
| 3894 | O | THR | D | 400 | 45.223 | 104.968 | 198.404 | 1.00 | 64.95 |
| 3895 | N | SER | D | 401 | 45.969 | 103.382 | 197.016 | 1.00 | 42.58 |
| 3896 | CA | SER | D | 401 | 45.840 | 102.318 | 197.990 | 1.00 | 42.11 |
| 3897 | CB | SER | D | 401 | 44.515 | 101.584 | 197.833 | 1.00 | 59.45 |
| 3898 | OG | SER | D | 401 | 44.482 | 100.421 | 198.643 | 1.00 | 51.13 |
| 3899 | C | SER | D | 401 | 46.946 | 101.344 | 197.735 | 1.00 | 49.43 |
| 3900 | O | SER | D | 401 | 47.042 | 100.807 | 196.641 | 1.00 | 49.62 |
| 3901 | N | THR | D | 402 | 47.779 | 101.118 | 198.742 | 1.00 | 72.73 |
| 3902 | CA | THR | D | 402 | 48.894 | 100.196 | 198.624 | 1.00 | 50.60 |
| 3903 | CB | THR | D | 402 | 50.150 | 100.813 | 199.187 | 1.00 | 50.34 |
| 3904 | OG1 | THR | D | 402 | 50.322 | 102.117 | 198.630 | 1.00 | 61.02 |
| 3905 | CG2 | THR | D | 402 | 51.345 | 99.975 | 198.836 | 1.00 | 108.37 |
| 3906 | C | THR | D | 402 | 48.571 | 98.928 | 199.394 | 1.00 | 59.31 |
| 3907 | O | THR | D | 402 | 47.951 | 98.978 | 200.447 | 1.00 | 73.63 |
| 3908 | N | LEU | D | 403 | 48.995 | 97.790 | 198.872 | 1.00 | 60.48 |
| 3909 | CA | LEU | D | 403 | 48.701 | 96.520 | 199.510 | 1.00 | 51.40 |
| 3910 | CB | LEU | D | 403 | 47.648 | 95.785 | 198.689 | 1.00 | 60.86 |
| 3911 | CG | LEU | D | 403 | 47.239 | 94.398 | 199.166 | 1.00 | 71.08 |
| 3912 | CD1 | LEU | D | 403 | 46.519 | 94.506 | 200.492 | 1.00 | 115.39 |
| 3913 | CD2 | LEU | D | 403 | 46.339 | 93.757 | 198.140 | 1.00 | 75.16 |
| 3914 | C | LEU | D | 403 | 49.919 | 95.624 | 199.684 | 1.00 | 86.73 |
| 3915 | O | LEU | D | 403 | 50.684 | 95.411 | 198.741 | 1.00 | 48.79 |
| 3916 | N | PRO | D | 404 | 50.113 | 95.090 | 200.901 | 1.00 | 45.77 |
| 3917 | CD | PRO | D | 404 | 49.349 | 95.460 | 202.098 | 1.00 | 72.52 |
| 3918 | CA | PRO | D | 404 | 51.215 | 94.203 | 201.270 | 1.00 | 60.92 |
| 3919 | CB | PRO | D | 404 | 51.070 | 94.080 | 202.784 | 1.00 | 110.61 |
| 3920 | CG | PRO | D | 404 | 50.390 | 95.345 | 203.168 | 1.00 | 84.70 |
| 3921 | C | PRO | D | 404 | 51.030 | 92.861 | 200.573 | 1.00 | 78.31 |
| 3922 | O | PRO | D | 404 | 49.926 | 92.321 | 200.528 | 1.00 | 62.25 |
| 3923 | N | VAL | D | 405 | 52.116 | 92.304 | 200.055 | 1.00 | 85.38 |
| 3924 | CA | VAL | D | 405 | 52.015 | 91.056 | 199.330 | 1.00 | 72.08 |
| 3925 | CB | VAL | D | 405 | 52.032 | 91.358 | 197.832 | 1.00 | 42.37 |
| 3926 | CG1 | VAL | D | 405 | 53.051 | 90.497 | 197.120 | 1.00 | 129.72 |
| 3927 | CG2 | VAL | D | 405 | 50.651 | 91.145 | 197.266 | 1.00 | 97.10 |
| 3928 | C | VAL | D | 405 | 53.063 | 90.002 | 199.661 | 1.00 | 107.38 |
| 3929 | O | VAL | D | 405 | 54.263 | 90.289 | 199.721 | 1.00 | 92.57 |
| 3930 | N | GLY | D | 406 | 52.584 | 88.772 | 199.849 | 1.00 | 74.73 |
| 3931 | CA | GLY | D | 406 | 53.459 | 87.656 | 200.171 | 1.00 | 128.36 |
| 3932 | C | GLY | D | 406 | 54.503 | 87.396 | 199.084 | 1.00 | 128.26 |
| 3933 | O | GLY | D | 406 | 54.170 | 86.932 | 197.995 | 1.00 | 94.96 |
| 3934 | N | THR | D | 407 | 55.763 | 87.675 | 199.410 | 1.00 | 145.41 |
| 3935 | CA | THR | D | 407 | 56.880 | 87.505 | 198.508 | 1.00 | 103.78 |
| 3936 | CB | THR | D | 407 | 58.220 | 87.513 | 199.231 | 1.00 | 128.76 |
| 3937 | OG1 | THR | D | 407 | 58.109 | 88.244 | 200.452 | 1.00 | 153.49 |
| 3938 | CG2 | THR | D | 407 | 59.289 | 88.139 | 198.329 | 1.00 | 97.34 |
| 3939 | C | THR | D | 407 | 56.852 | 86.189 | 197.741 | 1.00 | 107.15 |
| 3940 | O | THR | D | 407 | 57.225 | 86.126 | 196.572 | 1.00 | 95.02 |
| 3941 | N | ARG | D | 408 | 56.441 | 85.123 | 198.408 | 1.00 | 126.42 |
| 3942 | CA | ARG | D | 408 | 56.399 | 83.820 | 197.772 | 1.00 | 134.10 |
| 3943 | CB | ARG | D | 408 | 56.112 | 82.750 | 198.816 | 1.00 | 151.42 |
| 3944 | CG | ARG | D | 408 | 57.188 | 82.649 | 199.914 | 1.00 | 177.48 |
| 3945 | CD | ARG | D | 408 | 57.344 | 83.945 | 200.730 | 1.00 | 172.46 |
| 3946 | NE | ARG | D | 408 | 56.196 | 84.229 | 201.588 | 1.00 | 159.11 |
| 3947 | CZ | ARG | D | 408 | 56.079 | 85.320 | 202.337 | 1.00 | 143.95 |
| 3948 | NH1 | ARG | D | 408 | 57.038 | 86.240 | 202.332 | 1.00 | 95.33 |
| 3949 | NH2 | ARG | D | 408 | 55.013 | 85.480 | 203.107 | 1.00 | 128.49 |
| 3950 | C | ARG | D | 408 | 55.302 | 83.805 | 196.723 | 1.00 | 126.54 |
| 3951 | O | ARG | D | 408 | 55.556 | 83.753 | 195.505 | 1.00 | 112.58 |
| 3952 | N | ASP | D | 409 | 54.070 | 83.869 | 197.211 | 1.00 | 115.25 |
| 3953 | CA | ASP | D | 409 | 52.874 | 83.849 | 196.382 | 1.00 | 99.87 |
| 3954 | CB | ASP | D | 409 | 51.731 | 84.516 | 197.160 | 1.00 | 106.74 |
| 3955 | CG | ASP | D | 409 | 51.541 | 83.939 | 198.559 | 1.00 | 132.42 |
| 3956 | OD1 | ASP | D | 409 | 51.021 | 82.808 | 198.688 | 1.00 | 129.76 |
| 3957 | OD2 | ASP | D | 409 | 51.909 | 84.625 | 199.539 | 1.00 | 112.17 |
| 3958 | C | ASP | D | 409 | 53.053 | 84.546 | 195.032 | 1.00 | 89.51 |
| 3959 | O | ASP | D | 409 | 52.675 | 84.000 | 193.990 | 1.00 | 130.58 |
| 3960 | N | TRP | D | 410 | 53.622 | 85.749 | 195.061 | 1.00 | 75.82 |
| 3961 | CA | TRP | D | 410 | 53.836 | 86.534 | 193.853 | 1.00 | 67.87 |
| 3962 | CB | TRP | D | 410 | 54.487 | 87.863 | 194.209 | 1.00 | 73.93 |
| 3963 | CG | TRP | D | 410 | 54.924 | 88.660 | 193.025 | 1.00 | 51.99 |
| 3964 | CD2 | TRP | D | 410 | 54.138 | 89.608 | 192.297 | 1.00 | 89.75 |
| 3965 | CE2 | TRP | D | 410 | 54.954 | 90.123 | 191.266 | 1.00 | 74.90 |
| 3966 | CE3 | TRP | D | 410 | 52.822 | 90.071 | 192.416 | 1.00 | 51.02 |
| 3967 | CD1 | TRP | D | 410 | 56.145 | 88.636 | 192.423 | 1.00 | 101.45 |
| 3968 | NE1 | TRP | D | 410 | 56.174 | 89.515 | 191.365 | 1.00 | 51.04 |
| 3969 | CZ2 | TRP | D | 410 | 54.498 | 91.083 | 190.357 | 1.00 | 99.21 |
| 3970 | CZ3 | TRP | D | 410 | 52.369 | 91.020 | 191.518 | 1.00 | 50.44 |
| 3971 | CH2 | TRP | D | 410 | 53.208 | 91.520 | 190.496 | 1.00 | 72.30 |
| 3972 | C | TRP | D | 410 | 54.684 | 85.829 | 192.825 | 1.00 | 97.87 |
| 3973 | O | TRP | D | 410 | 54.246 | 85.630 | 191.693 | 1.00 | 127.37 |
| 3974 | N | ILE | D | 411 | 55.900 | 85.458 | 193.215 | 1.00 | 107.01 |
| 3975 | CA | ILE | D | 411 | 56.807 | 84.797 | 192.283 | 1.00 | 114.82 |
| 3976 | CB | ILE | D | 411 | 58.125 | 84.422 | 192.943 | 1.00 | 90.18 |
| 3977 | CG2 | ILE | D | 411 | 59.219 | 84.374 | 191.890 | 1.00 | 113.38 |
| 3978 | CG1 | ILE | D | 411 | 58.504 | 85.477 | 193.973 | 1.00 | 122.60 |
| 3979 | CD1 | ILE | D | 411 | 59.713 | 85.112 | 194.782 | 1.00 | 160.55 |
| 3980 | C | ILE | D | 411 | 56.182 | 83.539 | 191.713 | 1.00 | 131.40 |
| 3981 | O | ILE | D | 411 | 56.470 | 83.146 | 190.585 | 1.00 | 115.52 |
| 3982 | N | GLU | D | 412 | 55.322 | 82.903 | 192.497 | 1.00 | 92.44 |
| 3983 | CA | GLU | D | 412 | 54.655 | 81.704 | 192.031 | 1.00 | 108.96 |
| 3984 | CB | GLU | D | 412 | 54.287 | 80.823 | 193.216 | 1.00 | 133.49 |
| 3985 | CG | GLU | D | 412 | 55.489 | 80.102 | 193.793 | 1.00 | 154.48 |
| 3986 | CD | GLU | D | 412 | 55.112 | 79.134 | 194.888 | 1.00 | 184.63 |
| 3987 | OE1 | GLU | D | 412 | 54.194 | 78.315 | 194.668 | 1.00 | 187.73 |
| 3988 | OE2 | GLU | D | 412 | 55.738 | 79.187 | 195.967 | 1.00 | 191.09 |
| 3989 | C | GLU | D | 412 | 53.428 | 81.997 | 191.162 | 1.00 | 137.18 |
| 3990 | O | GLU | D | 412 | 52.457 | 81.236 | 191.149 | 1.00 | 129.46 |
| 3991 | N | GLY | D | 413 | 53.485 | 83.118 | 190.446 | 1.00 | 164.33 |
| 3992 | CA | GLY | D | 413 | 52.417 | 83.501 | 189.535 | 1.00 | 162.77 |
| 3993 | C | GLY | D | 413 | 51.059 | 83.957 | 190.041 | 1.00 | 141.45 |
| 3994 | O | GLY | D | 413 | 50.144 | 84.149 | 189.237 | 1.00 | 136.83 |
| 3995 | N | GLU | D | 414 | 50.903 | 84.135 | 191.347 | 1.00 | 129.17 |
| 3996 | CA | GLU | D | 414 | 49.620 | 84.580 | 191.869 | 1.00 | 99.45 |
| 3997 | CB | GLU | D | 414 | 49.731 | 84.925 | 193.349 | 1.00 | 110.96 |
| 3998 | CG | GLU | D | 414 | 48.471 | 85.542 | 193.936 | 1.00 | 90.67 |
| 3999 | CD | GLU | D | 414 | 47.256 | 84.634 | 193.834 | 1.00 | 94.32 |
| 4000 | OE1 | GLU | D | 414 | 46.758 | 84.399 | 192.709 | 1.00 | 112.62 |
| 4001 | OE2 | GLU | D | 414 | 46.802 | 84.152 | 194.891 | 1.00 | 91.31 |
| 4002 | C | GLU | D | 414 | 49.146 | 85.803 | 191.098 | 1.00 | 100.87 |
| 4003 | O | GLU | D | 414 | 49.917 | 86.424 | 190.368 | 1.00 | 107.15 |
| 4004 | N | THR | D | 415 | 47.876 | 86.149 | 191.266 | 1.00 | 111.06 |
| 4005 | CA | THR | D | 415 | 47.303 | 87.291 | 190.574 | 1.00 | 112.82 |
| 4006 | CB | THR | D | 415 | 46.590 | 86.825 | 189.295 | 1.00 | 119.50 |
| 4007 | OG1 | THR | D | 415 | 45.583 | 87.773 | 188.933 | 1.00 | 115.11 |
| 4008 | CG2 | THR | D | 415 | 45.993 | 85.452 | 189.487 | 1.00 | 150.67 |
| 4009 | C | THR | D | 415 | 46.348 | 88.115 | 191.442 | 1.00 | 127.64 |
| 4010 | O | THR | D | 415 | 45.360 | 87.595 | 191.982 | 1.00 | 91.30 |
| 4011 | N | TYR | D | 416 | 46.664 | 89.407 | 191.559 | 1.00 | 114.78 |
| 4012 | CA | TYR | D | 416 | 45.899 | 90.360 | 192.366 | 1.00 | 69.47 |
| 4013 | CB | TYR | D | 416 | 46.846 | 91.150 | 193.276 | 1.00 | 76.73 |
| 4014 | CG | TYR | D | 416 | 47.732 | 90.297 | 194.155 | 1.00 | 89.68 |
| 4015 | CD1 | TYR | D | 416 | 48.988 | 89.875 | 193.724 | 1.00 | 115.56 |
| 4016 | CE1 | TYR | D | 416 | 49.787 | 89.049 | 194.524 | 1.00 | 52.64 |
| 4017 | CD2 | TYR | D | 416 | 47.298 | 89.877 | 195.406 | 1.00 | 74.22 |
| 4018 | CE2 | TYR | D | 416 | 48.086 | 89.054 | 196.210 | 1.00 | 65.75 |
| 4019 | CZ | TYR | D | 416 | 49.321 | 88.645 | 195.766 | 1.00 | 79.16 |
| 4020 | OH | TYR | D | 416 | 50.081 | 87.831 | 196.571 | 1.00 | 157.74 |
| 4021 | C | TYR | D | 416 | 45.089 | 91.342 | 191.521 | 1.00 | 92.20 |
| 4022 | O | TYR | D | 416 | 45.521 | 91.745 | 190.438 | 1.00 | 69.13 |
| 4023 | N | GLN | D | 417 | 43.931 | 91.752 | 192.037 | 1.00 | 67.36 |
| 4024 | CA | GLN | D | 417 | 43.050 | 92.668 | 191.319 | 1.00 | 48.33 |
| 4025 | CB | GLN | D | 417 | 41.897 | 91.876 | 190.709 | 1.00 | 124.19 |
| 4026 | CG | GLN | D | 417 | 41.057 | 92.649 | 189.718 | 1.00 | 145.26 |
| 4027 | CD | GLN | D | 417 | 39.854 | 91.857 | 189.256 | 1.00 | 138.49 |
| 4028 | OE1 | GLN | D | 417 | 38.942 | 91.590 | 190.037 | 1.00 | 121.32 |
| 4029 | NE2 | GLN | D | 417 | 39.848 | 91.469 | 187.985 | 1.00 | 129.85 |
| 4030 | C | GLN | D | 417 | 42.484 | 93.795 | 192.191 | 1.00 | 57.83 |
| 4031 | O | GLN | D | 417 | 42.091 | 93.579 | 193.338 | 1.00 | 77.99 |
| 4032 | N | CYS | D | 418 | 42.426 | 94.992 | 191.614 | 1.00 | 66.84 |
| 4033 | CA | CYS | D | 418 | 41.930 | 96.191 | 192.286 | 1.00 | 48.93 |
| 4034 | C | CYS | D | 418 | 40.585 | 96.549 | 191.670 | 1.00 | 61.50 |
| 4035 | O | CYS | D | 418 | 40.496 | 96.739 | 190.465 | 1.00 | 67.84 |
| 4036 | CB | CYS | D | 418 | 42.927 | 97.336 | 192.079 | 1.00 | 66.08 |
| 4037 | SG | CYS | D | 418 | 42.483 | 98.904 | 192.868 | 1.00 | 95.99 |
| 4038 | N | ARG | D | 419 | 39.541 | 96.638 | 192.490 | 1.00 | 50.79 |
| 4039 | CA | ARG | D | 419 | 38.201 | 96.969 | 191.992 | 1.00 | 30.24 |
| 4040 | CB | ARG | D | 419 | 37.226 | 95.822 | 192.284 | 1.00 | 108.73 |
| 4041 | CG | ARG | D | 419 | 35.835 | 96.025 | 191.697 | 1.00 | 188.65 |
| 4042 | CD | ARG | D | 419 | 34.860 | 94.941 | 192.125 | 1.00 | 228.92 |
| 4043 | NE | ARG | D | 419 | 33.561 | 95.099 | 191.473 | 1.00 | 243.94 |
| 4044 | CZ | ARG | D | 419 | 32.495 | 94.354 | 191.738 | 1.00 | 230.39 |
| 4045 | NH1 | ARG | D | 419 | 32.566 | 93.394 | 192.649 | 1.00 | 220.71 |
| 4046 | NH2 | ARG | D | 419 | 31.359 | 94.565 | 191.090 | 1.00 | 214.39 |
| 4047 | C | ARG | D | 419 | 37.661 | 98.255 | 192.610 | 1.00 | 69.76 |
| 4048 | O | ARG | D | 419 | 37.299 | 98.287 | 193.797 | 1.00 | 59.29 |
| 4049 | N | VAL | D | 420 | 37.597 | 99.315 | 191.809 | 1.00 | 18.00 |
| 4050 | CA | VAL | D | 420 | 37.074 | 100.578 | 192.324 | 1.00 | 56.50 |
| 4051 | CB | VAL | D | 420 | 37.934 | 101.774 | 191.866 | 1.00 | 52.22 |
| 4052 | CG1 | VAL | D | 420 | 39.318 | 101.315 | 191.544 | 1.00 | 42.61 |
| 4053 | CG2 | VAL | D | 420 | 37.327 | 102.441 | 190.689 | 1.00 | 35.59 |
| 4054 | C | VAL | D | 420 | 35.665 | 100.745 | 191.782 | 1.00 | 35.00 |
| 4055 | O | VAL | D | 420 | 35.385 | 100.297 | 190.700 | 1.00 | 37.99 |
| 4056 | N | THR | D | 421 | 34.772 | 101.395 | 192.502 | 1.00 | 56.57 |
| 4057 | CA | THR | D | 421 | 33.425 | 101.562 | 191.982 | 1.00 | 23.71 |
| 4058 | CB | THR | D | 421 | 32.465 | 100.554 | 192.642 | 1.00 | 45.48 |
| 4059 | OG1 | THR | D | 421 | 32.418 | 100.794 | 194.052 | 1.00 | 67.88 |
| 4060 | CG2 | THR | D | 421 | 32.941 | 99.141 | 192.431 | 1.00 | 34.10 |
| 4061 | C | THR | D | 421 | 32.981 | 102.971 | 192.327 | 1.00 | 45.43 |
| 4062 | O | THR | D | 421 | 33.240 | 103.446 | 193.427 | 1.00 | 48.61 |
| 4063 | N | HIS | D | 422 | 32.334 | 103.649 | 191.389 | 1.00 | 43.04 |
| 4064 | CA | HIS | D | 422 | 31.840 | 105.009 | 191.627 | 1.00 | 50.66 |
| 4065 | CB | HIS | D | 422 | 32.559 | 106.002 | 190.718 | 1.00 | 14.59 |
| 4066 | CG | HIS | D | 422 | 32.154 | 107.422 | 190.931 | 1.00 | 21.53 |
| 4067 | CD2 | HIS | D | 422 | 32.698 | 108.580 | 190.485 | 1.00 | 65.88 |
| 4068 | ND1 | HIS | D | 422 | 31.052 | 107.779 | 191.675 | 1.00 | 53.72 |
| 4069 | CE1 | HIS | D | 422 | 30.935 | 109.095 | 191.681 | 1.00 | 114.73 |
| 4070 | NE2 | HIS | D | 422 | 31.922 | 109.606 | 190.966 | 1.00 | 87.07 |
| 4071 | C | HIS | D | 422 | 30.344 | 105.008 | 191.319 | 1.00 | 73.12 |
| 4072 | O | HIS | D | 422 | 29.920 | 104.610 | 190.236 | 1.00 | 67.37 |
| 4073 | N | PRO | D | 423 | 29.523 | 105.465 | 192.265 | 1.00 | 58.08 |
| 4074 | CD | PRO | D | 423 | 29.888 | 106.227 | 193.469 | 1.00 | 54.31 |
| 4075 | CA | PRO | D | 423 | 28.076 | 105.492 | 192.059 | 1.00 | 71.43 |
| 4076 | CB | PRO | D | 423 | 27.608 | 106.488 | 193.108 | 1.00 | 46.51 |
| 4077 | CG | PRO | D | 423 | 28.588 | 106.285 | 194.211 | 1.00 | 82.86 |
| 4078 | C | PRO | D | 423 | 27.619 | 105.884 | 190.654 | 1.00 | 63.28 |
| 4079 | O | PRO | D | 423 | 26.833 | 105.173 | 190.028 | 1.00 | 81.37 |
| 4080 | N | HIS | D | 424 | 28.123 | 107.005 | 190.157 | 1.00 | 34.90 |
| 4081 | CA | HIS | D | 424 | 27.730 | 107.502 | 188.848 | 1.00 | 59.77 |
| 4082 | CB | HIS | D | 424 | 28.010 | 108.999 | 188.754 | 1.00 | 37.61 |
| 4083 | CG | HIS | D | 424 | 27.748 | 109.743 | 190.019 | 1.00 | 60.94 |
| 4084 | CD2 | HIS | D | 424 | 27.244 | 109.336 | 191.207 | 1.00 | 98.42 |
| 4085 | ND1 | HIS | D | 424 | 28.068 | 111.072 | 190.174 | 1.00 | 78.59 |
| 4086 | CE1 | HIS | D | 424 | 27.778 | 111.452 | 191.406 | 1.00 | 152.64 |
| 4087 | NE2 | HIS | D | 424 | 27.277 | 110.417 | 192.054 | 1.00 | 152.52 |
| 4088 | C | HIS | D | 424 | 28.388 | 106.814 | 187.654 | 1.00 | 36.89 |
| 4089 | O | HIS | D | 424 | 28.568 | 107.433 | 186.612 | 1.00 | 54.52 |
| 4090 | N | LEU | D | 425 | 28.758 | 105.548 | 187.776 | 1.00 | 21.15 |
| 4091 | CA | LEU | D | 425 | 29.371 | 104.884 | 186.632 | 1.00 | 37.06 |
| 4092 | CB | LEU | D | 425 | 30.857 | 104.611 | 186.893 | 1.00 | 56.98 |
| 4093 | CG | LEU | D | 425 | 31.771 | 105.825 | 187.067 | 1.00 | 23.50 |
| 4094 | CD1 | LEU | D | 425 | 33.179 | 105.368 | 187.366 | 1.00 | 58.60 |
| 4095 | CD2 | LEU | D | 425 | 31.746 | 106.677 | 185.827 | 1.00 | 24.67 |
| 4096 | C | LEU | D | 425 | 28.680 | 103.584 | 186.237 | 1.00 | 31.46 |
| 4097 | O | LEU | D | 425 | 28.161 | 102.858 | 187.077 | 1.00 | 64.74 |
| 4098 | N | PRO | D | 426 | 28.660 | 103.287 | 184.936 | 1.00 | 23.81 |
| 4099 | CD | PRO | D | 426 | 29.067 | 104.203 | 183.862 | 1.00 | 71.59 |
| 4100 | CA | PRO | D | 426 | 28.053 | 102.088 | 184.376 | 1.00 | 41.36 |
| 4101 | CB | PRO | D | 426 | 28.260 | 102.281 | 182.885 | 1.00 | 29.87 |
| 4102 | CG | PRO | D | 426 | 28.215 | 103.739 | 182.734 | 1.00 | 66.78 |
| 4103 | C | PRO | D | 426 | 28.735 | 100.834 | 184.912 | 1.00 | 26.52 |
| 4104 | O | PRO | D | 426 | 28.315 | 100.289 | 185.916 | 1.00 | 55.27 |
| 4105 | N | ARG | D | 427 | 29.785 | 100.369 | 184.249 | 1.00 | 73.53 |
| 4106 | CA | ARG | D | 427 | 30.472 | 99.182 | 184.725 | 1.00 | 63.70 |
| 4107 | CB | ARG | D | 427 | 31.083 | 98.404 | 183.541 | 1.00 | 62.24 |
| 4108 | CG | ARG | D | 427 | 30.616 | 96.941 | 183.493 | 1.00 | 74.02 |
| 4109 | CD | ARG | D | 427 | 31.214 | 96.127 | 182.354 | 1.00 | 70.65 |
| 4110 | NE | ARG | D | 427 | 31.336 | 94.711 | 182.718 | 1.00 | 159.98 |
| 4111 | CZ | ARG | D | 427 | 31.878 | 93.762 | 181.953 | 1.00 | 164.46 |
| 4112 | NH1 | ARG | D | 427 | 32.363 | 94.046 | 180.749 | 1.00 | 124.37 |
| 4113 | NH2 | ARG | D | 427 | 31.951 | 92.516 | 182.405 | 1.00 | 133.62 |
| 4114 | C | ARG | D | 427 | 31.532 | 99.618 | 185.751 | 1.00 | 86.41 |
| 4115 | O | ARG | D | 427 | 31.880 | 100.800 | 185.828 | 1.00 | 47.25 |
| 4116 | N | ALA | D | 428 | 32.013 | 98.674 | 186.559 | 1.00 | 93.41 |
| 4117 | CA | ALA | D | 428 | 33.005 | 98.970 | 187.595 | 1.00 | 41.66 |
| 4118 | CB | ALA | D | 428 | 32.956 | 97.912 | 188.669 | 1.00 | 102.75 |
| 4119 | C | ALA | D | 428 | 34.400 | 99.058 | 187.028 | 1.00 | 45.06 |
| 4120 | O | ALA | D | 428 | 34.674 | 98.543 | 185.958 | 1.00 | 75.62 |
| 4121 | N | LEU | D | 429 | 35.291 | 99.721 | 187.744 | 1.00 | 66.10 |
| 4122 | CA | LEU | D | 429 | 36.641 | 99.848 | 187.242 | 1.00 | 43.45 |
| 4123 | CB | LEU | D | 429 | 37.222 | 101.217 | 187.556 | 1.00 | 34.38 |
| 4124 | CG | LEU | D | 429 | 37.745 | 101.978 | 186.364 | 1.00 | 66.23 |
| 4125 | CD1 | LEU | D | 429 | 38.458 | 103.214 | 186.845 | 1.00 | 46.80 |
| 4126 | CD2 | LEU | D | 429 | 38.672 | 101.084 | 185.569 | 1.00 | 115.61 |
| 4127 | C | LEU | D | 429 | 37.484 | 98.785 | 187.887 | 1.00 | 54.85 |
| 4128 | O | LEU | D | 429 | 37.496 | 98.631 | 189.117 | 1.00 | 59.59 |
| 4129 | N | MET | D | 430 | 38.184 | 98.040 | 187.043 | 1.00 | 47.43 |
| 4130 | CA | MET | D | 430 | 39.057 | 96.962 | 187.494 | 1.00 | 53.01 |
| 4131 | CB | MET | D | 430 | 38.412 | 95.594 | 187.284 | 1.00 | 40.35 |
| 4132 | CG | MET | D | 430 | 37.179 | 95.375 | 188.128 | 1.00 | 77.82 |
| 4133 | SD | MET | D | 430 | 36.525 | 93.713 | 187.975 | 1.00 | 126.20 |
| 4134 | CE | MET | D | 430 | 35.574 | 93.859 | 186.419 | 1.00 | 161.71 |
| 4135 | C | MET | D | 430 | 40.366 | 96.986 | 186.745 | 1.00 | 63.35 |
| 4136 | O | MET | D | 430 | 40.448 | 97.480 | 185.612 | 1.00 | 42.39 |
| 4137 | N | ARG | D | 431 | 41.392 | 96.466 | 187.407 | 1.00 | 36.33 |
| 4138 | CA | ARG | D | 431 | 42.719 | 96.379 | 186.841 | 1.00 | 58.15 |
| 4139 | CB | ARG | D | 431 | 43.464 | 97.696 | 186.993 | 1.00 | 27.19 |
| 4140 | CG | ARG | D | 431 | 42.683 | 98.900 | 186.503 | 1.00 | 33.53 |
| 4141 | CD | ARG | D | 431 | 43.552 | 99.896 | 185.806 | 1.00 | 43.03 |
| 4142 | NE | ARG | D | 431 | 43.438 | 99.788 | 184.355 | 1.00 | 95.31 |
| 4143 | CZ | ARG | D | 431 | 42.328 | 100.067 | 183.679 | 1.00 | 104.17 |
| 4144 | NH1 | ARG | D | 431 | 41.245 | 100.468 | 184.330 | 1.00 | 37.36 |
| 4145 | NH2 | ARG | D | 431 | 42.300 | 99.949 | 182.355 | 1.00 | 165.84 |
| 4146 | C | ARG | D | 431 | 43.363 | 95.299 | 187.666 | 1.00 | 67.38 |
| 4147 | O | ARG | D | 431 | 43.050 | 95.151 | 188.839 | 1.00 | 56.89 |
| 4148 | N | SER | D | 432 | 44.240 | 94.527 | 187.046 | 1.00 | 77.67 |
| 4149 | CA | SER | D | 432 | 44.897 | 93.431 | 187.734 | 1.00 | 64.82 |
| 4150 | CB | SER | D | 432 | 44.192 | 92.118 | 187.410 | 1.00 | 56.74 |
| 4151 | OG | SER | D | 432 | 44.290 | 91.820 | 186.027 | 1.00 | 99.74 |
| 4152 | C | SER | D | 432 | 46.343 | 93.350 | 187.291 | 1.00 | 94.30 |
| 4153 | O | SER | D | 432 | 46.754 | 94.039 | 186.355 | 1.00 | 64.60 |
| 4154 | N | THR | D | 433 | 47.112 | 92.507 | 187.969 | 1.00 | 73.10 |
| 4155 | CA | THR | D | 433 | 48.519 | 92.339 | 187.647 | 1.00 | 82.03 |
| 4156 | CB | THR | D | 433 | 49.341 | 93.538 | 188.076 | 1.00 | 73.97 |
| 4157 | OG1 | THR | D | 433 | 50.696 | 93.368 | 187.640 | 1.00 | 108.43 |
| 4158 | CG2 | THR | D | 433 | 49.314 | 93.664 | 189.589 | 1.00 | 50.90 |
| 4159 | C | THR | D | 433 | 49.110 | 91.140 | 188.340 | 1.00 | 97.97 |
| 4160 | O | THR | D | 433 | 48.687 | 90.772 | 189.450 | 1.00 | 54.76 |
| 4161 | N | THR | D | 434 | 50.101 | 90.543 | 187.673 | 1.00 | 88.14 |
| 4162 | CA | THR | D | 434 | 50.835 | 89.377 | 188.176 | 1.00 | 61.31 |
| 4163 | CB | THR | D | 434 | 50.212 | 88.050 | 187.757 | 1.00 | 73.67 |
| 4164 | OG1 | THR | D | 434 | 50.604 | 87.723 | 186.414 | 1.00 | 136.86 |
| 4165 | CG2 | THR | D | 434 | 48.713 | 88.120 | 187.813 | 1.00 | 65.49 |
| 4166 | C | THR | D | 434 | 52.219 | 89.389 | 187.548 | 1.00 | 93.47 |
| 4167 | O | THR | D | 434 | 52.500 | 90.185 | 186.661 | 1.00 | 68.59 |
| 4168 | N | LYS | D | 435 | 53.052 | 88.481 | 188.041 | 1.00 | 104.82 |
| 4169 | CA | LYS | D | 435 | 54.425 | 88.280 | 187.620 | 1.00 | 97.19 |
| 4170 | CB | LYS | D | 435 | 54.913 | 86.952 | 188.190 | 1.00 | 108.47 |
| 4171 | CG | LYS | D | 435 | 56.289 | 86.406 | 187.805 | 1.00 | 156.78 |
| 4172 | CD | LYS | D | 435 | 56.494 | 85.099 | 188.627 | 1.00 | 153.33 |
| 4173 | CE | LYS | D | 435 | 57.777 | 84.278 | 188.369 | 1.00 | 171.23 |
| 4174 | NZ | LYS | D | 435 | 57.958 | 83.976 | 186.919 | 1.00 | 182.18 |
| 4175 | C | LYS | D | 435 | 54.554 | 88.263 | 186.100 | 1.00 | 93.46 |
| 4176 | O | LYS | D | 435 | 53.814 | 87.555 | 185.412 | 1.00 | 120.14 |
| 4177 | N | THR | D | 436 | 55.489 | 89.042 | 185.566 | 1.00 | 71.73 |
| 4178 | CA | THR | D | 436 | 55.677 | 89.079 | 184.118 | 1.00 | 82.60 |
| 4179 | CB | THR | D | 436 | 56.715 | 90.111 | 183.748 | 1.00 | 75.56 |
| 4180 | OG1 | THR | D | 436 | 56.400 | 91.352 | 184.383 | 1.00 | 131.33 |
| 4181 | CG2 | THR | D | 436 | 56.746 | 90.317 | 182.254 | 1.00 | 83.66 |
| 4182 | C | THR | D | 436 | 56.132 | 87.718 | 183.594 | 1.00 | 127.18 |
| 4183 | O | THR | D | 436 | 56.943 | 87.046 | 184.224 | 1.00 | 131.75 |
| 4184 | N | SER | D | 437 | 55.651 | 87.344 | 182.413 | 1.00 | 129.98 |
| 4185 | CA | SER | D | 437 | 55.976 | 86.043 | 181.824 | 1.00 | 150.36 |
| 4186 | CB | SER | D | 437 | 54.732 | 85.467 | 181.140 | 1.00 | 156.11 |
| 4187 | OG | SER | D | 437 | 54.249 | 86.345 | 180.132 | 1.00 | 150.74 |
| 4188 | C | SER | D | 437 | 57.136 | 86.032 | 180.844 | 1.00 | 145.33 |
| 4189 | O | SER | D | 437 | 57.791 | 87.049 | 180.633 | 1.00 | 128.92 |
| 4190 | N | GLY | D | 438 | 57.385 | 84.861 | 180.261 | 1.00 | 143.82 |
| 4191 | CA | GLY | D | 438 | 58.455 | 84.708 | 179.289 | 1.00 | 129.59 |
| 4192 | C | GLY | D | 438 | 59.692 | 83.989 | 179.804 | 1.00 | 109.80 |
| 4193 | O | GLY | D | 438 | 59.641 | 83.339 | 180.849 | 1.00 | 90.90 |
| 4194 | N | PRO | D | 439 | 60.814 | 84.057 | 179.066 | 1.00 | 96.86 |
| 4195 | CD | PRO | D | 439 | 60.853 | 84.528 | 177.672 | 1.00 | 102.41 |
| 4196 | CA | PRO | D | 439 | 62.088 | 83.431 | 179.427 | 1.00 | 112.86 |
| 4197 | CB | PRO | D | 439 | 62.754 | 83.224 | 178.079 | 1.00 | 143.69 |
| 4198 | CG | PRO | D | 439 | 62.322 | 84.442 | 177.338 | 1.00 | 120.60 |
| 4199 | C | PRO | D | 439 | 62.861 | 84.406 | 180.312 | 1.00 | 120.63 |
| 4200 | O | PRO | D | 439 | 62.881 | 85.600 | 180.044 | 1.00 | 129.73 |
| 4201 | N | ARG | D | 440 | 63.503 | 83.905 | 181.357 | 1.00 | 110.84 |
| 4202 | CA | ARG | D | 440 | 64.231 | 84.774 | 182.276 | 1.00 | 110.52 |
| 4203 | CB | ARG | D | 440 | 64.025 | 84.289 | 183.711 | 1.00 | 128.23 |
| 4204 | CG | ARG | D | 440 | 62.627 | 83.749 | 184.002 | 1.00 | 153.84 |
| 4205 | CD | ARG | D | 440 | 61.579 | 84.849 | 184.102 | 1.00 | 188.47 |
| 4206 | NE | ARG | D | 440 | 60.232 | 84.332 | 183.856 | 1.00 | 215.81 |
| 4207 | CZ | ARG | D | 440 | 59.122 | 84.806 | 184.410 | 1.00 | 224.86 |
| 4208 | NH1 | ARG | D | 440 | 59.180 | 85.822 | 185.260 | 1.00 | 223.24 |
| 4209 | NH2 | ARG | D | 440 | 57.954 | 84.257 | 184.108 | 1.00 | 209.34 |
| 4210 | C | ARG | D | 440 | 65.726 | 84.853 | 181.978 | 1.00 | 83.55 |
| 4211 | O | ARG | D | 440 | 66.468 | 83.937 | 182.310 | 1.00 | 129.96 |
| 4212 | N | ALA | D | 441 | 66.172 | 85.951 | 181.372 | 1.00 | 74.53 |
| 4213 | CA | ALA | D | 441 | 67.589 | 86.119 | 181.054 | 1.00 | 78.06 |
| 4214 | CB | ALA | D | 441 | 67.753 | 86.473 | 179.593 | 1.00 | 98.44 |
| 4215 | C | ALA | D | 441 | 68.222 | 87.195 | 181.922 | 1.00 | 73.89 |
| 4216 | O | ALA | D | 441 | 67.781 | 88.340 | 181.908 | 1.00 | 87.18 |
| 4217 | N | ALA | D | 442 | 69.267 | 86.818 | 182.659 | 1.00 | 108.00 |
| 4218 | CA | ALA | D | 442 | 69.993 | 87.722 | 183.560 | 1.00 | 84.59 |
| 4219 | CB | ALA | D | 442 | 71.104 | 86.959 | 184.254 | 1.00 | 124.78 |
| 4220 | C | ALA | D | 442 | 70.561 | 88.989 | 182.906 | 1.00 | 90.01 |
| 4221 | O | ALA | D | 442 | 70.706 | 89.070 | 181.687 | 1.00 | 88.98 |
| 4222 | N | PRO | D | 443 | 70.904 | 89.993 | 183.725 | 1.00 | 81.16 |
| 4223 | CD | PRO | D | 443 | 70.625 | 90.055 | 185.162 | 1.00 | 58.29 |
| 4224 | CA | PRO | D | 443 | 71.449 | 91.273 | 183.272 | 1.00 | 65.44 |
| 4225 | CB | PRO | D | 443 | 71.044 | 92.255 | 184.377 | 1.00 | 62.14 |
| 4226 | CG | PRO | D | 443 | 70.143 | 91.467 | 185.294 | 1.00 | 84.05 |
| 4227 | C | PRO | D | 443 | 72.943 | 91.293 | 183.088 | 1.00 | 72.84 |
| 4228 | O | PRO | D | 443 | 73.682 | 90.606 | 183.789 | 1.00 | 105.86 |
| 4229 | N | GLU | D | 444 | 73.376 | 92.113 | 182.145 | 1.00 | 79.19 |
| 4230 | CA | GLU | D | 444 | 74.782 | 92.292 | 181.855 | 1.00 | 104.43 |
| 4231 | CB | GLU | D | 444 | 75.063 | 92.051 | 180.370 | 1.00 | 143.96 |
| 4232 | CG | GLU | D | 444 | 75.062 | 90.591 | 179.943 | 1.00 | 170.26 |
| 4233 | CD | GLU | D | 444 | 74.905 | 90.425 | 178.443 | 1.00 | 152.52 |
| 4234 | OE1 | GLU | D | 444 | 75.481 | 91.243 | 177.693 | 1.00 | 116.93 |
| 4235 | OE2 | GLU | D | 444 | 74.210 | 89.475 | 178.018 | 1.00 | 138.72 |
| 4236 | C | GLU | D | 444 | 74.992 | 93.747 | 182.180 | 1.00 | 84.83 |
| 4237 | O | GLU | D | 444 | 74.317 | 94.602 | 181.616 | 1.00 | 95.04 |
| 4238 | N | VAL | D | 445 | 75.902 | 94.043 | 183.097 | 1.00 | 87.28 |
| 4239 | CA | VAL | D | 445 | 76.152 | 95.433 | 183.453 | 1.00 | 76.03 |
| 4240 | CB | VAL | D | 445 | 75.736 | 95.706 | 184.897 | 1.00 | 75.27 |
| 4241 | CG1 | VAL | D | 445 | 76.170 | 94.563 | 185.778 | 1.00 | 68.09 |
| 4242 | CG2 | VAL | D | 445 | 76.333 | 97.020 | 185.366 | 1.00 | 93.41 |
| 4243 | C | VAL | D | 445 | 77.599 | 95.863 | 183.264 | 1.00 | 60.53 |
| 4244 | O | VAL | D | 445 | 78.520 | 95.096 | 183.545 | 1.00 | 92.85 |
| 4245 | N | TYR | D | 446 | 77.791 | 97.090 | 182.783 | 1.00 | 65.90 |
| 4246 | CA | TYR | D | 446 | 79.128 | 97.633 | 182.546 | 1.00 | 97.21 |
| 4247 | CB | TYR | D | 446 | 79.483 | 97.555 | 181.054 | 1.00 | 115.92 |
| 4248 | CG | TYR | D | 446 | 79.187 | 96.214 | 180.416 | 1.00 | 124.25 |
| 4249 | CD1 | TYR | D | 446 | 78.088 | 96.048 | 179.579 | 1.00 | 108.68 |
| 4250 | CE1 | TYR | D | 446 | 77.774 | 94.803 | 179.036 | 1.00 | 143.50 |
| 4251 | CD2 | TYR | D | 446 | 79.975 | 95.097 | 180.692 | 1.00 | 175.81 |
| 4252 | CE2 | TYR | D | 446 | 79.671 | 93.845 | 180.153 | 1.00 | 164.42 |
| 4253 | CZ | TYR | D | 446 | 78.568 | 93.707 | 179.329 | 1.00 | 149.93 |
| 4254 | OH | TYR | D | 446 | 78.246 | 92.473 | 178.813 | 1.00 | 162.97 |
| 4255 | C | TYR | D | 446 | 79.143 | 99.086 | 182.995 | 1.00 | 97.06 |
| 4256 | O | TYR | D | 446 | 78.386 | 99.896 | 182.470 | 1.00 | 99.04 |
| 4257 | N | ALA | D | 447 | 80.005 | 99.415 | 183.956 | 1.00 | 102.47 |
| 4258 | CA | ALA | D | 447 | 80.100 | 100.782 | 184.484 | 1.00 | 66.24 |
| 4259 | CB | ALA | D | 447 | 80.300 | 100.730 | 185.984 | 1.00 | 89.68 |
| 4260 | C | ALA | D | 447 | 81.211 | 101.615 | 183.841 | 1.00 | 84.25 |
| 4261 | O | ALA | D | 447 | 82.290 | 101.102 | 183.544 | 1.00 | 81.08 |
| 4262 | N | PHE | D | 448 | 80.957 | 102.906 | 183.642 | 1.00 | 53.71 |
| 4263 | CA | PHE | D | 448 | 81.955 | 103.767 | 183.018 | 1.00 | 96.40 |
| 4264 | CB | PHE | D | 448 | 81.528 | 104.081 | 181.583 | 1.00 | 110.89 |
| 4265 | CG | PHE | D | 448 | 81.356 | 102.852 | 180.729 | 1.00 | 139.15 |
| 4266 | CD1 | PHE | D | 448 | 80.213 | 102.073 | 180.831 | 1.00 | 147.96 |
| 4267 | CD2 | PHE | D | 448 | 82.368 | 102.438 | 179.872 | 1.00 | 185.33 |
| 4268 | CE1 | PHE | D | 448 | 80.080 | 100.901 | 180.100 | 1.00 | 149.26 |
| 4269 | CE2 | PHE | D | 448 | 82.246 | 101.267 | 179.135 | 1.00 | 184.09 |
| 4270 | CZ | PHE | D | 448 | 81.099 | 100.497 | 179.250 | 1.00 | 182.71 |
| 4271 | C | PHE | D | 448 | 82.240 | 105.047 | 183.799 | 1.00 | 83.02 |
| 4272 | O | PHE | D | 448 | 81.778 | 105.196 | 184.921 | 1.00 | 92.98 |
| 4273 | N | ALA | D | 449 | 83.011 | 105.963 | 183.216 | 1.00 | 86.20 |
| 4274 | CA | ALA | D | 449 | 83.363 | 107.210 | 183.901 | 1.00 | 46.82 |
| 4275 | CB | ALA | D | 449 | 84.286 | 106.910 | 185.063 | 1.00 | 99.13 |
| 4276 | C | ALA | D | 449 | 84.019 | 108.243 | 182.987 | 1.00 | 66.70 |
| 4277 | O | ALA | D | 449 | 85.007 | 107.964 | 182.302 | 1.00 | 82.28 |
| 4278 | N | THR | D | 450 | 83.478 | 109.452 | 183.017 | 1.00 | 46.07 |
| 4279 | CA | THR | D | 450 | 83.953 | 110.551 | 182.182 | 1.00 | 90.49 |
| 4280 | CB | THR | D | 450 | 82.895 | 111.677 | 182.176 | 1.00 | 66.00 |
| 4281 | OG1 | THR | D | 450 | 81.684 | 111.176 | 181.611 | 1.00 | 102.05 |
| 4282 | CG2 | THR | D | 450 | 83.355 | 112.870 | 181.373 | 1.00 | 103.25 |
| 4283 | C | THR | D | 450 | 85.298 | 111.169 | 182.568 | 1.00 | 89.86 |
| 4284 | O | THR | D | 450 | 85.722 | 111.083 | 183.711 | 1.00 | 116.46 |
| 4285 | N | PRO | D | 451 | 86.004 | 111.760 | 181.590 | 1.00 | 87.34 |
| 4286 | CD | PRO | D | 451 | 85.899 | 111.307 | 180.196 | 1.00 | 73.15 |
| 4287 | CA | PRO | D | 451 | 87.298 | 112.421 | 181.800 | 1.00 | 105.99 |
| 4288 | CB | PRO | D | 451 | 87.997 | 112.253 | 180.450 | 1.00 | 127.70 |
| 4289 | CG | PRO | D | 451 | 87.335 | 111.052 | 179.859 | 1.00 | 115.43 |
| 4290 | C | PRO | D | 451 | 86.971 | 113.891 | 182.083 | 1.00 | 101.34 |
| 4291 | O | PRO | D | 451 | 85.922 | 114.375 | 181.670 | 1.00 | 107.89 |
| 4292 | N | GLU | D | 452 | 87.850 | 114.611 | 182.768 | 1.00 | 115.89 |
| 4293 | CA | GLU | D | 452 | 87.560 | 116.009 | 183.060 | 1.00 | 133.25 |
| 4294 | CB | GLU | D | 452 | 88.700 | 116.645 | 183.863 | 1.00 | 159.83 |
| 4295 | CG | GLU | D | 452 | 90.046 | 116.683 | 183.155 | 1.00 | 194.80 |
| 4296 | CD | GLU | D | 452 | 91.097 | 117.446 | 183.947 | 1.00 | 196.79 |
| 4297 | OE1 | GLU | D | 452 | 90.906 | 118.663 | 184.170 | 1.00 | 177.79 |
| 4298 | OE2 | GLU | D | 452 | 92.110 | 116.830 | 184.347 | 1.00 | 195.58 |
| 4299 | C | GLU | D | 452 | 87.294 | 116.834 | 181.801 | 1.00 | 120.67 |
| 4300 | O | GLU | D | 452 | 87.544 | 116.392 | 180.677 | 1.00 | 86.09 |
| 4301 | N | TRP | D | 453 | 86.776 | 118.039 | 182.007 | 1.00 | 123.90 |
| 4302 | CA | TRP | D | 453 | 86.462 | 118.953 | 180.916 | 1.00 | 145.95 |
| 4303 | CB | TRP | D | 453 | 84.992 | 118.777 | 180.504 | 1.00 | 170.02 |
| 4304 | CG | TRP | D | 453 | 84.500 | 119.744 | 179.459 | 1.00 | 197.51 |
| 4305 | CD2 | TRP | D | 453 | 84.449 | 119.528 | 178.042 | 1.00 | 214.10 |
| 4306 | CE2 | TRP | D | 453 | 83.925 | 120.703 | 177.457 | 1.00 | 215.96 |
| 4307 | CE3 | TRP | D | 453 | 84.792 | 118.453 | 177.210 | 1.00 | 219.72 |
| 4308 | CD1 | TRP | D | 453 | 84.022 | 121.006 | 179.668 | 1.00 | 209.18 |
| 4309 | NE1 | TRP | D | 453 | 83.675 | 121.589 | 178.471 | 1.00 | 210.81 |
| 4310 | CZ2 | TRP | D | 453 | 83.741 | 120.838 | 176.076 | 1.00 | 220.86 |
| 4311 | CZ3 | TRP | D | 453 | 84.609 | 118.587 | 175.834 | 1.00 | 220.01 |
| 4312 | CH2 | TRP | D | 453 | 84.086 | 119.772 | 175.284 | 1.00 | 223.85 |
| 4313 | C | TRP | D | 453 | 86.731 | 120.387 | 181.372 | 1.00 | 158.31 |
| 4314 | O | TRP | D | 453 | 86.448 | 120.748 | 182.517 | 1.00 | 155.59 |
| 4315 | N | PRO | D | 454 | 87.295 | 121.221 | 180.480 | 1.00 | 164.35 |
| 4316 | CD | PRO | D | 454 | 87.673 | 120.851 | 179.102 | 1.00 | 146.83 |
| 4317 | CA | PRO | D | 454 | 87.624 | 122.628 | 180.746 | 1.00 | 184.14 |
| 4318 | CB | PRO | D | 454 | 87.864 | 123.187 | 179.348 | 1.00 | 181.33 |
| 4319 | CG | PRO | D | 454 | 88.523 | 122.033 | 178.664 | 1.00 | 173.35 |
| 4320 | C | PRO | D | 454 | 86.558 | 123.418 | 181.509 | 1.00 | 195.45 |
| 4321 | O | PRO | D | 454 | 86.855 | 124.447 | 182.116 | 1.00 | 210.90 |
| 4322 | N | GLY | D | 455 | 85.321 | 122.934 | 181.474 | 1.00 | 201.41 |
| 4323 | CA | GLY | D | 455 | 84.239 | 123.613 | 182.165 | 1.00 | 189.27 |
| 4324 | C | GLY | D | 455 | 84.373 | 123.555 | 183.674 | 1.00 | 182.16 |
| 4325 | O | GLY | D | 455 | 84.142 | 124.550 | 184.362 | 1.00 | 191.75 |
| 4326 | N | SER | D | 456 | 84.746 | 122.388 | 184.189 | 1.00 | 171.47 |
| 4327 | CA | SER | D | 456 | 84.913 | 122.193 | 185.625 | 1.00 | 166.50 |
| 4328 | CB | SER | D | 456 | 83.591 | 121.774 | 186.261 | 1.00 | 158.47 |
| 4329 | OG | SER | D | 456 | 83.188 | 120.511 | 185.764 | 1.00 | 140.37 |
| 4330 | C | SER | D | 456 | 85.943 | 121.101 | 185.858 | 1.00 | 166.83 |
| 4331 | O | SER | D | 456 | 85.996 | 120.122 | 185.114 | 1.00 | 174.49 |
| 4332 | N | ARG | D | 457 | 86.751 | 121.264 | 186.899 | 1.00 | 175.34 |
| 4333 | CA | ARG | D | 457 | 87.787 | 120.288 | 187.213 | 1.00 | 178.26 |
| 4334 | CB | ARG | D | 457 | 89.137 | 121.000 | 187.387 | 1.00 | 185.27 |
| 4335 | CG | ARG | D | 457 | 89.546 | 121.846 | 186.184 | 1.00 | 203.14 |
| 4336 | CD | ARG | D | 457 | 90.908 | 122.507 | 186.373 | 1.00 | 206.79 |
| 4337 | NE | ARG | D | 457 | 91.246 | 123.379 | 185.247 | 1.00 | 216.85 |
| 4338 | CZ | ARG | D | 457 | 92.393 | 124.041 | 185.125 | 1.00 | 205.77 |
| 4339 | NH1 | ARG | D | 457 | 93.328 | 123.937 | 186.060 | 1.00 | 207.14 |
| 4340 | NH2 | ARG | D | 457 | 92.603 | 124.814 | 184.068 | 1.00 | 185.77 |
| 4341 | C | ARG | D | 457 | 87.468 | 119.472 | 188.464 | 1.00 | 162.65 |
| 4342 | O | ARG | D | 457 | 88.291 | 118.675 | 188.915 | 1.00 | 168.22 |
| 4343 | N | ASP | D | 458 | 86.274 | 119.660 | 189.018 | 1.00 | 149.08 |
| 4344 | CA | ASP | D | 458 | 85.890 | 118.934 | 190.226 | 1.00 | 166.05 |
| 4345 | CB | ASP | D | 458 | 85.908 | 119.875 | 191.434 | 1.00 | 188.82 |
| 4346 | CG | ASP | D | 458 | 87.300 | 120.382 | 191.754 | 1.00 | 202.29 |
| 4347 | OD1 | ASP | D | 458 | 88.212 | 119.544 | 191.919 | 1.00 | 202.70 |
| 4348 | OD2 | ASP | D | 458 | 87.480 | 121.615 | 191.843 | 1.00 | 197.86 |
| 4349 | C | ASP | D | 458 | 84.531 | 118.249 | 190.137 | 1.00 | 167.63 |
| 4350 | O | ASP | D | 458 | 83.930 | 117.904 | 191.158 | 1.00 | 106.20 |
| 4351 | N | LYS | D | 459 | 84.055 | 118.053 | 188.912 | 1.00 | 184.87 |
| 4352 | CA | LYS | D | 459 | 82.773 | 117.395 | 188.672 | 1.00 | 167.70 |
| 4353 | CB | LYS | D | 459 | 81.697 | 118.424 | 188.295 | 1.00 | 177.88 |
| 4354 | CG | LYS | D | 459 | 81.395 | 119.467 | 189.373 | 1.00 | 201.66 |
| 4355 | CD | LYS | D | 459 | 80.275 | 120.423 | 188.944 | 1.00 | 187.25 |
| 4356 | CE | LYS | D | 459 | 79.958 | 121.446 | 190.038 | 1.00 | 173.49 |
| 4357 | NZ | LYS | D | 459 | 78.840 | 122.362 | 189.668 | 1.00 | 145.56 |
| 4358 | C | LYS | D | 459 | 82.932 | 116.382 | 187.538 | 1.00 | 165.68 |
| 4359 | O | LYS | D | 459 | 83.276 | 116.748 | 186.410 | 1.00 | 172.64 |
| 4360 | N | ARG | D | 460 | 82.697 | 115.109 | 187.843 | 1.00 | 154.79 |
| 4361 | CA | ARG | D | 460 | 82.804 | 114.051 | 186.841 | 1.00 | 153.22 |
| 4362 | CB | ARG | D | 460 | 84.095 | 113.244 | 187.058 | 1.00 | 125.90 |
| 4363 | CG | ARG | D | 460 | 85.358 | 114.037 | 186.706 | 1.00 | 137.80 |
| 4364 | CD | ARG | D | 460 | 86.588 | 113.151 | 186.537 | 1.00 | 147.84 |
| 4365 | NE | ARG | D | 460 | 87.717 | 113.902 | 185.989 | 1.00 | 154.85 |
| 4366 | CZ | ARG | D | 460 | 88.857 | 113.355 | 185.572 | 1.00 | 177.19 |
| 4367 | NH1 | ARG | D | 460 | 89.037 | 112.042 | 185.634 | 1.00 | 158.76 |
| 4368 | NH2 | ARG | D | 460 | 89.824 | 114.124 | 185.090 | 1.00 | 193.89 |
| 4369 | C | ARG | D | 460 | 81.561 | 113.141 | 186.828 | 1.00 | 141.33 |
| 4370 | O | ARG | D | 460 | 81.033 | 112.758 | 187.876 | 1.00 | 118.12 |
| 4371 | N | THR | D | 461 | 81.116 | 112.794 | 185.623 | 1.00 | 106.56 |
| 4372 | CA | THR | D | 461 | 79.912 | 111.998 | 185.408 | 1.00 | 72.86 |
| 4373 | CB | THR | D | 461 | 79.214 | 112.496 | 184.135 | 1.00 | 85.08 |
| 4374 | OG1 | THR | D | 461 | 79.268 | 113.928 | 184.100 | 1.00 | 106.27 |
| 4375 | CG2 | THR | D | 461 | 77.771 | 112.026 | 184.092 | 1.00 | 59.78 |
| 4376 | C | THR | D | 461 | 80.058 | 110.484 | 185.278 | 1.00 | 72.76 |
| 4377 | O | THR | D | 461 | 80.940 | 110.002 | 184.571 | 1.00 | 64.16 |
| 4378 | N | LEU | D | 462 | 79.174 | 109.739 | 185.941 | 1.00 | 67.15 |
| 4379 | CA | LEU | D | 462 | 79.176 | 108.269 | 185.838 | 1.00 | 93.90 |
| 4380 | CB | LEU | D | 462 | 79.119 | 107.581 | 187.202 | 1.00 | 54.53 |
| 4381 | CG | LEU | D | 462 | 80.338 | 107.682 | 188.115 | 1.00 | 86.30 |
| 4382 | CD1 | LEU | D | 462 | 80.306 | 106.517 | 189.095 | 1.00 | 67.24 |
| 4383 | CD2 | LEU | D | 462 | 81.621 | 107.639 | 187.305 | 1.00 | 77.87 |
| 4384 | C | LEU | D | 462 | 77.991 | 107.767 | 185.029 | 1.00 | 86.86 |
| 4385 | O | LEU | D | 462 | 76.949 | 108.418 | 184.974 | 1.00 | 135.96 |
| 4386 | N | ALA | D | 463 | 78.158 | 106.600 | 184.416 | 1.00 | 84.07 |
| 4387 | CA | ALA | D | 463 | 77.117 | 105.982 | 183.597 | 1.00 | 70.48 |
| 4388 | CB | ALA | D | 463 | 77.311 | 106.345 | 182.132 | 1.00 | 69.52 |
| 4389 | C | ALA | D | 463 | 77.176 | 104.476 | 183.773 | 1.00 | 66.61 |
| 4390 | O | ALA | D | 463 | 78.250 | 103.897 | 183.916 | 1.00 | 72.40 |
| 4391 | N | CYS | D | 464 | 76.021 | 103.837 | 183.750 | 1.00 | 69.61 |
| 4392 | CA | CYS | D | 464 | 75.980 | 102.407 | 183.946 | 1.00 | 72.19 |
| 4393 | C | CYS | D | 464 | 74.947 | 101.799 | 183.022 | 1.00 | 95.42 |
| 4394 | O | CYS | D | 464 | 73.769 | 102.144 | 183.080 | 1.00 | 118.74 |
| 4395 | CB | CYS | D | 464 | 75.636 | 102.132 | 185.398 | 1.00 | 50.48 |
| 4396 | SG | CYS | D | 464 | 75.624 | 100.393 | 185.919 | 1.00 | 118.72 |
| 4397 | N | LEU | D | 465 | 75.400 | 100.890 | 182.167 | 1.00 | 95.81 |
| 4398 | CA | LEU | D | 465 | 74.530 | 100.233 | 181.207 | 1.00 | 80.91 |
| 4399 | CB | LEU | D | 465 | 75.167 | 100.288 | 179.824 | 1.00 | 62.17 |
| 4400 | CG | LEU | D | 465 | 74.700 | 99.281 | 178.772 | 1.00 | 81.47 |
| 4401 | CD1 | LEU | D | 465 | 73.187 | 99.193 | 178.728 | 1.00 | 87.06 |
| 4402 | CD2 | LEU | D | 465 | 75.254 | 99.711 | 177.427 | 1.00 | 84.82 |
| 4403 | C | LEU | D | 465 | 74.198 | 98.791 | 181.565 | 1.00 | 79.74 |
| 4404 | O | LEU | D | 465 | 75.083 | 97.957 | 181.726 | 1.00 | 54.90 |
| 4405 | N | ILE | D | 466 | 72.906 | 98.509 | 181.669 | 1.00 | 64.80 |
| 4406 | CA | ILE | D | 466 | 72.437 | 97.182 | 182.007 | 1.00 | 58.32 |
| 4407 | CB | ILE | D | 466 | 71.567 | 97.232 | 183.254 | 1.00 | 77.30 |
| 4408 | CG2 | ILE | D | 466 | 71.276 | 95.823 | 183.747 | 1.00 | 58.46 |
| 4409 | CG1 | ILE | D | 466 | 72.281 | 98.066 | 184.316 | 1.00 | 44.32 |
| 4410 | CD1 | ILE | D | 466 | 71.633 | 98.043 | 185.670 | 1.00 | 83.41 |
| 4411 | C | ILE | D | 466 | 71.624 | 96.670 | 180.837 | 1.00 | 93.77 |
| 4412 | O | ILE | D | 466 | 70.586 | 97.243 | 180.502 | 1.00 | 98.80 |
| 4413 | N | GLN | D | 467 | 72.087 | 95.590 | 180.215 | 1.00 | 97.61 |
| 4414 | CA | GLN | D | 467 | 71.385 | 95.063 | 179.060 | 1.00 | 69.11 |
| 4415 | CB | GLN | D | 467 | 72.115 | 95.487 | 177.796 | 1.00 | 36.98 |
| 4416 | CG | GLN | D | 467 | 73.590 | 95.168 | 177.789 | 1.00 | 80.17 |
| 4417 | CD | GLN | D | 467 | 74.224 | 95.357 | 176.419 | 1.00 | 115.95 |
| 4418 | OE1 | GLN | D | 467 | 74.035 | 96.388 | 175.759 | 1.00 | 80.89 |
| 4419 | NE2 | GLN | D | 467 | 74.989 | 94.359 | 175.986 | 1.00 | 133.96 |
| 4420 | C | GLN | D | 467 | 71.124 | 93.570 | 178.994 | 1.00 | 43.59 |
| 4421 | O | GLN | D | 467 | 71.414 | 92.816 | 179.917 | 1.00 | 51.62 |
| 4422 | N | ASN | D | 468 | 70.542 | 93.175 | 177.868 | 1.00 | 84.41 |
| 4423 | CA | ASN | D | 468 | 70.204 | 91.797 | 177.562 | 1.00 | 78.87 |
| 4424 | CB | ASN | D | 468 | 71.448 | 91.071 | 177.072 | 1.00 | 91.58 |
| 4425 | CG | ASN | D | 468 | 72.167 | 91.847 | 175.992 | 1.00 | 131.22 |
| 4426 | OD1 | ASN | D | 468 | 71.557 | 92.267 | 175.000 | 1.00 | 109.30 |
| 4427 | ND2 | ASN | D | 468 | 73.467 | 92.052 | 176.179 | 1.00 | 130.02 |
| 4428 | C | ASN | D | 468 | 69.571 | 91.050 | 178.709 | 1.00 | 94.97 |
| 4429 | O | ASN | D | 468 | 70.049 | 89.988 | 179.113 | 1.00 | 113.94 |
| 4430 | N | PHE | D | 469 | 68.487 | 91.609 | 179.236 | 1.00 | 56.14 |
| 4431 | CA | PHE | D | 469 | 67.783 | 90.960 | 180.327 | 1.00 | 70.60 |
| 4432 | CB | PHE | D | 469 | 68.001 | 91.710 | 181.643 | 1.00 | 43.17 |
| 4433 | CG | PHE | D | 469 | 67.537 | 93.134 | 181.619 | 1.00 | 94.42 |
| 4434 | CD1 | PHE | D | 469 | 66.376 | 93.509 | 182.276 | 1.00 | 47.15 |
| 4435 | CD2 | PHE | D | 469 | 68.280 | 94.110 | 180.968 | 1.00 | 95.42 |
| 4436 | CE1 | PHE | D | 469 | 65.962 | 94.836 | 182.290 | 1.00 | 103.25 |
| 4437 | CE2 | PHE | D | 469 | 67.877 | 95.441 | 180.978 | 1.00 | 28.33 |
| 4438 | CZ | PHE | D | 469 | 66.716 | 95.804 | 181.639 | 1.00 | 101.58 |
| 4439 | C | PHE | D | 469 | 66.308 | 90.888 | 180.020 | 1.00 | 67.94 |
| 4440 | O | PHE | D | 469 | 65.834 | 91.535 | 179.083 | 1.00 | 51.85 |
| 4441 | N | MET | D | 470 | 65.600 | 90.078 | 180.805 | 1.00 | 27.30 |
| 4442 | CA | MET | D | 470 | 64.158 | 89.910 | 180.673 | 1.00 | 69.57 |
| 4443 | CB | MET | D | 470 | 63.814 | 89.320 | 179.308 | 1.00 | 108.06 |
| 4444 | CG | MET | D | 470 | 64.665 | 88.140 | 178.901 | 1.00 | 121.33 |
| 4445 | SD | MET | D | 470 | 64.817 | 88.077 | 177.103 | 1.00 | 132.64 |
| 4446 | CE | MET | D | 470 | 63.162 | 87.566 | 176.645 | 1.00 | 170.43 |
| 4447 | C | MET | D | 470 | 63.637 | 89.020 | 181.785 | 1.00 | 73.39 |
| 4448 | O | MET | D | 470 | 64.295 | 88.068 | 182.173 | 1.00 | 72.89 |
| 4449 | N | PRO | D | 471 | 62.445 | 89.328 | 182.328 | 1.00 | 77.22 |
| 4450 | CD | PRO | D | 471 | 61.921 | 88.464 | 183.393 | 1.00 | 79.25 |
| 4451 | CA | PRO | D | 471 | 61.500 | 90.415 | 182.042 | 1.00 | 73.03 |
| 4452 | CB | PRO | D | 471 | 60.443 | 90.228 | 183.117 | 1.00 | 77.74 |
| 4453 | CG | PRO | D | 471 | 60.456 | 88.748 | 183.334 | 1.00 | 95.61 |
| 4454 | C | PRO | D | 471 | 62.108 | 91.805 | 182.094 | 1.00 | 66.54 |
| 4455 | O | PRO | D | 471 | 63.298 | 91.962 | 182.323 | 1.00 | 79.62 |
| 4456 | N | GLU | D | 472 | 61.280 | 92.819 | 181.886 | 1.00 | 87.24 |
| 4457 | CA | GLU | D | 472 | 61.768 | 94.188 | 181.907 | 1.00 | 83.72 |
| 4458 | CB | GLU | D | 472 | 60.933 | 95.063 | 180.969 | 1.00 | 104.73 |
| 4459 | CG | GLU | D | 472 | 59.432 | 95.001 | 181.206 | 1.00 | 166.05 |
| 4460 | CD | GLU | D | 472 | 58.686 | 96.116 | 180.490 | 1.00 | 187.22 |
| 4461 | OE1 | GLU | D | 472 | 58.879 | 96.275 | 179.267 | 1.00 | 172.24 |
| 4462 | OE2 | GLU | D | 472 | 57.904 | 96.834 | 181.149 | 1.00 | 193.81 |
| 4463 | C | GLU | D | 472 | 61.770 | 94.796 | 183.304 | 1.00 | 104.32 |
| 4464 | O | GLU | D | 472 | 62.261 | 95.906 | 183.487 | 1.00 | 102.37 |
| 4465 | N | ASP | D | 473 | 61.233 | 94.077 | 184.288 | 1.00 | 59.56 |
| 4466 | CA | ASP | D | 473 | 61.187 | 94.593 | 185.649 | 1.00 | 92.12 |
| 4467 | CB | ASP | D | 473 | 60.234 | 93.756 | 186.504 | 1.00 | 116.46 |
| 4468 | CG | ASP | D | 473 | 58.789 | 93.929 | 186.094 | 1.00 | 135.25 |
| 4469 | OD1 | ASP | D | 473 | 58.358 | 95.090 | 185.944 | 1.00 | 150.62 |
| 4470 | OD2 | ASP | D | 473 | 58.086 | 92.910 | 185.927 | 1.00 | 137.23 |
| 4471 | C | ASP | D | 473 | 62.564 | 94.620 | 186.291 | 1.00 | 79.36 |
| 4472 | O | ASP | D | 473 | 63.061 | 93.592 | 186.742 | 1.00 | 60.87 |
| 4473 | N | ILE | D | 474 | 63.163 | 95.805 | 186.359 | 1.00 | 77.86 |
| 4474 | CA | ILE | D | 474 | 64.493 | 95.947 | 186.935 | 1.00 | 57.62 |
| 4475 | CB | ILE | D | 474 | 65.541 | 96.119 | 185.823 | 1.00 | 72.84 |
| 4476 | CG2 | ILE | D | 474 | 65.559 | 97.554 | 185.331 | 1.00 | 52.57 |
| 4477 | CG1 | ILE | D | 474 | 66.933 | 95.793 | 186.346 | 1.00 | 77.16 |
| 4478 | CD1 | ILE | D | 474 | 68.003 | 95.929 | 185.283 | 1.00 | 68.46 |
| 4479 | C | ILE | D | 474 | 64.646 | 97.117 | 187.900 | 1.00 | 75.73 |
| 4480 | O | ILE | D | 474 | 64.083 | 98.194 | 187.689 | 1.00 | 80.08 |
| 4481 | N | SER | D | 475 | 65.427 | 96.893 | 188.952 | 1.00 | 67.18 |
| 4482 | CA | SER | D | 475 | 65.698 | 97.914 | 189.957 | 1.00 | 72.99 |
| 4483 | CB | SER | D | 475 | 65.258 | 97.429 | 191.351 | 1.00 | 63.48 |
| 4484 | OG | SER | D | 475 | 63.999 | 97.964 | 191.727 | 1.00 | 93.55 |
| 4485 | C | SER | D | 475 | 67.188 | 98.261 | 189.979 | 1.00 | 53.66 |
| 4486 | O | SER | D | 475 | 68.017 | 97.470 | 190.419 | 1.00 | 75.03 |
| 4487 | N | VAL | D | 476 | 67.521 | 99.448 | 189.499 | 1.00 | 55.61 |
| 4488 | CA | VAL | D | 476 | 68.902 | 99.906 | 189.483 | 1.00 | 51.07 |
| 4489 | CB | VAL | D | 476 | 69.166 | 100.775 | 188.240 | 1.00 | 63.18 |
| 4490 | CG1 | VAL | D | 476 | 70.539 | 101.424 | 188.313 | 1.00 | 61.47 |
| 4491 | CG2 | VAL | D | 476 | 69.062 | 99.927 | 187.005 | 1.00 | 79.72 |
| 4492 | C | VAL | D | 476 | 69.166 | 100.749 | 190.728 | 1.00 | 58.51 |
| 4493 | O | VAL | D | 476 | 68.232 | 101.248 | 191.346 | 1.00 | 116.08 |
| 4494 | N | GLN | D | 477 | 70.436 | 100.908 | 191.085 | 1.00 | 70.31 |
| 4495 | CA | GLN | D | 477 | 70.840 | 101.706 | 192.242 | 1.00 | 86.47 |
| 4496 | CB | GLN | D | 477 | 70.317 | 101.076 | 193.526 | 1.00 | 59.72 |
| 4497 | CG | GLN | D | 477 | 70.614 | 99.603 | 193.661 | 1.00 | 61.26 |
| 4498 | CD | GLN | D | 477 | 69.958 | 99.013 | 194.889 | 1.00 | 111.88 |
| 4499 | OE1 | GLN | D | 477 | 68.738 | 99.086 | 195.044 | 1.00 | 120.03 |
| 4500 | NE2 | GLN | D | 477 | 70.761 | 98.429 | 195.775 | 1.00 | 97.73 |
| 4501 | C | GLN | D | 477 | 72.355 | 101.845 | 192.331 | 1.00 | 83.04 |
| 4502 | O | GLN | D | 477 | 73.095 | 101.333 | 191.489 | 1.00 | 85.92 |
| 4503 | N | TRP | D | 478 | 72.829 | 102.546 | 193.349 | 1.00 | 78.76 |
| 4504 | CA | TRP | D | 478 | 74.264 | 102.706 | 193.495 | 1.00 | 100.96 |
| 4505 | CB | TRP | D | 478 | 74.714 | 104.068 | 192.967 | 1.00 | 36.74 |
| 4506 | CG | TRP | D | 478 | 74.440 | 104.322 | 191.529 | 1.00 | 63.43 |
| 4507 | CD2 | TRP | D | 478 | 75.397 | 104.315 | 190.457 | 1.00 | 64.22 |
| 4508 | CE2 | TRP | D | 478 | 74.751 | 104.834 | 189.318 | 1.00 | 63.92 |
| 4509 | CE3 | TRP | D | 478 | 76.742 | 103.930 | 190.355 | 1.00 | 92.35 |
| 4510 | CD1 | TRP | D | 478 | 73.285 | 104.799 | 191.002 | 1.00 | 69.30 |
| 4511 | NE1 | TRP | D | 478 | 73.461 | 105.120 | 189.676 | 1.00 | 83.14 |
| 4512 | CZ2 | TRP | D | 478 | 75.402 | 104.985 | 188.088 | 1.00 | 106.36 |
| 4513 | CZ3 | TRP | D | 478 | 77.391 | 104.080 | 189.132 | 1.00 | 74.82 |
| 4514 | CH2 | TRP | D | 478 | 76.719 | 104.606 | 188.016 | 1.00 | 79.83 |
| 4515 | C | TRP | D | 478 | 74.723 | 102.569 | 194.938 | 1.00 | 79.39 |
| 4516 | O | TRP | D | 478 | 73.959 | 102.797 | 195.866 | 1.00 | 116.11 |
| 4517 | N | LEU | D | 479 | 75.982 | 102.200 | 195.113 | 1.00 | 66.99 |
| 4518 | CA | LEU | D | 479 | 76.560 | 102.055 | 196.428 | 1.00 | 70.32 |
| 4519 | CB | LEU | D | 479 | 76.867 | 100.593 | 196.694 | 1.00 | 83.07 |
| 4520 | CG | LEU | D | 479 | 75.686 | 99.688 | 196.347 | 1.00 | 60.88 |
| 4521 | CD1 | LEU | D | 479 | 76.079 | 98.229 | 196.566 | 1.00 | 143.46 |
| 4522 | CD2 | LEU | D | 479 | 74.497 | 100.046 | 197.202 | 1.00 | 93.52 |
| 4523 | C | LEU | D | 479 | 77.834 | 102.878 | 196.431 | 1.00 | 94.55 |
| 4524 | O | LEU | D | 479 | 78.361 | 103.209 | 195.371 | 1.00 | 87.16 |
| 4525 | N | HIS | D | 480 | 78.323 | 103.214 | 197.619 | 1.00 | 131.47 |
| 4526 | CA | HIS | D | 480 | 79.537 | 104.013 | 197.746 | 1.00 | 117.34 |
| 4527 | CB | HIS | D | 480 | 79.249 | 105.474 | 197.383 | 1.00 | 110.78 |
| 4528 | CG | HIS | D | 480 | 80.387 | 106.413 | 197.658 | 1.00 | 107.86 |
| 4529 | CD2 | HIS | D | 480 | 80.407 | 107.644 | 198.226 | 1.00 | 112.04 |
| 4530 | ND1 | HIS | D | 480 | 81.683 | 106.156 | 197.264 | 1.00 | 95.10 |
| 4531 | CE1 | HIS | D | 480 | 82.449 | 107.187 | 197.572 | 1.00 | 106.89 |
| 4532 | NE2 | HIS | D | 480 | 81.698 | 108.104 | 198.156 | 1.00 | 113.38 |
| 4533 | C | HIS | D | 480 | 80.068 | 103.931 | 199.157 | 1.00 | 92.28 |
| 4534 | O | HIS | D | 480 | 79.612 | 104.647 | 200.049 | 1.00 | 92.69 |
| 4535 | N | ASN | D | 481 | 81.028 | 103.043 | 199.362 | 1.00 | 97.73 |
| 4536 | CA | ASN | D | 481 | 81.609 | 102.894 | 200.677 | 1.00 | 121.93 |
| 4537 | CB | ASN | D | 481 | 82.125 | 104.254 | 201.160 | 1.00 | 108.49 |
| 4538 | CG | ASN | D | 481 | 83.258 | 104.126 | 202.145 | 1.00 | 147.00 |
| 4539 | OD1 | ASN | D | 481 | 83.876 | 105.120 | 202.522 | 1.00 | 154.08 |
| 4540 | ND2 | ASN | D | 481 | 83.542 | 102.897 | 202.570 | 1.00 | 166.29 |
| 4541 | C | ASN | D | 481 | 80.546 | 102.355 | 201.630 | 1.00 | 81.70 |
| 4542 | O | ASN | D | 481 | 80.382 | 102.854 | 202.746 | 1.00 | 102.35 |
| 4543 | N | GLU | D | 482 | 79.817 | 101.342 | 201.169 | 1.00 | 78.94 |
| 4544 | CA | GLU | D | 482 | 78.776 | 100.706 | 201.975 | 1.00 | 126.14 |
| 4545 | CB | GLU | D | 482 | 79.365 | 100.258 | 203.324 | 1.00 | 135.98 |
| 4546 | CG | GLU | D | 482 | 80.643 | 99.421 | 203.217 | 1.00 | 152.58 |
| 4547 | CD | GLU | D | 482 | 80.427 | 98.088 | 202.517 | 1.00 | 174.16 |
| 4548 | OE1 | GLU | D | 482 | 79.638 | 97.265 | 203.030 | 1.00 | 170.45 |
| 4549 | OE2 | GLU | D | 482 | 81.047 | 97.863 | 201.455 | 1.00 | 174.56 |
| 4550 | C | GLU | D | 482 | 77.575 | 101.627 | 202.215 | 1.00 | 109.93 |
| 4551 | O | GLU | D | 482 | 76.820 | 101.447 | 203.175 | 1.00 | 82.30 |
| 4552 | N | VAL | D | 483 | 77.392 | 102.605 | 201.336 | 1.00 | 76.74 |
| 4553 | CA | VAL | D | 483 | 76.287 | 103.551 | 201.477 | 1.00 | 102.62 |
| 4554 | CB | VAL | D | 483 | 76.815 | 104.983 | 201.744 | 1.00 | 109.65 |
| 4555 | CG1 | VAL | D | 483 | 75.669 | 105.986 | 201.713 | 1.00 | 98.68 |
| 4556 | CG2 | VAL | D | 483 | 77.519 | 105.028 | 203.088 | 1.00 | 170.08 |
| 4557 | C | VAL | D | 483 | 75.392 | 103.588 | 200.243 | 1.00 | 117.05 |
| 4558 | O | VAL | D | 483 | 75.779 | 104.119 | 199.202 | 1.00 | 143.84 |
| 4559 | N | GLN | D | 484 | 74.193 | 103.027 | 200.365 | 1.00 | 104.30 |
| 4560 | CA | GLN | D | 484 | 73.244 | 103.003 | 199.258 | 1.00 | 63.80 |
| 4561 | CB | GLN | D | 484 | 72.062 | 102.112 | 199.617 | 1.00 | 106.13 |
| 4562 | CG | GLN | D | 484 | 71.037 | 101.964 | 198.510 | 1.00 | 90.87 |
| 4563 | CD | GLN | D | 484 | 69.823 | 101.181 | 198.969 | 1.00 | 129.10 |
| 4564 | OE1 | GLN | D | 484 | 69.946 | 100.156 | 199.659 | 1.00 | 95.62 |
| 4565 | NE2 | GLN | D | 484 | 68.639 | 101.654 | 198.587 | 1.00 | 134.96 |
| 4566 | C | GLN | D | 484 | 72.750 | 104.413 | 198.969 | 1.00 | 52.20 |
| 4567 | O | GLN | D | 484 | 71.799 | 104.874 | 199.588 | 1.00 | 109.44 |
| 4568 | N | LEU | D | 485 | 73.377 | 105.098 | 198.024 | 1.00 | 55.15 |
| 4569 | CA | LEU | D | 485 | 72.991 | 106.471 | 197.711 | 1.00 | 71.69 |
| 4570 | CB | LEU | D | 485 | 73.698 | 106.961 | 196.447 | 1.00 | 66.01 |
| 4571 | CG | LEU | D | 485 | 75.216 | 107.146 | 196.541 | 1.00 | 75.22 |
| 4572 | CD1 | LEU | D | 485 | 75.661 | 108.084 | 195.431 | 1.00 | 81.39 |
| 4573 | CD2 | LEU | D | 485 | 75.604 | 107.739 | 197.885 | 1.00 | 111.54 |
| 4574 | C | LEU | D | 485 | 71.504 | 106.758 | 197.579 | 1.00 | 78.22 |
| 4575 | O | LEU | D | 485 | 70.698 | 105.854 | 197.350 | 1.00 | 76.73 |
| 4576 | N | PRO | D | 486 | 71.127 | 108.040 | 197.735 | 1.00 | 77.50 |
| 4577 | CD | PRO | D | 486 | 72.025 | 109.127 | 198.161 | 1.00 | 119.22 |
| 4578 | CA | PRO | D | 486 | 69.749 | 108.532 | 197.646 | 1.00 | 96.61 |
| 4579 | CB | PRO | D | 486 | 69.876 | 110.004 | 198.038 | 1.00 | 131.87 |
| 4580 | CG | PRO | D | 486 | 71.086 | 110.026 | 198.916 | 1.00 | 136.66 |
| 4581 | C | PRO | D | 486 | 69.179 | 108.380 | 196.244 | 1.00 | 92.91 |
| 4582 | O | PRO | D | 486 | 69.756 | 108.858 | 195.264 | 1.00 | 55.14 |
| 4583 | N | ASP | D | 487 | 68.027 | 107.731 | 196.163 | 1.00 | 82.83 |
| 4584 | CA | ASP | D | 487 | 67.380 | 107.504 | 194.888 | 1.00 | 93.17 |
| 4585 | CB | ASP | D | 487 | 65.947 | 107.016 | 195.124 | 1.00 | 115.19 |
| 4586 | CG | ASP | D | 487 | 65.437 | 106.130 | 193.996 | 1.00 | 161.24 |
| 4587 | OD1 | ASP | D | 487 | 65.248 | 106.640 | 192.869 | 1.00 | 164.95 |
| 4588 | OD2 | ASP | D | 487 | 65.232 | 104.918 | 194.235 | 1.00 | 171.27 |
| 4589 | C | ASP | D | 487 | 67.383 | 108.752 | 193.995 | 1.00 | 74.98 |
| 4590 | O | ASP | D | 487 | 67.617 | 108.660 | 192.791 | 1.00 | 95.57 |
| 4591 | N | ALA | D | 488 | 67.149 | 109.920 | 194.583 | 1.00 | 74.62 |
| 4592 | CA | ALA | D | 488 | 67.100 | 111.162 | 193.812 | 1.00 | 77.13 |
| 4593 | CB | ALA | D | 488 | 66.474 | 112.268 | 194.656 | 1.00 | 113.89 |
| 4594 | C | ALA | D | 488 | 68.455 | 111.622 | 193.277 | 1.00 | 89.67 |
| 4595 | O | ALA | D | 488 | 68.559 | 112.683 | 192.647 | 1.00 | 78.05 |
| 4596 | N | ARG | D | 489 | 69.489 | 110.822 | 193.520 | 1.00 | 47.18 |
| 4597 | CA | ARG | D | 489 | 70.837 | 111.159 | 193.065 | 1.00 | 81.70 |
| 4598 | CB | ARG | D | 489 | 71.856 | 110.474 | 193.974 | 1.00 | 98.18 |
| 4599 | CG | ARG | D | 489 | 72.159 | 111.254 | 195.231 | 1.00 | 119.10 |
| 4600 | CD | ARG | D | 489 | 73.301 | 112.214 | 194.975 | 1.00 | 108.85 |
| 4601 | NE | ARG | D | 489 | 74.572 | 111.660 | 195.432 | 1.00 | 107.43 |
| 4602 | CZ | ARG | D | 489 | 75.764 | 112.102 | 195.042 | 1.00 | 115.63 |
| 4603 | NH1 | ARG | D | 489 | 75.858 | 113.107 | 194.175 | 1.00 | 102.89 |
| 4604 | NH2 | ARG | D | 489 | 76.863 | 111.548 | 195.532 | 1.00 | 113.00 |
| 4605 | C | ARG | D | 489 | 71.146 | 110.806 | 191.606 | 1.00 | 93.41 |
| 4606 | O | ARG | D | 489 | 71.879 | 111.526 | 190.920 | 1.00 | 91.06 |
| 4607 | N | HIS | D | 490 | 70.579 | 109.699 | 191.138 | 1.00 | 91.66 |
| 4608 | CA | HIS | D | 490 | 70.810 | 109.230 | 189.784 | 1.00 | 79.10 |
| 4609 | CB | HIS | D | 490 | 71.330 | 107.809 | 189.831 | 1.00 | 61.88 |
| 4610 | CG | HIS | D | 490 | 70.361 | 106.847 | 190.429 | 1.00 | 50.09 |
| 4611 | CD2 | HIS | D | 490 | 69.154 | 106.414 | 189.998 | 1.00 | 71.95 |
| 4612 | ND1 | HIS | D | 490 | 70.600 | 106.198 | 191.622 | 1.00 | 80.99 |
| 4613 | CE1 | HIS | D | 490 | 69.582 | 105.403 | 191.899 | 1.00 | 81.13 |
| 4614 | NE2 | HIS | D | 490 | 68.691 | 105.516 | 190.929 | 1.00 | 120.64 |
| 4615 | C | HIS | D | 490 | 69.567 | 109.238 | 188.925 | 1.00 | 73.38 |
| 4616 | O | HIS | D | 490 | 68.455 | 109.193 | 189.431 | 1.00 | 83.57 |
| 4617 | N | SER | D | 491 | 69.773 | 109.267 | 187.616 | 1.00 | 106.32 |
| 4618 | CA | SER | D | 491 | 68.676 | 109.246 | 186.663 | 1.00 | 99.85 |
| 4619 | CB | SER | D | 491 | 68.783 | 110.424 | 185.696 | 1.00 | 80.79 |
| 4620 | OG | SER | D | 491 | 67.681 | 110.437 | 184.802 | 1.00 | 153.32 |
| 4621 | C | SER | D | 491 | 68.731 | 107.937 | 185.879 | 1.00 | 95.44 |
| 4622 | O | SER | D | 491 | 69.712 | 107.647 | 185.204 | 1.00 | 77.44 |
| 4623 | N | THR | D | 492 | 67.678 | 107.140 | 185.974 | 1.00 | 107.33 |
| 4624 | CA | THR | D | 492 | 67.638 | 105.879 | 185.254 | 1.00 | 88.96 |
| 4625 | CB | THR | D | 492 | 67.385 | 104.714 | 186.217 | 1.00 | 93.46 |
| 4626 | OG1 | THR | D | 492 | 68.540 | 104.532 | 187.044 | 1.00 | 95.38 |
| 4627 | CG2 | THR | D | 492 | 67.093 | 103.435 | 185.453 | 1.00 | 86.11 |
| 4628 | C | THR | D | 492 | 66.529 | 105.932 | 184.216 | 1.00 | 97.57 |
| 4629 | O | THR | D | 492 | 65.418 | 106.361 | 184.520 | 1.00 | 115.51 |
| 4630 | N | THR | D | 493 | 66.826 | 105.501 | 182.993 | 1.00 | 66.72 |
| 4631 | CA | THR | D | 493 | 65.820 | 105.527 | 181.936 | 1.00 | 75.38 |
| 4632 | CB | THR | D | 493 | 66.444 | 105.482 | 180.543 | 1.00 | 81.55 |
| 4633 | OG1 | THR | D | 493 | 66.925 | 104.157 | 180.285 | 1.00 | 66.75 |
| 4634 | CG2 | THR | D | 493 | 67.587 | 106.470 | 180.445 | 1.00 | 83.27 |
| 4635 | C | THR | D | 493 | 64.856 | 104.354 | 182.017 | 1.00 | 88.44 |
| 4636 | O | THR | D | 493 | 64.911 | 103.529 | 182.941 | 1.00 | 70.82 |
| 4637 | N | GLN | D | 494 | 63.964 | 104.296 | 181.036 | 1.00 | 95.57 |
| 4638 | CA | GLN | D | 494 | 62.988 | 103.229 | 180.976 | 1.00 | 86.74 |
| 4639 | CB | GLN | D | 494 | 61.641 | 103.773 | 180.491 | 1.00 | 104.79 |
| 4640 | CG | GLN | D | 494 | 61.002 | 104.821 | 181.399 | 1.00 | 121.61 |
| 4641 | CD | GLN | D | 494 | 60.761 | 104.317 | 182.821 | 1.00 | 154.81 |
| 4642 | OE1 | GLN | D | 494 | 60.297 | 103.194 | 183.027 | 1.00 | 127.19 |
| 4643 | NE2 | GLN | D | 494 | 61.064 | 105.158 | 183.806 | 1.00 | 163.95 |
| 4644 | C | GLN | D | 494 | 63.500 | 102.163 | 180.014 | 1.00 | 105.68 |
| 4645 | O | GLN | D | 494 | 64.174 | 102.473 | 179.029 | 1.00 | 117.51 |
| 4646 | N | PRO | D | 495 | 63.204 | 100.889 | 180.301 | 1.00 | 74.85 |
| 4647 | CD | PRO | D | 495 | 62.540 | 100.401 | 181.518 | 1.00 | 68.14 |
| 4648 | CA | PRO | D | 495 | 63.620 | 99.765 | 179.469 | 1.00 | 67.40 |
| 4649 | CB | PRO | D | 495 | 62.844 | 98.607 | 180.069 | 1.00 | 57.90 |
| 4650 | CG | PRO | D | 495 | 62.886 | 98.927 | 181.501 | 1.00 | 51.00 |
| 4651 | C | PRO | D | 495 | 63.295 | 99.968 | 177.996 | 1.00 | 77.06 |
| 4652 | O | PRO | D | 495 | 62.317 | 100.621 | 177.647 | 1.00 | 102.71 |
| 4653 | N | ARG | D | 496 | 64.136 | 99.410 | 177.138 | 1.00 | 110.77 |
| 4654 | CA | ARG | D | 496 | 63.956 | 99.496 | 175.697 | 1.00 | 122.60 |
| 4655 | CB | ARG | D | 496 | 64.765 | 100.668 | 175.128 | 1.00 | 143.51 |
| 4656 | CG | ARG | D | 496 | 64.080 | 102.027 | 175.280 | 1.00 | 154.62 |
| 4657 | CD | ARG | D | 496 | 65.003 | 103.174 | 174.882 | 1.00 | 172.80 |
| 4658 | NE | ARG | D | 496 | 64.275 | 104.427 | 174.686 | 1.00 | 206.30 |
| 4659 | CZ | ARG | D | 496 | 63.560 | 104.716 | 173.603 | 1.00 | 204.90 |
| 4660 | NH1 | ARG | D | 496 | 63.477 | 103.842 | 172.608 | 1.00 | 198.29 |
| 4661 | NH2 | ARG | D | 496 | 62.923 | 105.877 | 173.515 | 1.00 | 192.83 |
| 4662 | C | ARG | D | 496 | 64.426 | 98.170 | 175.119 | 1.00 | 131.58 |
| 4663 | O | ARG | D | 496 | 65.365 | 97.568 | 175.630 | 1.00 | 107.54 |
| 4664 | N | LYS | D | 497 | 63.767 | 97.707 | 174.063 | 1.00 | 139.06 |
| 4665 | CA | LYS | D | 497 | 64.114 | 96.427 | 173.459 | 1.00 | 98.97 |
| 4666 | CB | LYS | D | 497 | 62.936 | 95.900 | 172.636 | 1.00 | 111.63 |
| 4667 | CG | LYS | D | 497 | 61.645 | 95.750 | 173.428 | 1.00 | 148.53 |
| 4668 | CD | LYS | D | 497 | 60.515 | 95.187 | 172.575 | 1.00 | 162.48 |
| 4669 | CE | LYS | D | 497 | 59.221 | 95.078 | 173.374 | 1.00 | 159.87 |
| 4670 | NZ | LYS | D | 497 | 58.111 | 94.478 | 172.579 | 1.00 | 147.14 |
| 4671 | C | LYS | D | 497 | 65.360 | 96.466 | 172.594 | 1.00 | 102.21 |
| 4672 | O | LYS | D | 497 | 65.804 | 97.525 | 172.155 | 1.00 | 87.29 |
| 4673 | N | THR | D | 498 | 65.919 | 95.287 | 172.358 | 1.00 | 121.14 |
| 4674 | CA | THR | D | 498 | 67.108 | 95.144 | 171.536 | 1.00 | 136.22 |
| 4675 | CB | THR | D | 498 | 68.350 | 94.851 | 172.400 | 1.00 | 124.43 |
| 4676 | OG1 | THR | D | 498 | 68.079 | 93.769 | 173.302 | 1.00 | 102.03 |
| 4677 | CG2 | THR | D | 498 | 68.724 | 96.073 | 173.194 | 1.00 | 135.22 |
| 4678 | C | THR | D | 498 | 66.928 | 94.020 | 170.520 | 1.00 | 160.20 |
| 4679 | O | THR | D | 498 | 66.064 | 93.148 | 170.677 | 1.00 | 127.05 |
| 4680 | N | LYS | D | 499 | 67.746 | 94.045 | 169.473 | 1.00 | 148.81 |
| 4681 | CA | LYS | D | 499 | 67.665 | 93.030 | 168.436 | 1.00 | 167.95 |
| 4682 | CB | LYS | D | 499 | 68.518 | 93.442 | 167.227 | 1.00 | 183.82 |
| 4683 | CG | LYS | D | 499 | 68.240 | 92.635 | 165.953 | 1.00 | 194.67 |
| 4684 | CD | LYS | D | 499 | 68.915 | 93.247 | 164.723 | 1.00 | 178.66 |
| 4685 | CE | LYS | D | 499 | 68.530 | 92.509 | 163.439 | 1.00 | 166.21 |
| 4686 | NZ | LYS | D | 499 | 69.080 | 93.166 | 162.212 | 1.00 | 129.82 |
| 4687 | C | LYS | D | 499 | 68.131 | 91.685 | 168.993 | 1.00 | 174.78 |
| 4688 | O | LYS | D | 499 | 68.477 | 90.773 | 168.243 | 1.00 | 193.59 |
| 4689 | N | GLY | D | 500 | 68.132 | 91.570 | 170.318 | 1.00 | 172.34 |
| 4690 | CA | GLY | D | 500 | 68.549 | 90.339 | 170.963 | 1.00 | 167.96 |
| 4691 | C | GLY | D | 500 | 67.489 | 89.841 | 171.925 | 1.00 | 174.62 |
| 4692 | O | GLY | D | 500 | 67.796 | 89.121 | 172.875 | 1.00 | 180.27 |
| 4693 | N | SER | D | 501 | 66.244 | 90.244 | 171.674 | 1.00 | 149.85 |
| 4694 | CA | SER | D | 501 | 65.086 | 89.863 | 172.485 | 1.00 | 145.87 |
| 4695 | CB | SER | D | 501 | 64.828 | 88.355 | 172.379 | 1.00 | 150.97 |
| 4696 | OG | SER | D | 501 | 65.865 | 87.601 | 172.983 | 1.00 | 182.29 |
| 4697 | C | SER | D | 501 | 65.171 | 90.255 | 173.960 | 1.00 | 148.85 |
| 4698 | O | SER | D | 501 | 64.317 | 89.871 | 174.758 | 1.00 | 131.95 |
| 4699 | N | GLY | D | 502 | 66.194 | 91.018 | 174.322 | 1.00 | 141.68 |
| 4700 | CA | GLY | D | 502 | 66.334 | 91.439 | 175.703 | 1.00 | 99.20 |
| 4701 | C | GLY | D | 502 | 66.210 | 92.943 | 175.805 | 1.00 | 120.83 |
| 4702 | O | GLY | D | 502 | 66.192 | 93.629 | 174.781 | 1.00 | 107.61 |
| 4703 | N | PHE | D | 503 | 66.121 | 93.465 | 177.027 | 1.00 | 106.97 |
| 4704 | CA | PHE | D | 503 | 66.005 | 94.906 | 177.208 | 1.00 | 87.81 |
| 4705 | CB | PHE | D | 503 | 64.911 | 95.253 | 178.201 | 1.00 | 59.57 |
| 4706 | CG | PHE | D | 503 | 63.595 | 94.625 | 177.908 | 1.00 | 73.29 |
| 4707 | CD1 | PHE | D | 503 | 63.264 | 93.397 | 178.453 | 1.00 | 86.43 |
| 4708 | CD2 | PHE | D | 503 | 62.658 | 95.290 | 177.144 | 1.00 | 53.84 |
| 4709 | CE1 | PHE | D | 503 | 62.015 | 92.845 | 178.250 | 1.00 | 84.48 |
| 4710 | CE2 | PHE | D | 503 | 61.404 | 94.747 | 176.932 | 1.00 | 115.27 |
| 4711 | CZ | PHE | D | 503 | 61.081 | 93.520 | 177.490 | 1.00 | 109.78 |
| 4712 | C | PHE | D | 503 | 67.293 | 95.543 | 177.700 | 1.00 | 86.70 |
| 4713 | O | PHE | D | 503 | 68.236 | 94.851 | 178.074 | 1.00 | 68.11 |
| 4714 | N | PHE | D | 504 | 67.304 | 96.874 | 177.720 | 1.00 | 64.93 |
| 4715 | CA | PHE | D | 504 | 68.463 | 97.628 | 178.160 | 1.00 | 58.80 |
| 4716 | CB | PHE | D | 504 | 69.399 | 97.867 | 176.972 | 1.00 | 62.27 |
| 4717 | CG | PHE | D | 504 | 69.039 | 99.068 | 176.119 | 1.00 | 49.02 |
| 4718 | CD1 | PHE | D | 504 | 69.470 | 100.338 | 176.467 | 1.00 | 69.40 |
| 4719 | CD2 | PHE | D | 504 | 68.314 | 98.916 | 174.948 | 1.00 | 92.30 |
| 4720 | CE1 | PHE | D | 504 | 69.192 | 101.431 | 175.665 | 1.00 | 83.85 |
| 4721 | CE2 | PHE | D | 504 | 68.030 | 100.006 | 174.138 | 1.00 | 117.28 |
| 4722 | CZ | PHE | D | 504 | 68.474 | 101.266 | 174.498 | 1.00 | 115.08 |
| 4723 | C | PHE | D | 504 | 68.066 | 98.962 | 178.779 | 1.00 | 67.09 |
| 4724 | O | PHE | D | 504 | 67.236 | 99.690 | 178.233 | 1.00 | 100.63 |
| 4725 | N | VAL | D | 505 | 68.662 | 99.283 | 179.920 | 1.00 | 82.59 |
| 4726 | CA | VAL | D | 505 | 68.376 | 100.546 | 180.583 | 1.00 | 86.93 |
| 4727 | CB | VAL | D | 505 | 67.554 | 100.339 | 181.865 | 1.00 | 66.93 |
| 4728 | CG1 | VAL | D | 505 | 68.374 | 99.626 | 182.909 | 1.00 | 44.42 |
| 4729 | CG2 | VAL | D | 505 | 67.089 | 101.683 | 182.388 | 1.00 | 110.77 |
| 4730 | C | VAL | D | 505 | 69.672 | 101.266 | 180.936 | 1.00 | 63.33 |
| 4731 | O | VAL | D | 505 | 70.685 | 100.634 | 181.212 | 1.00 | 81.80 |
| 4732 | N | PHE | D | 506 | 69.629 | 102.592 | 180.924 | 1.00 | 82.05 |
| 4733 | CA | PHE | D | 506 | 70.796 | 103.414 | 181.227 | 1.00 | 74.33 |
| 4734 | CB | PHE | D | 506 | 71.026 | 104.405 | 180.096 | 1.00 | 64.40 |
| 4735 | CG | PHE | D | 506 | 71.958 | 103.925 | 179.028 | 1.00 | 65.82 |
| 4736 | CD1 | PHE | D | 506 | 71.821 | 104.396 | 177.728 | 1.00 | 93.15 |
| 4737 | CD2 | PHE | D | 506 | 73.015 | 103.082 | 179.321 | 1.00 | 97.38 |
| 4738 | CE1 | PHE | D | 506 | 72.715 | 104.046 | 176.741 | 1.00 | 60.40 |
| 4739 | CE2 | PHE | D | 506 | 73.920 | 102.723 | 178.335 | 1.00 | 94.39 |
| 4740 | CZ | PHE | D | 506 | 73.770 | 103.207 | 177.044 | 1.00 | 83.28 |
| 4741 | C | PHE | D | 506 | 70.632 | 104.203 | 182.525 | 1.00 | 84.85 |
| 4742 | O | PHE | D | 506 | 69.530 | 104.648 | 182.857 | 1.00 | 134.54 |
| 4743 | N | SER | D | 507 | 71.736 | 104.387 | 183.248 | 1.00 | 93.21 |
| 4744 | CA | SER | D | 507 | 71.722 | 105.144 | 184.500 | 1.00 | 85.59 |
| 4745 | CB | SER | D | 507 | 71.894 | 104.219 | 185.700 | 1.00 | 41.54 |
| 4746 | OG | SER | D | 507 | 71.868 | 104.985 | 186.887 | 1.00 | 68.49 |
| 4747 | C | SER | D | 507 | 72.833 | 106.187 | 184.516 | 1.00 | 67.47 |
| 4748 | O | SER | D | 507 | 73.925 | 105.945 | 184.012 | 1.00 | 59.51 |
| 4749 | N | ARG | D | 508 | 72.550 | 107.342 | 185.105 | 1.00 | 61.04 |
| 4750 | CA | ARG | D | 508 | 73.517 | 108.428 | 185.175 | 1.00 | 58.84 |
| 4751 | CB | ARG | D | 508 | 73.139 | 109.517 | 184.167 | 1.00 | 49.25 |
| 4752 | CG | ARG | D | 508 | 73.977 | 110.781 | 184.239 | 1.00 | 60.80 |
| 4753 | CD | ARG | D | 508 | 73.595 | 111.707 | 183.087 | 1.00 | 74.40 |
| 4754 | NE | ARG | D | 508 | 74.244 | 113.013 | 183.150 | 1.00 | 57.97 |
| 4755 | CZ | ARG | D | 508 | 73.940 | 113.952 | 184.042 | 1.00 | 108.28 |
| 4756 | NH1 | ARG | D | 508 | 72.994 | 113.730 | 184.947 | 1.00 | 134.73 |
| 4757 | NH2 | ARG | D | 508 | 74.574 | 115.118 | 184.028 | 1.00 | 134.59 |
| 4758 | C | ARG | D | 508 | 73.579 | 108.999 | 186.586 | 1.00 | 66.73 |
| 4759 | O | ARG | D | 508 | 72.559 | 109.381 | 187.162 | 1.00 | 94.10 |
| 4760 | N | LEU | D | 509 | 74.792 | 109.076 | 187.123 | 1.00 | 102.57 |
| 4761 | CA | LEU | D | 509 | 75.009 | 109.556 | 188.479 | 1.00 | 92.49 |
| 4762 | CB | LEU | D | 509 | 75.349 | 108.353 | 189.361 | 1.00 | 65.32 |
| 4763 | CG | LEU | D | 509 | 75.820 | 108.672 | 190.775 | 1.00 | 82.75 |
| 4764 | CD1 | LEU | D | 509 | 74.814 | 109.609 | 191.434 | 1.00 | 121.62 |
| 4765 | CD2 | LEU | D | 509 | 75.980 | 107.383 | 191.571 | 1.00 | 45.98 |
| 4766 | C | LEU | D | 509 | 76.108 | 110.609 | 188.620 | 1.00 | 66.30 |
| 4767 | O | LEU | D | 509 | 77.252 | 110.253 | 188.843 | 1.00 | 62.93 |
| 4768 | N | GLU | D | 510 | 75.774 | 111.893 | 188.504 | 1.00 | 101.58 |
| 4769 | CA | GLU | D | 510 | 76.793 | 112.943 | 188.641 | 1.00 | 93.28 |
| 4770 | CB | GLU | D | 510 | 76.143 | 114.333 | 188.575 | 1.00 | 108.16 |
| 4771 | CG | GLU | D | 510 | 75.472 | 114.649 | 187.240 | 1.00 | 127.89 |
| 4772 | CD | GLU | D | 510 | 74.727 | 115.982 | 187.233 | 1.00 | 158.27 |
| 4773 | OE1 | GLU | D | 510 | 73.731 | 116.114 | 187.978 | 1.00 | 163.67 |
| 4774 | OE2 | GLU | D | 510 | 75.133 | 116.898 | 186.481 | 1.00 | 133.07 |
| 4775 | C | GLU | D | 510 | 77.516 | 112.757 | 189.981 | 1.00 | 107.52 |
| 4776 | O | GLU | D | 510 | 76.906 | 112.303 | 190.949 | 1.00 | 106.06 |
| 4777 | N | VAL | D | 511 | 78.808 | 113.095 | 190.036 | 1.00 | 92.80 |
| 4778 | CA | VAL | D | 511 | 79.599 | 112.937 | 191.268 | 1.00 | 119.85 |
| 4779 | CB | VAL | D | 511 | 80.495 | 111.658 | 191.193 | 1.00 | 42.93 |
| 4780 | CG1 | VAL | D | 511 | 81.422 | 111.579 | 192.387 | 1.00 | 117.40 |
| 4781 | CG2 | VAL | D | 511 | 79.622 | 110.425 | 191.171 | 1.00 | 107.86 |
| 4782 | C | VAL | D | 511 | 80.483 | 114.145 | 191.640 | 1.00 | 159.37 |
| 4783 | O | VAL | D | 511 | 80.869 | 114.943 | 190.776 | 1.00 | 157.18 |
| 4784 | N | THR | D | 512 | 80.795 | 114.261 | 192.936 | 1.00 | 172.76 |
| 4785 | CA | THR | D | 512 | 81.622 | 115.346 | 193.472 | 1.00 | 158.22 |
| 4786 | CB | THR | D | 512 | 80.946 | 116.001 | 194.700 | 1.00 | 168.13 |
| 4787 | OG1 | THR | D | 512 | 79.636 | 116.460 | 194.340 | 1.00 | 167.64 |
| 4788 | CG2 | THR | D | 512 | 81.773 | 117.182 | 195.203 | 1.00 | 185.75 |
| 4789 | C | THR | D | 512 | 83.020 | 114.866 | 193.889 | 1.00 | 141.73 |
| 4790 | O | THR | D | 512 | 83.171 | 113.781 | 194.468 | 1.00 | 95.92 |
| 4791 | N | ARG | D | 513 | 84.029 | 115.691 | 193.598 | 1.00 | 133.96 |
| 4792 | CA | ARG | D | 513 | 85.423 | 115.383 | 193.919 | 1.00 | 137.46 |
| 4793 | CB | ARG | D | 513 | 86.296 | 116.640 | 193.794 | 1.00 | 162.09 |
| 4794 | CG | ARG | D | 513 | 87.780 | 116.393 | 194.082 | 1.00 | 185.62 |
| 4795 | CD | ARG | D | 513 | 88.601 | 117.679 | 194.049 | 1.00 | 208.34 |
| 4796 | NE | ARG | D | 513 | 90.013 | 117.433 | 194.340 | 1.00 | 232.40 |
| 4797 | CZ | ARG | D | 513 | 90.943 | 118.383 | 194.427 | 1.00 | 235.91 |
| 4798 | NH1 | ARG | D | 513 | 90.620 | 119.656 | 194.244 | 1.00 | 230.77 |
| 4799 | NH2 | ARG | D | 513 | 92.201 | 118.059 | 194.699 | 1.00 | 224.77 |
| 4800 | C | ARG | D | 513 | 85.580 | 114.803 | 195.315 | 1.00 | 122.87 |
| 4801 | O | ARG | D | 513 | 86.170 | 113.737 | 195.499 | 1.00 | 77.13 |
| 4802 | N | ALA | D | 514 | 85.050 | 115.520 | 196.295 | 1.00 | 123.66 |
| 4803 | CA | ALA | D | 514 | 85.120 | 115.095 | 197.682 | 1.00 | 119.89 |
| 4804 | CB | ALA | D | 514 | 84.105 | 115.872 | 198.496 | 1.00 | 121.33 |
| 4805 | C | ALA | D | 514 | 84.881 | 113.594 | 197.847 | 1.00 | 114.46 |
| 4806 | O | ALA | D | 514 | 85.618 | 112.909 | 198.559 | 1.00 | 115.15 |
| 4807 | N | GLU | D | 515 | 83.860 | 113.086 | 197.165 | 1.00 | 114.63 |
| 4808 | CA | GLU | D | 515 | 83.502 | 111.677 | 197.265 | 1.00 | 110.01 |
| 4809 | CB | GLU | D | 515 | 82.120 | 111.454 | 196.648 | 1.00 | 77.08 |
| 4810 | CG | GLU | D | 515 | 81.013 | 112.106 | 197.450 | 1.00 | 144.14 |
| 4811 | CD | GLU | D | 515 | 79.656 | 111.961 | 196.806 | 1.00 | 162.03 |
| 4812 | OE1 | GLU | D | 515 | 79.475 | 112.477 | 195.680 | 1.00 | 133.35 |
| 4813 | OE2 | GLU | D | 515 | 78.773 | 111.334 | 197.431 | 1.00 | 167.09 |
| 4814 | C | GLU | D | 515 | 84.480 | 110.641 | 196.718 | 1.00 | 110.50 |
| 4815 | O | GLU | D | 515 | 84.593 | 109.548 | 197.282 | 1.00 | 87.75 |
| 4816 | N | TRP | D | 516 | 85.188 | 110.948 | 195.635 | 1.00 | 88.87 |
| 4817 | CA | TRP | D | 516 | 86.111 | 109.943 | 195.117 | 1.00 | 96.30 |
| 4818 | CB | TRP | D | 516 | 86.285 | 110.065 | 193.596 | 1.00 | 116.50 |
| 4819 | CG | TRP | D | 516 | 87.195 | 111.132 | 193.088 | 1.00 | 89.33 |
| 4820 | CD2 | TRP | D | 516 | 86.815 | 112.276 | 192.315 | 1.00 | 84.44 |
| 4821 | CE2 | TRP | D | 516 | 88.002 | 112.945 | 191.949 | 1.00 | 97.81 |
| 4822 | CE3 | TRP | D | 516 | 85.585 | 112.796 | 191.892 | 1.00 | 94.14 |
| 4823 | CD1 | TRP | D | 516 | 88.556 | 111.159 | 193.168 | 1.00 | 124.94 |
| 4824 | NE1 | TRP | D | 516 | 89.050 | 112.243 | 192.483 | 1.00 | 140.17 |
| 4825 | CZ2 | TRP | D | 516 | 87.996 | 114.115 | 191.178 | 1.00 | 128.17 |
| 4826 | CZ3 | TRP | D | 516 | 85.578 | 113.960 | 191.125 | 1.00 | 81.83 |
| 4827 | CH2 | TRP | D | 516 | 86.779 | 114.604 | 190.775 | 1.00 | 116.38 |
| 4828 | C | TRP | D | 516 | 87.452 | 109.954 | 195.824 | 1.00 | 127.96 |
| 4829 | O | TRP | D | 516 | 88.268 | 109.049 | 195.640 | 1.00 | 148.49 |
| 4830 | N | GLU | D | 517 | 87.676 | 110.982 | 196.636 | 1.00 | 129.08 |
| 4831 | CA | GLU | D | 517 | 88.906 | 111.068 | 197.400 | 1.00 | 111.06 |
| 4832 | CB | GLU | D | 517 | 89.239 | 112.523 | 197.722 | 1.00 | 135.30 |
| 4833 | CG | GLU | D | 517 | 89.577 | 113.349 | 196.490 | 1.00 | 155.78 |
| 4834 | CD | GLU | D | 517 | 90.194 | 114.688 | 196.834 | 1.00 | 196.31 |
| 4835 | OE1 | GLU | D | 517 | 89.548 | 115.475 | 197.559 | 1.00 | 211.98 |
| 4836 | OE2 | GLU | D | 517 | 91.328 | 114.952 | 196.380 | 1.00 | 180.61 |
| 4837 | C | GLU | D | 517 | 88.632 | 110.271 | 198.668 | 1.00 | 124.76 |
| 4838 | O | GLU | D | 517 | 89.549 | 109.710 | 199.278 | 1.00 | 141.89 |
| 4839 | N | GLN | D | 518 | 87.352 | 110.217 | 199.041 | 1.00 | 103.46 |
| 4840 | CA | GLN | D | 518 | 86.906 | 109.467 | 200.211 | 1.00 | 115.30 |
| 4841 | CB | GLN | D | 518 | 85.419 | 109.731 | 200.463 | 1.00 | 143.81 |
| 4842 | CG | GLN | D | 518 | 84.866 | 109.115 | 201.744 | 1.00 | 176.95 |
| 4843 | CD | GLN | D | 518 | 83.365 | 109.327 | 201.897 | 1.00 | 168.05 |
| 4844 | OE1 | GLN | D | 518 | 82.879 | 110.461 | 201.875 | 1.00 | 121.65 |
| 4845 | NE2 | GLN | D | 518 | 82.624 | 108.233 | 202.053 | 1.00 | 146.22 |
| 4846 | C | GLN | D | 518 | 87.136 | 107.988 | 199.890 | 1.00 | 126.60 |
| 4847 | O | GLN | D | 518 | 87.433 | 107.182 | 200.778 | 1.00 | 97.98 |
| 4848 | N | LYS | D | 519 | 86.987 | 107.660 | 198.605 | 1.00 | 133.48 |
| 4849 | CA | LYS | D | 519 | 87.195 | 106.314 | 198.065 | 1.00 | 130.66 |
| 4850 | CB | LYS | D | 519 | 86.243 | 105.295 | 198.698 | 1.00 | 75.97 |
| 4851 | CG | LYS | D | 519 | 86.644 | 103.852 | 198.402 | 1.00 | 89.89 |
| 4852 | CD | LYS | D | 519 | 86.097 | 102.873 | 199.431 | 1.00 | 118.90 |
| 4853 | CE | LYS | D | 519 | 86.781 | 101.513 | 199.312 | 1.00 | 120.25 |
| 4854 | NZ | LYS | D | 519 | 86.331 | 100.555 | 200.365 | 1.00 | 142.07 |
| 4855 | C | LYS | D | 519 | 86.960 | 106.353 | 196.560 | 1.00 | 122.50 |
| 4856 | O | LYS | D | 519 | 86.313 | 107.271 | 196.063 | 1.00 | 112.56 |
| 4857 | N | ASP | D | 520 | 87.494 | 105.364 | 195.840 | 1.00 | 147.72 |
| 4858 | CA | ASP | D | 520 | 87.334 | 105.277 | 194.383 | 1.00 | 101.05 |
| 4859 | CB | ASP | D | 520 | 88.642 | 104.843 | 193.718 | 1.00 | 139.44 |
| 4860 | CG | ASP | D | 520 | 89.593 | 105.997 | 193.499 | 1.00 | 177.33 |
| 4861 | OD1 | ASP | D | 520 | 89.228 | 106.917 | 192.737 | 1.00 | 157.39 |
| 4862 | OD2 | ASP | D | 520 | 90.698 | 105.982 | 194.086 | 1.00 | 181.89 |
| 4863 | C | ASP | D | 520 | 86.240 | 104.292 | 193.999 | 1.00 | 90.40 |
| 4864 | O | ASP | D | 520 | 85.540 | 104.494 | 193.015 | 1.00 | 100.30 |
| 4865 | N | GLU | D | 521 | 86.104 | 103.225 | 194.783 | 1.00 | 113.97 |
| 4866 | CA | GLU | D | 521 | 85.096 | 102.196 | 194.535 | 1.00 | 100.63 |
| 4867 | CB | GLU | D | 521 | 85.165 | 101.093 | 195.599 | 1.00 | 137.88 |
| 4868 | CG | GLU | D | 521 | 85.922 | 99.836 | 195.206 | 1.00 | 152.59 |
| 4869 | CD | GLU | D | 521 | 85.646 | 98.680 | 196.162 | 1.00 | 162.69 |
| 4870 | OE1 | GLU | D | 521 | 84.480 | 98.228 | 196.228 | 1.00 | 124.80 |
| 4871 | OE2 | GLU | D | 521 | 86.587 | 98.228 | 196.850 | 1.00 | 167.89 |
| 4872 | C | GLU | D | 521 | 83.661 | 102.715 | 194.504 | 1.00 | 98.08 |
| 4873 | O | GLU | D | 521 | 83.099 | 103.086 | 195.534 | 1.00 | 104.25 |
| 4874 | N | PHE | D | 522 | 83.078 | 102.726 | 193.312 | 1.00 | 127.92 |
| 4875 | CA | PHE | D | 522 | 81.692 | 103.137 | 193.112 | 1.00 | 85.49 |
| 4876 | CB | PHE | D | 522 | 81.596 | 104.278 | 192.104 | 1.00 | 55.87 |
| 4877 | CG | PHE | D | 522 | 81.809 | 105.627 | 192.700 | 1.00 | 92.01 |
| 4878 | CD1 | PHE | D | 522 | 82.876 | 105.862 | 193.553 | 1.00 | 109.83 |
| 4879 | CD2 | PHE | D | 522 | 80.950 | 106.672 | 192.392 | 1.00 | 125.42 |
| 4880 | CE1 | PHE | D | 522 | 83.080 | 107.123 | 194.087 | 1.00 | 147.56 |
| 4881 | CE2 | PHE | D | 522 | 81.146 | 107.935 | 192.919 | 1.00 | 81.53 |
| 4882 | CZ | PHE | D | 522 | 82.208 | 108.163 | 193.765 | 1.00 | 99.48 |
| 4883 | C | PHE | D | 522 | 80.989 | 101.915 | 192.556 | 1.00 | 91.67 |
| 4884 | O | PHE | D | 522 | 81.450 | 101.315 | 191.587 | 1.00 | 86.92 |
| 4885 | N | ILE | D | 523 | 79.879 | 101.532 | 193.159 | 1.00 | 58.82 |
| 4886 | CA | ILE | D | 523 | 79.199 | 100.360 | 192.670 | 1.00 | 96.62 |
| 4887 | CB | ILE | D | 523 | 79.059 | 99.315 | 193.791 | 1.00 | 67.53 |
| 4888 | CG2 | ILE | D | 523 | 78.386 | 98.043 | 193.261 | 1.00 | 71.95 |
| 4889 | CG1 | ILE | D | 523 | 80.448 | 98.994 | 194.353 | 1.00 | 74.60 |
| 4890 | CD1 | ILE | D | 523 | 80.448 | 97.914 | 195.426 | 1.00 | 142.45 |
| 4891 | C | ILE | D | 523 | 77.839 | 100.700 | 192.098 | 1.00 | 86.74 |
| 4892 | O | ILE | D | 523 | 77.110 | 101.506 | 192.654 | 1.00 | 100.77 |
| 4893 | N | CYS | D | 524 | 77.521 | 100.095 | 190.961 | 1.00 | 98.48 |
| 4894 | CA | CYS | D | 524 | 76.236 | 100.283 | 190.304 | 1.00 | 85.44 |
| 4895 | C | CYS | D | 524 | 75.553 | 98.922 | 190.326 | 1.00 | 64.47 |
| 4896 | O | CYS | D | 524 | 75.712 | 98.128 | 189.406 | 1.00 | 68.44 |
| 4897 | CB | CYS | D | 524 | 76.425 | 100.739 | 188.861 | 1.00 | 71.53 |
| 4898 | SG | CYS | D | 524 | 74.938 | 100.516 | 187.836 | 1.00 | 110.38 |
| 4899 | N | ARG | D | 525 | 74.809 | 98.655 | 191.395 | 1.00 | 92.59 |
| 4900 | CA | ARG | D | 525 | 74.107 | 97.387 | 191.569 | 1.00 | 59.38 |
| 4901 | CB | ARG | D | 525 | 73.679 | 97.234 | 193.029 | 1.00 | 53.65 |
| 4902 | CG | ARG | D | 525 | 73.283 | 95.825 | 193.416 | 1.00 | 62.42 |
| 4903 | CD | ARG | D | 525 | 73.547 | 95.533 | 194.901 | 1.00 | 81.91 |
| 4904 | NE | ARG | D | 525 | 72.599 | 96.177 | 195.808 | 1.00 | 90.24 |
| 4905 | CZ | ARG | D | 525 | 72.458 | 95.846 | 197.088 | 1.00 | 147.65 |
| 4906 | NH1 | ARG | D | 525 | 73.206 | 94.878 | 197.608 | 1.00 | 136.10 |
| 4907 | NH2 | ARG | D | 525 | 71.568 | 96.475 | 197.847 | 1.00 | 130.51 |
| 4908 | C | ARG | D | 525 | 72.890 | 97.286 | 190.665 | 1.00 | 81.55 |
| 4909 | O | ARG | D | 525 | 72.477 | 98.264 | 190.048 | 1.00 | 81.39 |
| 4910 | N | ALA | D | 526 | 72.325 | 96.089 | 190.590 | 1.00 | 78.78 |
| 4911 | CA | ALA | D | 526 | 71.152 | 95.837 | 189.770 | 1.00 | 57.14 |
| 4912 | CB | ALA | D | 526 | 71.564 | 95.534 | 188.356 | 1.00 | 39.11 |
| 4913 | C | ALA | D | 526 | 70.393 | 94.661 | 190.359 | 1.00 | 60.25 |
| 4914 | O | ALA | D | 526 | 70.982 | 93.665 | 190.776 | 1.00 | 67.91 |
| 4915 | N | VAL | D | 527 | 69.078 | 94.783 | 190.403 | 1.00 | 45.78 |
| 4916 | CA | VAL | D | 527 | 68.265 | 93.721 | 190.958 | 1.00 | 62.42 |
| 4917 | CB | VAL | D | 527 | 67.445 | 94.234 | 192.147 | 1.00 | 81.54 |
| 4918 | CG1 | VAL | D | 527 | 66.648 | 93.095 | 192.766 | 1.00 | 72.43 |
| 4919 | CG2 | VAL | D | 527 | 68.373 | 94.871 | 193.163 | 1.00 | 71.57 |
| 4920 | C | VAL | D | 527 | 67.326 | 93.199 | 189.894 | 1.00 | 54.64 |
| 4921 | O | VAL | D | 527 | 66.520 | 93.949 | 189.356 | 1.00 | 97.00 |
| 4922 | N | HIS | D | 528 | 67.443 | 91.914 | 189.584 | 1.00 | 64.63 |
| 4923 | CA | HIS | D | 528 | 66.588 | 91.301 | 188.581 | 1.00 | 51.86 |
| 4924 | CB | HIS | D | 528 | 67.337 | 91.112 | 187.275 | 1.00 | 37.99 |
| 4925 | CG | HIS | D | 528 | 66.459 | 90.762 | 186.118 | 1.00 | 60.26 |
| 4926 | CD2 | HIS | D | 528 | 65.631 | 89.713 | 185.908 | 1.00 | 94.26 |
| 4927 | ND1 | HIS | D | 528 | 66.402 | 91.528 | 184.973 | 1.00 | 110.60 |
| 4928 | CE1 | HIS | D | 528 | 65.580 | 90.963 | 184.107 | 1.00 | 110.22 |
| 4929 | NE2 | HIS | D | 528 | 65.099 | 89.860 | 184.651 | 1.00 | 125.81 |
| 4930 | C | HIS | D | 528 | 66.106 | 89.963 | 189.080 | 1.00 | 60.71 |
| 4931 | O | HIS | D | 528 | 66.741 | 89.363 | 189.953 | 1.00 | 64.08 |
| 4932 | N | GLU | D | 529 | 64.979 | 89.512 | 188.533 | 1.00 | 75.37 |
| 4933 | CA | GLU | D | 529 | 64.388 | 88.250 | 188.933 | 1.00 | 104.01 |
| 4934 | CB | GLU | D | 529 | 63.007 | 88.110 | 188.318 | 1.00 | 133.95 |
| 4935 | CG | GLU | D | 529 | 62.316 | 86.852 | 188.709 | 1.00 | 183.70 |
| 4936 | CD | GLU | D | 529 | 60.964 | 86.726 | 188.018 | 1.00 | 202.90 |
| 4937 | OE1 | GLU | D | 529 | 60.677 | 87.124 | 186.972 | 1.00 | 204.87 |
| 4938 | OE2 | GLU | D | 529 | 59.987 | 86.193 | 188.379 | 1.00 | 194.88 |
| 4939 | C | GLU | D | 529 | 65.266 | 87.069 | 188.541 | 1.00 | 117.67 |
| 4940 | O | GLU | D | 529 | 65.146 | 85.986 | 189.117 | 1.00 | 105.24 |
| 4941 | N | ALA | D | 530 | 66.198 | 87.305 | 187.616 | 1.00 | 104.62 |
| 4942 | CA | ALA | D | 530 | 67.094 | 86.252 | 187.128 | 1.00 | 119.79 |
| 4943 | CB | ALA | D | 530 | 67.429 | 86.491 | 185.653 | 1.00 | 110.35 |
| 4944 | C | ALA | D | 530 | 68.381 | 86.118 | 187.923 | 1.00 | 142.01 |
| 4945 | O | ALA | D | 530 | 68.445 | 85.370 | 188.898 | 1.00 | 165.83 |
| 4946 | N | ALA | D | 531 | 69.398 | 86.844 | 187.473 | 1.00 | 133.84 |
| 4947 | CA | ALA | D | 531 | 70.724 | 86.868 | 188.083 | 1.00 | 167.12 |
| 4948 | CB | ALA | D | 531 | 71.135 | 88.308 | 188.325 | 1.00 | 130.57 |
| 4949 | C | ALA | D | 531 | 70.905 | 86.066 | 189.370 | 1.00 | 184.40 |
| 4950 | O | ALA | D | 531 | 70.099 | 86.152 | 190.298 | 1.00 | 180.48 |
| 4951 | N | SER | D | 532 | 71.985 | 85.294 | 189.421 | 1.00 | 188.26 |
| 4952 | CA | SER | D | 532 | 72.294 | 84.494 | 190.596 | 1.00 | 165.83 |
| 4953 | CB | SER | D | 532 | 72.587 | 83.041 | 190.197 | 1.00 | 154.95 |
| 4954 | OG | SER | D | 532 | 71.402 | 82.364 | 189.812 | 1.00 | 130.00 |
| 4955 | C | SER | D | 532 | 73.494 | 85.093 | 191.329 | 1.00 | 145.61 |
| 4956 | O | SER | D | 532 | 74.236 | 85.909 | 190.775 | 1.00 | 131.77 |
| 4957 | N | PRO | D | 533 | 73.700 | 84.687 | 192.588 | 1.00 | 134.93 |
| 4958 | CD | PRO | D | 533 | 74.977 | 84.898 | 193.295 | 1.00 | 147.80 |
| 4959 | CA | PRO | D | 533 | 72.863 | 83.723 | 193.308 | 1.00 | 118.05 |
| 4960 | CB | PRO | D | 533 | 73.891 | 82.890 | 194.044 | 1.00 | 151.25 |
| 4961 | CG | PRO | D | 533 | 74.856 | 83.964 | 194.501 | 1.00 | 159.03 |
| 4962 | C | PRO | D | 533 | 71.913 | 84.426 | 194.272 | 1.00 | 133.53 |
| 4963 | O | PRO | D | 533 | 70.971 | 83.824 | 194.796 | 1.00 | 89.86 |
| 4964 | N | SER | D | 534 | 72.180 | 85.709 | 194.496 | 1.00 | 145.54 |
| 4965 | CA | SER | D | 534 | 71.391 | 86.530 | 195.406 | 1.00 | 131.75 |
| 4966 | CB | SER | D | 534 | 72.330 | 87.327 | 196.311 | 1.00 | 161.36 |
| 4967 | OG | SER | D | 534 | 73.274 | 88.055 | 195.538 | 1.00 | 169.84 |
| 4968 | C | SER | D | 534 | 70.454 | 87.488 | 194.679 | 1.00 | 120.79 |
| 4969 | O | SER | D | 534 | 70.006 | 88.477 | 195.250 | 1.00 | 109.78 |
| 4970 | N | GLN | D | 535 | 70.162 | 87.192 | 193.419 | 1.00 | 127.46 |
| 4971 | CA | GLN | D | 535 | 69.280 | 88.032 | 192.618 | 1.00 | 106.83 |
| 4972 | CB | GLN | D | 535 | 67.887 | 88.057 | 193.244 | 1.00 | 30.80 |
| 4973 | CG | GLN | D | 535 | 67.263 | 86.683 | 193.338 | 1.00 | 63.61 |
| 4974 | CD | GLN | D | 535 | 66.841 | 86.320 | 194.746 | 1.00 | 103.06 |
| 4975 | OE1 | GLN | D | 535 | 67.579 | 86.550 | 195.703 | 1.00 | 120.06 |
| 4976 | NE2 | GLN | D | 535 | 65.654 | 85.735 | 194.881 | 1.00 | 119.16 |
| 4977 | C | GLN | D | 535 | 69.834 | 89.450 | 192.454 | 1.00 | 81.50 |
| 4978 | O | GLN | D | 535 | 69.127 | 90.369 | 192.039 | 1.00 | 62.58 |
| 4979 | N | THR | D | 536 | 71.117 | 89.603 | 192.769 | 1.00 | 77.55 |
| 4980 | CA | THR | D | 536 | 71.818 | 90.876 | 192.651 | 1.00 | 78.91 |
| 4981 | CB | THR | D | 536 | 72.502 | 91.241 | 193.964 | 1.00 | 101.54 |
| 4982 | OG1 | THR | D | 536 | 71.513 | 91.416 | 194.980 | 1.00 | 139.48 |
| 4983 | CG2 | THR | D | 536 | 73.313 | 92.511 | 193.812 | 1.00 | 91.85 |
| 4984 | C | THR | D | 536 | 72.908 | 90.769 | 191.583 | 1.00 | 88.04 |
| 4985 | O | THR | D | 536 | 73.332 | 89.672 | 191.225 | 1.00 | 115.17 |
| 4986 | N | VAL | D | 537 | 73.368 | 91.914 | 191.092 | 1.00 | 73.07 |
| 4987 | CA | VAL | D | 537 | 74.415 | 91.965 | 190.078 | 1.00 | 83.98 |
| 4988 | CB | VAL | D | 537 | 73.851 | 91.681 | 188.687 | 1.00 | 56.48 |
| 4989 | CG1 | VAL | D | 537 | 74.764 | 92.252 | 187.629 | 1.00 | 76.12 |
| 4990 | CG2 | VAL | D | 537 | 73.701 | 90.191 | 188.490 | 1.00 | 116.74 |
| 4991 | C | VAL | D | 537 | 75.041 | 93.348 | 190.075 | 1.00 | 97.13 |
| 4992 | O | VAL | D | 537 | 74.349 | 94.346 | 189.894 | 1.00 | 104.54 |
| 4993 | N | GLN | D | 538 | 76.350 | 93.415 | 190.263 | 1.00 | 95.87 |
| 4994 | CA | GLN | D | 538 | 77.004 | 94.713 | 190.296 | 1.00 | 90.43 |
| 4995 | CB | GLN | D | 538 | 77.204 | 95.139 | 191.756 | 1.00 | 92.33 |
| 4996 | CG | GLN | D | 538 | 77.904 | 94.094 | 192.622 | 1.00 | 78.35 |
| 4997 | CD | GLN | D | 538 | 77.775 | 94.382 | 194.105 | 1.00 | 103.51 |
| 4998 | OE1 | GLN | D | 538 | 76.736 | 94.108 | 194.715 | 1.00 | 86.43 |
| 4999 | NE2 | GLN | D | 538 | 78.829 | 94.950 | 194.695 | 1.00 | 114.86 |
| 5000 | C | GLN | D | 538 | 78.329 | 94.733 | 189.554 | 1.00 | 78.31 |
| 5001 | O | GLN | D | 538 | 78.903 | 93.684 | 189.271 | 1.00 | 92.93 |
| 5002 | N | ARG | D | 539 | 78.794 | 95.936 | 189.229 | 1.00 | 52.71 |
| 5003 | CA | ARG | D | 539 | 80.060 | 96.120 | 188.539 | 1.00 | 93.72 |
| 5004 | CB | ARG | D | 539 | 79.846 | 96.340 | 187.042 | 1.00 | 98.94 |
| 5005 | CG | ARG | D | 539 | 81.128 | 96.234 | 186.207 | 1.00 | 143.16 |
| 5006 | CD | ARG | D | 539 | 81.220 | 94.892 | 185.479 | 1.00 | 155.59 |
| 5007 | NE | ARG | D | 539 | 80.795 | 93.779 | 186.324 | 1.00 | 160.22 |
| 5008 | CZ | ARG | D | 539 | 80.719 | 92.516 | 185.921 | 1.00 | 144.73 |
| 5009 | NH1 | ARG | D | 539 | 81.046 | 92.195 | 184.678 | 1.00 | 164.97 |
| 5010 | NH2 | ARG | D | 539 | 80.302 | 91.578 | 186.760 | 1.00 | 105.83 |
| 5011 | C | ARG | D | 539 | 80.713 | 97.352 | 189.131 | 1.00 | 116.29 |
| 5012 | O | ARG | D | 539 | 80.119 | 98.424 | 189.130 | 1.00 | 77.60 |
| 5013 | N | ALA | D | 540 | 81.933 | 97.195 | 189.634 | 1.00 | 123.66 |
| 5014 | CA | ALA | D | 540 | 82.664 | 98.303 | 190.232 | 1.00 | 90.79 |
| 5015 | CB | ALA | D | 540 | 83.843 | 97.768 | 191.030 | 1.00 | 120.12 |
| 5016 | C | ALA | D | 540 | 83.155 | 99.291 | 189.179 | 1.00 | 83.01 |
| 5017 | O | ALA | D | 540 | 83.133 | 99.004 | 187.988 | 1.00 | 116.07 |
| 5018 | N | VAL | D | 541 | 83.596 | 100.459 | 189.629 | 1.00 | 73.02 |
| 5019 | CA | VAL | D | 541 | 84.107 | 101.491 | 188.735 | 1.00 | 89.93 |
| 5020 | CB | VAL | D | 541 | 82.963 | 102.295 | 188.111 | 1.00 | 88.65 |
| 5021 | CG1 | VAL | D | 541 | 82.228 | 103.037 | 189.197 | 1.00 | 84.77 |
| 5022 | CG2 | VAL | D | 541 | 83.498 | 103.274 | 187.069 | 1.00 | 53.86 |
| 5023 | C | VAL | D | 541 | 84.979 | 102.443 | 189.547 | 1.00 | 102.56 |
| 5024 | O | VAL | D | 541 | 85.129 | 102.267 | 190.755 | 1.00 | 153.42 |
| 5025 | N | SER | D | 542 | 85.550 | 103.446 | 188.882 | 1.00 | 109.91 |
| 5026 | CA | SER | D | 542 | 86.397 | 104.444 | 189.533 | 1.00 | 97.55 |
| 5027 | CB | SER | D | 542 | 87.392 | 103.773 | 190.490 | 1.00 | 113.18 |
| 5028 | OG | SER | D | 542 | 88.174 | 102.799 | 189.819 | 1.00 | 118.66 |
| 5029 | C | SER | D | 542 | 87.169 | 105.273 | 188.516 | 1.00 | 76.47 |
| 5030 | O | SER | D | 542 | 87.411 | 104.831 | 187.395 | 1.00 | 131.73 |
| 5031 | N | VAL | D | 543 | 87.548 | 106.481 | 188.909 | 1.00 | 61.96 |
| 5032 | CA | VAL | D | 543 | 88.325 | 107.348 | 188.034 | 1.00 | 111.23 |
| 5033 | CB | VAL | D | 543 | 88.073 | 108.846 | 188.322 | 1.00 | 129.59 |
| 5034 | CG1 | VAL | D | 543 | 88.348 | 109.653 | 187.057 | 1.00 | 63.37 |
| 5035 | CG2 | VAL | D | 543 | 86.642 | 109.072 | 188.868 | 1.00 | 29.47 |
| 5036 | C | VAL | D | 543 | 89.805 | 107.068 | 188.309 | 1.00 | 152.73 |
| 5037 | O | VAL | D | 543 | 90.167 | 106.634 | 189.406 | 1.00 | 171.72 |
| 5038 | N | ASN | D | 544 | 90.655 | 107.326 | 187.320 | 1.00 | 149.81 |
| 5039 | CA | ASN | D | 544 | 92.091 | 107.096 | 187.461 | 1.00 | 158.60 |
| 5040 | CB | ASN | D | 544 | 92.644 | 107.880 | 188.658 | 1.00 | 160.72 |
| 5041 | CG | ASN | D | 544 | 92.354 | 109.369 | 188.568 | 1.00 | 151.01 |
| 5042 | OD1 | ASN | D | 544 | 92.701 | 110.027 | 187.586 | 1.00 | 153.71 |
| 5043 | ND2 | ASN | D | 544 | 91.716 | 109.907 | 189.600 | 1.00 | 105.81 |
| 5044 | C | ASN | D | 544 | 92.415 | 105.605 | 187.627 | 1.00 | 162.71 |
| 5045 | O | ASN | D | 544 | 93.206 | 105.090 | 186.810 | 1.00 | 168.57 |
| 5046 | OXT | ASN | D | 544 | 91.886 | 104.965 | 188.563 | 1.00 | 91.11 |
| 5047 | C1 | NAG | D | 694 | 45.181 | 116.572 | 187.768 | 1.00 | 63.34 |
| 5048 | C2 | NAG | D | 694 | 45.182 | 115.814 | 186.435 | 1.00 | 51.52 |
| 5049 | N2 | NAG | D | 694 | 43.887 | 115.931 | 185.794 | 1.00 | 71.95 |
| 5050 | C7 | NAG | D | 694 | 43.803 | 116.134 | 184.485 | 1.00 | 76.86 |
| 5051 | O7 | NAG | D | 694 | 43.995 | 115.243 | 183.656 | 1.00 | 109.89 |
| 5052 | C8 | NAG | D | 694 | 43.455 | 117.540 | 184.026 | 1.00 | 79.33 |
| 5053 | C3 | NAG | D | 694 | 45.516 | 114.334 | 186.657 | 1.00 | 53.42 |
| 5054 | O3 | NAG | D | 694 | 45.596 | 113.666 | 185.403 | 1.00 | 84.81 |
| 5055 | C4 | NAG | D | 694 | 46.845 | 114.203 | 187.408 | 1.00 | 72.20 |
| 5056 | O4 | NAG | D | 694 | 47.134 | 112.810 | 187.695 | 1.00 | 113.39 |
| 5057 | C5 | NAG | D | 694 | 46.776 | 115.015 | 188.712 | 1.00 | 81.05 |
| 5058 | O5 | NAG | D | 694 | 46.445 | 116.403 | 188.432 | 1.00 | 76.78 |
| 5059 | C6 | NAG | D | 694 | 48.102 | 115.016 | 189.457 | 1.00 | 135.14 |
| 5060 | O6 | NAG | D | 694 | 49.101 | 115.734 | 188.742 | 1.00 | 168.86 |
| 5061 | C1 | NAG | D | 695 | 48.197 | 112.221 | 187.004 | 1.00 | 162.56 |
| 5062 | C2 | NAG | D | 695 | 49.047 | 111.359 | 187.959 | 1.00 | 161.99 |
| 5063 | N2 | NAG | D | 695 | 49.643 | 112.180 | 188.999 | 1.00 | 176.87 |
| 5064 | C7 | NAG | D | 695 | 49.835 | 111.675 | 190.216 | 1.00 | 158.22 |
| 5065 | O7 | NAG | D | 695 | 50.822 | 111.000 | 190.511 | 1.00 | 158.06 |
| 5066 | C8 | NAG | D | 695 | 48.769 | 111.953 | 191.265 | 1.00 | 108.38 |
| 5067 | C3 | NAG | D | 695 | 50.146 | 110.631 | 187.168 | 1.00 | 149.27 |
| 5068 | O3 | NAG | D | 695 | 50.894 | 109.785 | 188.028 | 1.00 | 157.06 |
| 5069 | C4 | NAG | D | 695 | 49.521 | 109.804 | 186.043 | 1.00 | 139.32 |
| 5070 | O4 | NAG | D | 695 | 50.565 | 109.173 | 185.262 | 1.00 | 134.98 |
| 5071 | C5 | NAG | D | 695 | 48.678 | 110.741 | 185.160 | 1.00 | 171.70 |
| 5072 | O5 | NAG | D | 695 | 47.654 | 111.397 | 185.953 | 1.00 | 147.36 |
| 5073 | C6 | NAG | D | 695 | 47.969 | 110.032 | 184.022 | 1.00 | 163.04 |
| 5074 | O6 | NAG | D | 695 | 47.105 | 110.920 | 183.327 | 1.00 | 154.34 |
| 5075 | C1 | MAN | D | 696 | 50.684 | 107.790 | 185.360 | 1.00 | 151.20 |
| 5076 | C2 | MAN | D | 696 | 51.539 | 107.258 | 184.202 | 1.00 | 196.02 |
| 5077 | O2 | MAN | D | 696 | 52.807 | 107.901 | 184.205 | 1.00 | 199.76 |
| 5078 | C3 | MAN | D | 696 | 51.725 | 105.743 | 184.350 | 1.00 | 186.47 |
| 5079 | O3 | MAN | D | 696 | 52.588 | 105.259 | 183.329 | 1.00 | 168.62 |
| 5080 | C4 | MAN | D | 696 | 52.308 | 105.414 | 185.732 | 1.00 | 184.79 |
| 5081 | O4 | MAN | D | 696 | 52.356 | 104.004 | 185.908 | 1.00 | 169.18 |
| 5082 | C5 | MAN | D | 696 | 51.437 | 106.043 | 186.831 | 1.00 | 155.53 |
| 5083 | O5 | MAN | D | 696 | 51.309 | 107.471 | 186.616 | 1.00 | 152.57 |
| 5084 | C6 | MAN | D | 696 | 51.972 | 105.838 | 188.246 | 1.00 | 138.84 |
| 5085 | O6 | MAN | D | 696 | 53.387 | 105.694 | 188.271 | 1.00 | 130.94 |
| 5086 | C1 | CPS | E | 101 | 26.312 | 116.112 | 182.219 | 1.00 | 5.42 |
| 5087 | C2 | CPS | E | 101 | 25.430 | 116.494 | 183.392 | 1.00 | 42.91 |
| 5088 | C3 | CPS | E | 101 | 25.569 | 114.197 | 184.531 | 1.00 | 20.32 |
| 5089 | C4 | CPS | E | 101 | 25.066 | 112.803 | 185.246 | 1.00 | 43.57 |
| 5090 | C5 | CPS | E | 101 | 24.092 | 113.131 | 186.307 | 1.00 | 40.19 |
| 5091 | C6 | CPS | E | 101 | 23.154 | 114.241 | 185.856 | 1.00 | 48.37 |
| 5092 | C7 | CPS | E | 101 | 22.219 | 114.521 | 186.964 | 1.00 | 35.77 |
| 5093 | C8 | CPS | E | 101 | 22.186 | 113.112 | 187.776 | 1.00 | 46.50 |
| 5094 | C9 | CPS | E | 101 | 23.212 | 112.183 | 186.927 | 1.00 | 21.17 |
| 5095 | C10 | CPS | E | 101 | 25.033 | 113.895 | 187.520 | 1.00 | 6.18 |
| 5096 | C11 | CPS | E | 101 | 26.201 | 117.156 | 184.612 | 1.00 | 11.25 |
| 5097 | C12 | CPS | E | 101 | 25.595 | 115.700 | 180.948 | 1.00 | 105.46 |
| 5098 | C13 | CPS | E | 101 | 24.630 | 116.690 | 180.447 | 1.00 | 51.23 |
| 5099 | C14 | CPS | E | 101 | 23.589 | 117.028 | 181.573 | 1.00 | 47.83 |
| 5100 | C15 | CPS | E | 101 | 24.383 | 117.491 | 182.865 | 1.00 | 15.76 |
| 5101 | C16 | CPS | E | 101 | 23.421 | 117.851 | 183.910 | 1.00 | 38.47 |
| 5102 | C17 | CPS | E | 101 | 22.681 | 116.741 | 184.654 | 1.00 | 63.19 |
| 5103 | C18 | CPS | E | 101 | 23.637 | 115.556 | 185.273 | 1.00 | 9.51 |
| 5104 | C19 | CPS | E | 101 | 24.660 | 115.277 | 183.985 | 1.00 | 10.09 |
| 5105 | C20 | CPS | E | 101 | 23.634 | 111.029 | 187.910 | 1.00 | 16.56 |
| 5106 | C21 | CPS | E | 101 | 24.712 | 110.080 | 187.465 | 1.00 | 66.60 |
| 5107 | C22 | CPS | E | 101 | 22.307 | 110.241 | 188.314 | 1.00 | 66.32 |
| 5108 | C23 | CPS | E | 101 | 22.401 | 109.119 | 189.237 | 1.00 | 40.09 |
| 5109 | O2 | CPS | E | 101 | 23.891 | 116.247 | 179.167 | 1.00 | 70.91 |
| 5110 | O3 | CPS | E | 101 | 21.848 | 116.073 | 183.892 | 1.00 | 56.49 |
| 5111 | O4 | CPS | E | 101 | 24.411 | 112.252 | 184.392 | 1.00 | 89.45 |
| 5112 | C1 | CHA | E | 102 | 30.416 | 120.373 | 183.529 | 1.00 | 52.35 |
| 5113 | C2 | CHA | E | 102 | 29.113 | 120.721 | 182.838 | 1.00 | 60.47 |
| 5114 | C3 | CHA | E | 102 | 29.802 | 119.956 | 180.443 | 1.00 | 57.28 |
| 5115 | C4 | CHA | E | 102 | 30.034 | 120.057 | 178.819 | 1.00 | 61.04 |
| 5116 | C5 | CHA | E | 102 | 28.820 | 120.636 | 178.155 | 1.00 | 63.59 |
| 5117 | C6 | CHA | E | 102 | 28.187 | 121.729 | 178.965 | 1.00 | 68.16 |
| 5118 | C7 | CHA | E | 102 | 26.964 | 122.165 | 178.185 | 1.00 | 71.21 |
| 5119 | C8 | CHA | E | 102 | 27.365 | 121.780 | 176.626 | 1.00 | 72.34 |
| 5120 | C9 | CHA | E | 102 | 28.850 | 121.169 | 176.840 | 1.00 | 68.39 |
| 5121 | C10 | CHA | E | 102 | 27.634 | 119.363 | 178.205 | 1.00 | 37.85 |
| 5122 | C11 | CHA | E | 102 | 28.076 | 119.543 | 182.924 | 1.00 | 58.04 |
| 5123 | C12 | CHA | E | 102 | 31.426 | 121.484 | 183.528 | 1.00 | 61.79 |
| 5124 | C13 | CHA | E | 102 | 30.933 | 122.729 | 184.176 | 1.00 | 64.51 |
| 5125 | C14 | CHA | E | 102 | 29.611 | 123.203 | 183.499 | 1.00 | 70.75 |
| 5126 | C15 | CHA | E | 102 | 28.577 | 121.993 | 183.504 | 1.00 | 67.54 |
| 5127 | C16 | CHA | E | 102 | 27.325 | 122.461 | 182.894 | 1.00 | 75.83 |
| 5128 | C17 | CHA | E | 102 | 27.241 | 122.710 | 181.390 | 1.00 | 74.20 |
| 5129 | C18 | CHA | E | 102 | 27.880 | 121.531 | 180.440 | 1.00 | 63.18 |
| 5130 | C19 | CHA | E | 102 | 29.262 | 121.064 | 181.304 | 1.00 | 59.69 |
| 5131 | C20 | CHA | E | 102 | 29.225 | 120.329 | 175.558 | 1.00 | 70.63 |
| 5132 | C21 | CHA | E | 102 | 30.563 | 119.632 | 175.630 | 1.00 | 85.68 |
| 5133 | C22 | CHA | E | 102 | 29.152 | 121.269 | 174.231 | 1.00 | 65.66 |
| 5134 | C23 | CHA | E | 102 | 29.532 | 120.583 | 172.993 | 1.00 | 60.28 |
| 5135 | O2 | CHA | E | 102 | 31.918 | 123.917 | 184.175 | 1.00 | 72.67 |
| 5136 | O3 | CHA | E | 102 | 27.885 | 123.807 | 181.001 | 1.00 | 76.62 |
| 5137 | O4 | CHA | E | 102 | 30.868 | 120.933 | 178.735 | 1.00 | 61.67 |
| 5138 | C24 | CHA | E | 102 | 30.917 | 120.626 | 172.750 | 1.00 | 70.84 |
| 5139 | O5 | CHA | E | 102 | 31.747 | 121.244 | 173.427 | 1.00 | 86.94 |
| 5140 | N25 | CHA | E | 102 | 31.345 | 119.924 | 171.722 | 1.00 | 71.63 |
| 5141 | C25 | CHA | E | 102 | 35.585 | 118.656 | 169.928 | 1.00 | 97.77 |
| 5142 | C26 | CHA | E | 102 | 37.184 | 119.547 | 171.582 | 1.00 | 101.45 |
| 5143 | C27 | CHA | E | 102 | 32.967 | 119.825 | 171.396 | 1.00 | 81.57 |
| 5144 | C28 | CHA | E | 102 | 33.756 | 119.017 | 172.519 | 1.00 | 73.52 |
| 5145 | C29 | CHA | E | 102 | 35.099 | 118.469 | 172.437 | 1.00 | 83.76 |
| 5146 | N1 | CHA | E | 102 | 36.183 | 118.470 | 171.311 | 1.00 | 101.30 |
| 5147 | C30 | CHA | E | 102 | 36.851 | 117.076 | 171.401 | 1.00 | 103.79 |
| 5148 | C31 | CHA | E | 102 | 37.861 | 116.562 | 172.339 | 1.00 | 103.99 |
| 5149 | C32 | CHA | E | 102 | 38.216 | 115.072 | 172.105 | 1.00 | 101.66 |
| 5150 | S1 | CHA | E | 102 | 37.044 | 114.052 | 172.308 | 1.00 | 98.10 |
| 5151 | O6 | CHA | E | 102 | 37.726 | 112.791 | 172.029 | 1.00 | 94.90 |
| 5152 | O7 | CHA | E | 102 | 36.530 | 114.113 | 173.536 | 1.00 | 94.04 |
| 5153 | O8 | CHA | E | 102 | 36.102 | 114.281 | 171.234 | 1.00 | 98.37 |
| 5154 | C1 | CPS | E | 103 | 32.216 | 113.269 | 184.109 | 1.00 | 78.09 |
| 5155 | C2 | CPS | E | 103 | 30.907 | 113.504 | 184.849 | 1.00 | 57.43 |
| 5156 | C3 | CPS | E | 103 | 31.645 | 115.680 | 185.963 | 1.00 | 11.62 |
| 5157 | C4 | CPS | E | 103 | 31.940 | 116.766 | 187.130 | 1.00 | 85.58 |
| 5158 | C5 | CPS | E | 103 | 30.773 | 116.851 | 188.064 | 1.00 | 75.94 |
| 5159 | C6 | CPS | E | 103 | 30.148 | 115.491 | 188.287 | 1.00 | 54.40 |
| 5160 | C7 | CPS | E | 103 | 29.007 | 115.648 | 189.227 | 1.00 | 38.04 |
| 5161 | C8 | CPS | E | 103 | 29.435 | 116.990 | 190.053 | 1.00 | 99.76 |
| 5162 | C9 | CPS | E | 103 | 30.861 | 117.406 | 189.365 | 1.00 | 99.01 |
| 5163 | C10 | CPS | E | 103 | 29.523 | 117.654 | 187.165 | 1.00 | 26.64 |
| 5164 | C11 | CPS | E | 103 | 29.830 | 114.271 | 183.974 | 1.00 | 86.48 |
| 5165 | C12 | CPS | E | 103 | 33.215 | 112.383 | 184.837 | 1.00 | 41.22 |
| 5166 | C13 | CPS | E | 103 | 32.685 | 111.063 | 185.227 | 1.00 | 35.35 |
| 5167 | C14 | CPS | E | 103 | 31.396 | 111.225 | 186.094 | 1.00 | 60.04 |
| 5168 | C15 | CPS | E | 103 | 30.372 | 112.125 | 185.283 | 1.00 | 60.63 |
| 5169 | C16 | CPS | E | 103 | 29.149 | 112.239 | 186.070 | 1.00 | 26.81 |
| 5170 | C17 | CPS | E | 103 | 29.099 | 113.110 | 187.300 | 1.00 | 68.24 |
| 5171 | C18 | CPS | E | 103 | 29.746 | 114.600 | 187.134 | 1.00 | 25.03 |
| 5172 | C19 | CPS | E | 103 | 31.088 | 114.294 | 186.172 | 1.00 | 47.93 |
| 5173 | C20 | CPS | E | 103 | 31.120 | 118.945 | 189.692 | 1.00 | 143.80 |
| 5174 | C21 | CPS | E | 103 | 32.295 | 119.650 | 189.055 | 1.00 | 182.24 |
| 5175 | C22 | CPS | E | 103 | 31.182 | 119.088 | 191.263 | 1.00 | 162.60 |
| 5176 | C23 | CPS | E | 103 | 31.415 | 120.407 | 191.794 | 1.00 | 169.03 |
| 5177 | O2 | CPS | E | 103 | 33.678 | 110.186 | 185.988 | 1.00 | 96.90 |
| 5178 | O3 | CPS | E | 103 | 29.754 | 112.603 | 188.293 | 1.00 | 39.74 |
| 5179 | O4 | CPS | E | 103 | 32.821 | 116.237 | 187.761 | 1.00 | 105.48 |
| 5180 | C1 | CPS | E | 104 | 20.969 | 119.198 | 190.086 | 1.00 | 129.45 |
| 5181 | C2 | CPS | E | 104 | 21.575 | 119.457 | 188.703 | 1.00 | 48.78 |
| 5182 | C3 | CPS | E | 104 | 23.879 | 120.110 | 189.583 | 1.00 | 31.80 |
| 5183 | C4 | CPS | E | 104 | 25.238 | 120.987 | 189.816 | 1.00 | 107.94 |
| 5184 | C5 | CPS | E | 104 | 25.780 | 121.443 | 188.506 | 1.00 | 77.04 |
| 5185 | C6 | CPS | E | 104 | 24.660 | 121.867 | 187.572 | 1.00 | 43.50 |
| 5186 | C7 | CPS | E | 104 | 25.269 | 122.326 | 186.286 | 1.00 | 45.84 |
| 5187 | C8 | CPS | E | 104 | 26.760 | 122.793 | 186.764 | 1.00 | 97.93 |
| 5188 | C9 | CPS | E | 104 | 26.767 | 122.465 | 188.375 | 1.00 | 99.02 |
| 5189 | C10 | CPS | E | 104 | 26.370 | 119.995 | 187.748 | 1.00 | 36.89 |
| 5190 | C11 | CPS | E | 104 | 22.163 | 118.151 | 188.039 | 1.00 | 55.12 |
| 5191 | C12 | CPS | E | 104 | 20.228 | 120.384 | 190.698 | 1.00 | 194.25 |
| 5192 | C13 | CPS | E | 104 | 19.160 | 120.964 | 189.834 | 1.00 | 176.18 |
| 5193 | C14 | CPS | E | 104 | 19.732 | 121.345 | 188.421 | 1.00 | 77.57 |
| 5194 | C15 | CPS | E | 104 | 20.460 | 120.068 | 187.809 | 1.00 | 66.30 |
| 5195 | C16 | CPS | E | 104 | 20.958 | 120.428 | 186.479 | 1.00 | 100.76 |
| 5196 | C17 | CPS | E | 104 | 22.157 | 121.368 | 186.362 | 1.00 | 79.09 |
| 5197 | C18 | CPS | E | 104 | 23.461 | 120.969 | 187.291 | 1.00 | 12.31 |
| 5198 | C19 | CPS | E | 104 | 22.712 | 120.523 | 188.734 | 1.00 | 52.66 |
| 5199 | C20 | CPS | E | 104 | 28.295 | 122.305 | 188.812 | 1.00 | 102.44 |
| 5200 | C21 | CPS | E | 104 | 28.602 | 121.819 | 190.202 | 1.00 | 40.31 |
| 5201 | C22 | CPS | E | 104 | 29.033 | 123.678 | 188.532 | 1.00 | 97.92 |
| 5202 | C23 | CPS | E | 104 | 30.441 | 123.753 | 188.854 | 1.00 | 59.31 |
| 5203 | O2 | CPS | E | 104 | 18.441 | 122.194 | 190.434 | 1.00 | 131.97 |
| 5204 | O3 | CPS | E | 104 | 21.885 | 122.594 | 186.735 | 1.00 | 102.52 |
| 5205 | O4 | CPS | E | 104 | 24.841 | 121.994 | 190.360 | 1.00 | 113.49 |
| 5206 | C1 | CPS | E | 105 | 23.987 | 110.282 | 194.190 | 1.00 | 124.07 |
| 5207 | C2 | CPS | E | 105 | 23.504 | 111.201 | 193.051 | 1.00 | 179.02 |
| 5208 | C3 | CPS | E | 105 | 25.048 | 113.150 | 193.677 | 1.00 | 146.55 |
| 5209 | C4 | CPS | E | 105 | 25.528 | 114.656 | 194.049 | 1.00 | 150.36 |
| 5210 | C5 | CPS | E | 105 | 24.986 | 115.634 | 193.073 | 1.00 | 150.31 |
| 5211 | C6 | CPS | E | 105 | 23.542 | 115.307 | 192.719 | 1.00 | 151.98 |
| 5212 | C7 | CPS | E | 105 | 23.086 | 116.345 | 191.744 | 1.00 | 129.40 |
| 5213 | C8 | CPS | E | 105 | 24.069 | 117.613 | 192.103 | 1.00 | 138.46 |
| 5214 | C9 | CPS | E | 105 | 25.016 | 117.025 | 193.307 | 1.00 | 158.19 |
| 5215 | C10 | CPS | E | 105 | 25.809 | 115.351 | 191.555 | 1.00 | 57.99 |
| 5216 | C11 | CPS | E | 105 | 24.253 | 110.935 | 191.681 | 1.00 | 168.46 |
| 5217 | C12 | CPS | E | 105 | 23.199 | 110.388 | 195.482 | 1.00 | 171.52 |
| 5218 | C13 | CPS | E | 105 | 21.738 | 110.182 | 195.325 | 1.00 | 189.12 |
| 5219 | C14 | CPS | E | 105 | 21.142 | 111.149 | 194.226 | 1.00 | 180.63 |
| 5220 | C15 | CPS | E | 105 | 21.981 | 110.976 | 192.870 | 1.00 | 180.59 |
| 5221 | C16 | CPS | E | 105 | 21.395 | 111.864 | 191.831 | 1.00 | 152.08 |
| 5222 | C17 | CPS | E | 105 | 21.590 | 113.385 | 191.907 | 1.00 | 149.04 |
| 5223 | C18 | CPS | E | 105 | 23.124 | 113.895 | 192.254 | 1.00 | 131.84 |
| 5224 | C19 | CPS | E | 105 | 23.632 | 112.728 | 193.398 | 1.00 | 173.79 |
| 5225 | C20 | CPS | E | 105 | 26.373 | 117.881 | 193.364 | 1.00 | 141.17 |
| 5226 | C21 | CPS | E | 105 | 27.491 | 117.441 | 194.289 | 1.00 | 61.94 |
| 5227 | C22 | CPS | E | 105 | 26.007 | 119.377 | 193.665 | 1.00 | 163.34 |
| 5228 | C23 | CPS | E | 105 | 27.114 | 120.298 | 193.745 | 1.00 | 162.44 |
| 5229 | O2 | CPS | E | 105 | 20.934 | 110.337 | 196.634 | 1.00 | 146.47 |
| 5230 | O3 | CPS | E | 105 | 20.861 | 113.951 | 192.840 | 1.00 | 182.07 |
| 5231 | O4 | CPS | E | 105 | 24.940 | 114.899 | 195.082 | 1.00 | 183.30 |
| 5232 | S | SO4 | F | 101 | 26.461 | 117.594 | 160.481 | 1.00 | 117.04 |
| 5233 | O1 | SO4 | F | 101 | 26.028 | 117.364 | 161.888 | 1.00 | 114.89 |
| 5234 | O2 | SO4 | F | 101 | 25.645 | 118.674 | 159.871 | 1.00 | 116.85 |
| 5235 | O3 | SO4 | F | 101 | 27.889 | 117.990 | 160.442 | 1.00 | 104.02 |
| 5236 | O4 | SO4 | F | 101 | 26.264 | 116.346 | 159.701 | 1.00 | 116.98 |
| 5237 | S | SO4 | F | 102 | 30.691 | 115.815 | 152.464 | 1.00 | 84.09 |
| 5238 | O1 | SO4 | F | 102 | 31.425 | 115.760 | 153.735 | 1.00 | 75.95 |
| 5239 | O2 | SO4 | F | 102 | 30.165 | 117.185 | 152.282 | 1.00 | 88.58 |
| 5240 | O3 | SO4 | F | 102 | 31.591 | 115.536 | 151.339 | 1.00 | 88.44 |
| 5241 | O4 | SO4 | F | 102 | 29.608 | 114.799 | 152.483 | 1.00 | 82.42 |
| 5242 | S | SO4 | F | 103 | 21.641 | 101.569 | 151.307 | 1.00 | 92.25 |
| 5243 | O1 | SO4 | F | 103 | 22.530 | 100.415 | 151.659 | 1.00 | 115.82 |
| 5244 | O2 | SO4 | F | 103 | 21.490 | 102.482 | 152.472 | 1.00 | 115.45 |
| 5245 | O3 | SO4 | F | 103 | 22.255 | 102.282 | 150.149 | 1.00 | 112.57 |
| 5246 | O4 | SO4 | F | 103 | 20.304 | 101.049 | 150.949 | 1.00 | 111.09 |
| 5247 | S | SO4 | F | 104 | 63.588 | 107.320 | 177.755 | 1.00 | 90.55 |
| 5248 | O1 | SO4 | F | 104 | 64.167 | 105.957 | 177.946 | 1.00 | 104.42 |
| 5249 | O2 | SO4 | F | 104 | 63.018 | 107.748 | 179.075 | 1.00 | 101.96 |
| 5250 | O3 | SO4 | F | 104 | 64.623 | 108.285 | 177.316 | 1.00 | 107.44 |
| 5251 | O4 | SO4 | F | 104 | 62.568 | 107.276 | 176.668 | 1.00 | 101.74 |
| 5252 | S | SO4 | F | 105 | 38.290 | 100.112 | 181.573 | 1.00 | 94.13 |
| 5253 | O1 | SO4 | F | 105 | 39.110 | 99.271 | 182.495 | 1.00 | 100.20 |
| 5254 | O2 | SO4 | F | 105 | 36.859 | 99.943 | 181.952 | 1.00 | 108.28 |
| 5255 | O3 | SO4 | F | 105 | 38.642 | 101.562 | 181.681 | 1.00 | 98.51 |
| 5256 | O4 | SO4 | F | 105 | 38.529 | 99.646 | 180.172 | 1.00 | 109.17 |

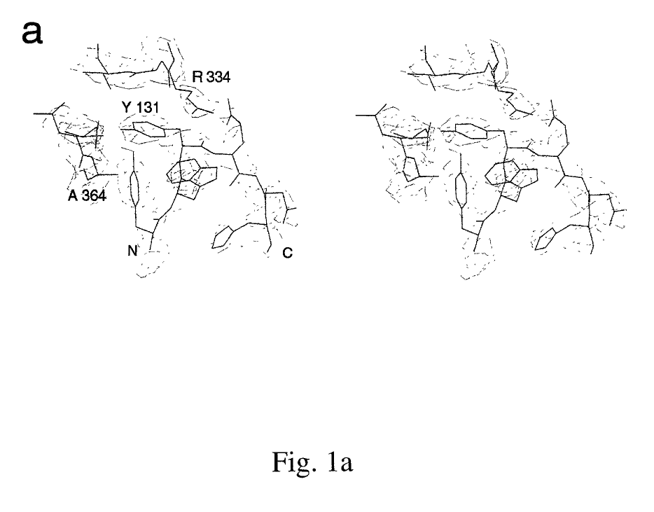

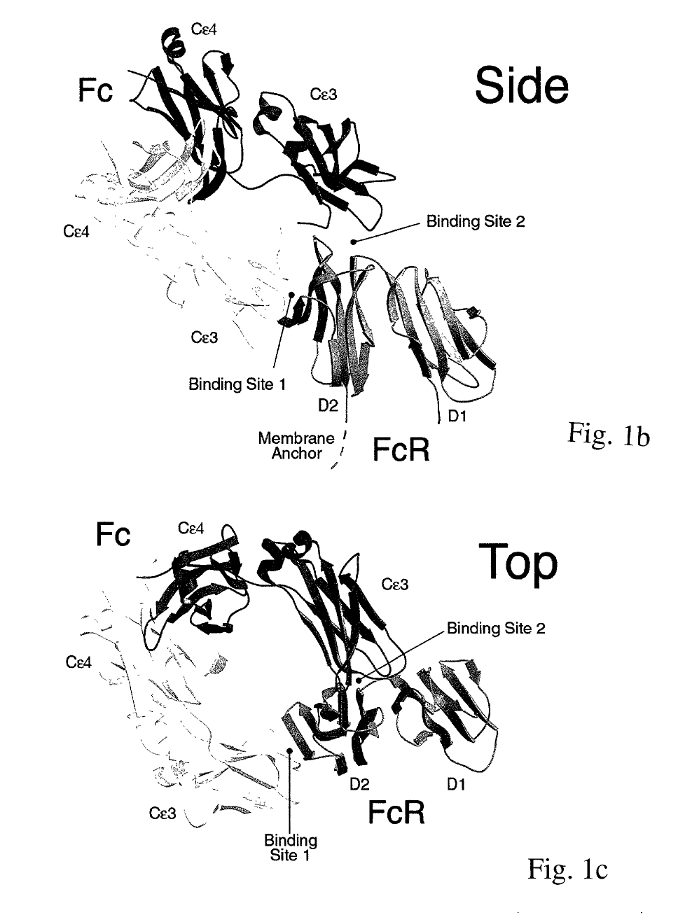

As used herein, an atomic coordinate, also referred to herein as a structure coordinate or coordinate, is a mathematical coordinate derived from mathematical equations related to the patterns obtained on diffraction of X-rays by the atoms of a protein or complex crystal. The diffraction data are typically used to calculate an electron density map, such as that shown in FIG. 1, which is used to establish the positions of the individual atoms within the unit cell of the crystal. A model that substantially represents the atomic coordinates specified in Table 1 includes not only models that literally represent the coordinates but also models representing a coordinate transformation of such atomic coordinates, for example, by changing the spatial orientation of the coordinates.

The present invention also includes a 3-D model that is a modification of a 3-D model that substantially represents the atomic coordinates specified in Table 1. As used herein, a modification, also referred to herein as a model modification, is a model that represents a complex between a protein that binds to a Fc domain of an antibody and an antibody Fc regon that binds to a Fc receptor protein. A model modification includes, but is not limited to: a refinement of the model that substantially represents the atomic coordinates specified in Table 1; a model representing a complex between any Fc-binding fragment of a Fc receptor protein and any FcR-binding fragment of an antibody having the atomic coordinates specified in Table 1; a model based on other FcεRIα:Fc-Cε3/Cε4 crystals, such as a model based on one or more of the crystals disclosed in the Examples; a model produced using homology modeling techniques to, for example, incorporate all or any part of the amino acid sequence of another FcR or antibody into a 3-D model substantially representing the atomic coordinates specified in Table 1 or incorporate all or any part of the amino acid sequence of a FcεRIα protein or Fc-Cε3/Cε4 into a 3-D model of a complex between another FcR and antibody; and a modification representing a complex between an FcR and antibody, at least one of which has an altered function, which preferably can be used to design a mutein with an improved function compared to an unmodified protein. As used herein, the term unmodified protein refers to a protein that has not been intentionally subjected to either random or site-directed (i.e., targeted) mutagenesis.

A model of the present invention can be represented in a variety of forms including, but not limited to, listing the coordinates of all atoms comprising the model, providing a physical 3-D model, imaging the model on a computer screen, providing a picture of said model, and deriving a set of coordinates based of a picture of the model, for example by extracting coordinates from a picture or placing a similar immunoglobulin domain into the 3-D model of a human FcεRIα₁₋₁₇₆ protein having SEQ ID NO:2 and deriving a model of the similar domain. Physical 3-D models are tangible and include, but are not limited to, stick models and space-filling models. The phrase “imaging the model on a computer screen” refers to the ability to express (or represent) and manipulate the model on a computer screen using appropriate computer hardware and software technology known to those skilled in the art. Such technology is available from a variety of sources including, for example, Evans and Sutherland, Salt Lake City, Utah, Biosym Technologies, San Diego, Calif., Tripos, Inc., and Molecular Simulations Inc. The phrase “providing a picture of the model” refers to the ability to generate a “hard copy” of the model. Hard copies include both motion and still pictures. Computer screen images and pictures of the model can be visualized in a number of formats including, but not limited to, electron density maps, ribbon diagrams, space-filling representations, a carbon traces, topology diagrams, lists of interatomic vectors, phi/psi/chi angle representations of the coordinates, and contact maps, examples of some of which are in the Figs. Representations of the model can include the entire model or portions thereof. A model can also be represented in a database.

A model of the present invention also defines the space surrounding that model. Such a space can be represented as a mold, or alpha-space, that can be used to predict the shape of a compound that inhibits the binding of a FcR and antibody.

In one embodiment, a model of the present invention identifies the solvent accessibility of amino acid residues of the corresponding proteins in the complex. The solvent accessibilities of the amino acids in the complex between PhFcεRIα₁₋₁₇₆ₘᵤₜ and PhFc-Cε3/Cε4₁₋₂₂₂ are indicated in Table 2.

TABLE 2
com14i_deposit.pdb Residue Exposure
Surface plot for:
structure file = com14h_gen.mtf
coordinate set = com14i.pdb

| | | | TOTAL ACCESSIBLE AREA | | |
|---|---|---|---|---|---|
| segid | resid | resname | residue | mainchain | sidechain |
| A | 1 | VAL | 187.3253 | 57.6982 | 129.6271 |
| A | 2 | PRO | 92.7850 | 27.8208 | 64.9642 |
| A | 3 | GLN | 136.0547 | 22.4120 | 113.6427 |
| A | 4 | LYS | 115.4501 | 16.7110 | 98.7391 |
| A | 5 | PRO | 15.6134 | 5.2823 | 10.3310 |
| A | 6 | LYS | 129.5753 | 2.2724 | 127.3029 |
| A | 7 | VAL | 13.2508 | 10.5326 | 2.7182 |
| A | 8 | SER | 61.2891 | 6.5958 | 54.6932 |
| A | 9 | LEU | 29.3720 | 15.0058 | 14.3663 |
| A | 10 | ASN | 96.3611 | 5.4707 | 90.8904 |
| A | 11 | PRO | 61.5816 | 1.0093 | 60.5723 |
| A | 12 | PRO | 44.6585 | 3.6780 | 40.9805 |
| A | 13 | TRP | 32.1306 | 0.0000 | 32.1306 |
| A | 14 | ASN | 13.9201 | 0.0000 | 13.9201 |
| A | 15 | ARG | 18.9379 | 0.0000 | 18.9379 |
| A | 16 | ILE | 4.0671 | 0.0000 | 4.0671 |
| A | 17 | PHE | 2.5761 | 0.0015 | 2.5746 |
| A | 18 | LYS | 75.4097 | 9.6110 | 65.7987 |
| A | 19 | GLY | 30.4736 | 30.4736 | 0.0000 |
| A | 20 | GLU | 38.0623 | 1.4738 | 36.5885 |
| A | 21 | ASN | 44.5154 | 12.5957 | 31.9196 |
| A | 22 | VAL | 6.0341 | 5.5689 | 0.4652 |
| A | 23 | THR | 30.3454 | 0.0015 | 30.3439 |
| A | 24 | LEU | 1.9937 | 0.0005 | 1.9933 |
| A | 25 | TER | 45.1783 | 0.8036 | 44.3747 |
| A | 26 | CYS | 1.8288 | 1.8288 | 0.0000 |
| A | 27 | ASN | 45.5609 | 16.4355 | 29.1253 |
| A | 28 | GLY | 57.0567 | 57.0567 | 0.0000 |
| A | 29 | ASN | 92.9262 | 33.7771 | 59.1490 |
| A | 30 | ASN | 13.5663 | 9.6698 | 3.8965 |
| A | 31 | PHE | 164.0905 | 20.6501 | 143.4404 |
| A | 32 | PHE | 182.6224 | 29.9619 | 152.6604 |
| A | 33 | GLU | 98.9835 | 23.5598 | 75.4237 |
| A | 34 | VAL | 112.1392 | 35.9284 | 76.2108 |
| A | 35 | SER | 13.8929 | 11.8212 | 2.0717 |
| A | 36 | SER | 61.4988 | 16.1241 | 45.3747 |
| A | 37 | THR | 3.8229 | 1.4419 | 2.3810 |
| A | 38 | LYS | 54.6368 | 1.5373 | 53.0995 |
| A | 39 | TRP | 0.7682 | 0.0026 | 0.7656 |
| A | 40 | PHE | 35.2234 | 0.8384 | 34.3850 |
| A | 41 | HIS | 42.4410 | 4.2641 | 38.1769 |
| A | 42 | ASN | 55.8729 | 34.7289 | 21.1439 |
| A | 43 | GLY | 50.2523 | 50.2523 | 0.0000 |
| A | 44 | SER | 90.0908 | 14.2647 | 75.8261 |
| A | 45 | LEU | 112.2293 | 26.8607 | 85.3687 |
| A | 46 | SER | 33.6534 | 12.6061 | 21.0473 |
| A | 47 | GLU | 173.0167 | 28.6974 | 144.3194 |
| A | 48 | GLU | 52.6512 | 0.9816 | 51.6696 |
| A | 49 | THR | 78.9495 | 4.5450 | 74.4045 |
| A | 50 | ASN | 83.8564 | 1.8107 | 82.0457 |
| A | 51 | SER | 20.5641 | 0.7215 | 19.8427 |
| A | 52 | SER | 44.0129 | 2.6102 | 41.4027 |
| A | 53 | LEU | 23.9390 | 0.2187 | 23.7203 |
| A | 54 | ASN | 93.7074 | 14.6559 | 79.0515 |
| A | 55 | ILE | 14.9901 | 7.9277 | 7.0624 |
| A | 56 | VAL | 77.8026 | 18.1671 | 59.6354 |
| A | 57 | ASN | 72.5436 | 10.8218 | 61.7218 |
| A | 58 | ALA | 0.1748 | 0.1748 | 0.0000 |
| A | 59 | LYS | 78.3995 | 0.3905 | 78.0090 |
| A | 60 | PHE | 13.8474 | 0.0000 | 13.8474 |
| A | 61 | GLU | 71.1840 | 0.7867 | 70.3974 |
| A | 62 | ASP | 37.6798 | 0.0000 | 37.6798 |
| A | 63 | SER | 0.7611 | 0.0000 | 0.7611 |
| A | 64 | GLY | 10.5710 | 10.5710 | 0.0000 |
| A | 65 | GLU | 48.7849 | 0.8856 | 47.8993 |
| A | 66 | TYR | 9.3817 | 0.0000 | 9.3817 |
| A | 67 | LYS | 39.4871 | 0.0208 | 39.4662 |
| A | 68 | CYS | 0.0000 | 0.0000 | 0.0000 |
| A | 69 | GLN | 32.8025 | 0.0000 | 32.8025 |
| A | 70 | HIS | 28.9440 | 3.7554 | 25.1886 |
| A | 71 | GLN | 127.6128 | 34.5779 | 93.0349 |
| A | 72 | GLN | 114.7755 | 18.7035 | 96.0721 |
| A | 73 | VAL | 129.4891 | 13.4243 | 116.0648 |
| A | 74 | ALA | 32.5769 | 10.0706 | 22.5064 |
| A | 75 | GLU | 64.6775 | 8.8568 | 55.8208 |
| A | 76 | SER | 1.9255 | 1.8897 | 0.0358 |
| A | 77 | GLU | 112.0982 | 4.5586 | 107.5397 |
| A | 78 | PRO | 50.1437 | 14.6260 | 35.5177 |
| A | 79 | VAL | 26.4528 | 3.6105 | 22.8422 |
| A | 80 | TYR | 121.0925 | 5.3004 | 115.7921 |
| A | 81 | LEU | 1.8512 | 0.7930 | 1.0581 |
| A | 82 | GLU | 59.7116 | 0.0003 | 59.7113 |
| A | 83 | VAL | 9.5413 | 9.5413 | 0.0000 |
| A | 84 | PHE | 35.6448 | 3.2623 | 32.3825 |
| A | 85 | SER | 24.8318 | 9.3417 | 15.4901 |
| A | 86 | ASP | 22.6050 | 0.0005 | 22.6045 |
| A | 87 | TRP | 25.2208 | 0.6392 | 24.5816 |
| A | 88 | LEU | 3.0061 | 3.0061 | 0.0000 |
| A | 89 | LEU | 4.7629 | 1.9707 | 2.7922 |
| A | 90 | LEU | 0.6339 | 0.6339 | 0.0000 |
| A | 91 | GLN | 0.7211 | 0.0000 | 0.7211 |
| A | 92 | ALA | 1.9224 | 0.9484 | 0.9739 |
| A | 93 | SER | 28.3506 | 16.9666 | 11.3840 |
| A | 94 | ALA | 31.9213 | 3.2557 | 28.6657 |
| A | 95 | GLU | 59.5399 | 4.5153 | 55.0246 |
| A | 96 | VAL | 90.3253 | 19.0678 | 71.2575 |
| A | 97 | VAL | 6.3340 | 1.9033 | 4.4307 |
| A | 98 | MET | 117.6508 | 1.1378 | 116.5130 |
| A | 99 | GLU | 87.6346 | 20.1858 | 67.4487 |
| A | 100 | GLY | 37.5111 | 37.5111 | 0.0000 |
| A | 101 | GLN | 86.3207 | 1.7512 | 84.5695 |
| A | 102 | PRO | 60.8738 | 6.3890 | 54.4848 |
| A | 103 | LEU | 0.4221 | 0.0000 | 0.4221 |
| A | 104 | PHE | 80.0346 | 0.0026 | 80.0320 |
| A | 105 | LEU | 0.1253 | 0.1242 | 0.0011 |
| A | 106 | ARG | 68.1925 | 0.0000 | 68.1925 |
| A | 107 | CYS | 3.4779 | 3.4779 | 0.0000 |
| A | 108 | HIS | 11.8995 | 0.9286 | 10.9708 |
| A | 109 | GLY | 3.1287 | 3.1287 | 0.0000 |
| A | 110 | TRP | 32.3303 | 0.5358 | 31.7945 |
| A | 111 | ARG | 102.0115 | 29.5393 | 72.4722 |
| A | 112 | ASN | 103.7825 | 16.7021 | 87.0804 |
| A | 113 | TRP | 8.0187 | 5.9544 | 2.0643 |
| A | 114 | ASP | 56.0982 | 5.8709 | 50.2273 |
| A | 115 | VAL | 3.8019 | 3.8019 | 0.0000 |
| A | 116 | TYR | 28.0985 | 0.0025 | 28.0959 |
| A | 117 | LYS | 13.8640 | 4.0420 | 9.8220 |
| A | 118 | VAL | 0.0000 | 0.0000 | 0.0000 |
| A | 119 | ILE | 2.9639 | 0.0000 | 2.9639 |
| A | 120 | TYR | 0.0664 | 0.0000 | 0.0664 |
| A | 121 | TYR | 33.2837 | 0.0000 | 33.2837 |
| A | 122 | LYS | 25.5895 | 0.0240 | 25.5655 |
| A | 123 | ASP | 78.1271 | 25.4180 | 52.7091 |
| A | 124 | GLY | 62.3032 | 62.3032 | 0.0000 |
| A | 125 | GLU | 120.1814 | 5.1946 | 114.9868 |
| A | 126 | ALA | 31.3601 | 27.4382 | 3.9219 |
| A | 127 | LEU | 76.9250 | 25.4102 | 51.5147 |
| A | 128 | LYS | 112.4216 | 4.4777 | 107.9440 |
| A | 129 | TYR | 5.7182 | 5.6069 | 0.1112 |
| A | 130 | TRP | 71.2318 | 0.0019 | 71.2299 |
| A | 131 | TYR | 2.3182 | 1.8150 | 0.5032 |
| A | 132 | GLU | 48.8765 | 0.0000 | 48.8765 |
| A | 133 | ASN | 56.4646 | 18.4026 | 38.0620 |
| A | 134 | HIS | 34.7605 | 14.3500 | 20.4105 |
| A | 135 | ALA | 61.0033 | 21.2841 | 39.7192 |
| A | 136 | ILE | 12.9140 | 2.0093 | 10.9047 |
| A | 137 | SER | 71.4379 | 27.1612 | 44.2767 |
| A | 138 | ILE | 24.6119 | 3.4516 | 21.1603 |
| A | 139 | THR | 103.9450 | 8.8762 | 95.0688 |
| A | 140 | ASN | 102.3330 | 12.7166 | 89.6164 |
| A | 141 | ALA | 10.3600 | 10.0810 | 0.2790 |
| A | 142 | ALA | 34.0280 | 8.3960 | 25.6320 |
| A | 143 | VAL | 104.7568 | 10.1018 | 94.6550 |
| A | 144 | GLU | 126.9246 | 18.8779 | 108.0467 |
| A | 145 | ASP | 16.9194 | 0.0000 | 16.9194 |
| A | 146 | SER | 21.5373 | 4.0635 | 17.4739 |
| A | 147 | GLY | 5.8021 | 5.8021 | 0.0000 |
| A | 148 | THR | 32.5295 | 0.0829 | 32.4466 |
| A | 149 | TYR | 0.0642 | 0.0000 | 0.0642 |
| A | 150 | TYR | 43.8958 | 0.0226 | 43.8733 |
| A | 151 | CYS | 0.0000 | 0.0000 | 0.0000 |
| A | 152 | THR | 29.2303 | 0.0000 | 29.2303 |
| A | 153 | GLY | 3.9813 | 3.9813 | 0.0000 |
| A | 154 | LYS | 48.4892 | 0.3347 | 48.1546 |
| A | 155 | VAL | 2.0858 | 0.5817 | 1.5040 |
| A | 156 | TRP | 18.7900 | 7.0495 | 11.7404 |
| A | 157 | GLN | 31.8404 | 12.3565 | 19.4840 |
| A | 158 | LEU | 36.5178 | 1.3940 | 35.1238 |
| A | 159 | ASP | 102.6316 | 23.8299 | 78.8017 |
| A | 160 | TYR | 48.3992 | 8.9529 | 39.4463 |
| A | 161 | GLU | 105.3209 | 20.1949 | 85.1260 |
| A | 162 | SER | 3.4294 | 3.2706 | 0.1588 |
| A | 163 | GLU | 82.4632 | 5.8768 | 76.5864 |
| A | 164 | PRO | 93.8712 | 16.5130 | 77.3582 |
| A | 165 | LEU | 14.1332 | 2.3506 | 11.7826 |
| A | 166 | ASN | 40.8356 | 7.7631 | 33.0724 |
| A | 167 | ILE | 1.0917 | 1.0901 | 0.0016 |
| A | 168 | THR | 60.5937 | 0.4273 | 60.1664 |
| A | 169 | VAL | 30.1152 | 27.9591 | 2.1560 |
| A | 170 | ILE | 90.8904 | 5.5150 | 85.3753 |
| A | 171 | LYS | 136.7114 | 19.0209 | 117.6905 |
| A | 172 | ALA | 86.7389 | 24.6924 | 62.0465 |
| A | 173 | PRO | 192.4729 | 58.6006 | 133.8723 |
| A | 221 | NAG | 140.3112 | 0.0000 | 140.3112 |
| A | 222 | NAG | 177.5229 | 0.0000 | 177.5229 |
| A | 223 | MAN | 225.6042 | 0.0000 | 225.6042 |
| A | 224 | FUC | 193.3727 | 0.0000 | 193.3727 |
| A | 242 | NAG | 142.0058 | 0.0000 | 142.0058 |
| A | 243 | NAG | 139.1866 | 0.0000 | 139.1866 |
| A | 244 | MAN | 61.6458 | 0.0000 | 61.6458 |
| A | 245 | MAN | 221.3300 | 0.0000 | 221.3300 |
| A | 246 | MAN | 162.9047 | 0.0000 | 162.9047 |
| A | 366 | NAG | 163.0167 | 0.0000 | 163.0167 |
| A | 367 | NAG | 271.0832 | 0.0000 | 271.0832 |
| A | 369 | FUC | 170.1425 | 0.0000 | 170.1425 |
| B | 328 | PRO | 172.3911 | 55.4048 | 116.9864 |
| B | 329 | CYS | 28.4788 | 7.8618 | 20.6170 |
| B | 330 | ASP | 92.8612 | 15.7041 | 77.1570 |
| B | 331 | SER | 27.0273 | 1.1252 | 25.9021 |
| B | 332 | ASN | 34.9448 | 0.0015 | 34.9432 |
| B | 333 | PRO | 0.0000 | 0.0000 | 0.0000 |
| B | 334 | ARG | 56.1569 | 7.6844 | 48.4725 |
| B | 335 | GLY | 1.1847 | 1.1847 | 0.0000 |
| B | 336 | VAL | 0.0012 | 0.0012 | 0.0000 |
| B | 337 | SER | 15.0140 | 0.0510 | 14.9630 |
| B | 338 | ALA | 5.2899 | 4.7848 | 0.5050 |
| B | 339 | TYR | 51.0452 | 3.6676 | 47.3776 |
| B | 340 | LEU | 29.3001 | 25.9417 | 3.3584 |
| B | 341 | SER | 45.2273 | 8.8444 | 36.3829 |
| B | 342 | ARG | 88.6974 | 12.2167 | 76.4807 |
| B | 343 | PRO | 8.9769 | 8.9769 | 0.0000 |
| B | 344 | SER | 38.4813 | 6.2806 | 32.2008 |
| B | 345 | PRO | 14.1279 | 3.1946 | 10.9334 |
| B | 346 | PHE | 47.7276 | 0.0000 | 47.7276 |
| B | 347 | ASP | 47.1591 | 0.0000 | 47.1591 |
| B | 348 | LEU | 7.6413 | 0.3974 | 7.2439 |
| B | 349 | PHE | 17.8265 | 8.5197 | 9.3068 |
| B | 350 | ILE | 46.3116 | 12.5396 | 33.7719 |
| B | 351 | ARG | 138.4411 | 22.8644 | 115.5766 |
| B | 352 | LYS | 138.7623 | 15.9014 | 122.8608 |
| B | 353 | SER | 48.1246 | 7.3438 | 40.7808 |
| B | 354 | PRO | 3.8128 | 1.3726 | 2.4402 |
| B | 355 | THR | 55.0488 | 11.3329 | 43.7158 |
| B | 356 | ILE | 0.4059 | 0.4059 | 0.0000 |
| B | 357 | THR | 39.8398 | :L.4897 | 38.3501 |
| B | 358 | CYS | 0.3982 | 0.0000 | 0.3982 |
| B | 359 | LEU | 21.2325 | 0.0000 | 21.2325 |
| B | 360 | VAL | 0.6920 | 0.0000 | 0.6920 |
| B | 361 | VAL | 0.7258 | 0.0000 | 0.7258 |
| B | 362 | ASP | 6.8200 | 0.2493 | 6.5707 |
| B | 363 | LEU | 4.2335 | 0.0000 | 4.2335 |
| B | 364 | ALA | 3.3493 | 2.6950 | 0.6543 |
| B | 365 | PRO | 82.6759 | 12.6105 | 70.0654 |
| B | 366 | SER | 50.6093 | 40.2181 | 10.3912 |
| B | 367 | LYS | 213.9320 | 43.2881 | 170.6439 |
| B | 368 | GLY | 40.8947 | 40.8947 | 0.0000 |
| B | 369 | THR | 86.8598 | 19.7007 | 67.1591 |
| B | 370 | VAL | 15.5137 | 7.3337 | 8.1800 |
| B | 371 | ASN | 81.8915 | 3.4400 | 78.4515 |
| B | 372 | LEU | 22.8095 | 18.5551 | 4.2544 |
| B | 373 | THR | 63.4928 | 3.2688 | 60.2240 |
| B | 374 | TRP | 23.4425 | 13.9341 | 9.5085 |
| B | 375 | SER | 45.2542 | 3.8130 | 41.4412 |
| B | 376 | ARG | 46.0272 | 18.4454 | 27.5818 |
| B | 377 | ALA | 72.9508 | 41.9432 | 31.0077 |
| B | 378 | SER | 61.4249 | 42.7807 | 18.6442 |
| B | 379 | GLY | 62.6791 | 62.6791 | 0.0000 |
| B | 380 | LYS | 117.6325 | 4.9136 | 112.7189 |
| B | 381 | PRO | 121.4544 | 18.6968 | 102.7576 |
| B | 382 | VAL | 56.2720 | 27.9106 | 28.3614 |
| B | 383 | ASN | 91.2257 | 9.8352 | 81.3905 |
| B | 384 | HIS | 171.4010 | 14.9519 | 156.4491 |
| B | 385 | SER | 60.0998 | 34.1545 | 25.9453 |
| B | 386 | THR | 61.8510 | 7.3810 | 54.4700 |
| B | 387 | ARG | 90.5383 | 34.3831 | 56.1553 |
| B | 388 | LYS | 116.7691 | 6.8649 | 109.9042 |
| B | 389 | GLU | 68.1229 | 29.0561 | 39.0668 |
| B | 390 | GLU | 95.8196 | 5.5952 | 90.2244 |
| B | 391 | LYS | 165.8039 | 23.0467 | 142.7572 |
| B | 392 | GLN | 28.9793 | 6.2030 | 22.7763 |
| B | 393 | ARG | 216.3236 | 21.7321 | 194.5916 |
| B | 394 | ASN | 21.4124 | 2.2263 | 19.1861 |
| B | 395 | GLY | 18.5421 | 18.5421 | 0.0000 |
| B | 396 | THR | 1.4681 | 0.8446 | 0.6234 |
| B | 397 | LEU | 27.8812 | 0.0001 | 27.8811 |
| B | 398 | THR | 2.5805 | 0.0070 | 2.5735 |
| B | 399 | VAL | 0.2989 | 0.0000 | 0.2989 |
| B | 400 | THR | 28.7559 | 0.0010 | 28.7549 |
| B | 401 | SER | 1.6019 | 0.0320 | 1.5699 |
| B | 402 | THR | 38.8714 | 1.4031 | 37.4684 |
| B | 403 | LEU | 1.4485 | 0.0026 | 1.4459 |
| B | 404 | PRO | 54.7046 | 5.1244 | 49.5802 |
| B | 405 | VAL | 9.9534 | 9.2686 | 0.6848 |
| B | 406 | GLY | 19.5973 | 19.5973 | 0.0000 |
| B | 407 | THR | 17.5422 | 0.0269 | 17.5153 |
| B | 408 | ARG | 149.2990 | 4.0416 | 145.2574 |
| B | 409 | ASP | 50.9581 | 6.4926 | 44.4655 |
| B | 410 | TRP | 13.6629 | 0.0000 | 13.6629 |
| B | 411 | ILE | 58.9623 | 7.5292 | 51.4331 |
| B | 412 | GLU | 150.4506 | 36.6377 | 113.8129 |
| B | 413 | GLY | 37.5912 | 37.5912 | 0.0000 |
| B | 414 | GLU | 20.7783 | 6.9542 | 13.8241 |
| B | 415 | THR | 41.3262 | 0.7425 | 40.5837 |
| B | 416 | TYR | 9.7756 | 0.0127 | 9.7629 |
| B | 417 | GLN | 62.7741 | 0.0234 | 62.7507 |
| B | 418 | CYS | 0.5661 | 0.4620 | 0.1041 |
| B | 419 | ARG | 116.8504 | 0.4330 | 116.4174 |
| B | 420 | VAL | 3.0810 | 0.0024 | 3.0786 |
| B | 421 | THR | 53.9214 | 2.2639 | 51.6575 |
| B | 422 | HIS | 11.9613 | 3.7559 | 8.2055 |
| B | 423 | PRO | 118.2970 | 36.7814 | 81.5157 |
| B | 424 | HIS | 41.1729 | 9.3203 | 31.8526 |
| B | 425 | LEU | 20.1433 | 18.3272 | 1.8162 |
| B | 426 | PRO | 95.2197 | 47.9729 | 47.2468 |
| B | 427 | ARG | 75.8053 | 12.6035 | 63.2017 |
| B | 428 | ALA | 29.9192 | 13.4376 | 16.4816 |
| B | 429 | LEU | 0.6655 | 0.2491 | 0.4164 |
| B | 430 | MET | 94.7862 | 13.9214 | 80.8648 |
| B | 431 | ARG | 61.2436 | 10.0257 | 51.2179 |
| B | 432 | SER | 65.6617 | 25.5585 | 40.1032 |
| B | 433 | THR | 13.0233 | 5.6973 | 7.3260 |
| B | 434 | THR | 46.6839 | 7.5817 | 39.1022 |
| B | 435 | LYS | 48.5670 | 13.2907 | 35.2763 |
| B | 436 | THR | 47.1262 | 7.7492 | 39.3770 |
| B | 437 | SER | 93.7617 | 15.6507 | 78.1110 |
| B | 438 | GLY | 47.1648 | 47.1648 | 0.0000 |
| B | 439 | PRO | 92.2539 | 11.4315 | 80.8224 |
| B | 440 | ARG | 86.4119 | 32.6025 | 53.8094 |
| B | 441 | ALA | 41.0049 | 5.6703 | 35.3346 |
| B | 442 | ALA | 46.7251 | 10.6945 | 36.0306 |
| B | 443 | PRO | 4.7646 | 4.7646 | 0.0000 |
| B | 444 | GLU | 32.6629 | 0.1921 | 32.4708 |
| B | 445 | VAL | 1.9628 | 0.1088 | 1.8541 |
| B | 446 | TYR | 12.1809 | 1.1154 | 11.0655 |
| B | 447 | ALA | 19.0771 | 18.8089 | 0.2682 |
| B | 448 | PHE | 32.2261 | 5.3355 | 26.8906 |
| B | 449 | ALA | 26.3527 | 17.5706 | 8.7821 |
| B | 450 | THR | 8.1738 | 2.2896 | 5.8842 |
| B | 451 | PRO | 76.5842 | 4.9904 | 71.5938 |
| B | 452 | GLU | 93.8169 | 13.0331 | 80.7838 |
| B | 453 | TRP | 95.9141 | 1.6990 | 94.2151 |
| B | 454 | PRO | 125.8288 | 36.9609 | 88.8679 |
| B | 455 | GLY | 65.7610 | 65.7610 | 0.0000 |
| B | 456 | SER | 42.7528 | 8.4299 | 34.3229 |
| B | 457 | ARG | 182.0093 | 19.8205 | 162.1888 |
| B | 458 | ASP | 66.4899 | 1.5486 | 64.9413 |
| B | 459 | LYS | 148.5472 | 12.9959 | 135.5513 |
| B | 460 | ARG | 41.5604 | 3.1133 | 38.4471 |
| B | 461 | THR | 13.2538 | 1.6604 | 11.5934 |
| B | 462 | LEU | 6.3258 | 0.0649 | 6.2610 |
| B | 463 | ALA | 11.5779 | 0.4739 | 11.1040 |
| B | 464 | CYS | 1.0391 | 1.0391 | 0.0000 |
| B | 465 | LEU | 2.3588 | 0.0000 | 2.3588 |
| B | 466 | ILE | 0.3683 | 0.0000 | 0.3683 |
| B | 467 | GLN | 4.2086 | 0.0000 | 4.2086 |
| B | 468 | ASN | 35.9496 | 5.2065 | 30.7431 |
| B | 469 | PHE | 0.0000 | 0.0000 | 0.0000 |
| B | 470 | MET | 32.7360 | 0.0000 | 32.7360 |
| B | 471 | PRO | 9.7948 | 6.3053 | 3.4896 |
| B | 472 | GLU | 88.4549 | 3.7638 | 84.6910 |
| B | 473 | ASP | 47.2351 | 6.8458 | 40.3893 |
| B | 474 | ILE | 29.1817 | 24.1296 | 5.0520 |
| B | 475 | SER | 14.3571 | 4.1523 | 10.2049 |
| B | 476 | VAL | 20.6222 | 18.5324 | 2.0898 |
| B | 477 | GLN | 10.6533 | 0.0000 | 10.6533 |
| B | 478 | TRP | 1.8912 | 1.0361 | 0.8551 |
| B | 479 | LEU | 28.2585 | 0.9455 | 27.3131 |
| B | 480 | HIS | 9.7124 | 1.2163 | 8.4961 |
| B | 481 | ASN | 54.6134 | 14.8060 | 39.8074 |
| B | 482 | GLU | 172.9182 | 41.2890 | 131.6292 |
| B | 483 | VAL | 80.4369 | 2.9369 | 77.5000 |
| B | 484 | GLN | 86.7995 | 23.2392 | 63.5604 |
| B | 485 | LEU | 20.1440 | 8.7226 | 11.4214 |
| B | 486 | PRO | 79.3531 | 10.9230 | 68.4300 |
| B | 487 | ASP | 113.7037 | 8.5271 | 105.1766 |
| B | 488 | ALA | 105.1557 | 42.1652 | 62.9905 |
| B | 489 | ARG | 78.5174 | 20.7364 | 57.7810 |
| B | 490 | HIS | 27.7987 | 16.2252 | 11.5735 |
| B | 491 | SER | 30.5621 | 5.9459 | 24.6161 |
| B | 492 | THR | 40.2793 | 15.9582 | 24.3211 |
| B | 493 | THR | 12.2109 | 4.6417 | 7.5692 |
| B | 494 | GLN | 99.8352 | 2.7394 | 97.0958 |
| B | 495 | PRO | 38.5051 | 18.8569 | 19.6482 |
| B | 496 | ARG | 86.5718 | 5.6625 | 80.9093 |
| B | 497 | LYS | 159.5251 | 21.1005 | 138.4247 |
| B | 498 | THR | 19.4014 | 18.8518 | 0.5496 |
| B | 499 | LYS | 201.9754 | 42.6147 | 159.3607 |
| B | 500 | GLY | 44.4883 | 44.4883 | 0.0000 |
| B | 501 | SER | 85.5433 | 25.6861 | 59.8572 |
| B | 502 | GLY | 1.9867 | 1.9867 | 0.0000 |
| B | 503 | PHE | 31.1871 | 0.0000 | 31.1871 |
| B | 504 | PHE | 1.5596 | 0.0598 | 1.4998 |
| B | 505 | VAL | 0.9708 | 0.0000 | 0.9708 |
| B | 506 | PHE | 3.7613 | 0.1850 | 3.5763 |
| B | 507 | SER | 0.6262 | 0.0195 | 0.6068 |
| B | 508 | ARG | 9.5731 | 1.8218 | 7.7513 |
| B | 509 | LEU | 0.7046 | 0.0000 | 0.7046 |
| B | 510 | GLU | 62.9883 | 2.0494 | 60.9388 |
| B | 511 | VAL | 10.8762 | 6.5365 | 4.3397 |
| B | 512 | THR | 71.0071 | 0.0263 | 70.9809 |
| B | 513 | ARG | 104.2393 | 0.0000 | 104.2393 |
| B | 514 | ALA | 65.4206 | 13.1707 | 52.2499 |
| B | 515 | GLU | 35.3933 | 0.3618 | 35.0315 |
| B | 516 | TRP | 39.6550 | 3.3357 | 36.3193 |
| B | 517 | GLU | 115.3847 | 37.2436 | 78.1411 |
| B | 518 | GLN | 113.6505 | 33.6727 | 79.9778 |
| B | 519 | LYS | 84.0921 | 8.7130 | 75.3791 |
| B | 520 | ASP | 64.5129 | 2.3376 | 62.1754 |
| B | 521 | GLU | 87.5584 | 2.3918 | 85.1666 |
| B | 522 | PHE | 9.6600 | 0.0000 | 9.6600 |
| B | 523 | ILE | 30.2820 | 0.0000 | 30.2820 |
| B | 524 | CYS | 0.0000 | 0.0000 | 0.0000 |
| B | 525 | ARG | 37.1783 | 0.0000 | 37.1783 |
| B | 526 | ALA | 0.3818 | 0.3818 | 0.0000 |
| B | 527 | VAL | 0.0418 | 0.0000 | 0.0418 |
| B | 528 | HIS | 0.6191 | 0.1432 | 0.4759 |
| B | 529 | GLU | 49.1816 | 17.5861 | 31.5955 |
| B | 530 | ALA | 25.6926 | 20.8074 | 4.8853 |
| B | 531 | ALA | 7.1284 | 6.1152 | 1.0132 |
| B | 532 | SER | 92.9571 | 24.3505 | 68.6065 |
| B | 533 | PRO | 129.7170 | 30.9019 | 98.8150 |
| B | 534 | SER | 65.6135 | 12.1706 | 53.4428 |
| B | 535 | GLN | 60.5061 | 0.0227 | 60.4835 |
| B | 536 | THR | 22.1684 | 5.5560 | 16.6125 |
| B | 537 | VAL | 29.6659 | 6.3253 | 23.3407 |
| B | 538 | GLN | 69.4992 | 13.4096 | 56.0896 |
| B | 539 | ARG | 92.2922 | 3.4257 | 88.8665 |
| B | 540 | ALA | 62.4168 | 19.4066 | 43.0101 |
| B | 541 | VAL | 19.1443 | 12.3199 | 6.8244 |
| B | 542 | SER | 49.6556 | 19.3884 | 30.2672 |
| B | 543 | VAL | 20.6069 | 2.0847 | 18.5222 |
| B | 544 | ASN | 178.7782 | 70.1438 | 108.6343 |
| B | 694 | NAG | 107.4774 | 0.0000 | 107.4774 |
| B | 695 | NAG | 119.4719 | 0.0000 | 119.4719 |
| B | 696 | MAN | 45.7067 | 0.0000 | 45.7067 |
| B | 697 | MAN | 152.8463 | 0.0000 | 152.8463 |
| B | 698 | MAN | 222.3243 | 0.0000 | 222.3243 |
| B | 699 | MAN | 217.3122 | 0.0000 | 217.3122 |
| D | 329 | CYS | 102.3809 | 67.5332 | 34.8476 |
| D | 330 | ASP | 111.3542 | 32.4992 | 78.8550 |
| D | 331 | SER | 49.5069 | 8.1508 | 41.3561 |
| D | 332 | ASN | 19.9483 | 7.1538 | 12.7945 |
| D | 333 | PRO | 20.7718 | 9.3148 | 11.4570 |
| D | 334 | ARG | 103.0460 | 10.1992 | 92.8468 |
| D | 335 | GLY | 3.3799 | 3.3799 | 0.0000 |
| D | 336 | VAL | 12.9305 | 10.8477 | 2.0827 |
| D | 337 | SER | 17.9779 | 5.3981 | 12.5798 |
| D | 338 | ALA | 2.9541 | 2.7192 | 0.2349 |
| D | 339 | TYR | 68.8053 | 3.1433 | 65.6620 |
| D | 340 | LEU | 28.1176 | 26.0926 | 2.0250 |
| D | 341 | SER | 59.6285 | 9.0359 | 50.5926 |
| D | 342 | ARG | 87.3522 | 11.5821 | 75.7702 |
| D | 343 | PRO | 7.5238 | 7.5238 | 0.0000 |
| D | 344 | SER | 36.9529 | 6.5208 | 30.4322 |
| D | 345 | PRO | 11.5386 | 3.1229 | 8.4157 |
| D | 346 | PHE | 45.5005 | 0.0000 | 45.5005 |
| D | 347 | ASP | 47.0584 | 0.0024 | 47.0561 |
| D | 348 | LEU | 9.0480 | 0.3353 | 8.7126 |
| D | 349 | PHE | 22.9831 | 9.2421 | 13.7410 |
| D | 350 | ILE | 57.3966 | 13.5038 | 43.8927 |
| D | 351 | ARG | 140.1074 | 22.9695 | 117.1379 |
| D | 352 | LYS | 139.7937 | 14.7242 | 125.0695 |
| D | 353 | SER | 48.1517 | 7.3618 | 40.7899 |
| D | 354 | PRO | 3.2206 | 0.8205 | 2.4000 |
| D | 355 | THR | 54.2972 | 11.4293 | 42.8679 |
| D | 356 | ILE | 0.4144 | 0.4144 | 0.0000 |
| D | 357 | THR | 39.9578 | 1.7071 | 38.2507 |
| D | 358 | CYS | 0.3097 | 0.0000 | 0.3097 |
| D | 359 | LEU | 18.5271 | 0.0018 | 18.5253 |
| D | 360 | VAL | 1.3679 | 0.0000 | 1.3679 |
| D | 361 | VAL | 0.3469 | 0.0000 | 0.3469 |
| D | 362 | ASP | 12.4469 | 6.3831 | 6.0638 |
| D | 363 | LEU | 4.9868 | 1.6440 | 3.3428 |
| D | 364 | ALA | 19.9780 | 5.4292 | 14.5488 |
| D | 365 | PRO | 65.0465 | 6.1738 | 58.8727 |
| D | 366 | SER | 32.8048 | 26.7953 | 6.0096 |
| D | 367 | LYS | 136.0098 | 36.9049 | 99.1049 |
| D | 368 | GLY | 26.7169 | 26.7169 | 0.0000 |
| D | 369 | THR | 93.8010 | 14.7194 | 79.0816 |
| D | 370 | VAL | 15.1817 | 1.2150 | 13.9667 |
| D | 371 | ASN | 71.4877 | 1.7213 | 69.7664 |
| D | 372 | LEU | 27.5581 | 21.2832 | 6.2749 |
| D | 373 | THR | 64.9412 | 3.5271 | 61.4141 |
| D | 374 | TRP | 21.6411 | 12.7579 | 8.8832 |
| D | 375 | SER | 45.5983 | 4.0264 | 41.5719 |
| D | 376 | ARG | 46.1407 | 18.3951 | 27.7456 |
| D | 377 | ALA | 73.2052 | 42.7392 | 30.4661 |
| D | 378 | SER | 60.9391 | 42.2524 | 18.6867 |
| D | 379 | GLY | 62.4810 | 62.4810 | 0.0000 |
| D | 380 | LYS | 114.3210 | 4.9018 | 109.4192 |
| D | 381 | PRO | 118.6128 | 19.3560 | 99.2568 |
| D | 382 | VAL | 56.2105 | 27.7078 | 28.5027 |
| D | 383 | ASN | 89.7140 | 9.4333 | 80.2807 |
| D | 384 | HIS | 175.3907 | 15.2111 | 160.1796 |
| D | 385 | SER | 60.1172 | 34.0544 | 26.0628 |
| D | 386 | THR | 63.3471 | 6.6705 | 56.6765 |
| D | 387 | ARG | 100.5610 | 32.1288 | 68.4323 |
| D | 388 | LYS | 117.6302 | 8.3122 | 109.3179 |
| D | 389 | GLU | 93.9137 | 30.6429 | 63.2707 |
| D | 390 | GLU | 96.2332 | 5.9265 | 90.3067 |
| D | 391 | LYS | 170.3439 | 24.4483 | 145.8956 |
| D | 392 | GLN | 31.5360 | 6.5112 | 25.0248 |
| D | 393 | ARG | 229.7092 | 34.9960 | 194.7132 |
| D | 394 | ASN | 71.2822 | 36.9290 | 34.3532 |
| D | 395 | GLY | 54.2216 | 54.2216 | 0.0000 |
| D | 396 | THR | 14.4859 | 6.7168 | 7.7691 |
| D | 397 | LEU | 31.2188 | 0.0000 | 31.2188 |
| D | 398 | THR | 0.9937 | 0.0352 | 0.9584 |
| D | 399 | VAL | 1.8265 | 0.0023 | 1.8242 |
| D | 400 | THR | 28.8418 | 0.0000 | 28.8418 |
| D | 401 | SER | 1.8342 | 0.0000 | 1.8342 |
| D | 402 | THR | 38.4300 | 1.3612 | 37.0688 |
| D | 403 | LEU | 1.3700 | 0.0000 | 1.3700 |
| D | 404 | PRO | 56.4630 | 5.0109 | 51.4521 |
| D | 405 | VAL | 10.3785 | 9.6144 | 0.7642 |
| D | 406 | GLY | 19.9415 | 19.9415 | 0.0000 |
| D | 407 | THR | 17.7730 | 0.0342 | 17.7388 |
| D | 408 | ARG | 149.6237 | 4.1119 | 145.5118 |
| D | 409 | ASP | 52.0866 | 6.4418 | 45.6449 |
| D | 410 | TRP | 13.1899 | 0.0000 | 13.1899 |
| D | 411 | ILE | 63.5723 | 7.3792 | 56.1932 |
| D | 412 | GLU | 148.7782 | 36.0783 | 112.6999 |
| D | 413 | GLY | 38.9396 | 38.9396 | 0.0000 |
| D | 414 | GLU | 20.8518 | 6.7344 | 14.1173 |
| D | 415 | THR | 39.3540 | 0.6913 | 38.6627 |
| D | 416 | TYR | 5.8935 | 0.0014 | 5.8921 |
| D | 417 | GLN | 62.2903 | 0.0009 | 62.2895 |
| D | 418 | CYS | 0.4753 | 0.3779 | 0.0974 |
| D | 419 | ARG | 106.4535 | 0.4475 | 106.0060 |
| D | 420 | VAL | 2.7864 | 0.0595 | 2.7269 |
| D | 421 | THR | 47.3939 | 3.4111 | 43.9828 |
| D | 422 | HIS | 13.1471 | 8.0455 | 5.1016 |
| D | 423 | PRO | 73.2651 | 38.6918 | 34.5733 |
| D | 424 | HIS | 5.0313 | 3.9403 | 1.0910 |
| D | 425 | LEU | 26.2169 | 16.8989 | 9.3180 |
| D | 426 | PRO | 16.7230 | 16.7230 | 0.0000 |
| D | 427 | ARG | 90.3191 | 7.4734 | 82.8457 |
| D | 428 | ALA | 36.2453 | 22.9999 | 13.2454 |
| D | 429 | LEU | 33.6211 | 0.8879 | 32.7333 |
| D | 430 | MET | 81.2915 | 16.0381 | 65.2534 |
| D | 431 | ARG | 95.2832 | 10.2332 | 85.0500 |
| D | 432 | SER | 62.4521 | 22.5286 | 39.9235 |
| D | 433 | THR | 16.4152 | 7.6464 | 8.7688 |
| D | 434 | THR | 43.2290 | 5.7565 | 37.4725 |
| D | 435 | LYS | 48.4737 | 13.5566 | 34.9171 |
| D | 436 | THR | 40.0113 | 7.5864 | 32.4249 |
| D | 437 | SER | 92.1976 | 14.6061 | 77.5915 |
| D | 438 | GLY | 47.1703 | 47.1703 | 0.0000 |
| D | 439 | PRO | 87.5094 | 11.3492 | 76.1603 |
| D | 440 | ARG | 91.1554 | 32.4332 | 58.7222 |
| D | 441 | ALA | 37.6064 | 5.6168 | 31.9896 |
| D | 442 | ALA | 47.7716 | 10.8839 | 36.8877 |
| D | 443 | PRO | 4.7444 | 4.7444 | 0.0000 |
| D | 444 | GLU | 31.7469 | 0.2472 | 31.4997 |
| D | 445 | VAL | 2.2316 | 0.4056 | 1.8260 |
| D | 446 | TYR | 13.0081 | 1.2161 | 11.7920 |
| D | 447 | ALA | 19.3686 | 19.1556 | 0.2130 |
| D | 448 | PHE | 32.4618 | 5.0174 | 27.4445 |
| D | 449 | ALA | 26.4564 | 17.8510 | 8.6054 |
| D | 450 | THR | 6.7460 | 1.4957 | 5.2504 |
| D | 451 | PRO | 78.1205 | 5.0753 | 73.0452 |
| D | 452 | GLU | 98.7545 | 13.9213 | 84.8332 |
| D | 453 | TRP | 95.8047 | 1.7250 | 94.0797 |
| D | 454 | PRO | 125.4561 | 37.0254 | 88.4307 |
| D | 455 | GLY | 65.7398 | 65.7398 | 0.0000 |
| D | 456 | SER | 42.2645 | 8.7091 | 33.5554 |
| D | 457 | ARG | 185.3827 | 19.6535 | 165.7292 |
| D | 458 | ASP | 64.7836 | 1.4527 | 63.3309 |
| D | 459 | LYS | 150.1746 | 13.4092 | 136.7655 |
| D | 460 | ARG | 41.0497 | 3.6090 | 37.4407 |
| D | 461 | THR | 12.7243 | 1.3702 | 11.3540 |
| D | 462 | LEU | 5.8977 | 0.0000 | 5.8977 |
| D | 463 | ALA | 9.3450 | 0.3148 | 9.0302 |
| D | 464 | CYS | 0.6992 | 0.6992 | 0.0000 |
| D | 465 | LEU | 2.3713 | 0.0565 | 2.3149 |
| D | 466 | ILE | 0.3495 | 0.0005 | 0.3490 |
| D | 467 | GLN | 5.5766 | 0.0000 | 5.5766 |
| D | 468 | ASN | 37.3320 | 5.0478 | 32.2843 |
| D | 469 | PHE | 0.0020 | 0.0020 | 0.0000 |
| D | 470 | MET | 31.1122 | 0.0000 | 31.1122 |
| D | 471 | PRO | 7.4404 | 4.3666 | 3.0739 |
| D | 472 | GLU | 83.9660 | 3.9591 | 80.0069 |
| D | 473 | ASP | 40.3144 | 6.4854 | 33.8290 |
| D | 474 | ILE | 29.6267 | 24.7486 | 4.8781 |
| D | 475 | SER | 15.2528 | 4.1160 | 11.1368 |
| D | 476 | VAL | 20.2396 | 18.2215 | 2.0182 |
| D | 477 | GLN | 12.4429 | 0.0027 | 12.4403 |
| D | 478 | TRP | 1.7703 | 0.9849 | 0.7854 |
| D | 479 | LEU | 28.1196 | 1.0451 | 27.0745 |
| D | 480 | HIS | 9.4122 | 1.1137 | 8.2985 |
| D | 481 | ASN | 56.0442 | 16.0061 | 40.0381 |
| D | 482 | GLU | 170.6455 | 41.2302 | 129.4154 |
| D | 483 | VAL | 80.9853 | 2.7860 | 78.1993 |
| D | 484 | GLN | 88.3797 | 22.1725 | 66.2072 |
| D | 485 | LEU | 20.3000 | 9.0697 | 11.2303 |
| D | 486 | PRO | 79.3386 | 10.6028 | 68.7358 |
| D | 487 | ASP | 114.5014 | 8.7729 | 105.7285 |
| D | 488 | ALA | 104.3458 | 41.8161 | 62.5297 |
| D | 489 | ARG | 79.9265 | 20.4064 | 59.5200 |
| D | 490 | HIS | 27.6480 | 16.2092 | 11.4388 |
| D | 491 | SER | 29.4802 | 5.4279 | 24.0523 |
| D | 492 | THR | 40.7927 | 16.2337 | 24.5591 |
| D | 493 | THR | 11.8506 | 4.3668 | 7.4838 |
| D | 494 | GLN | 119.5958 | 2.8589 | 116.7369 |
| D | 495 | PRO | 39.1911 | 18.6929 | 20.4981 |
| D | 496 | ARG | 86.9475 | 5.7213 | 81.2261 |
| D | 497 | LYS | 160.3208 | 19.6386 | 140.6822 |
| D | 498 | THR | 18.7636 | 18.4350 | 0.3286 |
| D | 499 | LYS | 200.0818 | 42.2517 | 157.8301 |
| D | 500 | GLY | 44.5668 | 44.5668 | 0.0000 |
| D | 501 | SER | 86.6338 | 25.9131 | 60.7207 |
| D | 502 | GLY | 1.8767 | 1.8767 | 0.0000 |
| D | 503 | PHE | 31.3369 | 0.0034 | 31.3334 |
| D | 504 | PHE | 1.4032 | 0.0633 | 1.3399 |
| D | 505 | VAL | 0.8780 | 0.0000 | 0.8780 |
| D | 506 | PHE | 4.3508 | 0.1793 | 4.1714 |
| D | 507 | SER | 0.5298 | 0.0000 | 0.5298 |
| D | 508 | ARG | 8.9714 | 1.8540 | 7.1174 |
| D | 509 | LEU | 0.7079 | 0.0000 | 0.7079 |
| D | 510 | GLU | 61.8196 | 1.9056 | 59.9140 |
| D | 511 | VAL | 10.8929 | 6.6139 | 4.2790 |
| D | 512 | THR | 71.8291 | 0.0838 | 71.7453 |
| D | 513 | ARG | 105.1744 | 0.0000 | 105.1744 |
| D | 514 | ALA | 65.9787 | 13.5900 | 52.3887 |
| D | 515 | GLU | 36.2623 | 0.0481 | 36.2142 |
| D | 516 | TRP | 40.1069 | 3.3758 | 36.7311 |
| D | 517 | GLU | 115.7130 | 37.9922 | 77.7208 |
| D | 518 | GLN | 110.4985 | 32.2394 | 78.2591 |
| D | 519 | LYS | 84.2805 | 8.6356 | 75.6448 |
| D | 520 | ASP | 65.1989 | 2.2319 | 62.9670 |
| D | 521 | GLU | 87.5054 | 1.9237 | 85.5818 |
| D | 522 | PHE | 9.4572 | 0.0000 | 9.4572 |
| D | 523 | ILE | 31.1442 | 0.0000 | 31.1442 |
| D | 524 | CYS | 0.0003 | 0.0003 | 0.0000 |
| D | 525 | ARG | 39.5175 | 0.0000 | 39.5175 |
| D | 526 | ALA | 0.4314 | 0.4314 | 0.0000 |
| D | 527 | VAL | 0.1473 | 0.0000 | 0.1473 |
| D | 528 | HIS | 0.6558 | 0.1273 | 0.5285 |
| D | 529 | GLU | 49.3913 | 17.1907 | 32.2006 |
| D | 530 | ALA | 25.8310 | 20.8648 | 4.9662 |
| D | 531 | ALA | 6.3430 | 5.3376 | 1.0054 |
| D | 532 | SER | 90.9851 | 24.3589 | 66.6262 |
| D | 533 | PRO | 129.9180 | 31.2948 | 98.6232 |
| D | 534 | SER | 63.8534 | 10.9652 | 52.8881 |
| D | 535 | GLN | 62.1692 | 0.1376 | 62.0316 |
| D | 536 | THR | 20.7628 | 5.8645 | 14.8983 |
| D | 537 | VAL | 29.9134 | 6.1442 | 23.7692 |
| D | 538 | GLN | 73.2944 | 13.0975 | 60.1969 |
| D | 539 | ARG | 94.0071 | 3.3512 | 90.6559 |
| D | 540 | ALA | 62.7499 | 19.2166 | 43.5333 |
| D | 541 | VAL | 20.0580 | 13.1091 | 6.9490 |
| D | 542 | SER | 52.5909 | 21.4389 | 31.1520 |
| D | 543 | VAL | 20.1018 | 2.8350 | 17.2667 |
| D | 544 | ASN | 177.6491 | 71.3693 | 106.2798 |
| D | 694 | NAG | 136.0235 | 0.0000 | 136.0235 |
| D | 695 | NAG | 128.7899 | 0.0000 | 128.7899 |
| D | 696 | MAN | 176.7398 | 0.0000 | 176.7398 |
| E | 101 | CPS | 163.2849 | 0.0000 | 163.2849 |
| E | 102 | CHA | 333.1883 | 0.0000 | 333.1883 |
| E | 103 | CPS | 83.0589 | 0.0000 | 83.0589 |
| E | 104 | CPS | 313.3217 | 0.0000 | 313.3217 |
| E | 105 | CPS | 246.4972 | 0.0000 | 246.4972 |

Residues that are solvent accessible are important as they represent amino acids that are on the external surface of the proteins in the complex and, as such, may be involved in binding of a FcR to an antibody and as such be useful in designing proteins with an enhanced binding activity or in identifying compounds that inhibit such binding. In addition, solvent accessible residues can represent targets for modification to produce a FcR or antibody with improved function. Such analysis also identifies residues in the interior, or core, of the proteins in the complex. Such residues can also be targeted to produce proteins with improved functions, such as enhanced stability.

A model of the present invention also provides additional information that is not available from other sources. For example, a model can identify the crystal contacts between crystals and predict the location of the IgE binding domain, including those amino acids that actually form contacts with a Fc domain of an IgE antibody, such as those in the binding face of the FcεRIα protein. A model can also identify the amino acids in the interface between domain 1 and domain 2 (i.e., the D1D2 interface), as well as those in the cleft formed between the two domains of the FcεRIα protein. Particularly important regions of the complex indicated by the model represented in Table 1 include, but are not limited to, FcεRIα:Fc-Cε3/Cε4 interaction site 1, FcεRIα:Fc-Cε3/Cε4 interaction site 2, the hinge between domain Cε3 and domain Cε4 of the Fc-Cε3/Cε4 region, and a FcERIa:Fc-Cε3/Cε4 region that interacts with 3-[3-(cholamidopropyl) dimethylammonio]-1-propane-sulfonate (CHAPS). Interaction sites 1 and 2 are the sites at which amino acids from FcεRIα and Fc-Cε3/Cε4 interact with each other. These sites are described in more detail in the Examples and represent sites to target for drug design and mutein production.

One embodiment of the present invention is a model that represents a complex that includes a protein that binds to a Fc domain of an IgE antibody with an affinity that is at least equivalent to the affinity of the extracellular domain of human FcεRIα for any one of the following IgE antibodies: a human IgE antibody, a canine IgE antibody, a feline IgE antibody, an equine IgE antibody, a rat IgE antibody, and a murine IgE antibody. Such a model can represent an extracellular domain of a human FcεRIα protein, a canine FcεRIα protein, a feline FcεRIα protein, an equine FcεRIα protein, a murine FcεRIα protein, and a rat FcεRIα protein. Such a model can also represent a protein with altered substrate specificity, preferably designed based on a model of the present invention. WO 98/23964, ibid., reports the ability of an isolated human FcαRIα protein to bind to canine, feline and equine IgE antibodies. Models of the present invention can be used to design a FcR with increased affinity for an antibody of a species other than self, such as, but not limited to, a human FcεRIα with increased affinity for a canine, feline or equine IgE antibody.

A model of the present invention can also represent a complex that includes a Fc domain of an antibody that binds to a FcεRIα protein with an affinity that is at least equivalent to the affinity of a human IgE antibody Fc-Cε3/Cε4 region for the extracellular domain of any of the following FcεRIα proteins: a human FcεRIα protein, a canine FcεRIα protein, a feline FcεRIα protein, an equine FcαRIα protein, a murine FcεRIα protein and a rat FcεRIα protein. Such a model can represent a FceRI-binding domain of a human, canine, feline, equine, murine or rat Fe region. Such a model can also represent a Fe region with altered substrate specificity, preferably designed based on a model of the present invention.

The present invention includes a model that represents a complex between a FcR and a Fe domain that binds to an antibody or receptor of its respective class (i.e., IgE, IgG, IgM, IgA or IgD antibody class or corresponding Fe receptor). Also included is a model that represents a complex between a FcR and antibody designed to bind to an antibody or receptor, respectively, of a class other than the class to which the protein naturally binds. Such a model of the present invention can be produced, for example, by incorporating all or any part of the amino acid sequence of the other FcR or antibody into a 3-D model substantially representing the coordinates in Table 1. Such an embodiment includes any model that specifically incorporates any Ig domains that are placed in an orientation (packing interfaces and bend angles) that is based on the structure of the FcεRI or a model that is based on the 1:1 stoichiometry predicted by the coordinates in Table 1. A preferred model of the present invention represents a complex including a FcR that binds to an IgE antibody or to an IgG antibody. In one embodiment, a model of the present invention is a 3-D model of a complex between an extracellular antibody binding domain of a FcR other than human FcεRIα, such as of a FcR that binds to an IgG antibody and an antibody. Such proteins and models thereof can be designed by homology modeling by, for example, altering the substrate specificity of a FcεRIα protein such that the altered protein binds an IgG antibody.

A preferred modified model of the present invention is a model that has a 3-D structure comprising atomic coordinates that have a root mean square deviation of protein backbone atoms of less than 10 angstrom when superimposed, using backbone atoms, on the 3-D model substantially represented by the atomic coordinates specified in Table 1. Preferably such a model has a 3-D structure comprising atomic coordinates that have a root mean square deviation of protein backbone atoms of less than 8 angstroms, preferably less than 7 angstroms, preferably less than 6 angstroms, preferably less than 5 angstroms, preferably less than 4 angstroms, preferably less than 3 angstroms, preferably less than 2 angstroms, and preferably less than 1 angstroms, when superimposed, using backbon3 atoms, on the 3-D model substantially represented by the atomic coordinates specified in Table 1. In this embodiment, such a model represents a FcR binding to an antibody. The backbone atoms are those atoms that form the backbone, or 3-D folding pattern, of the model. As such, backbone atoms are the base residues of amino acids, i.e., nitrogen, carbon, the alpha carbon and oxygen. Also preferred is a model modification that includes (a) a FcR protein having an amino acid sequence that shares at least about 30%, preferably at least about 40%, more preferably at least about 45%, more preferably at least about 50%, more preferably at least about 60% and even more preferably at least about 80% amino acid sequence homology, with a human FcεRIα protein, as determined using the program ALIGN with default parameters, optimal global alignment of two sequences with no short-cuts and (b) a Fc region having an amino acid sequence that shares at least about 30%, preferably at least about 40%, more preferably at least about 45%, more preferably at least about 50%, more preferably at least about 60% and even more preferably at least about 80% amino acid sequence homology, with a Fc-Cε3/Cε4 region of a human IgE antibody, as determined using the program ALIGN with default parameters, optimal global alignment of two sequences with no short-cuts. It is to be noted that, using the same program and parameters, the extracellular domain of a human FcεRIα protein (i.e., soluble human FcεRIα protein) shares about 48% identity with feline and rat soluble FcεRIα proteins, about 49% with a murine soluble FcεRIα protein, about 50% identity with a canine soluble FcεRIα protein, and about 60% identity with an equine soluble FcεRIα protein. A preferred model of the present invention represents an IgE binding domain, i.e., a region that binds to an IgE antibody, complexed to a FcεRIα-binding domain, i.e., a region that binds to a FcεRIα protein.

One embodiment of the present invention is a 3-D model of a complex between a human FcεRIα protein and a human Fc-Cε3/Cε4 region produced by a method that includes the steps of: (a) crystallizing a complex between an extracellular domain of a human FcεRIα protein, such as, but not limited to a protein having amino acid sequence SEQ ID NO:2 or SEQ ID NO:4 and a human Fc-Cε3/Cε4 region, such as, but not limited to a protein having amino acid sequence SEQ ID NO:6; (b) collecting X-ray diffraction data from the crystallized complex; and (c) determining the model from the X-ray diffraction data, preferably in combination with an amino acid sequence of the proteins in the complex. A complex for crystal formation can be produced using a variety of techniques well known to those skilled in the art. As disclosed herein, human FcεRIα proteins and human Fc-Cε3/Cε4 region sto be crystallized are preferably produced in recombinant insect cells transformed with a gene encoding the respective proteins, such as a baculovirus genetically engineered to produce the respective protein. The purity of the FcεRIα protein or Fc-Cε3/Cε4 region must be sufficient to permit the production of crystals that can be analyzed by X-ray crystallography to a resolution that permits determination of a 3-D model of the protein. Preferably the resolution is at least about 4.5 angstroms (i.e., 4.4 angstroms or better), more preferably at least about 4 angstroms, more preferably at least about 3.5 angstroms, more preferably at least about 3.25 angstroms, more preferably at least about 3 angstroms, more preferably at least about 2.5 angstroms, more preferably at least about 2 angstroms and even more preferably at least about 1.5 angstroms. Methods to obtain such purity levels are well known to those skilled in the art.

As disclosed herein, a preferred method to crystallize a complex between a FcεRIα protein and a Fc-Cε3/Cε4 region is by vapor distillation. Particularly preferred methods are disclosed in the Examples. It should be appreciated that the present invention also includes other methods known to those skilled in the art by which such a complex can be crystallized.

3-D models of some proteins have been determined; see, for example, Blundell et al., Protein Crystallography, Academic Press, London, 1976. However, as discussed herein, elucidation of the crystal structure of a complex between the extracellular domain of the human FcεRIα and a Fc-Cε3/Cε4 region of a human IgE was difficult. In one embodiment, crystal structure determination includes obtaining high-resolution data using synchrotron radiation. Such data can be collected, for example, at the Stanford Synchrotron Source Laboratory, Palo Alto, Calif., or the Advanced Photon Source at Argonne National Laboratories, Argonne, Ill. Additional locations to collect such data include, but are not limited to, Brookhaven, N.Y., and Japan. In one embodiment, diffraction data from native and heavy-atom treated crystals provide an initial image of the protein structure which is refined into an electron density map. Details regarding data collection and interpretation are provided in the Examples section.

One embodiment of the present invention is a method to produce a 3-D model of a FcεRIα protein that includes positioning amino acid representations (i.e., representing amino acids) of the protein at substantially the coordinates listed in Table 1. That is, knowledge of the coordinates of the complex permits one skilled in the art to produce a model of the complex using those coordinates. Such a model, or any model which is essentially represented by a simple coordinate transformation of the coordinates specified in Table 1, can be represented in a variety of methods as heretofore disclosed and is included in the present invention.

In another embodiment, a model of the present invention can be refined to obtain an improved model, which is an example of a model modification, also referred to as a modified model. Refining methods can include, but are not limited to, further data collection and analysis; data collection from frozen crystals; introduction of solvent molecules to the structure; clarification of secondary structure; and analyses of crystallized complexes between a FcR and an antibody or inhibitory compound or of crystallized FcRs or antibodies alone. An additional model refinement method includes analyzing a 3-D model to predict amino acid residues that if replaced are likely to yield proteins with at least one improved function, effecting at least one such replacement, determining whether the activity of the modified protein agrees with the prediction, and refining the model as necessary. Methods to determine whether the modification agrees with prediction include producing the modified protein and performing assays with that modified protein to determine if the protein does indeed exhibit the improved function(s), such as desired activity, stability and solubility properties. Assays to measure such functions are well known in the art; examples of several such assays are disclosed herein.

Another embodiment of the present invention is a modified 3-D model that represents a complex between a FcR other than a human FcεRIα protein represented by the 3-D model the coordinates of which are listed in Table 1 and an antibody other than human IgE as represented by the coordinates in Table 1. Preferably the amino acid sequence of the protein(s) to be modeled is known. In such a case, the modified model can be produced using the technique of homology modeling, preferably by incorporating (e.g., grafting, overlaying or replacing) all or any portion of the amino acid sequence of the other FcR or antibody into the 3-D model representing the coordinates of Table 1 to produce the modified model. General techniques for homology modeling, also referred to as molecular replacement, have been disclosed in, for example, Greer, 1990, Proteins: Structure, Function, and Genetics 7, 317-334; Havel et al., 1991, J. Mol. Biol. 217, 1-7; Schiffer et al., 1990, Proteins: Structure, Function, and Genetics 8, 30-43; and Lattman, 1985, Methods Enzymol 115, 55-77. However, such technology has not been applied to complexes between FcRs and antibodies since, until the present invention, no 3-D model of any FcR:antibody complex was available. Thus, the present invention now allows the solving of the structures of a number of other natural and mutated forms of FcRs, antibodies or complexes thereof.

In one embodiment, a model of a FcR:Fc complex, such as, but not limited to a FcεRIα:Fc-Cε3/Cε4 complex, is produced by extracting the 3-D coordinates from a published figure or building a 3-D model with atoms from other domains wherein the domain 1 and 2 of the FcR and FcR-binding domains of the antibody are oriented as predicted for a complex between the human FcεRIα₁₋₁₇₆ protein and human Fc-Cε3/Cε4₂₂₂ protein. For example, a model of the present invention can be produced by orienting two known Ig domains into a bent confirmation similar to that of the two domains of the human FcεRIα protein. Such a model is referred to as a model in which domain 1 and domain 2 are oriented in a manner as specified by the structural coordinates listed in Table 1. This model can then be used in further molecular replacement methods. Such methods can include the steps of (a) orienting the model by three rotations; and (b) translating the model in one to three directions to produce additional model modifications.

Suitable FcRs or antibodies for which a 3-D model can be determined using homology modeling include any mammalian FcR or antibody, such as a protein that binds to IgE, IgG, IgM, IgA or IgD antibodies or an antibody that binds to the corresponding FcR. Preferred is a FcR protein that binds to an IgE antibody or an IgG antibody. Preferred FcRs that bind to IgE include human, canine, feline, equine, murine and rat FcεRIα proteins. Preferred antibodies that bind to FcRs include human, canine, feline, equine, murine and rat antibodies. The present invention also includes the use of other Ig domains to produce models of the present invention.

One embodiment of the present invention is a 3-D model of a FcR:antibody complex in which one or both proteins have an improved function compared to an unmodified protein as well as a method to produce such a modified model. Such an improved function includes, but is not limited to, enhanced activity, enhanced stability and enhanced solubility. Such a modified model can be produced by replacing at least one amino acid based on information derived from analyzing the 3-D model representing the coordinates in Table 1, such that the replacement leads to a protein with an improved function. As used herein, a replacement refers to an (i.e., one or more) amino acid substitution, insertion, deletion, inversion and/or derivatization (e.g., acetylation, glycosylation, phosphorylation, PEG modification, biotinylation, and covalent attachment of other ligands or other compounds to the protein. In one embodiment, synthetic chemical methods are used to produce either a fragment or the entire protein to, for example, introduce non-natural amino acids or other chemical compounds into the structure of a FcR or antibody. For example, based on a structure of the present invention, one can design synthetic peptides or larger proteins that could be linked to produce an intact protein with IgE or FcR binding activity, the structure allowing one to design the start and stop points for these peptides, e.g., at surface accessible loops. In accordance with the present invention, an amino acid that is substituted or inserted can be a natural amino acid or an unnatural amino acid, including a derivitized amino acid. Methods to identify regions in the protein that, if changed, yield a protein with an improved function are disclosed below.

The present invention includes use of a 3-D model of the present invention to identify a compound that inhibits binding between a FcR and an antibody. The advantages of using a 3-D model to identify inhibitory compounds are multi-fold in that the model depicts the site at which a Fc region of an antibody binds to its FcR, i.e., the antibody-binding domain, also referred to as the antibody binding site, and the FcR-binding domain, also referred to as the FcR binding site. The antibody binding site and the FcR binding site together form an FcR:antibody interaction site. As such, a large number of potential inhibitory compounds can be initially analyzed without having to perform in vitro or in vivo laboratory studies. As used herein, methods to identify inhibitory compounds include, but are not limited to, designing inhibitory compounds based on the 3-D model of a FcR, probing such a 3-D model with compounds that are potential inhibitors in order to identify those compounds that are actually inhibitory of the binding of an antibody to its FcR, screening a compound data base using such a 3-D model to identify compounds that inhibit such binding, and combinations thereof. Methods to use 3-D models to design, probe for, or screen for suitable inhibitory compounds are known to those skilled in the art. In particular, there are a number of computer programs that enable such methods. See, for example, PCT Publication No. WO 95/35367, by Wilson et al., published Dec. 28, 1995, which is incorporated by reference herein in its entirety.

An inhibitory compound can be any natural or synthetic compound that inhibits the binding of an antibody to a FcR. Examples include, but are not limited to, inorganic compounds, oligonucleotides, proteins, peptides, antibodies, antibody fragments, mimetics of peptides or antibodies (such as, mimetics of antibody or receptor binding sites), and other organic compounds. Compounds can inhibit binding in either a competitive or non-competitive manner and can either interact at the binding site or allosterically. An inhibitory compound should be capable of physically and structurally associating with a FcR and/or an antibody such that the compound can inhibit binding between the two entitites. As such, an inhibitory compound is preferably small and is of a structure that effectively prevents or disrupts binding. Inhibitory compounds can be identified in one or multiple steps. For example, a compound initially identified that inhibits binding between an antibody and FcR to some extent can be used as a lead to design, probe or screen for a compound with improved characteristics, such as greater efficacy, safety, solubility, etc. A preferred inhibitory compound is a compound that is efficacious when administered to an animal in an amount that results in a serum concentration of from about 1 nanomolar (nM) to 100 micromolar (mM), with a concentration of from about 10 nM to 10 mM being more preferred.

One embodiment of the present invention is a method to identify a compound that inhibits the binding between an IgE antibody and a FcεRIα protein. Such a method includes the step of using a 3-D model substantially representing the atomic coordinates specified in Table 1 to identify such a compound. Included in the present invention are inhibitory compounds that interact directly with the IgE binding domain or the receptor binding domain of the IgE antibody as well as compounds that interact indirectly with an FcεRIα protein, such as compounds that interact with the IgE binding domain, the FcεRIα binding domain, FcεRIα:Fc-Cε3/Cε4 interaction site 1, FcεRIα:Fc-Cε3/Cε4 interaction site 2, the hinge between domain Cε3 and domain Cε4 of the Fc-Cε3/Cε4 region, or a FcεRIα:Fc-Cε3/Cε4 region that interacts with CHAPS. In a preferred embodiment, an inhibitory compound interacts with at least one of the following regions of a model representing a FcεRIα:Fc-Cε3/Cε4 complex: a C strand of domain 2 of FcεRIα, a C′E loop of domain 2 of FcεRIα, a tryptophan-containing hydrophobic ridgeof FcεRIα, a linker between domain 1 and domain 2 of FcεRIα, a BC loop of domain 2 of FcεRIα, a FG loop of domain 2 of FcεRIα, a Cε2/Cε3 linker region of Fc-Cε3/Cε4, a BC loop of Fc-Cε3/Cε4, a DE loop of Fc-Cε3/Cε4, and a FG loop of Fc-Cε3/Cε4. Inhibitory compounds of the present invention preferably interact with at least one of the following amino acids: (a) a residue having a position in SEQ ID NO:2 selected from the group consisting of position 85, 86, 87, 110, 113, 117, 119, 126, 129, 130, 131, 132, 156, 157, and 158; (b) a residue having a position in SEQ ID NO:6 selected from the group consisting of position 4, 7, 8, 9, 10, 11, 37, 38, 39, 68, 69, 70, 99, 100, 101 and 102; and (c) a surface residue within about 10 angstroms of any of the residues listed in (a) or (b). Particularly preferred amino acids with which to interact are: (a) a residue within the FcεRIα pocket for the proline at position 101 of SEQ ID NO:6, such residues including, but not limited to positions 85, 86, 87 and 110 of SEQ ID NO:2; (b) a residue within the IgE pocket for the tyrosine at position 131 of SEQ ID NO:2, such residues including, but not limited to, positions 9, 11, 37, 39, and 99 of SEQ ID NO:6; and (c) a surface residue within about 10 angstroms of any of said residues of (a) or (b). It is to be noted that the ability to identify such key regions and residues is only possible in view of a model of the present invention. These regions and residues are a refinement of those identified using a FcεRIα model as described in 09/434,193, ibid. or WO 00/26246, ibid. In one embodiment, an inhibitory compound of the present invention is a peptide corresponding to at least a portion of any of the identified regions or a derivative thereof, such as a peptide mimetic or other compound that mimics that peptide.

One embodiment of a method to identify a compound that inhibits the binding between an IgE antibody and a FcεRIα protein includes the steps of: (a) generating a model substantially representing the atomic coordinates listed in Table 1 or of the binding domains thereof, on a computer screen; (b) generating the spacial structure of a compound to be tested; and (c) testing to determine if the compound interacts with said IgE binding domain or FcR binding domain, wherein such an interaction indicates that the compound is capable of inhibiting the binding of an IgE antibody to a FcεRIα protein. In a preferred embodiment, step (a) includes the step of identifying one or more amino acid(s) in the IgE binding domain of FcR binding domain of the model that interact directly with the corresponding domain. Preferably a compound to be tested will interact directly with one or more of those amino acid(s). Preferred amino acids with which an inhibitory compound should interact are disclosed herein.

The present invention also includes inhibitory compounds isolated in accordance with the methods disclosed herein. Methods to produce such compounds in quantities sufficient for use, for example, as protective agents (e.g., preventatives or therapeutics) are known to those skilled in the art. It should also be appreciated that it is within the scope of the present invention to expand the use of models of the present invention to produce models of any suitable FcRs (i.e., model modifications) and to identify compounds that inhibit the binding of antibodies to such FcRs.

A preferred inhibitory compound of the present invention, or lead that can be used to produce a more efficacious inhibitory compound, is a saturated tetracyclic hydrocarbon perhydrocyclopentanophenanthrene or a derivative thereof. Such a compound can include a structure having the following formula:

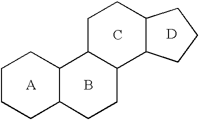

It is to be understood that such a compound can have any number of “R” groups, even though they are not indicated in the formula. Examples of saturated tetracyclic hydrocarbon perhydrocyclopentanophenanthrenes include, but are not limited to, isoprenoids, terpenes, bile acids, detergents (such as CHAPS and CHAPSO) cholestanes, cholic acids, cholesterols, androgens, estrogens, and other steroids. A preferred inhibitory compound, or compound to use as a lead to design a more efficacious compound is 3-[3-(cholamidopropyl) dimethylammonio]-1-propane-sulfonate (CHAPS) or a compound having a similar ring structure. The interaction of CHAPS with amino acids in the FcεRIα protein and Fc-C3/C4 region is described in further detail in the Examples.

The present invention also includes use of a 3-D model of the present invention to rationally design and construct modified forms of FcRs or antibodies that have one or more improved functions, such as, but not limited to, increased activity, increased stability and increased solubility compared to an unmodified FcR or antibody. Muteins of the present invention include full-length proteins as well as fragments (i.e., truncated versions) of such proteins.

One embodiment of the present invention is a FcR that comprises a mutein that binds to a Fc domain of an antibody. Such a mutein has an improved function compared to a protein comprising SEQ ID NO:2. Examples of such an improved function include, but are not limited to, increased stability, increased affinity for an Fc domain of an antibody, altered substrate specificity, and increased solubility. Such a mutein can be produced by a method that includes the steps of: (a) analyzing a 3-D model substantially representing the atomic coordinates specified in Table 1 to identify at least one amino acid of the protein represented by the model which if replaced by a specified amino acid would effect the improved function of the protein; and (b) replacing the identified amino acid(s) to produce a mutein having the improved function. Knowledge of the coordinates allows one to target specific residues, e.g. in the hydrophobic core or on the surface, to generate an accessible set of variants that can then be selected for a particular property, e.g. high stability, high affinity, altered substrate specificity, or other desirable properties (i.e., improved functions). Without the coordinates, one would have to analyze an extraordinarily large number of variants, e.g., on the order of ˜10¹¹ possibilities. The structure, in contrast, allows one to pick the most relevant residues for selecting a desired property by, for example, phage display or other methods. In a preferred embodiment, replacement of one or more amino acids does not substantially disrupt the 3-D structure of the protein; i.e., the modified protein, or mutein, is still capable of binding to the Fc domain of an antibody. A preferred mutein is a FcR that binds to a Fe domain of an IgE antibody, although the invention also covers muteins binding to other classes of antibodies.

In one embodiment, a mutein of the present invention has increased stability compared to its unmodified counterpart. As used herein, increased stability refers to the ability of a mutein to be more resistant, for example, to higher or lower temperature, to more acidic or basic pH, to higher or lower salt concentrations, to oxidation and/or reduction, to deamidation, to other forms of chemical degradation and to proteolytic degradation compared to unmodified FcR. Increased stability can also refer to the ability of a mutein of the present invention to be stable for a longer period of time either during storage (i.e., to have a longer shelf life) or during use (i.e., to have a longer half-life under reaction conditions) than does an unmodified protein. Muteins of the present invention can also exhibit a decreased entropy of unfolding, thereby stabilizing the proteins. Increased stability can be measured using a variety of methods known to those skilled in the art; examples include, but are not limited to, determination of melting temperature, thermal denaturation, pressure denaturation, enthalpy of unfolding, free energy of the protein, or stability in the presence of a chaotropic agents such as urea, guanidinium chloride, guanidinium thiocyanate, etc. A preferred mutein of the present invention has a melting temperature substantially higher than that of an unmodified FcR. Preferably the melting temperature of a mutein is at least about 1° C. higher, and more preferably at least about 10° C. higher than the melting temperature of the corresponding unmodified protein. Also preferred is a mutein having binding activity over a pH range that is at least about 1 pH unit higher and/or lower than the active pH range of the corresponding unmodified protein.

Another embodiment of the present invention is a mutein that exhibits increased affinity for a Fc domain of an antibody compared to its unmodified counterpart. As used herein, a mutein having increased affinity is a FcR that exhibits a higher affinity constant (K_{A}) or lower dissociation constant (K_{D}) than its unmodified counterpart. Such a higher affinity constant can be achieved by increasing the association rate (kₐ) between the mutein and the Fc domain and/or decreasing the dissociation rate (k_{d}) between the mutein and the Fc domain. A preferred mutein of the present invention has a K_{A} for a Fc domain of at least about 3×10⁹ liters/mole (M⁻¹), which is equivalent to a K_{D} of less than or equal to about 3.3×10⁻¹⁰ moles/liter (M). More preferred is a mutein having a K_{A} for a Fc domain of at least about 2×10¹⁰ M⁻¹, and even more preferably of at least about 1×10¹¹ M⁻¹. Also preferred is a mutein having a kₐ for a Fc domain of at least about 1×10⁵ liters/mole-second as well as a mutein having a k_{d} for a Fc domain of less than or equal to 3×10⁻⁵/second. More preferred is a mutein having a kₐ for a Fc domain of at least about 3×10⁵ liters/mole-second, and even more preferably of 1×10⁶ liters/mole-second. Also preferred are muteins having a k_{d} for a Fc domain of less than or equal to 1×10⁻⁵/second or even more preferably less than or equal to 3×10⁻⁴/second. A preferred Fc domain is that of an IgE antibody. Methods to measure such binding constants is well known to those skilled in the art; see, for example, Cook et al., 1997, ibid., which reports the following values for the binding of human FcεRIα protein to human IgE: kₐ₁ of 3.5 (±0.9)×10⁵ M⁻¹s⁻¹; kₐ₂ of 8.6 (±3.5)×10⁴M⁻¹s⁻¹; k_{d1} of 1.2 (±0.1)×10⁻²s⁻¹; k_{d2} of 3.2 (±0.8)×10⁻⁵s⁻¹; K_{A1} of 2.0×10⁷ M⁻¹; K_{A2} of 2.9×10⁹ M⁻¹.

Another embodiment of the present invention is a mutein that exhibits altered substrate specificity compared to its unmodified counterpart. A mutein exhibiting altered substrate specificity is a mutein that binds with increased affinity to a Fc domain of an antibody class or antibody species of a different type than that normally bound by its unmodified counterpart. In one embodiment, a mutein of a human FcεRIα protein with altered substrate specificity is a FcR that binds with increased affinity to a IgE antibody of another mammal, such as, but not limited to, a canine, feline, equine, murine, or rat IgE antibody. In another embodiment, a mutein of a human FcεRIα protein with altered substrate specificity is a FcR that binds with increased affinity to an antibody of another class, such as IgG, IgM, IgA, or IgD, with IgG being preferred. Such a mutein can also show altered species substrate specificity. Methods to determine whether a mutein exhibits altered substrate specificity are well known to those skilled in the art.

Yet another embodiment of the present invention is a mutein that exhibits increased solubility compared to its unmodified counterpart. Such a protein is less likely to form aggregates. Methods to determine whether a mutein exhibits increased solubility are well known to those skilled in the art.

As disclosed herein, the 3-D model representing a FcεRIα:Fc-Cε3/Cε4 complex is advantageous in determining strategies for producing muteins having an improved function, e.g., for identifying targets to modify in order to obtain muteins having improved functions. Examples of targets are as follows. A key feature of the human FcεRIα₁₋₁₇₆ protein is the crystal contacts in five space groups, a subset of which are predicted to interact directly with a Fc domain of an IgE antibody. Such contacts are included in the IgE binding domain which is unique for human FcεRIα in that the domain includes a tryptophan-containing hydrophobic ridge positioned on the top face of the crystal structure (i.e., amino acids W87, W10, W113, and W156 of SEQ ID NO:2) and an FG loop comprising amino acids from 155 to 158 of SEQ ID NO:2 that protrudes above the interface in an unusual manner. Particularly preferred amino acids are residues at positions of 85, 86, 87, and 110 of SEQ ID NO:2. Another key feature is the interface between domain 1 and domain 2 (i.e., the D1D2 interface) which includes amino acids 12, 13, 14, 15, 16, 17, 18, 20, 84, 85 and 86 in D1 and 87, 88, 89, 90, 91, 92, 93, 95, 104, 106, 108, 110, 111, 161, 163, 164, and 165 in D2 of SEQ ID NO:2. Also important are the two domains themselves: D1 includes amino acids 1 through 86 of SEQ ID NO:2; and D2 includes amino acids 87 through 176 of SEQ ID NO:2. Another important feature is the cleft between D1 and D2, which can be identified using the coordinates. Other areas of interest include the hydrophobic core which can be identified using the coordinates, the A′B loop of D1, which includes amino acids 18 and 19, the EF loop of D1, which includes amino acids 59-63, the BC loop of D2, which includes amino acids 110-114, the C strand of D2, which includes amino acids 114-123, the CC′ loop of D2, which includes amino acids 123-125, the C′E loop of D2, which includes amino acids 127-134, in the different confirmations observed in the five crystal forms, and the F strand of D2, which includes amino acids 147-155 of SEQ ID NO:2. Yet another striking feature is the finding that the amino and carboxyl termini of the human FcεRIα₁₋₁₇₆ protein are only 10 angstroms apart. Particularly preferred targets are a crystal contact cluster, a tryptophan-containing hydrophobic ridge, a FG loop in D2, a D1D2 interface, a cleft between D1 and D2, a domain 1, a domain 2, a hydrophobic core, a A′B loop of D1, a EF loop of D1, a BC loop of D2, a C strand of D2, a CC′ loop of D2, a C′E loop of D2, a strand of D2, the amino terminal five residues of said protein, and the carboxyl terminal five residues of said protein, with FcεRIα:Fc-Cε3/Cε4 interaction site 1, a FcεRIα:Fc-Cε3/Cε4 interaction site 2, a C strand of domain 2 of Fc8RIα, a C′E loop of domain 2 of FcεRIα, and a tryptophan-containing hydrophobic ridge of FcεRIα being particularly preferred. Preferred residues to target include residues at positions 85, 86, 87, 110, 113, 117, 119, 126,129,130, 131, 132,156, 157 and 158 of SEQ ID NO:2. In one embodiment, preferred regions to target are listed in Tables 3, 4, and 5.

TABLE 3
Contact analysis between specified sets of atoms in FcεRIα:Fc-Cε3/Cε4
interaction site 1

set1 = ( segid A )
set2 = ( segid B )
definition of contact atoms: ( known and not hydrogen )
maximum distance cutoff between contact atoms: 4.0
List of contacting residue pairs between set1 and set2. The atoms
that form the closest contact between the particular pair of residues
and the corresponding distance are listed.

| atom in set 1 | atom in set 2 | distance (A) |
|---|---|---|
| [ LYS 117 NZ ] | [ GLY 335 O ] | 3.24203 |
| [ LYS 117 NZ ] | [ ASP 362 OD2 ] | 3.40928 |
| [ ILE 119 CD1 ] | [ ASN 394 O ] | 2.99234 |
| [ ALA 126 CB ] | [ ARG 393 O ] | 3.47281 |
| [ ALA 126 CB ] | [ ASN 394 C ] | 3.8627 |
| [ ALA 126 CB ] | [ GLY 395 N ] | 3.50267 |
| [ TYR 129 OH ] | [ ASP 362 O ] | 2.80047 |
| [ TYR 129 CE2 ] | [ ALA 364 CB ] | 3.81077 |
| [ TRP 130 CZ2 ] | [ ARG 334 NH2 ] | 3.40032 |
| [ TRP 130 CZ3 ] | [ HIS 424 CE1 ] | 3.908 |
| [ TYR 131 CG ] | [ ARG 334 CG ] | 3.15693 |
| [ TYR 131 CE2 ] | [ VAL 336 CG2 ] | 3.33025 |
| [ TYR 131 CE2 ] | [ ASP 362 O ] | 3.72658 |
| [ TYR 131 OH ] | [ ALA 364 N ] | 3.33849 |
| [ TYR 131 OH ] | [ HIS 424 ND1 ] | 2.60229 |
| [ GLU 132 OE1 ] | [ ARG 334 NH1 ] | 2.4186 |

TABLE 4
Contact analysis between specified sets of atoms in FcεRIα:Fc-Cε3/Cε4
interaction site 1

set1 = ( segid A )
set2 = ( segid D )
definition of contact atoms: ( known and not hydrogen )
maximum distance cutoff between contact atoms: 4.0
List of contacting residue pairs between set1 and set2. The atoms
that form the closest contact between the particular pair of residues
and the corresponding distance are listed.

| atom in set 1 | atom in set 2 | distance (A) |
|---|---|---|
| [ SER 85 OG ] | [ PRO 426 O ] | 3.61996 |
| [ SER 85 O ] | [ ARG 427 CG ] | 3.88945 |
| [ ASP 86 O ] | [ PRO 426 CB ] | 3.23037 |
| [ ASP 86 OD2 ] | [ ARG 427 CD ] | 3.37831 |
| [ TRP 87 CH2 ] | [ LEU 425 CD2 ] | 3.4993 |
| [ TRP 87 CZ2 ] | [ PRO 426 CD ] | 3.58257 |
| [ TRP 87 NE1 ] | [ ARG 427 N ] | 3.96531 |
| [ TRP 110 CG ] | [ PRO 426 CG ] | 3.30731 |
| [ TRP 113 CH2 ] | [ HIS 424 O ] | 3.3407 |
| [ TRP 156 CA ] | [ PRO 333 O ] | 3.71511 |
| [ TRP 156 O ] | [ ARG 334 CA ] | 3.63918 |
| [ TRP 156 O ] | [ GLY 335 N ] | 3.19027 |
| [ GLN 157 NE2 ] | [ CYS 329 N ] | 3.96932 |
| [ GLN 157 NE2 ] | [ ASN 332 ND2 ] | 2.70954 |
| [ GLN 157 NE2 ] | [ PRO 333 O ] | 3.96239 |
| [ GLN 157 OE1 ] | [ ARG 334 NH1 ] | 3.22424 |
| [ LEU 158 CD1 ] | [ GLY 335 O ] | 3.71969 |
| [ LEU 158 CD1 ] | [ VAL 336 O ] | 3.42542 |
| [ MAN 246 O2 ] | [ ARG 427 NH2 ] | 3.54884 |

TABLE 3
Contact analysis between specified sets of atoms in FcεRIα-CHAPs
interaction

set1 = ( segid A )
set2 = ( segid E )
definition of contact atoms: ( known and not hydrogen )
maximum distance cutoff between contact atoms: 4.0
List of contacting residue pairs between set1 and set2. The atoms
that form the closest contact between the particular pair of residues
and the corresponding distance are listed.

| atom in set 1 | atom in set 2 | distance (A) |
|---|---|---|
| [ ARG 111 NH1 ] | [ CPS 101 O4 ] | 3.46342 |
| [ TRP 113 NE1 ] | [ CPS 101 O4 ] | 3.2081 |
| [ TRP 113 CZ2 ] | [ CPS 103 C16 ] | 3.9932 |
| [ TYR 116 CB ] | [ CHA 102 O8 ] | 3.23437 |
| [ LYS 117 CD ] | [ CHA 102 O6 ] | 3.86424 |
| [ LYS 154 CD ] | [ CHA 102 O6 ] | 3.11731 |
| [ TRP 156 CZ2 ] | [ CPS 103 C11 ] | 3.36681 |
| [ GLN 157 CG ] | [ CHA 102 O7 ] | 3.90519 |

In accordance with the present invention, a mutein having an improved function can be produced by a method that includes replacing at least one amino acid based on information derived from analyzing a 3-D model of the present invention to produce the mutein having the improved function. Knowledge of the structure of the extracellular domain of a human FcεRIα protein crystal, for example, permits the rational design and construction of modified forms of the protein by permitting the prediction and production of substitutions, insertions, deletions, inversions and/or derivatizations that effect an improved function. That is, analysis of 3-D models of the present invention provide information as to which amino acid residues are important and, as such, which amino acids can be changed without harming the protein. In making amino acid replacements, it is preferred to use amino acid replacements that have similar numbers of atoms and that allow conservation of salt bridges, hydrophobic interactions and hydrogen bonds unless the goal is to purposefully change such interactions. The 3-D structure of the human FcεRIα protein suggests that large deletions may not be desirable, particularly due to the relation between the various domains of the protein and the observation that most of the structure is well ordered in the crystal. An exception to this is the non-constrained loops of D1, which apparently could be deleted or shortened without harming the protein's function. These loops span amino acids 31-35 and 70-74 of SEQ ID NO:2.

It is to be appreciated that although one amino acid replacement capable of improving the function of a protein can substantially improve that function, more than one amino acid replacement can result in cumulative changes depending on the number and location of the replacements. For example, although one amino acid replacement capable of substantially increasing the stability of a protein can increase the melting temperature of that modified protein by about 1° C., about 5 to about 6 replacements may increase the melting temperature of the resultant protein by about 10° C.

In accordance with the present invention, the 3-D model of the complex has been analyzed, using techniques known to those skilled in the art, to determine the accessibility of the amino acids represented within the model to solvent. Such information is provided in, for example, Table 2.

A number of methods can be used to produce muteins of the present invention. One method includes the steps of: (a) analyzing a 3-D model substantially representing the coordinates specified in Table 1 to identify at least one amino acid of the modeled protein which if replaced by a specified amino acid would effect an improved function; and (b) replacing the identified amino acid(s) to produce a mutein having that improved function. In one embodiment, a method to produce a mutein includes the steps of (a) comparing a key region of a model of a human FcεRIα protein with the amino acid sequence of a FcR having an improved function compared to the unmodified FcεRIα protein in order to identify at least one amino acid segment of the FcR with the improved function that if incorporated into the FcεRIα protein represented by the model would give the FcεRIα protein the improved function; and (b) incorporating the segment into the FcεRIα protein, thereby providing a mutein with the improved function. In another embodiment, a method to produce a protein includes the steps of: (a) using a model representing a human FcεRIα protein to identify a 3-D arrangement of residues that can be randomized by mutagenesis to allow the construction of a library of molecules from which a improved function can be selected; and (b) identifying at least one member of the mutagenized library having the improved function. In one example, a mutein is produced by a method that includes the steps of: (a) effecting random mutagenesis of nucleic acid molecules encoding a target of a FcεRIα protein as identified by analyzing a model of that protein, such as an IgE binding domain; (b) cloning such mutagenized nucleic acid molecules into a phage display library, wherein said phage display library expresses the target; and (c) identifying at least one member of the library that expresses a target with an improved function, such as an antibody binding domain exhibiting increased affinity for an antibody. As stated above, the model allows the use of this technique in a straightforward manner that could not be accomplished in the absence of the model. It is to be also noted that these methods can also be used with other models of the present invention to produce muteins of the present invention.

The present invention includes a number of methods, based on analysis of a 3-D model of the present invention, to replace (i.e., add, delete, substitute, invert, derivatize) at least one amino acid residue in the protein represented by the model in order to produce a mutein of the present invention. Such methods include, but are not limited to: (a) replacing at least one amino acid in at least one non-constrained loop of domain 1 in an area proximal to the FceRI gamma chain putative binding site; (b) joining an amino-terminal amino acid residue to a carboxyl-terminal amino acid residue of an extracellular domain of a FcεRIα protein; (c) replacing at least one amino acid site with an amino acid suitable for derivatization; (d) replacing at least one pair of amino acids of the protein with a cysteine pair to enable the formation of a disulfide bond that stabilizes the protein; (e) removing at least a portion of the region between the B strand and C strand of domain 1; (f) removing at least a portion of the region between the C strand and E strand of domain 1; (g) replacing at least one amino acid in the IgE binding domain in order to increase the affinity between an IgE antibody and the protein; (h) replacing at least one amino acid of the protein with an amino acid such that the replacement decreases the entropy of unfolding of the protein; (i) replacing at least one asparagine or glutamine of the protein with an amino acid that is less susceptible to deamidation than is the amino acid to be replaced; (j) replacing at least one methionine, histidine or tryptophan with an amino acid that is less susceptible to an oxidation or reduction reaction than is the amino acid to be replaced; (k) replacing at least one arginine of the protein with an amino acid that is less susceptible to dicarbonyl compound modification than is the amino acid to be replaced; (l) replacing at least one amino acid of the protein susceptible to reaction with a reducing sugar sufficient to reduce protein function with an amino acid less susceptible to that reaction; (m) replacing at least one amino acid of the protein with an amino acid capable of increasing the stability of the inner core of the protein; (n) replacing at least one amino acid of the protein with at least one N-linked glycosylation site; (o) replacing at least one N-linked glycosylation site of the protein with at least one amino acid that does not comprise an N-linked glycosylation site; and (p) replacing at least one amino acid of the protein with an amino acid that reduces aggregation of the protein.

Amino acid replacements can be carried out using recombinant DNA techniques known to those skilled in the art, including site-directed mutagenesis (e.g., oligonucleotide mutagenesis, random mutagenesis, polymerase chain reaction (PCR)-aided mutagenesis, gapped-circle site-directed mutagenesis) or chemical synthetic methods of a nucleic acid molecule encoding the desired protein, such as, but not limited to a human FcεRIα protein, followed by expression of the mutated gene in a suitable expression system, preferably an insect, mammalian, bacterial, yeast, insect, or mammalian expression system. See, for example, Sambrook et al., ibid.

One embodiment of the present invention is a mutein in which at least one amino acid in at least one non-constrained loop of a FcεRIα protein is replaced in order to improve a function of the protein. Finding that the human FcεRIα protein had such loops was surprising, and it is believed, without being bound by theory, that a mutein in which at least a portion of at least one such loop is replaced, would at least exhibit enhanced stability. In a preferred embodiment, at least a portion of one or more of such loops is (are) deleted. Preferred loops to replace are in domain 1 (i.e., spanning amino acids 31-35 and 70-74 of SEQ ID NO:2), preferably in an area proximal to the FceRI gamma chain putative binding site, i.e., the site on the FcεRIα protein to which the gamma chain of the high affinity Fc epsilon receptor is thought to bind. In a preferred embodiment, one or more amino acids is replaced to make loops shorter, but including 1 or 2 hydrophobic residues to pack toward the protein interior and at least one hydrophilic residue to maintain solubility.

Another embodiment of the present invention is a mutein of the extracellular domain of a FcεRIα protein in which an N-terminal (amino-terminal) amino acid residue is joined, preferably covalently, to a C-terminal (carboxyl-terminal) amino acid residue in order to improve a function of the protein. Finding that the N-termini and C-termini of the human FcεRIα protein were only 10 angstroms apart was quite surprising. Without being bound by theory, it is believed that such a mutein would at least exhibit enhanced stability. Furthermore, a covalent linker used to join the termini could also include a substance useful, for example, to anchor a mutein on a surface, as would be useful, for example, in a diagnostic assay, or to label the mutein. For a protein consisting of SEQ ID NO:2, a preferred N-terminal residue is an amino acid residue at position 1, 2, or 3 of SEQ ID NO:2, and a preferred C-terminal residue is an amino acid residue at position 174, 175, or 176 of SEQ ID NO:2. Covalent linkage can be accomplished by methods known to those skilled in the art, such as, but not limited to, adding one or more N-terminal and C-terminal cysteines and crosslinking them with chemical compounds, adding additional residues in the coding sequence to allow the formation of a disulfide bond, or adding one or more lysines and coupling them through a 10 angstrom linker, and including non-natural amino acid analogues by synthetic methods or by a combination of biosynthetic and organosynthetic methods. Examples of a substance to add to a covalent linker includes: ligands useful in allowing for the attachment of a mutein to a surface, such as biotin and related compounds, avidin and related compounds, metal binding compounds, sugar binding compounds, immunoglobulin binding domains, and other tag domains; and detectable markers, such as enzyme labels, physical labels, radioactive labels, fluorescent labels, chemiluminescent labels, and chromophoric labels. Examples include, but are not limited to, alkaline phosphatase, horseradish peroxidase, digoxygenin, luciferase, other light-generating enzymes and magnetic beads. It is also to be noted that ligands can function as detectable markers.

Another embodiment of the present invention is a mutein in which at least one amino acid is replaced with an amino acid suitable for derivatization. Muteins in which at least one amino acid is replaced with an amino acid suitable for derivatization include proteins that are chemically modified (e.g., a lysine already existing on the protein is modified) as well as those in which an amino acid residue is replaced with a different amino acid residue (e.g., a glycine with a lysine) as well as proteins to which a substance is added, preferably to the amino or carboxyl terminus of the protein. Examples of such substances include ligands and detectable markers as disclosed above. Preferable amino acids to replace include residues that are solvent exposed (e.g., those listed in Table 2), but that are preferably not within about 10 angstroms of the IgE binding domain. In one embodiment, a glycosylation site, or other solvent exposed site, is replaced with a charged or polar residue to increase solubility or create more stable muteins. Glycosylation sites in human FcεRIα. protein include amino acids 21, 42, 50 74, 135, 140, and 166 of SEQ ID NO:2. A preferred amino acid to use as a replacement, or to chemically modify directly, includes a cysteine or a lysine, with a cysteine being preferred. Compounds to use in chemical derivatizations are known to those skilled in the art; cysteines can, for example, be derivatized with maleimides.

Another embodiment of the present invention is a mutein in which a pair of amino acids have been replaced with a cysteine pair in order to improve the function of the mutein, at least by increasing stability. Cysteine pairs can be substituted into a FcεRIα protein at any two residue positions identified with available programs and algorithms that would allow the formation of an undistorted disulfide bridge. In one embodiment, a serine and lysine near the termini of the protein is each replaced with a cysteine. In another embodiment, cysteine pairs are replaced with other amino acids, such as serines to eliminate non-essential disulfide bonds.

Another embodiment of the present invention is a mutein in which at least one amino acid is replaced in the region between the B strand and C strand of domain 1 and/or the region between the C and E strand of domain 1. In a preferred embodiment, at least a portion of such a region is deleted.

Another embodiment of the present invention is a mutein in which at least one amino acid is replaced in the IgE binding domain in order to increase the affinity between an IgE antibody and the protein. Preferred residues to replace are in or near the IgE binding domain, or IgE binding site, as determined by analysis of the 3-D model. Such residues are preferably within about 10 angstroms of residues identified by mutagenesis and further shown by model to be in an IgE binding site. Examples of such residues include amino acids 87, 110, 113, 115, 117, 118, 120, 121, 122, 123, 128, 129, 131, 149, 153, 154, 155, 156, 157, 158, and 159 of SEQ ID NO:2, and amino acids within 10 angstroms of such listed amino acids. In one embodiment, preferred amino acids to replace include amino acids 87, 115, 117, 118, 120-123, 128, 129, 131, 149, 153, 155 and 159 of SEQ ID NO:2 as well as any surface residue within about 10 angstroms of any of the listed amino acids, with amino acids 87, 117, 121, 123, 128, 159 of SEQ ID NO:2 or SEQ ID NO:4 as well as any surface residue within about 10 angstroms of amino acids 87, 117, 121, 123, 128, 159 of SEQ ID NO:2 being particularly preferred. It is to be noted that amino acids 115, 118, 120, 131, 149 and 155 of SEQ ID NO:2 are buried, and that amino acids that are partially buried or glycine include residues 122, 129 and 153. Additional amino acid residues to target include those in the A′B loop of D1, and EF loop of D1. Note that these residues are not the same as those shown in mutation studies to affect IgE binding since some of those mutants have mutations in amino acids that are internal to the protein; this finding can only be made by analysis of a model of the present invention.

Another embodiment of the present invention is a mutein in which at least one amino acid is replaced with an amino acid capable of increasing the stability of the inner core or surface of the protein. Preferred amino acids to replace are hydrophilic residues located in the hydrophobic core of the protein and/or hydrophobic amino acids at the protein surface that are not within about 10 angstroms of the IgE binding domain residues of D1 or D2. Preferred amino acids to replace into the hydrophobic core are hydrophobic residues such as, but not limited to, tryptophan, leucine, isoleucine, valine and alanine, as well as space filling amino acids, such as other aromatic amino acids. Preferred amino acids to replace onto the surface are polar amino acids, such as, but not limited to, glutamic acid, glutamine, aspartic acid, asparagine, histidine and serine. Muteins having one or more such amino acid replacements would exhibit at least increased stability and/or reduced aggregation. Additional preferred amino acid replacements are those that introduce salt bridges at the protein surface to stabilize protein folds. It is noted that the cysteines at positions 26 and 68 of SEQ ID NO:2 form a disulfide bond in domain 1 that is somewhat exposed to solvent, depending especially on the conformation of the Dl “30 loop” (i.e., amino acids 31-35 of SEQ ID NO:2). In one embodiment, changes in neighboring residues can be made in, for example, residues 1-5, 27-37, 49-52, or 69-75, to bury this disulfide from exposure to solvent. For example, phage display of receptors with randomized mutations in the 30 loop, might be useful for selecting receptors that react less well with reducing reagents and have a more stable D1 core.

Another embodiment of the present invention is a mutein in which at least one amino acid is replaced with an amino acid that decreases the entropy of unfolding of the protein. The entropy of unfolding of a protein can be measured and compared to that of another protein using techniques known to those skilled in the art. A number of methods known to those skilled in the art can be used to reduce the number of protein conformations possible in the unfolded state, thereby improving the ability of the protein to fold correctly. One embodiment of the present invention for decreasing the entropy of unfolding includes replacing at least one amino acid of the protein with a specified amino acid in order to maintain certain desirable phi and psi backbone conformation angles in the protein; see, for example, PCT International Publication No. WO 89/01520, by Drummond et al., published Feb. 23, 1989. For example, a proline residue in a protein constrains the backbone conformation to certain restricted angles. Analysis of a 3-D model of a protein of the present invention permits the identification of candidate replacement positions in the protein that have the conformation expected for a proline, but that do not have a proline in them. Such knowledge is used to introduce prolines into such candidate replacement positions to “anchor” the resultant mutein in the desired conformation. The 3-D model also permits the identification of candidate replacement sites that if replaced with a proline do not substantially disrupt the 3-D structure of the resultant protein. Similarly, glycines in appropriate positions can be replaced with an amino acid having a β carbon atom or a branched carbon atom, preferably an alanine, in order to stabilize the backbone of the protein.

Another embodiment of the present invention is a mutein in which at least one asparagine or glutamine is replaced with an amino acid that is less susceptible to deamidation. Preferred amino acids to replace include solvent accessible asparagines and glutamines.

Another embodiment of the present invention is a mutein in which at least one methionine, histidine or tryptophan is replaced with an amino acid that is less susceptible to an oxidation or reduction reaction. Preferred amino acids to replace include M98, H70, and H41. It would not be preferred to replace any of the tryptophans, nor H108 or H134 of SEQ ID NO:2.

Another embodiment of the present invention is a mutein in which at least one arginine is replaced with an amino acid that is less susceptible to dicarbonyl compound modification. Although R174 could be changed, it would probably not be preferable to change amino acids at the D1D2 interface or near the IgE binding site, such as amino acids 15, 106, or 111 of SEQ ID NO:2.

Another embodiment of the present invention is a mutein in which at least one amino acid that is susceptible to reaction with a reducing sugar sufficient to reduce protein function is replaced with an amino acid that is less susceptible to such a reaction. For example, lysines, glutamines and asparagines that could react with a sugar, such as galactose, glucose or lactose can be replaced with non-reactive amino acids.

Another embodiment of the present invention is a mutein in which one or more N-linked glycosylation sites are added to or removed from the protein, preferably by substitution with an appropriate amino acid. A FcεRIα protein with additional N-linked glycosylation sites is more soluble. The ability to design a FcεRIα protein having fewer, or no, N-linked glycosylation sites is also valuable as production of such a protein from production run to production run is likely to be more uniform. One embodiment is a FcεRIα mutein with no N-linked glycosylation sites that is stable, active, and soluble. Such a protein has an advantage of being produced in E. coli at low cost. In one embodiment, one or more exposed hydrophobic amino acids are changed to charged residues that form salt bridges to stabilize the protein fold and make it soluble. It is to be noted that the glycosylation sites that appear to be most often observed in the different crystal structures in the same conformation are the carbohydrate attached to positions 42 and 166 of SEQ ID NO:2. The carbohydrate attached to position 42 always appears to cover the phenylalanine at position 60 of SEQ ID NO:2. As such, one embodiment of the present invention is to remove the glycosylation site at position 42, e.g., by substitution with a suitable amino acid. This embodiment has the additional advantage that the resultant mutein has an exposed phenylalanine at position 60, thereby leading to increased IgE binding activity.

Another embodiment of the present invention is a mutein in which at least one amino acid is replaced with an amino acid that reduces aggregation and increases solubility of the protein, such as, for example, replacing one or more hydrophobic residues on the surface with one or more hydrophilic residues. Other examples of such amino acids to replace are disclosed herein.

Another embodiment of the present invention to enhance stability is the addition of polyethylene glycol (PEG) groups to a FcR protein, i.e., to produce a “pegylated” FcR protein. In one embodiment, the PEG group(s) can substitute for carbohydrate group(s) due to removal of one or more N-glycosylation sites. Such PEG group(s) can be attached to easily modifiable residues, such as cysteines or lysines, on the surface of the protein, such residues identifiable by analysis of a 3-D model of the present invention.

Another embodiment of the present invention is a mutein that comprises a FcR having a substance, such as a ligand or detectable marker, attached to an amino acid of the protein such that the substance does not substantially interfere with the antibody binding activity of the protein. The substance is attached in such a manner that the substance is also capable of performing its function, such as binding to a second member of a ligand pair or enabling detection of the protein. The FcR to which a substance is attached can be either an unmodified protein or a mutein of the present invention. Suitable attachment sites can be identified using 3-D models of the present invention. Preferred attachment sites include solvent exposed amino acids, such as those listed in Table 2. Substances can be attached, or conjugated, to the protein using techniques known to those skilled in the art. It is to be appreciated that a preferred method to attach a substance to an amino acid is to modify that amino acid to have a reactive attachment site, such as is present on cysteine and lysine amino acids. As such, an attachment site comprising a solvent exposed amino acid refers to the nature of the amino acid prior to any modification required for attachment. Examples of suitable substances to attach to a FcR include any compound capable of binding to or reacting with another substance, such as those described for attachment to a covalent linker.

It is to be appreciated that muteins of the present invention can include amino acids which are not modified because they would negatively impact the function of the protein. Such amino acids can be identified using a 3-D model of the present invention.

It should also be appreciated that it is within the scope of the present invention to expand the use of models of the present invention to produce models of and make modifications to any suitable FcRs or other Ig domain-containing proteins to produce muteins having a desired function.

The present invention also includes a mutein that binds to an IgE binding domain of a FcεRIα protein, wherein the mutein has an improved function compared to a Fc-Cε3/Cε4 protein comprising amino acid sequence SEQ ID NO:6. Such an improved function can include increased stability compared to the stability of a human IgE Fc region comprising amino acid sequence SEQ ID NO:6, increased affinity for a FcεRIα protein compared to the FcεRIα affinity of a human IgE Fc region comprising amino acid sequence SEQ ID NO:6, altered substrate affinity compared to the affinity for human FcεRIα of a human IgE Fc region comprising amino acid sequence SEQ ID NO:6, and increased solubility compared to the solubility of a human IgE Fc region comprising amino acid sequence SEQ ID NO:6. Such a mutein is produced by a method that includes the steps of (a) analyzing a three-dimensional model substantially representing the atomic coordinates specified in Table 1 to identify at least one amino acid of the Fc-Cε3/Cε4 protein represented by said model which if replaced by a specified amino acid would effect said improved function of said Fc-Cε3/Cε4 protein; and (b) replacing said identified amino acid(s) to produce said mutein having said improved function. Fc muteins can be identified and produced in a manner similar to that described herein for FcR muteins. Antibody muteins have a variety of uses, including but not limited to, diagnostic and therapeutic uses. For example, muteins could be used to image cells that express an antibody receptor protein, such as NMR-specific labeling for in vivo imaging to detect, for example, mast cell cancers, asthma, and other pathologies, or to treat cancers that express an antibody receptor protein using, for example, radioimmune therapy of derivatized IgE. Muteins could also be used for monitoring FcR expression in atopic individuals (e.g. with a tag for one-step FACS analysis) or for monitoring IgE in atopic individuals. Muteins could also be used as inhibitors or as toxin-IgE-Fc fusion proteins to target FcR-expressing cells to kill them (e.g. in mast cell tumors or severe allergy). Also muteins that affect the low affinity affinity IgE-receptor (FceRII) binding but not FceRI binding could be designed or selected.

The present invention also includes nucleic acid molecules that encode muteins of the present invention as well as recombinant molecules and recombinant cells that include such nucleic acid molecules. Methods to produce such proteins are also disclosed herein.

The present invention also includes the following novel structures as identified by a 3-D model of the present invention. Preferred structures exhibiting direct interaction between IgE and FcεRIα include FcεRIα:Fc-Cε3/Cε4 interaction site 1, a FcεRIα:Fc-Cε3/Cε4 interaction site 2, a C strand of domain 2 of FcεRIα, a C′E loop of domain 2 of FcεRIα, and a tryptophan-containing hydrophobic ridge of FcεRIα. Other preferred structures include a crystal contact cluster involved in IgE binding; a FG loop in D2; a D1D2 interface; a cleft between D1 and D2; a domain 1; a domain 2; a hydrophobic core; a A′B loop of D1; a EF loop of D1; a BC loop of D2; a CC′ loop of D2; and a strand of D2. Particularly preferred are (a) a FcεRIα:Fc-Cε3/Cε4 interaction site 1 pocket comprising an amino acid residue at position 131 of SEQ ID NO:2 and amino acid residues at positions 9, 11, 37, 39, and 99 of SEQ ID NO:6 and (b) a FcεRIα:Fc-Cε3/Cε4 interaction site 2 pocket comprising amino acid residues at positions 85, 86, 87, and 110 of SEQ ID NO:2 and amino acid residue at position 101 of SEQ ID NO:6. Also included herein are nucleic acid molecules to encode such structures as well as recombinant molecules and recombinant cells that include such nucleic acid molecules. Also included are methods to produce such structures and models thereof.

The present invention also includes isolated nucleic acid molecules encoding proteins of the present invention, including, but not limited to, unmodified proteins, novel structures within such proteins, and muteins. As used herein, an isolated nucleic acid molecule encoding a protein is a nucleic acid molecule that has been removed from its natural milieu. As such, “isolated” does not reflect the extent to which the nucleic acid molecule has been purified. An isolated nucleic acid molecule can be DNA, RNA, or derivatives of either DNA or RNA.

A nucleic acid molecule encoding a mutein of the present invention can be produced by mutation of parental protein genes (e.g., unmodified or previously modified protein-encoding genes, or portions thereof) using recombinant DNA techniques heretofore disclosed or by chemical synthesis. Resultant mutein nucleic acid molecules can be amplified using recombinant DNA techniques known to those skilled in the art, such as PCR amplification or cloning (see, for example, Sambrook et al., ibid.), or by chemical synthesis. A mutein can also be produced by chemical modification of a protein expressed by a nucleic acid molecule encoding an unmodified protein or mutein-encoding gene.

Proteins of the present invention can be produced in a variety of ways, including production and recovery of recombinant proteins and chemical synthesis. In one embodiment, a protein of the present invention is produced by culturing a cell capable of expressing the protein under conditions effective to produce the protein, and recovering the protein. A preferred cell to culture is a recombinant cell that is capable of expressing the protein, the recombinant cell being produced by transforming a host cell with one or more nucleic acid molecules of the present invention. Transformation of a nucleic acid molecule into a host cell can be accomplished by any method by which a nucleic acid molecule can be inserted into a cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules of the present invention can remain extrachromosomal or can integrate into one or more sites within a chromosome of a host cell in such a manner that their ability to be expressed is retained.

Suitable host cells to transform include any cell that can be transformed. Host cells can be either untransformed cells or cells that are already transformed with at least one nucleic acid molecule. Host cells of the present invention can be endogenously (i.e., naturally) capable of producing a protein of the present invention, but such cells are not preferred. Host cells of the present invention can be any cell that when transformed with a nucleic acid molecule of the present invention are capable of producing a protein of the present invention, including bacterial, yeast, other fungal, insect, animal, and plant cells. Preferred host cells include bacterial, yeast, insect and mammalian cells, and more preferred host cells include Escherichia, Bacillus, Saccharomyces, Pichia, Trichoplusia, Spodoptera and mammalian cells. Particularly preferred host cells are Trichoplusia ni cells and Spodoptera frugiperda cells with T. ni cells being particularly preferred.

A recombinant cell is preferably produced by transforming a host cell with a recombinant molecule comprising a nucleic acid molecule of the present invention operatively linked to an expression vector containing one or more transcription control sequences. The phrase operatively linked refers to insertion of a nucleic acid molecule into an expression vector in a manner such that the molecule is able to be expressed when transformed into a host cell. As used herein, an expression vector is a DNA or RNA vector that is capable of transforming a host cell, of replicating within the host cell, and of effecting expression of a specified nucleic acid molecule. Expression vectors can be either prokaryotic or eukaryotic, and are typically viruses or plasmids. Expression vectors of the present invention include any vectors that function (i.e., direct gene expression) in recombinant cells of the present invention, including in bacterial, yeast, other fungal, insect, animal, and plant cells. Preferred expression vectors of the present invention can direct gene expression in bacterial, yeast, insect and mammalian cells.

Nucleic acid molecules of the present invention can be operatively linked to expression vectors containing regulatory control sequences such as promoters, operators, repressors, enhancers, termination sequences, origins of replication, and other regulatory control sequences that are compatible with the host cell and that control the expression of the nucleic acid molecules. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, insect and mammalian cells.

It may be appreciated by one skilled in the art that use of recombinant DNA technologies can improve expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgamo sequences), modification of nucleic acid molecules of the present invention to correspond to the codon usage of the host cell, deletion of sequences that destabilize transcripts, and use of control signals that temporally separate recombinant cell growth from recombinant protein production during fermentation. The activity of an expressed recombinant protein of the present invention may be improved by fragmenting, modifying, or derivatizing nucleic acid molecules encoding such a protein.

In accordance with the present invention, recombinant cells can be used to produce proteins by culturing such cells under conditions effective to produce such a protein, and recovering the protein. Effective conditions to produce a protein include, but are not limited to, appropriate media, bioreactor, temperature, pH and oxygen conditions that permit protein production. An appropriate medium refers to any medium in which a cell of the present invention, when cultured, is capable of producing the protein. An effective medium is typically an aqueous medium comprising assimilable carbohydrate, nitrogen and phosphate sources, as well as appropriate salts, minerals, metals and other nutrients, such as vitamins. The medium may comprise complex nutrients or may be a defined minimal medium. Cells of the present invention can be cultured in conventional fermentation bioreactors, which include, but are not limited to, batch, fed-batch, cell recycle, and continuous fermentors. Culturing can also be conducted in shake flasks, test tubes, microtiter dishes, and petri plates. Culturing is carried out at a temperature, pH and oxygen content appropriate for the recombinant cell. Such culturing conditions are well within the expertise of one of ordinary skill in the art.

Depending on the vector and host system used for production, resultant proteins may either remain within the recombinant cell; be secreted into the fermentation medium; be secreted into a space between two cellular membranes, such as the periplasmic space in E. coli; or be retained on the outer surface of a cell or viral membrane. The phrase “recovering the protein” refers simply to collecting the whole fermentation medium containing the protein and need not imply additional steps of separation or purification. Proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, chromatofocusing and differential solubilization.

The present invention also includes isolated (i.e., removed from their natural milieu) antibodies that selectively bind to a FcR or antibody of the present invention. As used herein, the term “selectively binds to” refers to the ability of antibodies of the present invention to preferentially bind to specified proteins of the present invention. Binding can be measured using a variety of methods standard in the art including enzyme immunoassays (e.g., ELISA), immunoblot assays, etc.; see, for example, Sambrook et al., ibid. Isolated antibodies of the present invention can include antibodies in a bodily fluid (such as, but not limited to, serum), or antibodies that have been purified to varying degrees. Antibodies of the present invention can be polyclonal or monoclonal. Functional equivalents of such antibodies, such as antibody fragments and genetically-engineered antibodies (including single chain antibodies or chimeric antibodies that can bind to more than one epitope) are also included in the present invention. Antibodies can be produced using methods known to those skilled in the art. A preferred method to produce antibodies of the present invention includes (a) administering to an animal an effective amount of a protein of the present invention to produce the antibodies and (b) recovering the antibodies. In another method, antibodies of the present invention are produced recombinantly using techniques as heretofore disclosed to produce proteins of the present invention. Antibodies raised against defined proteins can be advantageous because such antibodies are not substantially contaminated with antibodies against other substances that might otherwise cause interference in a diagnostic assay or side effects if used in a therapeutic composition.

Antibodies of the present invention have a variety of potential uses that are within the scope of the present invention. Examples of such uses are disclosed in WO 98/27208, ibid., see, for example, page 24; such uses are incorporated by reference herein in their entireties.

A FcR of the present invention can include chimeric molecules comprising at least a portion of a FcR that binds to an antibody and a second molecule that enables the chimeric molecule to be bound to a substrate in such a manner that the antibody receptor portion binds to the antibody in at least as effective a manner as a FcR that is not bound to a substrate. An example of a suitable second molecule includes a portion of an immunoglobulin molecule or another ligand that has a suitable binding partner that can be immobilized on a substrate, e.g., biotin and avidin, or a metal-binding protein and a metal (e.g., His), or a sugar-binding protein and a sugar (e.g., maltose). An antibody of the present invention can also be part of a chimeric molecule.

The present invention includes uses of proteins, antibodies and inhibitory compounds of the present invention for the diagnosis and treatment of allergy and the regulation of other immune responses in an animal.

One embodiment is a therapeutic composition comprising at least one of the following therapeutic compounds: an inhibitory compound of the present invention, a mutein of the present invention, or an antibody of the present invention. Also included is a method to protect an animal from allergy or other abnormal immune responses. Such a method includes the step of administering a therapeutic composition of the present invention to the animal. As used herein, the ability of a therapeutic composition of the present invention to protect an animal from allergy or other abnormal immune responses refers to the ability of that composition to, for example, treat, ameliorate or prevent allergy or other abnormal immune responses. General characteristics of therapeutic compositions and methods to produce and use such therapeutic compositions are disclosed, for example, in WO 98/27208, ibid., see, for example, page 39-47; such compositions and methods are incorporated by reference herein in their entireties. It is to be noted that although the compositions and methods disclosed in WO 98/27208, ibid., relate to feline FcεRIα proteins, they are also applicable to therapeutic compositions of the present invention. Therapeutic compositions of the present invention are advantageous because they can be derived from analysis of 3-D models of the present invention and have improved functions, such as efficacy and safety.

Another embodiment is a diagnostic reagent comprising a mutein of the present invention. As used herein, a diagnostic reagent is a composition that includes a mutein that is used to detect allergy or other abnormal immune responses in an animal. Also included in the present invention are methods, including in vivo methods and in vitro methods, to (a) detect allergy or other abnormal immune response, or susceptibility thereto, in an animal, comprising use of a diagnostic reagent comprising a mutein of the present invention and (b) to enhance the performance of an IgE binding assay, said method comprising incorporating into the assay a mutein of the present invention. General characteristics of diagnostic reagents and methods to produce and use such diagnostic reagents are disclosed, for example, in WO 98/27208, ibid., see, for example, page 2-39; such reagents and methods are incorporated by reference herein in their entireties. It is to be noted that although the reagents and methods disclosed in WO 98/27208, ibid., relate to feline FcεRIα proteins, they are also applicable to diagnostic reagents, kits and detection methods of the present invention. Muteins of the present invention are advantageous in such applications because of their enhanced affinity for antibodies, altered specificity, enhanced solubility and/or enhanced stability, enabling for example use in otherwise adverse conditions and longer shelf-life.

The following examples are provided for the purposes of illustration and are not intended to limit the scope of the invention.

### EXAMPLE

This Example describes the production and analysis of a crystal and model of the present invention. It is to be noted that numbering of Fc-Cε3/Cε4 residues follows the convention of Dorrington et al, ibid.

The initiation of IgE-mediated allergic responses requires the binding of IgE antibody to its high affinity receptor, FcεRI. Crosslinking of FcεRI initiates an intracellular signal transduction cascade that triggers the release of mediators of the allergic response. The interaction of IgE-Fc domains with FcεRI is a key recognition event that is central to this process and mediated by the extracellular domains of the α-chain of FcεRI. This Example describes the solution of a crystal structure of the human IgE-Fc:FcεRIα complex, the coordinates of which are disclosed in Table 1. The crystal structure reveals that one receptor binds one IgE-Fc asymmetrically through interactions at two sites involving both N-terminal IgE-Fc Cε3 domains. The interaction of one receptor with IgE-Fc blocks the high-affinity binding of a second receptor and features of this interaction are conserved in other Fc receptor family members. The structural analysis suggests new approaches to the inhibition of IgE binding to FcεRI for the treatment of allergy and asthma.

A. Introduction

The high affinity IgE receptor (FcεRI) is found on the surface of effector cells of the immune system that initiate cellular reactions associated with the allergic response, anaphylaxis and anti-parasitic immunity^{1,2}. The human receptor can form either a trimeric αγ₂ or tetrameric αβγ₂ structure on cell surfaces, with the extracellular domains of the α-chain conferring the ability to bind antibodies of the IgE class with high affinity (K_{D}˜10⁻⁹-10⁻¹⁰M). IgE antibodies bind to the receptor in the absence of antigen and thus the receptor adopts the antigenic specificity of the prevalent IgE repertoire. Crosslinking of the receptor through the engagement of antigen:antibody interactions leads to the initiation of a lyn and syk kinase-mediated signal transduction cascade, analogous to that induced by T and B cell receptors³⁻⁵. In mast cells, receptor activation leads to rapid degranulation and release of histamine followed by the synthesis and release of prostaglandins, leukotrienes, cytokines and other mediators of the allergic response. Anti-parasitic responses can be triggered through a similar activation of eosinophils, leading to the release of granular proteins toxic to schistosomes and other parasites. FcβRI belongs to a family of antibody-binding receptors that also mediate interactions of soluble IgG and IgA antibodies with cells of the immune system^{3,5}. IgG-Fc receptors regulate inflammation pathways, B cell development, and Natural Killer Cell activation and are therefore important in many aspects of immunity and disease.

Atopic diseases, such as allergy, asthma, and eczema, comprise a wide spectrum of pathologies associated with the inappropriate activation of the immune system to environmental antigens^{6,7}. Dramatic increases in atopic disease have been observed in this century, particularly in developed countries. Allergic diseases have been associated with the IgE network through genetic studies in both mice and humans, suggesting a role for polymorphisms of the FcεRI β-chain and CD14 in atopic individuals^{7,8}. The interaction of the IgE antibody with FcεRI is central to these immune reactions, providing an attractive target for the inhibition of all IgE-mediated allergic disease. Clinical studies of allergic individuals using anti-IgE monoclonal antibody therapy has demonstrated that this is a viable approach to disease treatment^{9,10}. Further development of treatments for allergy, asthma and anaphylaxis, may benefit from structural insights into the IgE:FcεRI interaction.

A recent report disclosed the crystal structure of the human FcεRI α-chain ectodomains¹¹, which revealed a highly bent arrangement of two immunoglobulin domains. Four solvent-exposed tryptophans cluster at the top of the receptor, forming a large hydrophobic surface for potential interactions with the IgE-Fc. This tryptophan cluster borders the Fc binding-site mapped by mutagenesis studies, which implicate residues in the second domain of the receptor in IgE binding. The structural and functional data suggested that a large convex surface of the receptor could be involved in binding IgE, raising questions about the role of the tryptophans, the convex nature of the binding site and the mechanisms underlying the stoichiometry and binding specificity with IgE.

These questions are addressed with the solution of a crystal structure of a complex of the human IgE-Fc with FcεRI as disclosed herein as well as of a crystal structure of the unbound IgE-Fc fragment as disclosed in 60/189,403, ibid. The structure of the complex reveals two interaction sites for the IgE-Fc on the receptor surface and clarifies how a 1:1 complex between antibody and receptor is formed. The two IgE-Fc Cε3 domains bind to distinct sites on the receptor; one is formed by the C-C′ loop in the receptor D2 domain, while the second site involves the four solvent-exposed tryptophans. The IgE Cε4 domains do not form direct contacts with the receptor and point away from the Cε3 interaction sites. The structure of the complex accounts for previous mutagenesis and structural observations and shows that the Fc forms a complementary crown across the convex surface of the receptor. Comparison of the complex with the isolated IgE-Fc crystal structure suggests that large structural changes may occur upon IgE binding to its receptor (see No. 60/189,403, ibid.) The IgE-Fc:FcεRIα complex provides a model for understanding the function of other antibody Fc-receptors and new conceptual approaches to the inhibition of IgE-mediated diseases.

B. Structure Determination of the Complex

The crystallization of the IgE-Fc:FcεRIα complex required the expression of each protein using recombinant baculovirus technology. The expression of the FcεRIα was carried out essentially as described previously¹¹. The IgE heavy chain contains four constant domains (Cε1-Cε4), in contrast to the three found in IgG antibodies. The interaction of FcεRI with IgE has been previously mapped to the two C-terminal constant domains of the IgE-Fc (domains Cε3/Cε4)¹²⁻¹⁶. The expression and purification of the human IgE-Fc Cε3/Cε4 domains was established as described (No. 60/189,403, ibid.) and purified protein used to form complexes with FcεRIα. The best complex crystals (spacegroup P42₁2) obtained with the wild type (wt) FcεRIα protein were small (˜60-100 μ/edge) and diffraction data was limited to a resolution of ˜4.5 Å (Table 3, crystal form I). In order to improve the complex crystals, a triple carbohydrate mutant of FcεRIα (FcεRIαΔ4-6) was expressed in insect cells. The FcεRIαΔ4-6 mutant lacks carbohydrate at three of the seven native attachment sites (residues 74, 134, 140) and was previously shown to produce ˜50% of the wt protein in CHO cells¹⁷. Complexes formed with baculovirus-expressed FcεRIαα4-6 grow crystals in spacegroup R32 and diffract X-rays to a resolution of 3.25 Å (Table 3, crystal form II). The structure was determined by molecular replacement techniques as described in Methods. Manual model building was done with the program O¹⁸ and refinement carried out with CNS¹⁹. Current refinement statistics for the complex are shown in Table 3, with an overall R-free of 29.3% and R-cryst of 27.0% to 3.25 Å. FIG. 1a shows electron density from a sigmaa-weighted 2Fo-Fc simulated annealing omit map calculated with the current model phases.

C. Overview of the Complex

Both crystal forms of the IgE-Fc:FcεRIα complex contain a single 1:1 complex in the asymmetric unit, with similar overall geometric features (FIGS. 1b, c). Given the low resolution of crystal form I, detailed interpretation of the interfaces is limited to crystal form II. Binding interactions are formed exclusively between the N-terminal Cε3 domains of the IgE-Fc with FcεRIα. The Cε4 domains of the IgE-Fc point away from the receptor structure and make no contacts with either receptor domain. The Cε3/Cε4 hinge regions are also not involved in direct receptor contacts. The two Cε3 domains are related by a nearly perfect diad axis (180.7° rotation), except for residues in the Cε2/Cε3-linker region (residues 331-336) (FIGS. 1b, c). The Cε4 domains are also related by a nearly perfect diad axis (179.6° rotation), but the orientation of this axis differs from that determined for the Cε3 domains (FIGS. 1b,c). The angle between the Cε3 and Cε4 domains also differs from that seen in the IgE-Fc alone (see 60/189,403, ibid.) While structured carbohydrate is visible in both the IgE-Fc and FcεRIα proteins, the carbohydrate groups do not contribute significantly to interactions between the two molecules. In addition, the IgE-Fc carbohydrate does not make any contacts across the IgE-Fc diad axis, but lies along the surface of each IgE-Fc domain.

The IgE-Cε3 domains bind at the top of the FcεRIα D1/D2 interface and along the backside of the D2 domain. The receptor contains two distinct binding sites for the two Cε3 domains. Site 1 refers to the interaction of one Cε3 domain exclusively with the C-C′ region of the receptor D2 domain, as indicated, while Site 2 refers to the interaction of the second Cε3 domain with the top of the receptor at the D1/D2 interface (FIG. 1b,c). Site 1 is centered around Y131 on the C′ loop in the receptor D2 domain. Site 2 is located at the top of the receptor and involves four surface-exposed tryptophans (W87, W110, W113, and W156). The two chains of the Fc molecule bind the receptors using surface loops in Cε3 that are distal to the Cε4 domains. These loops are the immunoglobulin-fold BC (362-364), DE (394-395), and FG (424-427) loops, in addition to residues in the Cε2/Cε3-linker region near the interchain disulfide (328-336). The linker regions between the Cε2 and Cε3 domains are involved in interactions with the FcεRIα, which cause both linker segments to point up and away from the complex interface. The role of the IgE-Fc Cε4 domains is to provide a structural dimerization scaffold that enables two Cε3 domains to form the bivalent interaction with FcεRIoα.

D. Structural Basis for the Formation of a 1:1 Complex

Biophysical studies of the IgE-Fc:FcαFRα complex in solution indicate that a 1:1 complex is formed between the antibody and FcεRI²⁰⁻²³. This contrasts with models with a 2:1 stoichiometry that have been proposed for the interaction of the IgG antibody with the FcγRIIa and FcγRIIb receptors²⁴⁻²⁶, as well as with the crystal structure of the MHC-class I like neonatal Fc receptor with IgG²⁷⁻²⁹. The observation of a 1:1 complex in both of the IgE-Fc:FcεRIα complex crystal forms is consistent with data on these complexes obtained using gel filtration and analytical ultracentrifugation techniques^{22,23}. In principle, the 1:1 stoichiometry could arise due to FcεRI-induced conformational changes in the IgE-Fc, creating asymmetry in the Fc region, or by the binding of FcεRI across the Fc two-fold axis, creating a steric inhibition for the binding of a second receptor.

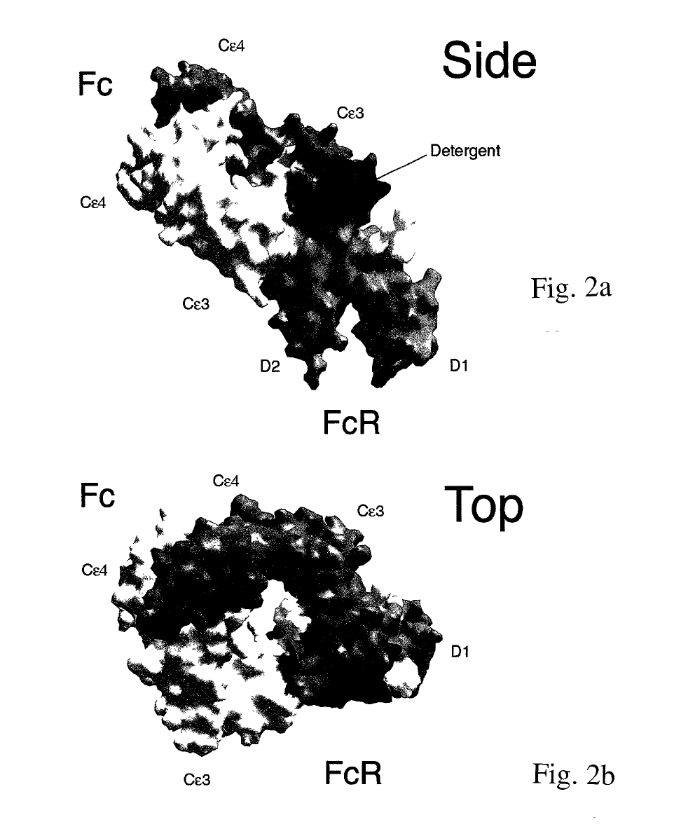

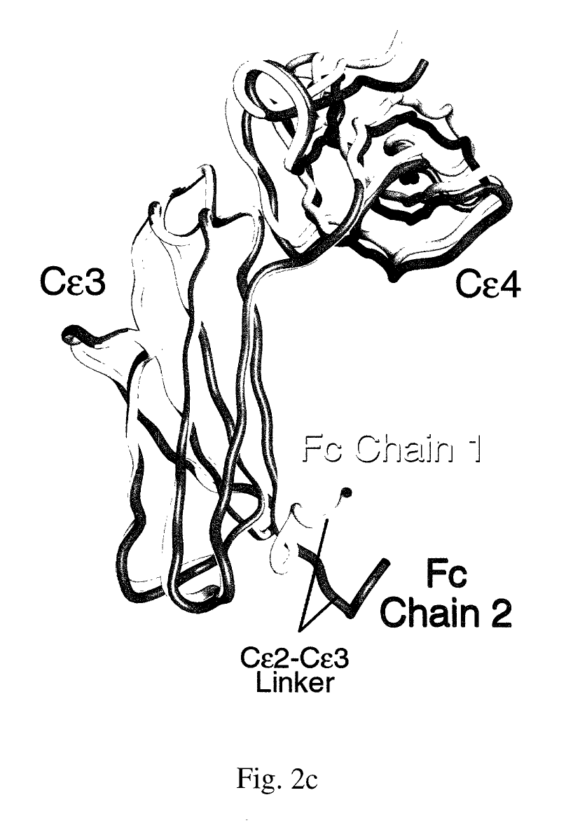

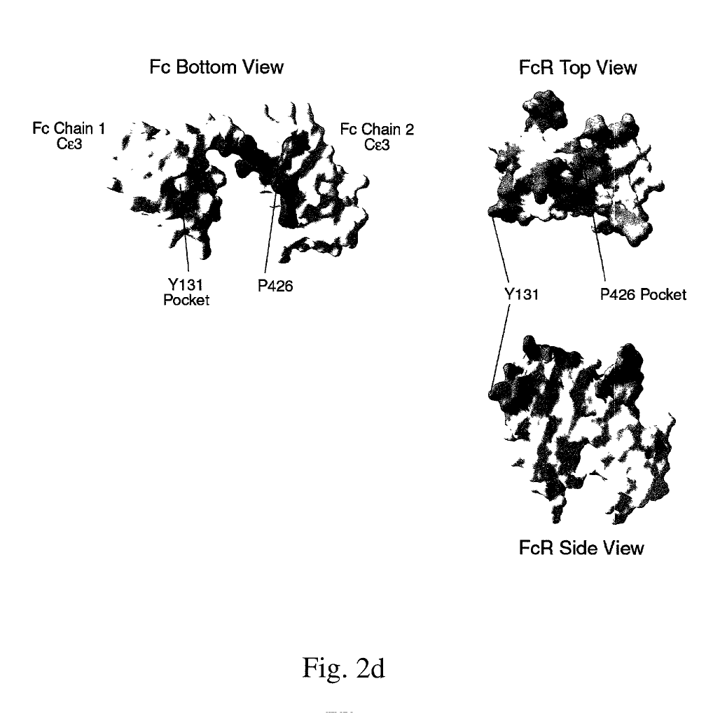

FIGS. 2a and 2b show surface representations of the IgE-Fc:FcεRIα complex, demonstrating how the convex surface of the receptor interacts asymmetrically with the two IgE-Fc Cε3 domains. The receptor is positioned near the Fc-diad axis. There are two structural keys that dictate the formation of complexes with this stoichiometry: (1) The induction of structural asymmetry in the IgE-Fc Cε2/Cε3 linker and (2) Steric hindrance that blocks the binding of a second receptor.

Structural differences in the IgE-Fc domains are easily visualized by the superposition of the two Cε3 domains as shown in FIG. 2c. This superposition demonstrates that the Cε2/Cε3 linker regions comprised of residues 327-336, are constrained to an asymmetric arrangement by interactions with FcεRI. Other loops that are involved in distinct interactions with the two FcεRI binding sites also adopt slightly different conformations in the two Cε3 domains, such as the FG loops indicated in FIG. 2c.

Binding of one receptor to sites 1 and 2 creates a steric block of the binding of a second receptor. FIG. 2d shows representations of the both the IgE-Fc and FcεRIα in which the complex has been separated to exposed the buried interaction surfaces. The Cε2/Cε3 linker amino acids form the top of an arch that conforms to the convex surface of FcεRIα, generating an asymmetric binding site for a single receptor. While some of the Cε3 binding surface remains accessible to the interaction with a second receptor, superposition of a second receptor onto the 1:1 complex shows significant steric overlap between receptors and the IgE-Fc Cε2/Cε3 linker amino acids. Thus the binding of one receptor effectively prevents the binding of a second due to both the asymmetric arrangement of the IgE-Fc Cε2/Cε3 linker and by receptor binding across the Fc diad axis. Both contribute sterically to interfering with the binding of a second receptor. Although different residues in the Fc are used to form sites 1 and 2, there are four residues (R334, G335, V336, and H424) common to both sites, providing direct interactions that prevent the simultaneous binding of two receptors to one IgE-Fc.

E. Structural Changes in the Receptor and IgE Conformations upon Binding.

The receptor shows little change in conformation upon complex formation with the Fc. The overall RMS difference in 158 Cα positions compared to the unbound receptor¹¹ is 1.11 Å. There are two loops on the receptor which adopt different conformations from those seen in the original FcεRIα structure¹¹, the BC loop in D1 (residues 30-35) and the C′ strand in D2 (residues 127-133). The D2 C′ strand is longer in the FcεR1α:IgE-Fc complex compared to the FcεRIα structure alone. In the receptor structure, the C strand forms hydrogen bonds to the C′ strand through residue L127¹¹, while in the complex, the main chain hydrogen bonds extend to Y 131. However, analysis of the FcεRIα structure in multiple crystal forms (Garman et al., in preparation) shows that the C′ strand can adopt a variety of conformations depending on the chemical environment. The BC loop in Domain 1 also adopts different conformations in different crystal forms, but this region is not involved in IgE-Fc interactions.

The IgE-Fc in the complex is observed in a conformation that is very similar to the Fc domains of IgG antibodies^{30,31}. Similar binding interactions between IgG antibodies and FcγRs could form an analogous 1:1 complex, as suggested by biophysical studies of the IgG-Fc interaction with FcγRIII³². In contrast to the similarities of the bound IgE-Fc to IgG-Fc structures, the crystal structure of the IgE-Fc alone shows a large re-arrangement of the two Cε3 domains that is greater than the conformational variation observed in IgG-Fc structures (see P_AL-9, ibid.). The IgE-Fc conformation may change substantially from the unbound conformation, which may exist in multiple conformational states that interact weakly with the receptor. This conformational variation in the IgE-Fc structure suggests new avenues to inhibiting IgE-receptor interactions using allosteric modulators that could stabilize the closed, unbound IgE-Fc structure.

F. Details of the Binding Surfaces of the FcR:IgE Interaction

The surface areas of both the IgE-Fc and FcεRIα that are involved in binding are shown in FIG. 2d, forming a total buried surface of ˜1890 Å². The IgE-Fc adopts a concave or crown-like configuration at the N-terminal ends of the two Cε3 domains that matches the convex shape of the receptor, with the top of the crown defined by the Cε2/Cε3 linker residues. The two Cε3 domains form two distinct sets of interactions with the receptor that involve an overlapping but non-identical set of IgE residues in each of these two sites. Of the fifteen FcεRI residues that contact the IgE-Fc, seven are aromatic and five of these aromatic residues are surface exposed tryptophans. In contrast, of the nineteen IgE-Fc residues that contact the FcεRIα, none are aromatic. The large fraction of aromatic receptor residues that are involved in this interaction and the large buried surface area may both contribute to the stability of the complex (K_{D}˜10⁻⁹-10⁻¹⁰ M).

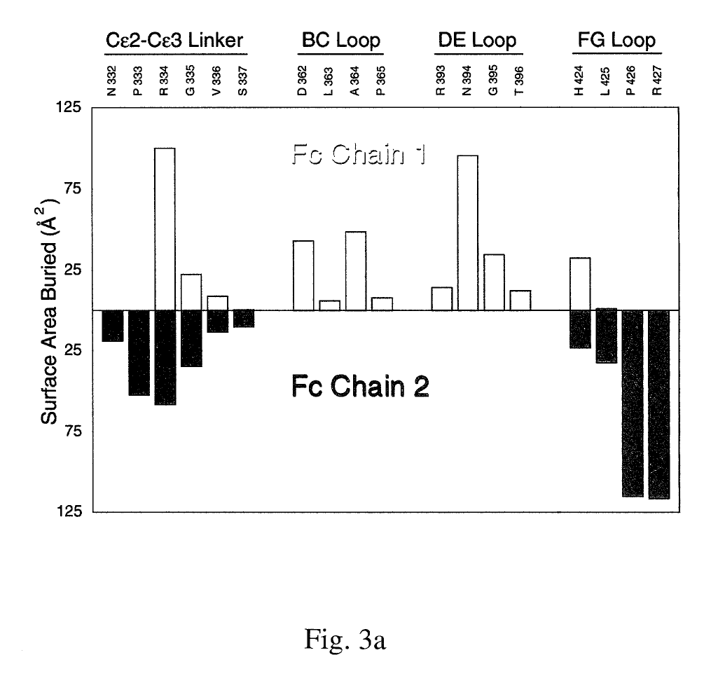

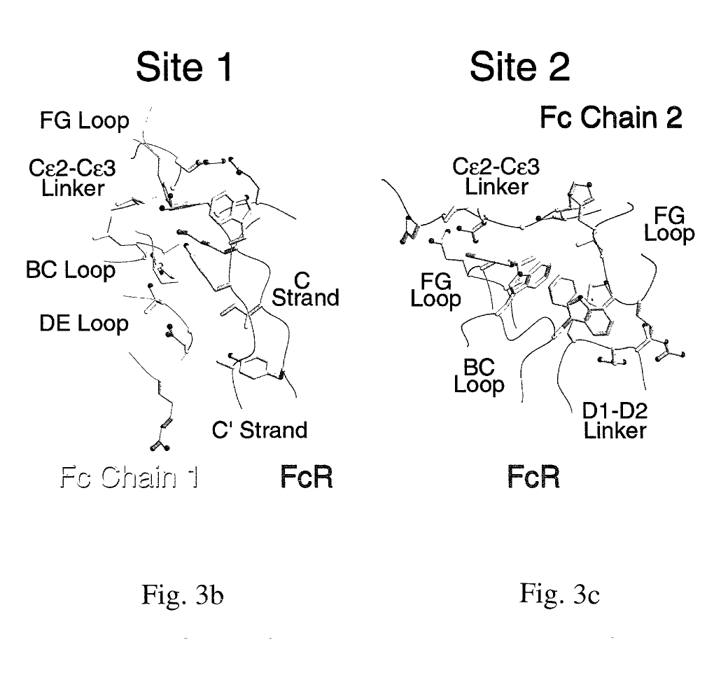

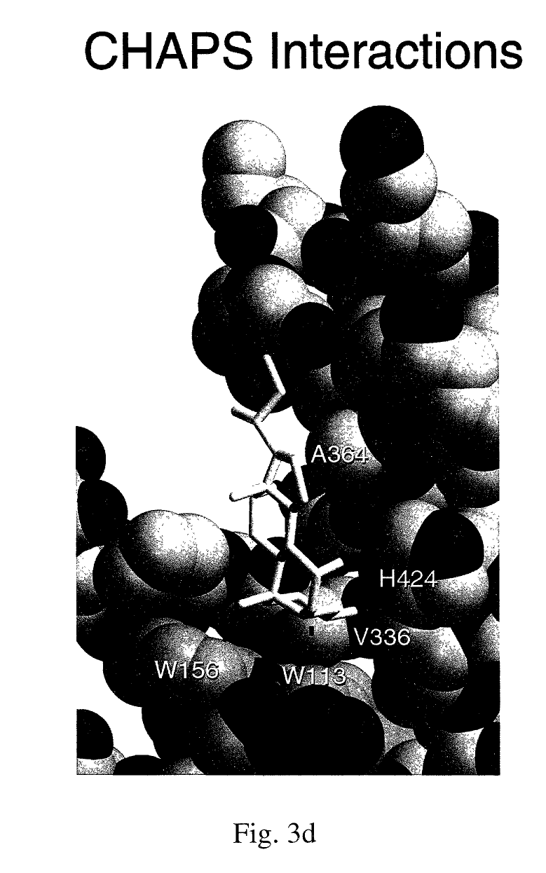

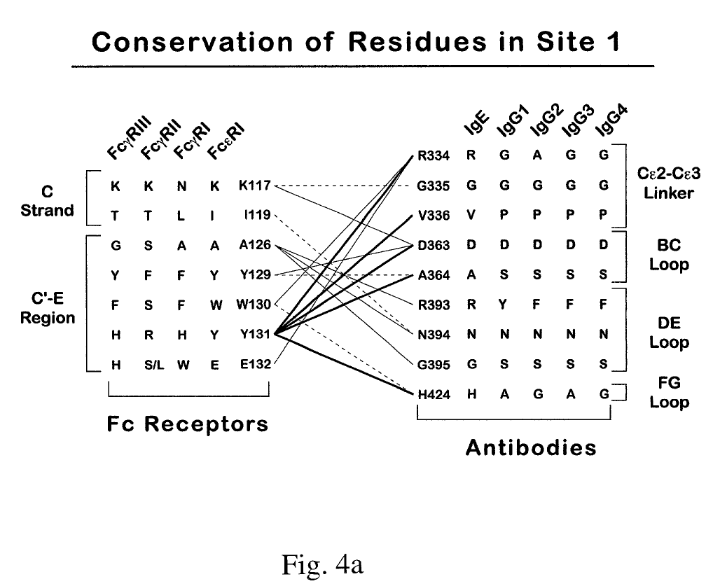

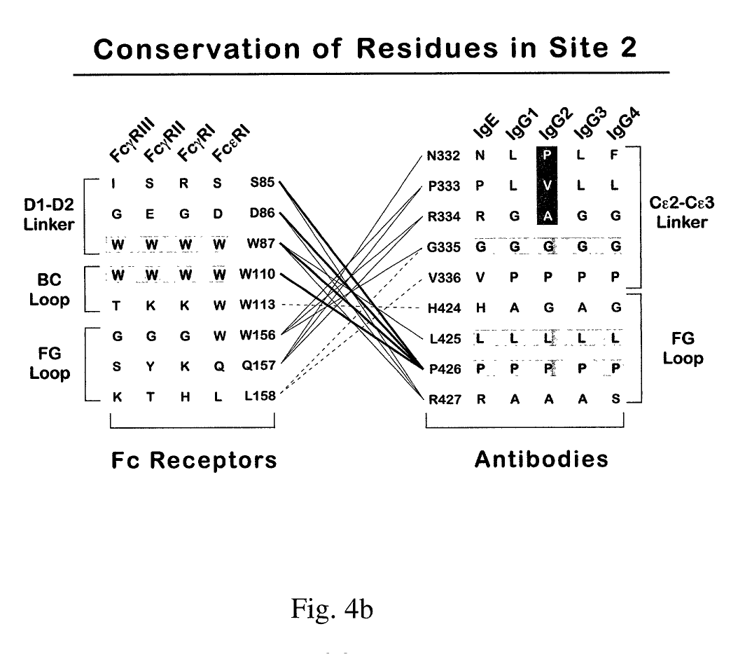

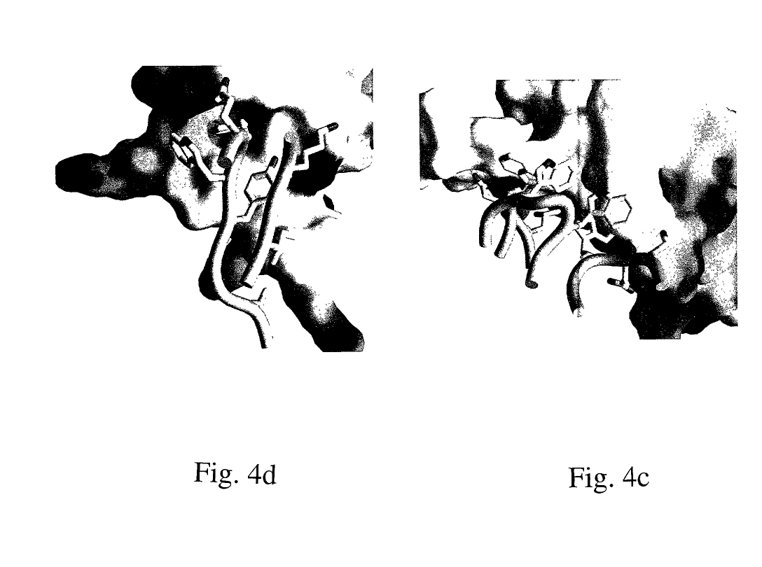

FIG. 3a shows a plot of the IgE-Fc residues that are buried in the interaction with the receptor. Cε3 residues involved in Site 1 are in the top half of the plot and form specific interactions with FcεRIα residues shown in FIG. 3b. Nine amino acids from the IgE and seven amino acids from the receptor form Site 1 (FIG. 3b and FIG. 4a), burying a total of ˜835 Å² of surface area. The IgE residues are from four distinct regions of the IgE-Fc sequence that are predominantly loop and adjacent strand residues, including the N-terminal linker (residues 334-336), the BC loop (residues 362-364), the DE loop (residues 394-395) and the FG loop (residue 424). The receptor residues derive from two regions of the D2 domain, involving the C strand (residues 117 and 119) and the flexible C′-E region (residues 126 and 129-132). Two potential salt bridges (αK1 17-Cε3D362 and αE132-Cε3R334) and 4 potential hydrogen bonds (αK117-Cε3G335, αY129-Cε3D362, αY131-Cε3D364 and (αY131-Cε3H424) are formed across the Site 1 interface (FIG. 3b, FIG. 4a).

The Cε3 residues that are buried in the formation of Site 2 are shown in the bottom panel of FIG. 3a, FIG. 3c and in FIG. 4b. Residues R334, G335, V336, and H424 are buried in both Site 1 and Site 2 interfaces (FIG. 3a) but the remaining residues are unique to each of the two binding sites. Site 2 is larger than Site 1, with 10 amino acids from the IgE and 8 amino acids from the receptor forming a buried interface of 1040 Å². The IgE residues are localized to two distinct regions of the sequence, including extensive interactions with the Cε2/Cε3-linker region (residues 332-336) and the FG loop (residues 424-427). The FcεRIα residues are from three regions of the sequence (FIGS. 3c and 4b), the D1D2 linker region (residues 85-87), the BC loop (residues 110 and 113) and the FG loop (residues 156-158). Residues from the receptor D1 domain do not form direct interactions with the IgE-Fc, but are likely important for stabilizing the conformation of the D1D2 linker residues, including the highly conserved W87 (FIGS. 3c and 4b). In contrast to the Site 1 interface, Site 2 contains primarily hydrophobic amino-acids with limited polar interactions. Site 2 involves 3 potential hydrogen bonds across the interface (αW156-Cε3G335, αQ157-Cε3N332 and αQ157-Cα3R334). The large amount of buried hydrophobic surface area may contribute to the high affinity binding constant.

G. Electron density appears for CHAPS detergent molecules in the Form II crystals.

One of these molecules sits above FcεRI-W156 and below the Cε3-FG loop near H424 in Site 2 (FIG. 3d). The position of the CHAPS heterocyclic core is analogous to the position of the FcR C′ loop residues in Site 1. Although the CHAPS interaction may be weak, this structure provides a foundation for using combinatorial synthetic chemistry methods to improve these initial binding interactions^{33,34}. A high affinity inhibitor of the Site 1 interactions could prove to be a viable inhibitor of the IgE binding, given mutagenesis data that indicate the importance of this site in overall IgE:FcεRI affinity. In addition, H424, which is located next to the CHAPS binding site, makes contacts with the receptor in both Site 1 and Site 2. A small molecule inhibitor that could interact with both the Y131 pocket of Cε3 (site 1) and with H424 might effectively disrupt both Site 1 and Site 2 interactions with the receptor.

H. Locations of IgE and FcR Mutations in the Structure of the Complex.

Mutagenesis studies of both the IgE-Fc and FcεRIα have been carried out in efforts to define the residues in both proteins that contribute to the stability of the complex. For FcεRIα, these studies have implicated residues located in the D2 domain, including amino acids 87, 113, 115, 117, 118, 120, 121, 122, 123, 128, 129, 130, 131, 132, 149, 153, 155, 156, 159, 160, 161^{11,35-39}. While the general location of these residues is consistent with the observed complex, not all of the residues make direct contacts with the IgE-Fc, as shown in FIGS. 4a and 4b. Of the residues identified by mutagenesis techniques, eight are observed to interact directly with the IgE (87, 113, 117, 129, 130, 131, 132, 156), twelve are within three residues that interact (115, 118, 120, 121, 122, 123, 128, 153, 155, 159, 160, 161) and the remaining amino acid (149) is buried and forms part of the hydrophobic core of D2.

The identification of the IgE-Fc binding site for receptor has implicated regions near the Cε2/Cε3 linker, the Cε3-AB helix and the Cε3-CD loop^{12,15,16,40,41}. In general, most studies concur that the Cε2 and Cε4 domains do not interact directly with antibody. Residues in the IgE-Fc AB helix are likely to have an indirect effect on receptor binding, by altering the flexibility and geometry of the Cε3/Cε4 interface. Mutagenesis techniques have identified residues 333, 334, 376, 378, 380, 393, 414, 427 and 430 as possible contact residues in the IgE-Fc. Of these residues, three are observed as contact residues (333, 334, 427), one is within three residues (430) of a contact. However, four of these residues are located in the CD loop of Cε3 and are distant from the IgE-Fc:FcεRIα interface (376, 378, 380, 414). Not all mutations at these residues are deleterious, for example R376A or R376K has little effect on binding, while R376E reduces the binding to receptor. Similarly, D409A, D409E or D409N are well tolerated, while D409R disrupts receptor binding. Thus it is possible that these selective mutations have an indirect effect on receptor binding, potentially through alterations in the conformation of the Cε3 domain.

I. The basis for IgE Specificity and Implications for Other Receptor:Antibody Complexes

FIGS. 4a and 4b show schematic diagrams of the amino acid residues that lie within 4 Å of each other in the Site 1 and Site 2 interfaces. Direct contacts are indicated by the connecting lines, which highlight residues that form the largest number of atomic contacts across the respective interfaces. Also shown are the residues that are found in the related human IgG receptors (FcγR1, FcγRII and FcγRIII, to the left) and in four subtypes of IgG antibodies (to the right).

In Site 1 there is little conservation of the residues that form the IgE-Fc:FcεRIα interface. Three residues are completely conserved (IgE residues 335, 362 and 394) in the Fc sequences, while there is poor conservation in the receptor sequences, except for the partial conservation of K117 and the relatively conserved Y129 (either Y or F). Interestingly, the conservation of K117 in three of the four receptors matches the complete conservation of D362 and G335, potentially preserving one of the two Site 1 salt bridges and one of the Site 1 hydrogen bonds. The conservative substitution of Y129 for F or Y in the IgG receptors also suggests that this site may be found in IgG-Fc complexes with the FcγRs. However, Y131, which forms a large number of atomic contacts across the interface and is buried in a shallow surface pocket on the IgE-Fc, is not conserved in the FcγRs (changing to either H or R). Given the central location of Y131 to the IgE interface, this residue may play an important role in immunoglobulin class specificity (FIG. 3b). For example, four of the five contact residues in IgE for Y131 are also different in the IgG-Fc sequences. In general, residues within the four IgG subtypes are highly conserved in the Site 1 interface (7/9 identical), as compared to the significant variation in the FcγR residues. FIG. 4b shows the conservation of interactions that are central to the Site 2 interface. P426 and L425 are absolutely conserved in all IgG Fc sequences and P426 interacts with two absolutely conserved tryptophans in the FcεR complex (W87 and W110). The two tryptophans form a hydrophobic pocket on the surface of the receptor into which the proline inserts (FIGS. 3c and 4c). Site 2 also includes three residues (IgE residues 332-334) that have been shown to affect binding of IgG subtypes to FcγRI. IgG1 binds with high affinity to FcγRI, whereas IgG2 does not, and the difference in binding affinity can be introduced into IgG1 by the substitution of residues LLG to PVA (IgE residues 332-334, highlighted in black in FIG. 4b)⁴²,⁴³. This region of the IgE-Fc interacts with the FcεRIα FG loop residues 156-158 (FIGS. 3c and 4b). Previous mutagenesis experiments have also shown that the transfer of the FcεRIα FG loop to FcγRII confers detectable IgE binding⁴⁴. Thus, residues involved in the formation of Site 2 are implicated in the binding and specificity of both IgE and IgG FcRs, consistent with a conserved binding mode across these members of the FcR family. Overall, five residues are completely conserved in these human receptors and IgG sequences that could form a common set of contacts. Variation in the FcγR FG loop sequences that contact the N-terminal linker region of the Fc fragment may provide key interactions that modulate the affinity of interaction of specific FcR:IgG pairs.

J. Conclusions

The crystal structure of the IgE-Fc:FcεRIα complex clarifies the atomic interactions that regulate the specificity and stoichiometry of protein:protein interactions underlying allergy and anaphylaxis. Similar complexes may form between IgG antibodes with their receptors, as suggested by previous mutagenesis studies and the structural analysis presented here, in contrast to models proposed for the interaction of IgG-Fc with the low affinity receptor, FcγRIIb²⁵ and FcγRIIa²⁴. Knowledge of these interactions may allow the development of inhibitors for the treatments of allergy and asthma and may also facilitate the targeted engineering of therapeutic antibodies to interact with specific subsets of the FcR family⁴⁵.

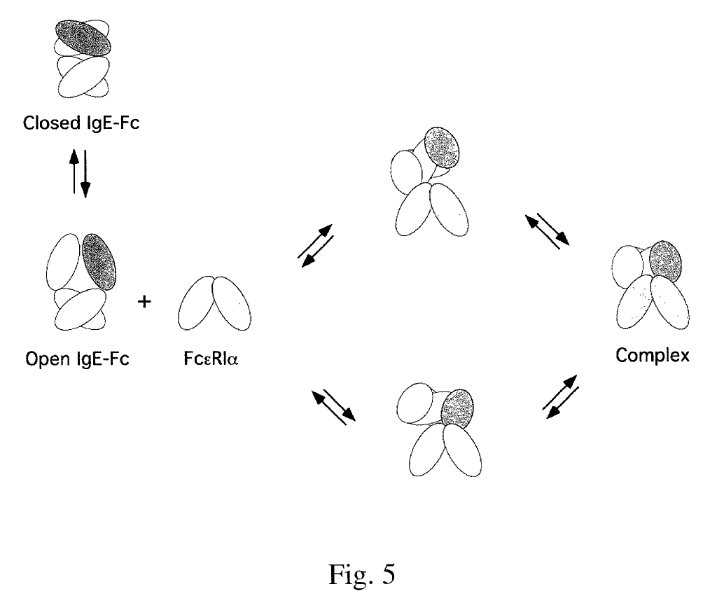

The observed flexibility in the IgE Cε3/Cε4 hinge (see 60/189,403, ibid.) and the distinct interactions of the two Cε3 domains in Site 1 and Site 2, are consistent with a kinetic scheme for IgE binding shown in FIG. 5. In this scheme, the independent binding of each Cε3 domain in the FcεRIα complex, leads to two pathways for the full dissociation of the complex. Surface plasmon resonance studies of IgE-Fc dissociation show two distinct kinetic dissociation rates that were hypothesized to represent the interaction of two different binding interactions between the IgE-Fc and FcεRIα, consistent with this kinetic scheme^{16,41}. The IgE-Fc mutation R334S affects the biphasic dissociation kinetics of the IgE-Fc:FcεRIα complex by selectively accelerating the slow dissociation rate¹⁶. R334 is used in distinct and specific ways in Site 1 and Site 2, forming a salt bridge in Site 1 and van der Waals contacts in Site 2, consistent with the observation that one of these interactions could be more sensitive to the R334S mutation. The two dissociation pathways shown in FIG. 5 could exhibit two distinct overall kinetic rates that could be selectively affected by the R334S mutation. If the two Cε3 domains bind independently, with transient exposure of each site in the complex, inhibitors for either Site 1 or Site 2 could potentially accelerate the dissociation of receptor-bound IgE. Such inhibitors might prove useful in the treatment of acute allergic reactions in which dissociation of mast-cell associated IgE would be beneficial.

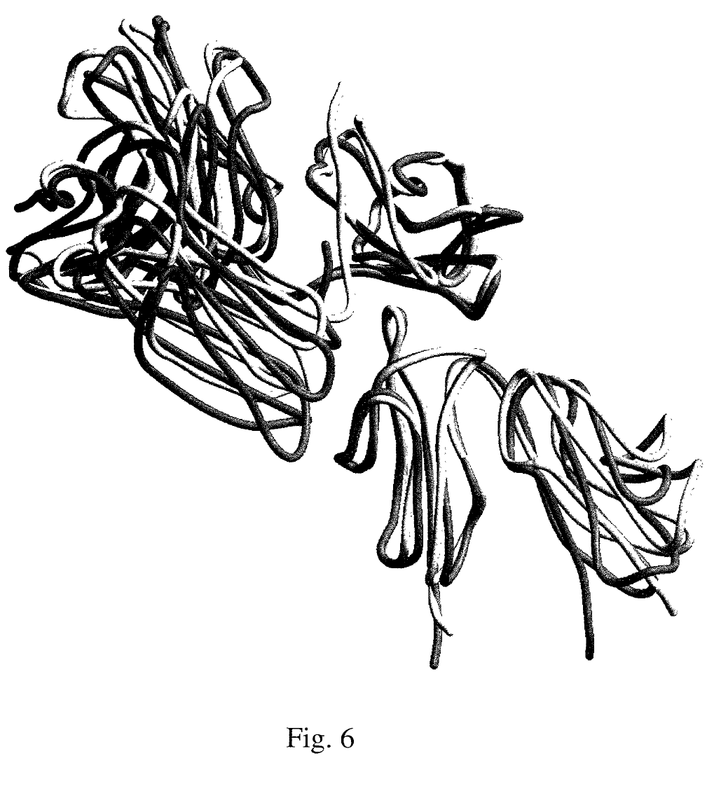

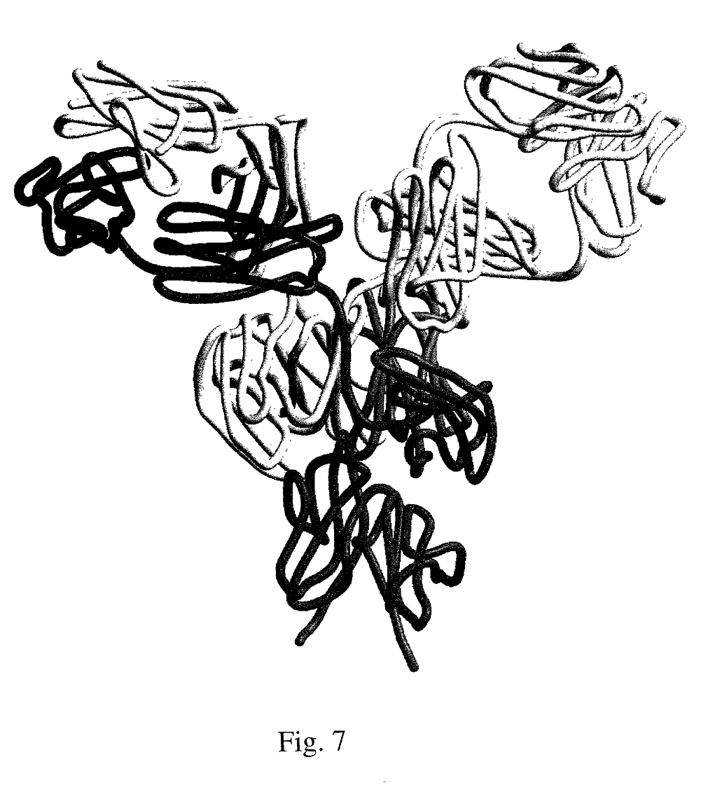

A model for the formation of a complex between an intact IgG antibody and Fc-receptor is shown in FIG. 6. In this model the crystal structure of the low affinity IgG receptor (FcγRIIb)²⁵ and one of the available intact IgG antibody structures (1IGY)⁴⁶ were superimposed on the IgE-Fc:FcεRIα complex. Superposition of the IgG structure is based on the Site 2 interactions, and this places the second IgG-Fc Cg2 domain within close proximity of the Site 1 binding surface without any conformational rearrangements (FIG. 6). The Fab arms of IgG are flexible and are also easily accommodated into this complex. Antigen-induced crosslinking of antibody:FcR complexes, leads to the co-localization of Fc receptors and the initiation of intracellular signal transduction cascades^{2,47}. Within the one of the IgE-Fc:FcεRIα crystal forms and the IgE-Fc crystals (60/189,403, ibid.), Cε3 domains from adjacent molecules are observed to form packing interactions in the crystal through a strand to strand hydrogen-bonding interaction. Such interactions could potentially play a role in orienting crosslinked receptors, allowing the intracellular approach of receptor-associated kinases to adjacent γ-chain cytoplasmic tails, initiating the signal transduction cascade. A potential role for Cε3:Cε3 interactions in signal transduction remains to be tested.

K. Methods

1. Crystallization of the Human IgE-Fc:FcεRIα Complex

Human IgE-Fc Cε3/Cε4 domains and a carbohydrate mutant of the FcεRIα¹¹ were expressed in insect cells essentially as described for IgE-Fc Cε3/Cε4 in 60/189,403. Complexes of wt-Fc-Cε3/Cε4 and wt-FcεRIα produced only poorly diffracting crystals. Since the receptor is heavily glycosylated (˜33% carbohydrate by weight), and the carbohydrate sites are dispersed on the receptor surface, a subset of these attachment sites was removed to improve the protein crystallization. A previously-described carbohydrate mutant of the receptor¹⁷ lacking three of the seven wild type carbohydrate sites (residues 74, 135, and 140) located on both D1 and D2 in the receptor structure. The triple receptor mutant, FcεRIαΔ4-6 was subcloned into the pvl1392 baculovirus transfer vector and recombinant virus produced. The mutant receptor was active, expressed well and was purified by affinity chromatography similarly to the wt protein. Purified wt-Fc and (αΔ4-6 or wt-α were incubated to form complex, which was subsequently purified by gel filtration chromatography using a Pharmacia Superdex 75 column and concentrated to 10 mgs/ml. Crystallizations were carried out using the the hanging drop method of vapor diffusion. Crystals of the wtIgE-Fc:wt-FcεRIα complex were grown from 1.4-1.6M Ammonium Sulfate, 100 mM Tris pH 8.5, over a period of 8-12 months (Form I). Purified wtIgE-Fc:αA4-6 complex was crystallized using 100 mM Tris, pH 8.5, 1.4-1.6M Ammonium Sulfate, and 8 mM CHAPS at room temperature. Crystals were then moved into harvest buffer (Form I: 2.1-2.7M Ammonium Sulfate, 100 mM Tris pH 8.5 or Form II: 1.6-2.0 M Ammonium Sulfate, 100 mM Tris pH 8.5, and 0.8 mM CHAPS). Crystals were frozen in harvest buffer supplemented with 15% glycerol. Data sets were collected at ALS 5.0.2 beamline and the APS DNDCAT 5-ID-B beamline at −160 C using an ADSC Quantum 4 detector or a MarCCD detector. Images were processed using the DENZO/SCALEPACK programs⁴⁸. Form I crystals belong to spacegroup P4₁2₁2 with cell dimensions a=b=126 Å, c=129 Å and Form II crystals belong to the space group R32 with cell dimensions a=192.8 Å and c=302.4 Å (hexagonal setting). Intensities were adjusted using the TRUNCATE program prior to molecular replacement using the AMoRe⁴⁹ and EPMR programs⁵⁰. 2. Crystal Structure Determination and Refinement

Molecular replacement for the Form II crystal was performed using coordinates from the 2.4 Å structure of the receptor¹¹. The use of normalized structure factors in AMoRe was critical to the success of the search. Both AMoRe and EPMR produced crystallographically equivalent locations for the receptor. 2Fo-Fc electron density maps with phases from the receptor revealed density corresponding to the two Cε3 portions of the Fc. A model for the core residues of Cε3 was created (see 60/189,403, ibid.) based upon homologous residues from an intact IgG structure 1IGT 46 A new 2Fo-Fc map was created with phases from the receptor and core residues of Cε3. This map showed density for the locations of the two Cε4 domains. A model for the core residues in Cε4 was made based upon the homologous residues in 1IGT. Rigid body refinement of the receptor, the core residues in Cε3, and the core residues in Cε4 reduced the Rfree to 45%. 2Fo-Fc maps and composite omit maps revealed clear density for protein and carbohydrate atoms absent from the model. The Form I crystal structure was solved by molecular replacement using the complex model from Form II, with a clear top solution. Given the limited resolution of Form I, refinement was limited to rigid body minimization.

Refinement was continued with the 3.25A Form II data using the CNS program¹⁹. Non-crystallographic symmetry restraints of 300 kcal/mol/A² were imposed on all atoms in the Fc except the loops that interact with the receptor. Refinement was performed using all data from 40-3.25 Å with |F|>0 and using a bulk solvent correction. After inserting all the missing loops from the protein chains, CHAPS molecules were located as large peaks of positive density in Fo-Fc maps. The current refinement statistics are summarized in

TABLE 3
Figures were made using the programs Molscript⁵¹ and Grasp⁵².
Data Collection and Refinement Statistics
Data

| Data Set | Form I | Form II-Low Res. | Form II-High Res. |
|---|---|---|---|
| Resolution (Å)‡ | 30-4.5 (4.66-4.5) | 30.0-4.00 (4.14-4.00) | 40.0-3.25 (3.37-3.25) |
| Source | APS DND 5ID | ALS 5.02 | APS DND 5ID |
| Wavelength (Å) | 1.0000 | 1.2000 | 1.0340 |
| Completeness‡ | 99.5 (97.7) | 99.7 (98.3) | 99.7 (98.7) |
| Ave. Redundancy‡ | 7.0 (5.8) | 5.0 (3.9) | 3.7 (3.4) |
| Rmerge‡ | 17.8 (57.5) | 15.2 (75.0) | 12.4 (90.1) |
| I/sigI‡ | 5.9 (2.0) | 4.4 (2.0) | 13.3 (1.5) |
| observations (unique)‡ | 39925 (5703) | 91617 (18459) | 125663 (34235) |
| # refl in refinement (free) | | 18455 (945) | 34156 (1736) |

Refinement (Form II, 3.25 Å)

| Rfactor/Rfree | Total # atoms | Protein | Carbohydrate | Detergent | Sulfate |
|---|---|---|---|---|---|
| 25.8/28.1 | 5251 | 4821 | 259 | 146 | 25 |
| RMSD | | | Average B | | |
| Bonds | Angles | Overall | Receptor | Fc chain 1 | Fc chain 2 |
| 0.0102 | 1.58 | 91.0 | 63.2 | 94.9 | 99.4 |

Ramachandran

| Favored | Allowed | Generous | Disallowed |
|---|---|---|---|
| 77.0% | 21.5% | 1.5% | 0.0% |

‡Last shell is shown in parentheses
R_{merge}= Σ|Iᵢ-<I>|/Σ|I|, where Iᵢ is the intensity of and individual reflection and <I> is the average intensity of that reflection.
R_{cryst}= Σ|Fₚ|-|F_{c}|/Σ|Fₚ|, where F_{c} is the calculated and Fₚ is the observed structure factor amplitude.

### REFERENCES

1. Metzger, Immunol Rev 125, 37-48 (1992).

2. Kinet, Annu Rev Immunol 17, 931-972 (1999).

3. Daeron, Annu Rev Immunol 15, 203-234 (1997).

4. Turner et al., Nature 402, B24-30 (1999).

5. Ravetch et al., Annu Rev Immunol 16, 421-432 (1998).

6. Holt et al., Nature 402, B 12-17 (1999).

7. Cookson, Nature 02, B5-11 (1999).

8. Moffatt et al. Curr Opin Immunol 11, 606-609 (1999).

9. Jardieu et al., Int Arch Allergy Immunol 118, 112-115 (1999).

10. Chang, Nat Biotechnol 18, 157-162(2000).

11. Garman et al., Cell 95, 951-961 (1998).

12. Nissim et al., Embo J 10, 101-107 (1991).

13. Keegan et al., Mol Immunol 28, 1149-1154 (1991).

14. Presta et al., J Immunol 151, 2623-2632 (1993).

15. Basu et al., J Biol Chem 268, 13118-13127 (1993).

16. Henry et al., Biochemistry 36, 15568-15578 (1997).

17. Letourneur et al., J Biol Chem 270, 8249-8256 (1995).

18. Jones et al., Acta Crystallogr A 47, 110-119 (1991).

19. Brunger et al., Acta Crystallogr D Biol Crystallogr 54, 905-921 (1998).

20. Kanellopoulos et al., J Biol Chem 255, 9060-9066 (1980).

21. Robertson et al., J Biol Chem 268, 12736-12743 (1993).

22. Keown et al., Proc Natl Acad Sci U S A 92, 1841-1845 (1995).

23. Keown et al., Eur Biophys J 25, 471-476 (1997).

24. Maxwell et al., Nat Struct Biol 6, 437-442 (1999).

25. Sondermann et al., Embo J 18, 1095-1103 (1999).

26. Sondermann et al., Biochemistry 38, 8469-8477 (1999).

27. Burmeister et al., Nature 372, 379-383 (1994).

28. Martin et al., Biochemistry 38, 12639-12647 (1999).

29. Sanchez et al., Biochemistry 38, 9471-9476 (1999).

30. Deisenhofer et al., Hoppe Seylers ZPhysiol Chem 357, 1421-1434 (1976).

31. Harris et al., Adv Immunol 72, 191-208 (1999).

32. Ghirlando et al., Biochemistry 34, 13320-13327 (1995).

33. Klekota et al., Trends Biotechnol 17, 205-209 (1999).

34. Freidinger et al., Curr Opin Chem Biol 3, 395-406 (1999).

35. Mallamaci et al., J Biol Chem 268, 22076-22083 (1993).

36. Hulett et al., J Biol Chem 269, 15287-15293 (1994).

37. Hulett et al., J Biol Chem 270, 21188-21194 (1995).

38. Cook et al., Biochemistry 36, 15579-15588 (1997).

39. Hulett et al., J Biol Chem 274, 13345-13352 (1999).

40. Presta et al., J Biol Chem 269, 26368-26373 (1994).

41. Sayers et al., Biochemistry 37, 16152-16164 (1998).

42. Duncan et al., Nature 332, 563-564 (1988).

43. Chappel et al., Proc Natl Acad Sci U S A 88, 9036-9040 (1991).

44. Hulett, et al., Eur J Immunol 23, 640-645 (1993).

45. Isaacs et al., J Immunol 161, 3862-3869 (1998).

46. Harris et al., Nature 360, 369-372 (1992).

47. Vonakis et al., J Biol Chem 272, 24072-24080 (1997).

48. Otwinowski et al., Methods in Enzymology: Macromolecular Crystallography, part A (ed. C. W. Carter, J. R. M. S., Eds) 307-326 (Academic Press, 1997).

49. Collaborative Computational Project, Acta Cryst. D50, 760-763 (1994).

50. Kissinger et al., Acta Crystallogr D Biol Crystallogr 55, 484-491 (1999).

51. Kraulis et al., Journal of Applied Crystallography 24, 946-950 (1991).

52. Nicholls et al., Proteins 11, 281-296 (1991).

While the various embodiments of the present invention have been described in detail, it is apparent that modifications and adaptations of those embodiments will occur to those skilled in the art. It is to be expressly understood, however, that such modifications are adaptations are within the scope of the present invention, as set forth in the following claims.

What is claimed is:

1. A three-dimensional model selected from the group consisting of: (a) a three-dimensional model of a complex between (i) an extracellular domain of a human high affinity Fc epsilon receptor alpha chain (FcεRIα) protein and (ii) a human IgE Fc region comprising Cε3 and Cε4 domains (Fc-Cε3/Cε4), wherein said model substantially represents the atomic coordinates specified in Table 1; and (b) a three-dimensional model comprising a modification of said model of (a), wherein said modification represents a complex between a Fc receptor protein that binds to a Fc domain of an antibody and an antibody Fc region that binds to a Fc receptor protein the hinge between domain Cε3 and domain Cε4 of the Fc-Cε3/Cε4 region, and a FcεRIα:Fc-Cε3/Cε4 region that interacts with 3-[3-(cholamidopropyl) dimethylammonio]-1-propane-sulfonate (CHAPS).

2. A method to produce the three-dimensional model of claim 1, wherein the three-dimensional model is the complex between the extracellular domain of a human FcεRIα protein and a human Fc-Cε3/Cε4 region, said method comprising representing amino acids of said protein and said region in said complex at substantially the atomic coordinates specified in Table 1.

3. A method to produce the three-dimensional model of a complex between (i) an extracellular antibody binding domain of an antibody receptor protein other than human FcεRIα as represented by coordinates in Table 1 and (ii) an antibody receptor binding domain of an antibody other than human IgE as represented by coordinates in Table 1, said method comprising homology modeling.

4. An isolated crystal of a complex between an extracellular domain of a FcεRIα protein and an IgE Fc-Cε3/Cε4 region.

5. A method to produce the isolated crystal of claim 4, wherein the complex is between an extracellular domain of a FcεRIα protein and an IgE Fc-Cε3/Cε4 region, said method comprising vapor diffusion.

6. A method to identify a compound that inhibits the binding between an IgE antibody and a FcεRIα protein, said method comprising using a three-dimensional model of a complex between an extracellular domain of a human high affinity FcεRIα protein and a human Fc-Cε3/Cε4 region to identify said compound, wherein said model substantially represents the atomic coordinates specified in Table 1.

7. An inhibitory compound identified in accordance with the method of claim 6.

8. A therapeutic composition comprising an inhibitory compound of claim 7.

9. A method to protect an animal from allergy, said method comprising administering to said animal an inhibitory compound of claim 7.

10. A compound that inhibits the binding between an IgE antibody and a FcεRIα protein, said compound identified by analysis of a three-dimensional model of a complex between an extracellular domain of a human high affinity FcεRIα protein and a human Fc-Cε3/Cε4 region to identify said compound, wherein said model substantially represents the atomic coordinates specified in Table 1.

11. A polypeptide selected from the group consisting of a FcεRIα:Fc-Cε3/Cε4 interaction site 1, a FcεRIα:Fc-Cε3/Cε4 interaction site 2, a C strand of domain 2 of FcεRIα, a C′E loop of domain 2 of FcεRIα, a tryptophan-containing hydrophobic ridge of FcεRIα, a crystal contact cluster involved in IgE binding; a FG loop in D2; a D1D2 interface; a cleft between D1 and D2; a domain 1; a domain 2; a hydrophobic core; a A′B loop of D1; a EF loop of D1; a BC loop of D2; a CC′ loop of D2; and a strand of D2.

12. An isolated nucleic acid molecule encoding the polypeptide of claim 11.

13. A method of using the three-dimensional model of claim 1, comprising:
(a) analyzing the three-dimensional model substantially representing the atomic coordinates specified in Table 1 to identify at least one amino acid of a target protein represented by said three-dimensional model which if replaced by said identified amino acid(s) to improve a function of said target protein; and
(b) replacing said identified amino acid(s) to produce a mutein having at least one of said improved function.

14. The method of claim 13, wherein said target protein is a Fc-Cε3/Cε4 protein, a FcεRIα protein or a protein comprising SEQ.ID NO.2, and wherein said improved function is selected from the group comprising: (a) increased stability, increased affinity for an IgE binding domain of a FcεRIα protein, altered substrate specificity or increased solubility when said target protein is the Fc-Cε3/Cε4 protein; and (b) increased stability, increased affinity for an Fc-domain of an antibody, altered substrate specificity or increased stability when said target protein is the FcεRIα protein or the protein comprising SEQ.ID NO.2.

15. A mutein produced by the method of claim 14, wherein said mutein has at least one improved function compared to the target protein, wherein said target protein is the FcεRIα protein, the Fc-Cε3/Cε4 protein or the protein comprising SEQ.ID NO.2.

16. The mutein of claim 15 having an improved function compared to an unmodified FcεRIα protein, wherein the amino acid sequence of said mutein differs in at least one position from the amino acid sequence of said unmodified protein, said position being in a region selected from the group consisting of a crystal contact cluster, a tryptophan-containing hydrophobic ridge, a FG loop in D2, a D1 D2 interface, a cleft between D1 and D2, a domain 1, a domain 2, a hydrophobic core, a A′B loop of D1, a EF loop of D1, a BC loop of D2, a C strand of D2, a CC′ loop of D2, a C′E loop of D2, a strand of D2, the amino terminal five residues of said protein, and the carboxyl terminal five residues of said protein.

17. An isolated nucleic acid sequence encoding a mutein of claim 15.

18. A recombinant virus comprising said nucleic acid sequence of claim 17.

19. A recombinant cell comprising said nucleic acid sequence of claim 17, wherein said cell is capable of expressing said nucleic acid sequence.

20. A diagnostic reagent comprising a mutein of claim 15.

21. A therapeutic composition comprising a mutein of claim 15.

22. A method to use a mutein of claim 15, wherein said method is selected from the group consisting of: (a) a method to protect an animal from allergy, said method comprising administering a therapeutic composition comprising said mutein to said animal; (b) a method to detect allergy, or susceptibility thereto, in an animal, said method comprising using said mutein to detect said allergy; and (c) a method to enhance the performance of an IgE binding assay, said method comprising incorporating into said assay said mutein.


SEQUENCE LISTING (6 sequences)

SEQ ID NO: 1 - DNA, 528 bp, Homo sapiens
  CDS (1)..(528)
gtccctcagaaacctaaggtctccttgaaccctccatggaatagaatatttaaaggagagaatgtgactcttacatgtaatgggaacaatttctttgaagtcagttccaccaaatggttccacaatggcagcctttcagaagagacaaattcaagtttgaatattgtgaatgccaaatttgaagacagtggagaatacaaatgtcagcaccaacaagttaatgagagtgaacctgtgtacctggaagtcttcagtgactggctgctccttcaggcctctgctgaggtggtgatggagggccagcccctcttcctcaggtgccatggttggaggaactgggatgtgtacaaggtgatctattataaggatggtgaagctctcaagtactggtatgagaaccacaacatctccattacaaatgccacagttgaagacagtggaacctactactgtacgggcaaagtgtggcagctggactatgagtctgagcccctcaacattactgtaataaaagctccgcgtgagaag

SEQ ID NO: 2 - protein, 176 aa, Homo sapiens
VPQKPKVSLNPPWNRIFKGENVTLTCNGNNFFEVSSTKWFHNGSLSEETNSSLNIVNAKFEDSGEYKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKAPREK

SEQ ID NO: 3 - DNA, 528 bp, Homo sapiens
  CDS (1)..(528)
gtccctcagaaacctaaggtctccttgaaccctccatggaatagaatatttaaaggagagaatgtgactcttacatgtaatgggaacaatttctttgaagtcagttccaccaaatggttccacaatggcagcctttcagaagagacaaattcaagtttgaatattgtgaatgccaaatttgaagacagtggagaatacaaatgtcagcaccaacaagttgctgagagtgaacctgtgtacctggaagtcttcagtgactggctgctccttcaggcctctgctgaggtggtgatggagggccagcccctcttcctcaggtgccatggttggaggaactgggatgtgtacaaggtgatctattataaggatggtgaagctctcaagtattggtatgagaaccacgctatctccattacaaatgccgcagctgaagacagtggaacctactactgtacgggcaaagtgtggcagctggactatgagtctgagcccctcaacattactgtaataaaagctccgcgtgagaag

SEQ ID NO: 4 - protein, 176 aa, Homo sapiens
VPQKPKVSLNPPWNRIFKGENVTLTCNGNNFFEVSSTKWFHNGSLSEETNSSLNIVNAKFEDSGEYKCQHQQVAESEPVYLEVFSDWLLLQASAEVVMEGQPLFLRCHGWRNWDVYKVIYYKDGEALKYWYENHAISITNAAAEDSGTYYCTGKVWQLDYESEPLNITVIKAPREK

SEQ ID NO: 5 - DNA, 669 bp, Homo sapiens
  CDS (1)..(666)
gcggatccctgtgattccaacccgagaggggtgagcgcctacctaagccggcccagcccgttcgacctgttcatccgcaagtcgcccacgatcacctgtctggtggtggacctggcacccagcaaggggaccgtgaacctgacctggtcccgggccagtgggaagcctgtgaaccactccaccagaaaggaggagaagcagcgcaatggcacgttaaccgtcacgtccaccctgccggtgggcacccgagactggatcgagggggagacctaccagtgcagggtgacccacccccacctgcccagggccctcatgcggtccacgaccaagaccagcggcccgcgtgctgccccggaagtctatgcgtttgcgacgccggagtggccggggagccgggacaagcgcaccctcgcctgcctgatccagaacttcatgcctgaggacatctcggtgcagtggctgcacaacgaggtgcagctcccggacgcccggcacagcacgacgcagccccgcaagaccaagggctccggcttcttcgtcttcagccgcctggaggtgaccagggccgaatgggagcagaaagatgagttcatctgccgtgcagtccatgaggcagcgagcccctcacagaccgtccagcgagcggtgtctgtaaatcccggtaaatga

SEQ ID NO: 6 - protein, 222 aa, Homo sapiens
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK